(19) United States
(12) Patent Application Publication
(10) Pub. No.: US 2019/0382845 A1
(43) Pub. Date: Dec. 19, 2019

(54) PROSTATE CANCER AGGRESSIVENESS BIOMARKERS

(71) Applicant: University of Southern California, Los Angeles, CA (US)

(72) Inventors: Inderbir Singh Gill, Los Angeles, CA (US); Peter Jones, Los Angeles, CA (US); Gangning Liang, Los Angeles, CA (US); Kamilla Mundbjerg, Los Angeles, CA (US)

(21) Appl. No.: 15/999,311

(22) PCT Filed: Feb. 17, 2017
(86) PCT No.: PCT/US17/18517

§ 371 Date: Aug. 17, 2018

Related U.S. Application Data
(60) Provisional application No. 62/296,707, filed on Feb. 18, 2016.

(51) Int. Cl. C12Q 1/6886

(52) U.S. Cl. CPC C12Q 1/6886; C12Q 2600/154; C12Q 2600/112

(57) ABSTRACT

A method of identifying subjects having prostate cancer is provided. The method includes obtaining a sample from a subject, isolating DNA from the sample, determining the methylation status of the DNA, and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The comparison indicates whether the sample is normal, a non-aggressive prostate cancer, or an aggressive prostate cancer.

### CROSS-REFERENCE TO RELATED APPLICATIONS

This application is a 35 USC § 371 National Stage application of International Application No. PCT/US2017/018517 filed Feb. 17, 2017, now pending; which claims the benefit under 35 USC § 119(e) to U.S. Application Ser. No. 62/296,707 filed Feb. 18, 2016, now expired. The disclosure of each of the prior applications is considered part of and is incorporated by reference in the disclosure of this application.

### INCORPORATION OF SEQUENCE LISTING

The material in the accompanying sequence listing is hereby incorporated by reference into this application. The accompanying sequence listing text file, named USC1300-1_ST25.txt, was created on Jun. 6, 2019, and is 551 KB. The file can be accessed using Microsoft Word on a computer that uses Windows OS.

### BACKGROUND OF THE INVENTION

### Field of the Invention

The present invention relates to methods of screening and diagnosing prostate cancer.

### Background Information

Prostate cancer (PC) is the most frequently diagnosed cancer and the second most common cause of cancer deaths in men in the United States. Although PC incidence rates have increased over the past 25 years, mortality rates have largely remained unchanged (www.cancer.gov). The advent of a PC diagnostic test, which measures the level of prostate specific antigen (PSA), has resulted in the discovery of earlier, smaller lesions, as well as increased the need for prognostic tools to avoid overtreatment of indolent PC¹. Localized PC is curable through radical prostatectomy or radiation therapy, albeit often followed by significant side effects, including urinary incontinence (5-20%), erectile dysfunction (30-70%), and bowel toxicity (5-10%)^{2,3}. Generally, PC is a slow-growing malignancy with decades of indolence, but takes on an aggressive form displaying rapid growth, dissemination, and lethality in a subset of cases (<20%)^{1,4}.

Predicting tumor progression is critical for clinical decision-making. PC is graded using the Gleason system, in which tumors with higher Gleason Scores (GSs) tend to be more aggressive^{5,6}. However, the relationship between individual GS of clinically-localized PCs and those that progress to metastatic disease is poorly understood⁷. The tumorigenic events during PC progression have been difficult to investigate, and the ability to characterize late stages of PC progression is lacking due to very limited availability of metastatic tissues. In addition, 60-90% of PCs are multifocal⁸, in which one prostate contains several seemingly unconnected locations of cancer growth. The development of multifocal PC is still highly debated and two models have been described⁸. Either one initially-transformed cancer has spread to multiple locations within the prostate (monoclonal), and/or the cancer arises independently several times in the same gland (multiple subclones)⁹⁻¹⁸. The latter option indicates the possibility that aggressive and non-aggressive cancer foci co-exist in the same prostate gland, and is supported by the finding that individual foci of multifocal PC often present with unique GSs¹⁹. Consequently, the index lesion (the cancer lesion with the largest volume or the highest GS depending on the study) may not be representative of the PC pathology²⁰, and subsequently complicates sample selection for analysis. Therefore, previous studies that have not accounted for prostate tumor multifocality, or used only the index lesion, are potentially flawed.

Currently, no curative therapies are available for metastatic PC. Thus, a central dilemma in management of clinically localized PC is whether to postpone treatment until the disease becomes more aggressive in order to minimize patient health side effects, or to treat immediately to avoid dissemination. Identifying the primary lesion that drives cancer progression is critically advantageous in order to guide treatment decisions at the time of diagnosis. In this scenario, ideally, only patients with potential aggressive PC lesion(s) should undergo surgery, while patients with exclusively clinically insignificant (non-aggressive) cancer should be monitored using active surveillance.

DNA methylation alterations occur in every cancer type, and importantly, DNA methylation levels change concordantly with tumor aggressiveness in most types of cancer²¹. Epigenetic alterations can drive tumorigenesis and determine tumor aggressiveness, and therefore, can be used for PC diagnostic purposes²², as well as to inform on therapeutic approaches^{23,53}. Although PC has been shown to harbor a great hereditary element^{24,25}, only an estimated ˜30% of these factors have presently been accounted for²⁶. Interestingly, recent studies have been able to tie a connection between genetic alterations and DNA methylation changes, which indicate that DNA methylation changes hold information about clonal evolution of PC. For example, multiple metastases within a PC patient have been shown to arise from a single precursor cancer cell, or focus, by copy number alterations (CNA) as well as by DNA methylation changes^{27,28,54}, suggesting that only one focus of a multifocal PC is responsible for the development of the metastatic lesions. Moreover, unified evolution of DNA methylation and CNAs was identified in five cases of multifocal PC with monoclonal origin and their matched lymph node metastases¹¹.

### SUMMARY OF THE INVENTION

One aspect of the present invention is directed to a method of identifying subjects having prostate cancer. The method includes obtaining a sample from a subject, isolating DNA from the sample, determining the methylation status of the DNA and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The methylated probe includes a sequence region that extends up to 250 base pairs upstream and downstream from the methylated probe. The comparison indicates whether the sample is normal, a non-aggressive prostate cancer, or an aggressive prostate cancer.

Examples of methylation sensitive assays that can be used to determine the DNA methylation status include but are not limited to HM450, HM850, real-time methylation sensitive PCR (MSP), MethyLight and Pyrosequencing.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is to provide a method of classifying prostate cancer. The method includes obtaining a sample from a subject, isolating DNA from the sample, determining the methylation status of the DNA, and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The methylated probe includes a sequence region that extends up to 250 base pairs upstream and downstream from the methylated probe. The comparison indicates whether the prostate cancer is non-aggressive or aggressive.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is directed to a method of identifying subjects having prostate cancer. The method includes obtaining a sample from a subject, isolating DNA from the sample, determining the methylation status of the DNA and comparing the methylation status of the DNA to one or more methylated probes selected from the following: cg20399616, cg12737160, cg23624008, cg09789590, cg23095615, cg14273822, cg04294888, cg12098872, cg21981270, cg21526205, cg09806262, cg01940855, cg23855505, cg18423852, cg22260952, cg21883802, cg00036011, cg26573704, cg12727795 and cg01404317. The methylated probe includes a sequence region that extends up to 250 base pairs upstream and downstream from the methylated probe. The comparison indicates whether the sample is normal or prostate cancer.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is to provide a method of classifying prostate cancer. The method includes obtaining a sample from a subject, isolating DNA from the sample, determining the methylation status of the DNA, and comparing the methylation status of the DNA to one or more methylated probes selected from the following: cg25729795, cg09771468, cg01386424, cg21397588, cg18595258, cg01957088, cg12950441, cg05733231, cg19155518, cg13060157, cg16201038, cg01906055, cg16473141, cg17380661, cg04145287, cg22462983, cg01419991, cg06969287, cg04634417 and cg16692973. The methylated probe includes a sequence region that extends up to 250 base pairs upstream and downstream from the methylated probe. The comparison indicates whether the prostate cancer is non-aggressive or aggressive.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

In other aspects of the present invention, the methylated probes that are used in identifying subjects having prostate cancer and/or classifying prostate cancer can be one or more methylated probes selected from those methylated probes listed in Table 4. The sequences of all the probes listed in Table 4 are also available from Illumina.

Prostate Cancer (PC) is a slow-growing malignancy, but acquires aggressive traits in a subset of cases. PCs have a propensity to be multifocal. Here, we show that DNA methylation of PC metastases is highly similar to specific primary foci, build an aggressiveness classifier, and determined PC aggressiveness of foci based on DNA methylation profiles in matched primary tumors and metastases. The classifier is significant associated with aggressiveness and the presence of lymph node metastases (P=9.2×10⁻⁵) and invasive tumor stages (P=7.7×10⁻⁷) in an independent cohort. Overall, this invention provides molecular-based support for determining PC aggressiveness with importance for clinical decision-making.

Other aspects and advantages of the invention will be apparent from the following description and the appended claims.

### BRIEF DESCRIPTION OF THE DRAWINGS

FIGS. 1A-1B. Strategy and sample selection. (FIG. 1A) Schematic representation of a prostate gland with four cancer foci (green and orange areas) and a pelvic lymph node with metastasis marked by a purple star. We discovered that we can determine the primary focus of metastasis origin based on DNA methylation in the lymph node metastasis, and this in turn will represent the most aggressive cancer subclone. By determining the aggressive subclone in multifocal PCs, we will obtain groups of aggressive and non-aggressive samples, which will form the basis for developing a classifier to determine the aggressiveness of primary PC foci. (FIG. 1B) An overview of the samples from patient 41 is shown in the upper left corner. P=patient, T=primary tumor focus, NL=tumor-negative lymph node, and PL=tumor-positive lymph node. The physical location of the five prostate samples and the two lymph node samples collected are shown on schematics of the dissected prostate gland (middle) and the lymphatic system (lower left corner), respectively.

FIGS. 2A-2B. DNA methylation patterns of lymph node metastasis indicate the potential primary focus/foci of origin. Left: Unsupervised clustering and heatmaps of all the samples from patient 41 (FIG. 2A) and patient 54 (FIG. 2B) based on the top 1% most variably methylated probes between all samples except the PLs. Dendrograms are shown above the heatmaps and the color key to the right. Right: Copy number alterations in Patient 41 (FIG. 2A) and Patient 54 (FIG. 2B). In each plot the samples were ordered based on the unsupervised clustering from the heatmaps to the left. The numbers and letters on the left of the plot designate the chromosome numbers. To the right is shown the color key; red=chromosomal gain and blue=chromosomal loss.

FIGS. 3A-3B. Building an aggressiveness classifier. (FIG. 3A) Phylogenetic reconstruction showing clonal relationships in each patient based on all filtered HM450 probes. Averaged normal prostate and normal lymph node samples were used for each tree. Sample types are colored with black (normal and PIN), orange (aggressive primary tumor), green (non-aggressive primary tumor), yellow shaded (undecided primary tumor), and purple (lymph node metastasis). Below each tree the longest Euclidean distance between any two samples in the tree are denoted so as to serve as a reference between the different trees. (FIG. 3B) MDS plot based on 25-probe classifier generated by GLMnet of the samples used for the analysis. The samples are separated into 3 distinct groups without overlap.

FIGS. 4A-4C. Validation of the PC aggressiveness classifier. (FIG. 4A) Manhattan plot of the probabilities calculated for 100 randomly selected samples from the TCGA PC cohort. Color bar in the bottom of the plot designates the sample types determined by TCGA. Black=Adjacent normal prostate. Yellow=Primary PC. Purple=PC Metastasis. The black dotted line marks the probability threshold used. (FIG. 4B) Distribution of the prediction of TCGA normal and tumor samples. (FIG. 4C) Evaluation of correctly predicted samples based on the histological microscopic examinations performed by TCGA.

FIGS. 5A-5E. Clinical information for predicted TCGA groups. (FIG. 5A) Pre-operative PSA among the aggressive (n=215) and non-aggressive (n=64) groups. Welch two sample t-test=0.005. Tumor size represented by the average intermediate dimension in cm among the aggressive (n=87) and non-aggressive (n=25) groups. Welch two sample t-test=0.9428. Percentage of patients with lymph node metastases at the time of surgery among the aggressive (n=187) and non-aggressive (n=52) groups. Fisher's exact 2-tailed P(Yes)=9.2×10−5. Pathological T stage distribution among the aggressive (n=217) and non-aggressive (n=64) groups. Fisher's exact 2-tailed P: P(T2)=2.2×10⁻⁷, P(T3)=7.7×10⁻⁷, P(T4)=0.6969. GS distribution among the aggressive (n=217) and non-aggressive (n=64) groups. Fisher's exact 2-tailed P: P(GS 6)=0.0591, P(GS 7)=0.2539, P(GS 8-10)=0.0220. P-values<0.05 are marked by an asterisk. (FIG. 5B) Distribution of Gleason 3+4 and 4+3 tumors among the aggressive (n=96) and non-aggressive (n=34) groups. Fisher's exact 2-tailed P(3+4)=0.8424. P-values<0.05 are marked by an asterisk. Pathological T stage of Gleason 7 tumors among the aggressive (n=96) and non-aggressive (n=34) groups. Fisher's exact 2-tailed P: P(T2)=1.5×10−4, P(T3)=1.2×10−4, P(T4)=1. In FIG. 5C, FIG. 5D and FIG. 5E, Fisher's exact 2-tailed tests were used to calculate significance. P-values<0.05 were considered significant.

FIGS. 6A-6B. (FIG. 6A) Boxplot of 500 probes methylated in prostate tissue and unmethylated in peripheral blood. The normal lymph node samples had median DNA methylation beta values of ˜0.2. Any of the other samples with medians below 0.6 were excluded from further analysis: P15_PL and P32_PL.Tumor purity. (FIG. 6B) Boxplot of GSTP1 promoter DNA methylation for all samples. Tumor samples with median DNA methylation below 0.4 (dotted vertical line) were excluded from analysis: P17_T3 and P23_T3. The two PIN lesions were both just below 0.4 and were kept for further analysis.

FIG. 7. Sample dissimilarity. MDS plot drawn from Euclidean distances calculated based on all filtered probes.

FIG. 8. Unsupervised clustering and heatmaps. Clustering and heatmap visualization for all samples from Patients 14, 17, 23, 24, 26, 43, 52, 56, 84, 85, 88, and 98 was based on the top 1% most variably methylated probes between all samples, except the PLs. Dendrograms are shown above the heatmaps and the color key to the right.

FIG. 9. Input probes for GLMnet analysis. Heatmap of the top 3,000 most differentially methylated probes between the aggressive (n=31) and non-aggressive (n=10) groups based on the average methylation differences. Sidebar on the top shows the group of each sample (same as in FIGS. 3A-3B).

FIGS. 10A-10B. Clinical characteristics of TCGA PC GS 7 tumors. (FIG. 10A) Pathological T stage of Gleason 7 tumors among the aggressive (n=96) and non-aggressive (n=34) groups. Fisher's exact 2-tailed P: P(T2)=1.5×10⁻⁴, P(T3)=1.2×10⁻⁴, P(T4)=1. (FIG. 10B) Distribution of Gleason 3+4 and 4+3 tumors among the aggressive (n=96) and non-aggressive (n=34) groups. Fisher's exact 2-tailed P(3+4)=0.8424. P-values<0.05 are marked by an asterisk.

FIGS. 11A-11C. DNA methylation of metastasis and primary site from the same patient is highly similar. (FIG. 11A) Between-sample correlation plot. Sample names are shown to the left of the plot. At the top and the left of the plot are colored sidebars showing sample type and patient identifier. The sidebar to the right of the plot shows the correlation coefficient color key; red being high correlation and blue low correlation. P=patient, AN=adjacent normal, T=primary tumor focus, NL=tumor-negative lymph node, and PL=tumor-positive lymph node. (FIG. 11B) Enlargement of correlation amongst primary tumor foci in patient 41. (FIG. 11C) Enlargement of correlation between all primary tumor foci and all positive lymph nodes.

FIG. 12. Input probes for GLMnet analysis. Heatmap of the top 3,000 most differentially methylated probes between the aggressive (n=31) and non-aggressive (n=10) groups based on the average methylation differences. Sidebar on the top shows the group of each sample.

### DETAILED DESCRIPTION OF THE INVENTION

A “biomarker” as used herein refers to a molecular indicator that is associated with a particular pathological or physiological state. The “biomarker” as used herein is a molecular indicator for cancer, more specifically an indicator for prostate cancer.

As used herein the term “cancer” refers to or describes the physiological condition in mammals that is typically characterized by abnormal and uncontrolled cell division or cell growth. Examples of cancer include but are not limited to, carcinoma, lymphoma, blastoma, sarcoma, and leukemia.

As used herein, a “subject” is preferably a human, non-human primate, cow, horse, pig, sheep, goat, dog, cat, or rodent. In all embodiments, human subjects are preferred. The “subject” may be at risk of developing prostate cancer, may be suspected of having prostate cancer, or may have prostate cancer. In addition, a “subject” may simply be a person who wants to be screened for prostate cancer.

### Example 1

### DNA Methylation Patterns of Lymph Node Metastases Indicate the Potential Primary Focus/Foci of Origin

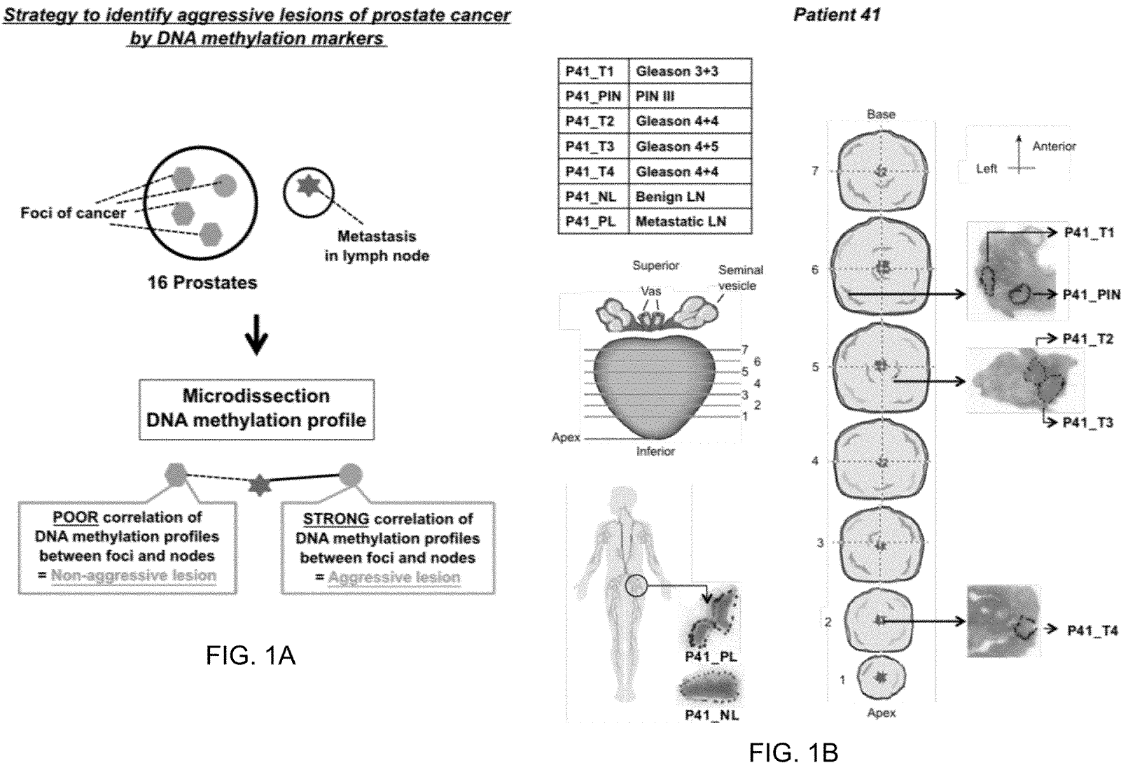

In this study, aggressive primary cancer focus/foci can be identified from multifocal PC by the degree of correlation of DNA methylation to lymph node metastases, which represent an aggressive trait (FIG. 1A). The study relies on four assumptions: 1) A subset of multifocal PCs arise from independent and sporadic genetic/epigenetic changes, effectively implying that distinct cancer foci develop through different molecular mechanisms/pathways and harbor unique proliferation, migration, and aggressiveness potential; 2) DNA methylation changes hold information about clonal evolution and will not change substantially upon dissemination^{11,28,29}, 3) metastases have the same clonal origin^{27,28}, and 4) The pelvic lymph nodes drain from a cancerous prostate and are likely the first location for development of metastases. Thus, lymph node metastases are herein defined as an aggressive trait and are used as an indicator of the ability to establish distant metastases as well.

We used the Illumina HumanMethylation450 BeadArray (HM450) platform to measure the DNA methylation status of matched primary tumors and pelvic lymph node metastases in 16 patients with multifocal disease (Table 1). The prostate and lymph node tissue samples stored in FFPE tissue blocks were sectioned, H&E stained (FIG. 1B), and examined by a trained pathologist. All areas of cancer were marked and given a GS. In addition, areas of adjacent normal prostate cells and prostatic intraepithelial neoplasia (PIN) were marked when possible, summing to a total of 92 samples (Methods).

TABLE 1
Patient Information

| | | Radical | | | | | Patient |
|---|---|---|---|---|---|---|---|
| Patient | Age | Prostatectomy | Tumor | Gleason | Nodal | Malignant | Status |
| no. | (years) | (year) | Stage | Score | Stage | LNs | (2015) |
| 14 | 64 | 2013 | pT3a | 4 + 5 | pN1 | 2/16 | Alive |
| 15 | 63 | 2007 | pT3a | 3 + 4 | pN1 | 2/9 | Alive |
| 17 | 61 | 2000 | pT3b | 3 + 5 | pN1 | 2/22 | Dead |
| 23 | 68 | 2004 | pT3 | 4 + 5 | pN1 | 14/49 | Dead |
| 24 | 66 | 2013 | pT3 | 3 + 4 | pN1 | 1/9 | Alive |
| 26 | 60 | 2013 | pT3b | 4 + 5 | pN1 | 3/45 | Alive |
| 32 | 66 | 2013 | pT3b | 4 + 4 | pN2 | 8/32 | Alive |
| 41 | 68 | 1999 | pT3b | 3 + 5 | pN1 | 6/11 | Dead |
| 43 | 62 | 2004 | pT3b | 5 + 4 | pN1 | 25/51 | Unknown |
| 52 | 75 | 2013 | pT3b | 5 + 4 | pN1 | 14/42 | Alive |
| 54 | 55 | 2008 | pT4 | 5 + 5 | pN1 | 4/8 | Dead |
| 56 | 57 | 2013 | pT3b | 5 + 4 | pN1 | 11/43 | Alive |
| 84 | 58 | 2008 | pT3b | 5 + 4 | pN1 | 4/50 | Alive |
| 85 | 61 | 2013 | pT3b | 4 + 4 | pN1 | 4/29 | Alive |
| 88 | 65 | 1991 | pT3b | 4 + 5 | pN2 | 5/20 | Dead |
| 98 | 79 | 2013 | pT3b | 4 + 5 | pN1 | 8/23 | Alive |

LN = lymph node

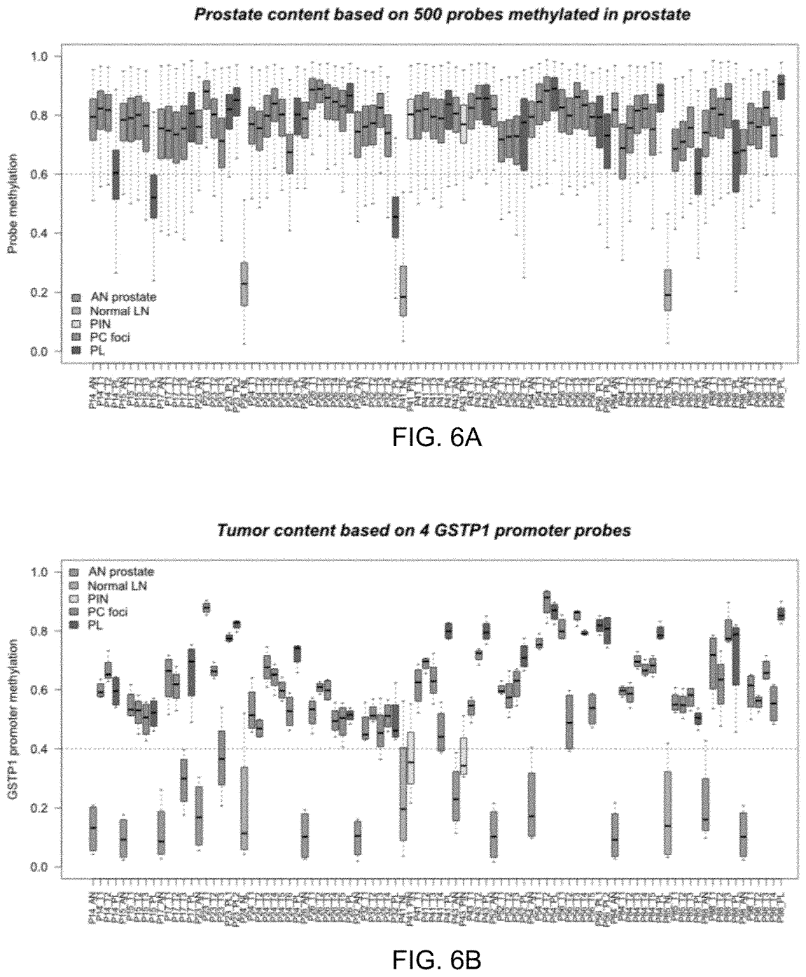

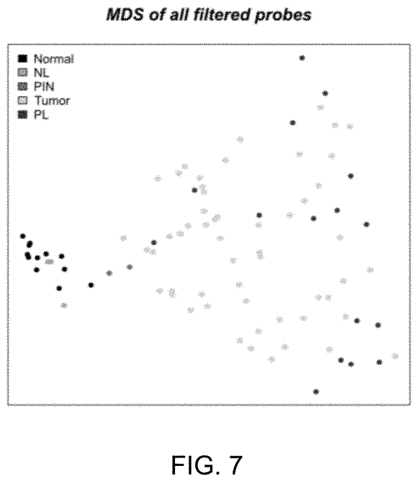

Sample purity was tested with respect to either infiltration of normal cells or leukocytes caused by inflammation (Methods, FIG. 6A-6B). Two primary tumor foci were removed due to low tumor cell content (P17_T3 and P23_T3), and two positive lymph node (PL) metastases were removed due to high leukocyte content (P15_PL and P32_PL), thereby excluding all samples from Patients 15 and 32. HM450 DNA methylation data from the remaining 14 patients were compared in a multidimensional scaling (MDS) plot, in which samples are placed in two-dimensional space based on dissimilarity (FIG. 7). Primary tumors and lymph node metastases were highly heterogeneous with no obvious subgroups, whereas normal prostate and lymph node tissues formed a tight cluster, as expected, indicating that the experimental protocol was successful.

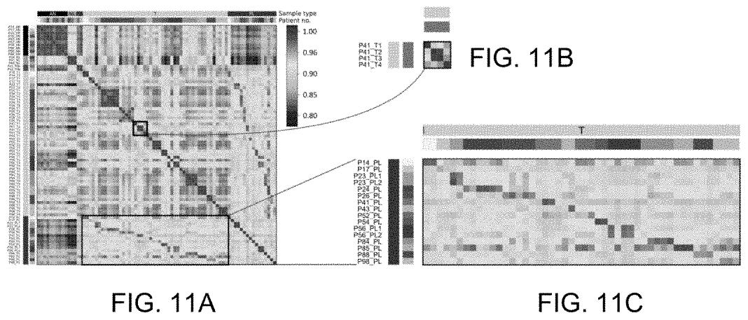

In order to investigate if DNA methylation patterns hold information about clonal evolution in PC, Pearson correlations amongst all the samples were calculated, plotted, and visualized using heatmaps (FIG. 11A). Firstly, primary foci from the same patient showed more variable correlation coefficients (0.89-0.99), as compared to for interpatient AN-AN samples (0.96-0.99) and interpatient AN-NL samples (0.90-0.94), indicating that multiple cancer subclones are present in some patients (FIG. 11B) and in turn may hold distinct tumorigenic potential. Secondly, lymph node metastases consistently showed the highest correlation to one or more of the primary tumor foci from the same patient (0.94-0.98, FIG. 11C). Thus, DNA methylation profiles had not diverged to such a degree that metastases and primary tumors remained comparable. Taken together, these results demonstrate that a subset of multifocal PCs show independent epigenetic changes, indicating that cancer foci develop from unique subclones. Furthermore, the DNA methylation profiles of lymph node metastases are highly correlative to a focus/foci from individual patients.

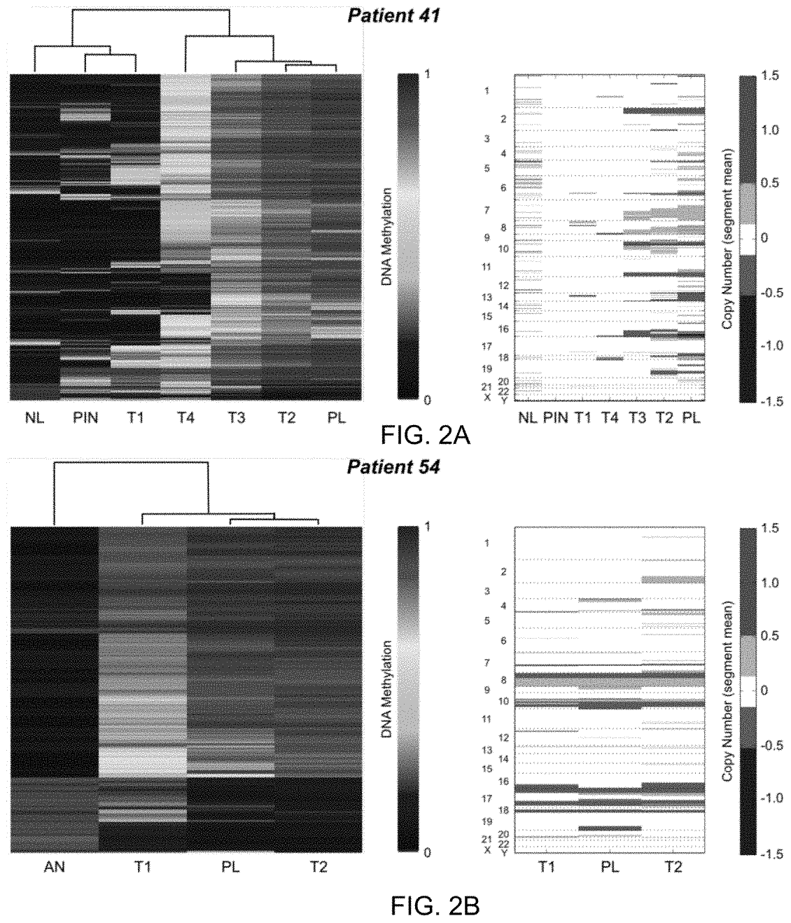

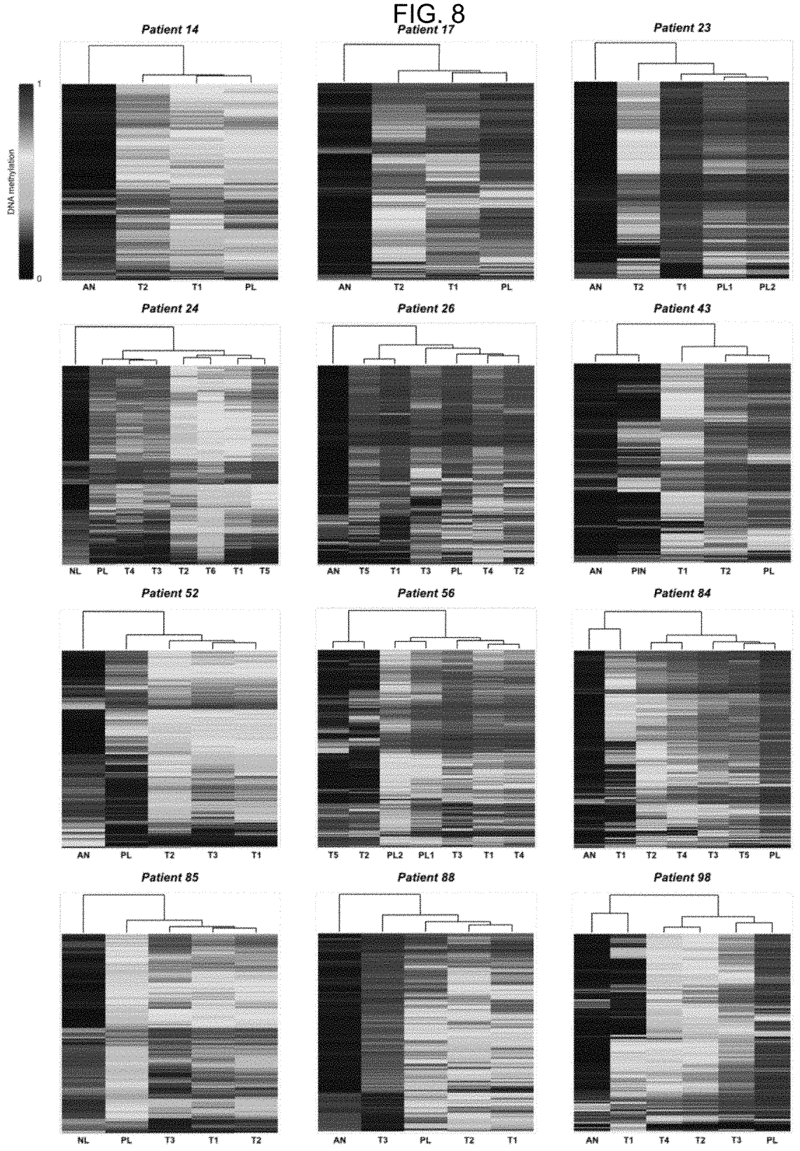

We first investigated the DNA methylation profiles of PC foci among individual patients. To identify the focus of origin of lymph node metastasis, we selected the top 1% most variably methylated probes between all samples for each patient, excluding PLs. The DNA methylation levels of these probes from all samples, including PLs, were then compared by unsupervised hierarchical clustering and heatmap visualization. Based on similar DNA methylation levels, PLs clustered with other samples, thereby providing information about the potential clonal relationship. Heatmaps after unsupervised clustering of these probes for two representative patients, Patients 41 and 54 (FIG. 2A and FIG. 2B, left panels), as well as for the remaining 12 patients with lymph node metastases (FIG. 8), showed PLs clustering with one or more of the primary tumor foci, whereas no PLs clustered with the adjacent normal prostate tissues, normal lymph nodes, or PIN lesions. In all 14 cases with lymph node metastases, the PLs clustered with one or more of the primary tumor foci, and no PLs clustered with the adjacent normal prostate tissues, normal lymph nodes, or PIN lesions (FIG. 2A, FIG. 2B and FIG. 8). In addition, PLs were found to cluster and were highly correlated in two patients (P23 and P56) with multiple PLs (0.99 and 0.98, respectively; FIG. 8), supporting the assumption (#3) that metastases have the same clonal origin.

The PL DNA methylation profile for Patient 41 was very similar to and clustered very closely with the T2 and T3 foci, while the T4 and T1 foci were more dissimilar, as shown by the dendrogram at the top of the heatmap. For this patient, the T2 and/or T3 foci are the most likely origin(s) of the metastasis. Furthermore, the physical juxtaposition of T2 and T3 in the prostate specimen (FIG. 1B) may suggest that they diverged from the same population of transformed cells during tumorigenesis. For Patient 54, which only had two primary foci, the PL DNA methylation was very similar to both the T1 and T2 foci. Hence, both patients displayed multiple primary tumor foci with very similar DNA methylation profiles, indicating a monoclonal origin of these foci. In addition, Patient 41 also displayed tumor foci with very different DNA methylation profiles, indicating the occurrence of multiple independent transformation events and therefore, multiple subclones.

In order to validate these findings, we took advantage of the recent evidence that the HM450 DNA methylation platform can also be used to determine CNAs by summing the methylated and unmethylated signal intensities of the probes^{30,31}. This analysis provided additional evidence that the T2 and T3 foci were very similar to the PL in Patient 41. All had deletions on chromosomes 2, 10, 11, and 16 and gains on chromosomes 7, 8, and 10, however, these regions were not altered in the T1 or T4 foci, which show different CNA patterns (FIG. 2A, right panel). All three samples from Patient 54 show multiple shared alterations, as well as deletion of the short arm and amplification of the long arm of chromosome 8, both common features of PC^{32,33} (FIG. 2B, right panel). Thus, the CNA analysis supports our findings of multiple subclonal origins in Patient 41 (FIG. 2A) and a monoclonal origin in Patient 54 (FIG. 2B) using DNA methylation analysis. Moreover, the CNA results also support our finding that the origin of lymph node metastasis can be determined by DNA methylation.

Similarly, all PLs clustered with one or more primary tumor foci from the remaining 12 cases using our DNA methylation-based approach (FIG. 8). Furthermore, nine patients (P23, P24, P26, P41, P43, P56, P84, P88, and P98) showed clearly distinct DNA methylation patterns among the primary foci, indicating the existence of independent tumor subclones. Taken together, these results suggest that the DNA methylation pattern of a lymph node metastasis can be used to identify the potential primary focus/foci of origin of metastasis, and that PC patients may contain subclones with aggressive and non-aggressive potential.

### Example 2

### Development of a Panel of DNA Methylation Markers as a Classifier for PC Aggressiveness

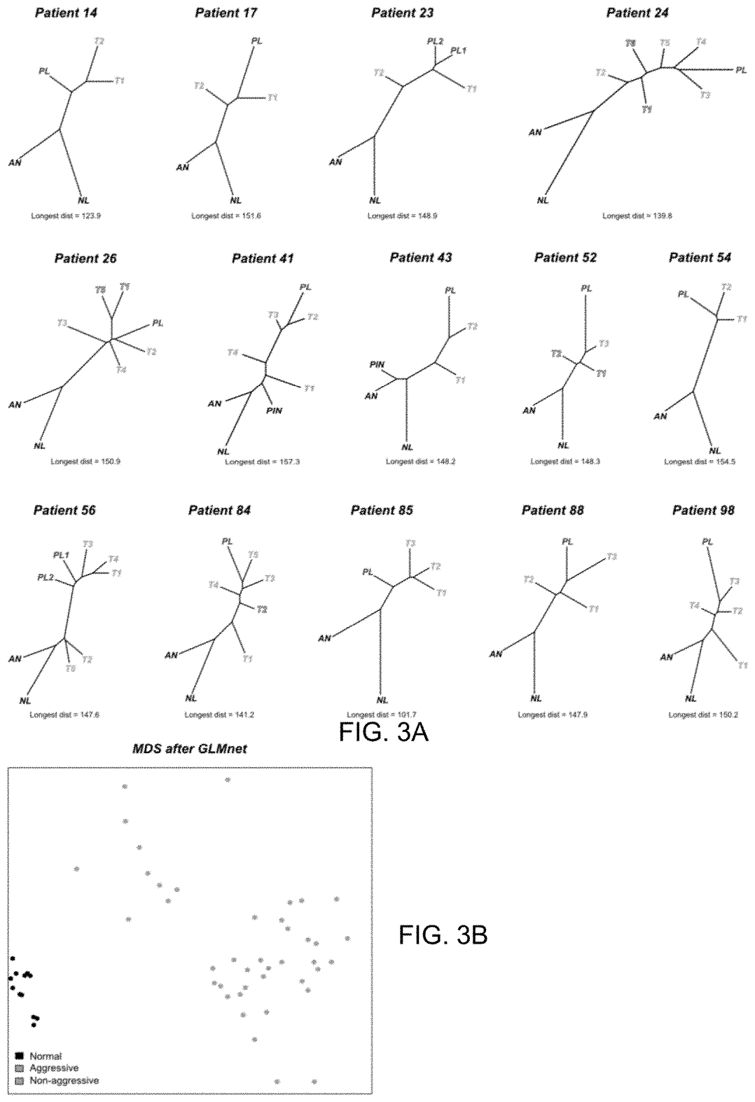

We next devised a DNA methylation-based PC aggressiveness classifier to categorize primary tumor foci as either aggressive or non-aggressive. The unsupervised hierarchical clustering approach effectively identifies the primary origin of lymph node metastases. However, in order to categorize the aggressiveness of individual foci in a quantitative, unbiased, and objective manner, we calculated Euclidean distances between any two samples within a patient using all filtered HM450 probes. Euclidean distance, like Pearson correlation, compares sample similarities, but maintains data variability, and is also superior for analysis of differential expression³⁴. We divided the scale of Euclidean distances into discrete categories (aggressive, undecided, and non-aggressive) for all primary tumor foci. Since the purpose of this categorization method is to assemble groups of genuinely aggressive and non-aggressive tumors for biomarker development, we included a gap of 10 Euclidean distance units (undecided category) to reduce the risk of misclassification. Sample categorization for each patient is shown using DNA methylation-based phylogenetic trees where samples are colored as a function of aggressiveness (FIG. 3A and overview in Table 2).

TABLE 2
PC Foci Aggressiveness by Patient

| | | No. of | No. of |
|---|---|---|---|
| Patient | Multiple | Aggressive | Non-Aggressive |
| No. | Subclones | Subclones | Subclones |
| 14 | No | 2 | 0 |
| 17 | No | 2 | 0 |
| 23 | Yes | 1 | 1 |
| 24 | Yes | 3 | 1 |
| 26 | Yes | 2 | 1 |
| 41 | Yes | 2 | 2 |
| 43 | Yes | 1 | 1 |
| 52 | ? | 1 | 0 |
| 54 | No | 2 | 0 |
| 56 | Yes | 3 | 2 |
| 84 | Yes | 3 | 1 |
| 85 | No | 3 | 0 |
| 88 | No | 3 | 0 |
| 98 | Yes | 3 | 1 |

Taken together, our developed categorization approach found that eight patients (Patients 23, 24, 26, 41, 43, 56, 84, and 98) showed independent DNA methylation profiles indicative of multiple subclones. Five patients (Patients 14, 17, 54, 85, and 88) showed similar DNA methylation patterns indicating a monoclonal origin, and one patient (Patient 52) was not categorized as either (FIG. 3A and Table 2). These findings are in agreement with the unsupervised clustering data (FIG. 2A, FIG. 2B and FIG. 8) with the exception of Patient 88, which did not show discrete subclones as indicated by the heatmap and dendrogram. In this patient, the top 1% most variably methylated probes were not representative of the potential clonal relationship.

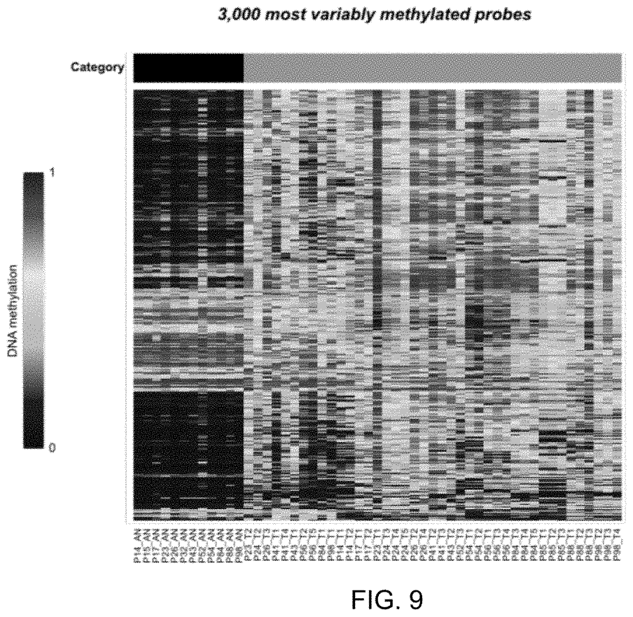

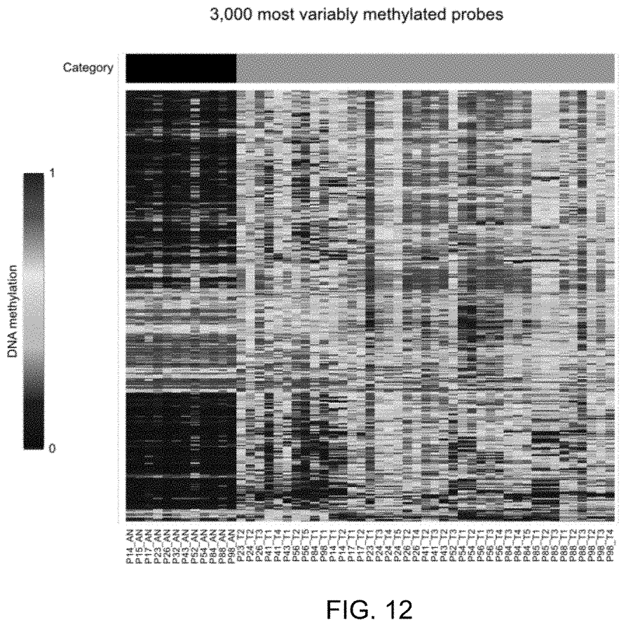

We next searched for differentially methylated probes between the aggressive and non-aggressive groups (FDR adjusted p<0.05), but found that the DNA methylation levels of no single probe were significantly different between the two groups. Using an FDR cutoff of 0.3, 231 probes were identified. Still, we continued to search for a set or panel of probes able to distinguish these groups from a larger panel. First, we generated a list of the 3,000 most differentially methylated probes between the assembled aggressive and non-aggressive groups based on mean DNA methylation differences (FIG. 9, FIG. 12, Table 4), which was subsequently used as input for the GLMnet algorithm35 along with information about normal, aggressive, and non-aggressive sample groups. The GLMnet model generates outputs in the form of probabilities of group membership, which are functions of the DNA methylation values for a given set of probes that differentiate the groups. For each sample, the probabilities of normal, aggressive, and non-aggressive groups sum to 1, and the group with the highest probability is the predicted phenotype of a given sample. Upon numerous iterations and refinement of the input probes list (Materials and Methods), we found a set of 25 probes (Table 3) that optimally predict normal, non-aggressive, and aggressive categories (FIG. 3B). Twenty one (84%) of the 25 probes in the classifier were among the probes with FDR-adjusted p<0.3 for either aggressive versus non-aggressive, aggressive versus normal, or non-aggressive versus normal comparisons.

TABLE 3
25-Probe Aggressiveness Classifier

| | | | UCSC | UCSC | RELATION |
|---|---|---|---|---|---|
| | | | REFGENE | REFGENE | TO UCSC |
| TargetID | Specificity | Methylation | NAME | GROUP | CPG ISLAND |
| cg00697992 | Normal | Hypo | NXPH2 | Body | N_Shore |
| cg01272707 | Aggressive | Hyper | — | — | Island |
| cg01819167 | Aggressive | Hypo | — | — | — |
| cg01906055 | Aggressive | Hypo | NCAPH | TSS1500 | N_Shore |
| cg02160684 | Aggressive | Hypo | TRIB1 | Body | S_Shelf |
| cg02560085 | Non-aggressive | Hyper | PCDHA1- | TSS200, Body | N_Shore |
| | | | PCDHA8 | | |
| cg03195164 | Non-aggressive | Hyper | C3orf37 | Body | S_Shore |
| cg03456213 | Non-aggressive | Hyper | C9orf3 | Body | Island |
| cg04634417 | Aggressive | Hypo | PCDHA1- | TSS200, Body | N_Shore |
| | | | PCDHA8 | | |
| cg06024295 | Aggressive | Hyper | CPN1 | 1stExon, | — |
| | | | | 5′UTR | |
| cg11748187 | Non-aggressive | Hyper | TCF7L2 | Body | S_Shore |
| cg13300630 | Aggressive | Hyper | ROBO1 | Body | — |
| cg13944838 | Non-aggressive | Hypo | GFPT2 | Body | Island |
| cg14399930 | Aggressive | Hyper | FBXO47 | TSS200 | — |
| cg15132013 | Aggressive | Hypo | SKI | Body | S_Shelf |
| cg16469740 | Aggressive | Hyper | HDAC9 | Body | — |
| cg17004353 | Aggressive | Hypo | CARS | 5′UTR, Body | — |
| cg17032646 | Aggressive | Hyper | SLC6A17 | TSS1500 | N_Shore |
| cg18315943 | Aggressive | Hypo | — | — | Island |
| cg19550524 | Aggressive | Hypo | — | — | — |
| cg20399616 | Normal | Hypo | BCAT1 | Body | Island |
| cg24517686 | Aggressive | Hypo | — | — | S_Shore |
| cg25961816 | Non-aggressive | Hypo | — | — | — |
| cg26447413 | Non-aggressive | Hyper | GAS1 | 1stExon | Island |
| cg27198013 | Non-aggressive | Hypo | RAI1 | 3′UTR | Island |

One of ordinary skill in the art will recognize that the methylated probe can also include sequence regions that extend upstream and/or downstream from the methylated probe. For example, the probe region could also include 250 bases (or greater) upstream and/or downstream from the probe, 200 bases upstream and/or downstream from the probe, 100 bases upstream and/or downstream from the probe, etc.

### Example 3

### The TCGA PC Cohort Validates the Potential of the Aggressiveness Classifier

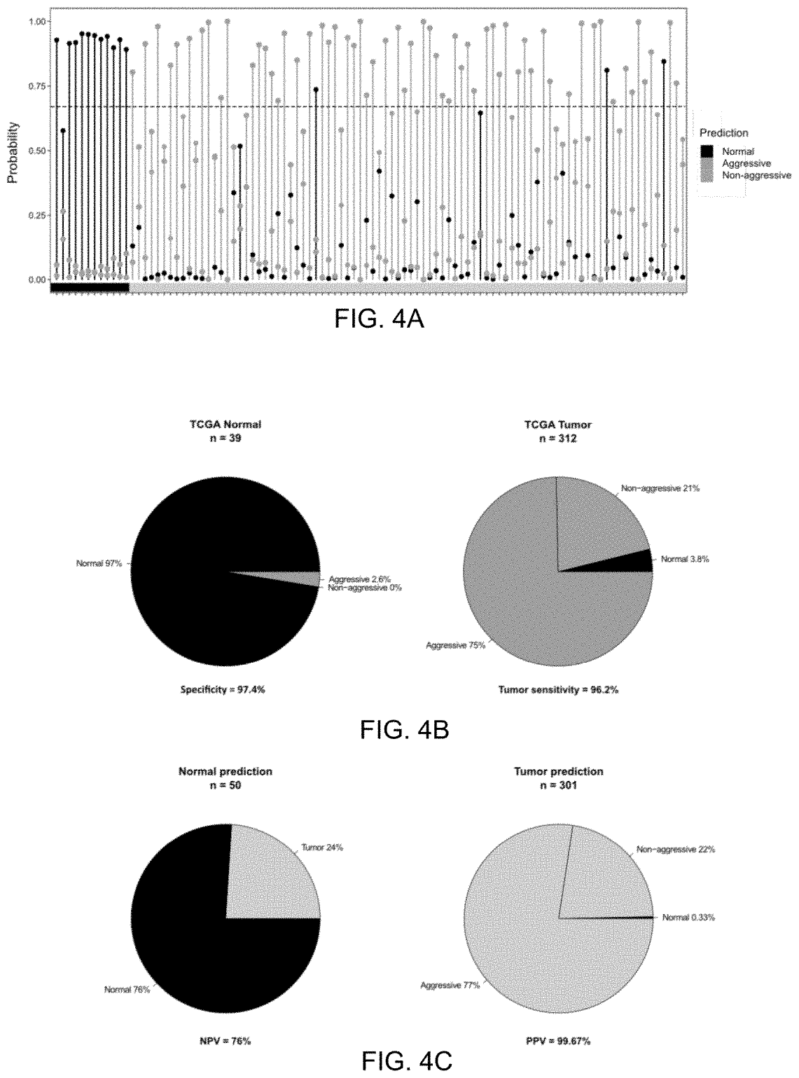

To test the classifier on an independent dataset, we took advantage of the publically available prostate adenocarcinoma HM450 DNA methylation data and accompanying clinical information from The Cancer Genome Atlas (TCGA) project. We tested 496 prostate samples (adjacent normal and tumor) using the classifier, and 70% of the samples (351 samples; 39 adjacent normal and 312 tumor samples) were predicted with a probability above 0.67 (FIG. 4A). Of the 39 normal TCGA samples, 38 were predicted as normal, and one as aggressive. Of the 312 tumors, 233 were predicted as aggressive, 67 were predicted as non-aggressive, and 12 were predicted as normal, resulting in a 97.4% specificity and a 96.2% sensitivity for PC as compared to adjacent normal tissue samples (FIG. 4B). Upon evaluation of the consistency between our predictions and the sample diagnoses (PC vs. AN) based on the histological microscopic examinations performed by TCGA, the classifier has a 76% negative predictive value (NPV) and a 99.7% positive predictive value (PPV; FIG. 4C).

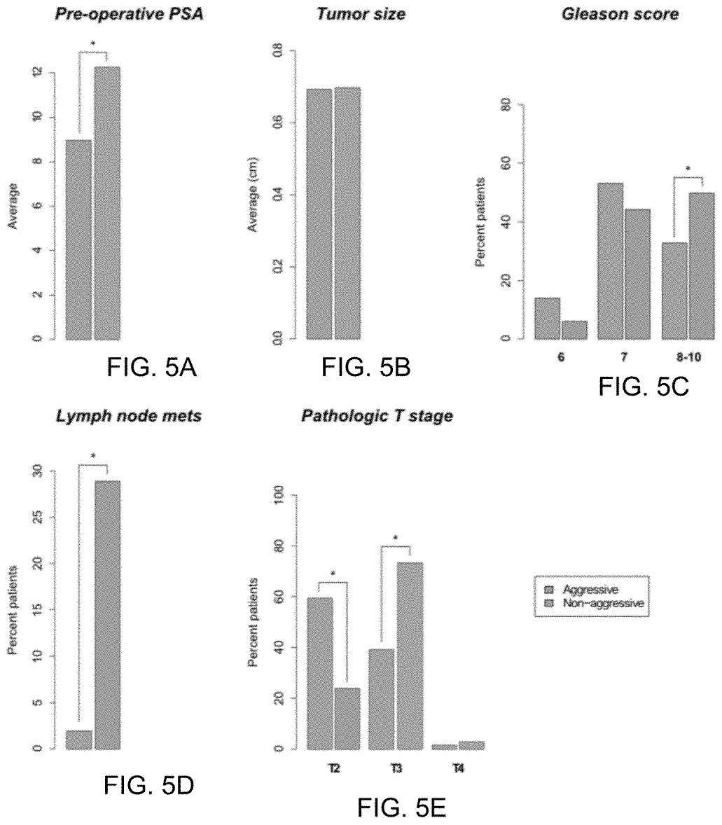

To evaluate the prognostic performance of the classifier, we consulted available clinicopathological co-variates known to be associated with PC aggressiveness (pre-operative PSA, tumor size, pathological GS, presence of lymph node metastases, and tumor stage) for samples with probabilities above 0.67. Aggressiveness was significantly (P<0.02) associated with the investigated co-variates except tumor size (FIG. 5A-5E). Pre-operative PSA levels were higher in the aggressive group compared to the non-aggressive group (P=0.005; FIG. 5A). However, similar tumor sizes between groups (FIG. 5B) indicates that aggressiveness and tumor size are independent as has also been suggested previously¹³.

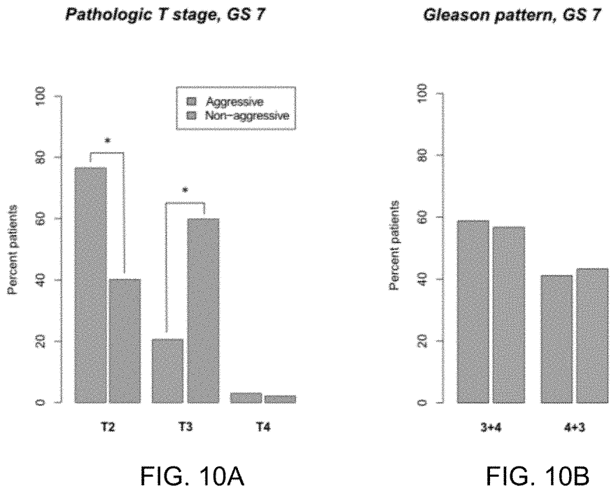

Upon further examination, tumors with high GSs (GS 8-10) were significantly associated with the aggressive group (P=0.022), but no such association was seen for tumors with low (GS 6) and intermediate (GS 7) scores (P=0.059 and P=0.254, respectively; FIG. 5A). GSs are well correlated with PC aggressiveness, especially at the low (GS 6) and high (GS 8-10) ends of the scale^{5,6}, and Gleason scoring is a valuable tool in PC treatment. However, additional information is required to determine aggressiveness for the intermediate (GS 7) tumors. Interestingly, the GS 7 tumors, which comprise nearly one-half of all TCGA PC tumors, were not significantly associated with non-aggressive or aggressive groups (FIG. 5A), indicating that this large group in particular may benefit from our DNA methylation-based classifier in order to determine whether active surveillance or ablative treatment is the best course of action. In support of this, we also found that the GS 7 tumors classified as non-aggressive were significantly associated with tumor stage T2 (P=1.5×10−4), while GS 7 tumors classified as aggressive were significantly associated with tumor stage T3 (P=1.2×10−4; FIG. 5B, FIG. 10A). Furthermore, we tested whether the primary and secondary patterns of the GS 7 tumors showed a correlation to the aggressive or non-aggressive groups (FIG. 5B, FIG. 10B). GS is calculated by summing the primary (largest pattern) and secondary (second largest pattern) Gleason grades, each of which ranges from 1 (well differentiated) to 5 (poorly differentiated)⁵. Interestingly, there was no difference in the distribution between 3+4 and 4+3 tumors and indicates that tumors of this large intermediate Gleason 7 group can be further and more accurately stratified using our molecular-based classifier to help determine whether active surveillance or ablative treatment should be performed.

Importantly, we found that significantly more patients classified as having an aggressive PC presented with lymph node metastases at the time of surgery as compared to patients with predicted non-aggressive tumors (P=9.2×10⁻⁵; FIG. 5D). Also, the pathological evaluation of tumor stage (FIG. 5E) showed significantly more organ-confined stage T2 tumors in the non-aggressive group (P=2.2×10⁻⁷) and significantly more of the capsule-penetrating and seminal vesicle invasive stage T3 tumors in the aggressive group (P=7.7×10⁻⁷). This strong correlation holds great promise for our classifier if developed into a molecular DNA methylation-based assay for needle biopsy samples, since the pathological tumor stage cannot be obtained until after surgery. Thus, if classification data had been obtained from needle biopsy samples prior to surgery, our recommendation would place ˜25% (67+12/312) of patients, the majority with GSs 6 and 7, in an active surveillance regimen rather than proceed with prostate ablation.

### Example 4

### Methods

### Patient Material, Samples, and Marking

Sixteen (16) patients diagnosed with multifocal PC having metastasized to one or more pelvic lymph nodes were enrolled in the study (Table 1). All patients had radical prostatectomies and removal of pelvic lymph nodes in the period between 1991-2013. No antiandrogen treatments were administered prior to surgery. The prostate and lymph node tissue samples were stored in FFPE tissue blocks. FFPE blocks were sectioned and H&E stained (FIG. 1B). A trained pathologist examined all slides covering the entire prostate and dissected lymph nodes, and all areas of cancer were marked and given a GS. In addition, areas of normal and PIN were marked when possible, adding up to a total of 92 samples. The marked H&E slides were used to guide the dissection of AN/PIN/PC cells from 8-10 unstained slides (5-10 μm).

### Deparafinization and Purification

The dissected tissue samples were deparaffinized using a double xylene wash followed by a double ethanol wash and drying of the pellets. For DNA extraction, the pellets were resuspended in 240 μl of PKD buffer and Proteinase K (Qiagen, miRNeasy FFPE kit (cat no. 217504)), and then incubated at 55° C. overnight and 85° C. for 15 min. After cooling the samples, 500 μl RBC buffer was added and the samples were run through gDNA Eliminator columns (RNeasy plus mini kit (cat no. 74134)) using RPE buffer to wash and EB buffer for elution.

### DNA Methylation Profiling

Genomic DNA (200-500 ng) from each FFPE sample was treated with sodium bisulfite and recovered using the Zymo EZ DNA methylation kit (Zymo Research, Irvine, Calif.) according to the manufacturer's specifications and eluted in 10 μl volume. An aliquot (1 μl) was removed for MethyLight-based quality control testing of bisulfite conversion completeness and the amount of bisulfite converted DNA available for the Infinium Methylation assay⁴². All samples that pass the QC tests were then repaired using the Illumina Restoration solution as described by the manufacturer. Each sample was then processed in the Infinium DNA methylation assay data production pipeline⁴³.

After the chemistry steps, BeadArrays were scanned and the raw signal intensities were extracted from the *.IDAT files using the R package methylumi. The intensities were corrected for background fluorescence and red-green dye-bias⁴⁴. The beta values were calculated as (M/(M+U)), in which M and U refer to the (pre-processed) mean methylated and unmethylated probe signal intensities, respectively. Measurements in which the fluorescent intensity was not statistically significantly above background signal (detection P-value>0.05) were removed from the data set. In addition, probes that overlap with known SNPs as well as repetitive elements were masked prior to data analyses. Specifically, all HM450 probes that overlapped with common SNPs with a minor allele frequency of greater than 1% (UCSC criteria) at the targeted CpG site, as well as probes with SNPs (MAF>1%) within 10 bp of the targeted CpG site were masked. HM450 probes that were within 15 bases of the CpG lying entirely within a repeat region were also masked prior to data analyses. The end result was a dataset of corrected beta-values for 396,020 probes which cover ˜21,000 genes.

### Calculation of Tumor Purity

To investigate the degree of leukocyte infiltration in each sample, public HM450 data from 96 male peripheral blood samples (GSE53740 and GSE51388) were downloaded using Marmal-aid⁴⁵. All HM450 probes with beta values>0.2 in male peripheral blood were excluded. The remaining probes were used to subset 500 probes that were hypermethylated in 43 normal prostate samples from TCGA, and thus hypomethylated in blood. Tissues of prostate origin from our study with mean DNA methylation of these probes below 0.6 were excluded from further analysis. Two lymph node metastases were excluded due to high blood content. Four GSTP1 HM450 probes (cg06928838, cg09038676, cg22224704, cg26250609) were used for tumor purity analysis as described in Brocks et al.¹¹. Primary tumors with mean DNA methylation below 0.4 were excluded from further analysis. Two tumor samples were excluded due to high normal content.

### Unsupervised Hierarchical Clustering

For each patient, probes with missing beta values (detection P-value>0.05) were excluded and the top 1% most variably methylated probes between all the samples except the PL(s) were selected. Heatmaps were used to display the DNA methylation levels and the unsupervised hierarchical clustering was performed with the hclust function in R (method=“complete”).

### Copy-Number Alteration Analysis

The CNAs were analyzed using the Champ package for R⁴⁶ using 28 adjacent normal prostate samples purified from FFPE tissues (12 from this study and 16 from own unpublished data) as a reference. Imported beta values were run through champ.norm and champ. CNA (filterXY=FALSE, batchCorrect=T, freqThreshold=0.3). The generated segment mean-files were intersected with the Infinium probe locations using BedTools and the resulting chromosomal loss and gain was illustrated in heatmaps using Matlab. Most of the samples showed noisy profiles, likely due to DNA breakage accumulated during the storage in FFPE, and the analysis could not be completed for all samples.

### PC Tumor Aggressiveness Categorization

Euclidean distances were calculated between any two samples using all 396,020 filtered probes. Averaged normal prostate and normal lymph node samples showed minimal variance and were used for the analysis. Normal prostate were considered to be very similar because only 0.65% (2561/396020) of standard deviations of all the probes were above 0.15. Normal lymph nodes were considered to be very similar because only 0.98% (3875/396020) of standard deviations of all the probes were above 0.15. The primary focus with the shortest Euclidean distance to the lymph node metastasis (T-PL dist 1) was categorized as aggressive. The additional distance to the other primary foci (T-PL dist 2; actual T-PL dist−T-PL dist 1=T-PL dist 2) were assessed in a density graph and a division of the scale based hereon. If T-PL dist 2s were only 0-10 units longer, they were also categorized as aggressive. This ensured that the foci of monoclonal origin would all be grouped as aggressive. Next, T-PL dist 2s longer by >20 units were categorized as non-aggressive origins and T-PL dist 2s of between 10-20 were categorized as undecided (overview in Table 2). In the two patients with 2 PLs the division of the primary tumors was done based on the PL with the shortest distance to a primary focus, which was P23_PL2 and P56_PL1.

### Phylogenetic Reconstruction

Methylation-based phylogenetic trees were inferred by the minimal evolution method⁴⁷. Euclidean distances were calculated using all 396,020 filtered probes.

### Calculation of Differential Methylation

Differential methylation between any two groups of samples were calculated using the champ.MVP( ) function from the ChAMP package utilizing either FDR<0.05 or FDR<0.3.

### Developing the DNA Methylation-Based PC Aggressiveness Classifier

By combining the categorized samples into groups of aggressive (n=31) and non-aggressive (n=10), we generated a list of 3,000 most variably methylated CpG sites (probes) between the groups. This list was used as input for the GLMnet algorithm³⁵, along with normal (n=12), non-aggressive (n=10), and aggressive (n=31) prostate sample groups. The GLMnet algorithm outputs a set of probes able to differentiate groups of samples based on their DNA methylation. Following 15 iterations, each output was evaluated by 1) the separation of the three groups (input as normal, aggressive, non-aggressive) in MDS plots like in FIG. 3B and FIG. 9; 2) the DNA methylation levels of the probes in heatmaps; 3) the prediction probabilities in Manhattan plots like in FIG. 4A for each set of probes run back on the input samples. This resulted in a refinement of the list of input probes from 3,000 to 39. Rerunning the GLMnet algorithm utilizing the 39 probes as input, a set of 25 probes (Table 3) was found to be the optimal predictor of our sample set according to normal, non-aggressive, and aggressive categories.

### Testing the Aggressiveness Classifier on TCGA DNA Methylation Data

The HM450 methylation data for the prostate adenocarcinoma samples from TCGA project was downloaded from the TCGA Data Portal (https://tcga-data.nci.nih.gov/tcga/). After filtering samples based on the same criteria as for our own samples, 499 samples (45 normal, 453 tumor, and 1 metastatic) remained. After removing samples with missing values among the 25 predictor probes, 496 samples remained (45 normal, 450 tumor, and 1 metastatic). The classifier was run on these samples and 70% were predicted with a probability above a cutoff of 0.67. A cutoff of 0.67 was chosen because as a consequence the probability for either of the two other groups must be 0.33 or less. Clinicopathological data was available for most samples in Biotab-files and are shown for the samples predicted above the 0.67 cutoff.

### Statistics

In FIG. 5A and FIG. 5B, Welch two sample t-tests were used to calculate statistical significance. In FIG. 5C, FIG. 5D and FIG. 5E, Fisher's exact 2-tailed tests were used to calculate significance. P-values<0.05 were considered significant.

### Data Access

Methylation array data have been submitted to the NCBI Gene Expression Obnibus (GEO; www.ncbi.nlm.nih.gov/geo/) under accession number GSE73549.

Identification of PC aggressiveness is fundamental to improving clinical decision-making in patients diagnosed with organ-confined PC regarding treatment or active surveillance. By implementing our design of examining DNA methylation in primary multifocal PC and matched lymph node metastases, we were able to examine the relationships amongst primary foci as well as the relationships between primary foci and metastases. Importantly, we found that more than half of the patients in our cohort showed multiple subclones, findings similar to previously reported studies^{9,11-14,16-18}, and also that DNA methylation of a lymph node metastasis is similar to a cancerous focus/foci from the same patient. Taking advantage of these findings, we developed a method to categorize the subclonal relationship and aggressiveness of individual PC foci. The resulting aggressive and non-aggressive sample groups, along with adjacent-normal samples, were used to search for biomarkers to distinguish the three groups, and the outcome was a 25-probe aggressiveness classifier. The classifier showed prognostic value when it was applied to samples from the PC cohort from TCGA.

For this study, we relied on the assumption that DNA methylation can inform on clonal evolution. Several studies have addressed the connection between DNA methylation and clonal evolution with high precision^{11,27,28} and recently, Costello and colleagues reported that phyloepigenetic relationships robustly recapitulate phylogenetic patterns in gliomas and their recurrences²⁹. Two or more foci originated from the same subclone in 11 of 14 patients in our cohort (FIG. 3A), indicating that an initial subclone seeded multiple locations through migration. We cannot definitively rule out that these are not actually one large or branched focus, since a fine physical connection can be hard to clearly distinguish in a pathological sample. Therefore, we do not attempt to determine which focus from the same subclone gave rise to the PL.

While clinical tools and techniques have improved immensely^{2,4,36-39}, the determination of tumor aggressiveness prior to physical manifestation must rely on biomarkers measured biochemically or at a molecular level. One impediment to success is how to define tumor aggressiveness with respect to a clinical end point. Often GS or time to PSA recurrence is used as a surrogate for PC aggressiveness, which would be more appropriately evaluated using metastatic progression or mortality. In this study, we used a novel approach in defining aggressiveness as the ability to give rise to lymph node metastases. The presence of lymph node metastases is an indication of tumor cells having acquired the ability to leave the primary site and proliferate in a secondary site, and thus acts as an indicator for the capacity of the cancer to establish distant metastases. In addition to this type of lymphatic dissemination, metastases can also arise through hematogenous dissemination to brain, lungs, liver, and bone marrow⁴⁸. Secondary cancer growths at these sites are not routinely removed during treatment for metastatic PC, and thus, the tissue for research is not available until postmortem. Although we recognize that distant metastases do not exclusively arise through lymphatic dissemination, we show that this clinical end point is very relevant alone or in concert with other clinicopathological parameters (FIG. 4A-4C, FIG. 5A-5E).

Gleason score 7 (GS 7) tumors are among the most difficult and poorly established backgrounds for making clinical decisions^{49,50}, however, our study demonstrated that aggressiveness of PCs with GS 7 using our classifier is highly correlated with pathological tumor stage, but not specific for primary or secondary Gleason patterns (4+3 or 3+4, FIG. 5B). Because of this, our classifier may challenge the current standard for clinical care, and may result in placing select PC patients into active surveillance and avoid invasive treatments.

A limitation to the presented study is that our discovery set is effectively only 14 patients, from whom we have 79 total samples. A larger discovery set would improve the study and would probably result in an enlargement of the classifier to more than 25 probes due to the vast PC heterogeneity⁵¹. Despite the modest size of the discovery set, we were able to validate the aggressiveness classifier, and thus, our study approach using publicly available TCGA prostate adenocarcinoma DNA methylation data from 496 primary tissues. Upon correlating our predictions with the TCGA clinicopathological information, we found a significant association (P<0.02) between aggressiveness and pre-operative PSA levels, pathological GS, presence of lymph node metastases and tumor stage, but interestingly, we did not find any correlation with tumor size. We do recognize that different clinical endpoints would be better suited to describe poor clinical outcome, however, but regret that the average follow-up period of the TCGA PRAD cohort was only 3.16 years. As a result, we found that too few patients had recurred and thus only found a significant difference between the groups for tumor status. Taken together, the presented data indicate the importance of using DNA methylation data to identify aggressive lesions more specifically than any currently used approach. This approach has clinical applications for early detection in PC biopsy specimens.

Upon suspicion of PC, prostate biopsies are performed as the standard-of-care method for PC diagnosis⁴⁰. Currently, prostate needle biopsies are most commonly performed trans-rectally in a systematic, yet random format. This systematic, random biopsy strategy has a high rate of misdiagnoses, since the non-targeted needles may either miss the clinically significant cancer focus, capture only a clinically insignificant cancer focus, or completely miss all cancer foci^{20,41}. Thus, the significant sampling error of traditional systematic, random prostate biopsies renders them unreliable for accurate characterization of index tumor location, volume, and GS⁴¹. The recently developed image-guided targeted prostate biopsy technique, which fuses magnetic resonance and three-dimensional transrectal ultrasound images, can reliably identify the location and the primary Gleason pattern of index lesions^{38,39}. By combining image-guided targeted biopsies and our DNA methylation classifier (following further clinical validation), the ability to identify aggressive focus, and subsequently characterize biopsy-detected PC foci more accurately should be enhanced. The ability to determine aggressiveness in a biopsy sample mapped to a particular prostate location should lead to the ability to make more informed clinical decisions regarding the choice between active surveillance of non-aggressive PC foci and surgery or targeted focal ablation therapy of the aggressive PC foci. Initially, the aggressiveness classifier should be developed into a more cost- and labor efficient test in the form of a custom DNA methylation array or multiplexed PCR-based assay (MSP or MethyLight)^{42,52}.

TABLE 4
Sequences of Probes

| | | | | | | INFIN- | |
|---|---|---|---|---|---|---|---|
| | | | | | | IUM_ | |
| SEQ | | | | RELATION_TO_ | | DE- | |
| ID | | UCSC_REFGENE_ | UCSC_REFGENE_ | UCSC_CPG | | SIGN_ | CHROMOSOME_ |
| NO: | TargetID | NAME | GROUP | ISLAND | ENHANCER | TYPE | 36 |
| 1 | cg00017221 | | | Island | NA | I | 1 |
| 2 | cg00018845 | | | Island | NA | II | 14 |
| 3 | cg00021256 | FEV | Body | N_Shore | TRUE | II | 2 |
| 4 | cg00036011 | HIST1H3G; HIST1H2 | TSS200; TSS1500 | N_Shore | NA | II | 6 |
| | | BI | | | | | |
| 5 | cg00046499 | GPR149 | Body | N_Shore | TRUE | II | 3 |
| 6 | cg00055529 | FBX047 | TSS200 | | NA | II | 17 |
| 7 | cg00063174 | WDR69 | TSS200 | Island | TRUE | I | 2 |
| 8 | cg00074145 | ONECUT1 | Body | N_Shore | NA | I | 15 |
| 9 | cg00083018 | | | N_Shore | NA | II | 6 |
| 10 | cg00087368 | SIM1 | Body | N_Shore | TRUE | II | 6 |
| 11 | cg00088183 | | | Island | NA | II | 1 |
| 12 | cg00107682 | | | Island | NA | II | 15 |
| 13 | cg00107772 | | | Island | NA | II | 14 |
| 14 | cg00114913 | LONRF3; LONRF3 | TSS1500; TSS1500 | Island | NA | I | X |
| 15 | cg00116265 | ZNF673; ZNF673; ZNF; | TSS1500; TSS1500 | N_Shore | NA | II | X |
| | | 63; ZNF673 | TSS1500; TSS150 | | | | |
| | | | 0 | | | | |
| 16 | cg00121904 | MAOB | TSS200 | Island | NA | II | X |
| 17 | cg00152008 | DMRTA2 | TSS200 | Island | NA | I | 1 |
| 18 | cg00153106 | | | Island | TRUE | II | 5 |
| 19 | cg00154357 | ZIC4; ZIC4; ZIC4; ZIC4 | Body; TSS1500; Bo | Island | NA | I | 3 |
| | | ; ZIC4 | dy; Body; TSS1500 | | | | |
| 20 | cg00154455 | ZNF517 | TSS1500 | N_Shore | NA | II | 8 |
| 21 | cg00155526 | NPR3 | Body | Island | NA | II | 5 |
| 22 | cg00158308 | OXGR1 | 5′UTR | N_Shore | TRUE | II | 13 |
| 23 | cg00170003 | NR2E1 | Body | Island | TRUE | II | 6 |
| 24 | cg00178984 | SLC16A7 | 5′UTR | | NA | I | 12 |
| 25 | cg00187981 | | | Island | NA | II | 8 |
| 26 | cg00188409 | | | N_Shore | TRUE | II | X |
| 27 | cg00192136 | DBXI | Body | Island | NA | II | 11 |
| 28 | cg00209038 | LHCGR | 1stExon | Island | TRUE | I | 2 |
| 29 | cg00210994 | C3orf26; FILIP1L; MIR | Body; Body; Body; 5 | N_Shore | NA | II | 3 |
| | | 548G; FILIP1L; FILIP1 | ′UTR; Body | | | | |
| | | L | | | | | |
| 30 | cg00224028 | SEMA3D | Body | | NA | II | 7 |
| 31 | cg00243661 | OR2D2 | 1stExon | | NA | II | 11 |
| 32 | cg00262621 | MMP23B | TSS1500 | Island | TRUE | II | 1 |
| 33 | cg00268840 | ALX4 | TSS1500 | Island | NA | I | 11 |
| 34 | cg00287829 | GRIN2A; GRIN2A; GR | TSS1500; 5′UTR; T | Island | NA | I | 16 |
| | | IN2A | SS200 | | | | |
| 35 | cg00296121 | FRMD4A | Body | N_Shore | NA | II | 10 |
| 36 | cg00326231 | CLTCL1; CLTCL1 | Body; Body | N_Shore | NA | II | 22 |
| 37 | cg00329697 | PTCH2 | 3′UTR | | TRUE | I | 1 |
| 38 | cg00332937 | | | Island | NA | I | 5 |
| 39 | cg00338801 | | | S_Shore | TRUE | II | 1 |
| 40 | cg00347563 | G1PC2; G1PC2 | 1stExon; 5′UTR | Island | NA | I | 1 |
| 41 | cg00348762 | DKFZP434H168; GNA | Body; Body; Body | S_Shore | NA | II | 16 |
| | | 01; GNA01 | | | | | |
| 42 | cg00352218 | | | N_Shore | NA | II | 6 |
| 43 | cg00360610 | SNRNP200 | Body | N_Shore | NA | II | 2 |
| 44 | cg00369477 | HTR2C; HTR2C | 5′UTR; 1stExon | Island | TRUE | II | X |
| 45 | cg00369811 | | | Island | TRUE | I | 6 |
| 46 | cg00374016 | | | Island | NA | II | 15 |
| 47 | cg00377070 | PLXNA4 | 3′UTR | | NA | II | 7 |
| 48 | cg00379648 | RFX4 | Body | Island | TRUE | I | 12 |
| 49 | cg00380985 | TCEAL6 | 5′UTR | N_Shore | NA | I | X |
| 50 | cg00400448 | | | N_Shore | TRUE | I | 15 |
| 51 | cg00407329 | SIM1 | Body | N_Shore | NA | II | 6 |
| 52 | cg00422120 | FEV | Body | N_Shore | TRUE | II | 2 |
| 53 | cg00423153 | SPHKAP; SPHKAP | TSS1500; TSS1500 | S_Shore | NA | I | 2 |
| 54 | cg00433861 | DOK6 | Body | Island | NA | II | 18 |
| 55 | cg00436476 | HIST1H4F | TSS200 | N_Shore | NA | I | 6 |
| 56 | cg00444390 | ORIOA6 | TSS200 | | NA | II | 11 |
| 57 | cg00463631 | GRIA2; GRIA2; GRIA2 | Body; Body; Body | Island | NA | I | 4 |
| 58 | cg00465476 | DUOX2; DUOX2 | TSS200; TSS200 | Island | NA | I | 15 |
| 59 | cg00466108 | DNAJC6 | Body | Island | TRUE | I | 1 |
| 60 | cg00474723 | ME3; ME3; ME3; ME3 | TSS200; TSS200; 1s | Island | NA | I | 11 |
| | | | tExon; 5′UTR | | | | |
| 61 | cg00478361 | | | Island | NA | II | 1 |
| 62 | cg00507135 | ACBD5; ACBD5; ACB | Body; Body; Body | N_Shore | NA | II | 10 |
| | | D5 | | | | | |
| 63 | cg00514609 | CRYAB; HSPB2 | TSS1500; TSS200 | | TRUE | I | 11 |
| 64 | cg00527440 | THAP4; THAP4 | Body; Body | Island | NA | II | 2 |
| 65 | cg00538458 | | | Island | NA | II | 16 |
| 66 | cg00554969 | TBX22; TBX22; TBX2 | Body; 5′UTR; Body | | NA | I | X |
| | | 2 | | | | | |
| 67 | cg00574740 | LMO7 | Body | Island | NA | II | 13 |
| 68 | cg00594129 | EBF1 | Body | Island | TRUE | II | 5 |
| 69 | cg00610991 | SLC8A1 | 5′UTR | N_Shore | NA | II | 2 |
| 70 | cg00613562 | SULF1; SULF1; SULF1 | 5′UTR; 1stExon; 1st | | TRUE | II | 8 |
| | | ; SULF1 | Exon; 5′UTR | | | | |
| 71 | cg00619126 | | | Island | NA | I | 2 |
| 72 | cg00625334 | C18orf34; C18orf34 | TSS200; TSS1500 | Island | NA | I | 18 |
| 73 | cg00626702 | FAM53A | Body | S_Shore | TRUE | I | 4 |
| 74 | cg00641409 | FLJ22536 | Body | Island | NA | I | 6 |
| 75 | cg00646019 | | | Island | NA | I | 15 |
| 76 | cg00653387 | PTN; PTN | 5′UTR; 1stExon | | NA | II | 7 |
| 77 | cg00663077 | ZFP42 | TSS1500 | Island | NA | I | 4 |
| 78 | cg00667647 | | | N_Shore | TRUE | II | 1 |
| 79 | cg00675569 | ALOX5 | Body | Island | TRUE | I | 10 |
| 80 | cg00688681 | | | Island | NA | I | X |
| 81 | cg00697992 | NXPH2 | Body | N_Shore | TRUE | II | 2 |
| 82 | cg00702719 | | | Island | NA | II | 6 |
| 83 | cg00739582 | MIR888; MIR890 | TSS1500; TSS1500 | | NA | II | X |
| 84 | cg00741609 | TLX1 | Body | Island | TRUE | II | 10 |
| 85 | cg00741836 | DOK5 | TSS200 | Island | NA | I | 20 |
| 86 | cg00744866 | CLASP2 | Body | | TRUE | II | 3 |
| 87 | cg00748427 | | | | TRUE | II | 14 |
| 88 | cg00753399 | MOBKL1B | Body | N_Shore | NA | II | 2 |
| 89 | cg00760950 | | | | TRUE | II | 13 |
| 90 | cg00765312 | ALX1 | Body | Island | NA | I | 12 |
| 91 | cg00766845 | NID2 | TSS200 | Island | NA | II | 14 |
| 92 | cg00767395 | | | N_Shore | NA | II | 2 |
| 93 | cg00772407 | DUSP6; DUSP6 | Body; Body | N_Shore | NA | II | 12 |
| 94 | cg00789198 | | | | NA | II | 19 |
| 95 | cg00802478 | RBM47 | 5′UTR | | TRUE | II | 4 |
| 96 | cg00817100 | | | Island | NA | I | 1 |
| 97 | cg00822361 | SSR3 | Body | N_Shore | NA | II | 3 |
| 98 | cg00838898 | | | N_Shore | TRUE | II | 16 |
| 99 | cg00839579 | MEOX2 | TSS1500 | | TRUE | I | 7 |
| 100 | cg00840332 | LEP | TSS200 | Island | NA | I | 7 |
| 101 | cg00850552 | SUMO3 | Body | N_Shore | NA | II | 21 |
| 102 | cg00854166 | EPO | Body | S_Shore | TRUE | II | 7 |
| 103 | cg00862116 | ABCB1 | 5′UTR | S_Shore | NA | II | 7 |
| 104 | cg00864458 | RPS20; RPS20 | Body; 3′UTR | N_Shore | NA | II | 8 |
| 105 | cg00875272 | TAL1; TAL1 | 5′UTR; 1stExon | N_Shore | NA | II | 1 |
| 106 | cg00880452 | SYCN | 1stExon | Island | NA | I | 19 |
| 107 | cg00884221 | IGFBP7 | TSS1500 | Island | NA | II | 4 |
| 108 | cg00893471 | HOXD3 | 5′UTR | Island | NA | II | 2 |
| 109 | cg00910015 | | | | NA | I | 2 |
| 110 | cg00918522 | | | N_Shelf | TRUE | II | 4 |
| 111 | cg00921266 | HOXA3; HOXA3; HO | 5′UTR; 5′UTR; TSS | N_Shore | NA | I | 7 |
| | | XA3 | 200 | | | | |
| 112 | cg00926215 | PAX3; PAX3; PAX3; C | Body; Body; Body; T | N_Shore | NA | I | 2 |
| | | CDC140; PAX3; PAX3; | SS1500; Body; Bod | | | | |
| | | PAX3; PAX3; PAX3 | y; Body; Body; Body | | | | |
| 113 | cg00928167 | | | Island | TRUE | II | 14 |
| 114 | cg00930873 | ALDH1A2; ALDH1A2 | TSS200; TSS200 | Island | TRUE | II | 15 |
| 115 | cg00932276 | EGFL6; EGFL6; EGFL | 5′UTR; 1stExon; 5′U | Island | TRUE | II | X |
| 116 | cg00944193 | NBEA | Body | Island | NA | I | 13 |
| 117 | cg00947032 | SPG20; SPG20; SPG20; | 5′UTR; 5′UTR; 5′UT | Island | NA | II | 13 |
| | | SPG20 | R; 5′UTR | | | | |
| 118 | cg00963378 | GJA3 | Body | Island | NA | I | 13 |
| 119 | cg00971050 | PTRF | Body | Island | NA | I | 17 |
| 120 | cg00980904 | RASSF1; RASSF1; RA | Body; Body; Body; 5 | N_Shore | NA | II | 3 |
| | | SSF1; RASSF1 | ′UTR | | | | |
| 121 | cg00990022 | NT5C1A | TSS1500 | Island | TRUE | II | 1 |
| 122 | cg00996827 | | | | NA | I | 1 |
| 123 | cg01016662 | | | Island | TRUE | I | 1 |
| 124 | cg01019587 | | | Island | NA | II | 7 |
| 125 | cg01025095 | | | | NA | I | 5 |
| 126 | cg01035198 | C6orf141; C6orf141 | 1stExon; 3′UTR | S_Shore | TRUE | II | 6 |
| 127 | cg01042334 | OPLAH | 5′UTR | Island | TRUE | II | 8 |
| 128 | cg01042641 | IFT140 | Body | S_Shore | NA | I | 16 |
| 129 | cg01056242 | GPR133 | Body | N_Shore | NA | I | 12 |
| 130 | cg01063813 | STAT6; STAT6 | 1stExon; 5′UTR | | TRUE | II | 12 |
| 131 | cg01065210 | RER1 | 3′UTR | S_Shore | NA | II | 1 |
| 132 | cg01068023 | | | N_Shore | NA | II | 1 |
| 133 | cg01085837 | | | Island | NA | II | 12 |
| 134 | cg01088410 | | | Island | NA | I | 5 |
| 135 | cg01098812 | | | Island | TRUE | II | 14 |
| 136 | cg01120165 | HDAC10; HDAC10 | TSS1500; TSS1500 | Island | NA | I | 22 |
| 137 | cg01128642 | OSR1 | TSS1500 | S_Shore | NA | I | 2 |
| 138 | cg01135546 | | | Island | NA | I | 17 |
| 139 | cg01138867 | GABRB2; GABRB2 | TSS1500; TSS1500 | S_Shore | NA | I | 5 |
| 140 | cg01167755 | NRG2; NRG2; NRG2; N | Body; Body; Body; | S_Shore | NA | I | 5 |
| | | RG2 | Body | | | | |
| 141 | cg01175020 | TLX1 | Body | Island | NA | I | 10 |
| 142 | cg01186595 | CHORDC1; CHORDC | Body; Body | N_Shore | NA | II | 11 |
| | | 1 | | | | | |
| 143 | cg01203385 | C2orf43 | TSS200 | Island | NA | I | 2 |
| 144 | cg01209023 | FTH1 | Body | N_Shore | NA | II | 11 |
| 145 | cg01218619 | | | Island | TRUE | I | 4 |
| 146 | cg01221209 | | | | NA | I | 5 |
| 147 | cg01245966 | ADCY8 | TSS1500 | Island | NA | II | 8 |
| 148 | cg01258751 | SIMI | TSS1500 | N_Shore | TRUE | II | 6 |
| 149 | cg01272707 | | | Island | NA | I | 6 |
| 150 | cg01291336 | SMARCD3; SMARCD | Body; Body | S_Shore | TRUE | II | 7 |
| | | 3 | | | | | |
| 151 | cg01308827 | ETV2 | TSS200 | N_Shore | NA | I | 19 |
| 152 | cg01309153 | SURF1; SURF2 | TSS1500; Body | S_Shore | NA | II | 9 |
| 153 | cg01348733 | SLFN11; SLFN11; SLF | TSS200; TSS200; T | Island | TRUE | I | 17 |
| | | N11; SLFN11; SLFN11 | SS200; TSS200; TS | | | | |
| | | | S200 | | | | |
| 154 | cg01384143 | C3orf14 | TSS1500 | Island | NA | I | 3 |
| 155 | cg01386424 | | | Island | NA | II | 6 |
| 156 | cg01386978 | PARK2; PARK2; PAR | Body; Body; Body | S_Shore | NA | I | 6 |
| | | K2 | | | | | |
| 157 | cg01404317 | SPG20; SPG20; SPG20; | 5′UTR; 5′UTR; 5′UT | Island | NA | II | 13 |
| | | SPG20 | R; 5′UTR | | | | |
| 158 | cg01410316 | ADAP 1 | Body | N_Shore | NA | II | 7 |
| 159 | cg01413054 | CAB39; CAB39; CAB3 | Body; Body; Body | | NA | II | 2 |
| | | 9 | | | | | |
| 160 | cg01419831 | | | Island | TRUE | I | 2 |
| 161 | cg01419991 | TRIB1 | 3′UTR | | NA | II | 8 |
| 162 | cg01422797 | PRDM13 | TSS1500 | N_Shore | NA | II | 6 |
| 163 | cg01439876 | | | Island | TRUE | I | 1 |
| 164 | cg01443071 | CNP | Body | Island | NA | I | 17 |
| 165 | cg01476568 | C10orf18 | 5′UTR | | TRUE | I | 10 |
| 166 | cg01481159 | | | Island | NA | II | 20 |
| 167 | cg01496199 | | | Island | TRUE | I | 19 |
| 168 | cg01498231 | SPOCK2; SPOCK2 | Body; Body | Island | NA | I | 10 |
| 169 | cg01498900 | ACOT2 | TSS200 | N_Shore | NA | II | 14 |
| 170 | cg01503299 | NOP14 | Body | N_Shore | NA | I | 4 |
| 171 | cg01505767 | LOC284798; LOC2847 | Body; TSS1500; Bo | Island | TRUE | I | 20 |
| | | 98; LOC284798 | dy | | | | |
| 172 | cg01520122 | CCL7 | TSS1500 | | NA | II | 17 |
| 173 | cg01521258 | CACHD1 | Body | S_Shore | NA | II | 1 |
| 174 | cg01533258 | GSC | Body | Island | TRUE | I | 14 |
| 175 | cg01546378 | GALM | TSS200 | | NA | II | 2 |
| 176 | cg01548300 | | | Island | NA | II | 5 |
| 177 | cg01563704 | HIST1H3G; HIST1H2 | TSS1500; TSS1500 | N_Shore | NA | II | 6 |
| | | BI | | | | | |
| 178 | cg01564165 | | | | NA | II | 13 |
| 179 | cg01587682 | PAX6; PAX6; PAX6 | Body; Body; Body | Island | TRUE | II | 11 |
| 180 | cg01589129 | | | Island | TRUE | I | 4 |
| 181 | cg01615475 | | | Island | NA | I | 11 |
| 182 | cg01625087 | | | N_Shore | NA | I | 6 |
| 183 | cg01635555 | | | S_Shore | NA | II | 16 |
| 184 | cg01657186 | NINJ2 | Body | S_Shore | TRUE | I | 12 |
| 185 | cg01661350 | KLHL1; KLHL1; ATX | 1stExon; 5′UTR; Bo | Island | TRUE | II | 13 |
| | | N8OS | dy | | | | |
| 186 | cg01663018 | | | Island | NA | I | 15 |
| 187 | cg01697719 | TBX1; TBX1; TBX1 | Body; Body; Body | Island | TRUE | I | 22 |
| 188 | cg01709619 | | | S_Shore | NA | I | 3 |
| 189 | cg01718742 | CDH8 | TSS200 | Island | NA | I | 16 |
| 190 | cg01735384 | | | Island | NA | I | 6 |
| 191 | cg01737550 | TCEAL6 | 3′UTR | N_Shelf | NA | II | X |
| 192 | cg01738650 | | | | TRUE | II | 6 |
| 193 | cg01751605 | PTN | TSS200 | | NA | II | 7 |
| 194 | cg01754780 | SRC1N1 | Body | Island | TRUE | I | 17 |
| 195 | cg01758418 | SLC7A3; SLC7A3 | TSS1500; TSS1500 | S_Shore | NA | II | X |
| 196 | cg01758512 | FUT9; FUT9 | 5′UTR; 1stExon | Island | NA | I | 6 |
| 197 | cg01759597 | DLX6AS | Body | | NA | I | 7 |
| 198 | cg01765758 | | | Island | NA | II | X |
| 199 | cg01765882 | LOC116437 | Body | | NA | I | 12 |
| 200 | cg01779765 | FLJ43390 | TSS1500 | Island | NA | I | 14 |
| 201 | cg01789478 | NEUROD1 | TSS200 | N_Shelf | TRUE | I | 2 |
| 202 | cg01796225 | CHORDC1; CHORDC | Body; Body | N_Shore | NA | II | 11 |
| | | 1 | | | | | |
| 203 | cg01804193 | | | Island | TRUE | II | 12 |
| 204 | cg01805282 | EYA4; EYA4; EYA4 | 5′UTR; 5′UTR; 5′UT | Island | TRUE | II | 6 |
| | | | R | | | | |
| 205 | cg01810111 | | | | TRUE | II | 6 |
| 206 | cg01814945 | HLA-J; NCRNA00171 | Body; Body | Island | TRUE | I | 6 |
| 207 | cg01816515 | C2orf43 | 5′UTR | Island | NA | II | 2 |
| 208 | cg01819167 | | | | NA | II | 12 |
| 209 | cg01819759 | RNF219 | TSS1500 | S_Shore | NA | II | 13 |
| 210 | cg01832005 | C3orf14 | TSS1500 | Island | NA | II | 3 |
| 211 | cg01832036 | DLX4; DLX4; DLX4 | Body; 5′UTR; 1stEx | Island | NA | I | 17 |
| | | | on | | | | |
| 212 | cg01837719 | FUT9 | TSS1500 | N_Shore | NA | II | 6 |
| 213 | cg01841641 | | | N_Shore | NA | I | 2 |
| 214 | cg01842807 | CYP2W1 | Body | Island | NA | I | 7 |
| 215 | cg01851874 | RUNX1T1; RUNX1T1; | Body; 5′UTR; 1stEx | | TRUE | I | 8 |
| | | RUNX1T1; RUNX1T1 | on; 5′UTR | | | | |
| 216 | cg01866955 | | | Island | NA | I | 6 |
| 217 | cg01871127 | | | N_Shore | NA | II | 2 |
| 218 | cg01872216 | | | Island | TRUE | I | 14 |
| 219 | cg01880908 | TNRC6C; TNRC6C | 3′UTR; 3′UTR | Island | NA | I | 17 |
| 220 | cg01883966 | STK19; STK19; STK19 | TSS200; TSS1500; | N_Shore | NA | II | 6 |
| | | ; DOM3Z | TSS1500; Body | | | | |
| 221 | cg01906055 | NCAPH | TSS1500 | N_Shore | NA | II | 2 |
| 222 | cg01912955 | SIM1 | Body | Island | NA | II | 6 |
| 223 | cg01913196 | DCAF12L1 | TSS200 | S_Shore | NA | I | X |
| 224 | cg01916115 | LAMA2; LAMA2; LA | 5′UTR; 1stExon; 1st | | TRUE | II | 6 |
| | | MA2; LAMA2 | Exon; 5′UTR | | | | |
| 225 | cg01932956 | | | Island | NA | I | 16 |
| 226 | cg01940855 | CHST11 | Body | Island | NA | I | 12 |
| 227 | cg01942318 | | | | TRUE | II | 2 |
| 228 | cg01957088 | HAS2; HAS2AS | 5′UTR; TSS200 | N_Shore | NA | II | 8 |
| 229 | cg01958086 | MEIS2; MEIS2; MEIS2 | Body; Body; Body; | N_Shore | NA | II | 15 |
| | | ; MEIS2; MEIS2; MEIS | Body; Body Body; | | | | |
| | | 2; MEIS2 | Body | | | | |
| 230 | cg01968493 | ZNF804A | TSS1500 | N_Shore | NA | II | 2 |
| 231 | cg01972576 | GML; GML | 5′UTR; 1stExon | Island | NA | I | 8 |
| 232 | cg01978237 | SIM1 | Body | N_Shore | NA | II | 6 |
| 233 | cg01981601 | TOX | TSS1500 | Island | NA | I | 8 |
| 234 | cg01984743 | STC2 | Body | N_Shelf | TRUE | I | 5 |
| 235 | cg01987516 | | | Island | NA | II | 10 |
| 236 | cg01996723 | | | N_Shore | NA | II | X |
| 237 | cg01997272 | | | Island | TRUE | I | 14 |
| 238 | cg01999333 | CASP14 | TSS1500 | | NA | II | 19 |
| 239 | cg02009088 | NRG2; NRG2; NRG2; N | Body; Body; Body; | Island | NA | I | 5 |
| | | RG2 | Body | | | | |
| 240 | cg02025583 | TMEM220 | Body | Island | NA | II | 17 |
| 241 | cg02026062 | FAM162B | Body | Island | NA | I | 6 |
| 242 | cg02029926 | CXCL1 | TSS1500 | N_Shore | NA | II | 4 |
| 243 | cg02053896 | SLC7A3; SLC7A3 | TSS200; TSS200 | S_Shore | NA | II | X |
| 244 | cg02056062 | WWTR1; WWTR1; W | Body; Body; Body | Island | NA | II | 3 |
| | | WTR1 | | | | | |
| 245 | cg02058408 | | | Island | NA | I | 17 |
| 246 | cg02078690 | BSX | TSS200 | Island | TRUE | I | 11 |
| 247 | cg02081266 | | | Island | TRUE | I | 17 |
| 248 | cg02086467 | OTX2 | 5′UTR | Island | NA | I | 14 |
| 249 | cg02099878 | | | N_Shore | NA | I | 2 |
| 250 | cg02100674 | NRG3; NRG3; NRG3 | Body; Body; TSS15 | S_Shore | NA | I | 10 |
| | | | 00 | | | | |
| 251 | cg02120658 | PAX3; PAX3; PAX3; P | Body; Body; Body; | Island | TRUE | I | 2 |
| | | AX3; PAX3; PAX3; PA | Body; Body Body; | | | | |
| | | X3; PAX3 | Body; Body | | | | |
| 252 | cg02134353 | | | Island | NA | II | 6 |
| 253 | cg02142896 | | | | TRUE | II | 14 |
| 254 | cg02144933 | AOX1 | TSS200 | Island | TRUE | II | 2 |
| 255 | cg02160684 | TRIM | Body | S_Shelf | NA | II | 8 |
| 256 | cg02167438 | PAK7; PAK7 | TSS200; TSS200 | Island | TRUE | I | 20 |
| 257 | cg02179029 | RBM41; RBM41 | Body; Body | N_Shelf | NA | II | X |
| 258 | cg02179455 | | | N_Shore | NA | II | 7 |
| 259 | cg02183671 | CD38; CD38 | 1stExon; 5′UTR | Island | NA | I | 4 |
| 260 | cg02192673 | NPFFR2 NPFFR2 | 1stExon; 5′UTR | Island | NA | II | 4 |
| 261 | cg02192855 | HIST1H2BI | TSS200 | N_Shore | NA | II | 6 |
| 262 | cg02197293 | FBXO27; FBXO27 | 5′UTR; 1stExon | Island | NA | I | 19 |
| 263 | cg02215070 | AKR1B1 | TSS200 | Island | NA | I | 7 |
| 264 | cg02231729 | HAPLN3 | 5′UTR | Island | NA | I | 15 |
| 265 | cg02233559 | SLC46A3; SLC46A3 | TSS200; TSS200 | Island | NA | II | 13 |
| 266 | cg02237629 | | | N_Shore | NA | I | 14 |
| 267 | cg02239640 | GSPT2 | 1stExon | S_Shore | NA | II | X |
| 268 | cg02242344 | | | N_Shore | NA | I | 2 |
| 269 | cg02245794 | TACC1; TACC1 | 1stExon; 5′UTR | | TRUE | II | 8 |
| 270 | cg02264082 | | | N_Shore | TRUE | I | 3 |
| 271 | cg02285155 | | | | TRUE | II | 10 |
| 272 | cg02288754 | SALL1; SALL1 | Body; 5′UTR | Island | NA | II | 16 |
| 273 | cg02305242 | FLRT2 | TSS1500 | N_Shore | NA | I | 14 |
| 274 | cg02305377 | | | N_Shelf | NA | I | 12 |
| 275 | cg02366534 | ME1 | TSS200 | Island | NA | II | 6 |
| 276 | cg02388453 | C15orf41; C15orf41 | Body; Body | | TRUE | I | 15 |
| 277 | cg02391441 | | | N_Shore | NA | II | 19 |
| 278 | cg02395819 | | | Island | TRUE | II | 12 |
| 279 | cg02415992 | GRIN2A; GRIN2A; GR | Body; Body; Body | | TRUE | II | 16 |
| | | IN2A | | | | | |
| 280 | cg02430935 | HMX2 | Body | Island | TRUE | I | 10 |
| 281 | cg02432280 | | | Island | NA | II | 7 |
| 282 | cg02433564 | | | Island | TRUE | I | 6 |
| 283 | cg02446647 | EYA4; EYA4; EYA4 | TSS1500; TSS1500 | Island | NA | I | 6 |
| | | | ; TSS1500 | | | | |
| 284 | cg02447304 | | | Island | NA | I | 6 |
| 285 | cg02454806 | LRFN5 | TSS1500 | N_Shore | NA | II | 14 |
| 286 | cg02457328 | | | S_Shelf | NA | II | 12 |
| 287 | cg02457623 | ATP2B4 ATP2B4 | 5′UTR; 5′UTR | S_Shore | NA | I | 1 |
| 288 | cg02460251 | | | Island | NA | I | 5 |
| 289 | cg02489958 | NKX6-1 | Body | N_Shore | NA | II | 4 |
| 290 | cg02490989 | MTMR7 | TSS1500 | S_Shore | NA | II | 8 |
| 291 | cg02493602 | ME3; ME3; ME3 | TSS1500; TSS200; | S_Shore | NA | I | 11 |
| | | | TSS1500 | | | | |
| 292 | cg02509086 | | | N_Shore | NA | II | 8 |
| 293 | cg02523640 | FOXI2 | TSS1500 | Island | NA | I | 10 |
| 294 | cg02532538 | | | Island | NA | I | 17 |
| 7 | cg02533058 | OPRM1; OPRM1; OPR | Body; Body; Body; | S_Shelf | NA | II | 6 |
| | | MI; OPRM1; OPRM1; | Body; Body; Body; | | | | |
| | | OPRM1; OPRM1; OPR | Body; Body; Body; 5 | | | | |
| | | MI; OPRM1; OPRM1; | ′UTR; Body; Body | | | | |
| | | OPRM1; OPRM1 | | | | | |
| 296 | cg02541778 | | | Island | NA | I | 8 |
| 297 | cg02553284 | COL2A1; COL2A1 | Body; Body | N_Shore | NA | I | 12 |
| 298 | cg02560085 | PCDHA6; PCDHA2; P | Body; Body; Body; T | N_Shore | NA | II | 5 |
| | | CDHA1; PCDHA8; PC | SS200; Body; Body; | | | | |
| | | DHA7; PCDHA1; PCD | Body; Body; TSS20 | | | | |
| | | HA6; PCDHA5; PCDH | 0; Body; Body | | | | |
| | | A8; PCDHA3; PCDHA | | | | | |
| | | 4 | | | | | |
| 299 | cg02564134 | ALDHLI | TSS1500 | S_Shore | TRUE | I | 3 |
| 300 | cg02578368 | HIST1H2BH; HIST1H | TSS1500; 1stExon | Island | NA | I | 6 |
| | | 3F | | | | | |
| 301 | cg02590060 | C1orf123 | Body | N_Shore | NA | II | 1 |
| 302 | cg02621287 | | | Island | NA | I | 12 |
| 303 | cg02628016 | KLHL24 | Body | | NA | II | 3 |
| 304 | cg02630214 | | | | NA | I | 3 |
| 305 | cg02637318 | ADAM32; ADAM32 | 1stExon; 5′UTR | Island | NA | I | 8 |
| 306 | cg02644510 | | | S_Shore | NA | I | 7 |
| 307 | cg02647878 | RNF175; RN175 | 5′UTR; 1stExon | Island | NA | I | 4 |
| 308 | cg02656891 | C1orf94; C1orf94 | 5′UTR; TSS200 | Island | NA | I | 1 |
| 309 | cg02707854 | SLC27A1 | Body | S_Shelf | NA | I | 19 |
| 310 | cg02708922 | ADI1 | Body | N_Shore | NA | I | 2 |
| 311 | cg02743222 | TLX3; TLX3 | 5′UTR; 1stExon | Island | NA | I | 5 |
| 312 | cg02757516 | GPR123 | Body | S_Shelf | NA | II | 10 |
| 313 | cg02764478 | SIM1 | Body | N_Shore | NA | II | 6 |
| 314 | cg02766845 | | | Island | NA | I | 6 |
| 315 | cg02796939 | FOXK2 | Body | N_Shore | NA | I | 17 |
| 316 | cg02809746 | | | Island | NA | I | 16 |
| 317 | cg02814558 | PRRG3 | Body | S_Shelf | NA | II | X |
| 318 | cg02818811 | GLIPR1L1 | TSS200 | | NA | I | 12 |
| 319 | cg02839824 | | | N_Shore | NA | II | X |
| 320 | cg02840535 | GTF3C1 | Body | | NA | I | 16 |
| 321 | cg02863073 | SYCN | 1stExon | Island | NA | I | 19 |
| 322 | cg02874376 | DLK1; DLK1 | 1stExon; 5′UTR | Island | NA | I | 14 |
| 323 | cg02879662 | HIF3A; HIF3A | Body; TSS1500 | Island | NA | I | 19 |
| 324 | cg02883150 | | | | NA | II | 2 |
| 325 | cg02883668 | KCNK10 | Body | N_Shore | TRUE | II | 14 |
| 326 | cg02888131 | ALDH1A2; ALDH1A2 | TSS200; TSS200 | Island | TRUE | II | 15 |
| 327 | cg02908900 | MEOX2 | TSS1500 | | TRUE | I | 7 |
| 328 | cg02909790 | HIST1H3G | 1stExon | Island | NA | II | 6 |
| 329 | cg02914422 | PDE1C | 5′UTR | Island | NA | I | 7 |
| 330 | cg02936263 | CCL7 | TSS1500 | | NA | II | 17 |
| 331 | cg02948624 | TBX1; TBX1; TBX1 | TSS1500; TSS1500 | Island | TRUE | II | 22 |
| | | | ; TSS1500 | | | | |
| 332 | cg02961934 | | | | NA | I | 7 |
| 333 | cg02988312 | KIAA1530 | Body | S_Shore | NA | I | 4 |
| 334 | cg03009240 | FZR1 | 5′UTR | S_Shore | NA | I | 19 |
| 335 | cg03012726 | | | N_Shelf | NA | I | 7 |
| 336 | cg03029734 | | | N_Shore | NA | II | 6 |
| 337 | cg03039990 | MIR770; MEG3; MEG3 | TSS1500; Body; Bo | | NA | II | 14 |
| | | ; MEG3 | dy; Body | | | | |
| 338 | cg03045635 | DRD5 | TSS200 | Island | NA | I | 4 |
| 339 | cg03048432 | NIN; NIN; NIN; NIN | 5′UTR; 5′UTR; TSS | | NA | II | 14 |
| | | | 1500; 5′UTR | | | | |
| 340 | cg03051777 | | | S_Shore | TRUE | II | 12 |
| 341 | cg03052128 | | | N_Shelf | NA | II | 12 |
| 342 | cg03094728 | AKAP9; AKAP9 | Body; Body | | TRUE | II | 7 |
| 343 | cg03097389 | TSC22D1; TSC22D1 | 3′UTR; 3′UTR | N_Shelf | TRUE | II | 13 |
| 344 | cg03110310 | ZNF205; ZNF205; MG | Body; Body; Body; | | NA | II | 16 |
| | | C3771; MGC3771 | Body | | | | |
| 345 | cg03131767 | ABCB9; ABCB9; ABC | 5′UTR; 5′UTR; 5′UT | S_Shore | NA | II | 12 |
| | | B9 | R | | | | |
| 346 | cg03142256 | | | S_Shore | NA | II | 3 |
| 347 | cg03158400 | FAM3B; FAM3B | Body; Body | Island | NA | I | 21 |
| 348 | cg03160883 | | | Island | NA | I | 6 |
| 349 | cg03161476 | ATP2B4; ATP2B4 | 5′UTR; 5′UTR | N_Shore | NA | II | 1 |
| 350 | cg03170013 | C2orf85 | TSS200 | N_Shelf | NA | II | 2 |
| 351 | cg03171770 | | | Island | NA | I | 10 |
| 352 | cg03174043 | | | N_Shore | NA | II | 2 |
| 353 | cg03175771 | PRR14 | TSS1500 | N_Shore | NA | I | 16 |
| 354 | cg03177042 | | | S_Shore | NA | I | 6 |
| 355 | cg03177600 | SLITRK3 | 5′UTR | | NA | II | 3 |
| 356 | cg03191045 | | | | NA | I | 20 |
| 357 | cg03195164 | C3orf37; C3orf37 | Body; Body | S_Shore | NA | II | 3 |
| 358 | cg03207574 | PRTFDC1 | Body | Island | NA | I | 10 |
| 359 | cg03209557 | HIST1H2BI; HIST1H2 | 3′UTR; 1stExon | S_Shore | NA | II | 6 |
| | | BI | | | | | |
| 360 | cg03212161 | KIRREL | Body | | TRUE | II | 1 |
| 361 | cg03217795 | PRKCB; PRKCB | 1stExon; 1stExon | Island | NA | I | 16 |
| 362 | cg03221483 | SEPT9; SEPT9; SEPT9 | 5′UTR; Body; TSS1 | | NA | II | 17 |
| | | | 500 | | | | |
| 363 | cg03227021 | | | Island | NA | I | 13 |
| 364 | cg03240800 | PCDHA6; PCDHA2; P | Body; Body; Body; | N_Shore | NA | II | 5 |
| | | CDHA1; PCDHA7; PC | Body; Body; TSS15 | | | | |
| | | DHA1; PCDHA8; PCD | 00; Body; Body; TSS | | | | |
| | | HA6; PCDHA5; PCDH | 1500; Body; Body | | | | |
| | | A8; PCDHA3; PCDHA | | | | | |
| | | 4 | | | | | |
| 365 | cg03241244 | KDM2B; KDM2B | Body; Body | Island | NA | I | 12 |
| 366 | cg03242698 | FLJ41350 | Body | S_Shore | NA | I | 10 |
| 367 | cg03257172 | NR5A2; NR5A2 | Body; Body | N_Shore | NA | I | 1 |
| 368 | cg03276000 | DPP6; DPP6; DPP6 | Body; Body; Body | | NA | II | 7 |
| 369 | cg03278146 | LOC642597 | TSS200 | Island | NA | I | 18 |
| 370 | cg03284392 | | | N_Shore | NA | II | 5 |
| 371 | cg03290400 | SIM1 | Body | N_Shore | TRUE | II | 6 |
| 372 | cg03296575 | | | S_Shelf | NA | II | 16 |
| 373 | cg03307401 | KLK13 | Body | Island | NA | I | 19 |
| 374 | cg03319638 | PLA2G4D | TSS1500 | | NA | II | 15 |
| 375 | cg03321020 | | | | NA | I | 1 |
| 376 | cg03330490 | HHEX | Body | Island | NA | I | 10 |
| 377 | cg03330678 | SEPT9; SEPT9; SEPT9; | 5′UTR; 5′UTR; Bod | | TRUE | II | 17 |
| | | SEPT9 | y; 1stExon | | | | |
| 378 | cg03336402 | EXOC4 | Body | | TRUE | II | 7 |
| 379 | cg03337918 | PLEKHF2 | 5′UTR | S_Shore | NA | II | 8 |
| 380 | cg03347018 | MSC | TSS200 | S_Shore | NA | I | 8 |
| 381 | cg03352106 | KCNJ8 | Body | | NA | I | 12 |
| 382 | cg03361164 | | | Island | NA | II | 6 |
| 383 | cg03362215 | | | S_Shelf | NA | II | 6 |
| 384 | cg03367264 | | | | NA | I | 10 |
| 385 | cg03388193 | HPSE2; HPSE2; HPSE2 | TSS1500; TSS1500 | S_Shore | NA | II | 10 |
| | | ; HPSE2 | ; TSS1500; TSS150 | | | | |
| | | | 0 | | | | |
| 386 | cg03397307 | EFCAB4B; EFCAB4B; | TSS200; TSS200; T | Island | TRUE | I | 12 |
| | | EFCAB4B | SS200 | | | | |
| 387 | cg03401357 | | | Island | NA | I | 1 |
| 388 | cg03420242 | | | Island | NA | II | 16 |
| 389 | cg03423123 | LOC644538 | 1stExon | Island | TRUE | II | X |
| 390 | cg03440272 | | | Island | TRUE | I | X |
| 391 | cg03454250 | | | | TRUE | II | 16 |
| 392 | cg03463076 | | | | TRUE | II | 11 |
| 393 | cg03464366 | | | | NA | II | 2 |
| 394 | cg03464514 | PTN; PTN | 5′UTR; 1stExon | | NA | II | 7 |
| 395 | cg03465499 | MIRGPRF; MRGPRF | TSS1500; TSS1500 | S_Shelf | NA | II | 11 |
| 396 | cg03465513 | | | Island | NA | I | 13 |
| 397 | cg03469862 | | | Island | NA | I | 11 |
| 398 | cg03478497 | C18orf34; C18orf34 | TSS1500; TSS1500 | S_Shore | NA | II | 18 |
| 399 | cg03479715 | FLJ43390 | Body | S_Shore | NA | II | 14 |
| 400 | cg03480698 | PTPRN2; PTPRN2; PT | Body; Body; Body | S_Shelf | NA | I | 7 |
| | | PRN2 | | | | | |
| 401 | cg03506640 | FLRT2 | 5′UTR | Island | NA | I | 14 |
| 402 | cg03512414 | RIN2 | Body | | TRUE | II | 20 |
| 403 | cg03520644 | COL11A1; COL11A1; | TSS1500; TSS1500 | | TRUE | I | 1 |
| | | COL11A1; COL11A1 | ; TSS1500; TSS150 | | | | |
| | | | 0 | | | | |
| 404 | cg03520938 | FAMI60B2 | 3′UTR | | NA | I | 8 |
| 405 | cg03522150 | C22orf49; C22orf49; C1 | Body; Body; Body; | | TRUE | II | 11 |
| | | 1orf49; C11orf49 | Body | | | | |
| 406 | cg03533858 | MORN1; RER1 | TSS1500; 5′UTR | S_Shore | NA | II | 1 |
| 407 | cg03539016 | | | | NA | I | 1 |
| 408 | cg03554749 | | | Island | NA | II | 4 |
| 409 | cg03558222 | | | Island | TRUE | II | X |
| 410 | cg03568017 | SEPT9; SEPT9; SEPT9 | 5′UTR; TSS200; Bo | | TRUE | II | 17 |
| | | | dy | | | | |
| 411 | cg03572772 | PCDHB15 | TSS1500 | N_Shore | NA | I | 5 |
| 412 | cg03583111 | DES | 1stExon | Island | NA | II | 2 |
| 414 | cg03598731 | | | Island | TRUE | I | 8 |
| 415 | cg03602500 | KIR3DX1 | Body | | NA | II | 19 |
| 416 | cg03616377 | | | Island | TRUE | II | 14 |
| 417 | cg03660500 | LHX9; LHX9 | Body; Body | Island | NA | II | 1 |
| 418 | cg03668718 | SLFN11; SLFN11; SLF | TSS1500; TSS1500 | Island | TRUE | I | 17 |
| | | N11; SLFN11; SLFN11 | ; TSS1500; TSS150 | | | | |
| | | | 0; TSS1500 | | | | |
| 419 | cg03671802 | RAX | TSS1500 | Island | NA | II | 18 |
| 420 | cg03679755 | | | Island | NA | I | 10 |
| 421 | cg03683587 | SHROOM4; SHROOM | TSS200; TSS200 | Island | NA | I | X |
| | | 4 | | | | | |
| 422 | cg03693842 | GABRA3 | TSS1500 | | NA | II | X |
| 423 | cg03704673 | GLTPD2; GLTPD2 | 5′UTR; 1stExon | Island | NA | I | 17 |
| 424 | cg03707308 | NRN1 | Body | Island | NA | I | 6 |
| 425 | cg03731268 | LITD1; LITD1 | 5′UTR; 5′UTR | Island | NA | I | 1 |
| 426 | cg03738384 | COTL1 | Body | | NA | I | 16 |
| 427 | cg03752218 | TAL1 | TSS1500 | Island | TRUE | I | 1 |
| 428 | cg03758514 | HIST1H2BI | TSS200 | N_Shore | NA | II | 6 |
| 429 | cg03780486 | OR6A2 | TSS200 | | NA | II | 11 |
| 430 | cg03784083 | OPCML; OPCML | Body; Body | | TRUE | I | 11 |
| 431 | cg03787864 | CYBA | Body | Island | NA | I | 16 |
| 432 | cg03790988 | DOK6 | Body | Island | NA | I | 18 |
| 433 | cg03804500 | ANAPC2; SSNA1 | Body; TSS1500 | N_Shore | NA | II | 9 |
| 434 | cg03815116 | YIPF7 | TSS1500 | | NA | II | 4 |
| 435 | cg03830329 | SIM1 | Body | Island | NA | II | 6 |
| 436 | cg03857553 | NIPSNAP1 | Body | N_Shore | NA | II | 22 |
| 437 | cg03867465 | MGC45800 | Body | Island | NA | I | 4 |
| 438 | cg03880173 | SOX2OT | Body | Island | TRUE | II | 3 |
| 439 | cg03887163 | LMX1A | Body | N_Shore | NA | II | 1 |
| 440 | cg03889013 | | | N_Shore | NA | II | 10 |
| 441 | cg03890037 | LVRN | TSS200 | Island | TRUE | II | 5 |
| 442 | cg03892838 | LRRFIP1; LRRFIP1; L | TSS1500; TSS1500 | N_Shore | NA | I | 2 |
| | | RRF1P1; LRRFIP1; LR | ; TSS1500; TSS150 | | | | |
| | | RFIP1 | 0; Body | | | | |
| 443 | cg03895540 | OR52N2 | TSS200 | | NA | II | 11 |
| 444 | cg03900143 | ZIC4; ZIC4; ZIC4; ZIC4 | Body; TSS1500; Bo | Island | NA | I | 3 |
| | | ; ZIC4 | dy; Body; TSS1500 | | | | |
| 445 | cg03916909 | | | Island | NA | I | 20 |
| 446 | cg03950785 | | | N_Shore | NA | II | X |
| 447 | cg03951374 | MEIS2; MEIS2; MEIS2 | 1stExon; Body; Bod | Island | NA | II | 15 |
| | | ; MEIS2; MEIS2; MEIS | y; 5′UTR; Body; Bod | | | | |
| | | 2; MEIS2 | y; Body | | | | |
| 448 | cg03953660 | | | Island | TRUE | I | 2 |
| 449 | cg03962217 | LOC202781 | Body | S_Shore | NA | II | 7 |
| 450 | cg03966514 | SPG20; SPG20; SPG20; | 5′UTR; 5′UTR; 5′UT | Island | NA | I | 13 |
| | | SPG20 | R; 5′UTR | | | | |
| 451 | cg03981954 | FEZF1; FEZF1 | 3′UTR; 3′UTR | N_Shore | NA | I | 7 |
| 452 | cg03985727 | MEOX2 | TSS1500 | | TRUE | II | 7 |
| 453 | cg03989125 | | | Island | TRUE | I | 22 |
| 454 | cg03999216 | | | Island | NA | II | 2 |
| 455 | cg04003327 | ESPNL; SCLY | TSS1500; 3′UTR | N_Shore | TRUE | II | 2 |
| 456 | cg04003850 | FGF12; FGF12; FGF12 | 5′UTR; 1stExon; Bo | Island | NA | II | 3 |
| | | | dy | | | | |
| 457 | cg04007726 | ZMIZ1 | Body | | NA | I | 10 |
| 458 | cg04011247 | BCAT1; BCAT1 | 1stExon; 5′UTR | 5 Shore | NA | I | 12 |
| 459 | cg04025964 | NR5A2; NR5A2 | Body; Body | Island | NA | I | 1 |
| 460 | cg04041976 | TRIM55; TRIM55; TRI | Body; Body; Body; | N_Shelf | NA | I | 8 |
| | | M55; TRIM55 | Body | | | | |
| 461 | cg04052466 | AMH | Body | Island | NA | I | 19 |
| 462 | cg04058937 | | | Island | NA | I | 15 |
| 463 | cg04066019 | RFTN1 | 5′UTR | Island | NA | I | 3 |
| 464 | cg04093225 | ILDR2 | Body | Island | NA | I | 1 |
| 465 | cg04098866 | | | Island | NA | I | 16 |
| 466 | cg04101806 | AFF3; AFF3 | TSS1500; 5′UTR | S_Shore | TRUE | II | 2 |
| 467 | cg04108939 | BEST4 | Body | Island | TRUE | I | 1 |
| 468 | cg04111071 | TMEM196 | TSS1500 | S_Shore | NA | I | 7 |
| 469 | cg04120272 | FBXO47 | TSS200 | | NA | II | 17 |
| 470 | cg04126266 | PAX7; PAX7; PAX7 | Body; Body; Body | N_Shore | NA | II | 1 |
| 471 | cg04131898 | WIT1 | Body | Island | TRUE | I | 11 |
| 472 | cg04150495 | MIR663 | TSS1500 | Island | NA | I | 20 |
| 473 | cg04151012 | ZNF512 | Body | S_Shore | NA | II | 2 |
| 474 | cg04151062 | | | Island | TRUE | II | 1 |
| 475 | cg04153740 | | | Island | TRUE | II | 16 |
| 476 | cg04156293 | ACHE; ACHE | Body; Body | N_Shore | NA | I | 7 |
| 477 | cg04164058 | | | Island | NA | II | 15 |
| 478 | cg04174091 | | | 5 Shore | NA | II | 11 |
| 479 | cg04177426 | | | Island | NA | I | X |
| 480 | cg04184019 | | | S_Shore | NA | I | 4 |
| 481 | cg04194494 | ALOX5 | Body | Island | NA | I | 10 |
| 482 | cg04195774 | TNFAIP8 | Body | | TRUE | II | 5 |
| 483 | cg04206484 | C7orf50; C7orf50; C7or | Body; Body; Body | S_Shore | TRUE | I | 7 |
| | | f50 | | | | | |
| 484 | cg04215800 | | | Island | TRUE | II | 14 |
| 485 | cg04221650 | LCE4A; LCE4A | 1stExon; 3′UTR | | NA | II | 1 |
| 486 | cg04227631 | GABRA3; MIR767; | 5′UTR; TSS1500; T | | NA | II | X |
| | | M1R105-1 | SS1500 | | | | |
| 487 | cg04244354 | | | N_Shore | TRUE | II | 8 |
| 488 | cg04248271 | EBF1 | Body | Island | TRUE | I | 5 |
| 489 | cg04281464 | | | Island | NA | I | 14 |
| 490 | cg04284140 | POLD2; POLD2 | TSS1500; TSS1500 | S_Shore | NA | II | 7 |
| 491 | cg04285033 | | | Island | TRUE | I | 21 |
| 492 | cg04294888 | TTC28 | Body | | NA | II | 22 |
| 493 | cg04299439 | | | N_Shore | NA | II | 2 |
| 494 | cg04302300 | | | Island | NA | I | X |
| 495 | cg04313756 | TCL1A; TCL1A | TSS1500; TSS1500 | Island | NA | I | 14 |
| 496 | cg04319276 | | | Island | NA | II | 19 |
| 497 | cg04330513 | SFTA3 | TSS1500 | Island | TRUE | I | 14 |
| 498 | cg04337056 | GFM1 | TSS1500 | N_Shore | NA | II | 3 |
| 499 | cg04343891 | PRDM8; PRDM8 | 5′UTR; 1stExon | S_Shore | NA | I | 4 |
| 500 | cg04349243 | SLC16A12 | 5′UTR | Island | TRUE | I | 10 |
| 501 | cg04365202 | LHX9; LHX9 | Body; Body | N_Shore | NA | II | 1 |
| 502 | cg04380340 | AOX1 | TSS1500 | N_Shore | TRUE | II | 2 |
| 503 | cg04388901 | MAP4; MAP4; MAP4 | 5′UTR; 5′UTR; 5′UT | | TRUE | II | 3 |
| 504 | cg04389709 | | | Island | NA | II | 12 |
| 505 | cg04399994 | PAK3; PAK3; PAK3 | 5′UTR; 5′UTR; TSS | N_Shore | NA | II | X |
| | | | 1500 | | | | |
| 506 | cg04401986 | FGF5; FGF5 | Body; Body | S_Shelf | NA | I | 4 |
| 507 | cg04406436 | | | Island | NA | I | 1 |
| 508 | cg04407470 | NR2E1 | Body | Island | TRUE | I | 6 |
| 509 | cg04421553 | VILL | Body | S_Shore | NA | II | 3 |
| 510 | cg04430582 | MIR195; MIR497 | TSS1500; TSS1500 | N_Shelf | NA | II | 17 |
| 511 | cg04452095 | SEPT9; SEPT9; SEPT9; | 5′UTR; 5′UTR; Bod | | TRUE | II | 17 |
| | | SEPT9 | y; 1stExon | | | | |
| 512 | cg04460984 | CHRNB4 | TSS200 | Island | TRUE | I | 15 |
| 513 | cg04461197 | SPATA16 | Body | | TRUE | II | 3 |
| 514 | cg04467162 | | | Island | NA | I | 3 |
| 515 | cg04467618 | TCF21; TCF21 | 1stExon; 1stExon | Island | NA | II | 6 |
| 516 | cg04479219 | SLITRK5 | 5′UTR | Island | NA | II | 13 |
| 517 | cg04498418 | GRIK2; GRIK2; GRIK2 | Body; Body; Body | Island | TRUE | II | 6 |
| 518 | cg04528545 | | | S_Shore | NA | II | X |
| 519 | cg04549333 | ALX4 | Body | Island | TRUE | I | 11 |
| 520 | cg04550052 | SALL1; SALL1; SALL | Body; 5′UTR; 1stEx | Island | NA | I | 16 |
| | | 1 | on | | | | |
| 521 | cg04554272 | LMF1 | Body | N_Shore | NA | I | 16 |
| 522 | cg04560456 | LIN28B; LIN28B | 1stExon; 5′UTR | S_Shelf | NA | II | 6 |
| 523 | cg04560832 | MRPS21; MRPS21 | Body; Body | S_Shore | NA | II | 1 |
| 524 | cg04562217 | ROBO1 | 5′UTR | Island | NA | I | 3 |
| 525 | cg04567738 | | | N_Shore | TRUE | II | 1 |
| 526 | cg04574507 | CD1B | Body | | NA | II | 1 |
| 527 | cg04578774 | ALX4 | TSS1500 | Island | NA | I | 11 |
| 528 | cg04612444 | PENK; PENK | 5′UTR; TSS200 | Island | NA | I | 8 |
| 529 | cg04622802 | FIBIN; FIBIN | 5′UTR; 1stExon | | TRUE | II | 11 |
| 530 | cg04628320 | TBCB | TSS1500 | Island | NA | II | 19 |
| 531 | cg04634417 | PCDHA6; PCDHA2; P | Body; Body; Body; T | N_Shore | NA | II | 5 |
| | | CDHA1; PCDHA8; PC | SS200; Body; Body; | | | | |
| | | DHA7; PCDHA1; PCD | Body; Body; TSS20 | | | | |
| | | HA6; PCDHA5; PCDH | 0; Body; Body | | | | |
| | | A8; PCDHA3; PCDHA | | | | | |
| | | 4 | | | | | |
| 532 | cg04638468 | TMEM155; LOC10019 | TSS200; Body | Island | NA | I | 4 |
| | | 2379 | | | | | |
| 533 | cg04640675 | | | Island | NA | I | 16 |
| 534 | cg04650676 | SEPT9; SEPT9; SEPT9 | 5′UTR; TSS200; Bo | | TRUE | I | 17 |
| | | | dy | | | | |
| 535 | cg04652097 | MAGI2 | TSS1500 | Island | NA | I | 7 |
| 536 | cg04653522 | RAB38 | TSS200 | S_Shore | NA | II | 11 |
| 537 | cg04656042 | | | Island | TRUE | I | 19 |
| 538 | cg04660410 | VILL | Body | S_Shore | NA | II | 3 |
| 539 | cg04670802 | | | N_Shore | NA | I | 6 |
| 541 | cg04688351 | PAX3; PAX3; PAX3; P | Body; Body; Body; | N_Shore | NA | I | 2 |
| | | AX3; PAX3; PAX3 | Body; Body; Body | | | | |
| 542 | cg04692403 | TCF21; TCF21 | 1stExon; 1stExon | Island | NA | II | 6 |
| 543 | cg04698114 | SALL1; SALL1; SALL | Body; 5′UTR; 1stEx | Island | NA | I | 16 |
| | | 1 | on | | | | |
| 544 | cg04701034 | NID2 | TSS1500 | Island | NA | I | 14 |
| 545 | cg04722177 | | | Island | TRUE | I | 19 |
| 546 | cg04723401 | | | | NA | I | 14 |
| 547 | cg04728482 | | | Island | NA | I | 5 |
| 548 | cg04738552 | TUBB4 | TSS1500 | S_Shore | NA | I | 19 |
| 549 | cg04738965 | ZIC1; ZIC1 | 1stExon; 5′UTR | Island | TRUE | I | 3 |
| 550 | cg04761722 | GRIN2A; GRIN2A; GR | 5′UTR; 5′UTR; 5′UT | Island | NA | I | 16 |
| | | IN2A | R | | | | |
| 551 | cg04771206 | POLR1A; PTCD3 | Body; TSS1500 | N_Shore | NA | II | 2 |
| 552 | cg04772683 | KLK10; KLK10; KLK1 | 5′UTR; 5′UTR; 5′UT | Island | NA | I | 19 |
| | | 0 | R | | | | |
| 553 | cg04804377 | RAD51AP2 | TSS200 | | NA | I | 2 |
| 554 | cg04806942 | | | Island | NA | II | 14 |
| 555 | cg04828133 | | | Island | NA | I | 7 |
| 556 | cg04832767 | DLX6AS | Body | N_Shore | NA | I | 7 |
| 557 | cg04846243 | IGF2BP1; IGF2BP1; IG | 1stExon; 1stExon; 5′ | Island | NA | I | 17 |
| | | F2BP1; IGF2BP1 | UTR; 5′UTR | | | | |
| 558 | cg04850731 | NXPH4 | Body | Island | NA | I | 12 |
| 559 | cg04851268 | GHSR; GHSR | TSS1500; TSS1500 | N_Shore | TRUE | II | 3 |
| 560 | cg04859726 | SIM1 | Body | N_Shore | NA | II | 6 |
| 561 | cg04868078 | LHX9; LHX9 | Body; Body | | NA | I | 1 |
| 562 | cg04902729 | CACNA1E | TSS200 | Island | NA | II | 1 |
| 563 | cg04904331 | VWC2 | TSS1500 | Island | NA | I | 7 |
| 564 | cg04910834 | | | | TRUE | II | 8 |
| 565 | cg04911050 | RAB40C | 3′UTR | Island | NA | I | 16 |
| 566 | cg04927889 | FEZF2 | TSS1500 | S_Shore | NA | I | 3 |
| 567 | cg04931126 | | | | TRUE | II | 3 |
| 568 | cg04932340 | | | Island | NA | I | 14 |
| 569 | cg04935434 | | | Island | NA | I | 2 |
| 570 | cg04937184 | BMP4; BMP4 | TSS1500; TSS1500 | S_Shore | NA | I | 14 |
| 571 | cg04954680 | LRRC3B | TSS1500 | N_Shore | NA | II | 3 |
| 572 | cg04958794 | | | Island | NA | II | 14 |
| 573 | cg04961042 | PRKD1 | Body | | TRUE | II | 14 |
| 574 | cg04982193 | C8orf33 | Body | S_Shore | NA | II | 8 |
| 575 | cg05016408 | LOC134466 | TSS200 | Island | NA | I | 5 |
| 576 | cg05019826 | | | | TRUE | I | 15 |
| 577 | cg05024627 | | | | NA | II | 7 |
| 578 | cg05036656 | | | Island | TRUE | II | 4 |
| 579 | cg05042492 | | | N_Shelf | NA | II | 15 |
| 580 | cg05049335 | RIN1; RIN1 | 1stExon; 5′UTR | S_Shore | TRUE | II | 11 |
| 581 | cg05059304 | SYCP2L | TSS200 | N_Shore | NA | I | 6 |
| 582 | cg05060976 | CABYR; CABYR; CA | 3′UTR; 3′UTR; 3′UT | | NA | II | 18 |
| | | BYR; CABYR; CABY | R; 3′UTR; 3′UTR; 3 | | | | |
| | | R; CABYR | UTR | | | | |
| 583 | cg05079544 | N4BP1 | TSS1500 | S_Shore | NA | II | 16 |
| 584 | cg05085500 | | | Island | NA | II | 6 |
| 585 | cg05090759 | SLC38A11 | TSS200 | | NA | I | 2 |
| 586 | cg05099464 | ARHGEF12 | Body | | TRUE | II | 11 |
| 587 | cg05100070 | SIM1 | Body | N_Shore | NA | II | 6 |
| 588 | cg05116382 | ACTN3 | Body | S_Shore | NA | I | 11 |
| 589 | cg05119057 | C14orf23 | Body | S_Shore | NA | I | 14 |
| 590 | cg05128414 | LHX9 | Body | S_Shore | NA | II | 1 |
| 591 | cg05133179 | | | Island | TRUE | I | 11 |
| 592 | cg05134041 | POU3F4 | TSS200 | N_Shore | NA | I | X |
| 593 | cg05135644 | NR2EI | Body | Island | TRUE | II | 6 |
| 594 | cg05137386 | | | N_Shore | NA | I | 14 |
| 595 | cg05143887 | ACSS3 | 1stExon | Island | NA | I | 12 |
| 596 | cg05156137 | RCAN1; RCAN1; RCA | 5′UTR; Body; 1stEx | | TRUE | II | 21 |
| | | N1 | on | | | | |
| 597 | cg05159188 | HIST1H4F | TSS200 | N_Shore | NA | II | 6 |
| 598 | cg05161795 | ZDHHC3; ZDHHC3; E | TSS1500; TSS1500 | S_Shore | NA | II | 3 |
| | | XOSC7; ZDHHC3; EX | ; Body; TSS1500; Bo | | | | |
| | | OSC7 | dy | | | | |
| 599 | cg05162032 | CYTH3 | Body | N_Shore | NA | II | 7 |
| 600 | cg05174942 | VTRNA1-2 | TSS200 | | NA | II | 5 |
| 601 | cg05182265 | UBE3C | Body | S_Shore | TRUE | II | 7 |
| 602 | cg05200373 | SLC39A7; SLC39A7 | Body; Body | S_Shore | NA | II | 6 |
| 603 | cg05210558 | NTM | Body | | NA | II | 11 |
| 604 | cg05211057 | COL9A1; COL9A1 | Body; Body | Island | TRUE | II | 6 |
| 605 | cg05224998 | SLFN11; SLFN11; SLF | TSS200; TSS200; T | N_Shore | TRUE | I | 17 |
| | | N11; SLFN11; SLFN11 | SS200; TSS200; TS | | | | |
| | | | S200 | | | | |
| 606 | cg05239680 | | | | NA | I | X |
| 607 | cg05241828 | GPT | TSS1500 | N_Shore | NA | I | 8 |
| 608 | cg05247035 | | | | TRUE | II | 8 |
| 609 | cg05257528 | PRTN3 | Body | Island | NA | II | 19 |
| 610 | cg05270344 | MAGI2 | TSS1500 | Island | NA | II | 7 |
| 611 | cg05275752 | GALM | TSS200 | | NA | II | 2 |
| 612 | cg05280814 | | | N_Shelf | NA | II | 11 |
| 613 | cg05285429 | ROBO2 | Body | | TRUE | II | 3 |
| 614 | cg05291374 | DIXDC1; DIXDC1 | TSS1500; Body | N_Shore | NA | II | 11 |
| 615 | cg05298458 | TBX1; TBX1; TBX1 | Body; Body; Body | Island | NA | II | 22 |
| 616 | cg05298922 | | | Island | NA | II | 2 |
| 617 | cg05304531 | DHDDS; HMGN2; DH | 3′UTR; TSS1500; 3′ | N_Shore | TRUE | I | 1 |
| | | DDS | UTR | | | | |
| 618 | cg05308504 | | | Island | NA | I | 16 |
| 619 | cg05314639 | | | | NA | II | 13 |
| 620 | cg05327864 | CHRM2; CHRM2; CH | 5′UTR; 5′UTR; 5′UT | Island | TRUE | II | 7 |
| | | RM2; CHRM2; CHRM | R; 5′UTR; 5′UTR; 5′ | | | | |
| | | 2; CHRM2; CHRM2; C | UTR; 5′UTR; 5′UTR | | | | |
| | | HIRM2 | | | | | |
| 621 | cg05339695 | FLJ43390 | Body | S_Shore | NA | II | 14 |
| 622 | cg05345154 | AMH | Body | Island | NA | I | 19 |
| 623 | cg05357932 | ZNRF2 | Body | S_Shore | NA | II | 7 |
| 624 | cg05372242 | SFTA3 | TSS1500 | Island | TRUE | I | 14 |
| 625 | cg05373457 | KCNS2 | 5′UTR | Island | NA | II | 8 |
| 626 | cg05376362 | | | | TRUE | II | 6 |
| 627 | cg05387146 | ME1 | TSS200 | Island | NA | I | 6 |
| 628 | cg05391318 | NR5A2; NR5A2 | Body; Body | Island | NA | I | 1 |
| 629 | cg05393297 | | | N_Shore | TRUE | I | 12 |
| 630 | cg05400200 | MLL5; MLL5 | Body; Body | | NA | II | 7 |
| 631 | cg05414338 | | | N_Shore | NA | II | 6 |
| 632 | cg05446860 | LOC284798; LOC2847 | Body; TSS1500; Bo | Island | TRUE | II | 20 |
| | | 98; LOC284798 | dy | | | | |
| 633 | cg05471509 | | | | NA | II | 13 |
| 634 | cg05482942 | | | Island | NA | I | 10 |
| 635 | cg05493394 | EYA4; EYA4; EYA4 | TSS1500; TSS1500 | N_Shore | NA | II | 6 |
| | | | ; TSS1500 | | | | |
| 636 | cg05494014 | MEOX2 | 1stExon | | NA | I | 7 |
| 637 | cg05496203 | MEIS1 | Body | S_Shelf | NA | II | 2 |
| 638 | cg05500015 | NAALAD2; NAALAD | 5′UTR; 1stExon | | NA | II | 11 |
| | | 2 | | | | | |
| 639 | cg05507490 | | | Island | NA | I | 2 |
| 640 | cg05525812 | | | N_Shore | NA | I | 2 |
| 641 | cg05557266 | DALRD3; DALRD3; N | 5′UTR; TSS1500; T | N_Shore | NA | II | 3 |
| | | DUFAF3; NDUFAF3 | SS1500; TSS1500 | | | | |
| 642 | cg05560435 | | | Island | TRUE | I | 5 |
| 643 | cg05562381 | | | Island | TRUE | I | 17 |
| 644 | cg05576949 | IGF2BP2; IGF2BP 2 | Body; Body | N_Shore | NA | II | 3 |
| 645 | cg05583608 | | | Island | TRUE | I | 16 |
| 646 | cg05626117 | CLEC4G | TSS1500 | S_Shore | NA | II | 19 |
| 647 | cg05648914 | | | | TRUE | II | 5 |
| 648 | cg05658512 | C7orf27 | 5′UTR | N_Shore | NA | II | 7 |
| 649 | cg05663573 | SLC7A14 | 5′UTR | N_Shore | NA | I | 3 |
| 650 | cg05676622 | RBM4 | Body | Island | NA | I | 11 |
| 651 | cg05680973 | HS6ST2; HS6ST2 | Body; Body | N_Shore | NA | II | X |
| 652 | cg05696715 | ABT1 | Body | Island | NA | II | 6 |
| 653 | cg05733181 | | | Island | TRUE | II | 4 |
| 654 | cg05735180 | | | Island | NA | I | 18 |
| 655 | cg05736424 | | | S_Shelf | NA | II | 2 |
| 656 | cg05736768 | DPYS; DPYS | 1stExon; 5′UTR | Island | NA | I | 8 |
| 657 | cg05743346 | CALN1; CALN1 | 5′UTR; Body | Island | NA | II | 7 |
| 658 | cg05755900 | PFDN6; WDR46; WDR | Body; TSS1500; TS | S_Shore | NA | I | 6 |
| | | 46 | S1500 | | | | |
| 659 | cg05768044 | CHD9 | Body | | NA | II | 16 |
| 660 | cg05769889 | DUSP6; DUSP6 | Body; Body | N_Shore | NA | I | 12 |
| 661 | cg05774229 | | | Island | TRUE | I | 11 |
| 662 | cg05783185 | EXT1 | Body | N_Shelf | NA | II | 8 |
| 663 | cg05790451 | APBA2; APBA2 | Body; Body | Island | TRUE | I | 15 |
| 664 | cg05793120 | | | Island | NA | I | 4 |
| 665 | cg05803296 | ATXN7L1; ATXN7L1 | TSS200; Body | | TRUE | II | 7 |
| 666 | cg05852143 | NTF3 | TSS1500 | Island | NA | I | 12 |
| 667 | cg05854114 | | | Island | NA | I | 12 |
| 668 | cg05861639 | KLHL28 | 5′UTR | N_Shelf | NA | II | 14 |
| 669 | cg05894124 | FGF5; FGF5 | TSS1500; TSS1500 | N_Shore | TRUE | II | 4 |
| 670 | cg05901543 | CDH15 | Body | | NA | I | 16 |
| 671 | cg05905176 | TNFAIP8L3 | Body | Island | NA | I | 15 |
| 672 | cg05907046 | ZSWIM2 | TSS200 | Island | TRUE | II | 2 |
| 673 | cg05916684 | | | N_Shore | NA | I | 17 |
| 674 | cg05918002 | ZNF232 | 5′UTR | Island | NA | I | 17 |
| 675 | cg05920909 | LHX9; LHX9 | Body; Body | N_Shore | TRUE | II | 1 |
| 676 | cg05923687 | | | Island | TRUE | I | 1 |
| 677 | cg05937630 | AKR7A3 | TSS1500 | Island | NA | I | 1 |
| 678 | cg05937737 | SLC7A14 | 5′UTR | Island | NA | I | 3 |
| 679 | cg05940231 | TBXI5 | TSS200 | Island | TRUE | I | 1 |
| 680 | cg05940452 | FOXRI; FOXR1 | 5′UTR; 1stExon | Island | NA | I | 11 |
| 681 | cg05944877 | PRKCB; PRKCB | Body; Body | | TRUE | II | 16 |
| 682 | cg05945266 | FOXK2 | Body | N_Shore | NA | II | 17 |
| 683 | cg05982460 | | | S_Shelf | NA | I | 16 |
| 684 | cg05991442 | RECQL5; LOC643008; | Body; Body; 5′UTR | | NA | I | 17 |
| | | LOC643008 | | | | | |
| 685 | cg05994094 | C18orf34; C18orf34 | TSS200; TSS1500 | Island | NA | I | 18 |
| 686 | cg05994320 | | | Island | NA | I | 7 |
| 687 | cg06008926 | | | N_Shore | NA | I | 5 |
| 688 | cg06014401 | EOMES | TSS1500 | Island | NA | II | 3 |
| 689 | cg06022561 | D2HGDH | Body | | NA | I | 2 |
| 690 | cg06022664 | MIR548H4 | Body | | NA | I | 15 |
| 691 | cg06024295 | CPN1; CPN1 | 1stExon; 5′UTR | | NA | II | 10 |
| 692 | cg06025456 | PPT2; PRRT1; PPT2 | TSS1500; TSS1500 | N_Shore | TRUE | I | 6 |
| | | | ; TSS1500 | | | | |
| 693 | cg06038944 | | | | TRUE | II | 2 |
| 694 | cg06057569 | BRD1 | TSS1500 | N_Shore | NA | II | 22 |
| 695 | cg06077978 | PCSK1N | Body | Island | NA | I | X |
| 696 | cg06083330 | POU4F2 | Body | Island | TRUE | I | 4 |
| 697 | cg06087421 | TAL1; TAL1 | 5′UTR; 1stExon | N_Shore | NA | I | 1 |
| 698 | cg06092815 | SPHKAP; SPHKAP | TSS200; TSS200 | Island | NA | II | 2 |
| 699 | cg06108782 | SLC18A3; CHAT; CHA | TSS1500; 1stExon; | Island | NA | II | 10 |
| | | T | 5′UTR | | | | |
| 700 | cg06120122 | | | | TRUE | II | 11 |
| 701 | cg06122635 | | | Island | NA | I | 2 |
| 702 | cg06132727 | | | Island | NA | I | 4 |
| 703 | cg06148685 | LPAR1; LPAR1 | TSS1500; TSS1500 | Island | NA | II | 9 |
| 704 | cg06179765 | GPNMB; GPNMB | Body; Body | Island | TRUE | I | 7 |
| 705 | cg06184669 | GABRB1 | Body | N_Shore | TRUE | II | 4 |
| 706 | cg06193578 | | | Island | TRUE | II | 19 |
| 707 | cg06211837 | ST6GALNAC3; ST6G | 1stExon; 5′UTR; 1st | Island | NA | I | 1 |
| | | ALNAC3; ST6GALNA | Exon; 5′UTR | | | | |
| | | C3; ST6GALNAC3 | | | | | |
| 708 | cg06212263 | | | S_Shore | NA | II | 21 |
| 709 | cg06217046 | WWC3 | Body | Island | NA | II | X |
| 710 | cg06221393 | | | Island | NA | II | 5 |
| 711 | cg06240450 | ALDH1L1 | TSS200 | Island | TRUE | I | 3 |
| 712 | cg06253058 | WWTR1; WWTR1; W | Body; Body; Body | Island | NA | II | 3 |
| | | WTR1 | | | | | |
| 713 | cg06254453 | | | N_Shore | NA | II | 6 |
| 714 | cg06264868 | | | | NA | II | 10 |
| 715 | cg06271630 | | | | NA | II | 10 |
| 716 | cg06271970 | | | Island | NA | I | 15 |
| 717 | cg06274159 | ZFP42 | TSS200 | Island | NA | II | 4 |
| 718 | cg06282524 | | | Island | NA | I | X |
| 719 | cg06284231 | CLEC14A | 1stExon | Island | NA | II | 14 |
| 720 | cg06286796 | PSMD6 | Body | N_Shore | NA | II | 3 |
| 721 | cg06288788 | | | S_Shore | NA | II | 16 |
| 722 | cg06296503 | | | S_Shore | NA | I | 6 |
| 723 | cg06304401 | NLGN1 | 5′UTR | | NA | I | 3 |
| 724 | cg06312254 | SLITRK3 | 5′UTR | | NA | II | 3 |
| 725 | cg06321998 | | | | NA | II | 6 |
| 726 | cg06330323 | TSC2; TSC2; TSC2 | Body; Body; Body | N_Shore | TRUE | I | 16 |
| 727 | cg06356912 | VILL | Body | N_Shore | NA | II | 3 |
| 728 | cg06369992 | | | Island | NA | II | 10 |
| 729 | cg06371502 | | | N_Shore | TRUE | I | 1 |
| 730 | cg06385187 | WFDC2 | TSS200 | Island | NA | I | 20 |
| 731 | cg06388730 | | | Island | NA | II | 7 |
| 732 | cg06401021 | HMGCLL1; HMGCLL | 1stExon; 5′UTR; 1st | Island | TRUE | I | 6 |
| | | 1; HMGCLL1; HMGCL | Exon; 5′UTR | | | | |
| | | L1 | | | | | |
| 733 | cg06404175 | OXT | Body | Island | TRUE | I | 20 |
| 734 | cg06410057 | | | S_Shore | NA | II | 8 |
| 735 | cg06411724 | MBOX2 | Body | | NA | I | 7 |
| 736 | cg06424065 | | | Island | NA | I | 4 |
| 737 | cg06428055 | ELF4; ELF4 | 5′UTR; 5′UTR | Island | NA | II | X |
| 738 | cg06439589 | SSR4 | Body | S_Shore | NA | II | X |
| 739 | cg06444282 | VWA5B1 | Body | Island | NA | I | 1 |
| 740 | cg06465306 | ZNF711 | Body | S_Shelf | NA | II | X |
| 741 | cg06467636 | CNGA2 | 5′UTR | | NA | I | X |
| 742 | cg06470822 | | | Island | TRUE | II | 6 |
| 743 | cg06482019 | | | Island | NA | I | 16 |
| 744 | cg06495961 | DPP6; DPP6 | 1stExon; 5′UTR | Island | NA | II | 7 |
| 745 | cg06503907 | SYCP2L | TSS200 | N_Shore | NA | I | 6 |
| 746 | cg06520675 | FLJ41350 | Body | Island | TRUE | II | 10 |
| 747 | cg06580523 | | | N_Shelf | NA | II | 12 |
| 748 | cg06583549 | IRF2BP1 | 1stExon | Island | TRUE | I | 19 |
| 749 | cg06587521 | LRIG3; LRIG3 | Body; Body | | TRUE | II | 12 |
| 750 | cg06598544 | COL9A3 | 3′UTR | Island | NA | I | 20 |
| 751 | cg06609094 | | | N_Shelf | TRUE | II | 4 |
| 752 | cg06610484 | MCHR2; MCHR2; MC | TSS200; 5′UTR; 1st | Island | TRUE | I | 6 |
| | | HR2 | Exon | | | | |
| 753 | cg06620896 | | | S_Shore | NA | I | 15 |
| 754 | cg06633061 | | | Island | NA | II | 14 |
| 755 | cg06645033 | | | Island | NA | I | 1 |
| 756 | cg06650546 | | | Island | NA | I | X |
| 757 | cg06654901 | PAX1 | Body | Island | NA | II | 20 |
| 758 | cg06660332 | | | Island | TRUE | I | 1 |
| 759 | cg06661450 | LHX9; LHX9 | Body; Body | N_Shore | NA | II | 1 |
| 760 | cg06666008 | PAX6; PAX6; PAX6 | Body; Body; Body | N_Shore | NA | I | 11 |
| 761 | cg06693048 | EYA1 | TSS1500 | | NA | II | 8 |
| 762 | cg06704518 | GATA4 | 5′UTR | Island | NA | II | 8 |
| 763 | cg06711380 | FBXO47 | TSS1500 | | NA | II | 17 |
| 764 | cg06717750 | RAB40C | Body | N_Shelf | NA | I | 16 |
| 765 | cg06720669 | KCNE1L; KCNE1L | 1stExon; 5′UTR | Island | TRUE | I | X |
| 766 | cg06729532 | | | Island | NA | I | 19 |
| 767 | cg06744545 | | | Island | TRUE | I | 2 |
| 768 | cg06747432 | CCDC8; CCDC8 | 1stExon; 5′UTR | Island | NA | I | 19 |
| 769 | cg06755825 | | | Island | NA | II | 15 |
| 770 | cg06759629 | | | Island | TRUE | II | 4 |
| 771 | cg06761424 | KIAA1210 | TSS1500 | Island | NA | II | X |
| 772 | cg06774787 | KLF8; KLF8; KLF8; KL | 5′UTR; 1stExon; 1st | Island | NA | I | X |
| | | F8 | Exon; 5′UTR | | | | |
| 773 | cg06782686 | NID2 | TSS200 | Island | NA | I | 14 |
| 774 | cg06793562 | | | Island | NA | II | 6 |
| 775 | cg06805513 | SLC16A2 | Body | Island | NA | I | X |
| 776 | cg06811467 | ATP8B2; ATP8B2 | TSS1500; 5′UTR | N_Shore | NA | II | 1 |
| 777 | cg06817490 | | | | TRUE | I | 17 |
| 778 | cg06833110 | MEIS1 | Body | Island | NA | I | 2 |
| 779 | cg06836736 | ME1 | TSS1500 | Island | NA | II | 6 |
| 780 | cg06845853 | WIT1 | Body | S_Shore | NA | I | 11 |
| 781 | cg06915321 | ZIC3 | TSS200 | Island | NA | I | X |
| 782 | cg06915773 | PTN | 5′UTR | | NA | II | 7 |
| 783 | cg06916239 | PAX3; PAX3; PAX3; C | Body; Body; Body; 5 | N_Shore | NA | I | 2 |
| | | CDC140; CCDC140; P | ′UTR; 1stExon; Bod | | | | |
| | | AX3; PAX3; PAX3; PA | y; Body; Body; Body | | | | |
| | | X3; PAX3 | ; Body | | | | |
| 784 | cg06930722 | CHST11 | Body | | NA | I | 12 |
| 785 | cg06943420 | MMP23A; MMP23B | TSS1500; TSS1500 | Island | TRUE | I | 1 |
| 786 | cg06943912 | 5LC44A2 | Body | S_Shelf | NA | I | 19 |
| 787 | cg06960356 | KCNQ1; KCNQ1 | Body; Body | Island | NA | I | 11 |
| 788 | cg06962970 | NTM | Body | | TRUE | II | 11 |
| 789 | cg06963233 | ANPEP | 5′UTR | Island | NA | II | 15 |
| 790 | cg06969287 | TTC28 | Body | | TRUE | II | 22 |
| 791 | cg06970190 | | | Island | NA | II | 3 |
| 792 | cg06971227 | AATK | Body | Island | NA | II | 17 |
| 793 | cg06975914 | | | | TRUE | II | 1 |
| 794 | cg06976096 | | | Island | TRUE | II | 3 |
| 795 | cg06982190 | | | Island | NA | II | 7 |
| 796 | cg06991484 | | | S_Shore | NA | II | 8 |
| 797 | cg07017114 | DLA-II | Body | Island | NA | I | 6 |
| 798 | cg07019438 | EGLN3 | Body | Island | NA | I | 14 |
| 799 | cg07022764 | ASPG | Body | Island | NA | II | 14 |
| 800 | cg07034362 | ALDH1L1 | TSS200 | Island | TRUE | I | 3 |
| 801 | cg07035145 | PYGL; PYGL | Body; Body | Island | NA | I | 14 |
| 802 | cg07037019 | OR10J5 | TSS1500 | | NA | II | 1 |
| 803 | cg07037371 | | | | TRUE | I | 4 |
| 804 | cg07039423 | KCP; KCP | Body; Body | Island | NA | I | 7 |
| 805 | cg07044859 | | | Island | NA | I | 10 |
| 806 | cg07045330 | | | Island | NA | II | 3 |
| 807 | cg07050616 | FLJ41350 | Body | Island | TRUE | II | 10 |
| 808 | cg07053014 | SLC29A1; SLC29A1 | TSS200; TSS200 | N_Shore | NA | II | 6 |
| 809 | cg07077013 | | | Island | NA | II | 2 |
| 810 | cg07083818 | KIAA1683; KIAA1683 | TSS1500; TSS1500 | | TRUE | II | 19 |
| | | ; KIAAI683 | ; TSS1500 | | | | |
| 811 | cg07101841 | SEPT9; SEPT9; SEPT9 | 5′UTR; TSS200; Bo | | TRUE | I | 17 |
| | | | dy | | | | |
| 812 | cg07109801 | MIR425; DALRD3; ND | Body; 5′UTR; TSS1 | N_Shore | NA | II | 3 |
| | | UFAF3; NDUFAF3; N | 500; TSS1500; TSS | | | | |
| | | DUFAF3; NDUFAF3 | 1500; TSS1500 | | | | |
| 813 | cg07137955 | | | Island | TRUE | I | 5 |
| 814 | cg07147449 | | | Island | NA | I | 12 |
| 815 | cg07173635 | | | Island | NA | II | 9 |
| 816 | cg07177380 | | | N_Shelf | TRUE | I | 4 |
| 817 | cg07185131 | | | | TRUE | I | 2 |
| 818 | cg07190535 | PDE4D; PDE4D | Body; TSS1500 | Island | TRUE | I | 5 |
| 819 | cg07197785 | | | Island | TRUE | I | 1 |
| 820 | cg07200280 | RFTN1 | 5′UTR | Island | NA | II | 3 |
| 821 | cg07211140 | WT1; WT1; WT1; WT1 | Body; Body; Body; | Island | NA | I | 11 |
| | | | Body | | | | |
| 822 | cg07219542 | GSTM2; GSTM2 | 1stExon; 1stExon | Island | TRUE | I | 1 |
| 823 | cg07239592 | POU6F2; POU6F2 | Body; Body | Island | NA | II | 7 |
| 824 | cg07262247 | PDLIM4; PDLIM4 | Body; Body | Island | NA | I | 5 |
| 825 | cg07271361 | STK19; STKI9; DOM3 | Body; Body; TSS15 | S_Shore | NA | II | 6 |
| | | Z; STK19 | 00; Body | | | | |
| 826 | cg07283795 | | | | TRUE | II | 5 |
| 827 | cg07291445 | | | Island | NA | I | 7 |
| 828 | cg07302069 | HOXA7; HOXA7 | 5′UTR; 1stExon | Island | NA | I | 7 |
| 829 | cg07335294 | SNAP91; SNAP91 | Body; 5′UTR | Island | TRUE | II | 6 |
| 830 | cg07349208 | | | Island | TRUE | I | 4 |
| 831 | cg07367113 | BRD9; BRD9; BRD9 | Body; Body; Body | N_Shore | NA | II | 5 |
| 832 | cg07368796 | | | N_Shore | NA | I | 10 |
| 833 | cg07376381 | LCK; LCK | Body; Body | Island | NA | I | 1 |
| 834 | cg07378762 | MECOM | Body | N_Shelf | TRUE | II | 3 |
| 835 | cg07395354 | MEOX2 | TSS1500 | | TRUE | II | 7 |
| 836 | cg07402669 | APCDD1L | TSS200 | Island | NA | I | 20 |
| 837 | cg07416656 | TLX1 | Body | Island | TRUE | I | 10 |
| 838 | cg07433663 | MEIS2; MEIS2; MEIS2 | 1stExon; Body; Bod | Island | NA | I | 15 |
| | | ; MEIS2; MEIS2; MEIS | y; 5′UTR; Body; Bod | | | | |
| | | 2; MEIS2 | y; Body | | | | |
| 839 | cg07443748 | CCT8L2; CCT8L2 | 1stExon; 5′UTR | | NA | II | 22 |
| 840 | cg07448060 | MAGI2 | TSS1500 | Island | NA | I | 7 |
| 841 | cg07459478 | VOPP1 | Body | | TRUE | I | 7 |
| 842 | cg07475654 | PNKD; TMBIM1 | Body; 5′UTR | | TRUE | II | 2 |
| 843 | cg07485357 | ADCY4 | Body | Island | NA | I | 14 |
| 844 | cg07486252 | ANK2; ANK2; ANK2 | Body; Body; Body | | TRUE | II | 4 |
| 845 | cg07525299 | ASCL2 | TSS1500 | S_Shore | NA | I | 11 |
| 846 | cg07548313 | CD1C | Body | | NA | II | 1 |
| 847 | cg07548607 | ZSWIM2 | TSS200 | Island | TRUE | I | 2 |
| 848 | cg07551545 | FBXO17; FBXO17 | 5′UTR; 1stExon | Island | TRUE | I | 19 |
| 849 | cg07560096 | ME3; ME3; ME3; ME3 | 5′UTR; 1stExon; 5′U | Island | NA | II | 11 |
| | | | TR; 5′UTR | | | | |
| 850 | cg07564962 | | | Island | NA | I | 5 |
| 851 | cg07582955 | KLHL17 | Body | Island | NA | II | 1 |
| 852 | cg07587796 | ENPEP; ENPEP | 1stExon; 5′UTR | N_Shore | NA | II | 4 |
| 853 | cg07603330 | | | Island | NA | I | 11 |
| 854 | cg07620844 | RBM15B | 1stExon | N_Shore | NA | II | 3 |
| 855 | cg07627556 | | | S_Shore | TRUE | I | 11 |
| 856 | cg07629454 | CASR | 5′UTR | Island | NA | I | 3 |
| 857 | cg07641160 | FAM162B; FAM162B | 1stExon; 5′UTR | Island | NA | I | 6 |
| 858 | cg07644368 | CDO1 | TSS1500 | S_Shore | NA | II | 5 |
| 859 | cg07647825 | SLC46A3; SLC46A3 | TSS1500; TSS1500 | S_Shore | NA | II | 13 |
| 860 | cg07662771 | PSMD6 | Body | N_Shore | NA | I | 3 |
| 861 | cg07690882 | | | Island | NA | II | 11 |
| 862 | cg07697177 | C1orf86; LOC1001280 | Body; TSS200; Bod | N_Shore | NA | II | 1 |
| | | 03; C1orf86 | y | | | | |
| 863 | cg07697895 | WNT2; WNT2; WNT2 | Body; 5′UTR; 1stEx | Island | NA | I | 7 |
| | | | on | | | | |
| 864 | cg07702750 | TRIL | TSS200 | Island | NA | I | 7 |
| 865 | cg07758529 | IL1RAPL2 | 5′UTR | S_Shore | NA | I | X |
| 866 | cg07763047 | GABRB2; GABRB2 | 5′UTR; 5′UTR | Island | NA | I | 5 |
| 867 | cg07774424 | | | | NA | II | X |
| 868 | cg07776847 | C4orf49 | Body | Island | NA | II | 4 |
| 869 | cg07777008 | FIBIN; FIBIN | 5′UTR; 1stExon | | TRUE | I | 11 |
| 870 | cg07792478 | MIR124-2 | TSS1500 | Island | NA | I | 8 |
| 871 | cg07809589 | MEIS1 | Body | S_Shelf | NA | II | 2 |
| 872 | cg07810164 | EGFL6; EGFL6 | Body; Body | Island | TRUE | I | X |
| 873 | cg07810726 | EBF1 | Body | N_Shore | NA | II | 5 |
| 874 | cg07811054 | | | | NA | II | 15 |
| 875 | cg07811110 | SLC6A15; SLC6A15; S | TSS1500; TSS1500 | S_Shore | NA | I | 12 |
| | | LC6A15 | ; TSS1500 | | | | |
| 876 | cg07812877 | RGL3; RGL3; CCDC15 | TSS1500; TSS1500 | Island | NA | I | 19 |
| | | 1 | ; 3′UTR | | | | |
| 877 | cg07855639 | CDC42EP1 | 5′UTR | S_Shore | TRUE | I | 22 |
| 878 | cg07863022 | SEPT9; SEPT9; SEPT9 | 5′UTR; Body; TSS1 | | NA | I | 17 |
| | | | 500 | | | | |
| 879 | cg07876898 | CCDC65 | TSS200 | | TRUE | II | 12 |
| 880 | cg07887243 | MAOB | 1stExon | Island | NA | I | X |
| 881 | cg07891531 | | | Island | TRUE | I | 16 |
| 882 | cg07903237 | | | N_Shore | NA | II | 1 |
| 883 | cg07907386 | PHOX2B | Body | Island | TRUE | I | 4 |
| 884 | cg07911353 | KIAA1549; KIAA1549 | Body; Body | | TRUE | II | 7 |
| 885 | cg07915434 | TLX1 | Body | Island | TRUE | I | 10 |
| 886 | cg07916654 | GPR123 | Body | N_Shore | NA | I | 10 |
| 887 | cg07917150 | HOXA3; HOXA3; HO | 5′UTR; TSS1500; 5 | N_Shore | NA | II | 7 |
| | | XA3 | UTR | | | | |
| 888 | cg07921314 | NRF1 | 5′UTR | S_Shore | NA | II | 7 |
| 889 | cg07964113 | RPTOR; RPTOR | Body; Body | Island | NA | I | 17 |
| 890 | cg07965640 | SPAG16; SPAG16 | Body; Body | S_Shore | NA | II | 2 |
| 891 | cg07969619 | | | Island | NA | II | 12 |
| 892 | cg07973435 | SIM1 | Body | Island | NA | I | 6 |
| 893 | cg07976064 | | | Island | NA | I | 11 |
| 894 | cg08008403 | LHX9 | Body | S_Shore | NA | II | 1 |
| 895 | cg08021564 | SPOCK2; SPOCK2 | Body; Body | Island | NA | II | 10 |
| 896 | cg08023416 | IL1RL2 | 5′UTR | Island | TRUE | II | 2 |
| 897 | cg08034379 | WDR69 | TSS200 | Island | TRUE | I | 2 |
| 898 | cg08036309 | | | N_Shore | TRUE | I | 4 |
| 899 | cg08045824 | HOXC12 | Body | S_Shore | NA | I | 12 |
| 900 | cg08048577 | TPP2 | Body | S_Shore | NA | II | 13 |
| 901 | cg08055330 | C9orf86; C9orf86; LOC | Body; Body; TSS15 | S_Shore | NA | II | 9 |
| | | 100131193 | 00 | | | | |
| 902 | cg08084339 | | | | TRUE | II | 11 |
| 903 | cg08084675 | QSER1 | 5′UTR | Island | NA | II | 11 |
| 904 | cg08089364 | | | N_Shelf | NA | II | 6 |
| 905 | cg08100687 | | | Island | TRUE | II | 13 |
| 906 | cg08101036 | HOXA3; HOXA3; HO | 5′UTR; 5′UTR; TSS | N_Shore | NA | I | 7 |
| | | XA3 | 200 | | | | |
| 907 | cg08128826 | LHX9; LHX9 | Body; Body | | NA | I | 1 |
| 908 | cg08140548 | SFRS11 | Body | | NA | I | 1 |
| 909 | cg08141495 | NOL4 | Body | Island | NA | II | 18 |
| 910 | cg08142344 | GSTM2; GSTM2; GST | 5′UTR; 1stExon; 1st | Island | TRUE | II | 1 |
| | | M2; GSTM2 | Exon; 5′UTR | | | | |
| 911 | cg08146483 | LHX8 | Body | Island | NA | I | 1 |
| 912 | cg08151731 | FBN1 | TSS1500 | Island | NA | II | 15 |
| 913 | cg08152546 | | | Island | NA | II | 13 |
| 914 | cg08154107 | ECE2; ECE2; ECE2; EC | Body; Body; Body; | Island | TRUE | I | 3 |
| | | E2 | Body | | | | |
| 915 | cg08158862 | NR5A2; NR5A2 | Body; Body | S_Shore | NA | II | 1 |
| 916 | cg08161323 | CXCL1 | TSS1500 | N_Shore | NA | II | 4 |
| 917 | cg08162372 | BMP4; BMP4 | 5′UTR; 5′UTR | Island | NA | I | 14 |
| 918 | cg08164233 | RFXAP | Body | S_Shore | NA | II | 13 |
| 919 | cg08167951 | PON3 | 1stExon | Island | NA | I | 7 |
| 920 | cg08168585 | | | N_Shelf | NA | II | 16 |
| 921 | cg08168768 | KIAA1210 | TSS1500 | Island | NA | II | X |
| 922 | cg08186341 | SF3B3; SNORD111B | Body; TSS1500 | | NA | I | 16 |
| 923 | cg08188069 | SDR16C6 | TSS1500 | | NA | II | 8 |
| 924 | cg08190858 | | | Island | NA | I | 12 |
| 925 | cg08196032 | | | N_Shore | NA | II | 14 |
| 926 | cg08214060 | NTM; NTM; NTM; NT | Body; Body; Body; | | NA | II | 11 |
| | | M | Body | | | | |
| 927 | cg08217024 | MDGA2 | TSS1500 | Island | NA | I | 14 |
| 928 | cg08231710 | MMP23A; MMP23B | TSS1500; TSS1500 | Island | TRUE | I | 1 |
| 929 | cg08233811 | FLJ22536 | Body | S_Shore | NA | II | 6 |
| 930 | cg08238568 | RGS7 | Body | | TRUE | II | 1 |
| 931 | cg08239375 | SLC4AIAP; SUPT7L | Body; TSS1500 | S_Shore | NA | II | 2 |
| 932 | cg08263589 | | | Island | NA | I | 3 |
| 933 | cg08266417 | AOX1 | TSS200 | Island | TRUE | I | 2 |
| 934 | cg08272731 | LHX8 | Body | Island | NA | I | 1 |
| 935 | cg08320659 | DFE; HFE; DFE; DFE; H | Body; Body; Body; | | TRUE | II | 6 |
| | | FE; DFE; DFE; DFE; HF | Body; Body; Body; | | | | |
| | | E | Body; Body; Body | | | | |
| 936 | cg08322102 | ZEHX4; LOC10019237 | 5′UTR; Body | S_Shore | NA | I | 8 |
| | | 8 | | | | | |
| 937 | cg08332074 | | | Island | NA | II | 16 |
| 938 | cg08344081 | | | Island | NA | I | 6 |
| 939 | cg08348649 | FAM70A; FAM70A; F | Body; Body; Body | N_Shore | NA | II | X |
| | | AM70A | | | | | |
| 940 | cg08350814 | C2orf88; C2orf88; C2or | TSS1500; 5′UTR; T | Island | TRUE | I | 2 |
| | | 188; C2orf88 | SS1500; 5′UTR | | | | |
| 941 | cg08368617 | | | Island | TRUE | I | 6 |
| 942 | cg08368654 | | | Island | TRUE | I | 6 |
| 943 | cg08382235 | TBX1; TBX1; TBX1 | Body; Body; Body | Island | TRUE | I | 22 |
| 944 | cg08387285 | ME3; ME3; ME3 | | | | | |
| | 5′UTR; TSS | Island | NA | I | 11 | 86383252 | GGCAAAGCGA |
| | 200; 5′UTR | | | | | | AACCAAGCCC |
| | | | | | | | TGGAAGCAGA |
| | | | | | | | TCCGGTGCGG |
| | | | | | | | GTGACAGCCG |
| 945 | cg08441803 | SKAPLSKAP1 | Body; Body | | NA | I | 17 |
| 946 | cg08443605 | | | S_Shore | NA | I | 3 |
| 947 | cg08445802 | | | Island | NA | I | 6 |
| 948 | cg08446038 | ITGA2 | Body | | TRUE | II | 5 |
| 949 | cg08455778 | LOC645323 | Body | N_Shore | NA | I | 5 |
| 950 | cg08462988 | | | | TRUE | I | 3 |
| 951 | cg08464190 | CBLN1 | 1stExon | Island | TRUE | I | 16 |
| 952 | cg08474859 | | | Island | NA | I | 4 |
| 953 | cg08477594 | ME3; ME3; ME3; ME3 | TSS200; TSS1500; | Island | NA | II | 11 |
| | | | 1stExon; 5′UTR | | | | |
| 954 | cg08479865 | DRD3; DRD3; DRD3; D | 1stExon; 5′UTR; 1st | | TRUE | II | 3 |
| | | RD3 | Exon; 5′UTR | | | | |
| 955 | cg08487063 | TLX1 | Body | Island | TRUE | I | 10 |
| 956 | cg08513547 | LOC134466 | Body | N_Shelf | NA | II | 5 |
| 957 | cg08516516 | CDO1 | TSS200 | Island | NA | I | 5 |
| 958 | cg08518715 | PARK2; PARK2; PAR | Body; Body; Body | | NA | II | 6 |
| | | K2 | | | | | |
| 959 | cg08521073 | HRK | 3′UTR | N_Shelf | NA | II | 12 |
| 960 | cg08530237 | | | Island | NA | II | 12 |
| 961 | cg08546494 | | | Island | NA | II | 2 |
| 962 | cg08548396 | | | Island | NA | I | 6 |
| 963 | cg08552198 | | | Island | TRUE | II | 16 |
| 964 | cg08553437 | TMEM155; LOC10019 | TSS200; Body | Island | NA | I | 4 |
| | | 2379 | | | | | |
| 965 | cg08554295 | PDE4D; PDE4D | Body; TSS1500 | S_Shore | NA | I | 5 |
| 966 | cg08560874 | HCG4 | Body | Island | NA | II | 6 |
| 967 | cg08567279 | LECT1; LECT1 | Body; Body | Island | TRUE | I | 13 |
| 968 | cg08575537 | EPO | Body | Island | TRUE | I | 7 |
| 969 | cg08585897 | KARS; TERF2IP; KAR | 5′UTR; TSS1500; B | N_Shore | NA | II | 16 |
| | | S | ody | | | | |
| 970 | cg08596308 | ATP6V1G2; NFKB1L1 | TSS1500; Body; Bo | | NA | II | 6 |
| | | ; NFKBIL1; NFKBIL1; | dy; Body; TSS1500; | | | | |
| | | ATP6V1G2; NFKBIL1 | Body | | | | |
| 971 | cg08604097 | NXPH1 | Body | N_Shore | NA | I | 7 |
| 972 | cg08605656 | SLC16A2 | Body | S_Shore | NA | I | X |
| 973 | cg08612384 | DHRS13 | Body | N_Shore | NA | II | 17 |
| 974 | cg08614481 | HTR1B | TSS200 | Island | NA | II | 6 |
| 975 | cg08620044 | LOC645323 | TSS1500 | Island | NA | I | 5 |
| 976 | cg08628006 | | | S_Shelf | NA | I | 13 |
| 977 | cg08673716 | | | Island | NA | II | 3 |
| 978 | cg08691567 | KRT3 | Body | Island | NA | I | 12 |
| 979 | cg08700190 | SRD5A1 | Body | S_Shore | TRUE | II | 5 |
| 980 | cg08726446 | | | Island | TRUE | II | 12 |
| 981 | cg08728495 | | | | NA | I | 12 |
| 982 | cg08736638 | | | N_Shore | NA | II | 20 |
| 983 | cg08760398 | FAM70A; FAM70A; F | Body; Body; Body | N_Shore | NA | II | X |
| | | AM70A | | | | | |
| 984 | cg08767182 | | | Island | NA | II | 5 |
| 985 | cg08772837 | PCDHA7; PCDHAC2; | Body; TSS1500; Bo | N_Shore | NA | II | 5 |
| | | PCDHAl2; PCDHA6; P | dy; Body; Body; Bod | | | | |
| | | CDHA10; PCDHA4; P | y; Body; Body; Body | | | | |
| | | CDHA11; PCDHA8; P | ; Body; Body; Body; | | | | |
| | | CDHA6; PCDHA1; PC | Body; Body; Body; | | | | |
| | | DHA2; PCDHA1; PCD | Body; Body; Body | | | | |
| | | HA9; PCDHA13; PCD | | | | | |
| | | HA5; PCDHAC1; PCD | | | | | |
| | | HA3 PCDHA10 | | | | | |
| 986 | cg08783090 | GABRE; GABRE | 1stExon; 5′UTR | Island | NA | I | X |
| 987 | cg08786077 | QPCT | TSS200 | Island | TRUE | I | 2 |
| 988 | cg08799778 | LMF1 | Body | N_Shelf | NA | II | 16 |
| 989 | cg08812189 | ZIC4; ZIC4; ZIC4; ZIC4 | Body; TSS200; TSS | Island | TRUE | II | 3 |
| | | ; ZIC4 | 200; Body; Body | | | | |
| 990 | cg08814148 | | | Island | TRUE | II | X |
| 991 | cg08827307 | ADAM32 | TSS200 | Island | NA | II | 8 |
| 992 | cg08827358 | BEAN | 5′UTR | Island | NA | II | 16 |
| 993 | cg08832906 | CLEC2L | 1stExon | Island | NA | I | 7 |
| 994 | cg08844169 | TMEM39A | Body | | NA | II | 3 |
| 995 | cg08846245 | SLC39A7; RXRB; SLC | Body; TSS1500; Bo | S_Shore | NA | II | 6 |
| | | 39A7 | dy | | | | |
| 996 | cg08856601 | DNAJC6 | TSS200 | N_Shore | NA | II | 1 |
| 997 | cg08876932 | PHOX2A | TSS1500 | Island | NA | I | 11 |
| 998 | cg08883223 | LHX9; LHX9 | Body; Body | N_Shore | NA | I | 1 |
| 999 | cg08884974 | | | N_Shelf | NA | II | 15 |
| 1000 | cg08886154 | PAX4 | TSS1500 | | NA | II | 7 |
| 1001 | cg08886651 | TBX1; TBX1; TBX1 | TSS1500; TSS1500 | Island | NA | I | 22 |
| | | | ; TSS1500 | | | | |
| 1002 | cg08893839 | | | Island | TRUE | I | 5 |
| 1003 | cg08897924 | CC2D1B | TSS1500 | N_Shore | NA | II | 1 |
| 1004 | cg08898155 | PON3 | TSS1500 | Island | NA | I | 7 |
| 1005 | cg08924203 | MX1; MX1 | 5′UTR; 5′UTR | Island | NA | II | 21 |
| 1006 | cg08925169 | TRIM34; TRIM78P; TR | Body; TSS200; Bod | | NA | II | 11 |
| | | IM34; TRIM34; TRIM6 | y; Body; Body | | | | |
| | | -TRIM34 | | | | | |
| 1007 | cg08947084 | SNORD47; GAS5; SNO | TSS1500; Body; TS | N_Shelf | NA | II | 1 |
| | | RD81; SNORD80 | S1500; TSS200 | | | | |
| 1008 | cg08952506 | AOX1 | TSS200 | Island | TRUE | II | 2 |
| 1009 | cg08954277 | | | S_Shore | NA | I | 4 |
| 1010 | cg08955941 | | | | NA | II | 5 |
| 1011 | cg08958015 | CCDC65; CCDC65 | 1stExon; 5′UTR | | TRUE | II | 12 |
| 1012 | cg08958294 | GRM1; GRM1 | 5′UTR; 5′UTR | N_Shore | NA | I | 6 |
| 1013 | cg08993172 | ALX4 | Body | Island | NA | II | 11 |
| 1014 | cg09006039 | | | | TRUE | II | 4 |
| 1015 | cg09017619 | HOXA7 | TSS200 | Island | NA | I | 7 |
| 1016 | cg09019936 | | | N_Shore | TRUE | II | 2 |
| 1017 | cg09031836 | | | N_Shelf | NA | II | X |
| 1018 | cg09041207 | FBXO31; FBXO31 | TSS1500; Body | Island | NA | II | 16 |
| 1019 | cg09065413 | PTCH2 | 3′UTR | | TRUE | II | 1 |
| 1020 | cg09066883 | PTPRN2; PTPRN2; PT | Body; Body; Body | | NA | I | 7 |
| | | PRN2 | | | | | |
| 1021 | cg09072216 | SPG20; SPG20; SPG20; | 5′UTR; 5′UTR; 5′UT | N_Shore | NA | II | 13 |
| | | SPG20 | R; 5′UTR | | | | |
| 1022 | cg09076431 | LHX9; LHX9 | Body; Body | N_Shore | NA | II | 1 |
| 1023 | cg09080553 | | | N_Shore | NA | I | 7 |
| 1024 | cg09092713 | | | Island | NA | II | X |
| 1025 | cg09094964 | | | | TRUE | I | 12 |
| 1026 | cg09100593 | SMC4; SMC4; MIR16- | Body; Body; TSS20 | S_Shelf | NA | II | 3 |
| | | 2 | 0 | | | | |
| 1027 | cg09109450 | C2orf43 | 5′UTR | Island | NA | II | 2 |
| 1028 | cg09116618 | | | | TRUE | II | 6 |
| 1029 | cg09136346 | | | Island | TRUE | I | 20 |
| 1030 | cg09151979 | | | Island | NA | I | 6 |
| 1031 | cg09159022 | CSMD1 | Body | Island | NA | II | 8 |
| 1032 | cg09171882 | GLIPR1 | TSS200 | | NA | II | 12 |
| 1033 | cg09211763 | COL5A2; COL5A2 | 1stExon; 5′UTR | | NA | II | 2 |
| 1034 | cg09220563 | | | | TRUE | II | 14 |
| 1035 | cg09226388 | BMP4; BMP4; BMP4 | 5′UTR; 5′UTR; TSS | Island | NA | I | 14 |
| | | | 1500 | | | | |
| 1036 | cg09234698 | | | | TRUE | II | 1 |
| 1037 | cg09236658 | | | N_Shore | NA | II | 6 |
| 1038 | cg09238801 | PNMA1; PNMA1 | 1stExon; 3′UTR | N_Shore | NA | I | 14 |
| 1039 | cg09260773 | TBX15 | 5′UTR | N_Shore | NA | II | 1 |
| 1040 | cg09275956 | | | S_Shelf | TRUE | I | 1 |
| 1041 | cg09309208 | | | | TRUE | II | 13 |
| 1042 | cg09327714 | | | Island | NA | I | 1 |
| 1043 | cg09356916 | | | | TRUE | II | 4 |
| 1044 | cg09364020 | | | S_Shore | NA | II | 6 |
| 1045 | cg09372386 | DNAH10 | Body | | NA | II | 12 |
| 1046 | cg09393254 | MCHR2; MCHR2 | TSS200; TSS200 | Island | TRUE | I | 6 |
| 1047 | cg09406238 | MTMR8 | TSS200 | Island | NA | I | X |
| 1048 | cg09411719 | | | | NA | I | 6 |
| 1049 | cg09418984 | MYADM | TSS200 | Island | NA | I | 19 |
| 1050 | cg09423283 | GRM1; GRM1 | 3′UTR; Body | N_Shore | NA | I | 6 |
| 1051 | cg09435415 | DPYS | TSS200 | S_Shore | NA | II | 8 |
| 1052 | cg09441363 | KCNK2; KCNK2; KCN | TSS1500; TSS1500 | Island | NA | I | 1 |
| | | K2 | ; Body | | | | |
| 1053 | cg09443457 | | | S_Shore | NA | II | X |
| 1054 | cg09464206 | CASP8; CASP8; CASP | 5′UTR; 5′UTR; 5′UT | | NA | II | 2 |
| | | 8; CASP8; CASP8 | R; TSS1500; 5′UTR | | | | |
| 1055 | cg09469566 | | | Island | NA | I | 13 |
| 1056 | cg09491897 | RPTOR; RPTOR | Body; Body | Island | NA | I | 17 |
| 1057 | cg09501509 | | | | TRUE | II | 6 |
| 1058 | cg09502069 | DEGS1; DEGS1 | Body; Body | S_Shore | NA | I | 1 |
| 1059 | cg09506473 | PSKH2 | TSS200 | Island | NA | I | 8 |
| 1060 | cg09508289 | | | | TRUE | II | 6 |
| 1061 | cg09519327 | ATG9B; NOS3 | 3′UTR; Body | Island | NA | I | 7 |
| 1062 | cg09521647 | AFF3; AFF3 | TSS1500; 5′UTR | S_Shore | TRUE | II | 2 |
| 1063 | cg09537620 | PAX6; PAX6; PAX6 | Body; Body; Body | Island | NA | I | 11 |
| 1064 | cg09544050 | BAI1 | Body | Island | TRUE | I | 8 |
| 1065 | cg09544728 | PRKAR1B; PRKAR1B | Body; Body; Body; | Island | NA | I | 7 |
| | | ; PRKAR1B; PRKAR1 | Body; Body; Body | | | | |
| | | B; PRKAR1B; PRKAR | | | | | |
| | | 1B | | | | | |
| 1066 | cg09558195 | GRM1; GRM1 | 5′UTR; 5′UTR | Island | NA | I | 6 |
| 1067 | cg09558547 | | | | NA | II | 6 |
| 1068 | cg09577324 | OR1OA2 | TSS200 | | NA | II | 11 |
| 1069 | cg09579623 | | | N_Shelf | NA | II | 6 |
| 1070 | cg09607047 | MIR365-1 | TSS1500 | | TRUE | II | 16 |
| 1071 | cg09610569 | SLITRK2; SLITRK2; S | 5′UTR; 5′UTR; 5′UT | Island | NA | I | X |
| | | LITRK2; SLITRK2 | R; 5′UTR | | | | |
| 1072 | cg09627874 | | | N_Shore | TRUE | I | 12 |
| 1073 | cg09632273 | DDAH2 | Body | Island | NA | I | 6 |
| 1074 | cg09633973 | HIC1 | TSS1500 | Island | NA | I | 17 |
| 1075 | cg09640202 | IMP4 | Body | S_Shore | NA | II | 2 |
| 1076 | cg09645888 | ME3; ME3; ME3; ME3 | TSS200; TSS1500; | Island | NA | II | 11 |
| | | | 1stExon; 5′UTR | | | | |
| 1077 | cg09658429 | | | Island | NA | II | 12 |
| 1078 | cg09670128 | KRT7; KRT7 | 5′UTR; 1stExon | Island | NA | I | 12 |
| 1079 | cg09675542 | | | | TRUE | II | 7 |
| 1080 | cg09704166 | PAX8; PAX8; PAX8; P | Body; Body; Body; | N_Shore | NA | II | 2 |
| | | AX8; PAX8; LOC4408 | Body; Body; Body | | | | |
| | | 39 | | | | | |
| 1081 | cg09715059 | | | | TRUE | II | 6 |
| 1082 | cg09729613 | AOX1 | TSS200 | Island | TRUE | I | 2 |
| 1083 | cg09734791 | MSC | 1stExon | Island | NA | I | 8 |
| 1084 | cg09760963 | RNF175 | Body | Island | NA | I | 4 |
| 1085 | cg09781936 | TBX1; TBX1; TBX1 | Body; Body; Body | N_Shore | NA | II | 22 |
| 1086 | cg09786278 | RGS7 | Body | | TRUE | II | 1 |
| 1087 | cg09789590 | HIF3A; HIF3A | Body; TSS1500 | Island | NA | II | 19 |
| 1088 | cg09806262 | ALOX5 | Body | S_Shore | TRUE | II | 10 |
| 1089 | cg09829319 | GCM2 | TSS200 | N_Shore | NA | II | 6 |
| 1090 | cg09839443 | QPCT | TSS200 | Island | TRUE | II | 2 |
| 1091 | cg09849405 | CMTM1; CMTM1; CM | 3′UTR; 3′UTR; TSS | Island | NA | I | 16 |
| | | TM2; CMTM1; CMTM | 1500; 3′UTR; 3′UTR | | | | |
| | | 1; CMTM1; CMTM1; C | ; 3′UTR; 3′UTR; 3′U | | | | |
| | | MTM1; CMTM1 | TR; 3′UTR | | | | |
| 1092 | cg09866743 | ARPP-21 | 5′UTR | | NA | I | 3 |
| 1093 | cg09871637 | LMO4 | Body | N_Shore | NA | II | 1 |
| 1094 | cg09884341 | LOC645323; LOC6453 | Body; TSS1500 | N_Shore | TRUE | II | 5 |
| | | 23 | | | | | |
| 1095 | cg09903452 | SEPT9; SEPT9; SEPT9 | 5′UTR; Body; TSS1 | | NA | II | 17 |
| | | | 500 | | | | |
| 1096 | cg09919570 | | | Island | NA | I | 16 |
| 1097 | cg09941406 | | | S_Shore | NA | I | 12 |
| 1098 | cg09957864 | GPT | TSS1500 | N_Shore | NA | I | 8 |
| 1099 | cg09970569 | RSPO2 | Body | N_Shore | NA | II | 8 |
| 1100 | cg09970593 | OPRD1 | Body | S_Shore | NA | II | 1 |
| 1101 | cg09971314 | | | | NA | I | 6 |
| 1102 | cg09972569 | | | Island | NA | I | 2 |
| 1103 | cg09979311 | HN1L | Body | S_Shore | NA | I | 16 |
| 1104 | cg09993945 | CCAR1 | Body | S_Shore | NA | II | 10 |
| 1105 | cg10021724 | | | | NA | II | 13 |
| 1106 | cg10042106 | OTX2 | 5′UTR | Island | NA | I | 14 |
| 1107 | cg10050661 | KRT222 | TSS200 | | NA | II | 17 |
| 1108 | cg10054197 | FAM3B; FAM3B | Body; Body | Island | NA | I | 21 |
| 1109 | cg10067182 | HDAC4 | 3′UTR | Island | NA | I | 2 |
| 1110 | cg10069493 | ALOX5 | Body | Island | TRUE | I | 10 |
| 1111 | cg10106804 | GJA4 | TSS200 | Island | NA | I | 1 |
| 1112 | cg10109351 | | | N_Shore | TRUE | I | 3 |
| 1113 | cg10122477 | GLBIL3 | Body | S_Shore | NA | II | 11 |
| 1114 | cg10143323 | | | | TRUE | II | 11 |
| 1115 | cg10143811 | LMO3; LMO3 | 5′UTR; 5′UTR | | NA | I | 12 |
| 1116 | cg10160397 | ME3; ME3; ME3; ME3 | TSS200; TSS1500; | Island | NA | II | 11 |
| | | | 1stExon; 5′UTR | | | | |
| 1117 | cg10174333 | | | | NA | II | 1 |
| 1118 | cg10182317 | CLVS2 | TSS200 | Island | NA | I | 6 |
| 1119 | cg10223827 | NRN1; NRN1 | 5′UTR; 1stExon | Island | TRUE | I | 6 |
| 1120 | cg10233654 | DUSP6; DUSP6 | Body; Body | N_Shore | NA | I | 12 |
| 1121 | cg10244666 | WT1; WT1; WT1; WT1 | Body; Body; Body; | | NA | I | 11 |
| | | | Body | | | | |
| 1122 | cg10249375 | | | Island | TRUE | I | 1 |
| 1123 | cg10259748 | C6orf186 | Body | Island | TRUE | I | 6 |
| 1124 | cg10263682 | SOX2OT | Body | Island | TRUE | I | 3 |
| 1125 | cg10264687 | | | N_Shore | TRUE | II | 16 |
| 1126 | cg10271061 | ATP6V1G2; NFKBIL1 | TSS1500; Body; Bo | | NA | I | 6 |
| | | ; NFKB1L1; NFKBIL1; | dy; Body; TSS1500; | | | | |
| | | ATP6V1G2; NFKBIL1 | Body | | | | |
| 1127 | cg10280007 | FGFR2; FGFR2; FGFR | Body; Body; Body; | | TRUE | II | 10 |
| | | 2; FGFR2; FGFR2; FGF | Body; Body; Body; | | | | |
| | | R2; FGFR2; FGFR2; FG | Body; Body; Body | | | | |
| | | FR2 | | | | | |
| 1128 | cg10289744 | CHL1 | 5′UTR | | TRUE | II | 3 |
| 1129 | cg10303487 | DPYS | 1stExon | Island | NA | I | 8 |
| 1130 | cg10308673 | | | | TRUE | I | 11 |
| 1131 | cg10314133 | C1orf86; LOC1001280 | Body; TSS200; Bod | N_Shore | NA | II | 1 |
| | | 03; C1orf86 | y | | | | |
| 1132 | cg10320410 | | | | NA | I | 7 |
| 1133 | cg10321196 | LOC100303728; SLC2 | Body; TSS1500 | N_Shore | NA | II | X |
| | | 5A5 | | | | | |
| 1134 | cg10327400 | LRFN5 | TSS1500 | N_Shore | NA | II | 14 |
| 1135 | cg10330056 | | | | TRUE | II | 2 |
| 1136 | cg10356187 | | | Island | TRUE | II | 12 |
| 1137 | cg10375078 | | | Island | TRUE | I | 10 |
| 1138 | cg10375890 | | | Island | NA | I | 1 |
| 1139 | cg10377903 | | | Island | NA | I | 10 |
| 1140 | cg10383428 | NCAPD2 | 5′UTR | S_Shore | NA | II | 12 |
| 1141 | cg10393598 | | | Island | NA | I | 6 |
| 1142 | cg10397527 | HLA-DOA | Body | S_Shore | NA | I | 6 |
| 1143 | cg10401362 | DNAJB6 | Body | | NA | II | 7 |
| 1144 | cg10417567 | NKX6-1 | Body | Island | TRUE | I | 4 |
| 1145 | cg10435816 | PCDHGA4; PCDHGA | Body; Body; Body; | Island | NA | I | 5 |
| | | 9; PCDHGA1; PCDHG | Body; TSS200; Bod | | | | |
| | | B1; PCDHGB7; PCDH | y; Body; TSS200; Bo | | | | |
| | | GB6; PCDHGB3; PCD | dy; Body; Body; Bod | | | | |
| | | HGB7; PCDHGA6; PC | y; Body; Body; Body | | | | |
| | | DHGA8; PCDHGA10; | ; Body; Body; Body | | | | |
| | | PCDHGA5; PCDHGB4 | | | | | |
| | | ; PCDHGA3; PCDHGA | | | | | |
| | | 2; PCDHGB2; PCDHG | | | | | |
| | | A7; PCDHGB5 | | | | | |
| 1146 | cg10440696 | OR52N1 | 1stExon | | NA | II | 11 |
| 1147 | cg10443049 | NXPHI | Body | N_Shore | NA | II | 7 |
| 1148 | cg10446179 | GJA1 | 5′UTR | | NA | II | 6 |
| 1149 | cg10453019 | GALNTL2; GALNTL2 | 5′UTR; 1stExon | | TRUE | II | 3 |
| 1150 | cg10483275 | CAMK2A; CAMK2A; | 5′UTR; 1stExon; 5′U | | NA | II | 5 |
| | | CAMK2A; CAMK2A | TR; 1stExon | | | | |
| 1151 | cg10499172 | DCPS | TSS1500 | N_Shore | NA | II | 11 |
| 1152 | cg10504392 | | | | TRUE | I | 12 |
| 1153 | cg10512745 | DMIRTA2 | Body | Island | NA | I | 1 |
| 1154 | cg10513702 | NR5A2; NR5A2 | Body; Body | N_Shore | NA | I | 1 |
| 1155 | cg10519368 | PCDHA6; PCDHA2; P | Body; Body; Body; | N_Shore | NA | II | 5 |
| | | CDHA1; PCDHA9; PC | Body; Body; Body; | | | | |
| | | DHA7; PCDHA1; PCD | Body; TSS200; Bod | | | | |
| | | HA6; PCDHA10; PCD | y; Body; Body; TSS2 | | | | |
| | | HA5; PCDHA3; PCDH | 00; TSS200; Body | | | | |
| | | A4; PCDHA10; PCDH | | | | | |
| | | A10; PCDHA8 | | | | | |
| 1156 | cg10552126 | DPP6; DPP6; DPP6 | TSS1500; Body; Bo | Island | TRUE | I | 7 |
| | | | dy | | | | |
| 1157 | cg10558887 | SPG20; SPG20; SPG20; | 5′UTR; 5′UTR; 5′UT | N_Shore | NA | I | 13 |
| | | SPG20 | R; 5′UTR | | | | |
| 1158 | cg10578938 | CYFIP2; CYFIP2; CYF | 5′UTR; TSS1500; 5 | S_Shore | TRUE | II | 5 |
| | | IP2 | UTR | | | | |
| 1159 | cg10583997 | BIRC6 | Body | S_Shore | NA | II | 2 |
| 1160 | cg10593416 | | | Island | NA | II | X |
| 1161 | cg10600889 | FBXO47 | TSS200 | | NA | II | 17 |
| 1162 | cg10603290 | TRIO | Body | | TRUE | II | 5 |
| 1163 | cg10606490 | C11orf88; C11orf88 | TSS200; TSS200 | Island | NA | I | 11 |
| 1164 | cg10613701 | | | N_Shore | NA | II | 8 |
| 1165 | cg10633601 | TAL1 | TSS1500 | Island | TRUE | I | 1 |
| 1166 | cg10658542 | SYT10; SYT10 | 1stExon; 5′UTR | Island | NA | I | 12 |
| 1167 | cg10667338 | LIN7B | TSS1500 | N_Shore | NA | I | 19 |
| 1168 | cg10671180 | SNORD115-39 | TSS200 | | NA | II | 15 |
| 1169 | cg10687131 | GDF7 | Body | Island | NA | II | 2 |
| 1170 | cg10696085 | | | | TRUE | I | 12 |
| 1171 | cg10699857 | | | Island | TRUE | II | 4 |
| 1172 | cg10703826 | TBXI5; TBXI5 | 1stExon; 5′UTR | Island | TRUE | I | 1 |
| 1173 | cg10706039 | | | Island | NA | I | X |
| 1174 | cg10719363 | | | N_Shelf | NA | I | 7 |
| 1175 | cg10745499 | SIMI | Body | Island | NA | II | 6 |
| 1176 | cg10747118 | | | S_Shore | NA | I | 3 |
| 1177 | cg10752158 | | | N_Shelf | NA | II | 4 |
| 1178 | cg10758466 | MPP2 | Body | N_Shelf | NA | II | 17 |
| 1179 | cg10776919 | DMRTA2 | TSS1500 | Island | NA | I | 1 |
| 1180 | cg10784570 | | | Island | NA | II | 11 |
| 1181 | cg10785622 | | | N_Shore | NA | II | 4 |
| 1182 | cg10786329 | | | S_Shore | NA | II | X |
| 1183 | cg10799055 | | | | NA | I | 6 |
| 1184 | cg10801213 | SLC7A3; SLC7A3 | 5′UTR; 5′UTR | Island | NA | II | X |
| 1185 | cg10812708 | | | N_Shore | TRUE | I | 6 |
| 1186 | cg10826197 | | | S_Shore | TRUE | I | 8 |
| 1187 | cg10858215 | | | Island | NA | I | 2 |
| 1188 | cg10862468 | CYP2E1 | Body | Island | NA | II | 10 |
| 1189 | cg10864074 | EPO | TSS1500 | Island | TRUE | I | 7 |
| 1190 | cg10876767 | SORCS2 | Body | | NA | I | 4 |
| 1191 | cg10888031 | ACTRT2 | 1stExon | Island | NA | I | 1 |
| 1192 | cg10893095 | | | | NA | I | 6 |
| 1193 | cg10911913 | SLFN11; SLFN11; SLF | TSS200; TSS200; T | N_Shore | TRUE | II | 17 |
| | | N11; SLFN11; SLFN11 | SS200; TSS200; TS | | | | |
| | | | S200 | | | | |
| 1194 | cg10923838 | CIT | Body | S_Shore | TRUE | II | 12 |
| 1195 | cg10948359 | ME1; ME1 | 5′UTR; 1stExon | Island | NA | I | 6 |
| 1196 | cg10950028 | GSTM1; GSTM1 | Body; Body | S_Shore | NA | I | 1 |
| 1197 | cg10959711 | S100A6 | TSS1500 | S_Shore | NA | I | 1 |
| 1198 | cg11029716 | | | | TRUE | II | 14 |
| 1199 | cg11035122 | MIR758; MIR329-2; | TSS1500; TSS1500 | | NA | II | 14 |
| | | M1I329-1; MIR323 | ; TSS1500; TSS200 | | | | |
| 1200 | cg11044163 | CPVL; CPVL | TSS200; TSS200 | | TRUE | II | 7 |
| 1201 | cg11062466 | | | N_Shore | NA | I | 8 |
| 1202 | cg11062848 | NOL7 | Body | S_Shore | NA | II | 6 |
| 1203 | cg11071926 | NHLH2; NHLH2 | 3′UTR; 3′UTR | N_Shore | NA | II | 1 |
| 1204 | cg11120913 | C3orf14 | TSS1500 | N_Shore | NA | II | 3 |
| 1205 | cg11143193 | | | N_Shelf | NA | II | 12 |
| 1206 | cg11144753 | | | Island | TRUE | I | 14 |
| 1207 | cg11145160 | CLDN11 | TSS1500 | Island | NA | I | 3 |
| 1208 | cg11151395 | MPO | Body | Island | NA | I | 17 |
| 1209 | cg11166197 | GABRA3; MIR105-1 | 5′UTR; TSS1500 | | NA | II | X |
| 1210 | cg11171221 | | | S_Shore | NA | I | 10 |
| 1211 | cg11194545 | NRK; NRK | 5′UTR; 1stExon | Island | NA | I | X |
| 1212 | cg11195360 | OR8B8 | TSS1500 | | NA | II | 11 |
| 1213 | cg11207307 | SLC2A14 | TSS200 | Island | NA | I | 12 |
| 1214 | cg11220565 | | | N_Shore | TRUE | I | 20 |
| 1215 | cg11229513 | PRDM14 | TSS1500 | Island | NA | I | 8 |
| 1216 | cg11231249 | CTNNA2; LARTM1; C | Body; TSS200; Bod | Island | NA | II | 2 |
| | | TNNA2 | y | | | | |
| 1217 | cg11237792 | TTC30A | TSS1500 | S_Shore | NA | II | 2 |
| 1218 | cg11241627 | FERD3L | TSS200 | S_Shore | TRUE | II | 7 |
| 1219 | cg11258089 | PDE4D; PDE4D | Body; TSS200 | Island | TRUE | I | 5 |
| 1220 | cg11268834 | MRPL44 | Body | S_Shore | NA | II | 2 |
| 1221 | cg11269103 | ZNF215 | Body | | NA | II | 11 |
| 1222 | cg11274314 | | | Island | NA | I | 2 |
| 1223 | cg11284797 | | | Island | NA | I | 11 |
| 1224 | cg11309496 | SLFN11; SLFN11; SLF; | TSS1500; TSS1500 | Island | TRUE | I | 17 |
| | | N11; SLFN11; SLFN11 | TSS1500; TSS150 | | | | |
| | | | 10; TSS1500 | | | | |
| 1225 | cg11312408 | PCDH17 | 1stExon | Island | NA | II | 13 |
| 1226 | cg11323198 | CDH8 | 5′UTR | Island | NA | II | 16 |
| 1227 | cg11337289 | DZIP1; DZ1P1 | Body; Body | Island | TRUE | II | 13 |
| 1228 | cg11337915 | HIMGN1 | Body | N_Shore | NA | II | 21 |
| 1229 | cg11347582 | | | Island | NA | I | 6 |
| 1230 | cg11367159 | | | | TRUE | II | 12 |
| 1231 | cg11382963 | MSC | 3′UTR | Island | NA | I | 8 |
| 1232 | cg11386025 | KCNQ4; KCNQ4 | Body; Body | Island | TRUE | I | 1 |
| 1233 | cg11389889 | NOP56; NOP56; SNOR | Body; Body; Body; T | S_Shore | NA | II | 20 |
| | | D110; SNORA51 | SS 1500 | | | | |
| 1234 | cg11401794 | NKD2 | Body | | NA | I | 5 |
| 1235 | cg11409022 | ST3GAL3; ST3GAL3; | Body; Body; Body; | | TRUE | II | 1 |
| | | ST3GAL3; ST3GAL3; | Body; Body; Body; | | | | |
| | | ST3GAL3; ST3GAL3; | Body; Body; Body; | | | | |
| | | ST3GAL3; ST3GAL3; | Body | | | | |
| | | ST3GAL3; ST3GAL3 | | | | | |
| 1236 | cg11410682 | PNMAL2 | TSS200 | Island | NA | I | 19 |
| 1237 | cg11412129 | | | S_Shore | NA | I | 1 |
| 1238 | cg11412853 | | | N_Shore | TRUE | II | 12 |
| 1239 | cg11416384 | KIAA1239 | 1stExon | Island | NA | I | 4 |
| 1240 | cg11431957 | VILL | Body | Island | NA | II | 3 |
| 1241 | cg11432962 | | | Island | TRUE | II | 13 |
| 1242 | cg11433319 | CHODL | Body | Island | NA | I | 21 |
| 1243 | cg11435506 | PON3 | TSS1500 | Island | NA | I | 7 |
| 1244 | cg11439596 | | | Island | NA | I | 5 |
| 1245 | cg11452329 | | | | NA | I | 6 |
| 1246 | cg11458217 | LOC644538; LOC6445 | 1stExon; 5′UTR | Island | TRUE | I | X |
| | | 38 | | | | | |
| 1247 | cg11459603 | HIST1H2BI | 1stExon | Island | NA | II | 6 |
| 1248 | cg11460714 | | | | NA | II | 7 |
| 1249 | cg11471772 | SIM1 | TSS200 | N_Shore | TRUE | I | 6 |
| 1250 | cg11485152 | TRNT1 | Body | S_Shelf | NA | II | 3 |
| 1251 | cg11485595 | NPR3 | Body | Island | NA | II | 5 |
| 1252 | cg11497372 | | | | NA | I | 6 |
| 1253 | cg11503104 | C1orf86; LOC1001280 | Body; TSS200; Bod | N_Shore | NA | II | 1 |
| | | 03; C1orf86 | y | | | | |
| 1254 | cg11504718 | ATP11A; ATP11A | 3′UTR; 3′UTR | S_Shore | NA | II | 13 |
| 1255 | cg11521404 | GJA3 | TSS1500 | Island | NA | I | 13 |
| 1256 | cg11521965 | PTN | TSS200 | | NA | II | 7 |
| 1257 | cg11525280 | C2orf39 | TSS200 | Island | TRUE | I | 2 |
| 1258 | cg11550547 | KCTD14 | TSS200 | | NA | II | 11 |
| 1259 | cg11564981 | NRN1 | TSS200 | Island | TRUE | I | 6 |
| 1260 | cg11567288 | | | | NA | II | 6 |
| 1261 | cg11574975 | ADORA3 | Body | Island | TRUE | I | 1 |
| 1262 | cg11580212 | | | | TRUE | II | 5 |
| 1263 | cg11580948 | FGF5; FGF5 | Body; Body | S_Shore | NA | II | 4 |
| 1264 | cg11593123 | PCDH11X | 5′UTR | Island | NA | II | X |
| 1265 | cg11595135 | | | Island | NA | II | X |
| 1266 | cg11601252 | LBXCOR1 | Body | Island | NA | I | 15 |
| 1267 | cg11605903 | | | S_Shelf | NA | II | 6 |
| 1268 | cg11616651 | AMH | Body | Island | NA | I | 19 |
| 1269 | cg11640591 | PTH1R | Body | Island | NA | II | 3 |
| 1270 | cg11641791 | KRT222 | TSS200 | | NA | I | 17 |
| 1271 | cg11642106 | | | S_Shore | TRUE | I | 3 |
| 1272 | cg11645465 | | | | NA | II | 8 |
| 1273 | cg11648594 | MMP23A; MMP23B | TSS1500; TSS1500 | Island | TRUE | I | 1 |
| 1274 | cg11671688 | GPR6 | 1stExon | Island | NA | I | 6 |
| 1275 | cg11678027 | C13orf38; C13orf38; C1 | TSS200; TSS200; T | Island | NA | I | 13 |
| | | 3orf38; C13orf38; C13o | SS200; TSS200; TS | | | | |
| | | r138; C13orf38 | S200; TSS200 | | | | |
| 1276 | cg11680055 | GSTM1; GSTM1 | TSS200; TSS200 | Island | NA | I | 1 |
| 1277 | cg11684647 | LRRK2 | TSS1500 | Island | NA | II | 12 |
| 1278 | cg11687036 | | | Island | TRUE | II | 3 |
| 1279 | cg11690724 | EBF1 | Body | N_Shelf | NA | II | 5 |
| 1280 | cg11690979 | C1orf230 | Body | S_Shore | NA | II | 1 |
| 1281 | cg11708358 | LRP11 | Body | N_Shore | NA | II | 6 |
| 1282 | cg11712990 | LOC645323; LOC6453 | Body; Body; Body | N_Shore | NA | I | 5 |
| | | 23; LOC645323 | | | | | |
| 1283 | cg11720950 | | | | TRUE | II | 6 |
| 1284 | cg11731890 | HIST1H3G; HIST1H2 | TSS1500; TSS1500 | N_Shore | NA | II | 6 |
| | | BI | | | | | |
| 1285 | cg11735997 | KCNA4 | TSS1500 | Island | NA | II | 11 |
| 1286 | cg11737164 | VGLL3 | Body | N_Shore | NA | II | 3 |
| 1287 | cg11739675 | CHST11 | Body | Island | NA | I | 12 |
| 1288 | cg11748187 | TCF7L2; TCF7L2; TCF | Body; Body; Body; | S_Shore | NA | II | 10 |
| | | 7L2; TCF7L2; TCF7L2; | Body; Body; Body | | | | |
| | | TCF7L2 | | | | | |
| 1289 | cg11756095 | | | Island | TRUE | I | 1 |
| 1290 | cg11791751 | PDPN; PDPN | TSS200; TSS200 | Island | NA | II | 1 |
| 1291 | cg11805669 | | | S_Shore | NA | I | 2 |
| 1292 | cg11811513 | ZNF804A | TSS1500 | N_Shore | NA | II | 2 |
| 1293 | cg11832210 | SLC16A12 | TSS200 | Island | TRUE | I | 10 |
| 1294 | cg11833001 | | | Island | NA | II | 3 |
| 1295 | cg11840125 | EFS; EFS | TSS200; TSS200 | Island | NA | I | 14 |
| 1296 | cg11856078 | | | S_Shore | NA | I | 2 |
| 1297 | cg11856897 | | | Island | TRUE | I | 1 |
| 1298 | cg11857704 | | | Island | NA | I | 2 |
| 1299 | cg11859529 | SLC7A3; SLC7A3; SL | 1stExon; 5′UTR; 1st | Island | NA | I | X |
| | | C7A3; SLC7A3 | Exon; 5′UTR | | | | |
| 1300 | cg11861562 | TAGLN; TAGLN | TSS1500; TSS1500 | | NA | I | 11 |
| 1301 | cg11874123 | PTN; PTN | 5′UTR; 1stExon | | NA | II | 7 |
| 1302 | cg11889692 | | | Island | TRUE | I | 14 |
| 1303 | cg11891925 | | | | NA | II | 10 |
| 1304 | cg11898009 | | | Island | TRUE | I | 7 |
| 1305 | cg11898486 | LYPD5; LYPD5 | 5′UTR; 1stExon | Island | NA | I | 19 |
| 1306 | cg11900861 | FLJ22536 | TSS200 | Island | NA | II | 6 |
| 1307 | cg11901043 | | | Island | NA | I | 12 |
| 1308 | cg11935153 | | | Island | TRUE | II | 6 |
| 1309 | cg11947981 | ITGA4 | Body | Island | NA | II | 2 |
| 1310 | cg11953383 | ME3; ME3; ME3 | 5′UTR; TSS200; 5′U | Island | NA | I | 11 |
| | | | TR | | | | |
| 1311 | cg11973479 | | | Island | NA | II | 7 |
| 1312 | cg11980129 | ROBO1 | 5′UTR | Island | NA | II | 3 |
| 1313 | cg11982736 | CRIP3 | TSS200 | Island | TRUE | I | 6 |
| 1314 | cg12005098 | SLC16A12 | TSS200 | Island | TRUE | I | 10 |
| 1315 | cg12011198 | HDAC4 | 3′UTR | Island | NA | I | 2 |
| 1316 | cg12014547 | NELL1; NELL1 | Body; Body | S_Shore | NA | I | 11 |
| 1317 | cg12016746 | LRP5 | Body | S_Shelf | NA | II | 11 |
| 1318 | cg12019614 | DOCK6 | Body | Island | TRUE | I | 19 |
| 1319 | cg12029804 | KRT222; KRT222 | 5′UTR; 1stExon | | NA | II | 17 |
| 1320 | cg12030550 | ME3; ME3; ME3 | TSS1500; TSS200; | S_Shore | NA | II | 11 |
| | | | TSS1500 | | | | |
| 1321 | cg12094871 | ATXN3; ATXN3; ATX | Body; 3′UTR; Body; | | TRUE | II | 14 |
| | | N3; ATXN3; ATXN3; A | Body; Body; 3′UTR; | | | | |
| | | TXN3; ATXN3; ATXN | Body; Body; 3′UTR; | | | | |
| | | 3; ATXN3; ATXN3; AT | Body; 3′UTR; 3′UT | | | | |
| | | XN3; ATXN3; ATXN3; | R; Body; Body; 3′UT | | | | |
| | | ATXN3; ATXN3; ATX | R; Body; Body; B0d | | | | |
| | | N3; ATXN3; ATXN3; A | y; 3′UTR; Body; 3′U | | | | |
| | | TXN3; ATXN3; ATXN | TR; 3′UTR; Body; B | | | | |
| | | 3; ATXN3; ATXN3; AT | ody; Body; 3′UTR; 3′ | | | | |
| | | XN3; ATXN3; ATXN3; | UTR; 3 ′UTR; Body; | | | | |
| | | ATXN3; ATXN3; ATX | Body; Body; 3′UTR | | | | |
| | | N3; ATXN3; ATXN3; A | | | | | |
| | | TXN3 | | | | | |
| 1322 | cg12052765 | SLC18A3; CHAT | TSS1500; TSS200 | N_Shore | NA | II | 10 |
| 1323 | cg12090430 | ADARB2 | Body | | NA | II | 10 |
| 1324 | cg12093519 | | | N_Shore | TRUE | II | 7 |
| 1325 | cg12103152 | HTR1B | TSS200 | Island | NA | II | 6 |
| 1326 | cg12106728 | | | Island | NA | I | 12 |
| 1298 | cg11857704 | | | Island | NA | I | 2 |
| 1299 | cg11859529 | SLC7A3; SLC7A3; SL | 1stExon; 5′UTR; 1st | | | | |
| | | C7A3; SLC7A3 | Exon; 5′UTR | Island | NA | I | X |
| 1300 | cg11861562 | TAGLN; TAGLN | TSS1500; TSS1500 | | NA | I | 11 |
| 1301 | cg11874123 | PTN; PTN | 5′UTR; 1stExon | | NA | II | 7 |
| 1302 | cg11889692 | | | Island | TRUE | I | 14 |
| 1303 | cg11891925 | | | | NA | II | 10 |
| 1304 | cg11898009 | | | Island | TRUE | I | 7 |
| 1305 | cg11898486 | LYPD5; LYPD5 | 5′UTR; 1stExon | Island | NA | I | 19 |
| 1306 | cg11900861 | FLJ22536 | TSS200 | Island | NA | II | 6 |
| 1307 | cg11901043 | | | Island | NA | I | 12 |
| 1308 | cg11935153 | | | Island | TRUE | II | 6 |
| 1309 | cg11947981 | ITGA4 | Body | Island | NA | II | 2 |
| 1310 | cg11953383 | ME3; ME3; ME3 | 5′UTR; TSS200; 5′U | Island | NA | I | 11 |
| | | | TR | | | | |
| 1311 | cg11973479 | | | Island | NA | II | 7 |
| 1312 | cg11980129 | ROB01 | 5′UTR | Island | NA | II | 3 |
| 1313 | cg11982736 | CR1P3 | TSS200 | Island | TRUE | I | 6 |
| 1314 | cg12005098 | SLC16A12 | TSS200 | Island | TRUE | I | 10 |
| 1315 | cg12011198 | HIDAC4 | 3′UTR | Island | NA | I | 2 |
| 1316 | cg12014547 | NELL1; NELL1 | Body; Body | S_Shore | NA | I | 11 |
| 1317 | cg12016746 | LRP5 | Body | S_Shelf | NA | II | 11 |
| 1318 | cg12019614 | DOCK6 | Body | Island | TRUE | I | 19 |
| 1319 | cg12029804 | KRT222; KRT222 | 5′UTR; 1stExon | | NA | II | 17 |
| 1320 | cg12030550 | ME3; ME3; ME3 | TSS1500; TSS200; | S_Shore | NA | II | 11 |
| | | | TSS1500 | | | | |
| 1321 | cg12034871 | ATXN3; ATXN3; ATX | Body; 3′UTR; Body; | | TRUE | II | 14 |
| | | N3; ATXN3; ATXN3; A | Body; Body; 3′UTR; | | | | |
| | | TXN3; ATXN3; ATXN | Body; Body; 3′UTR; | | | | |
| | | 3; ATXN3; ATXN3; AT | Body; 3′UTR; 3′UT | | | | |
| | | XN3; ATXN3; ATXN3; | R; Body; Body; 3′UT | | | | |
| | | ATXN3; ATXN3; ATX | R; Body; Body; Bod | | | | |
| | | N3; ATXN3; ATXN3; A | y; 3′UTR; Body; 3′U | | | | |
| | | TXN3; ATXN3; ATXN | TR; 3′UTR; Body; B | | | | |
| | | 3; ATXN3; ATXN3; AT | ody; Body; 3′UTR; 3′ | | | | |
| | | XN3; ATXN3; ATXN3; | UTR; 3′UTR; Body; | | | | |
| | | ATXN3; ATXN3; ATX | Body; Body; 3′UTR | | | | |
| | | N3; ATXN3; ATXN3; A | | | | | |
| | | TXN3 | | | | | |
| 1322 | cg12052765 | SLC18A3; CHAT | TSS1500; TSS200 | N_Shore | NA | II | 10 |
| 1323 | cg12090430 | ADARB2 | Body | | NA | II | 10 |
| 1324 | cg12093519 | | | N_Shore | TRUE | II | 7 |
| 1325 | cg12103152 | HTR1B | TSS200 | Island | NA | II | 6 |
| 1326 | cg12106728 | | | Island | NA | I | 12 |
| 1298 | cg11857704 | | | Island | NA | I | 2 |
| 1299 | cg11859529 | SLC7A3; SLC7A3; SL | 1stExon; 5′UTR; 1st | Island | NA | I | X |
| | | C7A3; SLC7A3 | Exon; 5′UTR | | | | |
| 1300 | cg11861562 | TAGLN; TAGLN | TSS1500; TSS1500 | | NA | I | 11 |
| 1301 | cg11874123 | PTN; PTN | 5′UTR; 1stExon | | NA | II | 7 |
| 1302 | cg11889692 | | | Island | TRUE | I | 14 |
| 1303 | cg11891925 | | | | NA | II | 10 |
| 1304 | cg11898009 | | | Island | TRUE | I | 7 |
| 1305 | cg11898486 | LYPD5; LYPD5 | 5′UTR; 1stExon | Island | NA | I | 19 |
| 1306 | cg11900861 | FLJ22536 | TSS200 | Island | NA | II | 6 |
| 1307 | cg11901043 | | | Island | NA | I | 12 |
| 1308 | cg11935153 | | | Island | TRUE | II | 6 |
| 1309 | cg11947981 | ITGA4 | Body | Island | NA | II | 2 |
| 1310 | cg11953383 | ME3; ME3; ME3 | 5′UTR; TSS200; 5′UTR | Island | NA | I | 11 |
| 1311 | cg11973479 | | | Island | NA | II | 7 |
| 1312 | cg11980129 | ROBO1 | 5′UTR | Island | NA | II | 3 |
| 1313 | cg11982736 | CRIP3 | TSS200 | Island | TRUE | I | 6 |
| 1314 | cg12005098 | SLC16A12 | TSS200 | Island | TRUE | I | 10 |
| 1315 | cg12011198 | HDAC4 | 3′UTR | Island | NA | | 2 |
| 1316 | cg12014547 | NELL1; NELL1 | Body; Body | S_Shore | NA | I | 11 |
| 1317 | cg12016746 | LRP5 | Body | S_Shelf | NA | II | 11 |
| 1318 | cg12019614 | DOCK6 | Body | Island | TRUE | I | 19 |
| 1319 | cg12029804 | KRT222; KRT222 | 5′UTR; 1stExon | | NA | II | 17 |
| 1320 | cg12030550 | ME3; ME3KAE3 | TSS1500; TSS200; | S_Shore | NA | II | 11 |
| | | | TSS1500 | | | | |
| 1321 | cg12034871 | ATXN3; ATXN3; ATX | Body; 3′UTR; Body; | | TRUE | II | 14 |
| | | N3; ATXN3; ATXN3; A | Body; Body; 3′UTR; | | | | |
| | | TXN3; ATXN3; ATXN | Body; Body; 3′UTR; | | | | |
| | | 3; ATXN3; ATXN3; AT | Body; 3′UTR; 3′UT | | | | |
| | | XN3; ATXN3; ATXN3; | R; Body; Body; 3′UT | | | | |
| | | ATXN3; ATXN3; ATX | R; Body; Body; Bod | | | | |
| | | N3; ATXN3; ATXN3; A | y; 3′UTR; Body; 3′U | | | | |
| | | TXN3; ATXN3; ATXN | TR; 3′UTR; Body; B | | | | |
| | | 3; ATXN3; ATXN3; AT | ody; Body; 3′UTR; 3′ | | | | |
| | | XN3; ATXN3; ATXN3; | UTR; 3′UTR; Body; | | | | |
| | | ATXN3; ATXN3; ATX | Body; Body; 3′UTR | | | | |
| | | N3; ATXN3; ATXN3; A | | | | | |
| | | TXN3 | | | | | |
| 1322 | cg12052765 | SLC18A3; CHAT | TSS1500; TSS200 | N_Shore | NA | II | 10 |
| 1323 | cg12090430 | ADARB2 | Body | | NA | II | 10 |
| 1324 | cg12093519 | | | N_Shore | TRUE | II | 7 |
| 1325 | cg12103152 | HTR1B | TSS200 | Island | NA | II | 6 |
| 1326 | cg12106728 | | | Island | NA | I | 12 |
| 1298 | cg11857704 | | | Island | NA | I | 2 |
| 1299 | cg11859529 | SLC7A3; 5LC7A3; SL | 1stExon; 5′UTR; 1st | Island | NA | I | X |
| | | C7A3; SLC7A3 | Exon; 5′UTR | | | | |
| 1300 | cg11861562 | TAGLN; TAGLN | TSS1500; TSS1500 | | NA | I | 11 |
| 1301 | cg11874123 | PTN; PTN | 5 ′UTR; 1stExon | | NA | II | 7 |
| 1302 | cg11889692 | | | Island | TRUE | I | 14 |
| 1303 | cg11891925 | | | | NA | II | 10 |
| 1304 | cg11898009 | | | Island | TRUE | I | 7 |
| 1305 | cg11898486 | LYPD5; LYPD5 | 5′UTR; 1stExon | Island | NA | I | 19 |
| 1306 | cg11900861 | FLJ22536 | TSS200 | Island | NA | II | 6 |
| 1307 | cg11901043 | | | Island | NA | I | 12 |
| 1308 | cg11935153 | | | Island | TRUE | II | 6 |
| 1309 | cg11947981 | ITGA4 | Body | Island | NA | II | 2 |
| 1310 | cg11953383 | ME3; ME3; ME3 | 5′UTR; TSS200; 5′U | Island | NA | I | 11 |
| | | | TR | | | | |
| 1311 | cg11973479 | | | Island | NA | II | 7 |
| 1312 | cg11980129 | ROBO1 | 5′UTR | Island | NA | II | 3 |
| 1313 | cg11982736 | CRIP3 | TSS200 | Island | TRUE | I | 6 |
| 1314 | cg12005098 | SLC16A12 | TSS200 | Island | TRUE | I | 10 |
| 1315 | cg12011198 | HDAC4 | 3′UTR | Island | NA | I | 2 |
| 1316 | cg12014547 | NELL1; NELL1 | Body; Body | S_Shore | NA | I | 11 |
| 1317 | cg12016746 | LRP5 | Body | S_Shelf | NA | II | 11 |
| 1318 | cg12019614 | DOCK6 | Body | Island | TRUE | I | 19 |
| 1319 | cg12029804 | KRT222; KRT222 | 5′UTR; 1stExon | | NA | II | 17 |
| | | | TSS1500 | | | | |
| TSS1 | S_Shore | NA | II | 11 | 86383713 | GGGGATTT | |
| 500 | | | | | | GCGTTTGG | |
| | | | | | | GGGTCAGC | |
| | | | | | | ATATCCTC | |
| | | | | | | GCACTCTC | |
| | | | | | | CCAACATT | |
| | | | | | | CG | |
| 1321 | cg12034871 | ATXN3; ATXN3; ATX | Body; 3′UTR; Body; | | TRUE | II | 14 |
| | | N3; ATXN3; ATXN3; A | Body; Body; 3′UTR; | | | | |
| | | TXN3; ATXN3; ATXN | Body; Body; 3′UTR; | | | | |
| | | 3; ATXN3; ATXN3; AT | Body; 3′UTR; 3′UT | | | | |
| | | XN3; ATXN3; ATXN3; | R; Body; Body; 3′UT | | | | |
| | | ATXN3; ATXN3; ATX | R; Body; Body; Bod | | | | |
| | | N3; ATXN3; ATXN3; A | y; 3′UTR; Body; 3′U | | | | |
| | | TXN3; ATXN3; ATXN | TR; 3′UTR; Body; B | | | | |
| | | 3; ATXN3; ATXN3; AT | ody; Body; 3′UTR; 3′ | | | | |
| | | XN3; ATXN3; ATXN3; | UTR; 3′UTR; Body; | | | | |
| | | ATXN3; ATXN3; ATX | Body; Body; 3′UTR | | | | |
| | | N3; ATXN3; ATXN3; A | | | | | |
| | | TXN3 | | | | | |
| 1322 | cg12052765 | SLC18A3; CHAT | TSS1500; TSS200 | N_Shore | NA | II | 10 |
| 1323 | cg12090430 | ADARB2 | Body | | NA | II | 10 |
| 1324 | cg12093519 | | | N_Shore | TRUE | II | 7 |
| 1325 | cg12103152 | HTR1B | TSS200 | Island | NA | II | 6 |
| 1326 | cg12106728 | | | Island | NA | I | 12 |
| 1298 | cg11857704 | | | Island | NA | I | 2 |
| 1299 | cg11859529 | SLC7A3; SLC7A3; SL | 1stExon; 5′UTR; 1st | Island | NA | I | X |
| | | C7A3; SLC7A3 | Exon; 5′UTR | | | | |
| 1300 | cg11861562 | TAGLN; TAGLN | TSS1500; TSS1500 | | NA | I | 11 |
| 1301 | cg11874123 | PTN PTN | 5′UTR; 1stExon | | NA | II | 7 |
| 1302 | cg11889692 | | | Island | TRUE | I | 14 |
| 1303 | cg11891925 | | | | NA | II | 10 |
| 1304 | cg11898009 | | | Island | TRUE | I | 7 |
| 1305 | cg11898486 | LYPD5; LYPD5 | 5′UTR; 1stExon | Island | NA | I | 19 |
| 1306 | cg11900861 | FLJ22536 | TSS200 | Island | NA | II | 6 |
| 1307 | cg11901043 | | | Island | NA | I | 12 |
| 1308 | cg11935153 | | | Island | TRUE | II | 6 |
| 1309 | cg11947981 | ITGA4 | Body | Island | NA | II | 2 |
| 1310 | cg11953383 | ME3; ME3; ME3 | 5′UTR; TSS200; 5′U | Island | NA | I | 11 |
| | | | TR | | | | |
| 1311 | cg11973479 | | | Island | NA | II | 7 |
| 1312 | cg11980129 | ROBO1 | 5′UTR | Island | NA | II | 3 |
| 1313 | cg11982736 | CRIP3 | TSS200 | Island | TRUE | I | 6 |
| 1314 | cg12005098 | SLC16A12 | TSS200 | Island | TRUE | I | 10 |
| 1315 | cg12011198 | HDAC4 | 3′UTR | Island | NA | I | 2 |
| 1316 | cg12014547 | NELL1; NELL1 | Body; Body | S_Shore | NA | I | 11 |
| 1317 | cg12016746 | LRP5 | Body | S_Shelf | NA | II | 11 |
| 1318 | cg12019614 | DOCK6 | Body | Island | TRUE | I | 19 |
| 1319 | cg12029804 | KRT222; KRT222 | 5′UTR; 1stExon | | NA | II | 17 |
| 1320 | cg12030550 | ME3; ME3; ME3 | TSS1500; TSS200; | S_Shore | NA | II | 11 |
| | | | TSS1500 | | | | |
| 1321 | cg12034871 | ATXN3; ATXN3; ATX | Body; 3′UTR; Body; | | TRUE | II | 14 |
| | | N3; ATXN3; ATXN3; A | Body; Body; 3′UTR; | | | | |
| | | TXN3; ATXN3; ATXN | Body; Body; 3′UTR; | | | | |
| | | 3; ATXN3; ATXN3; AT | Body; 3′UTR; 3′UT | | | | |
| | | XN3; ATXN3; ATXN3; | R; Body; Body; 3′UT | | | | |
| | | ATXN3; ATXN3; ATX | R; Body; Body; Bod | | | | |
| | | N3; ATXN3; ATXN3; A | y; 3′UTR; Body; 3′U | | | | |
| | | TXN3; ATXN3; ATXN | TR; 3′UTR; Body; B | | | | |
| | | 3; ATXN3; ATXN3; AT | ody; Body; 3′UTR; 3′ | | | | |
| | | XN3; ATXN3; ATXN3; | UTR; 3′UTR; Body; | | | | |
| | | ATXN3; ATXN3; ATX | Body; Body; 3′UTR | | | | |
| | | N3; ATXN3; ATXN3; A | | | | | |
| | | TXN3 | | | | | |
| 1322 | cg12052765 | SLC18A3; CHAT | TSS1500; TSS200 | N_Shore | NA | II | 10 |
| 1323 | cg12090430 | ADARB2 | Body | | NA | II | 10 |
| 1324 | cg12093519 | | | N_Shore | TRUE | II | 7 |
| 1325 | cg12103152 | HTR1B | TSS200 | Island | NA | II | 6 |
| 1326 | cg12106728 | | | Island | NA | I | 12 |
| 1327 | cg12113984 | | | Island | TRUE | II | 12 |
| 1328 | cg12116395 | ATP6AP2 | Body | S_Shore | NA | I | X |
| 1329 | cg12126857 | | | | TRUE | I | 1 |
| 1330 | cg12135344 | CHD5 | Body | Island | TRUE | I | 1 |
| 1331 | cg12164612 | KCNA4 | 5′UTR | Island | NA | I | 11 |
| 1332 | cg12166610 | NRG1 | Body | Island | NA | I | 8 |
| 1333 | cg12169365 | RIN3 | TSS1500 | N_Shore | NA | I | 14 |
| 1334 | cg12170314 | INSC; INSC; INSC | Body; 5′UTR; 1stEx | Island | NA | I | 11 |
| | | | on | | | | |
| 1335 | cg12173409 | | | Island | NA | II | 3 |
| 1336 | cg12177793 | NFATC4; NFATC4 | Body; Body | Island | NA | I | 14 |
| 1337 | cg12193731 | ACVRL1 | TSS1500 | Island | NA | I | 12 |
| 1338 | cg12196692 | HEATR7B2; HEATR7 | 1stExon; 5′UTR | | NA | II | 5 |
| | | B2 | | | | | |
| 1339 | cg12217600 | | | | NA | I | 10 |
| 1340 | cg12254845 | | | Island | TRUE | II | 15 |
| 1341 | cg12268236 | PRDM16; PRDM16 | Body; Body | Island | NA | I | 1 |
| 1342 | cg12287936 | NDUFS6; MRPL36 | TSS1500; TSS1500 | Island | NA | II | 5 |
| 1343 | cg12295100 | GPRASP1; GPRASP1; | 5′UTR; 5′UTR; 5′UT | Island | NA | I | X |
| | | GPRASP1 | R | | | | |
| 1344 | cg12302982 | | | Island | NA | I | 2 |
| 1345 | cg12311175 | NDST3 | Body | | NA | II | 4 |
| 1346 | cg12323723 | | | Island | NA | I | X |
| 1347 | cg12337525 | | | N_Shelf | NA | II | 4 |
| 1348 | cg12341047 | FGF5; FGF5 | Body; Body | S_Shore | NA | II | 4 |
| 1349 | cg12347429 | FLJ22536 | Body | Island | NA | I | 6 |
| 1350 | cg12367389 | HTR2A; HTR2A | Body; Body | | TRUE | I | 13 |
| 1351 | cg12368188 | | | Island | NA | I | 15 |
| 1352 | cg12396306 | PCDHB14 | TSS1500 | N_Shore | NA | I | 5 |
| 1353 | cg12403253 | ABT1 | Body | N_Shore | NA | II | 6 |
| 1354 | cg12421282 | NFYC; NFYC; LOC100 | 5′UTR; 5′UTR; TSS | S_Shore | NA | II | 1 |
| | | 130557; NFYC; NFYC; | 1500; 5′UTR; 5′UTR | | | | |
| | | NFYC | ; 5′UTR | | | | |
| 1355 | cg12444328 | LHX9; LHX9 | Body; Body | S_Shore | NA | II | 1 |
| 1356 | cg12452298 | | | N_Shore | TRUE | I | 15 |
| 1357 | cg12463578 | ZFP57 | 1stExon | | NA | I | 6 |
| 1358 | cg12467264 | | | N_Shore | TRUE | I | 5 |
| 1359 | cg12473912 | | | Island | NA | I | 3 |
| 1360 | cg12489727 | TNRC6C; TNRC6C | 3′UTR; 3′UTR | S_Shore | NA | II | 17 |
| 1361 | cg12499092 | NFYB | 3′UTR | | NA | II | 12 |
| 1362 | cg12499235 | ASCL2 | TSS1500 | S_Shore | NA | I | 11 |
| 1363 | cg12506775 | C10orf18 | 5′UTR | | TRUE | II | 10 |
| 1364 | cg12513880 | | | Island | NA | I | 12 |
| 1365 | cg12534150 | APC; APC; APC; APC; | 1stExon; 5′UTR; 1st | | TRUE | I | 5 |
| | | APC | Exon; 5′UTR; 5′UT | | | | |
| | | | R | | | | |
| 1366 | cg12563839 | C14orf159; C14orf159; | 1stExon; 1stExon; 5′ | | NA | II | 14 |
| | | C14orf159 C14orf159; | UTR; 1stExon; 5′UT | | | | |
| | | C14orf159; C14orf159; | R; 5′UTR; TSS1500; | | | | |
| | | C14orf159; C14orf159; | 1stExon; 5′UTR | | | | |
| | | C14orf159 | | | | | |
| 1367 | cg12584111 | MOXD2 | Body | | NA | I | 7 |
| 1368 | cg12584785 | CHRNA7 | Body | S_Shelf | NA | II | 15 |
| 1369 | cg12598089 | KCNAB1; KCNAB1; K | TSS200; Body; Bod | N_Shore | TRUE | II | 3 |
| | | CNAB1 | y | | | | |
| 1370 | cg12610744 | KRT4 | TSS1500 | | NA | II | 12 |
| 1371 | cg12610917 | IRF2BP1 | 1stExon | Island | TRUE | II | 19 |
| 1372 | cg12620035 | | | S_Shore | NA | I | 7 |
| 1373 | cg12634080 | | | Island | NA | I | 13 |
| 1374 | cg12642693 | SHROOM4; SHROOM | 1stExon; Body | Island | NA | II | X |
| | | 4 | | | | | |
| 1375 | cg12698813 | | | Island | NA | II | 12 |
| 1376 | cg12727795 | PDGFRB | TSS1500 | | NA | II | 5 |
| 1377 | cg12728791 | HMCN1 | Body | | NA | II | 1 |
| 1378 | cg12741994 | CLDN11 | Body | Island | TRUE | II | 3 |
| 1379 | cg12744758 | | | | NA | I | 13 |
| 1380 | cg12744820 | OLIG3 | 1stExon | Island | TRUE | I | 6 |
| 1381 | cg12754571 | LOC148824; OR2C3 | TSS200; 3′UTR | Island | NA | I | 1 |
| 1382 | cg12768605 | LYPD5 | TSS200 | Island | NA | II | 19 |
| 1383 | cg12784272 | | | | TRUE | II | 17 |
| 1384 | cg12787405 | | | Island | NA | II | 20 |
| 1385 | cg12799885 | | | Island | NA | I | 10 |
| 1386 | cg12814550 | | | S_Shore | NA | I | X |
| 1387 | cg12819011 | C12orf35 | Body | | TRUE | II | 12 |
| 1388 | cg12827262 | TULP1 | Body | Island | NA | II | 6 |
| 1389 | cg12829717 | DDIT4L | 5′UTR | Island | NA | I | 4 |
| 1390 | cg12832313 | PCDHAC2; PCDHAC2 | 1stExon; TSS200; B | Island | NA | I | 5 |
| | | ; PCDHA7; PCDHAl2; | ody; Body; Body; Bo | | | | |
| | | PCDHA6; PCDHA10; P | dy; Body; Body; Bod | | | | |
| | | CDHA4; PCDHA11; P | y; Body; Body; Body | | | | |
| | | CDHA8; PCDHA6; PC | ; Body; Body; Body; | | | | |
| | | DHA1; PCDHA2; PCD | Body; Body; Body; 5 | | | | |
| | | HA1; PCDHA9; PCDH | ′UTR; Body | | | | |
| | | A 13; PCDHA5; PCDH | | | | | |
| | | AC1; PCDHA3; PCDH | | | | | |
| | | AC2; PCDHA10 | | | | | |
| 1391 | cg12833465 | SFTA3 | Body | Island | TRUE | II | 14 |
| 1392 | cg12836959 | | | | NA | I | 3 |
| 1393 | cg12847554 | SIM1 | Body | Island | NA | II | 6 |
| 1394 | cg12850379 | TCEA1; TCEA1 | TSS1500; TSS1500 | S_Shore | NA | I | 8 |
| 1395 | cg12857541 | DHX15 | Body | N_Shelf | NA | II | 4 |
| 1396 | cg12876356 | GFI1; GFI1; GFI1 | Body; Body; Body | Island | TRUE | II | 1 |
| 1398 | cg12883279 | PPT2; PRRT1; PPT2 | TSS1500; TSS1500 | N_Shore | TRUE | I | 6 |
| | | | ; TSS1500 | | | | |
| 1399 | cg12898512 | TSTD1; TSTD1; TSTD | Body; 3′UTR; Body | N_Shore | NA | II | 1 |
| | | 1 | | | | | |
| 1400 | cg12922751 | | | N_Shore | TRUE | II | 14 |
| 1401 | cg12938068 | | | N_Shore | NA | I | 13 |
| 1402 | cg12940439 | | | Island | NA | II | 1 |
| 1403 | cg12950441 | HTR2C | TSS200 | Island | TRUE | I | X |
| 1404 | cg12979234 | DBNL; DBNL; DBNL | TSS1500; TSS1500 | N_Shore | NA | II | 7 |
| | | | ; TSS1500 | | | | |
| 1405 | cg13000082 | AOX1 | TSS1500 | Island | TRUE | II | 2 |
| 1406 | cg13004182 | | | Island | TRUE | I | 5 |
| 1407 | cg13012115 | OR8B2 | TSS1500 | | NA | II | 11 |
| 1408 | cg13033070 | | | Island | NA | I | 5 |
| 1409 | cg13038560 | C2orf60; C2orf60; C2or | Body; Body; TSS15 | N_Shore | NA | II | 2 |
| | | f47 | 00 | | | | |
| 1410 | cg13046832 | C14orf23 | Body | Island | NA | I | 14 |
| 1411 | cg13060157 | | | Island | TRUE | I | 1 |
| 1412 | cg13062913 | NCAM2 | TSS1500 | Island | NA | II | 21 |
| 1413 | cg13097407 | CRIP3 | TSS200 | Island | TRUE | II | 6 |
| 1414 | cg13101087 | GALNT13 | 5′UTR | Island | NA | I | 2 |
| 1415 | cg13121699 | ZNF804A | TSS1500 | N_Shore | NA | II | 2 |
| 1416 | cg13164157 | PROM1; PROM1 | 5′UTR; 5′UTR | Island | TRUE | II | 4 |
| 1417 | cg13170005 | VMO1; VMO1; VMO1; | TSS1500; TSS1500 | N_Shore | NA | II | 17 |
| | | VMO1 | ; TSS1500; TSS150 | | | | |
| | | | 0 | | | | |
| 1418 | cg13174253 | C8orf33 | Body | S_Shore | NA | II | 8 |
| 1419 | cg13180315 | | | N_Shore | NA | I | 2 |
| 1420 | cg13181164 | MEIS2; MEIS2; MEIS2 | Body; Body; Body; | N_Shore | NA | II | 15 |
| | | ;MEIS2; MEIS2; MEIS | Body; Body Body; | | | | |
| | | 2; MEIS2 | Body | | | | |
| 1421 | cg13200838 | | | S_Shore | TRUE | II | 1 |
| 1422 | cg13207797 | EPO | TSS200 | Island | TRUE | I | 7 |
| 1423 | cg13208922 | | | Island | NA | I | 4 |
| 1424 | cg13211683 | GDF6 | Body | Island | NA | I | 8 |
| 1425 | cg13213923 | MARCH10; MARCH1 | 5′UTR; TSS1500; 1s | Island | NA | I | 17 |
| | | 0; MARCH10 | tExon | | | | |
| 1426 | cg13217661 | | | Island | NA | I | X |
| 1427 | cg13223227 | MAOB | Body | Island | NA | II | X |
| 1428 | cg13266435 | ANKRD3OB | TSS200 | Island | NA | I | 18 |
| 1429 | cg13281171 | SYCP2L | TSS200 | N_Shore | NA | II | 6 |
| 1430 | cg13294846 | | | Island | NA | II | 5 |
| 1431 | cg13295238 | EBF1 | Body | Island | TRUE | II | 5 |
| 1432 | cg13300273 | GPR25 | 1stExon | Island | TRUE | I | 1 |
| 1433 | cg13300630 | ROBO1; ROBO1; ROB | Body; Body; Body; | | TRUE | II | 3 |
| | | O1; ROBO1 | Body | | | | |
| 1434 | cg13340126 | SLC9A3 | Body | Island | NA | I | 5 |
| 1435 | cg13355574 | | | Island | NA | I | 10 |
| 1436 | cg13388277 | ZNF727 | TSS1500 | N_Shore | NA | II | 7 |
| 1437 | cg13406145 | | | N_Shore | TRUE | II | 15 |
| 1438 | cg13409070 | PTPRN2; PTPRN2; PT | Body; Body; Body | S_Shelf | NA | I | 7 |
| | | PRN2 | | | | | |
| 1439 | cg13421439 | ANKRD29 | Body | Island | NA | I | 18 |
| 1440 | cg13434638 | ITGA11 | Body | Island | NA | I | 15 |
| 1441 | cg13443950 | | | | TRUE | II | 4 |
| 1442 | cg13449257 | | | N_Shore | NA | I | 9 |
| 1443 | cg13450196 | | | | TRUE | II | 2 |
| 1444 | cg13469595 | | | Island | TRUE | II | 1 |
| 1445 | cg13486406 | | | Island | NA | II | 7 |
| 1446 | cg13492245 | FPGS; FPGS | Body; 5′UTR | S_Shore | NA | I | 9 |
| 1447 | cg13492364 | FMN2 | Body | S_Shore | NA | II | 1 |
| 1448 | cg13501793 | | | N_Shore | NA | II | 14 |
| 1449 | cg13507224 | OR10J3 | 1stExon | | NA | II | 1 |
| 1450 | cg13525088 | | | S_Shore | TRUE | I | 9 |
| 1451 | cg13539545 | | | Island | TRUE | I | 14 |
| 1452 | cg13552831 | TRPM5 | Body | Island | NA | II | 11 |
| 1453 | cg13564460 | | | S_Shelf | NA | II | 6 |
| 1454 | cg13564742 | FOXE1 | TSS1500 | N_Shore | NA | II | 9 |
| 1455 | cg13567349 | B3GAT1; B3GAT1 | Body; Body | S_Shore | NA | II | 11 |
| 1456 | cg13568106 | | | | TRUE | I | 17 |
| 1457 | cg13582793 | NLGNI | 5′UTR | | NA | II | 3 |
| 1458 | cg13594869 | FLRT2 | Body | | NA | II | 14 |
| 1459 | cg13611347 | NOL4 | Body | Island | TRUE | I | 18 |
| 1460 | cg13631572 | ADCY4 | TSS200 | Island | NA | I | 14 |
| 1461 | cg13631916 | FAM162B | TSS200 | Island | NA | II | 6 |
| 1462 | cg13634242 | | | Island | TRUE | I | 5 |
| 1463 | cg13644239 | TRIP13; BRD9; BRD9; | TSS1500; 5′UTR; B | N_Shore | NA | II | 5 |
| | | TRIP13; BRD9 | ody; TSS1500; Body | | | | |
| 1464 | cg13655674 | TBX15 | 5′UTR | Island | NA | II | 1 |
| 1465 | cg13675050 | | | N_Shore | TRUE | I | X |
| 1466 | cg13678973 | ELAVL4 | Body | Island | TRUE | I | 1 |
| 1467 | cg13679303 | | | S_Shore | NA | I | 9 |
| 1468 | cg13718418 | | | N_Shore | NA | I | 12 |
| 1469 | cg13721404 | NOL4 | Body | Island | TRUE | I | 18 |
| 1470 | cg13726724 | | | Island | TRUE | II | X |
| 1471 | cg13748267 | | | S_Shore | NA | II | X |
| 1472 | cg13756380 | | | Island | NA | I | 9 |
| 1473 | cg13758712 | ADAMTS1 | TSS1500 | S_Shore | NA | I | 21 |
| 1474 | cg13799986 | TMEM141 | Body | S_Shore | NA | II | 9 |
| 1475 | cg13800209 | MEIS2; MEIS2; MEIS2 | 1stExon; Body; Bod | Island | NA | I | 15 |
| | | ; MEIS2; MEIS2; MEIS | y; 5′UTR; Body; Bod | | | | |
| | | 2; MEIS2 | y; Body | | | | |
| 1476 | cg13810556 | RPL37A | TSS1500 | N_Shore | NA | I | 2 |
| 1477 | cg13818316 | RIPK4 | Body | N_Shore | NA | II | 21 |
| 1478 | cg13821176 | TRIB1 | Body | S_Shelf | NA | II | 8 |
| 1479 | cg13849454 | | | Island | NA | I | 9 |
| 1480 | cg13871400 | HMSD | TSS200 | | TRUE | I | 18 |
| 1481 | cg13875120 | AOX1; AOX1 | 1stExon; 5′UTR | Island | TRUE | I | 2 |
| 1482 | cg13875536 | BARHL1 | Body | Island | TRUE | I | 9 |
| 1483 | cg13877670 | ONECUT1 | TSS1500 | Island | TRUE | II | 15 |
| 1484 | cg13878677 | IQSEC1 | Body | | TRUE | I | 3 |
| 1485 | cg13886334 | | | N_Shore | NA | II | 2 |
| 1486 | cg13897627 | | | | NA | II | 16 |
| 1487 | cg13899188 | TRIM2; TRIM2 | Body; 5′UTR | Island | NA | I | 4 |
| 1488 | cg13912117 | | | Island | NA | I | 8 |
| 1489 | cg13925692 | CHL1 | 5′UTR | Island | NA | II | 3 |
| 1490 | cg13933262 | PCDHGA4; PCDHGA | Body; Body; Body; | Island | NA | I | 5 |
| | | 9; PCDHGA1; PCDHG | Body; TSS200; Bod | | | | |
| | | B1; CDHGB7; PCDH | y; Body; TSS200; Bo | | | | |
| | | GB6; PCDHGB3; PCD | dy; Body; Body; Bod | | | | |
| | | HGB7; PCDHGA6; PC | y; Body; Body; Body | | | | |
| | | DHGA8; PCDHGA10; | ; Body; Body; Body | | | | |
| | | PCDHGA5; PCDHGB4 | | | | | |
| | | ; PCDHGA3; PCDHGA | | | | | |
| | | 2; PCDHGB2; PCDHG | | | | | |
| | | A7; PCDHGB5 | | | | | |
| 1491 | cg13941830 | | | Island | NA | II | 1 |
| 1492 | cg13944838 | GFPT2 | Body | Island | NA | I | 5 |
| 1493 | cg13974838 | PTCH1; PTCH1; PTCH | Body; Body; Body; | | NA | II | 9 |
| | | 1; PTCH1; PTCH1; PTC | Body; Body Body; | | | | |
| | | H1; PTCH1 | Body | | | | |
| 1494 | cg13990092 | OXGR1 | TSS200 | Island | TRUE | I | 13 |
| 1495 | cg13994408 | MAGEA10; MAGEA1 | TSS1500; TSS1500 | | NA | II | X |
| | | 0 | | | | | |
| 1496 | cg14002960 | NOL4 | Body | Island | TRUE | I | 18 |
| 1497 | cg14015441 | DPYS | TSS200 | Island | NA | I | 8 |
| 1498 | cg14024242 | | | Island | NA | I | 9 |
| 1499 | cg14026459 | ELAVL2; ELAVL2; EL | 5′UTR; 5′UTR; 5′UT | Island | NA | I | 9 |
| | | AVL2 | R | | | | |
| 1500 | cg14030882 | | | | NA | I | 14 |
| 1501 | cg14033514 | | | S_Shore | NA | I | 17 |
| 1502 | cg14052511 | LOC389705 | Body | | NA | II | 9 |
| 1503 | cg14056738 | | | | TRUE | I | 2 |
| 1504 | cg14064694 | HIST1H2BI | 1stExon | Island | NA | II | 6 |
| 1505 | cg14065115 | | | N_Shore | NA | I | 6 |
| 1506 | cg14074052 | | | Island | NA | I | 12 |
| 1507 | cg14089267 | | | Island | NA | I | 13 |
| 1508 | cg14094639 | AURKAIP1; AURKAI | Body; Body; Body | Island | NA | I | 1 |
| | | P1; AURKAIP1 | | | | | |
| 1509 | cg14094983 | NXPH1 | Body | Island | NA | I | 7 |
| 1510 | cg14126020 | C20orf166; C20orf200 | 5′UTR; TSS1500 | N_Shore | NA | I | 20 |
| 1511 | cg14142965 | TXNRD1 | TSS200 | Island | NA | I | 12 |
| 1512 | cg14145801 | ZSWIM2 | 1stExon | N_Shore | TRUE | I | 2 |
| 1513 | cg14151317 | ABCA2; ABCA2 | Body; Body | N_Shore | NA | II | 9 |
| 1514 | cg14164044 | | | N_Shore | TRUE | II | 15 |
| 1515 | cg14179389 | GFI1; GFI1; GFI1 | Body; Body; Body | Island | NA | I | 1 |
| 1516 | cg14189141 | | | Island | NA | I | 9 |
| 1517 | cg14192029 | ZNF718 | Body | S_Shore | NA | II | 4 |
| 1518 | cg14202679 | | | S_Shore | NA | II | 6 |
| 1519 | cg14203525 | GAP43; GAP43 | Body; 5′UTR | Island | NA | I | 3 |
| 1520 | cg14206172 | FBXO17 | TSS1500 | S_Shore | TRUE | I | 19 |
| 1521 | cg14207921 | KLHL33 | Body | | NA | I | 14 |
| 1522 | cg14238325 | CRIP3 | TSS1500 | S_Shore | NA | II | 6 |
| 1523 | cg14247635 | ZAP70; ZAP70 | 3′UTR; 3′UTR | | NA | II | 2 |
| 1524 | cg14248367 | | | | TRUE | I | 9 |
| 1525 | cg14250833 | GCM2 | TSS200 | N_Shore | NA | I | 6 |
| 1526 | cg14254720 | LRRC8C | TSS1500 | N_Shore | NA | II | 1 |
| 1527 | cg14261840 | FEZF2 | TSS1500 | S_Shore | NA | II | 3 |
| 1528 | cg14262681 | | | N_Shore | NA | I | 20 |
| 1529 | cg14273822 | HIST1H3G; HIST1H2 | TSS200; TSS1500 | N_Shore | NA | II | 6 |
| | | B1 | | | | | |
| 1530 | cg14283569 | | | Island | NA | I | 19 |
| 1531 | cg14283758 | | | S_Shore | NA | II | 4 |
| 1532 | cg14299940 | PHYHD1; PHYHD1; P | Body; Body; Body | | NA | I | 9 |
| | | HYHD1 | | | | | |
| 1533 | cg14300812 | MED27 | Body | | TRUE | II | 9 |
| 1534 | cg14323199 | MRC2 | Body | Island | NA | II | 17 |
| 1535 | cg14326671 | LOC100192378 | Body | | TRUE | I | 8 |
| 1536 | cg14343214 | EYA4; EYA4; EYA4 | TSS1500; TSS1500 | N_Shore | NA | I | 6 |
| | | | ; TSS1500 | | | | |
| 1537 | cg14348741 | | | Island | NA | I | 9 |
| 1538 | cg14349667 | FGF5; FGF5 | Body; Body | S_Shore | NA | I | 4 |
| 1539 | cg14362370 | ABL1; ABL1 | 5′UTR; 1stExon | S_Shore | NA | II | 9 |
| 1540 | cg14376548 | CNP | Body | Island | NA | II | 17 |
| 1541 | cg14380270 | SLFN11; SLFN11; SLF | TSS200; TSS200; T | Island | TRUE | I | 17 |
| | | N11; SLFN11; SLFN11 | SS200; TSS200; TS | | | | |
| | | | S200 | | | | |
| 1542 | cg14395298 | | | Island | NA | I | 13 |
| 1543 | cg14399930 | FBXO47 | TSS200 | | NA | I | 17 |
| 1544 | cg14400354 | GALNTI2 | Body | S_Shore | NA | I | 9 |
| 1545 | cg14409023 | OTX2OS1 | Body | N_Shore | NA | I | 14 |
| 1546 | cg14428146 | NKX2-6 | TSS200 | Island | TRUE | I | 8 |
| 1547 | cg14448830 | | | N_Shore | NA | I | 9 |
| 1548 | cg14454796 | MTERFD2; MTERFD2 | Body; Body; 3′UTR; | | NA | I | 2 |
| | | ; SNED1; MTERFD2 | Body | | | | |
| 1549 | cg14454907 | | | N_Shelf | NA | II | 2 |
| 1550 | cg14478768 | HMHA1 | Body | Island | NA | II | 19 |
| 1551 | cg14480116 | SPRED2; SPRED2 | TSS1500; Body | | NA | II | 2 |
| 1552 | cg14482313 | KRT7 | TSS200 | Island | NA | I | 12 |
| 1553 | cg14503815 | | | | TRUE | II | 9 |
| 1554 | cg14511739 | APC; APC; APC | TSS200; TSS200; 5′ | | NA | I | 5 |
| | | | UTR | | | | |
| 1555 | cg14514403 | ARX | TSS200 | Island | NA | I | X |
| 1556 | cg14515517 | MYO3A | 5′UTR | S_Shelf | NA | II | 10 |
| 1557 | cg14537713 | | | N_Shore | NA | I | 6 |
| 1558 | cg14556896 | | | Island | NA | II | 12 |
| 1559 | cg14557185 | WWTR1; WWTR1; W | Body; Body; Body | Island | NA | I | 3 |
| | | WTR1 | | | | | |
| 1560 | cg14575053 | HMSD | TSS200 | | TRUE | I | 18 |
| 1561 | cg14576896 | GJD2 | 1stExon | Island | NA | I | 15 |
| 1562 | cg14578879 | SIM1 | Body | N_Shore | TRUE | II | 6 |
| 1563 | cg14603098 | ACCN1 | TSS1500 | Island | NA | I | 17 |
| 1564 | cg14604568 | PDE1C | 5′UTR | Island | NA | I | 7 |
| 1565 | cg14605874 | | | N_Shore | NA | II | 6 |
| 1567 | cg14613540 | HMSD | TSS1500 | | TRUE | II | 18 |
| 1568 | cg14616251 | GRHL3; GRHL3; GRH | TSS1500; Body; TS | Island | NA | II | 1 |
| | | L3 | S1500 | | | | |
| 1569 | cg14618996 | ADAM32 | TSS200 | N_Shore | NA | II | 8 |
| 1570 | cg14633455 | ZFP37 | TSS200 | Island | NA | I | 9 |
| 1571 | cg14642432 | A2BP1 A2BP1 | 5′UTR; 5′UTR | Island | NA | II | 16 |
| 1572 | cg14643001 | | | N_Shelf | TRUE | II | 3 |
| 1573 | cg14643390 | | | | TRUE | II | 7 |
| 1574 | cg14643537 | | | S_Shore | NA | II | 6 |
| 1575 | cg14646111 | SEC23B; SEC23B; SEC | 5′UTR; 5′UTR; 5′UT | S_Shore | NA | I | 20 |
| | | 23B | R | | | | |
| 1576 | cg14651249 | BARHL1 | 3′UTR | Island | TRUE | II | 9 |
| 1577 | cg14651254 | ATAD3A; ATAD3A; A | 5′UTR; Body; Body | S_Shore | NA | I | 1 |
| | | TAD3A | | | | | |
| 1578 | cg14663264 | | | Island | NA | I | 1 |
| 1579 | cg14666564 | HOXD3 | 5′UTR | S_Shore | NA | I | 2 |
| 1580 | cg14686949 | GABRE; MIR452 | Body; TSS1500 | | NA | II | X |
| 1581 | cg14696535 | HIST1H3G; HIST1H2 | TSS1500; TSS200 | N_Shore | NA | II | 6 |
| | | BI | | | | | |
| 1582 | cg14700679 | | | | TRUE | I | 2 |
| 1583 | cg14714797 | AHRR | Body | Island | NA | I | 5 |
| 1584 | cg14717557 | SIM1 | Body | N_Shore | TRUE | II | 6 |
| 1585 | cg14759284 | SRPX; SRPX; SRPX; S | TSS200; TSS200; T | S_Shore | NA | II | X |
| | | RPX | SS200; TSS200 | | | | |
| 1586 | cg14762784 | ATF6B; ATF6B; FKBP | TSS1500; TSS1500 | N_Shore | NA | II | 6 |
| | | L | ; Body | | | | |
| 1587 | cg14762973 | ZSWIM2 | TSS200 | Island | TRUE | II | 2 |
| 1588 | cg14765959 | | | Island | NA | I | 6 |
| 1589 | cg14776962 | HIST1H2BH; HIST1H | 1stExon; TSS1500 | Island | NA | II | 6 |
| | | 3F | | | | | |
| 1590 | cg14781189 | KCNH7; KCNH7; KCN | 1stExon; 5′UTR; 1st | | NA | I | 2 |
| | | H7; KCNH7 | Exon; 5′UTR | | | | |
| 1591 | cg14808890 | HIST1H2BH; HIST1H | TSS1500; 1stExon | Island | NA | II | 6 |
| | | 3F | | | | | |
| 1592 | cg14817655 | ZFP42 | TSS200 | Island | NA | I | 4 |
| 1593 | cg14824495 | SOX15 | 1stExon | Island | NA | II | 17 |
| 1594 | cg14825976 | C18orf1; C18orf1; C18o | TSS1500; Body; Bo | | TRUE | II | 18 |
| | | rf1; C18orf1 | dy; TSS1500 | | | | |
| 1595 | cg14826331 | CNP | Body | S_Shore | NA | I | 17 |
| 1596 | cg14843688 | | | | TRUE | II | 1 |
| 1597 | cg14846965 | C18orf1; C18orf1; C18o | TSS200; Body; Bod | | TRUE | I | 18 |
| | | rf1; C18orf1 | y; TSS200 | | | | |
| 1598 | cg14869505 | ATP5B; SNORD59B | Body; TSS200 | N_Shore | NA | II | 12 |
| 1599 | cg14871932 | GCK; GCK; GCK | Body; Body; Body | Island | NA | I | 7 |
| 1600 | cg14883291 | ME3; ME3; ME3 | TSS1500; TSS200; | S_Shore | NA | II | 11 |
| | | | TSS1500 | | | | |
| 1601 | cg14885748 | | | Island | TRUE | I | 12 |
| 1602 | cg14889643 | BCL2; BCL2 | Body; Body | Island | NA | I | 18 |
| 1603 | cg14903097 | C2orf43 | TSS1500 | S_Shore | NA | II | 2 |
| 1604 | cg14914046 | | | | TRUE | II | 6 |
| 1605 | cg14926717 | FLJ43390 | TSS200 | Island | NA | I | 14 |
| 1606 | cg14934253 | | | N_Shore | NA | II | 6 |
| 1607 | cg14943113 | | | S_Shore | TRUE | II | 14 |
| 1608 | cg14951132 | NRK | Body | Island | NA | I | X |
| 1609 | cg14959260 | SLC2A9; SLC2A9 | Body; Body | | NA | II | 4 |
| 1610 | cg14961598 | MTMR8 | Body | N_Shore | NA | I | X |
| 1611 | cg14962032 | CASP8; CASP8; CASP | 5′UTR; 5′UTR; 5′UT | | TRUE | II | 2 |
| | | 8; CASP8; CASP8 | R; 5′UTR; TSS200 | | | | |
| 1612 | cg14968543 | | | Island | NA | II | 8 |
| 1613 | cg14989988 | ATXN7L1; ATXN7L1 | Body; TSS1500 | | TRUE | II | 7 |
| 1614 | cg14996220 | ALX1 | TSS1500 | N_Shore | NA | I | 12 |
| 1615 | cg14997942 | | | | TRUE | II | 10 |
| 1616 | cg15012484 | | | Island | NA | I | 6 |
| 1617 | cg15013801 | ASCC1; C19orf104 | TSS1500; 5′UTR | S_Shore | NA | I | 10 |
| 1618 | cg15026277 | TMEM106A | TSS200 | Island | NA | I | 17 |
| 1619 | cg15028507 | C7orf50; C7orf50; C7or | Body; Body; Body | S_Shore | NA | I | 7 |
| | | f50 | | | | | |
| 1620 | cg15035437 | | | | NA | I | X |
| 1621 | cg15043384 | WWTR1; WWTR1; W | Body; Body; Body | Island | NA | I | 3 |
| | | WTR1 | | | | | |
| 1622 | cg15054725 | | | Island | NA | I | 2 |
| 1623 | cg15056128 | KRT222 | TSS200 | | NA | II | 17 |
| 1624 | cg15059608 | C3orf14 | TSS1500 | S_Shore | NA | II | 3 |
| 1625 | cg15060599 | | | Island | NA | I | 3 |
| 1626 | cg15077070 | IL1RL2 | TSS200 | N_Shore | NA | II | 2 |
| 1627 | cg15080119 | HIST1H2BH; HIST1H | 3′UTR; 1stExon; TS | S_Shore | NA | II | 6 |
| | | 2BH; HIST1H3F | S1500 | | | | |
| 1628 | cg15089705 | | | N_Shore | NA | II | 18 |
| 1629 | cg15095922 | NDUFS6; MRPL36 | TSS1500; TSS1500 | Island | NA | I | 5 |
| 1630 | cg15114474 | | | Island | TRUE | I | 11 |
| 1631 | cg15131808 | | | Island | NA | I | 5 |
| 1632 | cg15132013 | SKI | Body | S_Shelf | NA | II | 1 |
| 1633 | cg15133018 | | | Island | NA | I | 18 |
| 1634 | cg15140465 | PAK1; PAK1 | 5′UTR; 5′UTR | Island | NA | II | 11 |
| 1635 | cg15141467 | EFNA4; EFNA4; EFNA | TSS1500; TSS1500 | Island | NA | I | 1 |
| | | 4 | ; TSS1500 | | | | |
| 1636 | cg15160263 | | | Island | NA | I | 5 |
| 1637 | cg15162392 | QPCT | TSS200 | Island | TRUE | I | 2 |
| 1638 | cg15169940 | TBCB; POLR2I | TSS1500; 3′UTR | Island | NA | I | 19 |
| 1639 | cg15177902 | FBXO17; FBXO17 | 5′UTR; 1stExon | Island | TRUE | II | 19 |
| 1640 | cg15203028 | MUC6 | Body | Island | NA | I | 11 |
| 1641 | cg15215612 | | | Island | NA | I | 19 |
| 1642 | cg15219228 | TULP1 | Body | Island | TRUE | I | 6 |
| 1643 | cg15243542 | PURB; PURB | 3′UTR; 1stExon | | NA | II | 7 |
| 1644 | cg15265102 | | | N_Shore | NA | II | 17 |
| 1645 | cg15266705 | | | Island | NA | I | 12 |
| 1646 | cg15267232 | GATA3; GATA3 | Body; Body | Island | NA | I | 10 |
| 1647 | cg15285250 | NXPH1 | Body | Island | NA | II | 7 |
| 1648 | cg15291243 | LHX1 | Body | Island | NA | II | 17 |
| 1649 | cg15292275 | | | Island | NA | II | 13 |
| 1650 | cg15339075 | PARK2; PARK2; PAR | Body; Body; Body | | TRUE | II | 6 |
| | | K2 | | | | | |
| 1651 | cg15345437 | SNORD22; SNORD31; | TSS1500; Body; Bo | N_Shelf | NA | II | 11 |
| | | SNHG1 | dy | | | | |
| 1652 | cg15368905 | HTR1B | 1stExon | Island | NA | I | 6 |
| 1653 | cg15375469 | CPVL; CPVL | TSS200; TSS200 | | TRUE | II | 7 |
| 1654 | cg15379185 | EVX1 | 1stExon | Island | TRUE | I | 7 |
| 1655 | cg15386434 | MXRA8 | Body | Island | TRUE | I | 1 |
| 1656 | cg15386964 | HIST1H2BH; HIST1H | TSS1500; 1stExon | Island | NA | I | 6 |
| | | 3F | | | | | |
| 1657 | cg15392549 | | | | TRUE | II | 6 |
| 1658 | cg15394142 | HMGA2; HMGA2 | Body; Body | | TRUE | II | 12 |
| 1659 | cg15398152 | TNXB | Body | | NA | II | 6 |
| 1660 | cg15400655 | ZNF718 | Body | S_Shore | NA | I | 4 |
| 1661 | cg15401418 | SEPT9; SEPT9; SEPT9; | 5′UTR; 5′UTR; Bod | | TRUE | I | 17 |
| | | SEPT9 | y; 1stExon | | | | |
| 1662 | cg15412759 | TFAP2D | Body | Island | NA | II | 6 |
| 1663 | cg15420720 | STC2 | Body | N_Shelf | TRUE | II | 5 |
| 1664 | cg15423357 | | | | TRUE | I | 2 |
| 1665 | cg15436123 | RGS12; RGS12; RGS12 | Body; Body; Body | | TRUE | I | 4 |
| 1666 | cg15442728 | C14orf159; C14orf159; | TSS200; TSS200; T | | NA | II | 14 |
| | | C14orf159; C14orf159; | SS200; TSS200; TS | | | | |
| | | C14orf159 | S1500 | | | | |
| 1667 | cg15445000 | MED1 | TSS1500 | S_Shore | NA | II | 17 |
| 1668 | cg15447787 | | | Island | NA | I | 15 |
| 1669 | cg15455643 | COL4A2 | Body | S_Shore | NA | II | 13 |
| 1670 | cg15465051 | | | | TRUE | II | 3 |
| 1671 | cg15468095 | PYCARD; PYCARD | TSS200; TSS200 | S_Shore | NA | II | 16 |
| 1672 | cg15473084 | | | Island | TRUE | II | 5 |
| 1673 | cg15496063 | CYS1 | TSS1500 | Island | NA | II | 2 |
| 1674 | cg15496864 | | | Island | TRUE | II | 10 |
| 1675 | cg15504461 | | | N_Shore | NA | II | 3 |
| 1676 | cg15511022 | | | N_Shelf | NA | II | X |
| 1677 | cg15523060 | MIBD3L1 | TSS1500 | | NA | II | 19 |
| 1678 | cg15534561 | | | | NA | II | 5 |
| 1679 | cg15580075 | NCRNA00167; PRDM | TSS1500; 5′UTR; 5′ | N_Shore | NA | II | 11 |
| | | 10; PRDMIO | UTR | | | | |
| 1680 | cg15595525 | GPR123 | 3′UTR | N_Shore | NA | II | 10 |
| 1681 | cg15610027 | DTNA; DTNA; DTNA; | TSS200; 5′UTR; 5′U | | TRUE | II | 18 |
| | | DTNA | TR; 5′UTR | | | | |
| 1682 | cg15611912 | RGS10 | Body | N_Shore | NA | II | 10 |
| 1683 | cg15621260 | FIBIN; FIBIN | 5′UTR; 1stExon | | TRUE | I | 11 |
| 1684 | cg15654259 | | | Island | TRUE | I | 1 |
| 1685 | cg15680288 | | | | TRUE | I | 4 |
| 1686 | cg15680620 | ANKRD45 | TSS200 | S_Shore | NA | II | 1 |
| 1687 | cg15682101 | | | S_Shelf | NA | II | 6 |
| 1688 | cg15727252 | SYT11 | Body | S_Shore | NA | II | 1 |
| 1689 | cg15732851 | ATP2B4; ATP2B4 | 5′UTR; 5′UTR | Island | NA | I | 1 |
| 1690 | cg15754153 | PLEKHH2 | 5′UTR | S_Shore | NA | II | 2 |
| 1691 | cg15759056 | ABCB1 | 5′UTR | Island | TRUE | I | 7 |
| 1692 | cg15778054 | KCTD14 | TSS200 | | NA | I | 11 |
| 1693 | cg15779837 | GRIN2D | Body | Island | TRUE | I | 19 |
| 1694 | cg15790037 | ARL5C | Body | Island | NA | I | 17 |
| 1695 | cg15814504 | | | Island | NA | II | 5 |
| 1696 | cg15827779 | CDC42BPB | TSS1500 | S_Shore | NA | I | 14 |
| 1697 | cg15843567 | MYO3A; MYO3A | 1stExon; 5′UTR | Island | NA | I | 10 |
| 1698 | cg15850390 | CDX4 | Body | S_Shore | TRUE | I | X |
| 1699 | cg15866977 | NOTUM | TSS1500 | Island | NA | I | 17 |
| 1700 | cg15874629 | MAOB; MAOB | 1stExon; 5′UTR | Island | NA | I | X |
| 1701 | cg15904664 | ASPG | Body | Island | NA | I | 14 |
| 1702 | cg15910208 | KLK10; KLK10; KLK1 | 5′UTR; 5′UTR; 5′UT | Island | NA | II | 19 |
| | | 0 | R | | | | |
| 1703 | cg15916192 | | | Island | TRUE | II | 12 |
| 1704 | cg15920791 | | | Island | NA | I | 8 |
| 1705 | cg15932013 | FLJ22536 | TSS200 | Island | NA | I | 6 |
| 1706 | cg15952370 | OR8D2 | TSS1500 | | NA | II | 11 |
| 1707 | cg15953310 | | | Island | TRUE | I | X |
| 1708 | cg15959715 | GAD2; GAD2 | Body; Body | Island | NA | I | 10 |
| 1709 | cg15963552 | C3orf26; FILIP1L; MIR | Body; Body; Body; 1 | N_Shore | NA | I | 3 |
| | | 548G; FILIP1L; FILIP1 | stExon; 5′UTR; Bod | | | | |
| | | L; FILIP1L | Y | | | | |
| 1710 | cg15965006 | STK19; STK19; STK19 | TSS200; TSS1500; | N_Shore | NA | I | 6 |
| | | ; DOM3Z | TSS1500; Body | | | | |
| 1711 | cg15972331 | FGF14; FGF14 | Body; TSS1500 | S_Shore | NA | I | 13 |
| 1712 | cg15973954 | | | | NA | II | 13 |
| 1713 | cg15982972 | | | | NA | II | 5 |
| 1714 | cg15984661 | CCDC8; CCDC8 | 1stExon; 5′UTR | N_Shore | NA | II | 19 |
| 1715 | cg15992535 | NRG2; NRG2; NRG2; N | Body; Body; Body; | Island | NA | I | 5 |
| | | RG2 | Body | | | | |
| 1716 | cg15994026 | CD38 | Body | Island | TRUE | I | 4 |
| 1717 | cg16045423 | APOBEC3B | TSS1500 | | TRUE | II | 22 |
| 1718 | cg16072304 | | | S_Shore | NA | II | 5 |
| 1719 | cg16101148 | | | S_Shore | NA | II | 12 |
| 1720 | cg16111259 | MAGI2 | Body | | TRUE | II | 7 |
| 1721 | cg16117799 | PRDM8 | 5′UTR | S_Shore | NA | I | 4 |
| 1722 | cg16132520 | AKR1B1 | TSS200 | Island | NA | I | 7 |
| 1723 | cg16133088 | EFCAB4B; EFCAB4B; | TSS200; TSS200; T | Island | TRUE | I | 12 |
| | | EFCAB4B | SS200 | | | | |
| 1724 | cg16167741 | NFYC; NFYC; NFYC; | 5′UTR; 5′UTR; 5′UT | | NA | II | 1 |
| | | NFYC; NFYC | R; 5′UTR; 5′UTR | | | | |
| 1725 | cg16182936 | IQSEC1 | Body | | TRUE | I | 3 |
| 1726 | cg16195091 | LOC283999 | Body | N_Shore | NA | I | 17 |
| 1727 | cg16201038 | CDX2 | TSS1500 | Island | TRUE | I | 13 |
| 1728 | cg16201883 | FSCN2; FSCN2 | 1stExon; 1stExon | Island | NA | I | 17 |
| 1729 | cg16215203 | ADCY4 | TSS200 | Island | NA | I | 14 |
| 1730 | cg16246242 | | | | TRUE | II | 7 |
| 1731 | cg16258337 | | | N_Shore | TRUE | II | 4 |
| 1732 | cg16259229 | | | | NA | II | 5 |
| 1733 | cg16289929 | | | N_Shore | NA | II | 5 |
| 1734 | cg16296754 | | | N_Shore | TRUE | II | 17 |
| 1735 | cg16300300 | | | N_Shore | NA | I | 13 |
| 1736 | cg16303918 | FEV | Body | N_Shore | NA | II | 2 |
| 1737 | cg16312163 | ALDHIL1; ALDHIL1 | 5′UTR; 1stExon | Island | TRUE | II | 3 |
| 1738 | cg16322479 | EXOC3; C5orf55 | 5′UTR; TSS1500 | S_Shore | NA | II | 5 |
| 1739 | cg16329197 | | | Island | TRUE | II | 12 |
| 1740 | cg16332224 | MOCS3; DPM1 | 1stExon; TSS1500 | S_Shore | NA | II | 20 |
| 1741 | cg16354520 | LCE2A | 3′UTR | | NA | II | 1 |
| 1742 | cg16357179 | DKFZp434L192 | TSS1500 | | NA | II | 7 |
| 1743 | cg16366686 | SEPT9; SEPT9; SEPT9; | 5′UTR; 5′UTR; Bod | | TRUE | II | 17 |
| | | SEPT9 | y; 1stExon | | | | |
| 1744 | cg16373000 | C7orf50; C7orf50; C7or | Body; Body; Body | S_Shore | NA | II | 7 |
| | | f50 | | | | | |
| 1745 | cg16377609 | | | S_Shore | NA | II | 7 |
| 1746 | cg16382584 | 11-Sep | Body | | TRUE | II | 4 |
| 1747 | cg16401529 | PABPC5; PABPC5 | 5′UTR; 1stExon | Island | NA | I | X |
| 1748 | cg16406225 | | | N_Shore | TRUE | II | 14 |
| 1749 | cg16413687 | ALX1 | TSS1500 | N_Shore | NA | I | 12 |
| 1750 | cg16413715 | UNC5A | Body | Island | NA | II | 5 |
| 1751 | cg16414561 | ARX; ARX | 1stExon; 5′UTR | Island | NA | I | X |
| 1752 | cg16448058 | TLX1NB | 5′UTR | N_Shelf | NA | I | 10 |
| 1753 | cg16459265 | C7orf40; SNORA9 | Body; Body | N_Shore | NA | II | 7 |
| 1754 | cg16462006 | | | | TRUE | II | 14 |
| 1755 | cg16469740 | HDAC9; HDAC9 | Body; Body | | TRUE | II | 7 |
| 1756 | cg16473141 | REC8; REC8; REC8; RE | 1stExon; 1stExon; 5′ | Island | TRUE | II | 14 |
| | | C8 | UTR; 5′UTR | | | | |
| 1757 | cg16475478 | | | | NA | II | 7 |
| 1758 | cg16490191 | UNKL | 5′UTR | N_Shore | TRUE | II | 16 |
| 1759 | cg16507965 | ARX | TSS200 | Island | NA | I | X |
| 1760 | cg16509851 | | | Island | NA | I | 4 |
| 1761 | cg16515238 | FAM70A; FAM70A; F | Body; Body; Body | Island | NA | I | X |
| | | AM70A | | | | | |
| 1762 | cg16524733 | TAGLN; TAGLN; TAG | 5′UTR; 1stExon; 5′U | | NA | I | 11 |
| | | LN; TAGLN | TR; 1stExon | | | | |
| 1763 | cg16530382 | FBXO47 | TSS200 | | NA | II | 17 |
| 1764 | cg16539957 | | | N_Shore | NA | II | 14 |
| 1765 | cg16559570 | RTEL1; RTEL1 | Body; Body | Island | NA | I | 20 |
| 1766 | cg16565394 | SIM1 | Body | N_Shore | NA | II | 6 |
| 1767 | cg16570157 | EFCAB4A | Body | Island | NA | I | 11 |
| 1768 | cg16589135 | | | Island | NA | I | 3 |
| 1769 | cg16590821 | LONRF3; LONRF3 | TSS1500; TSS1500 | Island | NA | I | X |
| 1770 | cg16596691 | SNRPA | Body | S_Shelf | NA | II | 19 |
| 1771 | cg16618605 | GABRE; M1R452 | Body; TSS200 | | NA | II | X |
| 1772 | cg16620382 | TTBK1 | TSS200 | Island | NA | I | 6 |
| 1773 | cg16640855 | NEUROD1 | Body | | TRUE | I | 2 |
| 1774 | cg16643088 | CSRNP1 | 5′UTR | S_Shelf | TRUE | II | 3 |
| 1775 | cg16653620 | | | Island | NA | II | 2 |
| 1776 | cg16664405 | | | Island | NA | I | 10 |
| 1777 | cg16675381 | | | | NA | II | 6 |
| 1778 | cg16683770 | | | Island | TRUE | I | 20 |
| 1779 | cg16692973 | FAM70A; FAM70A; F | 1stExon; 5′UTR; 5′U | Island | NA | I | X |
| | | AM70A; FAM70A; FA | TR; 1stExon; 1stExo | | | | |
| | | M70A; FAM70A | n; 5′UTR | | | | |
| 1780 | cg16695483 | NID2 | TSS1500 | Island | NA | I | 14 |
| 1781 | cg16705697 | | | | NA | I | 11 |
| 1782 | cg16709294 | SFR55; SFR55; LOC10 | Body; Body; TSS15 | S_Shore | NA | I | 14 |
| | | 028951 | 00 | | | | |
| 1783 | cg16718276 | C1orf86; LOC1001280 | Body; TSS200; Bod | N_Shore | NA | I | 1 |
| | | 03; C1orf86 | y | | | | |
| 1784 | cg16718624 | ROB02; ROB02 | 1stExon; 5′UTR | S_Shore | NA | I | 3 |
| 1785 | cg16732616 | DMRTA2 | Body | Island | NA | I | 1 |
| 1786 | cg16733705 | | | | NA | I | 12 |
| 1787 | cg16739441 | KCNJ8 | Body | | NA | II | 12 |
| 1788 | cg16744973 | | | N_Shelf | NA | II | 20 |
| 1789 | cg16747109 | PITX2; PITX2; PITX2 | TSS1500; Body; Bo | Island | NA | II | 4 |
| | | | dy | | | | |
| 1790 | cg16749578 | NR2F6 | TSS1500 | S_Shore | NA | II | 19 |
| 1791 | cg16763089 | LOC149837 | TSS200 | | NA | II | 20 |
| 1792 | cg16768018 | ZIC4; ZIC4; ZIC4; ZIC4 | Body; Body; Body; | Island | NA | I | 3 |
| | | ; ZIC4 | Body; Body | | | | |
| 1793 | cg16772207 | MYT1 | TSS200 | | NA | II | 20 |
| 1794 | cg16773115 | TPD52; TPD52; TPD52 | Body; 5′UTR; 1stEx | | TRUE | II | 8 |
| | | ; TPD52 | on; Body | | | | |
| 1795 | cg16773899 | EDIL3; EDIL3 | 5′UTR; 1stExon | Island | NA | II | 5 |
| 1796 | cg16800165 | FLRT2 | TSS200 | N_Shore | NA | II | 14 |
| 1797 | cg16803596 | GATA4 | TSS1500 | Island | TRUE | I | 8 |
| 1798 | cg16804284 | FOXG1 | 1stExon | Island | NA | II | 14 |
| 1799 | cg16819028 | PKP1; PKP1 | TSS1500; TSS1500 | N_Shore | NA | II | 1 |
| 1800 | cg16831623 | BMP2 | 5′UTR | N_Shore | NA | II | 20 |
| 1801 | cg16833110 | OR2D3 | TSS200 | | NA | II | 11 |
| 1802 | cg16842053 | CARD11; CARD11 | 1stExon; 5′UTR | | TRUE | I | 7 |
| 1803 | cg16855845 | DNASE2 | Body | N_Shore | NA | II | 19 |
| 1804 | cg16869108 | VHL; VHL | Body; Body | S_Shore | NA | II | 3 |
| 1805 | cg16894819 | | | N_Shelf | NA | II | 1 |
| 1806 | cg16927242 | MEIS1 | Body | S_Shelf | NA | II | 2 |
| 1807 | cg16968020 | GABRA3 | Body | | NA | II | X |
| 1808 | cg16998564 | | | | TRUE | II | 1 |
| 1809 | cg17004353 | CARS; CARS; CARS; C | Body; Body; 5′UTR; | | NA | I | 11 |
| | | ARS | Body | | | | |
| 1810 | cg17032646 | SLC6A17 | TSS1500 | N_Shore | TRUE | II | 1 |
| 1811 | cg17034390 | | | Island | NA | I | 6 |
| 1812 | cg17052266 | CPVL; CPVL | TSS200; TSS200 | | TRUE | II | 7 |
| 1813 | cg17071446 | | | N_Shore | TRUE | II | 4 |
| 1814 | cg17080882 | TGFBR3 | Body | | TRUE | II | 1 |
| 1815 | cg17084751 | ZNF215 | Body | | NA | II | 11 |
| 1816 | cg17093995 | VWC2 | Body | Island | NA | I | 7 |
| 1817 | cg17094985 | | | Island | TRUE | I | 3 |
| 1818 | cg17098995 | CAT | 3′UTR | | NA | I | 11 |
| 1819 | cg17099568 | | | N_Shore | NA | II | 8 |
| 1820 | cg17100761 | EPO | TSS1500 | N_Shore | TRUE | I | 7 |
| 1821 | cg17101165 | | | Island | NA | II | 12 |
| 1822 | cg17105834 | ASTN1; ASTN1 | 1stExon; 1stExon | Island | TRUE | I | 1 |
| 1823 | cg17109725 | | | S_Shelf | NA | II | 1 |
| 1824 | cg17110524 | | | Island | NA | II | 6 |
| 1825 | cg17160382 | GJA1 | 5′UTR | | NA | I | 6 |
| 1826 | cg17160886 | C6orf141 | 1stExon | Island | TRUE | II | 6 |
| 1827 | cg17163760 | CPVL; CPVL | TSS200; TSS200 | | TRUE | II | 7 |
| 1828 | cg17177455 | | | Island | NA | II | 2 |
| 1829 | cg17201760 | ZNF876P | Body | Island | NA | II | 4 |
| 1830 | cg17202313 | SOX2OT | Body | Island | NA | I | 3 |
| 1831 | cg17214381 | ZFP42 | TSS1500 | Island | NA | I | 4 |
| 1832 | cg17214408 | | | | NA | I | X |
| 1833 | cg17235702 | | | Island | NA | II | 7 |
| 1834 | cg17237063 | RBMS3; RBMS3; RBM | 1stExon; 1stExon; 1 | | NA | II | 3 |
| | | S3 | stExon | | | | |
| 1835 | cg17241776 | DLEU7 | 1stExon | Island | NA | I | 13 |
| 1836 | cg17242351 | ACTC1 | Body | | NA | I | 15 |
| 1837 | cg17266581 | MBP; MBP | Body; Body | Island | NA | I | 18 |
| 1838 | cg17276590 | EMX2OS; EMX2; EM | Body; Body; Body | Island | NA | I | 10 |
| | | X2 | | | | | |
| 1839 | cg17280346 | ZIC1 | TSS1500 | N_Shore | NA | I | 3 |
| 1840 | cg17284804 | TCTEXID1 | TSS200 | Island | TRUE | I | 1 |
| 1841 | cg17292384 | COX8A | 3′UTR | S_Shore | NA | II | 11 |
| 1842 | cg17302155 | PRDM13 | Body | Island | NA | I | 6 |
| 1843 | cg17305181 | CRH; CRH | 1stExon; 5′UTR | S_Shore | NA | I | 8 |
| 1844 | cg17315500 | | | Island | TRUE | I | 12 |
| 1845 | cg17332573 | HIST1H2BH; HIST1H | TSS1500; TSS200 | N_Shore | NA | II | 6 |
| | | 3F | | | | | |
| 1846 | cg17342759 | PPP2R2B; PPP2R2B | TSS1500; TSS1500 | | NA | II | 5 |
| 1847 | cg17344516 | | | Island | TRUE | I | 5 |
| 1848 | cg17361163 | EMID2 | TSS200 | Island | NA | II | 7 |
| 1849 | cg17374727 | LUZP6; MTPN | 5′UTR; Body | | TRUE | II | 7 |
| 1850 | cg17380661 | SIM1 | TSS1500 | Island | NA | I | 6 |
| 1851 | cg17385175 | | | Island | NA | I | X |
| 1852 | cg17392573 | | | Island | NA | I | 6 |
| 1853 | cg17394649 | HCG4 | Body | Island | NA | I | 6 |
| 1854 | cg17407511 | SRPX; SRPX; SRPX; S | TSS200; TSS200; T | S_Shore | NA | II | X |
| | | RPX | SS200; TSS200 | | | | |
| 1855 | cg17410236 | FLRT2; FLRT2 | 1stExon; 5′UTR | Island | NA | I | 14 |
| 1856 | cg17435901 | SLC39A7; RXRB; SLC | Body; TSS1500; Bo | S_Shore | NA | I | 6 |
| | | 39A7 | dy | | | | |
| 1857 | cg17440085 | | | | NA | II | 11 |
| 1858 | cg17448336 | | | N_Shore | NA | II | 3 |
| 1859 | cg17499941 | LOC645323 | Body | N_Shore | NA | II | 5 |
| 1860 | cg17529435 | | | Island | NA | I | X |
| 1861 | cg17544952 | MEOX2 | Body | | NA | II | 7 |
| 1862 | cg17546247 | ZIC4; ZIC1 | TSS1500; TSS1500 | N_Shore | TRUE | II | 3 |
| 1863 | cg17555825 | | | Island | NA | I | 5 |
| 1864 | cg17559156 | SLC43A1 | Body | S_Shelf | NA | II | 11 |
| 1865 | cg17560340 | RNMTL1; GLOD4 | TSS1500; Body | N_Shore | NA | II | 17 |
| 1866 | cg17562247 | FAM70A; FAM70A; F | Body; Body; Body | N_Shore | NA | II | X |
| | | AM70A | | | | | |
| 1867 | cg17569842 | RGS12; RGS12; RGS12 | Body; TSS200; Bod | N_Shelf | TRUE | II | 4 |
| 1868 | cg17570276 | EIF2AK1; EIF2AK1 | TSS1500; TSS1500 | S_Shore | NA | I | 7 |
| 1869 | cg17594860 | AKR7A3 | TSS200 | Island | NA | I | 1 |
| 1870 | cg17600231 | TSC22D1; TSC22D1 | 3′UTR; 3′UTR | N_Shelf | TRUE | II | 13 |
| 1871 | cg17602451 | BCL2; BCL2 | Body; Body | Island | NA | II | 18 |
| 1872 | cg17608490 | FEV | Body | N_Shore | NA | II | 2 |
| 1873 | cg17609804 | TBRG4; TBRG4; TBR | Body; Body; Body | N_Shelf | NA | II | 7 |
| | | G4 | | | | | |
| 1874 | cg17618327 | MADIL1; MADIL1; M | Body; Body; Body | | TRUE | I | 7 |
| | | ADIL1 | | | | | |
| 1875 | cg17630392 | NPR3 | 1stExon | Island | NA | II | 5 |
| 1876 | cg17632299 | LECT1; LECT1 | Body; Body | N_Shore | TRUE | I | 13 |
| 1877 | cg17641904 | C9orf4 | 1stExon | Island | NA | I | 9 |
| 1878 | cg17647537 | FITR3B | Body | | NA | II | 11 |
| 1879 | cg17647884 | GNRH1; GNRH1 | TSS200; TSS200 | | NA | II | 8 |
| 1880 | cg17665652 | ANK2; ANK2; ANK2 | Body; Body; Body | | TRUE | II | 4 |
| 1881 | cg17675712 | | | | TRUE | II | 2 |
| 1882 | cg17683110 | | | Island | NA | II | 16 |
| 1883 | cg17684207 | LOC285796 | TSS1500 | | NA | I | 6 |
| 1884 | cg17684657 | SYCP2L | TSS200 | N_Shore | NA | II | 6 |
| 1885 | cg17696160 | PAX9 | Body | Island | NA | II | 14 |
| 1886 | cg17705066 | GIPC3 | Body | Island | NA | I | 19 |
| 1887 | cg17711567 | LYPD5 | TSS1500 | Island | NA | II | 19 |
| 1888 | cg17717124 | TCL1A; TCL1A | TSS1500; TSS1500 | Island | NA | II | 14 |
| 1889 | cg17718302 | HIST1H3J | TSS200 | S_Shore | NA | II | 6 |
| 1890 | cg17720013 | IGFL4 | TSS1500 | | NA | II | 19 |
| 1891 | cg17721249 | ATP2B4; ATP2B4 | 5′UTR; 5′UTR | Island | TRUE | I | 1 |
| 1892 | cg17722002 | | | Island | NA | II | 7 |
| 1893 | cg17739038 | HIC1 | TSS1500 | Island | NA | I | 17 |
| 1894 | cg17740140 | TOP1 | Body | S_Shore | NA | II | 20 |
| 1895 | cg17759535 | NR5A2; NR5A2 | Body; Body | N_Shore | NA | II | 1 |
| 1896 | cg17795240 | GATA4 | Body | S_Shore | NA | II | 8 |
| 1897 | cg17797229 | | | S_Shore | TRUE | II | 13 |
| 1898 | cg17800442 | CTF1; CTF1 | Body; Body | S_Shelf | NA | II | 16 |
| 1899 | cg17804356 | NR5A2; NR5A2 | Body; Body | Island | NA | II | 1 |
| 1900 | cg17816394 | GNG4; GNG4; GNG4; | TSS1500; 1stExon; | Island | NA | I | 1 |
| | | GNG4; GNG4 | 5′UTR; 1stExon; 5′U | | | | |
| | | | TR | | | | |
| 1901 | cg17820591 | ENO3; ENO3 | TSS1500; TSS1500 | Island | NA | II | 17 |
| 1902 | cg17847344 | EBF1 | Body | S_Shore | TRUE | II | 5 |
| 1903 | cg17862447 | KLHL29 | 5′UTR | | TRUE | I | 2 |
| 1904 | cg17891759 | | | Island | NA | I | 4 |
| 1905 | cg17897699 | | | | NA | II | 8 |
| 1906 | cg17922359 | | | Island | NA | I | 4 |
| 1907 | cg17933583 | | | S_Shore | NA | I | 6 |
| 1908 | cg17933722 | | | Island | TRUE | II | 1 |
| 1909 | cg17939059 | LOC728661; CDK11B; | Body; Body; Body; | S_Shore | NA | II | 1 |
| | | CDK11B; CDK11B; C | Body; Body; Body; 5 | | | | |
| | | DK11B; CDK11B; CD | ′UTR | | | | |
| | | K11B | | | | | |
| 1910 | cg17941572 | | | Island | NA | I | 1 |
| 1911 | cg17980508 | IFI44L | TSS1500 | | NA | II | 1 |
| 1912 | cg17990771 | | | | TRUE | II | 6 |
| 1913 | cg18000886 | ARX | TSS200 | Island | NA | I | X |
| 1914 | cg18018027 | | | N_Shore | NA | I | 6 |
| 1915 | cg18022005 | | | N_Shelf | NA | II | X |
| 1916 | cg18032812 | | | S_Shore | NA | I | 7 |
| 1917 | cg18034737 | ZFP42 | TSS200 | Island | NA | II | 4 |
| 1918 | cg18044383 | | | Island | NA | I | 18 |
| 1919 | cg18062721 | VGLL4; VGLL4 | Body; Body | | TRUE | I | 3 |
| 1920 | cg18063733 | | | Island | TRUE | I | 5 |
| 1921 | cg18065318 | C4orf49; C4orf49 | 5′UTR; 1stExon | Island | NA | I | 4 |
| 1922 | cg18070676 | FLRT2 | 5′UTR | Island | NA | I | 14 |
| 1923 | cg18091469 | | | Island | TRUE | I | 2 |
| 1924 | cg18093372 | | | Island | NA | I | 2 |
| 1925 | cg18096722 | | | Island | NA | II | 8 |
| 1926 | cg18102233 | ARX | TSS200 | Island | NA | I | X |
| 1927 | cg18108623 | SLFN11; SLFN11; SLF | TSS1500; TSS1500 | Island | TRUE | II | 17 |
| | | N11; SLFN11; SLFN11 | ; TSS1500; TSS150 | | | | |
| | | | 0; TSS1500 | | | | |
| 1928 | cg18117600 | | | Island | NA | I | 15 |
| 1929 | cg18118147 | SYT3; SYT3; SYT3 | Body; Body; Body | Island | NA | I | 19 |
| 1930 | cg18118685 | GALNT13 | 5′UTR | Island | NA | I | 2 |
| 1931 | cg18127012 | | | | TRUE | II | 4 |
| 1932 | cg18128969 | NCAM2 | TSS1500 | Island | NA | II | 21 |
| 1933 | cg18130044 | | | N_Shore | NA | I | 20 |
| 1934 | cg18132916 | | | Island | TRUE | I | 6 |
| 1935 | cg18133357 | ADARB2 | Body | | NA | I | 10 |
| 1936 | cg18142856 | OR10A3 | TSS200 | | NA | II | 11 |
| 1937 | cg18147485 | KCNV1; KCNV1 | 5′UTR; 1stExon | Island | TRUE | I | 8 |
| 1938 | cg18177414 | | | Island | NA | I | 7 |
| 1939 | cg18186343 | M1R770; MEG3; MEG3 | TSS1500; Body; Bo | | NA | II | 14 |
| | | ; MEG3 | dy; Body | | | | |
| 1940 | cg18257955 | SNORD115-15; | Body; Body; TSS15 | | NA | II | 15 |
| | | SNORD115-21; | 00 | | | | |
| | | SNORDI15-22 | | | | | |
| 1941 | cg18297736 | NKX6-1 | Body | N_Shore | NA | II | 4 |
| 1942 | cg18327669 | SSTR1 | TSS1500 | N_Shore | NA | II | 14 |
| 1943 | cg18333041 | CLSTN2 | Body | | TRUE | II | 3 |
| 1944 | cg18342462 | C11orf88; C11orf88 | 1stExon; 1stExon | Island | NA | I | 11 |
| 1945 | cg18345826 | SYCP2L | TSS200 | N_Shore | NA | II | 6 |
| 1946 | cg18347921 | HLA-J; NCRNA00171 | Body; Body | Island | NA | I | 6 |
| 1947 | cg18355562 | | | | TRUE | II | 7 |
| 1948 | cg18358894 | | | Island | TRUE | I | 7 |
| 1949 | cg18388552 | | | | NA | I | 6 |
| 1950 | cg18395636 | RAB38 | TSS200 | S_Shore | NA | I | 11 |
| 1951 | cg18397279 | PCDH11X | 5′UTR | Island | NA | I | X |
| 1952 | cg18397998 | C1orf86; LOC1001280 | Body; TSS1500; Bo | N_Shore | NA | I | 1 |
| | | 03; C1orf86 | dy | | | | |
| 1953 | cg18401367 | | | Island | NA | I | 5 |
| 1954 | cg18405691 | OR4C15 | 1stExon | | NA | II | 11 |
| 1955 | cg18420599 | DTNA; DTNA; DTNA; | TSS200; 5′UTR; 5′U | | TRUE | II | 18 |
| | | DTNA | TR; 5′UTR | | | | |
| 1956 | cg18423469 | DLGAP1; DLGAP1 | Body; Body | N_Shelf | NA | II | 18 |
| 1957 | cg18435674 | KRT222 | Body | | TRUE | II | 17 |
| 1958 | cg18437710 | KLK10; KLK10; KLK1 | 5′UTR; 5′UTR; 1stE | S_Shore | NA | I | 19 |
| | | 0; KLK10 | xon; 5′UTR | | | | |
| 1959 | cg18447419 | SLC9A3 | Body | N_Shore | NA | I | 5 |
| 1960 | cg18460175 | ETV2 | TSS200 | N_Shore | NA | II | 19 |
| 1961 | cg18461584 | | | | TRUE | II | 7 |
| 1962 | cg18465515 | HIST1H2BI | TSS200 | N_Shore | NA | II | 6 |
| 1963 | cg18470427 | SLFN12L | Body | | NA | I | 17 |
| 1964 | cg18472159 | | | Island | NA | II | 5 |
| 1965 | cg18479593 | | | Island | NA | II | 14 |
| 1966 | cg18492126 | NCAM2 | TSS1500 | Island | NA | II | 21 |
| 1967 | cg18503031 | MITF; MITF; MITF | Body; Body; TSS20 | | NA | II | 3 |
| 1968 | cg18512515 | | | N_Shore | NA | I | 15 |
| 1969 | cg18524788 | | | N_Shore | TRUE | I | 7 |
| 1970 | cg18527241 | ATXN7L1; ATXN7L1; | Body; 1stExon; 5′U | | NA | II | 7 |
| | | ATXN7LI | TR | | | | |
| 1971 | cg18530551 | | | Island | NA | I | 5 |
| 1972 | cg18536496 | GPRASP1; GPRASP1; | TSS1500; TSS1500 | Island | NA | I | X |
| | | GPRASP1 | ; TSS1500 | | | | |
| 1973 | cg18595258 | TMEM138; CYBASC3 | TSS1500; 5′UTR; 5′ | N_Shore | NA | II | 11 |
| | | ; CYBASC3; TMEM13 | UTR; TSS1500 | | | | |
| | | 8 | | | | | |
| 1974 | cg18608369 | SLFN11; SLFN11; SLF | TSS200; TSS200; T | Island | TRUE | I | 17 |
| | | N11; SLFN11; SLFN11 | SS200; TSS200; TS | | | | |
| | | | S200 | | | | |
| 1975 | cg18631798 | ACOX3; ACOX3 | Body; Body | | NA | I | 4 |
| 1976 | cg18636509 | | | | TRUE | II | 6 |
| 1977 | cg18639180 | C14orf159; C14orf159; | 1stExon; 1stExon; 5′ | | TRUE | II | 14 |
| | | C14orf159; C14orf159; | UTR; 1stExon; 5′UT | | | | |
| | | C14orf159; C14orf159; | R; 5′UTR; TSS1500; | | | | |
| | | C14orf159; C14orf159; | 1stExon; 5′UTR | | | | |
| | | C14orf159 | | | | | |
| 1978 | cg18653451 | CCDC8 | TSS200 | S_Shore | NA | I | 19 |
| 1979 | cg18664869 | PCDHB15 | TSS200 | N_Shore | NA | I | 5 |
| 1980 | cg18673954 | INA | TSS1500 | N_Shore | NA | I | 10 |
| 1981 | cg18682423 | SLC46A3; SLC46A3 | TSS200; TSS200 | Island | NA | I | 13 |
| 1982 | cg18688293 | NMBR | 1stExon | N_Shore | NA | II | 6 |
| 1983 | cg18703601 | ROB01 | TSS1500 | S_Shore | NA | I | 3 |
| 1984 | cg18709349 | PNMAL2 | TSS200 | Island | NA | I | 19 |
| 1985 | cg18724430 | | | N_Shore | TRUE | I | 15 |
| 1986 | cg18735015 | SH3PXD2A | Body | Island | TRUE | II | 10 |
| 1987 | cg18748888 | TNRC6C; TNRC6C | 3′UTR; 3′UTR | S_Shore | NA | I | 17 |
| 1988 | cg18755783 | SPG20; SPG20; SPG20; | 5′UTR; 5′UTR; 5′UT | Island | NA | II | 13 |
| | | SPG20 | R; 5′UTR | | | | |
| 1989 | cg18766755 | SPRR2A | 5′UTR | | NA | II | 1 |
| 1990 | cg18769353 | PTN | TSS200 | | NA | II | 7 |
| 1991 | cg18782604 | SIM1 | Body | Island | TRUE | I | 6 |
| 1992 | cg18786593 | | | S_Shore | NA | I | 2 |
| 1993 | cg18786873 | ALX3 | Body | Island | TRUE | I | 1 |
| 1994 | cg18789887 | GALNTL2 | TSS200 | | TRUE | II | 3 |
| 1995 | cg18790597 | FAM184B | TSS200 | Island | TRUE | I | 4 |
| 1996 | cg18791205 | | | N_Shore | TRUE | II | 7 |
| 1997 | cg18794839 | KCNK2; KCNK2; KCN | TSS1500; TSS1500 | Island | NA | I | 1 |
| | | K2 | ; Body | | | | |
| 1998 | cg18805027 | | | N_Shore | NA | I | 14 |
| 1999 | cg18816826 | | | | NA | II | 1 |
| 2000 | cg18844382 | EFS; EFS | TSS200; TSS200 | Island | NA | I | 14 |
| 2001 | cg18854419 | | | Island | NA | I | 11 |
| 2002 | cg18856581 | MDGA2 | TSS1500 | Island | NA | II | 14 |
| 2003 | cg18857062 | CRIP3 | Body | Island | TRUE | I | 6 |
| 2004 | cg18865445 | | | Island | TRUE | I | 13 |
| 2005 | cg18881873 | | | Island | TRUE | I | 20 |
| 2006 | cg18886245 | TFAP2B | Body | S_Shore | NA | II | 6 |
| 2007 | cg18891712 | FILIP1 | Body | | TRUE | II | 6 |
| 2008 | cg18894648 | LOC645323; LOC6453 | Body; TSS200 | N_Shore | TRUE | II | 5 |
| | | 23 | | | | | |
| 2009 | cg18907180 | | | Island | NA | II | 14 |
| 2010 | cg18914211 | HLA-G | Body | Island | NA | I | 6 |
| 2011 | cg18922629 | SSBP1; FLJ40852 | Body; TSS1500 | S_Shore | NA | II | 7 |
| 2012 | cg18950778 | WT1; WT1; WT1; WT1 | Body; Body; Body; | Island | NA | II | 11 |
| | | | Body | | | | |
| 2013 | cg18954504 | | | N_Shore | TRUE | I | 17 |
| 2014 | cg18956714 | OXGR1 | TSS200 | S_Shore | TRUE | II | 13 |
| 2015 | cg18989085 | OPCML | Body | | TRUE | II | 11 |
| 2016 | cg19022384 | | | Island | NA | I | X |
| 2017 | cg19030994 | PDCD6 | Body | S_Shore | NA | I | 5 |
| 2018 | cg19045293 | | | Island | NA | I | X |
| 2019 | cg19045644 | IGFBP7 | TSS1500 | Island | NA | I | 4 |
| 2020 | cg19102771 | CCDC140 | 5′UTR | N_Shore | NA | II | 2 |
| 2021 | cg19106893 | PTPN23 | Body | S_Shelf | NA | II | 3 |
| 2022 | cg19113530 | LOC100130691; AGPS | TSS1500; Body | S_Shore | NA | I | 2 |
| 2023 | cg19155518 | GRIK2; GR1K2; GRIK2 | Body; Body; Body | Island | TRUE | I | 6 |
| 2024 | cg19162768 | | | N_Shore | NA | II | 15 |
| 2025 | cg19170660 | HCG18; HCG18 | Body; Body | N_Shore | NA | II | 6 |
| 2026 | cg19205850 | RAB7A | 5′UTR | S_Shore | NA | II | 3 |
| 2027 | cg19235071 | | | | TRUE | II | 6 |
| 2028 | cg19247350 | HIST1H2BH; HIST1H | TSS1500; TSS200 | N_Shore | NA | II | 6 |
| | | 3F | | | | | |
| 2029 | cg19307500 | HMHA1 | Body | Island | NA | II | 19 |
| 2030 | cg19330592 | Cl4orf159; C14orf159; | 1stExon; 1stExon; 5′ | | NA | I | 14 |
| | | C14orf159; C14orf159; | UTR; 1stExon; 5′UT | | | | |
| | | C14orf159; C14orf159; | R; 5′UTR; TSS1500; | | | | |
| | | C14orf159; C14orf159; | 1stExon; 5′UTR | | | | |
| | | C14orf159 | | | | | |
| 2031 | cg19345277 | ZIM3 | 3′UTR | | NA | II | 19 |
| 2032 | cg19350360 | | | Island | NA | II | 15 |
| 2033 | cg19376851 | FLJ32063 | Body | Island | TRUE | II | 2 |
| 2034 | cg19378036 | HNF1B; HNF1B | TSS1500; TSS1500 | Island | NA | I | 17 |
| 2035 | cg19380001 | DMRTA2 | TSS1500 | Island | NA | I | 1 |
| 2036 | cg19382808 | LOC728661; CDK11B; | Body; Body; Body; | Island | NA | II | 1 |
| | | CDK11B; CDK11B; C | Body; Body Body; 5 | | | | |
| | | DK11B; CDK11B; CD | ′UTR | | | | |
| | | K11B | | | | | |
| 2037 | cg19391498 | GALM | TSS1500 | | NA | II | 2 |
| 2038 | cg19426944 | COL2A1; COL2A1 | Body; Body | N_Shore | TRUE | I | 12 |
| 2039 | cg19481013 | | | Island | NA | II | X |
| 2040 | cg19500479 | GDF7 | TSS1500 | Island | TRUE | II | 2 |
| 2041 | cg19513207 | | | Island | NA | II | 3 |
| 2042 | cg19513940 | DOK6 | Body | Island | NA | I | 18 |
| 2043 | cg19526130 | LOC729467 | Body | Island | NA | II | 1 |
| 2044 | cg19535896 | ZSWIM2; ZSWIM2 | 1stExon; 5′UTR | Island | TRUE | I | 2 |
| 2045 | cg19542346 | CDK4 | Body | N_Shore | NA | II | 12 |
| 2046 | cg19550524 | | | | NA | II | 11 |
| 2047 | cg19556343 | NCAM2 | TSS1500 | Island | NA | II | 21 |
| 2048 | cg19578835 | | | Island | NA | I | 14 |
| 2049 | cg19592336 | ZNF389 | TSS200 | | TRUE | I | 6 |
| 2050 | cg19595012 | PRDM8 | TSS200 | S_Shore | NA | I | 4 |
| 2051 | cg19595402 | SRCIN1 | Body | Island | TRUE | I | 17 |
| 2052 | cg19604907 | | | | TRUE | II | 12 |
| 2053 | cg19614643 | | | | TRUE | II | 2 |
| 2054 | cg19623012 | | | Island | TRUE | I | 11 |
| 2055 | cg19624849 | HFE2; HFE2; HFE2; HF | 5′UTR; 5′UTR; 5′UT | Island | TRUE | I | 1 |
| | | E2 | R; Body | | | | |
| 2056 | cg19653212 | GUCA1A | Body | Island | NA | I | 6 |
| 2057 | cg19667811 | | | | NA | II | 10 |
| 2058 | cg19672997 | MDGA2 | TSS1500 | Island | NA | I | 14 |
| 2059 | cg19696441 | UBXN10 | TSS200 | Island | NA | I | 1 |
| 2060 | cg19697981 | NR2E1; NR2E1 | 5′UTR; 1stExon | Island | TRUE | II | 6 |
| 2061 | cg19715094 | TMEM25; TMEM25; T | Body; Body; Body; | S_Shore | NA | II | 11 |
| | | MEM25; TMEM25; TM | Body; Body; Body | | | | |
| | | EM25; TMEM25 | | | | | |
| 2062 | cg19726408 | SH3PXD2A | Body | Island | TRUE | I | 10 |
| 2063 | cg19731655 | MAP7D2; MAP7D2; M | TSS200; Body; Bod | Island | NA | I | X |
| | | AP7D2; MAP7D2 | y; Body | | | | |
| 2064 | cg19743254 | OPCML; OPCML | Body; Body | | TRUE | II | 11 |
| 2065 | cg19777067 | | | Island | NA | I | 4 |
| 2066 | cg19793376 | BARIIL1 | Body | Island | NA | II | 9 |
| 2067 | cg19807257 | | | Island | NA | I | 1 |
| 2068 | cg19810598 | | | Island | NA | I | 1 |
| 2069 | cg19811087 | | | Island | TRUE | I | 1 |
| 2070 | cg19811994 | RASSF1; RASSF1; RA | Body; Body; Body; 5 | N_Shore | NA | II | 3 |
| | | SSF1; RASSF1 | ′UTR | | | | |
| 2071 | cg19844704 | EMX2; EMX20S; EM | Body; TSS200; Bod | Island | NA | II | 10 |
| | | X2 | Y | | | | |
| 2072 | cg19851715 | HIST1H2BI | TSS200 | N_Shore | NA | II | 6 |
| 2073 | cg19859323 | | | Island | NA | I | X |
| 2074 | cg19868631 | VSTM2A | TSS1500 | Island | NA | I | 7 |
| 2075 | cg19870698 | | | Island | NA | I | 14 |
| 2076 | cg19879075 | SPOCK2; SPOCK2 | Body; Body | Island | NA | I | 10 |
| 2077 | cg19887700 | | | Island | NA | II | 12 |
| 2078 | cg19889626 | | | Island | NA | I | 8 |
| 2079 | cg19929126 | TRIL | TSS200 | Island | NA | I | 7 |
| 2080 | cg19930575 | GABRE | 3′UTR | | NA | II | X |
| 2081 | cg19945554 | | | Island | NA | I | 15 |
| 2082 | cg19961043 | CHAD; ACSF2 | TSS1500; Body | Island | TRUE | I | 17 |
| 2083 | cg19961480 | CNR1; CNR1; CNR1; C | TSS1500; TSS1500 | Island | NA | I | 6 |
| | | NR1 | ; TSS1500; TSS150 | | | | |
| | | | 0 | | | | |
| 2084 | cg19971716 | QRFPR | 1stExon | Island | TRUE | I | 4 |
| 2085 | cg19974227 | RGS12; RGS12; RGS12 | Body; 5′UTR; 1stEx | N_Shelf | TRUE | II | 4 |
| | | ; RGS12 | on; Body | | | | |
| 2086 | cg19988492 | DYRK1A; DYRK1A; D | Body; Body; Body; | | TRUE | II | 21 |
| | | YRK1A; DYRK1A; DY | Body; Body | | | | |
| | | RK1A | | | | | |
| 2087 | cg19989295 | REC8; REC8 | TSS200; TSS200 | Island | NA | I | 14 |
| 2088 | cg19991916 | PIGB | TSS1500 | N_Shore | NA | I | 15 |
| 2089 | cg19997502 | | | S_Shore | NA | I | 2 |
| 2090 | cg20000562 | SFTA3 | Body | S_Shore | TRUE | II | 14 |
| 2091 | cg20021336 | PARK2; PARK2; PAR | Body; Body; Body | | TRUE | II | 6 |
| | | K2 | | | | | |
| 2092 | cg20041088 | | | Island | NA | II | 1 |
| 2093 | cg20055426 | | | Island | TRUE | I | 12 |
| 2094 | cg20059337 | AFF3; AFF3 | Body; Body | | TRUE | II | 2 |
| 2095 | cg20072171 | FEZF2 | Body | Island | NA | I | 3 |
| 2096 | cg20081453 | | | N_Shore | TRUE | I | X |
| 2097 | cg20101352 | CPVL; CPVL | TSS200; TSS200 | | TRUE | II | 7 |
| 2098 | cg20103519 | DOK6 | Body | Island | NA | II | 18 |
| 2099 | cg20107395 | DOK5; DOK5 | 1stExon; 5′UTR | Island | NA | I | 20 |
| 2100 | cg20120208 | TLX1 | 3′UTR | S_Shore | NA | I | 10 |
| 2101 | cg20170777 | COX10 | TSS1500 | N_Shore | NA | II | 17 |
| 2102 | cg20171396 | | | N_Shore | TRUE | II | 4 |
| 2103 | cg20202246 | | | Island | TRUE | I | X |
| 2104 | cg20229496 | | | N_Shore | TRUE | I | 19 |
| 2105 | cg20248458 | C2orf43 | 5′UTR | Island | NA | I | 2 |
| 2106 | cg20258486 | LOC154822 | TSS200 | S_Shore | NA | II | 7 |
| 2107 | cg20275132 | ZSCAN12; ZSCAN12 | TSS1500; TSS1500 | S_Shore | NA | II | 6 |
| 2108 | cg20282814 | SLC2A1 | Body | N_Shore | NA | II | 1 |
| 2109 | cg20289913 | NPFFR2 | 1stExon | Island | NA | I | 4 |
| 2110 | cg20293609 | CYR61 | Body | S_Shore | NA | I | 1 |
| 2111 | cg20295992 | | | Island | NA | II | 6 |
| 2112 | cg20297199 | BMP4; BMP4 | 5′UTR; 5′UTR | Island | NA | II | 14 |
| 2113 | cg20302533 | POU6F2; POU6F2 | Body; Body | | NA | I | 7 |
| 2114 | cg20330472 | EYA4; EYA4; EYA4 | TSS1500; TSS1500 | N_Shore | NA | II | 6 |
| | | | ; TSS1500 | | | | |
| 2115 | cg20337384 | | | Island | NA | II | 7 |
| 2116 | cg20340866 | | | N_Shore | NA | I | 12 |
| 2117 | cg20365336 | | | | TRUE | II | 6 |
| 2118 | cg20370678 | | | Island | NA | I | 17 |
| 2119 | cg20381767 | OXGR1 | TSS200 | S_Shore | TRUE | I | 13 |
| 2120 | cg20382028 | MCM3AP | Body | N_Shore | NA | II | 21 |
| 2121 | cg20394609 | EMB | Body | N_Shore | NA | II | 5 |
| 2122 | cg20395967 | MIR663 | TSS1500 | Island | NA | I | 20 |
| 2123 | cg20399509 | C21orf57 | 3′UTR | Island | NA | I | 21 |
| 2124 | cg20399616 | BCAT1 | Body | Island | TRUE | I | 12 |
| 2125 | cg20415053 | CATSPER4 | Body | | TRUE | I | 1 |
| 2126 | cg20418725 | CASP8; CASP8; CASP | 5′UTR; 5′UTR; 5′UT | | NA | II | 2 |
| | | 8; CASP8; CASP8 | R; TSS1500; 5′UTR | | | | |
| 2127 | cg20426713 | SAMID13 | TSS1500 | N_Shore | NA | II | 1 |
| 2128 | cg20435267 | ZNF354B | Body | S_Shore | NA | II | 5 |
| 2129 | cg20435725 | | | N_Shore | NA | I | 2 |
| 2130 | cg20446252 | PASD1 | Body | | NA | II | X |
| 2131 | cg20449692 | CLDN11 | 1stExon | Island | TRUE | I | 3 |
| 2132 | cg20456954 | ARFGEF1 | Body | | TRUE | II | 8 |
| 2133 | cg20504856 | | | Island | NA | II | 6 |
| 2134 | cg20520804 | PFKP | Body | S_Shore | NA | I | 10 |
| 2135 | cg20555674 | ARL5C | Body | Island | NA | II | 17 |
| 2136 | cg20573931 | IL7 | Body | | NA | II | 8 |
| 2137 | cg20600210 | APCDDIL | TSS200 | Island | NA | I | 20 |
| 2138 | cg20615848 | C18orf34; C18orf34 | TSS1500; TSS1500 | S_Shore | NA | II | 18 |
| 2139 | cg20622056 | SLC7A3; SLC7A3 | TSS200; TSS200 | S_Shore | NA | II | X |
| 2140 | cg20623601 | EPAS1 | Body | Island | NA | II | 2 |
| 2141 | cg20640433 | LAMA2; LAMA2 | TSS200; TSS200 | | TRUE | II | 6 |
| 2142 | cg20647610 | WDR69 | TSS200 | Island | TRUE | I | 2 |
| 2143 | cg20658100 | KLF12 | 5′UTR | N_Shore | NA | I | 13 |
| 2144 | cg20668321 | GRIA1; GRIA1 | 3′UTR; 3′UTR | | NA | II | 5 |
| 2145 | cg20686203 | TM7SF2 | 3′UTR | N_Shore | NA | II | 11 |
| 2146 | cg20691205 | SPG20; SPG20; SPG20; | 5′UTR; 5′UTR; 5′UT | N_Shore | NA | II | 13 |
| | | SPG20 | R; 5′UTR | | | | |
| 2147 | cg20711765 | | | | NA | II | 8 |
| 2148 | cg20711828 | LAMA2; LAMA2 | TSS200; TSS200 | | TRUE | I | 6 |
| 2149 | cg20743365 | LHX9; LHX9 | Body; Body | Island | NA | II | 1 |
| 2150 | cg20755170 | POU4F3 | 3′UTR | Island | NA | I | 5 |
| 2151 | cg20774846 | DPYS | TSS200 | S_Shore | NA | II | 8 |
| 2152 | cg20801007 | EFCAB4A | Body | Island | NA | I | 11 |
| 2153 | cg20808234 | | | Island | NA | II | 6 |
| 2154 | cg20842326 | PTPRN2; PTPRN2; PT | Body; Body; Body | Island | NA | II | 7 |
| | | PRN2 | | | | | |
| 2155 | cg20852226 | MYO7A; MYO7A; MY | Body; Body; Body | Island | NA | I | 11 |
| | | O7A | | | | | |
| 2156 | cg20883831 | KCNH2; KCNH2; KCN | TSS200; Body; Bod | Island | TRUE | I | 7 |
| | | H2 | y | | | | |
| 2157 | cg20884605 | ALDH2 | Body | S_Shore | NA | II | 12 |
| 2158 | cg20885179 | VILL | Body | Island | NA | I | 3 |
| 2159 | cg20885782 | | | Island | NA | II | 4 |
| 2160 | cg20886017 | NEK11; NEK11 | Body; Body | | TRUE | II | 3 |
| 2161 | cg20891501 | SETA3 | Body | S_Shore | NA | II | 14 |
| 2162 | cg20908789 | | | S_Shore | NA | II | 8 |
| 2163 | cg20916523 | VHL; VHL | Body; Body | S_Shore | NA | I | 3 |
| 2164 | cg20933109 | | | Island | NA | II | 14 |
| 2165 | cg20936013 | | | Island | TRUE | I | 14 |
| 2166 | cg20939084 | ZIC4; ZIC4; ZIC4; ZIC4 | Body; Body; Body; | Island | NA | II | 3 |
| | | ; ZIC4 | Body; Body | | | | |
| 2167 | cg20950033 | ELHL33 | Body | | NA | I | 14 |
| 2168 | cg20981127 | NR2F6 | TSS1500 | S_Shore | NA | II | 19 |
| 2169 | cg21033440 | SIPA1; S1PA1 | Body; Body | Island | NA | I | 11 |
| 2170 | cg21051972 | | | Island | NA | II | 11 |
| 2171 | cg21052656 | AIP | TSS1500 | N_Shore | NA | II | 11 |
| 2172 | cg21064916 | | | N_Shore | NA | I | 11 |
| 2173 | cg21101743 | LOC646999 | Body | Island | TRUE | II | 7 |
| 2174 | cg21109265 | CRIP3 | TSS200 | Island | TRUE | II | 6 |
| 2175 | cg21117965 | SPEG | Body | | TRUE | I | 2 |
| 2176 | cg21120436 | NLGN1 | 5′UTR | | NA | II | 3 |
| 2177 | cg21144063 | | | | NA | II | 7 |
| 2178 | cg21146037 | TRIB1 | 3′UTR | | NA | II | 8 |
| 2179 | cg21146184 | | | Island | TRUE | I | 22 |
| 2180 | cg21158087 | | | Island | NA | I | 17 |
| 2181 | cg21179492 | MRPS2 | Body | S_Shore | NA | I | 9 |
| 2182 | cg21183256 | | | Island | TRUE | I | 8 |
| 2183 | cg21183523 | ZNF521 | Body | N_Shore | NA | II | 18 |
| 2184 | cg21196747 | | | | NA | II | 6 |
| 2185 | cg21205978 | APOL6 | TSS1500 | | NA | II | 22 |
| 2186 | cg21211480 | TMEM106A | TSS200 | Island | NA | I | 17 |
| 2187 | cg21253087 | SNAPC4 | Body | N_Shelf | NA | I | 9 |
| 2188 | cg21253459 | | | N_Shore | TRUE | II | 15 |
| 2189 | cg21263122 | SSTR1 | TSS200 | N_Shore | NA | II | 14 |
| 2190 | cg21268422 | BACE2; BACE2; BAC | Body; Body; Body | S_Shelf | NA | I | 21 |
| | | E2 | | | | | |
| 2191 | cg21283066 | | | | TRUE | II | 6 |
| 2192 | cg21291134 | APCS | 3′UTR | | NA | II | 1 |
| 2193 | cg21293934 | ANKRD30B | TSS200 | Island | NA | I | 18 |
| 2194 | cg21350115 | CALCRL; CALCRL | 1stExon; 5′UTR | | TRUE | II | 2 |
| 2195 | cg21359793 | KRT4 | TSS1500 | | NA | II | 12 |
| 2196 | cg21359950 | CRADD | Body | | TRUE | II | 12 |
| 2197 | cg21362390 | SLITRK2; SLITRK2; S | 5′UTR; 5′UTR; 5′UT | Island | NA | I | X |
| | | LITRK2; SLITRK2; SL | R; 5′UTR; 5′UTR; 5′ | | | | |
| | | ITRK2; SLITRK2; SLI | UTR; 5 | | | | |
| | | TRK2; SLITRK2; SLIT | ′UTR; 5′UTR; 5′UT | | | | |
| | | RK2 | R | | | | |
| 2198 | cg21383487 | | | Island | TRUE | I | 10 |
| 2199 | cg21392341 | TBX15 | 5′UTR | Island | TRUE | I | 1 |
| 2200 | cg21397588 | ATP2B4; ATP2B4 | 5′UTR; 5′UTR | S_Shore | NA | II | 1 |
| 2201 | cg21461649 | CFTR | TSS200 | | NA | II | 7 |
| 2202 | cg21466913 | MAGI1; MAGI1; MAG | Body; Body; Body | | TRUE | II | 3 |
| | | I1 | | | | | |
| 2203 | cg21476673 | HOXB8 | Body | Island | NA | I | 17 |
| 2204 | cg21485303 | | | Island | NA | II | 12 |
| 2205 | cg21507095 | TBX1; TBX1; TBX1 | Body; 3′UTR; Body | Island | TRUE | II | 22 |
| 2206 | cg21526205 | ST6GALNAC3; ST6G | Body; Body | Island | NA | I | 1 |
| | | ALNAC3 | | | | | |
| 2207 | cg21532325 | WDR86 | TSS1500 | Island | NA | I | 7 |
| 2208 | cg21558752 | KRT4 | TSS1500 | | NA | II | 12 |
| 2209 | cg21560142 | BBC3; BBC3; BBC3; B | Body; Body; Body; | N_Shore | TRUE | I | 19 |
| | | BC3 | Body | | | | |
| 2210 | cg21575509 | | | Island | NA | II | 3 |
| 2211 | cg21579726 | ABT1 | 3′UTR | S_Shore | NA | II | 6 |
| 2212 | cg21590238 | C12orf43 | TSS1500 | S_Shore | NA | II | 12 |
| 2213 | cg21595968 | | | Island | NA | I | X |
| 2214 | cg21620778 | WIPF1 | TSS1500 | S_Shore | NA | I | 2 |
| 2215 | cg21635854 | | | N_Shore | NA | I | 11 |
| 2216 | cg21642176 | RAD51AP2 | TSS200 | | NA | I | 2 |
| 2217 | cg21647584 | CT45A1 | 5′UTR | | NA | II | X |
| 2218 | cg21650962 | TFAP2B | Body | S_Shelf | TRUE | II | 6 |
| 2219 | cg21654383 | | | Island | NA | I | 2 |
| 2220 | cg21663122 | HIST1H2BH; HIST1H | TSS1500; TSS200 | N_Shore | NA | II | 6 |
| | | 3F | | | | | |
| 2221 | cg21705897 | HMGCLL1; HMGCLL | TSS200; TSS200 | Island | TRUE | I | 6 |
| | | 1 | | | | | |
| 2222 | cg21707187 | | | Island | TRUE | I | 12 |
| 2223 | cg21731375 | | | Island | NA | II | 14 |
| 2224 | cg21785034 | | | N_Shore | TRUE | II | 7 |
| 2225 | cg21797452 | LOC286467 | Body | Island | NA | II | X |
| 2226 | cg21817737 | NAALAD2 | TSS200 | | NA | II | 11 |
| 2227 | cg21819468 | PDPN; PDPN; PDPN; P | TSS1500; TSS1500 | Island | NA | I | 1 |
| | | DPN | ; 1stExon; 1stExon | | | | |
| 2228 | cg21821214 | | | Island | NA | II | 2 |
| 2229 | cg21822905 | PEX10; PEX10 | Body; Body | S_Shore | NA | I | 1 |
| 2230 | cg21839338 | FAM18A | Body | Island | NA | II | 16 |
| 2231 | cg21843616 | | | S_Shore | NA | I | 4 |
| 2232 | cg21868134 | HPDL; HPDL | 1stExon; 5′UTR | Island | NA | I | 1 |
| 2233 | cg21881253 | MIR34B; BTG4; MIR3 | TSS200; TSS1500; | Island | NA | I | 11 |
| | | 4C | TSS1500 | | | | |
| 2234 | cg21883598 | DUOX2 | Body | Island | NA | I | 15 |
| 2235 | cg21883802 | FBLN5; FBLN5 | 5′UTR; 1stExon | Island | NA | I | 14 |
| 2236 | cg21884231 | SLC7A14; SLC7A14 | 5′UTR; 1stExon | Island | NA | I | 3 |
| 2237 | cg21890239 | | | Island | NA | I | X |
| 2238 | cg21890726 | | | Island | TRUE | I | 15 |
| 2239 | cg21916358 | | | Island | NA | I | 21 |
| 2240 | cg21960265 | TNXB | Body | | TRUE | I | 6 |
| 2241 | cg21981270 | ALOX5 | Body | Island | NA | II | 10 |
| 2242 | cg21985631 | | | S_Shelf | TRUE | I | 4 |
| 2243 | cg22009464 | DTNA; DTNA; DTNA; | TSS200; 5′UTR; 5′U | | TRUE | II | 18 |
| | | DTNA | TR; 5′UTR | | | | |
| 2244 | cg22012543 | | | S_Shore | NA | II | X |
| 2245 | cg22020954 | | | | NA | II | 17 |
| 2246 | cg22035501 | APC; APC; APC | TSS200; TSS200; 5′ | | NA | I | 5 |
| | | | UTR | | | | |
| 2247 | cg22058855 | | | Island | TRUE | I | 6 |
| 2248 | cg22071194 | MOCS3; DPM1 | 1stExon; TSS1500 | Island | NA | II | 20 |
| 2249 | cg22091110 | | | Island | NA | II | 18 |
| 2250 | cg22135279 | | | | NA | II | 11 |
| 2251 | cg22152728 | | | Island | NA | I | 11 |
| 2252 | cg22160073 | TRIM34; TRIM34; TRI | 5′UTR; 5′UTR; TSS | | NA | II | 11 |
| | | M34; TRIM6- | 200; Body; 5′UTR | | | | |
| | | TRIM34; TRIM34 | | | | | |
| 2253 | cg22164577 | UBR3 | Body | S_Shore | NA | II | 2 |
| 2254 | cg22190437 | SPHKAP; SPHKAP | Body; Body | N_Shore | TRUE | I | 2 |
| 2255 | cg22198397 | SLC1A6 | Body | | NA | II | 19 |
| 2256 | cg22215784 | GPM6B | Body | N_Shelf | NA | II | X |
| 2257 | cg22237200 | ATP1B2 | Body | Island | TRUE | I | 17 |
| 2258 | cg22243996 | | | | NA | II | 6 |
| 2259 | cg22244418 | OGFOD1; NUDT21 | TSS1500; Body | N_Shore | NA | II | 16 |
| 2260 | cg22248052 | TFAP2B | Body | Island | NA | I | 6 |
| 2261 | cg22260952 | CHST11 | Body | S_Shore | NA | I | 12 |
| 2262 | cg22263031 | | | | NA | I | 15 |
| 2263 | cg22280258 | ALOX15 | TSS200 | S_Shore | NA | II | 17 |
| 2264 | cg22290648 | SYCN | 1stExon | Island | NA | I | 19 |
| 2265 | cg22294740 | LINGO3 | 5′UTR | Island | NA | I | 19 |
| 2266 | cg22303045 | | | N_Shore | NA | II | 6 |
| 2267 | cg22304522 | ADCY8 | Body | | TRUE | II | 8 |
| 2268 | cg22325530 | HVCN1; HVCN1 | TSS1500; 5′UTR | Island | TRUE | II | 12 |
| 2269 | cg22340072 | | | Island | NA | I | 8 |
| 2270 | cg22359606 | NEUROD1 | TSS200 | N _Shelf | TRUE | II | 2 |
| 2271 | cg22361859 | FBXO47; FBXO47 | 1stExon; 5′UTR | | NA | I | 17 |
| 2272 | cg22370379 | | | | TRUE | II | 6 |
| 2273 | cg22388634 | VSX1 | Body | Island | NA | I | 20 |
| 2274 | cg22389730 | FCRLB | Body | Island | NA | I | 1 |
| 2275 | cg22409854 | | | Island | NA | I | 6 |
| 2276 | cg22410478 | C6or1186 | Body | Island | TRUE | I | 6 |
| 2277 | cg22443762 | LHX9; LHX9 | Body; Body | Island | NA | II | 1 |
| 2278 | cg22445447 | RTF1 | Body | S_Shelf | NA | II | 15 |
| 2279 | cg22454011 | C18orf1; C18orf1; C18o | TSS1500; Body; Bo | | TRUE | II | 18 |
| | | rf1; C18orf1 | dy; TSS1500 | | | | |
| 2280 | cg22459630 | LBXCOR1 | Body | Island | NA | I | 15 |
| 2281 | cg22462983 | EYA4; EYA4; EYA4 | TSS1500; TSS1500 | N_Shore | NA | II | 6 |
| | | | ; TSS1500 | | | | |
| 2282 | cg22473637 | MCHR2; MCHR2 | TSS200; TSS200 | Island | TRUE | I | 6 |
| 2283 | cg22483095 | SBNO2; SBNO2 | Body; Body | Island | TRUE | I | 19 |
| 2284 | cg22489153 | | | Island | NA | II | 12 |
| 2285 | cg22491927 | CACNA1A; CACNA1 | 5′UTR; 1stExon; 1st | Island | NA | I | 19 |
| | | A; CACNA1A; CACN | Exon; 5′UTR | | | | |
| | | A1A | | | | | |
| 2286 | cg22496809 | TBX18 | Body | Island | NA | I | 6 |
| 2287 | cg22502502 | TRIM38 | TSS1500 | | TRUE | II | 6 |
| 2288 | cg22508969 | HMGCLL1; HMGCLL | TSS200; TSS200 | Island | TRUE | I | 6 |
| | | 1 | | | | | |
| 2289 | cg22512438 | TRH; TRH | 5′UTR; 1stExon | Island | NA | I | 3 |
| 2290 | cg22518208 | FLRT2 | 5′UTR | Island | NA | II | 14 |
| 2291 | cg22524657 | | | Island | TRUE | I | 1 |
| 2292 | cg22549870 | | | Island | NA | I | 15 |
| 2293 | cg22558291 | LOC646405 | Body | Island | NA | I | 13 |
| 2294 | cg22571664 | NPR3 | Body | Island | NA | II | 5 |
| 2295 | cg22605919 | C17orf104 | TSS1500 | Island | TRUE | I | 17 |
| 2296 | cg22612764 | FAM3B; FAM3B | TSS200; TSS200 | N_Shore | NA | I | 21 |
| 2297 | cg22618405 | | | S_Shore | TRUE | I | 2 |
| 2298 | cg22620221 | DPP6 | TSS200 | Island | NA | I | 7 |
| 2299 | cg22629987 | QPCT | TSS200 | Island | TRUE | II | 2 |
| 2300 | cg22630918 | C10orf2; C10orf2; C10o | 1stExon; 5′UTR; 1st | Island | NA | I | 10 |
| | | rf2; C1Oorf2 | Exon; 5′UTR | | | | |
| 2301 | cg22639011 | KCNA4 | 5′UTR | Island | NA | II | 11 |
| 2302 | cg22658957 | | | Island | NA | I | X |
| 2303 | cg22694818 | LHX8 | 5′UTR | Island | TRUE | I | 1 |
| 2304 | cg22711792 | DMRTA2 | 3′UTR | Island | NA | I | 1 |
| 2305 | cg22724998 | | | N_Shore | TRUE | I | 15 |
| 2306 | cg22732749 | PCDHB15 | TSS1500 | N_Shore | NA | II | 5 |
| 2307 | cg22751696 | C3orf14 | TSS1500 | S_Shore | NA | II | 3 |
| 2308 | cg22759800 | | | Island | NA | I | 15 |
| 2309 | cg22771759 | | | Island | NA | II | 13 |
| 2310 | cg22784386 | SIM1 | Body | N_Shore | TRUE | II | 6 |
| 2311 | cg22795345 | ARHGEF7; ARTIGEF7 | Body; Body; Body; | Island | NA | I | 13 |
| | | ; ARHGEF7; ARIIGEF | Body; Body | | | | |
| | | 7; ARHGEF7 | | | | | |
| 2312 | cg22796353 | | | Island | NA | I | 7 |
| 2313 | cg22797169 | IL1RL2 | TSS200 | N_Shore | TRUE | II | 2 |
| 2314 | cg22798707 | ELAVL2; ELAVL2; EL | TSS1500; TSS1500 | Island | NA | II | 9 |
| | | AVL2 | ; 5′UTR | | | | |
| 2315 | cg22809871 | FGF14 | Body | S_Shelf | NA | I | 13 |
| 2316 | cg22827864 | | | Island | NA | II | 6 |
| 2317 | cg22844669 | | | Island | NA | II | X |
| 2318 | cg22848598 | ADAM32 | TSS200 | Island | NA | I | 8 |
| 2319 | cg22865470 | | | Island | TRUE | I | 1 |
| 2320 | cg22870775 | | | S_Shore | NA | II | 6 |
| 2321 | cg22871668 | EYA4; EYA4; EYA4 | TSS200; TSS200; T | Island | NA | I | 6 |
| | | | SS200 | | | | |
| 2322 | cg22881652 | | | S_Shore | NA | I | 4 |
| 2323 | cg22894896 | MIR193A | TSS200 | Island | NA | I | 17 |
| 2324 | cg22961278 | PRDM8 | 5′UTR | S_Shore | NA | I | 4 |
| 2325 | cg22968966 | | | Island | NA | II | 16 |
| 2326 | cg22971920 | NAA10 | Body | N_Shore | NA | II | X |
| 2327 | cg22976218 | NEUROD1 | 5′UTR | N_Shelf | NA | I | 2 |
| 2328 | cg22979093 | PTN; PTN | 5′UTR; 1stExon | | NA | II | 7 |
| 2329 | cg23003500 | VIPR1 | TSS1500 | Island | TRUE | I | 3 |
| 2330 | cg23017915 | WWTR1; WWTR1; W | Body; Body; Body | N_Shore | NA | II | 3 |
| | | WTR1 | | | | | |
| 2331 | cg23026864 | PCDHA7; PCDHA12; P | Body; Body; Body; T | Island | NA | I | 5 |
| | | CDHA6; PCDHAC1; P | SS200; Body; Body; | | | | |
| | | CDHA10; PCDHA4; P | Body; Body; Body; | | | | |
| | | CDHA11; PCDHA8; P | Body; Body; Body; | | | | |
| | | CDHA6; PCDHA1; PC | Body; Body; Body; | | | | |
| | | DHA2; PCDHA1; PCD | Body; TSS200; Bod | | | | |
| | | HA9; PCDHA13; PCD | y | | | | |
| | | HA5; PCDHA3; PCDH | | | | | |
| | | AC1; PCDHA10 | | | | | |
| 2332 | cg23028941 | EYA4; EYA4; EYA4 | Body; Body; Body | | TRUE | II | 6 |
| 2333 | cg23037133 | OXGR1 | TSS1500 | S_Shore | TRUE | II | 13 |
| 2334 | cg23049429 | ATP11A; ATP11A | Body; Body | S_Shore | NA | I | 13 |
| 2335 | cg23049458 | L1TD1; L1TD1 | 5′UTR; 5′UTR | Island | NA | I | 1 |
| 2336 | cg23056713 | NTM | Body | | TRUE | I | 11 |
| 2337 | cg23067317 | GALNTL2 | TSS200 | | TRUE | II | 3 |
| 2338 | cg23075337 | NXPH1 | Body | N_Shore | NA | II | 7 |
| 2339 | cg23085662 | | | Island | NA | II | 18 |
| 2340 | cg23090824 | PDE7B; PDE7B | 1stExon; 5′UTR | | NA | II | 6 |
| 2341 | cg23095615 | FBXO30 | TSS1500 | Island | NA | II | 6 |
| 2342 | cg23097402 | DMRTA2 | 3′UTR | Island | NA | I | 1 |
| 2343 | cg23113963 | SCNN113 | TSS1500 | Island | NA | I | 16 |
| 2344 | cg23118086 | RASL12 | Body | Island | NA | I | 15 |
| 2345 | cg23125200 | GJB6; GJB6; GJB6; GJB | TSS200; TSS200; T | Island | TRUE | I | 13 |
| | | 6 | SS200; TSS1500 | | | | |
| 2346 | cg23141355 | | | Island | NA | I | 3 |
| 2347 | cg23158731 | COL14A1 | 5′UTR | Island | NA | I | 8 |
| 2348 | cg23161999 | | | N_Shore | TRUE | I | 2 |
| 2349 | cg23166590 | | | | NA | II | 8 |
| 2350 | cg23179456 | ADCY4 | TSS200 | Island | NA | I | 14 |
| 2351 | cg23180270 | RNF113A; NDUFA1 | TSS1500; Body | S_Shore | NA | II | X |
| 2352 | cg23189410 | ZIC4; ZIC1 | TSS1500; TSS1500 | N_Shore | TRUE | I | 3 |
| 2353 | cg23191956 | FOXG1 | TSS1500 | Island | TRUE | I | 14 |
| 2354 | cg23205387 | | | Island | NA | I | 7 |
| 2355 | cg23207876 | CASP14 | TSS200 | | NA | II | 19 |
| 2356 | cg23217097 | | | N_Shore | NA | I | 14 |
| 2357 | cg23218559 | AMH | Body | Island | NA | I | 19 |
| 2358 | cg23233404 | SYCP2L | Body | Island | NA | II | 6 |
| 2359 | cg23239574 | | | S_Shore | NA | I | 1 |
| 2360 | cg23244790 | PCDHGA4; PCDHGA | Body; Body; Body; | Island | NA | 1 | 5 |
| | | 11; PCDHGA11; PCDH | Body; Body; Body; | | | | |
| | | GA9; PCDHGA1; PCD | Body; 1stExon; Bod | | | | |
| | | HGB1; P CDHGB6; PC | y; Body; Body; Body | | | | |
| | | DHGA12; PCDHGB3; | ; Body; 1stExon; Bod | | | | |
| | | PCDHGB7; PCDHGA6 | y; Body; Body; Body | | | | |
| | | ; PCDHGA8; PCDHGA | ; Body; Body; Body | | | | |
| | | 10; PCDHGA12; PCDH | | | | | |
| | | GA5; PCDHGB4; PCD | | | | | |
| | | HGA3; PCDHGA2; PC | | | | | |
| | | DHGA7; PCDHGB2; P | | | | | |
| | | CDHGB5 | | | | | |
| 2361 | cg23296408 | Cl4orf159; C14orf159; | 1stExon; 1stExon; 5′ | | TRUE | II | 14 |
| | | C14orf159; C14orf159; | UTR; 1stExon; 5′UT | | | | |
| | | C14orf159; C14orf159; | R; 5′UTR; TSS1500; | | | | |
| | | C14orf159; C14orf159; | 1stExon; 5′UTR | | | | |
| | | C14orf159 | | | | | |
| 2362 | cg23299641 | KCNE1L | 1stExon | Island | TRUE | I | X |
| 2363 | cg23303246 | NTM | Body | | TRUE | I | 11 |
| 2364 | cg23317501 | UGT3A1 | Body | | TRUE | II | 5 |
| 2365 | cg23318812 | | | Island | TRUE | I | 12 |
| 2366 | cg23328194 | ADRA1A; ADRA1A; A | Body; Body; Body; | | NA | II | 8 |
| | | DRA1A; ADRA1A | Body | | | | |
| 2367 | cg23338503 | DOCK2 | Body | Island | NA | I | 5 |
| 2368 | cg23363971 | IKZF4; IKZF4 | 1stExon; 5′UTR | | TRUE | I | 12 |
| 2369 | cg23367107 | | | Island | NA | I | 12 |
| 2370 | cg23367608 | FAM162B | 1stExon | Island | NA | II | 6 |
| 2371 | cg23371754 | ANK2; ANK2; ANK2 | Body; Body; Body | | TRUE | II | 4 |
| 2372 | cg23398076 | MEIS1 | Body | S_Shelf | NA | I | 2 |
| 2373 | cg23424003 | EMID2 | TSS200 | Island | NA | I | 7 |
| 2374 | cg23424273 | hTR1B | TSS200 | Island | NA | I | 6 |
| 2375 | cg23438893 | | | N_Shore | TRUE | II | 14 |
| 2376 | cg23440636 | | | Island | NA | II | 5 |
| 2377 | cg23449696 | ZIC1 | TSS1500 | N_Shore | NA | I | 3 |
| 2378 | cg23468045 | | | | TRUE | II | 5 |
| 2379 | cg23473396 | | | Island | TRUE | II | X |
| 2380 | cg23477967 | TFPI; TFPI; TFPI; TFPI | 1stExon; 5′UTR; 1st | | TRUE | II | 2 |
| | | STK19; STK19; STK19 | Exon; 5′UTR | | | | |
| 2381 | cg23514417 | ; DOM3Z | TSS1500; TSS1500 | N_Shore | NA | II | 6 |
| | | | ; TSS1500; Body | | | | |
| 2382 | cg23517208 | | | S_Shore | NA | I | 5 |
| 2383 | cg23519637 | C7orf50; C7orf50; C7or | Body; Body; Body | S_Shore | TRUE | II | 7 |
| | | f50 | | | | | |
| 2384 | cg23522194 | PLA2G4C; PLA2G4C; | Body; Body; Body | Island | NA | I | 19 |
| | | PLA2G4C | | | | | |
| 2385 | cg23524195 | GFRA1; GFRA1 | TSS1500; TSS1500 | Island | NA | I | 10 |
| 2386 | cg23536473 | FERD3L | 1stExon | Island | TRUE | I | 7 |
| 2387 | cg23549902 | | | Island | NA | I | 7 |
| 2388 | cg23554497 | | | S_Shore | TRUE | II | 12 |
| 2389 | cg23609450 | WFDC10B; WFDC10B | Body; Body | | NA | II | 20 |
| 2390 | cg23610453 | | | Island | TRUE | I | 10 |
| 2391 | cg23614791 | ACSS3 | TSS200 | Island | NA | I | 12 |
| 2392 | cg23624008 | | | S_Shore | TRUE | I | 1 |
| 2393 | cg23626733 | PEX10; PEX10 | TSS1500; TSS1500 | N_Shore | NA | I | 1 |
| 2394 | cg23629166 | PEX10; PEX10 | TSS1500; TSS1500 | N_Shore | NA | I | 1 |
| 2395 | cg23637354 | | | | NA | II | 6 |
| 2396 | cg23642200 | NLGN1 | 5′UTR | | NA | II | 3 |
| 2397 | cg23656300 | | | | NA | II | 10 |
| 2398 | cg23658477 | | | Island | NA | I | 12 |
| 2399 | cg23660154 | | | Island | NA | I | 1 |
| 2400 | cg23699274 | LRRK2 | TSS1500 | N_Shore | NA | II | 12 |
| 2401 | cg23715830 | | | Island | NA | I | 3 |
| 2402 | cg23720064 | SIM1 | Body | N_Shore | TRUE | II | 6 |
| 2403 | cg23759189 | NTM | Body | | NA | II | 11 |
| 2404 | cg23771603 | MYO3A | 5′UTR | Island | NA | I | 10 |
| 2405 | cg23776012 | KRT222; KRT222 | 5′UTR; 1stExon | | NA | II | 17 |
| 2406 | cg23779047 | | | | NA | I | X |
| 2407 | cg23782616 | HMSD | TSS200 | | TRUE | I | 18 |
| 2408 | cg23792314 | BCAT1 | Body | Island | TRUE | II | 12 |
| 2409 | cg23807890 | WDR69; WDR69 | 1stExon; 5′UTR | Island | TRUE | I | 2 |
| 2410 | cg23809442 | FGF14; FGF14 | Body; TSS1500 | S_Shore | NA | I | 13 |
| 2411 | cg23829990 | UNC50 | Body | S_Shore | NA | II | 2 |
| 2412 | cg23855505 | CHST11 | Body | S_Shore | NA | I | 12 |
| 2413 | cg23875663 | PCSK2 | Body | S_Shelf | NA | II | 20 |
| 2414 | cg23881566 | SRRM4 | Body | | TRUE | II | 12 |
| 2415 | cg23882545 | CASP8; CASP8; CASP | 5′UTR; 5′UTR; 5′UT | | TRUE | II | 2 |
| | | 8; CASP8; CASP8 | R; 5′UTR; TSS200 | | | | |
| 2416 | cg23913995 | | | Island | NA | I | 20 |
| 2417 | cg23915769 | LVRN | TSS1500 | N_Shore | TRUE | II | 5 |
| 2418 | cg23928468 | SLC5A6; SLC5A6 | 5′UTR; Body | N_Shore | NA | II | 2 |
| 2419 | cg23952663 | LHX8 | Body | Island | NA | I | 1 |
| 2420 | cg23965214 | FBXO47 | TSS1500 | | NA | II | 17 |
| 2421 | cg24001644 | MAP7D2; MAP7D2; M | Body; Body; Body; 1 | Island | NA | I | X |
| | | AP7D2; MAP7D2; MA | stExon; 5′UTR | | | | |
| | | P7D2 | | | | | |
| 2422 | cg24002167 | EDIL3 | Body | N_Shore | NA | II | 5 |
| 2423 | cg24008884 | ALX4 | TSS1500 | S_Shore | NA | I | 11 |
| 2424 | cg24014143 | KLHL1; KLHL1; ATX | 1stExon; 5′UTR; Bo | Island | TRUE | I | 13 |
| | | N8OS | dy | | | | |
| 2425 | cg24027397 | SOX2OT | Body | S_Shore | TRUE | II | 3 |
| 2426 | cg24041078 | BTG4; MIR34C | TSS1500; TSS1500 | Island | NA | II | 11 |
| 2427 | cg24046411 | CYBA | TSS200 | S_Shore | NA | II | 16 |
| 2428 | cg24046888 | EPAS1 | Body | Island | NA | II | 2 |
| 2429 | cg24052239 | CACNA1F | Body | Island | NA | II | X |
| 2430 | cg24061208 | ANKRD30B; ANKRD | 1stExon; 5′UTR | S_Shore | NA | II | 18 |
| | | 30B | | | | | |
| 2431 | cg24067982 | TCERG1L | Body | N_Shore | NA | II | 10 |
| 2432 | cg24074153 | MDGA2; MDGA2 | Body; Body | | TRUE | II | 14 |
| 2433 | cg24104241 | ACTA1 | Body | Island | TRUE | II | 1 |
| 2434 | cg24106636 | NHLH2; NHLH2 | 5′UTR; 5′UTR | Island | TRUE | I | 1 |
| 2435 | cg24119607 | CXXC5 | 5′UTR | N_Shore | TRUE | II | 5 |
| 2436 | cg24146349 | INTS1 | Body | N_Shore | NA | II | 7 |
| 2437 | cg24149658 | CAPN6 | TSS200 | | NA | II | X |
| 2438 | cg24150623 | MIR125B1; LOC39995 | TSS1500; Body | | TRUE | I | 11 |
| | | 9 | | | | | |
| 2439 | cg24161501 | TCTEXID1 | 5′UTR | Island | TRUE | II | 1 |
| 2440 | cg24211504 | SIM1 | Body | Island | TRUE | I | 6 |
| 2441 | cg24221648 | | | Island | TRUE | I | 13 |
| 2442 | cg24233426 | | | | NA | II | 7 |
| 2443 | cg24252695 | | | Island | NA | II | 1 |
| 2444 | cg24258699 | MSC | TSS200 | S_Shore | NA | I | 8 |
| 2445 | cg24279418 | | | N_Shore | NA | I | 3 |
| 2446 | cg24281267 | TULP1 | Body | Island | TRUE | I | 6 |
| 2447 | cg24302235 | SLC2A2 | TSS1500 | Island | TRUE | II | 3 |
| 2448 | cg24306340 | CACHD1 | Body | S_Shore | NA | II | 1 |
| 2449 | cg24315973 | GSTCD; GSTCD | TSS200; 5′UTR | S_Shore | NA | II | 4 |
| 2450 | cg24326574 | UBL4A | Body | N_Shore | TRUE | II | X |
| 2451 | cg24327132 | PKM2; PKM2; PKM2 | 5′UTR; 5′UTR; 5′UT | N_Shore | NA | II | 15 |
| | | | R | | | | |
| 2452 | cg24331301 | CDH23; CDH23 | TSS1500; TSS1500 | Island | NA | I | 10 |
| 2453 | cg24335070 | | | | TRUE | II | 2 |
| 2454 | cg24338491 | | | | TRUE | II | 12 |
| 2455 | cg24346905 | NTNG1; NTNG1; NTN | Body; Body; Body | Island | NA | I | 1 |
| | | G1 | | | | | |
| 2456 | cg24350771 | | | | NA | II | 7 |
| 2457 | cg24383507 | KCNIP2; KCNIP2; KC | Body; Body; TSS15 | N_Shelf | TRUE | I | 10 |
| | | NIP2; KCNIP2; KCNIP | 00; Body; Body; Bod | | | | |
| | | 2; KCNIP2; KCNIP2 | y; Body | | | | |
| 2458 | cg24384051 | OR8B3 | 1stExon | | NA | II | 11 |
| 2459 | cg24396429 | SLC8A1 | TSS200 | | TRUE | II | 2 |
| 2460 | cg24401557 | FGF13; FGF13; FGF13; | Body; TSS200; Bod | Island | NA | I | X |
| | | FGF13; FGF13; FGF13 | y; Body; Body; Body | | | | |
| 2461 | cg24410453 | C6orf142 | Body | | TRUE | II | 6 |
| 2462 | cg24415208 | CACNG8 | 1stExon | Island | NA | I | 19 |
| 2463 | cg24427271 | USP36 | 5′UTR | N_Shore | NA | II | 17 |
| 2464 | cg24439334 | PEX10; PEX10 | TSS1500; TSS1500 | N_Shore | NA | I | 1 |
| 2465 | cg24448259 | CPVL; CPVL; CPVL; C | 5′UTR; 1stExon; 1st | | TRUE | II | 7 |
| | | PVL | Exon; 5′UTR | | | | |
| 2466 | cg24452260 | GRIA2; GRIA2; GRIA2 | Body; Body; Body | Island | NA | II | 4 |
| 2467 | cg24472375 | | | N_Shore | NA | II | 6 |
| 2468 | cg24472939 | | | S_Shelf | NA | II | 4 |
| 2469 | cg24484138 | C20orf112 | 5′UTR | Island | NA | II | 20 |
| 2470 | cg24504927 | | | | TRUE | I | 12 |
| 2471 | cg24507144 | FGF14 | Body | Island | NA | II | 13 |
| 2472 | cg24508426 | | | Island | NA | I | 10 |
| 2473 | cg24509810 | CTNNA2; CTNNA2 | Body; Body | Island | NA | II | 2 |
| 2474 | cg24512400 | KLK10; KLK10; KLK1 | 5′UTR; 5′UTR; 5′UT | Island | NA | I | 19 |
| | | 0 | R | | | | |
| 2475 | cg24517686 | | | S_Shore | TRUE | II | 1 |
| 2476 | cg24525457 | C7orf51 | Body | Island | NA | I | 7 |
| 2477 | cg24564492 | | | Island | NA | II | 10 |
| 2478 | cg24569637 | AOX1 | TSS200 | Island | TRUE | I | 2 |
| 2479 | cg24600895 | NLGN1 | 5′UTR | | NA | II | 3 |
| 2480 | cg24601030 | DIXDC1; DIXDC1 | TSS1500; Body | N_Shore | NA | II | 11 |
| 2481 | cg24603941 | MIA2; MIA2 | 5′UTR; 1stExon | | NA | II | 14 |
| 2482 | cg24622143 | RASA3 | Body | Island | NA | II | 13 |
| 2483 | cg24625136 | | | Island | NA | I | 3 |
| 2484 | cg24626659 | C10orf93 | Body | N_Shore | NA | I | 10 |
| 2485 | cg24664038 | | | | NA | II | 6 |
| 2486 | cg24671344 | PDPN; PDPN; PDPN; P | TSS1500; TSS1500 | Island | NA | I | 1 |
| | | DPN | ; Body; Body | | | | |
| 2487 | cg24672833 | | | Island | NA | I | 17 |
| 2488 | cg24686074 | | | Island | NA | I | 10 |
| 2489 | cg24686610 | ZIC4; ZIC1 | TSS1500; TSS1500 | N_Shore | TRUE | I | 3 |
| 2490 | cg24690692 | | | | TRUE | II | 7 |
| 2491 | cg24693341 | PHOX2B | 3′UTR | N_Shore | NA | II | 4 |
| 2492 | cg24701267 | | | | TRUE | II | 3 |
| 2493 | cg24701575 | | | N_Shore | NA | II | 11 |
| 2494 | cg24710320 | MGC2889; BRASLS | TSS1500; TSS200 | Island | NA | I | 3 |
| 2495 | cg24719107 | POSTN; POSTN; POST | 5′UTR; 1stExon; 1st | | NA | II | 13 |
| | | N; POSTN; POSTN; PO | Exon; 1stExon; 5′U | | | | |
| | | STN; POSTN; POSTN | TR; 5′UTR; 1stExon | | | | |
| | | | ; 5′UTR | | | | |
| 2496 | cg24722073 | | | N_Shore | NA | I | 4 |
| 2497 | cg24725574 | HLA-J; NCRNA00171 | Body; Body | Island | TRUE | I | 6 |
| 2498 | cg24725576 | | | N_Shore | NA | II | 14 |
| 2499 | cg24727130 | ELFN1 | 5′UTR | | NA | II | 7 |
| 2500 | cg24727399 | QRFPR | TSS200 | Island | NA | I | 4 |
| 2501 | cg24728018 | CLVS2; CLVS2 | 5′UTR; 1stExon | S_Shore | NA | I | 6 |
| 2502 | cg24734254 | | | | NA | II | 1 |
| 2503 | cg24740026 | | | Island | NA | I | 5 |
| 2504 | cg24740531 | BCL2; BCL2 | Body; Body | Island | NA | I | 18 |
| 2505 | cg24771804 | ONECUT2 | Body | Island | TRUE | II | 18 |
| 2506 | cg24773418 | | | Island | NA | I | 14 |
| 2507 | cg24805875 | FAM162B | TSS200 | Island | NA | II | 6 |
| 2508 | cg24812837 | TLX1 | Body | Island | TRUE | I | 10 |
| 2509 | cg24833737 | | | S_Shore | NA | I | 8 |
| 2510 | cg24843380 | ZNF454 | TSS1500 | Island | NA | I | 5 |
| 2511 | cg24843474 | RGS7 | TSS200 | Island | NA | I | 1 |
| 2512 | cg24848035 | RG522 | TSS200 | Island | TRUE | I | 8 |
| 2513 | cg24852713 | HMHA1 | Body | Island | NA | II | 19 |
| 2514 | cg24853313 | MIR892A | Body | | NA | II | X |
| 2515 | cg24874180 | DKK2 | Body | N_Shore | NA | II | 4 |
| 2516 | cg24881834 | ME1 | TSS1500 | Island | NA | II | 6 |
| 2517 | cg24884142 | TBX15; TBX15 | 1stExon; 5′UTR | Island | TRUE | I | 1 |
| 2518 | cg24884703 | | | Island | NA | I | 1 |
| 2519 | cg24888814 | COPZ1 | Body | | TRUE | II | 12 |
| 2520 | cg24892966 | | | | NA | I | 6 |
| 2521 | cg24915503 | PCDHA7; PCDHA12; P | Body; Body; Body; | N_Shelf | NA | II | 5 |
| | | CDHA6; PCDHA10; P | Body; Body; Body; | | | | |
| | | CDHA4; PCDHA11; P | Body; Body; Body; | | | | |
| | | CDHA8; PCDHA6; PC | Body; Body; Body; | | | | |
| | | DHA1; PCDHA2; PCD | Body; Body; Body; | | | | |
| | | HA1; PCDHA9; PCDH | Body; Body | | | | |
| | | A13; PCDHA5; PCDH | | | | | |
| | | AC1; PCDHA3; PCDH | | | | | |
| | | A10 | | | | | |
| 2522 | cg24924958 | NMBR | TSS200 | Island | NA | II | 6 |
| 2523 | cg24928391 | SOX17 | TSS200 | Island | NA | I | 8 |
| 2524 | cg24932585 | | | Island | NA | I | 19 |
| 2525 | cg24939845 | | | Island | NA | I | 1 |
| 2526 | cg24945701 | | | Island | NA | I | 3 |
| 2527 | cg24956533 | LOC145845 | Body | S_Shore | NA | II | 15 |
| 2528 | cg24974737 | | | Island | TRUE | I | 17 |
| 2529 | cg24999518 | NIMH2; NHLH2 | 5′UTR; 5′UTR | Island | TRUE | II | 1 |
| 2530 | cg25029264 | C15orf26 | TSS1500 | N_Shore | NA | II | 15 |
| 2531 | cg25037730 | | | Island | TRUE | II | 14 |
| 2532 | cg25060829 | ZSCAN12; ZSCAN12 | TSS200; TSS200 | Island | NA | I | 6 |
| 2533 | cg25072733 | C13orf36 | 5′UTR | S_Shore | NA | I | 13 |
| 2534 | cg25113237 | | | Island | NA | I | 5 |
| 2535 | cg25119415 | MNDA | TSS1500 | | NA | II | 1 |
| 2536 | cg25124739 | EPAS1 | Body | Island | NA | II | 2 |
| 2537 | cg25129124 | KBTBD5 | 1stExon | Island | NA | I | 3 |
| 2538 | cg25156485 | MSN | Body | Island | NA | I | X |
| 2539 | cg25178784 | C11orf2 | Body | Island | NA | I | 11 |
| 2540 | cg25203962 | | | Island | TRUE | II | 3 |
| 2541 | cg25219333 | ARFIP2; FXC1 | Body; TSS1500 | N_Shore | NA | II | 11 |
| 2542 | cg25228422 | | | Island | NA | I | 7 |
| 2543 | cg25230235 | POLR2I; TBCB | Body; TSS1500 | Island | NA | II | 19 |
| 2544 | cg25236484 | | | Island | NA | I | X |
| 2545 | cg25243455 | PAX8; PAX8; PAX8; P | Body; Body; Body; | Island | NA | I | 2 |
| | | AX8; PAX8; LOC4408 | Body; Body; Body | | | | |
| | | 39 | | | | | |
| 2546 | cg25266629 | TLX1 | Body | Island | TRUE | I | 10 |
| 2547 | cg25271525 | | | | NA | II | 15 |
| 2548 | cg25285904 | | | Island | NA | I | 18 |
| 2549 | cg25308354 | | | Island | NA | I | 8 |
| 2550 | cg25340966 | TBX15 | TSS200 | Island | TRUE | I | 1 |
| 2551 | cg25363445 | ALX4 | Body | Island | TRUE | II | 11 |
| 2552 | cg25368103 | | | | TRUE | II | 8 |
| 2553 | cg25374649 | C18orf1; C18orf1; C18o | TSS1500; Body; Bo | | TRUE | II | 18 |
| | | rf1; C18orf1 | dy; TSS1500 | | | | |
| 2554 | cg25375162 | | | | TRUE | II | 3 |
| 2555 | cg25375916 | SLC33A1 | Body | N_Shelf | TRUE | II | 3 |
| 2556 | cg25387636 | SLC47A1 | TSS1500 | Island | NA | I | 17 |
| 2557 | cg25407876 | DNAJC9 | Body | N_Shore | NA | II | 10 |
| 2558 | cg25408008 | MIER1; WDR78; MIER | 5′UTR; TSS1500; 5′ | S_Shore | NA | II | 1 |
| | | 1; MIER1; MIER1; MIE | UTR; 5′UTR; 5′UTR | | | | |
| | | R1; MIER1; MIER1; W | ; Body; 5′UTR; Body | | | | |
| | | DR78; MIER1 | ; TSS1500; 5′UTR | | | | |
| 2559 | cg25416067 | SEPT9; SEPT9; SEPT9; | 5′UTR; 5′UTR; Bod | | TRUE | I | 17 |
| | | SEPT9 | y; 1stExon | | | | |
| 2560 | cg25456959 | ASB12 | TSS1500 | | NA | II | X |
| 2561 | cg25469406 | | | | NA | II | 11 |
| 2562 | cg25489866 | FAM81A | TSS1500 | N_Shore | NA | I | 15 |
| 2563 | cg25500080 | PCDHAC2; PCDHAC2 | 1stExon; TSS200; B | | | | |
| | | ; PCDHA7; PCDHA12; | ody; Body; Body; Bo | | | | |
| | | PCDHA6; PCDHA10; P | dy; Body; Body; Bod | | | | |
| | | CDHA4; PCDHA11; P | y; Body; Body; Body | | | | |
| | | CDHA8; PCDHA6; PC | ; Body; Body; Body; | | | | |
| | | DHA1; PCDHA2; PCD | Body; Body; Body; 5 | | | | |
| | | HA1; PCDHA9; PCDH | ′UTR; | | | | |
| | | A13; PCDHA5; PCDH | | | | | |
| | | AC1; PCDHA3; PCDH | | | | | |
| | | AC2; PCDHA10 | | | | | |
| Body | Island | NA | 1 | 5 | 140346199 | TGGGGAGT | |
| | | | | | | CAGCGAGG | |
| | | | | | | ACGGTGGG | |
| | | | | | | GCCAGGAG | |
| | | | | | | CCCTTGGG | |
| | | | | | | AGGGCCTA | |
| | | | | | | CG | |
| 2564 | cg25510164 | GBA3; GBA3 | Body; Body | | NA | II | 4 |
| 2565 | cg25510609 | AKAP13; AKAP13; AK | Body; Body; Body | | TRUE | I | 15 |
| | | AP13 | | | | | |
| 2566 | cg25514273 | KLHL1; ATXN8OS | 1stExon; Body | Island | TRUE | I | 13 |
| 2567 | cg25520146 | HPYR1 | TSS1500 | | NA | II | 8 |
| 2568 | cg25528646 | GABRE | TSS200 | Island | NA | I | X |
| 2569 | cg25556905 | ADCY4 | TSS200 | Island | NA | I | 14 |
| 2570 | cg25565383 | RAD51AP2 | TSS200 | | NA | II | 2 |
| 2571 | cg25566285 | PTPRN2; PTPRN2; PT | Body; Body; Body | S_Shelf | NA | I | 7 |
| | | PRN2 | | | | | |
| 2572 | cg25588787 | | | Island | TRUE | II | 5 |
| 2573 | cg25594119 | | | N_Shore | NA | I | 18 |
| 2574 | cg25597760 | | | Island | NA | I | 17 |
| 2575 | cg25599924 | ACOX3; ACOX3 | Body; Body | | NA | II | 4 |
| 2576 | cg25602651 | DCAF8L1 | TSS200 | | NA | II | X |
| 2577 | cg25608973 | C2orf43 | 5′UTR | N_Shore | NA | II | 2 |
| 2578 | cg25610702 | CRIP3 | TSS1500 | S_Shore | TRUE | I | 6 |
| 2579 | cg25645064 | | | | TRUE | I | 3 |
| 2580 | cg25648436 | ZSCAN1 | Body | Island | NA | I | 19 |
| 2581 | cg25662926 | ZFHX4; LOC10019237 | 5′UTR; Body | S_Shore | NA | I | 8 |
| | | 8 | | | | | |
| 2582 | cg25663755 | VILL | Body | N_Shore | NA | II | 3 |
| 2583 | cg25690715 | SEPT9; SEPT9; SEPT9 | 5′UTR; TSS200; Bo | | TRUE | II | 17 |
| | | | dy | | | | |
| 2584 | cg25691167 | FERD3L | 1stExon | Island | TRUE | I | 7 |
| 2585 | cg25705558 | ISLR2; LOC283731; IS | 5′UTR; TSS1500; T | Island | NA | II | 15 |
| | | LR2; ISLR2; ISLR2; ISL | 551500; TSS1500; 1 | | | | |
| | | R2 | stExon; TSS200 | | | | |
| 2586 | cg25708328 | | | N_Shelf | NA | II | 2 |
| 2587 | cg25726664 | GLI3 | TSS1500 | Island | NA | I | 7 |
| 2588 | cg25729795 | FLJ30058 | Body | Island | NA | II | X |
| 2589 | cg25731337 | MAGEA5 | TSS200 | | NA | II | X |
| 2590 | cg25734928 | | | N_Shelf | NA | II | 6 |
| 2591 | cg25741487 | | | Island | NA | I | 3 |
| 2592 | cg25755283 | TBX15 | 5′UTR | N_Shore | NA | II | 1 |
| 2593 | cg25760325 | TR1ML1 | TSS1500 | | NA | II | 4 |
| 2594 | cg25763036 | | | N_Shelf | NA | I | 4 |
| 2595 | cg25773917 | GPR183; UBAC2; UBA | 3′UTR; Body; Body; | | NA | II | 13 |
| | | C2; UBAC2 | Body | | | | |
| 2596 | cg25775494 | NR2E1 | Body | N_Shore | TRUE | I | 6 |
| 2597 | cg25782847 | C11orf88; C11orf88 | TSS200; TSS200 | Island | NA | I | 11 |
| 2598 | cg25800765 | ALX1 | TSS1500 | N_Shore | NA | II | 12 |
| 2599 | cg25810857 | 9-Sep | Body | S_Shore | NA | II | 17 |
| 2600 | cg25830182 | NKX6-1 | Body | N_Shore | NA | II | 4 |
| 2601 | cg25834768 | CAMKK1; CAMKK1; | 5′UTR; 5′UTR; 5′UT | Island | TRUE | I | 17 |
| | | CAMKK1 | R | | | | |
| 2602 | cg25849196 | | | N_Shore | TRUE | I | 2 |
| 2603 | cg25859468 | MEOX2; MEOX2 | 1stExon; 5′UTR | | NA | I | 7 |
| 2604 | cg25872852 | RPL 2; SNORA52 | Body; TSS200 | S_Shore | NA | II | 11 |
| 2605 | cg25884711 | NPY; NPY | 5′UTR; 1stExon | Island | NA | I | 7 |
| 2606 | cg25887010 | | | Island | NA | II | X |
| 2607 | cg25896644 | | | Island | TRUE | I | 11 |
| 2608 | cg25913520 | | | N_Shore | TRUE | I | 4 |
| 2609 | cg25923056 | EGLN2; EGLN2; EGL | 1stExon; 5′UTR; 5′U | N_Shore | NA | I | 19 |
| | | N2 | TR | | | | |
| 2610 | cg25928819 | | | N_Shore | NA | I | 12 |
| 2611 | cg25947619 | AKAP13; AKAP13; AK | Body; Body; Body | | TRUE | I | 15 |
| | | AP13 | | | | | |
| 2612 | cg25951981 | GABRA4 | TSS200 | Island | NA | I | 4 |
| 2613 | cg25961816 | | | | TRUE | II | 6 |
| 2614 | cg25971727 | ACSS3 | TSS1500 | N_Shore | NA | I | 12 |
| 2615 | cg25977026 | KDM1A; KDM1A | Body; Body | S_Shore | NA | II | 1 |
| 2616 | cg25996119 | NKX2-1 | TSS1500 | Island | NA | I | 14 |
| 2617 | cg26000619 | AMH | Body | Island | NA | I | 19 |
| 2618 | cg26043257 | CD38 | 1stExon | Island | TRUE | II | 4 |
| 2619 | cg26043322 | OR10J5 | TSS1500 | | NA | II | 1 |
| 2620 | cg26053697 | COL4A2 | Body | S_Shore | NA | I | 13 |
| 2621 | cg26104752 | TBX15 | 5′UTR | S_Shore | NA | II | 1 |
| 2622 | cg26127662 | HOXA3; HOXA3; HO | 5′UTR; TSS1500; 5′ | N_Shore | NA | II | 7 |
| | | XA3 | UTR | | | | |
| 2623 | cg26132320 | MMP9 | Body | Island | NA | I | 20 |
| 2624 | cg26151163 | ILKAP | 3′UTR | | NA | II | 2 |
| 2625 | cg26158950 | FLJ22536 | Body | S_Shore | NA | II | 6 |
| 2626 | cg26165269 | | | Island | NA | II | 17 |
| 2627 | cg26221992 | | | Island | NA | II | 6 |
| 2628 | cg26222247 | ANPEP | 5′UTR | Island | NA | II | 15 |
| 2629 | cg26232247 | RIN2 | Body | | TRUE | I | 20 |
| 2630 | cg26237681 | SYCP2L; SYCP2L | 1stExon; 5′UTR | Island | NA | II | 6 |
| 2631 | cg26238975 | MIR134; MIR668; MIR | TSS1500; TSS1500 | | NA | I | 14 |
| | | 382; MIR485 | ; Body; TSS1500 | | | | |
| 2632 | cg26246880 | PYHIN1; PYHIN1; PY | TSS1500; TSS1500 | | NA | II | 1 |
| | | HIN1; PYHIN1 | ; TSS1500; TSS150 | | | | |
| | | | 0 | | | | |
| 2633 | cg26251098 | NTM | Body | | TRUE | II | 11 |
| 2634 | cg26253438 | ATP2B4; ATP2B4 | 5′UTR; 5′UTR | Island | TRUE | I | 1 |
| 2635 | cg26261502 | | | N_Shore | NA | II | 11 |
| 2636 | cg26262482 | CACHD1 | Body | S_Shore | NA | II | 1 |
| 2637 | cg26263234 | LRRC3B | 5′UTR | Island | NA | I | 3 |
| 2638 | cg26272220 | ACSS3; ACSS3 | 1stExon; 5′UTR | Island | NA | I | 12 |
| 2639 | cg26277556 | | | | TRUE | II | 14 |
| 2640 | cg26282792 | ZSCAN1 | Body | Island | NA | I | 19 |
| 2641 | cg26296488 | DRD5 | TSS200 | Island | NA | I | 4 |
| 2642 | cg26299756 | | | N_Shelf | NA | II | 6 |
| 2643 | cg26310256 | EGFL6; EGFL6 | Body; Body | S_Shore | TRUE | II | X |
| 2644 | cg26335602 | ZNF389 | 5′UTR | | TRUE | II | 6 |
| 2645 | cg26345888 | DAB1 | TSS200 | S_Shore | NA | I | 1 |
| 2646 | cg26354128 | KIAA1409 | 5′UTR | Island | NA | I | 14 |
| 2647 | cg26359388 | LOC286467 | Body | N_Shelf | NA | II | X |
| 2648 | cg26363759 | SPSB1 | 5′UTR | | TRUE | I | 1 |
| 2649 | cg26383972 | FITR2C | TSS1500 | N_Shore | NA | I | X |
| 2650 | cg26385743 | IMP4 | Body | S_Shore | NA | II | 2 |
| 2651 | cg26394257 | ADAM32 | TSS200 | N_Shore | NA | II | 8 |
| 2652 | cg26396492 | RIN2 | Body | | TRUE | II | 20 |
| 2653 | cg26398467 | | | | TRUE | I | 3 |
| 2654 | cg26398921 | | | Island | NA | II | 1 |
| 2655 | cg26404669 | CCDC65 | TSS200 | | TRUE | I | 12 |
| 2656 | cg26419656 | KLF12 | 5′UTR | N_Shore | NA | I | 13 |
| 2657 | cg26420013 | NSUN2; SRD5A1 | Body; TSS1500 | N_Shore | NA | II | 5 |
| 2658 | cg26432539 | SPATA8 | 1stExon | | NA | II | 15 |
| 2659 | cg26434983 | | | Island | NA | I | 15 |
| 2660 | cg26444528 | PCDH17 | 1stExon | Island | NA | I | 13 |
| 2661 | cg26447413 | GAS1 | 1stExon | Island | NA | II | 9 |
| 2662 | cg26449787 | EYA4; EYA4; EYA4 | TSS1500; TSS1500 | Island | NA | II | 6 |
| | | | ; TSS1500 | | | | |
| 2663 | cg26459372 | ADCY1 | TSS1500 | Island | TRUE | I | 7 |
| 2664 | cg26465602 | | | Island | TRUE | I | 16 |
| 2665 | cg26466587 | MCHR2; MCHR2 | TSS200; TSS200 | Island | TRUE | I | 6 |
| 2666 | cg26499055 | SIM1 | Body | Island | NA | I | 6 |
| 2667 | cg26503653 | DOCK2 | TSS1500 | N_Shore | NA | II | 5 |
| 2668 | cg26517171 | TLX3 | TSS1500 | Island | NA | I | 5 |
| 2669 | cg26530061 | | | S_Shore | NA | II | 7 |
| 2670 | cg26530341 | TNFRSF10A | TSS1500 | S_Shore | NA | II | 8 |
| 2671 | cg26530706 | DTNA; DTNA; DTNA; | TSS200; 5′UTR; 5′U | | TRUE | II | 18 |
| | | DTNA | TR; 5′UTR | | | | |
| 2672 | cg26560222 | DMRTA2; DMRTA2 | 1stExon; 5′UTR | Island | NA | I | 1 |
| 2673 | cg26560414 | CMTM3; CMTM3; CM | 5′UTR; 5′UTR; 5′UT | Island | NA | I | 16 |
| | | TM3; CMTM3; CMTM | R; 1stExon; TSS200 | | | | |
| | | 3 | | | | | |
| 2674 | cg26561986 | USP44; USP44 | 5′UTR; 5′UTR | N_Shelf | NA | II | 12 |
| 2675 | cg26567423 | FLJ12825 | Body | Island | NA | I | 12 |
| 2676 | cg26573704 | ADAMTS12 | TSS200 | Island | NA | II | 5 |
| 2677 | cg26583474 | RPS7 | Body | S_Shore | NA | II | 2 |
| 2678 | cg26596964 | | | Island | NA | I | 10 |
| 2679 | cg26597539 | PTGER4 | 5′UTR | Island | NA | II | 5 |
| 2680 | cg26603632 | | | N_Shore | NA | I | 17 |
| 2681 | cg26605683 | STC2 | Body | N_Shelf | TRUE | I | 5 |
| 2682 | cg26615127 | DNAJC6 | Body | Island | TRUE | I | 1 |
| 2683 | cg26618965 | | | Island | TRUE | I | 8 |
| 2684 | cg26623444 | | | Island | TRUE | I | 7 |
| 2685 | cg26625629 | C2orf43 | TSS200 | Island | NA | I | 2 |
| 2686 | cg26628435 | BEX1; BEX1 | 5′UTR; 1stExon | Island | NA | I | X |
| 2687 | cg26631984 | | | N_Shore | NA | I | 2 |
| 2688 | cg26650638 | | | Island | NA | I | 16 |
| 2689 | cg26677406 | SLCO3A1; SLCO3A1 | Body; Body | | TRUE | II | 15 |
| 2690 | cg26678970 | | | S_Shore | NA | I | 13 |
| 2691 | cg26679855 | | | N_Shelf | NA | II | 3 |
| 2692 | cg26702958 | FLJ41350 | Body | Island | TRUE | I | 10 |
| 2693 | cg26708220 | MEIS2; MEIS2; MEIS2 | Body; Body; Body; | Island | NA | I | 15 |
| | | ; MEIS2; MEIS2; MEIS | Body; Body Body; | | | | |
| | | 2; MEIS2 | Body | | | | |
| 2694 | cg26717763 | | | S_Shore | NA | I | 6 |
| 2695 | cg26751645 | RBPJL | Body | Island | NA | I | 20 |
| 2696 | cg26755141 | SIM1 | Body | N_Shore | TRUE | II | 6 |
| 2697 | cg26764761 | JPH3 | Body | S_Shelf | NA | I | 16 |
| 2698 | cg26767897 | XDH | Body | | NA | II | 2 |
| 2699 | cg26769720 | | | Island | NA | I | 7 |
| 2700 | cg26772854 | | | Island | TRUE | II | 3 |
| 2701 | cg26796095 | SAFB | Body | S_Shore | NA | II | 19 |
| 2702 | cg26799474 | CASP8; CASP8; CASP | 5′UTR; 5′UTR; 5′UT | | NA | II | 2 |
| | | 8; CASP8 | R; 5′UTR | | | | |
| 2703 | cg26814276 | GALNTL6 | TSS1500 | Island | NA | I | 4 |
| 2704 | cg26822986 | ANKRD29 | Body | Island | NA | II | 18 |
| 2705 | cg26827394 | UBE2I; UBE2I; UBE2I; | 5′UTR; 5′UTR; 5′UT | N_Shore | TRUE | I | 16 |
| | | UBE2I | R; 5′UTR | | | | |
| 2706 | cg26835675 | SHISA9 | Body | | TRUE | II | 16 |
| 2707 | cg26836696 | | | N_Shore | NA | I | 5 |
| 2708 | cg26837708 | YBX1 | Body | S_Shore | NA | II | 1 |
| 2709 | cg26848718 | WT1; WT1; WT1; WT1 | Body; Body; Body; | Island | NA | I | 11 |
| | | | Body | | | | |
| 2710 | cg26855689 | HGS | 3′UTR | N_Shore | NA | I | 17 |
| 2711 | cg26870192 | SLC35D3 | TSS1500 | N_Shore | NA | I | 6 |
| 2712 | cg26870638 | ZXDC; ZXDC | Body; Body | N_Shore | TRUE | II | 3 |
| 2713 | cg26874872 | | | | TRUE | II | 16 |
| 2714 | cg26891410 | TAL1 | TSS1500 | N_Shore | NA | II | 1 |
| 2715 | cg26896572 | C3orf14 | TSS1500 | Island | NA | II | 3 |
| 2716 | cg26917140 | | | N_Shore | TRUE | II | 1 |
| 2717 | cg26926076 | LAMA2; LAMA2 | Body; Body | | TRUE | I | 6 |
| 2718 | cg26926612 | | | Island | NA | I | 1 |
| 2719 | cg26929261 | ARL10 | TSS1500 | N_Shore | NA | I | 5 |
| 2720 | cg26937500 | CARD11; CARD11 | 1stExon; 5′UTR | | TRUE | I | 7 |
| 2721 | cg26954695 | MZF1; MGC2752; LOC | TSS1500; TSS1500 | N_Shore | NA | I | 19 |
| | | 100131691; MZF1 | ; Body; TSS1500 | | | | |
| 2722 | cg26954968 | SIM1 | Body | Island | TRUE | I | 6 |
| 2723 | cg26966124 | SIMI | TSS1500 | Island | TRUE | II | 6 |
| 2724 | cg26979473 | PTN; PTN | 5′UTR; 1stExon | | NA | II | 7 |
| 2725 | cg26983177 | | | Island | TRUE | I | 19 |
| 2726 | cg26992010 | | | N_Shore | TRUE | II | 15 |
| 2727 | cg26993198 | | | S_Shore | NA | I | 5 |
| 2728 | cg26996175 | | | Island | NA | II | 11 |
| 2729 | cg27014380 | | | Island | NA | I | 3 |
| 2730 | cg27032232 | DPP6; DPP6 | 1stExon; 5′UTR | Island | NA | I | 7 |
| 2731 | cg27036111 | SLC6A5 | TSS1500 | S_Shore | NA | II | 11 |
| 2732 | cg27039467 | HN1L | Body | S_Shore | NA | II | 16 |
| 2733 | cg27039662 | | | Island | TRUE | I | 8 |
| 2734 | cg27062369 | DDIT4L | 5′UTR | Island | NA | I | 4 |
| 2735 | cg27071152 | LOC646999 | Body | Island | TRUE | I | 7 |
| 2736 | cg27080287 | | | Island | NA | I | X |
| 2737 | cg27088150 | | | N_Shore | NA | I | 3 |
| 2738 | cg27096043 | ATP11A; ATP11A | Body; Body | S_Shore | TRUE | II | 13 |
| 2739 | cg27109600 | FLJ32063 | TSS1500 | N_Shore | NA | I | 2 |
| 2740 | cg27112666 | | | N_Shore | NA | I | 6 |
| 2741 | cg27116888 | SALL3 | Body | | TRUE | I | 18 |
| 2742 | cg27120816 | CDH8 | TSS200 | Island | NA | I | 16 |
| 2743 | cg27127608 | EBF1 | Body | | TRUE | II | 5 |
| 2744 | cg27134730 | | | | TRUE | II | 7 |
| 2745 | cg27137084 | TMEM132C | Body | | TRUE | II | 12 |
| 2746 | cg27138281 | ME1 | TSS200 | Island | NA | I | 6 |
| 2747 | cg27140058 | BANK1; BANK1 | Body; Body | S_Shore | NA | I | 4 |
| 2748 | cg27153155 | SIM1 | Body | N_Shore | NA | II | 6 |
| 2749 | cg27198013 | RAI1 | 3′UTR | Island | TRUE | I | 17 |
| 2750 | cg27203895 | NTM | Body | | TRUE | II | 11 |
| 2751 | cg27225220 | LHX9 | Body | S_Shore | NA | II | 1 |
| 2752 | cg27228712 | | | Island | NA | II | 7 |
| 2753 | cg27252467 | LOC348021 | Body | | NA | II | 13 |
| 2754 | cg27252696 | SIM1 | TSS1500 | Island | NA | I | 6 |
| 2755 | cg27262412 | TBX15 | 5′UTR | Island | TRUE | II | 1 |
| 2756 | cg27268556 | | | N_Shore | NA | II | 1 |
| 2757 | cg27272235 | | | Island | TRUE | II | 1 |
| 2758 | cg27292588 | PTCH2 | 3′UTR | | TRUE | II | 1 |
| 2759 | cg27295832 | ANKRD34C | TSS1500 | Island | TRUE | II | 15 |
| 2760 | cg27297576 | | | N_Shore | NA | I | 12 |
| 2761 | cg27303421 | | | | TRUE | II | 6 |
| 2762 | cg27350602 | | | Island | NA | I | X |
| 2763 | cg27376707 | A2BP1; A2BP1 | 5′UTR; 5′UTR | Island | NA | I | 16 |
| 2764 | cg27401846 | | | N_Shore | TRUE | II | 11 |
| 2765 | cg27404023 | | | Island | TRUE | I | 2 |
| 2766 | cg27406678 | NKX6-1 | Body | N_Shore | NA | II | 4 |
| 2768 | cg27422141 | PCDH9; PCDH9 | 5′UTR; 5′UTR | N_Shore | NA | II | 13 |
| 2769 | cg27424370 | | | N_Shore | NA | II | X |
| 2770 | cg27425314 | DHDDS; HMGN2; DH | 3′UTR; TSS1500; 3′ | N_Shore | TRUE | I | 1 |
| | | DDS | UTR | | | | |
| 2771 | cg27452341 | CLCN1 | Body | Island | TRUE | II | 7 |
| 2772 | cg27493010 | | | Island | NA | II | 12 |
| 2773 | cg27510539 | VIPR1 | TSS1500 | Island | NA | II | 3 |
| 2774 | cg27522060 | RASL12; RASL12 | 5′UTR; 1stExon | Island | NA | I | 15 |
| 2775 | cg27527108 | | | Island | TRUE | I | 1 |
| 2776 | cg27528510 | EMID2 | TSS200 | Island | NA | I | 7 |
| 2777 | cg27531496 | | | | NA | II | 1 |
| 2778 | cg27541541 | PRL; PRL | TSS1500; 5′UTR | | NA | II | 6 |
| 2779 | cg27552679 | ISLR2; ISLR2 SLR2; I | Body; Body; Body; | Island | NA | I | 15 |
| | | SLR2 | Body | | | | |
| 2780 | cg27555582 | | | Island | NA | I | 12 |
| 2781 | cg27559661 | CPNE2 | TSS1500 | N_Shore | NA | I | 16 |
| 2782 | cg27564792 | FEZF2 | TSS1500 | S_Shore | NA | II | 3 |
| 2783 | cg27570984 | | | | NA | I | 16 |
| 2784 | cg27590397 | SSTR1; SSTR1 | 1stExon; 5′UTR | Island | NA | II | 14 |
| 2785 | cg27603915 | NELL1; NELL1 | Body; Body | S_Shore | NA | I | 11 |
| 2786 | cg27615938 | NCRNA00188; NCRN | Body; Body; Body; | S_Shore | NA | II | 17 |
| | | A00188; NCRNA0018 | Body; Body; Body; | | | | |
| | | 8; NCRNA00188; NCR | Body; Body; Body; | | | | |
| | | NA00188; NCRNA001 | Body; Body; Body; | | | | |
| | | 88; NCRNA00188; NC | Body; Body; Body; | | | | |
| | | RNA00188; NCRNA00 | Body; Body; Body; | | | | |
| | | 188; NCRNA00188; NC | Body; Body; Body; | | | | |
| | | RNA00188; NCRNA00 | Body; Body | | | | |
| | | 188; NCRNA00188; | | | | | |
| | | NCRNA00188; NCRN | | | | | |
| | | A00188; NCRNA0018 | | | | | |
| | | 8; NCRNA00188; NCR | | | | | |
| | | NA00188; SNORD65; | | | | | |
| | | NCRNA00188; NCRN | | | | | |
| | | A00188; NCRNA0018 | | | | | |
| | | 8; NCRNA00188 | | | | | |
| 2787 | cg27635135 | | | | NA | II | 7 |
| 2788 | cg27638126 | MEOX2 | Body | | NA | II | 7 |
| 2789 | cg27663389 | | | Island | NA | I | 19 |
| 2789 | cg03456213 | C9orf3 | NM_032823 | Body | chr9: 9776664 | Island | |
| | | | | | 9-97767955 | | |
| 2789 | cg18315943 | | | | chr5: 3188099 | Island | |
| | | | | | -3188375 | | |
| 2789 | cg12737160 | COL4A6; COL4A6; CO | NM_033641; NM_ | 1stExon; Body; 5′U | | | |
| | | L4A6 | 001847; NM_03364 | TR | | | |
| | | | 1 | | | | |
| 2789 | cg12098872 | | | | chr19: 477427 | Island | |
| | | | | | 25-47743151 | | |
| 2789 | cg18423852 | KCNH2; KCNH2; KCN | NM_172057; NM_ | TSS200; Body; Bod | chr7: 1506528 | Island | |
| | | H2 | 172056; NM_00023 | y | 07- | | |
| | | | 8 | | 150653080 | | |
| 2789 | cg09771468 | BARHL1 | NM_020064 | Body | chr9: 1354619 | Island | |
| | | | | | 34- | | |
| | | | | | 135462909 | | |
| 2789 | cg05733231 | | NR_024271 | Body | chr7: 4580818 | N_Shore | |
| | | | | | 3-45808745 | | |
| 2789 | cg04145287 | | | | chr1: 2655151 | Island | |
| | | | | | 7-26552194 | | |
| 2748 | cg27153155 | SIM1 | Body | N_Shore | NA | II | 6 |
| 2749 | cg27198013 | RAI1 | 3′UTR | Island | TRUE | I | 17 |
| 2750 | cg27203895 | NTM | Body | | TRUE | II | 11 |
| 2751 | cg27225220 | LHX9 | Body | S_Shore | NA | II | 1 |
| 2752 | cg27228712 | | | Island | NA | II | 7 |
| 2753 | cg27252467 | LOC348021 | Body | | NA | II | 13 |
| 2754 | cg27252696 | SIM1 | TSS1500 | Island | NA | I | 6 |
| 2755 | cg27262412 | TBX15 | 5′UTR | Island | TRUE | II | 1 |
| 2756 | cg27268556 | | | N_Shore | NA | II | 1 |
| 2757 | cg27272235 | | | Island | TRUE | II | 1 |
| 2758 | cg27292588 | PTCH2 | 3′UTR | | TRUE | II | 1 |
| 2759 | cg27295832 | ANKRD34C | TSS1500 | Island | TRUE | II | 15 |
| 2760 | cg27297576 | | | N_Shore | NA | I | 12 |
| 2761 | cg27303421 | | | | TRUE | II | 6 |
| 2762 | cg27350602 | | | Island | NA | I | X |
| 2763 | cg27376707 | A2BP1; A2BP1 | 5′UTR; 5′UTR | Island | NA | I | 16 |
| 2764 | cg27401846 | | | N_Shore | TRUE | II | 11 |
| 2765 | cg27404023 | | | Island | TRUE | I | 2 |
| 2766 | cg27406678 | NKX6-1 | Body | N_Shore | NA | II | 4 |
| 2767 | cg27418402 | PSMD6 | Body | N_Shore | NA | II | 3 |
| 2768 | cg27422141 | PCDH9; PCDH9 | 5′UTR; 5′UTR | N_Shore | NA | II | 13 |
| 2769 | cg27424370 | | | N_Shore | NA | II | X |
| 2770 | cg274253I4 | DHDDS; HMGN2; DH | 3′UTR; TSS1500; 3′ | N_Shore | TRUE | I | 1 |
| | | DDS | UTR | | | | |
| 2771 | cg27452341 | CLCN1 | Body | Island | TRUE | II | 7 |
| 2772 | cg27493010 | | | Island | NA | II | 12 |
| 2773 | cg27510539 | VIPR1 | TSS1500 | Island | NA | II | 3 |
| 2774 | cg27522060 | RASL12; RASL12 | 5′UTR; 1stExon | Island | NA | I | 15 |
| 2775 | cg27527108 | | | Island | TRUE | I | 1 |
| 2776 | cg27528510 | EMID2 | TSS200 | Island | NA | I | 7 |
| 2777 | cg27531496 | | | | NA | II | 1 |
| 2778 | cg27541541 | PRL; PRL | TSS1500; 5′UTR | | NA | II | 6 |
| 2779 | cg27552679 | ISLR2; ISLR2 SLR2; I | Body; Body; Body; | Island | NA | I | 15 |
| | | SLR2 | Body | | | | |
| 2780 | cg27555582 | | | Island | NA | I | 12 |
| 2781 | cg27559661 | CPNE2 | TSS1500 | N_Shore | NA | I | 16 |
| 2782 | cg27564792 | FEZF2 | TSS1500 | S_Shore | NA | II | 3 |
| 2783 | cg27570984 | | | | NA | I | 16 |
| 2784 | cg27590397 | SSTR1; SSTR1 | 1stExon; 5′UTR | Island | NA | II | 14 |
| 2785 | cg27603915 | NELL1; NELL1 | Body; Body | S_Shore | NA | I | 11 |
| 2786 | cg27615938 | NCRNA00188; NCRN | Body; Body; Body; | S_Shore | NA | II | 17 |
| | | A00188; NCRNA0018 | Body; Body; Body; | | | | |
| | | 8; NCRNA00188; NCR | Body; Body; Body; | | | | |
| | | NA00188; NCRNA001 | Body; Body; Body; | | | | |
| | | 88; NCRNA00188; NC | Body; Body; Body; | | | | |
| | | RNA00188; NCRNA00 | Body; Body; Body; | | | | |
| | | 188; NCRNA00188; NC | Body; Body; Body; | | | | |
| | | RNA00188; NCRNA00 | Body; Body | | | | |
| | | 188; NCRNA00188; | | | | | |
| | | NCRNA00188; NCRN | | | | | |
| | | A00188; NCRNA0018 | | | | | |
| | | 8; NCRNA00188; NCR | | | | | |
| | | NA00188; SNORD65; | | | | | |
| | | NCRNA00188; NCRN | | | | | |
| | | A00188; NCRNA0018 | | | | | |
| | | 8; NCRNA00188 | | | | | |
| 2787 | cg27635135 | | | | NA | II | 7 |
| 2788 | cg27638126 | MEOX2 | Body | | NA | II | 7 |
| 2789 | cg27663389 | | | Island | NA | I | 19 |
| 2789 | cg03456213 | C9orf3 | NM_032823 | Body | chr9: 9776664 | Island | |
| | | | | | 9-97767955 | | |
| 2789 | cg18315943 | | | | chr5: 3188099 | Island | |
| | | | | | -3188375 | | |
| 2789 | cg12737160 | COL4A6; COL4A6; CO | NM_033641; NM | 1stExon; Body; 5′U | | | |
| | | L4A6 | 001847; NM_03364 | TR | | | |
| | | | 1 | | | | |
| 2789 | cg12098872 | | | | chr19: 477427 | Island | |
| | | | | | 25-47743151 | | |
| 2789 | cg18423852 | KCNH2; KCNH2; KCN | NM_172057; | | | | |
| | | H2 | | | | | |
| NM_1 | TSS200; Bo | chr7:150652807- | Island | | | GAGGGGCT | |
| 72056; | dy; Body | 150653080 | | | | CAGGCTGC | |
| NM_ | | | | | | GGGAGCGC | |
| 000238 | | | | | | CGCTGCAG | |
| | | | | | | GAGCCTGG | |
| | | | | | | GCGGGCTG | |
| | | | | | | CG | |
| 2789 | cg09771468 | BARHL1 | NM_020064 | Body | chr9: 1354619 | Island | |
| | | | | | 34- | | |
| | | | | | 135462909 | | |
| 2789 | cg05733231 | | NR_024271 | Body | chr7: 4580818 | N_Shore | |
| | | | | | 3-45808745 | | |
| 2789 | cg04145287 | | | | chr1: 2655151 | Island | |
| | | | | | 7-26552194 | | |
| 2787 | cg27635135 | | | | NA | II | 7 |
| 2788 | cg27638126 | MEOX2 | Body | | NA | II | 7 |
| 2789 | cg27663389 | | | Island | NA | I | 19 |
| 2789 | cg03456213 | C9orf3 | NM_032823 | Body | chr9: 9776664 | Island | |
| | | | | | 9-97767955 | | |
| 2789 | cg18315943 | | | | chr5: 3188099 | Island | |
| | | | | | -3188375 | | |
| 2789 | cg12737160 | COL4A6; COL4A6; CO | NM_033641; NM_ | 1stExon; Body; 5′U | | | |
| | | L4A6 | 001847; NM_03364 | TR | | | |
| | | | 1 | | | | |
| 2789 | cg12098872 | | | | chr19: 477427 | Island | |
| | | | | | 25-47743151 | | |
| 2789 | cg18423852 | KCNH2; KCNH2; KCN | NM_172057; NM | TSS200; Body; Bod | chr7: 1506528 | Island | |
| | | H2 | 172056; NM_00023 | y | 07- | | |
| | | | 8 | | 150653080 | | |
| 2789 | cg09771468 | BARHL1 | NM_020064 | Body | chr9: 1354619 | Island | |
| | | | | | 34- | | |
| | | | | | 135462909 | | |
| 2789 | cg05733231 | | NR_024271 | Body | chr7: 4580818 | N_Shore | |
| | | | | | 3-45808745 | | |
| 2789 | cg04145287 | | | | chr1: 2655151 | Island | |
| | | | | | 7-26552194 | | |
| | | | | | SEQ ID | MAP | |
| | | | | | NO: | INFO | SourceSeq |
| | | | | | 1 | 14971 | CGGCCGCACCTCACTACCGACCTAAAGATGCGCCTTTGCTAGGCGGCAGC |
| | | | | | | 9536 | |
| | | | | | 2 | 38067 | CTTCCTATTTAGCAGGACCGCAGGGTTGGCAGACAGCAGGGCCTGGGCCG |
| | | | | | | 965 | |
| | | | | | 3 | 21984 | GCCAACCTCGTTTTTGTGGTTTCTGTCTCTTGGTGAGATCTAGGGTAACG |
| | | | | | | 8373 | |
| | | | | | 4 | 26271 | GTAATTCACCAATCACATCGTTGGACTCTTCAGCCAATGGTTTTGTTGCG |
| | | | | | | 718 | |
| | | | | | 5 | 15414 | CCTTTACTACTAAATGCTTCTCCTTTCAAAAGTCAGGGGCATGGGGATCG |
| | | | | | | 6183 | |
| | | | | | 6 | 37123 | GAGGGCACCATGGGAGCTGGCTCTCTTGCTATTCATAAAGCGGGGAGGCG |
| | | | | | | 835 | |
| | | | | | 7 | 22873 | GGGGCGCAGAGCTGGGAGGTCTGGCTCCGCGGGCGGTTGGTGGCGACCCG |
| | | | | | | 6253 | |
| | | | | | 8 | 53075 | CACTGGCACACTGAGGTAATCGAAAAGATCAAACCTATCCTCGTCGATCG |
| | | | | | | 505 | |
| | | | | | 9 | 1018 | GGCCCCTTCTTTATGCAGCCCGTTTGAAATGGTCTCTTGAGCCCAGTGCG |
| | | | | | | 40329 | |
| | | | | | 10 | 10089 | TGCCCACCCACCTGGAGTCCAGAAAGATGAGCTTCATGTCCAGGCTGGCG |
| | | | | | | 6392 | |
| | | | | | 11 | 91185 | TCTCATCGTCTCAGTGGCATTTTTCCTAGGGCTGTTCCGAGAGAACAGCG |
| | | | | | | 168 | |
| | | | | | 12 | 96888 | TGTTAGAAAATTGTGCCATCTACCCGCTATGCTGAGTACTGTCACACTCG |
| | | | | | | 959 | |
| | | | | | 13 | 57261 | CGGGTCCTAAAGGGGAGAGCTGAGCAGCCACTTCAGGGATTGCACTCCGA |
| | | | | | | 886 | |
| | | | | | 14 | 11810 | CGTGCCCAGTTGTACCCTTCCCGCGCAGCGCTCAGGAGCGACAAACCTGG |
| | | | | | | 7540 | |
| | | | | | 15 | 46306 | CGCTGGCTCTGCAGCCCAGCTCAAAAGCCCATCCTCCAGGCCACTTCCCC |
| | | | | | | 111 | |
| | | | | | 16 | 43741 | GAGTGGGGATTCTGGGGCGGGACTCTTTGAAGTCCTAGGTGACCTCTCCG |
| | | | | | | 826 | |
| | | | | | 17 | 50889 | TGTGGCGCCTGCCATTGGCCAGGGGAGGCAGACGGCGCTCTGATTGGCCG |
| | | | | | | 225 | |
| | | | | | 18 | 17267 | TTGGTGTGTGTGTGCAACACAACAATTTGTCAGCTGCTGTTCACAATGCG |
| | | | | | | 1561 | |
| | | | | | 19 | 14711 | CGGGCTGCAGGGGCCTAGCCCAACGCCGCCTCTTCACATTCACCCACCCA |
| | | | | | | 1280 | |
| | | | | | 20 | 14602 | CGGGCCAGTTTCTGGGATCTTGAAGTGTCTTTGCTGCTCCCTCTGATCAC |
| | | | | | | 3573 | |
| | | | | | 21 | 32713 | CGCCAGAATTCAGACACACAGGGGCATTTTTGGTGGAAACCACAGAAGCT |
| | | | | | | 290 | |
| | | | | | 22 | 97646 | GTCAAATGTCTTCTCGCCCTGGGCTTTTTGTTCCCTAAAATGAAACTTCG |
| | | | | | | 341 | |
| | | | | | 23 | 10849 | CGGGCCTGTGCCTCAGACTCCGCCACCCAGCCACGTTAAGGTGACTTGGG |
| | | | | | | 0371 | |
| | | | | | 24 | 60091 | CGCACGTGGGTGCACATGTGTATGTGAGTTTGAGATAGAAAAATGGGAAG |
| | | | | | | 171 | |
| | | | | | 25 | 14298 | CGCATCAAGGTTCTCTGGCACTCAGCTCATCCAGCAGTTTGTCTCCTGCC |
| | | | | | | 4613 | |
| | | | | | 26 | 13959 | GAAGTGCCTGGAGGTCCACTGATTTTCCAGGCACCCTTTCCCGAGAAGCG |
| | | | | | | 2612 | |
| | | | | | 27 | 20178 | CGGCTTAGGTCCCACTGTACAGATGGGAAAACCGAGGCCTAGAGAGGGAG |
| | | | | | | 496 | |
| | | | | | 28 | 48982 | CGCTGCCCCGGCCCCACGGCCGGTCTCACTCGACTGTGAGTACACAGCGC |
| | | | | | | 683 | |
| | | | | | 29 | 99594 | CGCTCCACCAAGGGGCACTGCTGTCAGACAGCCAGGTAGCAAGTCTGTGG |
| | | | | | | 893 | |
| | | | | | 30 | 84671 | GCAAACCTTACAGGAGTGGTTCTTACTAAAATTCACACGCAAAGTACACG |
| | | | | | | 333 | |
| | | | | | 31 | 69136 | CCTTCTGGGACTCTCTGATGGGCCACACACCGAGCAGCTGCTATTTATCG |
| | | | | | | 44 | |
| | | | | | 32 | 15663 | CGGCCCCCAGACCCTGCCTGGGACGCCCCCCAACTAACTCCCTGCTGGGA |
| | | | | | | 74 | |
| | | | | | 33 | 44332 | TGGCAGGAAATCCCCTCGCCTGGGCGCAGACGTGGGGCGTGAACGGTGCG |
| | | | | | | 653 | |
| | | | | | 34 | 10276 | ATCACAAGAGGGTCGCTGCGTGGGAAAGATCCACCTCCCTCGGGGAACCG |
| | | | | | | 375 | |
| | | | | | 35 | 13933 | CGCCCTGTCCTGCACCCAAAGGAAAGGGCTCCCCTGGCACTCTCAGAAAA |
| | | | | | | 247 | |
| | | | | | 36 | 19278 | TAAGAACAGGATTCAGACTCAAGTCTTTTTGACCACAAAGTAGTACATCG |
| | | | | | | 249 | |
| | | | | | 37 | 45286 | CGGCGGGACAGGACCTTAACCCCTGGGACGAACACCAGCTCCGCAAAGGA |
| | | | | | | 062 | |
| | | | | | 38 | 72740 | GCATTTAAGAGAATGGCAAGAAGGCATAGGGCGATCCAGGCCTCGCTCCG |
| | | | | | | 460 | |
| | | | | | 39 | 70035 | CTGCAAACAGTAAGATTAGAGACCACTGGTAGCCAATCTAATAGTCCACG |
| | | | | | | 208 | |
| | | | | | 40 | 78511 | CGCCCGCCCGCCCAGCCTCCCCCGGGATGGATCTGGAGAGAAAGAAAGTT |
| | | | | | | 600 | |
| | | | | | 41 | 56228 | AAGCTCAAGAGAAGCCCCAAGCTAAATTACCCCTGGCTCAGAAGGGTGCG |
| | | | | | | 114 | |
| | | | | | 42 | 19691 | CGGTGTAGGCTAATTTTGGAGGGAGTTCCACATTATTATCAATTTTTTTT |
| | | | | | | 654 | |
| | | | | | 43 | 96970 | CGAGCAGCATTTTTGCTGGAATGCTTGGTGTAAGATCAGTGGCATTAAAA |
| | | | | | | 381 | |
| | | | | | 44 | 11381 | CGGCTAGATTGCTAGCCTTGGCTGCTCCATTGGCCTGCCTTGCCCCTTAC |
| | | | | | | 8635 | |
| | | | | | 45 | 37673 | CGCTCTTGTTCTGGCCCCCACTCCTGCAGGCCACGCTGCGGATGGTTCAG |
| | | | | | | 597 | |
| | | | | | 46 | 24722 | AGGGCGGTGAGATCTACAGGAGAACTCAGGGCGGATGTCTGCCGGGTGCG |
| | | | | | | 944 | |
| | | | | | 47 | 13181 | CAAATTTCTCAGTCCCCAAGCAGGCCACGGGGACTGCAGCAAACAGAGCG |
| | | | | | | 2449 | |
| | | | | | 48 | 10697 | CGAACTGCTCCATGGAGCGCGCGTCTCTGCAAGCCAGGAGAAAAGACCCT |
| | | | | | | 9451 | |
| | | | | | 49 | 10139 | CGACGCCCCTTGCACTGAGCCCTCCATCCATTTTAGTGCTGGAAATGGGG |
| | | | | | | 7146 | |
| | | | | | 50 | 60285 | GGGGGGCGGGGTTCTTCGTATTAACGATTGCCCGTGGTCCTCAGCTTGCG |
| | | | | | | 691 | |
| | | | | | 51 | 10089 | CGGACATTCTTTAAGAAGGTGCTTTGTATGTCTGAGACATCACAAGCATT |
| | | | | | | 3665 | |
| | | | | | 52 | 21984 | CGAGGTTGGCGACTGTTTTATCCAGAAGGAAACTATGCCCAACAGGAACG |
| | | | | | | 8333 | |
| | | | | | 53 | 22904 | AATCGACACAGGAAGCCAGAGGAGTGGCGCGCAACCAGTGGGGAGTGTCG |
| | | | | | | 6785 | |
| | | | | | 54 | 67070 | CGGAATGCTCAAGTCTGTCACCACACAGCTGTCAGCCAAGGGAGGTGCAG |
| | | | | | | 170 | |
| | | | | | 55 | 26240 | CGCCCAGTAAGTTACGGAAAAGGCGGAGACAGAGGTTTCGTTCCCGCCCC |
| | | | | | | 505 | |
| | | | | | 56 | 79502 | TTCAACCACACAGCTTTGATTTTGTCTTTCCATTTTCAGTAATGAAGTCG |
| | | | | | | 25 | |
| | | | | | 57 | 15814 | GATCTAAAAGGATTGGTGTGGAGGCCAGAGGGCTCCGGTCCCCGCGCTCG |
| | | | | | | 4020 | |
| | | | | | 58 | 45406 | CGCTGCTATAAAAGGGGTCCCGCGCGACTTCCAAACTCAGCGCCAACCCG |
| | | | | | | 486 | |
| | | | | | 59 | 65731 | CGCCGGGGCCTCCTTAAAACACAGCGCACTCGAAGCGCAGACTGCTGCGC |
| | | | | | | 689 | |
| | | | | | 60 | 86383 | CGCAGCTCCTCACCCCGCCTGCTCCGCAGCGGGACCGCGTAGACCTCAGC |
| | | | | | | 430 | |
| | | | | | 61 | 26552 | TTGCGTTGCCCACAAGCTTGTAGTCAGAAGAACTCGCCTGGGAGGGATCG |
| | | | | | | 116 | |
| | | | | | 62 | 27528 | CGCTTAAGCTGGTAGACCCTATGAGTGTGTGGCATTAGGCATCCCCAGCC |
| | | | | | | 611 | |
| | | | | | 63 | 11178 | CGGGCGGCGCTGGTCACACCCTCGTTGCTCTCACTCAGGGCCCCCACTCC |
| | | | | | | 3348 | |
| | | | | | 64 | 24254 | CTTGAGTGAGCCCTGTGACGCCTGCTAGGTGAGGTGGCAGCCAGTGAGCG |
| | | | | | | 9864 | |
| | | | | | 65 | 22959 | ATGCCTGGCCCAAAGCAGGGGTGCAGGCAGCTTAGGGATGACGAGTGTCG |
| | | | | | | 868 | |
| | | | | | 66 | 79278 | GTCGAGGTGTCTCGCGGCTCCTAATGTGGCTGTCTGGCTGCGTCGGGACG |
| | | | | | | 012 | |
| | | | | | 67 | 76209 | TAGGCTCCTTTGTGGAGACCTGATTTGAGCACTGGTGTGAAAAAGACCCG |
| | | | | | | 541 | |
| | | | | | 68 | 15852 | CGCCCCTTCAGGCGACATAGACCCAGCTGACAACGGGACACACATGTCCC |
| | | | | | | 4270 | |
| | | | | | 69 | 40677 | CTTTCAGAATTCTCTCCTGGTGGCTCCCTCTTTCCCTTGGGAAATCTTCG |
| | | | | | | 600 | |
| | | | | | 70 | 70378 | CGTCCTCAAGCACTCAGCACAGTGGTGTGTCAAGACGGGCTGCCCAATCC |
| | | | | | | 994 | |
| | | | | | 71 | 10545 | GGGAGGACCCGGACGGGAGCAGGTTCCCTTCGCGCCTCTCTGGAGGGTCG |
| | | | | | | 9775 | |
| | | | | | 72 | 31020 | GGGAGTCGGGGGCTAAGGGCTCGAGCCAGGAGGCCCCGGAGCAGCAGCC |
| | | | | | | 754 | G |
| | | | | | 73 | 16470 | CGCACGGAGCTTGCTCTGGCCATCTCTGTCCCTGCCTGTGCCATGGACAT |
| | | | | | | 16 | |
| | | | | | 74 | 21666 | TCCACGAGGCGTTCGGACACTTTTTCCCGGCCTGACAGCAGAGAAAGTCG |
| | | | | | | 778 | |
| | | | | | 75 | 29034 | CAGGGCGGTCGAAAGAGCCTTGGGGGCACATCTCGGGGTGCGGTGACCCG |
| | | | | | | 163 | |
| | | | | | 76 | 13702 | GAGTATTCTGCTTTTAATAAGCTTCCCAATCAGCTCTCGAGTGCAAAGCG |
| | | | | | | 8535 | |
| | | | | | 77 | 18891 | CGGGGCCCCCAGGCACCCGGCCCGCACGCAAACCCTCAGCCCGGGGCCCC |
| | | | | | | 6709 | |
| | | | | | 78 | 11954 | TACAATGGCAAAATGCACTGTTGAGAACCCTTTGAAGTTGGGCAACCACG |
| | | | | | | 2475 | |
| | | | | | 79 | 45914 | GCTGGGGCGAAGATGAGTCCAGGGGTTGGGTTGAGAGCGCTGCGCAGCCG |
| | | | | | | 840 | |
| | | | | | 80 | 53030 | GGGCCTGGGTGGAAACCAGGAGGACGGATTGCCGCCTGGGGCGCTCGCCG |
| | | | | | | 043 | |
| | | | | | 81 | 13953 | GGCTTTCTGCCAGCTGGGGACACAAGATATAACTTTCATGCCCAGCTCCG |
| | | | | | | 7131 | |
| | | | | | 82 | 74025 | CGATGCCCACCAGCCTTACCGATTTAGGTAAAGGAGTAAATAAATTGGCA |
| | | | | | | 019 | |
| | | | | | 83 | 14507 | CTTGAGGATGCCTGAGGTCCACGTGACCAACTAGGATCTTCTGGCATTCG |
| | | | | | | 7291 | |
| | | | | | 84 | 10289 | GGCTTCTTCTTCTTGGGGGGCGTCCGGTTCTGATAGGGGTGACCTATACG |
| | | | | | | 3925 | |
| | | | | | 85 | 53092 | GCCACTTTCGGGGAGCGCAGAGCGCGGCGCGGGGAGGCGAGGCTGGAGCG |
| | | | | | | 233 | |
| | | | | | 86 | 33701 | CGCAGCTGCAGGGGCGCAGAGCACCCTCTATCCAAGCTGACAACCATCAG |
| | | | | | | 209 | |
| | | | | | 87 | 54357 | CGGGATGTAAAAGTGTGGAAAACTTATTTCCCACGCAGAACCCTTGGTCC |
| | | | | | | 994 | |
| | | | | | 88 | 74404 | CGAGTTGATATTTAATTAGTGAATGCACAGTTGCTTTGTGGACCTTAATA |
| | | | | | | 556 | |
| | | | | | 89 | 38066 | CGTGGGAAGAAGCACCACATTCCTGTGGTCTTGGTTGACTGCTGAAATGC |
| | | | | | | 983 | |
| | | | | | 90 | 85674 | GGCGGTAGGAACTGAAATCGATCACAGCTCGTTAGAGTACGTCACTGTCG |
| | | | | | | 694 | |
| | | | | | 91 | 52536 | GGGGTAAAAGCTCCTGGCCAGGGCTGCCTGGAGCTGCCCCTTCCACTCCG |
| | | | | | | 022 | |
| | | | | | 92 | 88583 | AACCAAGGAGACCAAAACCAGAATTCTCAAATCTAAAGTGGTTTAGGCCG |
| | | | | | | 117 | |
| | | | | | 93 | 89744 | CGGCAAATTGGGGGTGACGTTCAAGATGTACTTGATGCCGAATTCCTCCA |
| | | | | | | 471 | |
| | | | | | 94 | 42007 | CGCGTCAACTGAAATATTCAGGTTCTCACATTGGGACTGACTAGGCAAAC |
| | | | | | | 378 | |
| | | | | | 95 | 40588 | CGCCTTACCCTGTTAACCAGAACAGCCATTGCATATTCCGTTCTGGGTTA |
| | | | | | | 553 | |
| | | | | | 96 | 22954 | GTGGCAGATAAGACACGAGAGGGAGCTCTGGTCCCCGAGGGGCTGTCCCG |
| | | | | | | 3254 | |
| | | | | | 97 | 15627 | CGGCTACAGAAGCAGATTTTGAGGGTGGAATTAGAACACTTAAACACAGG |
| | | | | | | 1246 | |
| | | | | | 98 | 49309 | CGCAGCGTTTCCGGCACCCAGCCAGGCTTAAAGGTACTGTGAGGGGAACT |
| | | | | | | 445 | |
| | | | | | 99 | 15727 | CGCCCGCCAAGCAAGTAGCAGCTTCAAAGCCCTGCCCTCGCTCTCGCATG |
| | | | | | | 441 | |
| | | | | | 100 | 12788 | CCCTGAGGGGCGGGGCGGGAGCTGGCGCTAGAAATGCGCCGGGGCCTGCG |
| | | | | | | 1269 | |
| | | | | | 101 | 46236 | CGGCCTCTATGGAAGTCTGCACTCTGCTTGCTGTGGTCAGACTCCAGGGA |
| | | | | | | 351 | |
| | | | | | 102 | 10031 | GGATTTAGCGCCCCGGCTATTGGCCAGGAGGTGGCTGGGTTCAAGGACCG |
| | | | | | | 8718 | |
| | | | | | 103 | 87230 | GAGGTGCAACGGAAGCCAGAACATTCCTCCTGGAAATTCAACCTGTTTCG |
| | | | | | | 344 | |
| | | | | | 104 | 56985 | TGCCAGTGTCCAATAAAACCAGTCAGGTCTCACATATTATGTAGTTAACG |
| | | | | | | 462 | |
| | | | | | 105 | 47695 | CGAACCCTCCAACTGGGATCGGTCTGGTTCAGTGTTTGTTTTCTAAGCAG |
| | | | | | | 321 | |
| | | | | | 106 | 39694 | GGGTAGGTGCCGGCAGAGAACTTGTGCGTCTTGCCCGCCTTGCCTTGCCG |
| | | | | | | 617 | |
| | | | | | 107 | 57976 | CTCAGGTGTCTCAGGCCCACGGAGGATTTAATAGATGAAACCCTGGATCG |
| | | | | | | 915 | |
| | | | | | 108 | 17702 | CGCTTCGGTAAGGAATGAAAAGAAAAACACTTCCCAAGGCCCGGCACTTT |
| | | | | | | 9921 | |
| | | | | | 109 | 38266 | CGCAGGCAGCCGTGGACACGATCGGGTTTCCTGTGCCAGTCAATTACATG |
| | | | | | | 21 | |
| | | | | | 110 | 17513 | CGGTCAAGACACAGGAACAGCCGTACTTTCCTGCGGGTAAGAGGCTTTTC |
| | | | | | | 2871 | |
| | | | | | 111 | 27153 | CGCACCCCCGGACCAGACGCTGCTCCGCGTGTAATATTCATAAGAAACAT |
| | | | | | | 663 | |
| | | | | | 112 | 22316 | GCACGTAATTTCGCAGTCTTCTGTCCTATCCTCATGTTACAGCACCGACG |
| | | | | | | 2049 | |
| | | | | | 113 | 36994 | CGGGTCAAGCCCCACTAGAAGTTAACACCTGTGGTCTCCAAACTTGCCTA |
| | | | | | | 016 | |
| | | | | | 114 | 58357 | CGTCCAATAAGAAGAGCCCCAGCTTGACACCTGCCTATATACAGGCGAGC |
| | | | | | | 973 | |
| | | | | | 115 | 13587 | CCAGGAGAGCAGCAGCGGGAGCGCAAGGCTCCAGGGCAGAGGCATTCTCG |
| | | | | | | 976 | |
| | | | | | 116 | 36044 | CGGGCCGGTTCGAACCGCTCCCTTAACCCTTAAGCAACCAGGACGCTCCC |
| | | | | | | 860 | |
| | | | | | 117 | 36919 | CGGGAGGGCAAAAGACCACAGAGATCTGCGAAGCCAAAGTAAACAACGG |
| | | | | | | 960 | G |
| | | | | | 118 | 20716 | CCAGGGGCGCAGCGGTGTGCGCATAGTAGGGTGGGAACCCGATGGCAACG |
| | | | | | | 638 | |
| | | | | | 119 | 40573 | CGCCCCCACCCCCCGGCTCCGCCCGGGGCCACTTGTTGCTTTCTTTGCAG |
| | | | | | | 764 | |
| | | | | | 120 | 50373 | CTGAAGCATCACTCACAACTCCACTTTCTTTACTTAAATAAGGATTTCCG |
| | | | | | | 735 | |
| | | | | | 121 | 40138 | CGCAATCCCCAATCCGCTGCAGCCCACAAACCCAGAGAGCCAGAGAGCCC |
| | | | | | | 052 | |
| | | | | | 122 | 28388 | CACACGGGGACGGAGCTGGGCTGGCAGCGGTGCCCAGGACAGAGTGGGCG |
| | | | | | | 05 | |
| | | | | | 123 | 11067 | CGCGCACGATAGTCCCAACGCGGGCCTGTCTCACCTGGACTTCCTTCCCG |
| | | | | | | 3082 | |
| | | | | | 124 | 53286 | CTGTGAATTCCACCCACCGGGCATGGGAAAGTGCGGCATTCCCAGTCCCG |
| | | | | | | 921 | |
| | | | | | 125 | 41011 | CTGTTGGCTTCTTATCACTGACAGCGGAGAAAGACTGATGGGCCTTGGCG |
| | | | | | | 32 | |
| | | | | | 126 | 49519 | CGAGCTACTCATTAGAGTTTAATTTCCTCGAGCCAAGTCCCTGGAGACGG |
| | | | | | | 250 | |
| | | | | | 127 | 14511 | CTGGAGTCCCACAGGAGCTCTTCAGCTGGTAGCCCTGGAAAAACTGGACG |
| | | | | | | 4994 | |
| | | | | | 128 | 15759 | CGGCTACCTGGAGGGCCTCCTGCCACCGGCCCGCAGCCTGGTAGAACTTG |
| | | | | | | 79 | |
| | | | | | 129 | 13162 | GAGGCTGCACTCACGAGGTCCGAGTGGAAGGGCTTCGCGTTGGAGGTGCG |
| | | | | | | 2739 | |
| | | | | | 130 | 57504 | CGGGCACTTCCAGGCCCCATAGGTCTGTAGCTCTGTCCAGCGAGTTCAAG |
| | | | | | | 948 | |
| | | | | | 131 | 23360 | GAGGATGACCCTGTATTCCTGGGTTAGTCAAGGTCAGCTTGCCAGAGACG |
| | | | | | | 34 | |
| | | | | | 132 | 76137 | GTAGAGAGTAACCAGCACCAGCTTCACAGGAATGGCATGACCTTGATACG |
| | | | | | | 0 | |
| | | | | | 133 | 22094 | TTAAACTTCATTAGACACTGAAACTTGGAATATGATTCCATGATTCTCCG |
| | | | | | | 461 | |
| | | | | | 134 | 17073 | CTATTTCACTTAACGCCGCTTTTATTTCGTTTTCGTTCCAATAATGCTCG |
| | | | | | | 9179 | |
| | | | | | 135 | 77591 | GTAATTTAATTTTCTCTCCCGAAAGCGTTGCTAATTAAATCCTTTCTCCG |
| | | | | | | 726 | |
| | | | | | 136 | 50690 | CGCCTCCATGCAGCCACGACCACCGCCCTGAGCCGGCCGCAGCGCCACCT |
| | | | | | | 152 | |
| | | | | | 137 | 19558 | CGCGGCTTGGGTTTCAACGCACTTTGGCGCCTGACAGAGTCGCATCTCCT |
| | | | | | | 808 | |
| | | | | | 138 | 45949 | CGCCCGGCCTGTAGCTCCCCCCACGGCCCAGACGCAGGGAACTCCCCTCC |
| | | | | | | 828 | |
| | | | | | 139 | 16097 | CGCTGACTCAGGAGACTTCGGAGTGTAGGCTGTCTAGTTCAGTGGTTAGG |
| | | | | | | 5792 | |
| | | | | | 140 | 13928 | AGGCCAGACCTGCAGGCATGAGAGGCACAGGCAACCCGCCTGTACGGCCG |
| | | | | | | 4498 | |
| | | | | | 141 | 10289 | AGGCCTCCTGCTGCAACTGCAGGAGGATGCGGTTCGCTTGCTGCCTCTCG |
| | | | | | | 6475 | |
| | | | | | 142 | 89954 | GCACTGCACCAATTCACACATGGATACAGACTTCCAACAAATGACTATCG |
| | | | | | | 679 | |
| | | | | | 143 | 21022 | CCTGTCCACCTGGAAGGCTGCCCGCTCTCCCTGAGGGTCCTGGGAAGGCG |
| | | | | | | 844 | |
| | | | | | 144 | 61733 | CGTCACAGCTACCTGTCTCTGGGGATCCCTAGTATAACACATTCAGTGTT |
| | | | | | | 046 | |
| | | | | | 145 | 25090 | AAGTGGGGGGCAGGGGCTGTGCGCGGCTCTCTCCCCTAAAGCGAGGTTCG |
| | | | | | | 298 | |
| | | | | | 146 | 55488 | TTGGGGGCTGGGTCACGGGATGAACCGGGGGGTGGGTCACGGGATGAACG |
| | | | | | | 6 | |
| | | | | | 147 | 13205 | CCAGGCGACTGGGAGAAATTTGGAGACCTGTCAGAGTGGTCAGACACCCG |
| | | | | | | 3823 | |
| | | | | | 148 | 10091 | CGGGTAAATCGGCAGCGAGACAGCCCCCTGCAAATAATGCTGGCATTCCC |
| | | | | | | 1949 | |
| | | | | | 149 | 26172 | TGGGCGTAACGGGGCGGCGGCGTAACGTGTGGCTCTGTGGTTAGGGCACG |
| | | | | | | 185 | |
| | | | | | 150 | 15094 | CGCCCCAGTGAGGGTGATCGTATTCTCCTAAGGAGAGCTTTATTGCTGGG |
| | | | | | | 9495 | |
| | | | | | 151 | 36132 | CGGCCAGATTCCCCCACCCGCCCCCAGCGTCAGCTGACGTCACAATCGGC |
| | | | | | | 627 | |
| | | | | | 152 | 13622 | CTGATGGGGGTCCACTGGGACTTACATTTACACAGAGCTCGCTGGTACCG |
| | | | | | | 4666 | |
| | | | | | 153 | 33700 | CGGCAGAGTTCGCGGGCTTAGCAGACCCGCCCATTGGCTCTTGCATCTCC |
| | | | | | | 757 | |
| | | | | | 154 | 62304 | TCTTTATAAAGGGCCGCCTTGGGGCGCGAAAAGTTCGTGAGGTGTAGTCG |
| | | | | | | 554 | |
| | | | | | 155 | 10003 | CGCAAGGGGGCTGAAGAGCCAAATGGGGAAACTGATGGAGCCTCACAAGT |
| | | | | | | 9476 | |
| | | | | | 156 | 16206 | CGCCTGTCGACGAGATGCTATTAACCAGTTTTTGCTCTGCCCCTGTCATG |
| | | | | | | 8382 | |
| | | | | | 157 | 36919 | TTTTACTCTTTACAATACATTCTTTCATGCTTCCTAGGGATTGAGCTGCG |
| | | | | | | 819 | |
| | | | | | 158 | 97176 | CGTGCAGACAACAGAATGGGGATGGAATCTCCTTGTGGCCCCCAGCTCAG |
| | | | | | | 7 | |
| | | | | | 159 | 23165 | CGCTGGCGTGCAACACAGGCACTGACTCACACTTTGTCTAAACAAGCTTG |
| | | | | | | 8979 | |
| | | | | | 160 | 16228 | TGCGGTCGGTGGGGCGCAGGGCCGGGGAGCCAGGGGGGTGCCGGGGCTCG |
| | | | | | | 3705 | |
| | | | | | 161 | 12645 | CGTGGTTGCTCGACTGTTGTATCTGTGATACATTATCCGACTAAGGACTC |
| | | | | | | 0465 | |
| | | | | | 162 | 10005 | GAGCTTCTTTGAAGACCCTCCTCAGATAAACTGCTGAGTTGCAGGTGTCG |
| | | | | | | 3305 | |
| | | | | | 163 | 11954 | CGTGGCGGTCCCCTTGCTCATCTGACTCCGGAGGCAGGTTCCGCAGAAAG |
| | | | | | | 3336 | |
| | | | | | 164 | 40120 | GGACACAGTGGCCACGCTGCTAGAGTGCAAGACGCTCTTCATCTTGCGCG |
| | | | | | | 250 | |
| | | | | | 165 | 57347 | CGCCCTTGTTGACTTTCAAAAGGCGCCGCCGGCCCCGCGCCCTACACGCT |
| | | | | | | 16 | |
| | | | | | 166 | 21489 | TCCACGGGCTCCATGCTGGATTCCATATGGCCAGCTGGGCCGAGGGAGCG |
| | | | | | | 196 | |
| | | | | | 167 | 39755 | CGCCCAGCCCTTGCGCCCCCCGGTACACTGGCCCCGCCGGGTTCCCACAC |
| | | | | | | 181 | |
| | | | | | 168 | 73847 | CGCGCCCTTCTGCACGGGGACCCCAGCCCACCCCCGTACGGACACGCCTT |
| | | | | | | 259 | |
| | | | | | 169 | 74035 | CGCAGGCCAGCAAGCTTCTGAAAAGCAAACCTAGGAAGTAGCTTTCCAAC |
| | | | | | | 742 | |
| | | | | | 170 | 29632 | TCGTCCAGTTGACTCCAAGCTTGATAACGCGTGTAGGGGCCGTGAGGCCG |
| | | | | | | 64 | |
| | | | | | 171 | 25129 | CGCCCCCAGGGCCCGGCGTCCTGGGCCTCTGACTCTGCCCGGTTTGCTAA |
| | | | | | | 184 | |
| | | | | | 172 | 32595 | CGAGAGAATGAGTAGCCTGAGCAAAGTCATAGGGGTAAGATAAAACGACA |
| | | | | | | 801 | |
| | | | | | 173 | 64937 | ACAGAAAAACTGCCCGCAGCTAGTAAGGAATCCTGATGATGTTACTCACG |
| | | | | | | 528 | |
| | | | | | 174 | 95235 | CGTGGGCACGCTGTCGCGCACCGAGCTGCAGCTTCTCAACCAGCTGCACT |
| | | | | | | 489 | |
| | | | | | 175 | 38892 | CGGTGGCTATCAGACGAAGAATGGGAAAATACAGATAGCAGACTTACAGT |
| | | | | | | 901 | |
| | | | | | 176 | 10333 | AATGCGCCCTGACTCCCCCTGGCCGAGAGCCGGCTCAGAATGAAAGAGCG |
| | | | | | | 766 | |
| | | | | | 177 | 26271 | TGGAAAATCTGATTGGATGAATCACTTCATTTTTGAATCCTCTCTTCTCG |
| | | | | | | 816 | |
| | | | | | 178 | 11410 | CGGGATAAGGTTAGCTCGTTTATCCAACAAATATTTGCAGAGCCCCTACT |
| | | | | | | 9526 | |
| | | | | | 179 | 31820 | CGGACTAAGTGGGCCAGACTCTTGTCAGGGGAGGGATCACCAGGACCTCA |
| | | | | | | 572 | |
| | | | | | 180 | 74810 | AGGCTACCAGGAGCAGGAGCAGCAGCGCCGCCCGCAGTAGCCGGGGAGC |
| | | | | | | 017 | G |
| | | | | | 181 | 50227 | TAGATGAGGCCCGGCGGGTTCCTCCCCTCAGGCTAGGCGGGGTGGCAGCG |
| | | | | | | 199 | |
| | | | | | 182 | 10184 | TCTGAGGGAATACCCGCAGCAGCGCTACCAGAGCGCATTTGCTCTATGCG |
| | | | | | | 1008 | |
| | | | | | 183 | 73101 | AACTTACAGTCTTCAGGGAAGGGCTTCCTGCCAACTGGAACAGTGGTGCG |
| | | | | | | 264 | |
| | | | | | 184 | 67990 | CGGCCTCCACCCGCCACAAGCGCACAGGGCTCTGCCCACTCCCCGCCCCT |
| | | | | | | 0 | |
| | | | | | 185 | 70682 | ACCTGGGCGTGGGGACACCACCAGGCAGGAGCAGAGGCAGGACTGGGAC |
| | | | | | | 098 | G |
| | | | | | 186 | 53097 | AGGGCGCCGGCAGGCGGCTGAGGCAGAACGGTTGGGCTGCGGTGCCTCCG |
| | | | | | | 777 | |
| | | | | | 187 | 19754 | CGGAGCCGCTGCACCACCACCCCTACAAATATCCGGCCGCCGCCTACGAC |
| | | | | | | 125 | |
| | | | | | 188 | 12934 | CGGAAGTGACCAAGCGCGACTAGATCTAAAGCCTTCCCGACTTCCATTTT |
| | | | | | | 6694 | |
| | | | | | 189 | 62070 | CGGACACCCAGAGCCCAGCGCGCCCGCCCTGCGTCTCCGCACCAAGCCCT |
| | | | | | | 184 | |
| | | | | | 190 | 28603 | CGCGGACTCCTTGTCCTTGCCGGTTCACCTCCCTGTGGAGGACCTCCTCT |
| | | | | | | 292 | |
| | | | | | 191 | 10139 | CGAGCCAGGTCAGTGGGCACTGTTGCTAGGTCCTCTATCATGTTCCTGGG |
| | | | | | | 5217 | |
| | | | | | 192 | 32223 | TGAAATCTTGCGGTGTTGAGTAGGACCGCCAGGCCGGCAAGCAGGAGGCG |
| | | | | | | 076 | |
| | | | | | 193 | 13702 | CGAGAGCTGATTGGGAAGCTTATTAAAAGCAGAATACTCCAGGGGCAGTC |
| | | | | | | 8546 | |
| | | | | | 194 | 36728 | ATTAGGTGCCTTCGGCGTGGGCCGGCCCCCACTCCAAAATCAGGCTGGCG |
| | | | | | | 693 | |
| | | | | | 195 | 70151 | CGCCAGGCTTGCTAGTGAGGATGGGACGCAGTCTCAGGCCCCACGCAATC |
| | | | | | | 160 | |
| | | | | | 196 | 96463 | CGCCGCGCGCGCTCTCTGCCCATCTCCGGCTTCGCTGAAGCTGCTACAGC |
| | | | | | | 902 | |
| | | | | | 197 | 96626 | CGGTCATCAACTGAAATAGGCAAAACCAATAACGCACCTCGCTTTTCGCC |
| | | | | | | 788 | |
| | | | | | 198 | 10675 | TAAGGTGTCTTCCTACAGACACCGCTGCTGCTACCTTGCTACCTTCAGCG |
| | | | | | | 0146 | |
| | | | | | 199 | 13169 | GGCGTCTTCTGCGCATTTGTTGAAGTTACAGAGCCCTCGTCATGATCCCG |
| | | | | | | 4034 | |
| | | | | | 200 | 62583 | TGGGAGGCTGGGGCTGGGCGAGACTCGCGGGCCCGGGTAGGTGAGCCTCG |
| | | | | | | 795 | |
| | | | | | 201 | 18254 | CGTGGTTCCAGGCTCTTGGCTGGACCGGGAAGACCATATGGCGCATGCCG |
| | | | | | | 5485 | |
| | | | | | 202 | 89954 | CGGTGCTGTGGCATAATTACCGTGCTCGTTGACTTATAGTCTCATGATTT |
| | | | | | | 258 | |
| | | | | | 203 | 63026 | CGCTCTTTGTAAATACAGAATAGGACAAAACGGAAAGAGTGATGTGCTGT |
| | | | | | | 212 | |
| | | | | | 204 | 13356 | CGCCGCACAGCAACCCGAACTTTCGTTTGGTAGCACTTGAAAGAGTTTTC |
| | | | | | | 3764 | |
| | | | | | 205 | 27637 | AACCACAAACCTGGTAGTCCAAATGTGTTGGGCTGTGGCCAAGTTGAACG |
| | | | | | | 395 | |
| | | | | | 206 | 29974 | CGCGCCCGCGTCTTCATCCTCAGACTCACGGCGTCACTGTCGACCCGCAC |
| | | | | | | 723 | |
| | | | | | 207 | 21022 | CGCAGAGCCCCTGTTGCCCTGGGCCAGATGGCCCGTAGCAGCAAAAATAA |
| | | | | | | 699 | |
| | | | | | 208 | 34530 | CGCCCCACAAGCAAAAGTAATTCCCAACCAAGCATGCGCAGTGCTCAGGG |
| | | | | | | 799 | |
| | | | | | 209 | 79234 | CGCAGTATGACTTCAAGAAGCAGCCTCATGGTTAAGAGAAAGGGGTTCTG |
| | | | | | | 251 | |
| | | | | | 210 | 62304 | CGCGGCCGTCAGGCAGTCAGGGCACCCACCCACTGTCCGACTCCACTCAC |
| | | | | | | 779 | |
| | | | | | 211 | 48050 | CGCGTGGAGCGTTTCAACCCCCACGTTCACCGCGCCAGGTGATACAAGAC |
| | | | | | | 274 | |
| | | | | | 212 | 96463 | CTGCCCCTTGGAGCTGTTGCCTGGGCAGGGGTGGCCTTGGTTGTTTACCG |
| | | | | | | 185 | |
| | | | | | 213 | 22316 | AAGGTCGAAGAGTCGTTACCAAAACCTTTGAAGCCGCTTTTCGGCTCTCG |
| | | | | | | 9965 | |
| | | | | | 214 | 10270 | TCGTGGAGCACGGCGCTTGTGTAGGGCAGAGCCTGCTGGTCCTCCAGCCG |
| | | | | | | 33 | |
| | | | | | 215 | 93074 | CGCCCAGCACCCTGCCTCCGGCGATGCCGCCCCAGCCCCTCTGATGGCCC |
| | | | | | | 929 | |
| | | | | | 216 | 19692 | GAGCGCATACCAAAGGCAGACTCCTTTGCAGGCTCCACGAGCCCCATTCG |
| | | | | | | 217 | |
| | | | | | 217 | 17702 | CGGTGGTCAGCCAGCTCCCTGCATTGTTCCGGTAGCGAGCTCCACACACA |
| | | | | | | 6301 | |
| | | | | | 218 | 54430 | CGCCCCACAGCTGGCTCCCGCGCTCTGCCCACCGCCACGCCACCTTCACC |
| | | | | | | 382 | |
| | | | | | 219 | 76101 | CGCTGACCCACCGCAGCCCGCACTGTTTTGCGAACAGTTCGCAAGGACTG |
| | | | | | | 114 | |
| | | | | | 220 | 31938 | CTGCCTCTCAGGGGTCCAGGCTGGCTGGCAGAGGCCATAGTGACGTGGCG |
| | | | | | | 886 | |
| | | | | | 221 | 97000 | CGGTCCAGGTGGGGAAGAACTGATGCAGAAATGCTATGTATACATTAAAT |
| | | | | | | 811 | (This probe can belong to Red or Green) |
| | | | | | 222 | 10090 | CGGCCACTGTCCCTGAAGGGCAAGCAGTCGTCTAAACCTTAGGTCCAGAT |
| | | | | | | 6383 | |
| | | | | | 223 | 12568 | CGCGTGTCTGATACTTGGGATCCTGGCTTGCGGGCCCTCCTAGACTCTGC |
| | | | | | | 7041 | |
| | | | | | 224 | 12920 | CGCCCTGTCCCCTGGCCTTGGCTTGTGAGCTGAGCGAGCAGCAGCTGCTG |
| | | | | | | 4339 | |
| | | | | | 225 | 22959 | CCGAGCTGGTGGTAAATTGCATCTCTCGACAGAGGTGGCTGAGGGAAGCG |
| | | | | | | 525 | |
| | | | | | 226 | 10485 | CGGAACGTTCTACCTGTTGGGCTTCGCAGCCGAGCTGCTCACCCCCGGCC |
| | | | | | | 2355 | |
| | | | | | 227 | 14604 | CATTGCATCAAGCCTTGAGTGTGCATGATAAGCAGGATGTATGCCCTTCG |
| | | | | | | 4461 | |
| | | | | | 228 | 12265 | TCTATATATACTCAGGCCTCCAGTTGGGGGAAGCCCTTGGTGGCCAATCG |
| | | | | | | 1486 | |
| | | | | | 229 | 37389 | CAAGATTCTGGGGACTCAGTCACTCCAGGCTGTGGGGTCTGGAGTGGTCG |
| | | | | | | 951 | |
| | | | | | 230 | 18546 | CGGCAAAGGCGCTGCTGCCTCAAGCTTGCTTTACATGCCTCTCTAGTTCC |
| | | | | | | 2493 | |
| | | | | | 231 | 14391 | CGGAGCCCCGCAGTGTGCGTGCCCGCCGCCTCCAGCCATCTGAGCCTCCC |
| | | | | | | 6273 | |
| | | | | | 232 | 10089 | AATGTCCTTGAACGTCATTAGCAGGTTTCAGGGACCTACTTCGCTATTCG |
| | | | | | | 6803 | |
| | | | | | 233 | 60032 | CGGACAGTTGCCCACTCACAGCGTGCAAAGGGGTCGCACTCCCTGTGGGT |
| | | | | | | 607 | |
| | | | | | 234 | 17275 | TCTCCGACGACAGCTAGTTTCTATAGTAATGTGGGGTGCGGGGATTCCCG |
| | | | | | | 1331 | |
| | | | | | 235 | 10128 | TGGAAAGCCCTGTGACCTATTCCCAGGGACCCAGCAGTGGGCTCCCAGCG |
| | | | | | | 2726 | |
| | | | | | 236 | 13959 | CCCACAGAGTATTCAGGTGTTAATCACCAGCTCTCTTTTCCCTATGTGCG |
| | | | | | | 2127 | |
| | | | | | 237 | 10374 | CTGCTGGAGGGCCCAGCCCCTGGGGCCTCGGGGTCGTTCGGGTCCTGACG |
| | | | | | | 0476 | |
| | | | | | 238 | 15162 | GAGCAGGGCAAGTGACCCATGTGTTTGCCTCTTCTACCTAGGAGATGACG |
| | | | | | | 623 | |
| | | | | | 239 | 13922 | GTGCCGCAGTAACGGCTGCTGCTCGGCCAGGCGGTAACTGATGGGCGCCG |
| | | | | | | 8153 | |
| | | | | | 240 | 10632 | TGGAAGCGAGTCCCATACCAGGGCTCACACCCTGGAAGGGGCTGGTTCCG |
| | | | | | | 861 | |
| | | | | | 241 | 11708 | CGCCCTCAAGCCGACTCCCGGGCCGGCCCCTCGCTGCCCTCCCGCAACCT |
| | | | | | | 6479 | |
| | | | | | 242 | 74734 | CCTCGCCCTTCAGAGTAACTCCTGTGGACTCTGAGACTCTGGGATATTCG |
| | | | | | | 874 | |
| | | | | | 243 | 70151 | CGCAATCCTTTTGCAAAGCCAAGAAGTGTGTGTGTTGTGTGTGTGTGTGT |
| | | | | | | 117 | |
| | | | | | 244 | 14937 | GGAAGAAGATCCTGCCGGAGTCTTTCTTTAAGGAGCCTGATTCGGGCTCG |
| | | | | | | 4914 | |
| | | | | | 245 | 20687 | CGCGCCAGTCAACTCCGCGCACGTTGCCCCTGCTTGGCAGCCAATAAAAG |
| | | | | | | 681 | |
| | | | | | 246 | 12285 | CGAGCAGGCTTGGAGACTCTGCCTGCGTCCTCCACTTCGTCCCGGCCCTT |
| | | | | | | 2454 | |
| | | | | | 247 | 59529 | CGCGGTGGAGGAGCCGGCGGCTCGCGTGGGGCTCGGGATCTTGGGGACCG |
| | | | | | | 618 | |
| | | | | | 248 | 57276 | ATCCATCCAAACGACCCCAACCAAATCTGGGGGCCAAAACGCAAAGATCG |
| | | | | | | 527 | |
| | | | | | 249 | 22317 | CGCCCGCAAAGACGCCGCATTCCAAATGCAGAGCAGATCGGCTGTAACAG |
| | | | | | | 6167 | |
| | | | | | 250 | 83636 | CGCCCGCCGGTAGGAAGGAAAGAAAGCAAAGCACATGCAAATTGACCGAC |
| | | | | | | 325 | |
| | | | | | 251 | 22316 | GACGGCACGGTGTTTCGATCACAGACCGCGTCCTTGAGTAATTTGTCTCG |
| | | | | | | 0297 | |
| | | | | | 252 | 10844 | CTATTATCACTCACTACATTTCCCAGCTATGTACTGAAAAAATCAGCTCG |
| | | | | | | 0118 | |
| | | | | | 253 | 56424 | CGGAGGCTGTACAGTCATGCTAATGAGCTGCTGGACAATGGCAGTTTTTG |
| | | | | | | 872 | |
| | | | | | 254 | 20145 | CGGAGAAAGCCTTGATTAAGATCCAACGTTCCTGAGCTCTTTGCCAGGAA |
| | | | | | | 0690 | |
| | | | | | 255 | 12644 | CGCCTGGGGTGGAGGGTATGAGGCAAAGCCCCAAATAGTATTCATAATGC |
| | | | | | | 8033 | |
| | | | | | 256 | 98196 | GAGGACTGTCCATGAGCGTACGCAGCTGCTCCGGGTTTTCGTGGCAGCCG |
| | | | | | | 97 | |
| | | | | | 257 | 10635 | CGGCACTCTCGATGGAGTTCCATTTTGTTGCAGTGGTGCTTCAGCTTAGA |
| | | | | | | 9455 | |
| | | | | | 258 | 19183 | CGGACCACAGCTCCTTGTCCCAGGGCCACAGTGGTGTTCATTCAGTGCCT |
| | | | | | | 732 | |
| | | | | | 259 | 15779 | CGGCCTCATCTTCGCCCAGCCAACCCCGCCTGGAGCCCTATGGCCAACTG |
| | | | | | | 999 | |
| | | | | | 260 | 72897 | AGGATCTGGAGCCAAGTAATGGTGATACTGATGCTTCCTTTTCTTTGCCG |
| | | | | | | 565 | |
| | | | | | 261 | 26273 | GGAGCCCTTCTTCGGGGCGGGAGCTGACTTAGCTGGTTCAGGCATGTTCG |
| | | | | | | 199 | |
| | | | | | 262 | 39523 | GAGGGGCGTGGGGAGCTTGCGGCACAGCTAGGGCATCCCGGCGAGGAGCG |
| | | | | | | 158 | |
| | | | | | 263 | 13414 | CGCCCCGCGTGGCAGAAAGGAGTTCCTCAGCTGCTGTATCTGGTGGGAAA |
| | | | | | | 4055 | |
| | | | | | 264 | 89438 | GGGGCTGTGTGCGGGCCGGGTTCCTCCCTTACCGCTTTGGGTGCCCTGCG |
| | | | | | | 611 | |
| | | | | | 265 | 29293 | ATTCGGATTCAGAAATGCTTTGCAACTCCAGAGCTGTTGAGCCTGCGGCG |
| | | | | | | 325 | |
| | | | | | 266 | 57264 | CTCATTCTGGTTGTTTTCCACCGCGTCTGCTCCCATTTGCGTGGGATGCG |
| | | | | | | 095 | |
| | | | | | 267 | 51487 | AAATTGTGAAATGTTTCCTTTCTGTTTCAAAATAGGCACGACCCACTTCG |
| | | | | | | 529 | |
| | | | | | 268 | 85640 | GGTCCTCTCTTAGGGGCGCGCCGTCTCCTGGCTTCCTGTAAGAAAAGGCG |
| | | | | | | 943 | |
| | | | | | 269 | 38585 | CGCTGGCCCTGTCTGGAATTTAAAGCTCCGTCTGAATTCTGGTGCCTTTT |
| | | | | | | 881 | |
| | | | | | 270 | 13023 | GTGACGGAAGCGGGACTGGAGACATTATGAAGGCTGTTACTGCTCCACCG |
| | | | | | | 5850 | |
| | | | | | 271 | 21653 | TTCTGACAAATCTCTTTGGAATCCTCACTTCATTCAAAGATTCAAGCTCG |
| | | | | | | 473 | |
| | | | | | 272 | 51183 | GGAGGACACCTGGAATCCCTCCTTGCTCCTAAAGGTGTTTCACGGTACCG |
| | | | | | | 988 | |
| | | | | | 273 | 85996 | CGGACCAGACCCAGCCCTCGGAGTCTATTTTTTGCCTCCAGTTACCGGAT |
| | | | | | | 202 | |
| | | | | | 274 | 10335 | CGCCGCACAAGCCCCGGCTTTGTGTTGCTAAGCGATTAGAGCAGATGTAA |
| | | | | | | 5958 | |
| | | | | | 275 | 84140 | CGGATGATCTCCTGCCATGACTCAGCGCTTCTCGCAGGCTGCCCTGCTGG |
| | | | | | | 923 | |
| | | | | | 276 | 37084 | CTGTTAGCCGTTCACCTCGTGCCCGCAGACTTCTCAAAAAATACAAATCG |
| | | | | | | 662 | |
| | | | | | 277 | 37893 | AGTTAGATCTGCAGTTCACCTTAATTTTGGCAGCTCACTGTATCATAACG |
| | | | | | | 981 | |
| | | | | | 278 | 10823 | CGGACCTAGTGAGCCAGGCAGAGCCTTTAGGCCCACGCCTGGGCACCACA |
| | | | | | | 7572 | |
| | | | | | 279 | 10102 | CCCACTTCTTATGCTCTACAAAACAACAGCAGTAGGGGAGTGACTGCACG |
| | | | | | | 278 | |
| | | | | | 280 | 12490 | CGCCAGCTCTCCCTGGGACCAAGCGGTTGGCAAGCGCCTGCAGCCTTGGT |
| | | | | | | 9028 | |
| | | | | | 281 | 15262 | ACAGAAGGCATTTTGGGCTAAGGACGCTCACAACCCGGTTTCCTGTCCCG |
| | | | | | | 2368 | |
| | | | | | 282 | 37673 | TGGCGCCCCCTGTAGTTTATGCCACGCAGCGGTCGCGGGTGTGCTGAACG |
| | | | | | | 335 | |
| | | | | | 283 | 13356 | TTCCGGGGGCTGCCCCACAGAAGCAGGTGGGGATCGACAGTGGCTCTCCG |
| | | | | | | 2101 | |
| | | | | | 284 | 74024 | CGAACGGTGAATCTGCTATGGTAGGGACGGCGCGGACCTTGGAGCCATCG |
| | | | | | | 628 | |
| | | | | | | 42075 | GATGCTGAGCTGAGTTGATTCCCAAATATAAATGTTCTTTTCAGAATCCG |
| | | | | | 285 | 554 | |
| | | | | | | 10335 | CGGCTTTCCGCTTGGCTGGAGACACGCAAACTGAATCTGCAAGTAATGCA |
| | | | | | 286 | 5190 | |
| | | | | | | 20359 | CGGCGGGTTAGGAACTCGCTGGCGTCAGGCTGATTCCAAGGCCCCCAAAC |
| | | | | | 287 | 9089 | |
| | | | | | | 17610 | CGCTCCCCTCGCCCTGCAGCTCGGCTGAGACGCCTCCCTGAGACACCGCC |
| | | | | | 288 | 7427 | |
| | | | | | | 85417 | CGCTTCCCTAACCTGTGTCCAGGCAACAAAGGGGTCCTTGTTTTTGCACA |
| | | | | | 289 | 416 | |
| | | | | | | 17271 | TAGACCCAGGACGGTCTTGTAGGCTCAATGTGTGTGTTTCATCCCAGGCG |
| | | | | | 290 | 322 | |
| | | | | | 291 | 86383 | CGCACTCTCCCAACATTCGGGGAAGGGCGTGCCTTCAGGATTTGGAGTAG |
| | | | | | | 696 | |
| | | | | | 292 | 70945 | ATGAATGGATGCTCCTCCCAGGTGGCACCCAGAGCCTGGCCCCATCCACG |
| | | | | | | 457 | |
| | | | | | 293 | 12953 | CGGACGCACTGCAGAGACGTCCCTGTTTTCGGCGCGTCCTCCGACACCCA |
| | | | | | | 4613 | |
| | | | | | 294 | 33787 | GCGCGGGGCAGTCAGAGTCCCCCTACTTGAGCGGGTTGTGGCGGAGAGCG |
| | | | | | | 739 | |
| | | | | | 7 | 15436 | CGGTGCATCTTACTGAGAACATGTTATTATGCTTTATTCTCGGTACTGAA |
| | | | | | | 3173 | |
| | | | | | 296 | 55382 | TTAGCTGCTGGTGCGGGGCAGGCCTGGGGCAGCGCGGGGCGAGGAAGGCG |
| | | | | | | 901 | |
| | | | | | 297 | 48396 | AGCGTGGAAGGCGGCGGGCTCAGGCGGGCTTTCCGGTCATTAGCGCAGCG |
| | | | | | | 670 | |
| | | | | | 298 | 14022 | TGTTCAAAGTCTTTTCTCTGACAGCATCTGTCTCTAAAGGCCGAACAACG |
| | | | | | | 0810 | |
| | | | | | 299 | 12589 | CGGCTTGGCGAGGGTCTCCTCTGACCCACTTAGCCTTGCCAGCGCTCAGA |
| | | | | | | 9754 | |
| | | | | | 300 | 26250 | AGTACCAGGCCTGTAGCGGTGGGGCTTCTTCACGCCACCGGTGGCTGGCG |
| | | | | | | 744 | |
| | | | | | 301 | 53685 | GGTCAAAAATCCCAGCATCCTTCTGTACCTCTCAAAAGCCCTGTTTTTCG |
| | | | | | | 388 | |
| | | | | | 302 | 11653 | CCCCGCGGTGGGTGTGTGAAAGTCAGGGTGGCAGTGACGCGGCGCCTGCG |
| | | | | | | 278 | |
| | | | | | 303 | 18338 | GCAACTCTGATCCAAATATTTAACTGTGAGTTATAAATCCAGACATCACG |
| | | | | | | 2726 | |
| | | | | | 304 | 14709 | GGCGCGCCTGGGCCAGGCCGAGAACAATGCACGTATTAGGATATCAGCCG |
| | | | | | | 8585 | |
| | | | | | 305 | 38965 | GGAACATGGTGCGGCTTCGGGGCTGCCAGCGGGAAGCCGTTGGGAAGTCG |
| | | | | | | 252 | |
| | | | | | 306 | 12194 | CGCGAAAATCAGTTCCGCCTCTGCCCCAAGAGCCAAGAGGGGTGGAAAGG |
| | | | | | | 6608 | |
| | | | | | 307 | 15468 | CGAGGCCGCTGCTGGGGAGGACGCTGGCCAGCGTGCAGGGGGCGGAGGCG |
| | | | | | | 1197 | |
| | | | | | 308 | 34642 | CTCGCCGTCGTCGAGCGGGGGAATTCTTTCTGTGGGTTTCAAACGCAACG |
| | | | | | | 519 | |
| | | | | | 309 | 17600 | CGCCCTCCTGTCCACCTGCTGGCGCAACCTGCTCCCCCGTCTCTAGGGGT |
| | | | | | | 122 | |
| | | | | | 310 | 35027 | CGGGCCGGTTGTACGCTGTCCACACCGGTTCTCCCACAAACAGCCGCATG |
| | | | | | | 90 | |
| | | | | | 311 | 17073 | GGCGGGCGCCTCCATCCTGGGCGGGCGGAAGGGCTGGGCTGGGCTGGGCG |
| | | | | | | 6335 | |
| | | | | | 312 | 13492 | GGTTGTAGGTTGTGTGTGAATCTGGTCCCTGGCCCCAGAGCTCTGCCTCG |
| | | | | | | 1759 | |
| | | | | | 313 | 10090 | CTCGCCGAATTGCAAGAGAATCTTTAGATCTTGTTCCCACTGTAATCCCG |
| | | | | | | 4316 | |
| | | | | | 314 | 28956 | CGCCACACACCCCGAACCCCCAACACGTTGCTTTCGCACTGATGGGAGCA |
| | | | | | | 577 | |
| | | | | | 315 | 80545 | CGGTCCACGGCCAGGTGAACAATGGTAAGACATGCTGGTCGGTGGCTCCC |
| | | | | | | 125 | |
| | | | | | 316 | 49312 | CAGAGGCGGTGTATAAAACATACGCTGCTTCGGGAACTGCGAGGGGGACG |
| | | | | | | 033 | |
| | | | | | 317 | 15086 | CGGGGCACAGAATCTCCCTACAATACCGTCTTTGGATGGTGGGCATATCT |
| | | | | | | 7810 | |
| | | | | | 318 | 75728 | CGGCTGGGGCAACCCGATTTCCCCACGCCCCCGCACCACGCTCTCCCTTG |
| | | | | | | 458 | |
| | | | | | 319 | 11913 | CGGTTGGATCTTGCCTTTATTGAACATTTTAACAATTTGGTTACTGTGGA |
| | | | | | | 3091 | |
| | | | | | 320 | 27523 | GGGACCGACGTCATGGTTCGGTGCCTCAAGCTGCTGAAGGAATTTAAACG |
| | | | | | | 174 | |
| | | | | | 321 | 39694 | AAGTGCGCGTCCCGTCCGAGTGCTTGAGGTCGGCGGAGGCGGGGCAGGCG |
| | | | | | | 831 | |
| | | | | | 322 | 10119 | AGGAGCAAGAGGACGCGCAGGAGGGCTTCGGTCGCGGTCATCTCTGGGCG |
| | | | | | | 3397 | |
| | | | | | 323 | 46800 | CGCCCCGGGGCGCGCAGTTGGAGGCACATCCCCACCGCACTCTCCACCCT |
| | | | | | | 467 | |
| | | | | | 324 | 49311 | CGCCTCTAGCTGGGCTGTCATTTGTTCTCAGGAGAACACCCCGTCCTTCT |
| | | | | | | 15 | |
| | | | | | 325 | 88791 | CTGAAGAGTCAGAGTAGCACCATCAGATTGGTACCAGTTTTGCCTGAACG |
| | | | | | | 298 | |
| | | | | | 326 | 58357 | AGGCAGGTGTCAAGCTGGGGCTCTTCTTATTGGACGTGAGGGCCGAGGCG |
| | | | | | | 987 | |
| | | | | | 327 | 15726 | CGCGCCTAGGTTTTCGCAGCTGGTTTCTGTCGGTGGGTTGTTAATAGGCT |
| | | | | | | 821 | |
| | | | | | 328 | 26271 | AGTTTTTCTTTCAGGTAGTCTGAGATGGCCCGCACCAAGCAGACTGCACG |
| | | | | | | 587 | |
| | | | | | 329 | 32110 | CGCCCCCAGACTCCGACTTAGAGCGGACTCCGATCGCGGCAAACCATGGC |
| | | | | | | 145 | |
| | | | | | 330 | 32596 | ATGTAAGGGATATTCCCCTTTTCCCTTTGGAGTGGTATCCCTAGGACACG |
| | | | | | | 886 | |
| | | | | | 331 | 19743 | GTAGTTGTGGGTGAAGCAAACCCTAATTCCAGAAGTTTTTTTCCCTAGCG |
| | | | | | | 759 | |
| | | | | | 332 | 15089 | GAGCCAATGAGGCGGGAAAGTGGCTGCGAGAGCGGCTGCCAACTCGGCCG |
| | | | | | | 6413 | |
| | | | | | 333 | 13421 | CGCCACACGCCCCGACCCCAGAACATGACCACCCGTCAGAGCCGGGTACT |
| | | | | | | 61 | |
| | | | | | 334 | 35078 | TGTGTCAGGGGCCCGTGTGTTTTGGCGACACCCGATTGTCATTTCCTTCG |
| | | | | | | 67 | |
| | | | | | 335 | 11372 | TTCGTTTGAAATGGCCTCTGTCAGCGCATCTTGCGCGAAATAAAACACCG |
| | | | | | | 2758 | |
| | | | | | 336 | 10184 | CGGAGAGGCATAAAAGCCATCACCCAGAAGCAAATGTGTTTGGGCTCCTT |
| | | | | | | 1158 | |
| | | | | | 337 | 10131 | CGCGGGAGTGGCCAAGCTACCCAACAGGCTTCACACCACTCGTTCATGCA |
| | | | | | | 7622 | |
| | | | | | 338 | 97831 | CGCGCTCGACTCCCGCCCCACCTGTGCCTTGGCGCCCTCGCTCACCGCCC |
| | | | | | | 98 | |
| | | | | | 339 | 51290 | CTAAGATCTGCTGAGAATCCCCAAGGTGCTCCAGGATTTTCAGTGTTGCG |
| | | | | | | 751 | |
| | | | | | 340 | 11513 | CGTTAGGAAGATCTAAACTGAAGCACTAGAAAGGTCTGTGACTCTGTTCT |
| | | | | | | 1789 | |
| | | | | | 341 | 85671 | CTTGCGTGAGCAGTAGCCTCAGCTCTGCGTTCTTTGGACACTTTCCAGCG |
| | | | | | | 811 | |
| | | | | | 342 | 91687 | CGGCAGCTGTATCTTCTGGGATGGATTCTTATAGCACCTATTTGGCAACT |
| | | | | | | 463 | |
| | | | | | 343 | 45008 | GAAATGGTTTAATTCATGCTCAGGAGCTGTGTGCCTTTCCATCCCTTCCG |
| | | | | | | 447 | |
| | | | | | 344 | 31637 | CGCTGCTCAGAAAGAAAACCTCACTGATAGGACCTCCACCCAGACCTCTT |
| | | | | | | 55 | |
| | | | | | 345 | 12344 | TGGTAGTAAAGACAGCGGCTGTTTATTAAACTCTGCTTATCTCAGCTTCG |
| | | | | | | 6272 | |
| | | | | | 346 | 75958 | CGGGCTGACACAAATAGGCTTCTTCAAGAGTTGAGGAAGAAAACAAAATC |
| | | | | | | 299 | |
| | | | | | 347 | 42688 | TCCCGCTTTTGCTAAGCGTGGCGGGTCCCAGGCAGGGTTGGGGAGCGACG |
| | | | | | | 886 | |
| | | | | | 348 | 36628 | CGGCGCTGGACTCCCTCCAGGTCACACCGCCTGAGGTCACACCGTCTGGG |
| | | | | | | 713 | |
| | | | | | 349 | 20359 | CACAGAGAAACTGCTGAGGTTTCTCTGCAAAGCTAAAGAAATTTAATTCG |
| | | | | | | 6940 | |
| | | | | | 350 | 24281 | AATCTGGTTGCCATGGAGACCATGCTGTTGTCAAGGTGGCTGATTGGCCG |
| | | | | | | 1830 | |
| | | | | | 351 | 43393 | CGCCCTCCGTCCCACGGTTCTGCTCCGCAGGGAGGCTCGGCCCCATCTCT |
| | | | | | | 728 | |
| | | | | | 352 | 20548 | TCCGGAAGGCAAATTATCTTTCCACACCAGAAAGGTTTATAAACAAGCCG |
| | | | | | | 412 | |
| | | | | | 353 | 30661 | CGGAGCCACAGGCCGCACCAGCCCGTCTTTGATCCCGGGTGTTCGGACTC |
| | | | | | | 534 | |
| | | | | | 354 | 16642 | CCGCGCGCTTCATTTTTATCAGGCTCCTGGGTGTGTGCAGGGAGGGAACG |
| | | | | | | 2671 | |
| | | | | | 355 | 16491 | CGCTTTCAGAGGAATTTTTTTAAGGCTCAGCTGACAGCACTACAGTCCTG |
| | | | | | | 1744 | |
| | | | | | 356 | 36040 | CGCCGGAATCCCACCAGCCTGCAGCCACGGTAGGCAAGAGCCTCGGGGAA |
| | | | | | | 903 | |
| | | | | | 357 | 12899 | TGTCACTTACCTAGAGATGTTCTCACGAGAGCTTGCGCCTACCAGGATCG |
| | | | | | | 8642 | |
| | | | | | 358 | 25241 | TACCGGCCGGAGGACACGGGGGGACGCTGGGAACTCGGGGTGAAGGGAC |
| | | | | | | 260 | G |
| | | | | | 359 | 26273 | CAAGTACACCAGCTCCAAGTAAACTAGTTACCTGGGTAAAAGCGCTAACG |
| | | | | | | 611 | |
| | | | | | 360 | 15802 | GTAATGTCCTCATAGATTGCTTGTATCTTCAGCTGCGTCTGTGGCCTGCG |
| | | | | | | 7914 | |
| | | | | | 361 | 23847 | CGCCCGCAAAGGCGCCCTCCGGCAGAAGAACGTGCATGAGGTCAAGAACC |
| | | | | | | 556 | |
| | | | | | 362 | 75315 | GAAAAGTGTTTTGTCTGAAGAGCAGAACTGTCTTATACCAGCCCTGTGCG |
| | | | | | | 081 | |
| | | | | | 363 | 11271 | CGGGCGGGCTGAAGCCCCCGCGCAAGTTCGAGACACACTTACAGTTAGTT |
| | | | | | | 1380 | |
| | | | | | 364 | 14021 | CTTTCTATTGACTAATTGTCAAGACATTAGACTTCCATCTGGGAGATACG |
| | | | | | | 9613 | |
| | | | | | 365 | 12201 | TCCCTTCAGTGCTTCCGAAGACACCGGAGGGGAGTACCCCGCGTAGAGCG |
| | | | | | | 7052 | |
| | | | | | 366 | 10299 | TGGCCTTAGCCCGGACTGTGAGTCTCGAGCGGCTTCCGAGAGGCAGCTCG |
| | | | | | | 7382 | |
| | | | | | 367 | 20001 | CGGCCTCACCCAGCGACCCCAAGGGTAGGCCCTCTCCGGAGCTGCCGGCT |
| | | | | | | 0283 | |
| | | | | | 368 | 15408 | CCCTTCTGACGACTGTACGATCTGATGATAAATCATGTGTTTGAGACTCG |
| | | | | | | 7975 | |
| | | | | | 369 | 51973 | CGGCGACGTACACTGCTGCTTAGGGAGCAAAGCACTTCCACCGCTTTCAC |
| | | | | | | 27 | |
| | | | | | 370 | 14107 | TAATATCTATTAGGAGCTTCTCAGCAGGACCAGGACTTTAAGGACTTTCG |
| | | | | | | 1438 | |
| | | | | | 371 | 10090 | ACAGGTTAATAAACAACCTGTCCGGCACAGGTTAACAGGAGCCTCTCTCG |
| | | | | | | 3909 | |
| | | | | | 372 | 32116 | CAGAGGTGGCCACCCCTGTTCCCCTCCTGGCACCATGATGGTCAGGACCG |
| | | | | | | 56 | |
| | | | | | 373 | 51568 | CTAGTGATCGCCTCCCTGACCTTGGCCTTGTCAGGAGGTAAGAATGCGCG |
| | | | | | | 260 | |
| | | | | | 374 | 42387 | GCTGCTCCCTGGAGCTTGCTCAGGTCAGGTGAGGAGGATACACTAGCCCG |
| | | | | | | 193 | |
| | | | | | 375 | 11958 | CGCCTAAGCCTGGCAGAGGCAAAGCGCTAGACGGAGCACCGCAAAAGCCC |
| | | | | | | 326 | |
| | | | | | 376 | 94452 | CGCAGAAATATCTCTCTCCGCCCGAGAGGAAGCGTCTGGCCAAGATGCTG |
| | | | | | | 236 | |
| | | | | | 377 | 75316 | CGGATGCATTCGTGATTGCAACAGATTGAAGAAATTAGACCAGACAAAGA |
| | | | | | | 233 | |
| | | | | | 378 | 13366 | ATATTATTTATATAGATTGTCCTCCCACTCGTCCTCAGCCTATAGGAACG |
| | | | | | | 2267 | |
| | | | | | 379 | 96148 | CCCTCTCCAGAAATCAATTAATTTCTCCCATGGTATTACTGTAAATATCG |
| | | | | | | 290 | |
| | | | | | 380 | 72756 | AGAGCCGAGATTTGGGCTCTGAAGCCCGACGGCCCTTCAACATGTCTGCG |
| | | | | | | 882 | |
| | | | | | 381 | 21926 | CGCGCATCCGAGACCGCCTCCCCAAAGCCCGCTTCATCGCCAAGAGCGGG |
| | | | | | | 476 | |
| | | | | | 382 | 10091 | GACCCCTCCCCGCAAAAGGCAAAATCAAGACTACAGAAGGTGAACTTTCG |
| | | | | | | 5805 | |
| | | | | | 383 | 78176 | CGGTGAGCTGGCTGCGGAGGAACCAAAGAAAAATGAATCCTCGAGGAACC |
| | | | | | | 806 | |
| | | | | | 384 | 13392 | CCTCTGCGGCAGAGAACTGCTTCCCGGTGAGCTCGTGGTGGGTGGAGGCG |
| | | | | | | 131 | |
| | | | | | 385 | 10099 | ACCATGCCGTTGTACCGTCTGGGTACAGAGCAAAGGGTCTCGTTTTTACG |
| | | | | | | 6070 | |
| | | | | | 386 | 38624 | GGCCTGCAGGCGCGACCTGAGGCCGCCAGGGAGGCTGGGTGGCGAGGGCG |
| | | | | | | 23 | |
| | | | | | 387 | 46951 | CTGGGTGGTGGGCGGCTTAGACCTAATTCCGGAGTTAATGGCGGCGGCCG |
| | | | | | | 477 | |
| | | | | | 388 | 49893 | CACGGGGCTGCCCTTTTCCACCCCTAGGGGCCTGCTGAAGACTGAGGACG |
| | | | | | | 122 | |
| | | | | | 389 | 13412 | CCTTGGGCTCTGGGCACTATGTGGGAGGCAGCATCAGGTCCATGGCGGCG |
| | | | | | | 5181 | |
| | | | | | 390 | 51139 | CGCGAGTTCGGAGCAGCGCGTGTATCTGTGGGTCTTTGGGGCTGGCCTCG |
| | | | | | | 066 | |
| | | | | | 391 | 73578 | CGGCAGGCCTGATTGGTGGGCTTTTAAACGTGACTAATGAAGAAAATTTA |
| | | | | | | 307 | |
| | | | | | 392 | 95437 | AATTAAACCCATAACCAAGGAATATACTAGGTTCAACTATGTACTTAACG |
| | | | | | | 294 | |
| | | | | | 393 | 10622 | TCAGCAAAATAAAGGATTCACTGCAATTCATCCTTCTGTGGTCACTGCCG |
| | | | | | | 7786 | |
| | | | | | 394 | 13702 | TTTTTGGACTGGAAGGCGGGGAACCTGATCCTGCCTTTGACTCAATTACG |
| | | | | | | 8442 | |
| | | | | | 395 | 68781 | CGCCATCTGATTGCTATTAGGGCTGGGGAAAAGGGAATTCCTGGTTCCAT |
| | | | | | | 279 | |
| | | | | | 396 | 28550 | CGGCAAATAGCGACATGCTCTCGGTTTCTGTCGGCCTTGGCCTGGCAGAG |
| | | | | | | 046 | |
| | | | | | 397 | 68924 | CGGCCACCTCCTCCCGACGCTTTCAGCAGCCCACGCGGCTCTTCCGGCCC |
| | | | | | | 853 | |
| | | | | | 398 | 31021 | ACAGCAGTGAGGTTAGAACGTGAGGGGATCACAGACTTCGTCCACCTTCG |
| | | | | | | 003 | |
| | | | | | 399 | 62584 | AAATGTAAACCTTAGGATTGGTTCTTGGGTGAAGAGTCCGCTGCTCTTCG |
| | | | | | | 513 | |
| | | | | | 400 | 15811 | CGCGGAAGCCTAACCCCCAGGTGCTCCTCGATGACGGCTTCGTGCCTTCT |
| | | | | | | 4659 | |
| | | | | | 401 | 85997 | CGGGGCTCGCTCCCGCACTTTGAAGGCTGCCGCGGTCTTTCGTCATTTAT |
| | | | | | | 753 | |
| | | | | | 402 | 19915 | CGCACCCATCACCTAACCGTGTCTCTGGTCACCTGTGGGTTTCAGCGGGT |
| | | | | | | 874 | |
| | | | | | 403 | 10357 | CGGCCACACCGCTGGAGTCCTGGGCTGGCAGCGGTAACCTTATCCTTGTG |
| | | | | | | 4523 | |
| | | | | | 404 | 21961 | GAGGACCTCGCCGCTCTGAAGAGCACCGTGCACATGTGGGTGCACAAACG |
| | | | | | | 713 | |
| | | | | | 405 | 46969 | CGGCTCCATGGTTCTCATGCCCCAAAGTCCTGCAAATTGACACGTAAGTA |
| | | | | | | 673 | |
| | | | | | 406 | 23243 | TCAGAGGACTGAACAACACCAGAGGCGTGAAGCTTGGTGCACGGCAGCCG |
| | | | | | | 96 | |
| | | | | | 407 | 14919 | CGGCACTCATAGATCCGCACGTCCCGAGAAGCCTCTGCCATCCCAACCCG |
| | | | | | | 2846 | |
| | | | | | 408 | 85405 | ATGGCTGGACTTGCTGCGTGGGTGATCCCAGGACTAAGGCAATTAGAACG |
| | | | | | | 132 | |
| | | | | | 409 | 47483 | GGCTCCTCCAGGGTTCCGGCTGGTGGGAATCTAGGGAGGTCCAGGAGACG |
| | | | | | | 257 | |
| | | | | | 410 | 75315 | CGGCTAGAGACTCACTGACTCATGCGTGTGTGGTAGGAATCTTCCAGGAA |
| | | | | | | 503 | |
| | | | | | 411 | 14062 | CGTTTCGAAGCAAAGACAACGCATGGTGGCGCTGCAGGCTAAGGCTTCAA |
| | | | | | | 4869 | |
| | | | | | 412 | 22028 | TCAGAAACTCCTGGTTCACCGCGTCGGCCAGTGAGAAGTCCAGCAGCTCG |
| | | | | | | 3442 | |
| | | | | | 413 | 11906 | TAATACAATAACAGAGGCCTCCGCTCCATTTGCATTAGGTGAGGAGCGCG |
| | | | | | | 7552 | |
| | | | | | 414 | 13099 | CGCCGGGGCCCCGCCCAGGCCCTAACGAATACATCCCATTGGCAGGAACC |
| | | | | | | 6123 | |
| | | | | | 415 | 55047 | CGCCCAGCCTCGCTGTTCTTATCTTGGCAGCAGATTCCGAATGTCGGCTG |
| | | | | | | 721 | |
| | | | | | 416 | 60386 | CGCCCAGTGAGGTCCAGAGAAGAGAGGGACCTTCCGAGGGTCTTCTAGTC |
| | | | | | | 272 | |
| | | | | | 417 | 19788 | TCTCCTTAGAGGGACTGAGTCGGGATGCCAGAAACCCTGAGATTCTCTCG |
| | | | | | | 7649 | |
| | | | | | 418 | 33701 | CTGCGGCTGAGGCAGGCTCCGCAGACCCCGAGCCAGAGTGGGATTTAACG |
| | | | | | | 350 | |
| | | | | | 419 | 56941 | GATGTGCTGCCGGGCTCAGGTCCCGCAGGAGACCCACCTGGAGTTCCTCG |
| | | | | | | 473 | |
| | | | | | 420 | 10187 | CGGCCCCTGAACTTTTGCCTGCACCGGGGCCGCCCCGCACCCCCCGCCAA |
| | | | | | | 5035 | |
| | | | | | 421 | 50557 | CGTAGAGCTGGAGGGCGATGGTGGCGGAGCTGCTGGGGGCGGAGGCAACG |
| | | | | | | 066 | |
| | | | | | 422 | 15162 | CGCATTTGGGGACTCACCTTTGGAGAACTTTTGAAATGTGGCAGTTTAGT |
| | | | | | | 0975 | |
| | | | | | 423 | 46922 | CGCCCCCTCACCAGCGCCTAGCCAGTCCGTGTCCCTCCGCCTCCAGGAGG |
| | | | | | | 87 | |
| | | | | | 424 | 60027 | CGACCCCGCCCTGCCGCCCACTGCCCTTTCCTGCGAGACCCTGGCTCCGC |
| | | | | | | 66 | |
| | | | | | 425 | 62660 | CGCGCCCTTCGCGCCTGGACCAGCCTTATCCGGGGCACCCTGGACCTCAC |
| | | | | | | 861 | |
| | | | | | 426 | 84628 | ACGTTATGCTTACAAATGTTTGCTGCTAGAGATCTGGCACGACAGTGACG |
| | | | | | | 969 | |
| | | | | | 427 | 47696 | CGGCGAGCGCTGCTCAGCTTCAAGCCTGGAGGAGCCTCCCACTCACCAAC |
| | | | | | | 788 | |
| | | | | | 428 | 26273 | CGTTTGGGCTCACCAGCATTTTCCTGTGGTCATTTGACGGTATCACTTCG |
| | | | | | | 193 | |
| | | | | | 429 | 68172 | CGGCTGCCCTAAAATGAGAGTTTGTCTTTGAGTTGGATCATGACTCTGTT |
| | | | | | | 80 | |
| | | | | | 430 | 13251 | CTTGGAAATGACAAACACTCCTCGTGGTCGTCTGATCCCAGATCATTGCG |
| | | | | | | 3895 | |
| | | | | | 431 | 88717 | GAGGAGTTCTGAGACCCCCAGTAATTCCCTTGCAGACCCTGCGGAGCGCG |
| | | | | | | 134 | |
| | | | | | 432 | 67069 | AGTAGGCTGGGAGGCTGCAAGAGCGAGTGCGGAGCTGGGTGACAGCAACG |
| | | | | | | 959 | |
| | | | | | 433 | 14008 | AGCCCGCTGTGTGCAGGGTGCAGCAGTGACAAGCAACAGTGCTGGTGTCG |
| | | | | | | 1972 | |
| | | | | | 434 | 44654 | AATGGGATCTAGTGCTTCTTACCTGATGGACAGGTTCTATACCATTATCG |
| | | | | | | 206 | |
| | | | | | 435 | 10089 | CGGAACTTTGTGAACAGCTGGGGACTATGCTTAAAGACAAAGTCTGACTG |
| | | | | | | 7081 | |
| | | | | | 436 | 29976 | CGGAGGAGCTGACTACCAGAAGCCCCCAGTGGGTGTTAAATCACAAGCTA |
| | | | | | | 613 | |
| | | | | | 437 | 18306 | CGGTCCCATTATCCCGAGCACGACCGGCTGCGTTCACACCGCAAAGTCAC |
| | | | | | | 2301 | |
| | | | | | 438 | 18141 | CGGGTGCTTTTAAATACACCTGGGTATTATGAGCATGTAATCTGGCCTAA |
| | | | | | | 3810 | |
| | | | | | 439 | 16532 | CAGAATCTCTGGCTTCAGCTCTGCAAGACCCGTGTGTGGACACTCTTCCG |
| | | | | | | 4100 | |
| | | | | | 440 | 13414 | CGCGTTCCTTGTTGAGTCATTTCCTGTCTTGATTTTAGGACCTTCCTCCG |
| | | | | | | 3489 | |
| | | | | | 441 | 11529 | CTTTGGGTTCGAAGAGACAGACCCCACGGAAGATGCCTCCAGGGTTGGCG |
| | | | | | | 7989 | |
| | | | | | 442 | 23859 | CGCGCCTGTGAGCACTCACCTTCCCGCCGTGACCCGGGCCCAGGGCTTCA |
| | | | | | | 9734 | |
| | | | | | 443 | 58414 | GTACATGGCACTTGAGGTTCACTTTGTCCAAACCAGTACCTCTTATCTCG |
| | | | | | | 82 | |
| | | | | | 444 | 14711 | CGCTGTGACCGCCACAAAGTGAATCCTTTCGGTGCGGACAGTCGCCTTCA |
| | | | | | | 1660 | |
| | | | | | 445 | 21490 | CGCAGCAGCCCAGGGACGCGATGACACTTTATTCCCGCGTTCCACACCGC |
| | | | | | | 238 | |
| | | | | | 446 | 13663 | ACCTGGCAGGGCTGGGGTGGGGATTGCAGCTAGTCCATGACCCAACTACG |
| | | | | | | 0705 | |
| | | | | | 447 | 37390 | CACTCACACACAGTAAGCTGGGTCCGGGGGGTCAGTTTACCCATAGATCG |
| | | | | | | 176 | |
| | | | | | 448 | 45179 | CGGCGCCAGTGTGTCCCGGGCGAGGTAGGGGCAGGCAAAAGGCGGCTGCG |
| | | | | | | 787 | |
| | | | | | 449 | 15479 | TGTAGTCACCTGATTAACAAGAGCCAGAGTTCATTGCATGCTGATCAACG |
| | | | | | | 6431 | |
| | | | | | 450 | 36919 | CGCAATAATGCTGCGTCACGAAACTGCGCATTCCAAGAGAAACTTTTCTC |
| | | | | | | 738 | |
| | | | | | 451 | 12194 | CGCTGAGGCAGCGGCAGACTAGAGCTCCTGGTCCGAGTTCGTCTGCAGAC |
| | | | | | | 2010 | |
| | | | | | 452 | 15727 | GCCCCGGGCAGAATAGGTGACTACAGATATTCTCCTGATGCGACAGGTCG |
| | | | | | | 525 | |
| | | | | | 453 | 38214 | TCAGAGCCTCTGATTCTGCAGAACGCAGACTTGCAGCTGGTCCACGGCCG |
| | | | | | | 979 | |
| | | | | | 454 | 17519 | GGACTGGGGACTGCCCCCAACTTCCGAGCGGCAGAATCTGGCGCTTGGCG |
| | | | | | | 1374 | |
| | | | | | 455 | 23900 | CAACCTCGCTGAGTTGGTGCAGCTCCAGGTCATTCCTGGGGTGGGAATCG |
| | | | | | | 7828 | |
| | | | | | 456 | 19212 | TTTCACACATTTTGTCGCCACTGCCCAGACTTTGACTAACCTTGTGAGCG |
| | | | | | | 6165 | |
| | | | | | 457 | 80981 | CGCCCAGCCCCCAGCTCTTTGTTCTGGCGCAGCCTTTAGCCTCAGAGCGC |
| | | | | | | 129 | |
| | | | | | 458 | 25102 | CGGATACGCACCCACAGTGGCTGATTCGGGGGTAACCGTGTCATTTGCTT |
| | | | | | | 274 | |
| | | | | | 459 | 20001 | GCAGCCGAAGTAGGCGTTTGGGGAGGCGAACTCATCGGGGCAAAGAAGCG |
| | | | | | | 0788 | |
| | | | | | 460 | 67085 | CGGAACATGTTTGCTTATTTATCCATAGTTCTCCTAGGAAGGACGGTGTT |
| | | | | | | 823 | |
| | | | | | 461 | 22510 | CGTGGCCGCTCTCAACTCCTCCAATTGCGGGTTCCAGGCCATCCGCGGAA |
| | | | | | | 61 | |
| | | | | | 462 | 60289 | CGCCCCCTAGCCATTCTTAGCTGGGGGAGGCCAGCACGGATCAGCCGCTT |
| | | | | | | 532 | |
| | | | | | 463 | 16554 | CGCCTCCACCCTGCCCCACCCAGGCCGCGCGCACCCCGGAAACCTGGACT |
| | | | | | | 466 | |
| | | | | | 464 | 16691 | CGCCTGCAAGACCGCATTTGTTCTCTTAACACAGGGCAGTGAACGGCCGC |
| | | | | | | 7158 | |
| | | | | | 465 | 51190 | CGGAAAGTCAAAATCGCAAGCACTTGCAGTCGCTAGCGAAGTCGGTTTCT |
| | | | | | | 201 | |
| | | | | | 466 | 10072 | ACAGGTAGAAGACCAAAAGGCCAATGAGACTTCTTTCCTATTTGAGATCG |
| | | | | | | 2584 | |
| | | | | | 467 | 45250 | TCTGCTCAGGGTCGTCGCTCATGCTGCGGGCGGGAGGGCGGGCTGAGCCG |
| | | | | | | 181 | |
| | | | | | 468 | 19813 | TACGTCAACGCGGGCTCATTCTGAGAAAAACTTTCTTTAGCTTGTACGCG |
| | | | | | | 299 | |
| | | | | | 469 | 37123 | ATGAGACGGAGGGCACCATGGGAGCTGGCTCTCTTGCTATTCATAAAGCG |
| | | | | | | 843 | |
| | | | | | 470 | 18963 | CTTTCAGGTTCCAGGGCACTCGCCACTTTTTCCAAAGCCATCAGTCTGCG |
| | | | | | | 958 | |
| | | | | | 471 | 32459 | CTCTTTAGTCGGAGAAGTCCAAGTCGGGCGAGGGGGCACCCCGGGGTTCG |
| | | | | | | 428 | |
| | | | | | 472 | 26190 | TGCGTTGGGAAGGCATGGGGTGCGGAACCCGGCCTGACCTCGCTGTCCCG |
| | | | | | | 328 | |
| | | | | | 473 | 27806 | TTCAGAGGTCCCTAGCACTCACTCGTGTGCTTGTGATTTGCTAGACTTCG |
| | | | | | | 529 | |
| | | | | | 474 | 11671 | CCCTCGCTGGGGCACCTGGGGAGGGGGCGCTGGGGCACCTGGAGAGGGCG |
| | | | | | | 0943 | |
| | | | | | 475 | 54971 | CGCAGTGATCCCGTGACCCCTGTTGCTAGACATCCAGAGGCTTCTGGTCC |
| | | | | | | 018 | |
| | | | | | 476 | 10048 | TGCTGAGCCCCCGCCGTGTACGGGGGCCATTGTGGGGCCTTGGGGTCTCG |
| | | | | | | 8942 | |
| | | | | | 477 | 89949 | CGGAGAAAACACTTCACCCTGGAACCCTAACTCAGGTCCTGGCAAAAGAT |
| | | | | | | 694 | |
| | | | | | 478 | 12293 | TATTTAAAGCTCAGAATATGATAAAGGTCCTGTCTCCACCACTTCAAACG |
| | | | | | | 5222 | |
| | | | | | 479 | 15086 | GTGGGCGGGGACAGGGCCCCTGCTGGAAGAGGCGCAGCAGCGAGGTGGC |
| | | | | | | 3969 | G |
| | | | | | 480 | 85424 | CGCCGTAGGATCTTGCGTTCGGCGCTCTCCCAGTGGAGGCCAACACCAAA |
| | | | | | | 288 | |
| | | | | | 481 | 45914 | AGGGGTCGGGGGCTCACACGCCCTGCGATTTCCTCCCATGAGAATGGGCG |
| | | | | | | 450 | |
| | | | | | 482 | 11867 | CGATCCTTGGGCTATAAGCCATCTGACTTCAACTATCACTGCTGTGTTTG |
| | | | | | | 7172 | |
| | | | | | 483 | 11216 | CGCCGCCCCGCAGGAGCACGCACTCCCACACAAAGCGCCTGCAGCTGACA |
| | | | | | | 57 | |
| | | | | | 484 | 38068 | ACAAGGGGTCACACGTGCGTGTGCCTGGCCTTCGCAGGTGGATGTGTGCG |
| | | | | | | 340 | |
| | | | | | 485 | 15268 | CGCCCACTAATTCCTTTGGTGCTGCTCCTGGTCCAGGTCAGCAACAGCCC |
| | | | | | | 1887 | |
| | | | | | 486 | 15156 | CGTGGTTTAACAGTCAAATATTAGTGTTAGTTGCTCATGCACCATGATTG |
| | | | | | | 2205 | |
| | | | | | 487 | 25908 | CGGATTTCAAGAAGTCTTTGACGGTGCAAACTGGACTTCTAATCATGGGA |
| | | | | | | 279 | |
| | | | | | 488 | 15852 | ACGCAGTGCGTGTCAGGTAGGAGTAAATGCTTATAGAGTTTCATCTCGCG |
| | | | | | | 4404 | |
| | | | | | 489 | 70014 | GCAGCGTCAGTCGTCTGCAGGAATTTGTGCTGGGGTAAATGCGCGCTTCG |
| | | | | | | 873 | |
| | | | | | 490 | 44163 | ACTTAGGAAACAGGCCCAGGGAAGTAGCAGAACAGTAGCGCTAGAGGAC |
| | | | | | | 975 | G |
| | | | | | 491 | 46143 | CGGCCCCGCCGGCCTCGCACCCGGTAATTGCAGCTTCCCCCCGGGGCCCT |
| | | | | | | 498 | |
| | | | | | 492 | 28839 | CGCGGGCTGCTACAGAATCCCAGCCGGGCTTCTACCATCCAAACCCCTTT |
| | | | | | | 249 | |
| | | | | | 493 | 18254 | TCAAGAACATCTGACTCCACCATAATTCTCCTGTATTGCACAGGATGTCG |
| | | | | | | 7599 | |
| | | | | | 494 | 10717 | CGCGCCCCCAGCTTCGCGGCAGCTAGGTCCCTCCCCAGCGCGCTGAGCTC |
| | | | | | | 9321 | |
| | | | | | 495 | 96180 | ATCCTGGTGTCGACTGTGAGTTCCCAGCAGAGGCCCAGAGTCCCGGTCCG |
| | | | | | | 842 | |
| | | | | | 496 | 42445 | CGCCTGGCCCCGATCAGTCCCCACCAGGACCACCTATTTCCCCAAGTCGG |
| | | | | | | 244 | |
| | | | | | 497 | 36983 | CTCCTCGCCGTCTACAGTCGGCATAGTTTGAAGTCTGTTTACTTGTCCCG |
| | | | | | | 694 | |
| | | | | | 498 | 15836 | CGATGTCAGACATTTCATGTCCTGTCCATGTGGAAGACTGCATCCTTGAC |
| | | | | | | 1858 | |
| | | | | | 499 | 81106 | TGAAGTGGAGCAGACTCACCGGCAAGGAGCCCGGAAAATCGTCAGGGGCG |
| | | | | | | 663 | |
| | | | | | 500 | 91295 | GGTGGCTCAGGTGACACGGGCATCCTTCAAGGAAGGGCGGCGGGACGGCG |
| | | | | | | 159 | |
| | | | | | 501 | 19788 | AATCCTCCTTGCAGTAAATGCTACCGTCCTTGGCAAAGCAGGTGAGCTCG |
| | | | | | | 9248 | |
| | | | | | 502 | 20145 | ATAAGACCATCAAAAGCAATGTCCCAGGACTTACAATGTTTGCTAAGACG |
| | | | | | | 0506 | |
| | | | | | 503 | 48056 | CGTCGCCAGTGTGGGAGATCATTTATAATCTTGATGGATACAGGTATAGC |
| | | | | | | 973 | |
| | | | | | 504 | 13296 | TGTGGGCTGTGCCAGAACCCTGGGAAGAGCAACGTGGTCGGAGGGTCCCG |
| | | | | | | 9187 | |
| | | | | | 505 | 11033 | CGGTTTCGATTTGGGACACCACCTGTCAGCCAGGCAGGTTAAACTGACGG |
| | | | | | | 9005 | |
| | | | | | 506 | 81189 | CGCACCAGCGAGTCCCAGGGCCTCCCTCACACTCCCGAGAGGAATCTATT |
| | | | | | | 927 | |
| | | | | | 507 | 38942 | CGGCGCCCCCTCATTAGAGGGCGTTTGTTAGGGAATGTTTTCCCAGCGAT |
| | | | | | | 313 | |
| | | | | | 508 | 10848 | CGACCCAGTCCTGACCCGGCCCGCTGCGCGCCGACCTCCTAGCTGCCCCG |
| | | | | | | 9977 | |
| | | | | | 509 | 38036 | GGGGCAGTCTTGGGATGGCCATCGTCCACATCCCTGAATGGGGCAAGCCG |
| | | | | | | 060 | |
| | | | | | 510 | 69223 | CGGGGGCCTAAAAAGACTGCTTACATACACCACCTTGGATCTTTCAGATT |
| | | | | | | 20 | |
| | | | | | 511 | 75315 | CGTCAGGAATTTGACTCTGTGAGTTGGTTTCCAAGAGTCTAAGTTAAGCA |
| | | | | | | 868 | |
| | | | | | 512 | 78933 | CAGGGGGCGTCGGGGCGGAGCAGCGGGCCCAGTACATCCCTGAGTGGGCG |
| | | | | | | 630 | |
| | | | | | 513 | 17263 | CGAGTGTGGCCTTTCAAAAAGAAGTGCATTCTAATTTTAAGTTGAAGTTC |
| | | | | | | 5656 | |
| | | | | | 514 | 12876 | TGTATTCTTGGGGTTCGGGCTGTATTCCTGGACCCCGGCTCCCGGGTCCG |
| | | | | | | 4824 | |
| | | | | | 515 | 13421 | CGACAAATACGAGAACGGGTACATTCACCCGGTCAACCTGGTGAGTGCTC |
| | | | | | | 0946 | |
| | | | | | 516 | 88326 | CGCTGATGTGCTTTAATAGCGTGGTAGTGAAGTCCACCATCAAAGCAAAT |
| | | | | | | 918 | |
| | | | | | 517 | 10185 | GCCTTTGATCCTGTGTTGGGACAACTTGGGTTTTCCTGGGTGCCCACCCG |
| | | | | | | 0540 | |
| | | | | | 518 | 36982 | CCCTGAATTACGTACACAGCTAGCAATTAAAAATTAACCTGTGGTCCCCG |
| | | | | | | 194 | |
| | | | | | 519 | 44325 | CGTCCGGGTGCAGGACGGCGCTCTTACCGCCCCACCCCAAACCGTTGCCT |
| | | | | | | 823 | |
| | | | | | 520 | 51184 | TGTTCCGGAGACGGTGCAAACGGACGGGGAAAGTGTCGGGGTCTGGCTCG |
| | | | | | | 355 | |
| | | | | | 521 | 10139 | CGGGAGGCAGCAGAGCCATGGCGGCCACCCGCCTCTCACAGGGACCCAGA |
| | | | | | | 90 | |
| | | | | | 522 | 10540 | CGGAGTGATCTTCTGCATCAATCTAACATCTTGATTTTGTGCGATCACAA |
| | | | | | | 5068 | |
| | | | | | 523 | 15026 | CGCCCGGCCCAGAACTTCATTTTTAAGATAATAAACTGAGGGGCTGAGAC |
| | | | | | | 7044 | |
| | | | | | 524 | 79815 | TCCGAGACCTCAGGGCGCGTGGCAACTGGGCAGAAGCCGTCTCTGCAGCG |
| | | | | | | 718 | |
| | | | | | 525 | 91171 | CGCTTTAAAGTGGCTACAGCTCTGATGCACCCTACTTCCAGTGGGAAACT |
| | | | | | | 013 | |
| | | | | | 526 | 15830 | CTTTCCTAGGTGCAATTGGACACTAAGGGTCTGGGTCTTTCTGTCTCCCG |
| | | | | | | 1038 | |
| | | | | | 527 | 44332 | CGCCCCACGTCTGCGCCCAGGCGAGGGGATTTCCTGCCAGGCGAAGTACA |
| | | | | | | 664 | |
| | | | | | 528 | 57358 | ACAGCTTTTAGGTAGACGTGGAGGCGACTCAGATCGCCTCGCGGTTCCCG |
| | | | | | | 713 | |
| | | | | | 529 | 27015 | GAGGAGTTCCAGTCACCGAGCGAGGGGCGCAAGGGTGGGTGCATCCTGCG |
| | | | | | | 872 | |
| | | | | | 530 | 36604 | GTAATAAACACCAGATTCCATGCGTGAGTTTTCTGTGTTTATTTGCCTCG |
| | | | | | | 588 | |
| | | | | | 531 | 14022 | CGGCCTTTAGAGACAGATGCTGTCAGAGAAAAGACTTTGAACATTGACCA |
| | | | | | | 0803 | |
| | | | | | 532 | 12268 | CGGCGCAGACTCTCCCAGCTGGCACCAAAGCCTTCCGCTTCGCCGAGATC |
| | | | | | | 6453 | |
| | | | | | 533 | 30572 | CGGCCACCTAGACGCGCTCAGCGGCGCCCGCCCCACCCGGATCCCCGAAA |
| | | | | | | 739 | |
| | | | | | 534 | 75315 | CGCTCCCTCCCAGGGGACGGCTAGAGACTCACTGACTCATGCGTGTGTGG |
| | | | | | | 486 | |
| | | | | | 535 | 79083 | CGCGCTGACTCCCTCCGACCTGGGCTCCGATGGAGCAGAAATAGCGGGAC |
| | | | | | | 445 | |
| | | | | | 536 | 87908 | CGCTCCCCAAGTCTCTCTCGTGCGCAGAGCCCAGGCTGCGCTTCCCTGGT |
| | | | | | | 631 | |
| | | | | | 537 | 39755 | GGAGCCCCTGGCAGCCGCGGGGAGGGACGTGGCGGCCTGCGTGAGTGACG |
| | | | | | | 901 | |
| | | | | | 538 | 38036 | GGAGCCGTGGAGGCGGTGCCTTGGCTGGGGCAGTCTTGGGATGGCCATCG |
| | | | | | | 034 | |
| | | | | | 539 | 10005 | GAAAATCGAATTGAGTGCGGGCTCATTCGGCAGCGGCCGGCAGAGAAGCG |
| | | | | | | 0812 | |
| | | | | | 540 | 56304 | TCTCTCTGCCCTTGCGCTACTTTTTTTCTCCAAGGAAGGTTGTGCCTGCG |
| | | | | | | 28 | |
| | | | | | 541 | 22315 | CGATCACGTGAATAGGAGTGTGGCCGCTAAATCCGAATGGCTTCAGCATC |
| | | | | | | 4140 | |
| | | | | | 542 | 13421 | ACCCAGGGCAGGGTGGTCTTGAGTCTGGAGAAGGCCTTGCTCAGCACTCG |
| | | | | | | 0812 | |
| | | | | | 543 | 51184 | TGGAGAGTGCTGCTGGCCGCCAGTTGTTCCGGAGACGGTGCAAACGGACG |
| | | | | | | 379 | |
| | | | | | 544 | 52536 | GTTGCAGGAGATGAGCTCAGCGCAAAGGGAACCCCGCAGCGGCGAGTGCG |
| | | | | | | 175 | |
| | | | | | 545 | 39737 | CGCCCCCTCTGGCAGCACGGAAACCTCCACGCCATTGGCTGCCGAAAGCA |
| | | | | | | 768 | |
| | | | | | 546 | 29229 | CGCCTAGAATCAAGGCCTCTGGCTCGATTGCGTTCGGCAAGCGTCTGCAA |
| | | | | | | 249 | |
| | | | | | 547 | 72529 | CGGGCGCTTTCATGTCACTGCGGGTTTAGAGCTGATTCAAACTCATTGCG |
| | | | | | | 902 | |
| | | | | | 548 | 65027 | CGCGGTTACTGCAAGGATTCTGGCGAATGAGGTCTAAATAGAATGAGAAG |
| | | | | | | 63 | |
| | | | | | 549 | 14712 | CGCAGGCTCCAGCGCCCGCCAGGCAGATTCTGCCGGCCCGGCTCGCACTT |
| | | | | | | 7662 | |
| | | | | | 550 | 10274 | CAGGAAGCCAAGTTGGTAATCCCTTTCTTCTGGTCGCCTTGCTCACCGCG |
| | | | | | | 315 | |
| | | | | | 551 | 86331 | AACTCAACATGGCCTAAATGCATATCAGAAACTCAACATGGCCGGGCACG |
| | | | | | | 859 | |
| | | | | | 552 | 51522 | CGCCCCAGCTACCCTGGCTGCAGCCACGCCGCGCCCGAGGTTTCCCCCTC |
| | | | | | | 641 | |
| | | | | | 553 | 17699 | CGGGCCGCTCTGGTCACGCCCCTGACCCGCCCCTCAAGACCTGGCCTTAG |
| | | | | | | 746 | |
| | | | | | 554 | 38069 | GTAGAGCGGGAAAGCCCGAGGATCCTTTCAGCAGCACAGAGCAGAGACCG |
| | | | | | | 206 | |
| | | | | | 555 | 15525 | CGGCGAATCCCCGCTTTAGGAATTGCACCTTGGCCCTTTGTCGTCAGTCG |
| | | | | | | 9845 | |
| | | | | | 556 | 96632 | CGCGGGCAGCGAGCTTCAGGTCTTCTGATACATTGAAGGAAAGAAAGAAA |
| | | | | | | 841 | |
| | | | | | 557 | 47074 | CGCCGCGGGCACTTCTCCTGGGCTCTCCCCGAACTCTCCCGCGACCTCTG |
| | | | | | | 883 | |
| | | | | | 558 | 57618 | AAGGCGGCGCGCGCCAAAAAGATCTTCGGCTGGGGGGACTTCTACTTTCG |
| | | | | | | 943 | |
| | | | | | 559 | 17216 | GTGATATCAAGTGTCAGGGACCACCGGAGGCACTCAATAAATAGAAGACG |
| | | | | | | 7445 | |
| | | | | | 560 | 10089 | GAAAGAATAAGGCTCAAATGCAGGCCTCTGCAGCCTCCCTGTCATTCGCG |
| | | | | | | 4513 | |
| | | | | | 561 | 19789 | AGTGGTCGCAGTGCCGGGTGGAGTGAGAGCTCCGCTGTCTGCTGAGGGCG |
| | | | | | | 8229 | |
| | | | | | 562 | 18145 | GAGGAATGGAGCTTCGCAGAGGTTGCATTTAGATTCAACAGTTCACAGCG |
| | | | | | | 2707 | |
| | | | | | 563 | 49813 | GAATGGCAGCCCTGGCAGGGTCTGCGGCCGGCTGCGGAGGTGGGGACGCG |
| | | | | | | 033 | |
| | | | | | 564 | 72761 | CGGAGGGAAGGGTAAAAAATCTTGCTGCAATCCTTAGATAAATAAACCAG |
| | | | | | | 320 | |
| | | | | | 565 | 67766 | CGAGCCCTCCTGGCATCTGCACAGGCGGCCCCGCCCATCCCCGCTAGGAG |
| | | | | | | 4 | |
| | | | | | 566 | 62360 | CGGCCTGTTGAAAGTGCTGGAGCGTCTTTGAGCAGTTTGTTGTGATACGC |
| | | | | | | 464 | |
| | | | | | 567 | 78084 | CGCCAAAAAAGAATAAGTGCCTCCCAAACAACTGAATATAGGAGCTGTGT |
| | | | | | | 695 | |
| | | | | | 568 | 95239 | GCCTCCGCGCGGCTGCAGATTGAAGGGGCAACCCGATGGGGGAGGTCCCG |
| | | | | | | 381 | |
| | | | | | 569 | 17702 | GCGGCGGGGAGGCCGCCTTGCCGGGGTGCATGAGGCTGCGGAGTACTCCG |
| | | | | | | 4947 | |
| | | | | | 570 | 54423 | CGGCGACTAGTCGGTGCATGCTTTCTAGTACCTCCGCACGTGGTCCCCAG |
| | | | | | | 845 | |
| | | | | | 571 | 26663 | ACTTTAACCTGCAGCCGAAATGGAGTCTATAGGCCCCTTAAAAAAGCGCG |
| | | | | | | 593 | |
| | | | | | 572 | 97685 | CGGTCCCGATGACCAGGCCTCTTTAGGGTCCTGGCTCAGGCTGCCTTTGT |
| | | | | | | 274 | |
| | | | | | 573 | 30218 | GAACCCCAGGATAGAAGTTTTGTAGCCTATTCAGGTTGGAATATATGCCG |
| | | | | | | 600 | |
| | | | | | 574 | 14627 | CGGCTTCCATCTGAGCCCTATAGTCTCCAAACAAGGAGTGCATCAGCTGC |
| | | | | | | 8795 | |
| | | | | | 575 | 15032 | CGCAGTCACTGTGGTACAGTCCACATTCCGGCTCAGCGCCTGGCGAGTCC |
| | | | | | | 6174 | |
| | | | | | 576 | 74891 | GCCTGACTGGCTTCGGTTACTTCGCACAAAAAGCCCTTGCCGAGCAAACG |
| | | | | | | 207 | |
| | | | | | 577 | 14942 | CGCGTCTTGACATAAATCAAGGACTAAGAGAGACTGAAGAAGAGCGTTAG |
| | | | | | | 815 | |
| | | | | | 578 | 41875 | CGCCTTTCCGCAACCTACTGGCTCCGTAGATACAGCCATTCAGCCGGTTA |
| | | | | | | 470 | |
| | | | | | 579 | 89899 | TCCAAATTCCTAGACTCTATGTTACACTGCCTTTTAATAATAGTTCAACG |
| | | | | | | 729 | |
| | | | | | 580 | 66103 | CGCTTCAGGAAGAGAATCCAGTCCCAAGGCAAGGCACGCACATCCCCAGT |
| | | | | | | 889 | |
| | | | | | 581 | 10887 | CGTCATTTAAACGTTCCTTATTTGCGAAATTCCAAACAGATAGGCGTTCG |
| | | | | | | 047 | |
| | | | | | 582 | 21741 | CGCTGTCTGGGTCAACATTTCAGGGAGGAGTCTGCCACCAGTGTAATGTA |
| | | | | | | 492 | |
| | | | | | 583 | 48644 | CGAGACTGAGAGTGTCACAGGGATTGGACTTGACCTCAGACATCTTGACG |
| | | | | | | 408 | |
| | | | | | 584 | 19692 | CCGCGAATGGGGCTCGTGGAGCCTGCAAAGGAGTCTGCCTTTGGTATGCG |
| | | | | | | 172 | |
| | | | | | 585 | 16581 | CGCTGCCCTGCCCCGATCTCACAGAGTTATGACCGCCTCGTTGCAGTCCG |
| | | | | | | 2146 | |
| | | | | | 586 | 12030 | ACAAATGGAATATTAACATGCTCTTAAAGATATCAACAGTTGGTCTCTCG |
| | | | | | | 2922 | |
| | | | | | 587 | 10089 | CGCGCTCCCAGGTGAGTTCAGTGTGGCTGTGCAGTCACTTTCTTAAGACA |
| | | | | | | 6760 | |
| | | | | | 588 | 66327 | TAGGCTCTCCTGGGTTCTGTAACCCCGCCGTGGTACCCAGGTCCCATTCG |
| | | | | | | 000 | |
| | | | | | 589 | 29255 | CGCTATCCATCATCCATGTGACTGGGCGGAAGTGCAGGAGAGCAAGGGGG |
| | | | | | | 119 | |
| | | | | | 590 | 19788 | CGCTGGATACTGTTTTAAGAGCCGCGCCTTTCAAACCGAAGGGGCTCCTT |
| | | | | | | 2836 | |
| | | | | | 591 | 11645 | AATATTGATGCACATCTGTCCCCCGCTCAGGTGTAGAGGGGAGCTTTGCG |
| | | | | | | 1176 | |
| | | | | | 592 | 82763 | CGGCCAATCGCAGCGCGTCTCCGCCTGGCCAGGAGTAAGCCCGGCCAATA |
| | | | | | | 219 | |
| | | | | | 593 | 10848 | CGGGGCTGGAGGCTTCCAAGGCCTCACTCAGGCTCTTGGCAAGTTAGAAC |
| | | | | | | 9795 | |
| | | | | | 594 | 10510 | CGACCACCGTTCCTGCTCCTCTGGCGGGTGTCTTAAATGTGCTCTTGTAT |
| | | | | | | 2977 | |
| | | | | | 595 | 81471 | GCCGGAGGAGATGAAACCGTCTTGGCTGCAGTGTCGTAAAGTCACCAGCG |
| | | | | | | 938 | |
| | | | | | 596 | 35898 | ACTAAAGAAAATCACGTTATATTAGAAGCCTTACCCTGGTTTCACTTTCG |
| | | | | | | 975 | |
| | | | | | 597 | 26240 | ACTTTTGCAACTTTGCTACACACTGAAGCCCCTTATATATCAATTATGCG |
| | | | | | | 579 | |
| | | | | | 598 | 45018 | CGGCAGGTCACGATTTTTAAATGGACATGTCCTTAACTCACGTTGTGGGA |
| | | | | | | 949 | |
| | | | | | 599 | 63107 | CGAAGGCGCCCAGTTGCGAACCCTCCTACAGTCAGGCTGTCTGGAGCCAT |
| | | | | | | 73 | |
| | | | | | 600 | 14009 | CGCCTCCAGACAATTGGAGTGTACAGCCAGGGACACACAGCAGTGACAAG |
| | | | | | | 8392 | |
| | | | | | 601 | 15693 | CTCTCCTAACTGTAGAAATCCTTTGATCTCATAAAACATTTTCATTAACG |
| | | | | | | 3206 | |
| | | | | | 602 | 33170 | TCTGTCTGTGGGACTGTGGGTTCTCAGTGGAATTGTTGCCTTTCTTGTCG |
| | | | | | | 101 | |
| | | | | | 603 | 13141 | CGTGCCACTAACGAACTACAGAAGTATGAATTGACTCAAGGGGGCTGGCA |
| | | | | | | 0879 | |
| | | | | | 604 | 70992 | CCTTCCTAGACCAAGGGGTCATCTACGGCTCTGGACTTCGATAGGACTCG |
| | | | | | | 454 | |
| | | | | | 605 | 33700 | CGTGGCAACAGTCAGAAGGGTGGGAGCTTCTAGGCAATTTAACGCGGCAG |
| | | | | | | 801 | |
| | | | | | 606 | 63005 | CGCCCGCCCAGGAGAAGGCAGCTGCTACCTCTGCCTGCCTCCCGCTGCGC |
| | | | | | | 366 | |
| | | | | | 607 | 14572 | AGGCCTGCGCACGGGGAGGCTGTTGTGGCAGCGGAACAGAGACGTGCACG |
| | | | | | | 8507 | |
| | | | | | 608 | 72596 | CGGGACCACTCCAGCTTGCTACTATGGCCCATTCAAGAAATTGAGTAGGG |
| | | | | | | 351 | |
| | | | | | 609 | 84617 | CGCCACAGTCCAGCTGCCACAGCAGGACCAGCCAGTGCCCCACGGCACCC |
| | | | | | | 9 | |
| | | | | | 610 | 79083 | ACTATATAGATGAAGACATGAAACCTCTCTACACAGTGAACTCCCCCACG |
| | | | | | | 635 | |
| | | | | | 611 | 38892 | CCAAGATCGGTCTCACTGTAAGTCTGCTATCTGTATTTTCCCATTCTTCG |
| | | | | | | 887 | |
| | | | | | 612 | 13414 | CGCTCAAGTGCAGGCCTTTCTAAAGGAGCTGCTCAGCAGGCGGCAGCCCA |
| | | | | | | 3149 | |
| | | | | | 613 | 77122 | GAAAAGCAATCTTCAATGAATTTTAAACAATTATGGAGACTATTTCCTCG |
| | | | | | | 541 | |
| | | | | | 614 | 11184 | CGCCCTGGGGAAGCCCTGCCAGCCTGTTGCTCCGAAAACCGCATACCAGC |
| | | | | | | 7475 | |
| | | | | | 615 | 19749 | ACTCCAGAACTGAACCAGAGTTTTCTTGAGGTGGGATTTACTAGGAGTCG |
| | | | | | | 188 | |
| | | | | | 616 | 17519 | TGTTTCCTAATTTAAGAGGACCTTCGCCGGGCTAGAACGTCAGGGATTCG |
| | | | | | | 1045 | |
| | | | | | 617 | 26797 | CGTCGTCGTCGCCCAGCAGCAAGGGCTGCAAAATAGTAGAACTCGTGGTT |
| | | | | | | 576 | |
| | | | | | 618 | 30573 | GTAGGGTGCAGGAGGTTCCACGGACGCCTCGACGACCGGTAGTAGCTTCG |
| | | | | | | 013 | |
| | | | | | 619 | 11410 | CACAATGACATGTACAATGTCACTGAGAGGAGCTGGCGTTGTTGATTCCG |
| | | | | | | 9729 | |
| | | | | | 620 | 13655 | CGCAACCCTCCAAGCCCCAGGATCCAAAGTTTCCGTTTGAGAATCTGCAC |
| | | | | | | 4352 | |
| | | | | | 621 | 62584 | TTCCCGTTGACTTCGCTGCCTATGTGCAGCTAGGCTGGAAGCAGCAATCG |
| | | | | | | 600 | |
| | | | | | 622 | 22509 | TCGGACCGCGGCAGCAGGAGCAGGGCCGGGGTCATCCGTGTGAAGCAGCG |
| | | | | | | 01 | |
| | | | | | 623 | 30326 | AATACGTAGTAATTTATCCGTTCTCATGTTCTGTACTATCAGCACACACG |
| | | | | | | 592 | |
| | | | | | 624 | 36983 | CGCTTCAGATTGCGTTCTAGGTCTCTTCCCGCAGCTGCGACAATTCCCCC |
| | | | | | | 542 | |
| | | | | | 625 | 99439 | CGCCCACCGAGACCCCTGGGGTGGAGCTGTGCTAATAGAAACATACCCAC |
| | | | | | | 948 | |
| | | | | | 626 | 74826 | CGGTAAAGAAGAAGCTAAATCCTTCCTAGACAGAAGGATCCTGAAGCTGA |
| | | | | | | 447 | |
| | | | | | 627 | 84140 | AGGGCAGCCTGCGAGAAGCGCTGAGTCATGGCAGGAGATCATCCGAGCCG |
| | | | | | | 928 | |
| | | | | | 628 | 20001 | CGCCACTGATTAACTTAAAACAAACTCGGCTGCGTGTTTGTGCGCCGTTG |
| | | | | | | 1684 | |
| | | | | | 629 | 53359 | AAGCCCCTTTACGTCGGGGTGGGATTCCCCTTAAAGCCACTGCCAAGACG |
| | | | | | | 155 | |
| | | | | | 630 | 10475 | CGTCAATTATGGAGGCTTGGGATGTTAAAAGGCCAATATTTTTCAGTTGT |
| | | | | | | 2143 | |
| | | | | | 631 | 26250 | CGGCGCTTTCAACTGAAATTGTTGGTGGCTCTGAAAAGAGCCTTTGGTTT |
| | | | | | | 347 | |
| | | | | | 632 | 25129 | TGGGTGTCTGGGCCGGGGCCCCTCCAAGCCCACCTGGGAGAGCACTCCCG |
| | | | | | | 126 | |
| | | | | | 633 | 11410 | CGCTTCGGCCCCACAGTGCAGTTGTGCCTAAAACAGGTGAGAGTTTGAAC |
| | | | | | | 9420 | |
| | | | | | 634 | 10289 | TTTCTGGATTTTGCGCGGCCTGGCGCTGGGTGCCACAGCCGGTGAATGCG |
| | | | | | | 9285 | |
| | | | | | 635 | 13356 | CGCTACCCAGTTCCCCTGTGCAGGCCACTGCGGGCCCCCTGCCGGACTCC |
| | | | | | | 2035 | |
| | | | | | 636 | 15725 | CCCCAGTCGGGGTGCTGGAGCCCAAGCTGGAAGAGTTGGAGCACAGGACG |
| | | | | | | 652 | |
| | | | | | 637 | 66664 | ACCGAATTCCGGGTGGAAGGACCCAGCTGTATTGACCTTATATAAGCTCG |
| | | | | | | 798 | |
| | | | | | 638 | 89867 | CGGAGGCCACCCAGAGCTCACAGCCTCCTGCCAGCGCGCTCTCTGTTTCT |
| | | | | | | 819 | |
| | | | | | 639 | 24280 | GGATGCTGAGCTGCGCGGGATGCTGAGCTGCGGGGGGATGCTGAGCTGCG |
| | | | | | | 8479 | |
| | | | | | 640 | 17702 | CGGCAGATGCCGTGACAGCGACCATTGTTCCTGGTAGACACAGGCGACCA |
| | | | | | | 2646 | |
| | | | | | 641 | 49057 | AATGACCAGCTTTTAATGTGACTCCTTTAACCTGGTGTGCTTTAACAACG |
| | | | | | | 259 | |
| | | | | | 642 | 17267 | GCTGTTCACAATGCGCTCCGCCGGGCGGTGGAAACTTGGCTGCGGTAACG |
| | | | | | | 1526 | |
| | | | | | 643 | 82303 | GCCACGTCCGGGAGGATGAACGGAGCCCCGCTAAGGTCGGGAGGACTTCG |
| | | | | | | 50 | |
| | | | | | 644 | 18554 | CGGCTAAAATAAATACCCTTGTGAAATCTATAGTTATATTTAAGTGCACT |
| | | | | | | 0331 | |
| | | | | | 645 | 51147 | TCACGGAGGCCGGTAAATATGTAAACGTGGGTTTTTCGGTTTCCGTTTCG |
| | | | | | | 536 | |
| | | | | | 646 | 77983 | CGGTTCCCACCTTTTGCACTATGGACATTAGGAGTCAGATCATTCTTTGC |
| | | | | | | 27 | |
| | | | | | 647 | 63297 | CGTGCTTTGGATTCAGGTTATTTCAGTTTGATTCCAGAACATTTCAGAAA |
| | | | | | | 615 | |
| | | | | | 648 | 25941 | CGCTCTAGTCACAGCAGCCCACCATGCCTGCTCAGGCCTGGGAAGGGGCC |
| | | | | | | 33 | |
| | | | | | 649 | 17030 | CGCGTGGCCACGCAGCCCTAACCTGGCCCTCTTGGATTAGAGGGGAGCTG |
| | | | | | | 2890 | |
| | | | | | 650 | 66410 | AGCCGGCTTAGGACTGCGCCCGGGATGGGAGACCAGAGCGGCTGCTATCG |
| | | | | | | 998 | |
| | | | | | 651 | 13209 | GCCAGTAGTGATAAGCAAGCCTGGGAACCATGAGCTCTCCTTGGATGTCG |
| | | | | | | 0141 | |
| | | | | | 652 | 26598 | ATCTATCTTCTCACCTTGAGGTTCCAAAGATCATAACGGAAGGGGCTGCG |
| | | | | | | 303 | |
| | | | | | 653 | 11784 | GTCGGTTTCAGGACTCCCTCTTTTCAAGCTGCCAGAAACTTGTCTAGTCG |
| | | | | | | 7088 | |
| | | | | | 654 | 55095 | TCTGTTCTACCCACGGCGGCGGAAGCGACGGGGGAACTGAAGACGAGGCG |
| | | | | | | 177 | |
| | | | | | 655 | 12764 | CGGGTCTCCAGGTGTCAGAAAGCCAATCCTTACAAAGTTGAGTGATCCAA |
| | | | | | | 6592 | |
| | | | | | 656 | 10547 | GGCCGCCGGGCTCCGGATTTGCAGCCTGAGGCAGAGCTCGGGGGCTGTCG |
| | | | | | | 9248 | |
| | | | | | 657 | 71800 | CGGATCCCACCCCCACCTGGGCGCCAGACCCATCCAGCTGCTTGGAGCTC |
| | | | | | | 987 | |
| | | | | | 658 | 33258 | TGCTTGTCTCCCTTGTCTCTCCTTAGTAAGCGATACGAATCCCAGCTTCG |
| | | | | | | 515 | |
| | | | | | 659 | 53243 | GTAGGAAAGTTTTCAGGGCTCATTACTCGTGGAATATTGCGTTCCCCACG |
| | | | | | | 559 | |
| | | | | | 660 | 89744 | CGGGGGCCTGCGGATCAGCTCTGACTCTTCCTCGGACATCGAGTCTGACC |
| | | | | | | 701 | |
| | | | | | 661 | 76033 | AAAGGCGTTTTCTGCCCCCTCCGGGCCTCGGGAATATTTGTGGGCTGCCG |
| | | | | | | 097 | |
| | | | | | 662 | 11912 | TGCTTTATCCTGAGCTTGGTGGACCAGTTTCCTAGTAGGTGGGGTTGCCG |
| | | | | | | 1281 | |
| | | | | | 663 | 29396 | GCCTCCAGCTGGGTCTGTGCGCGCGGCAGAGATGCTGGGCGTGGGAACCG |
| | | | | | | 200 | |
| | | | | | 664 | 13529 | CGCCACTGCTGAGCGGTTGGAAGGGGAACACTGCAGGTACGCCCCAGTCA |
| | | | | | | 809 | |
| | | | | | 665 | 10531 | GTAGTAGATGTTTCCTGGCTCCTGGATGAGGGCCCTGGCCAGTCAGAACG |
| | | | | | | 9615 | |
| | | | | | 666 | 55401 | CACCACGCAAGGACTCGGGAAGCGGGCAAGTGGGCAAAACTCTGCTTCCG |
| | | | | | | 16 | |
| | | | | | 667 | 77719 | TGGGCATCTCGGCGTGGCCTGCGCCGGGGTCACGGGGAGGGCTGTCAGCG |
| | | | | | | 408 | |
| | | | | | 668 | 45429 | AAGGGCATGATAACTGCTTAAGTATCCACGTCTCTCATTTAAGACTTTCG |
| | | | | | | 449 | |
| | | | | | 669 | 81186 | CGCGCTGGATTTCAACCTCAGGAAACCCGAGAAGGGAAAATTTCCTTGAG |
| | | | | | | 795 | |
| | | | | | 670 | 89251 | CGGGCCCCTCACGCCCTACTTGCACCAGACCCCTCCCACCCGGGCCAGCG |
| | | | | | | 975 | |
| | | | | | 671 | 51385 | CGGGTCTACGCACCTAGGACCCGCTCGCCCTCCTCACGTGCCCACAGCTC |
| | | | | | | 916 | |
| | | | | | 672 | 18771 | GTTGCCTTGGTGACGGTGGTTGGGCTCGGGGTGTCCCGGTGTGCCCTGCG |
| | | | | | | 3986 | |
| | | | | | 673 | 49008 | CAGGAATGGAGTGTCCCCCAGCCCTGCAATTCCGTGGAGCGCTGAACACG |
| | | | | | | 322 | |
| | | | | | 674 | 50194 | CTGCCCGCGGCCCCTCCTGACTGCTAGTTGGAGCGGGGACAGCATGGGCG |
| | | | | | | 52 | |
| | | | | | 675 | 19788 | TTGCTACGAATTGGGCAAGTTCTGCGCCTTGAAACTGAACTAGTGGCTCG |
| | | | | | | 9693 | |
| | | | | | 676 | 63795 | GCTCCGCGGCGGGTGCCTTTCACACCTGGGTTTCCTTCGTAATTAATTCG |
| | | | | | | 711 | |
| | | | | | 677 | 19615 | TGGCTTCGTACCGGTGACTCGCGTCTTGGAGATTTTAACACTCCTCAACG |
| | | | | | | 480 | |
| | | | | | 678 | 17030 | CGCCCTGGGCCTCCGCGCCATATTCCTGGTCCCCCGCTCCCGGTGACTCC |
| | | | | | | 3540 | |
| | | | | | 679 | 11953 | TCGAGACGGTTCTATCCTTTTGCAAAGAAGCCGGAAAGAGCTGGGTCCCG |
| | | | | | | 2189 | |
| | | | | | 680 | 11884 | CGCCTGTGAGGGTCGCTCCTCAGCCGCCGCGCTCCCACTCCGCGTCCCCA |
| | | | | | | 2484 | |
| | | | | | 681 | 24197 | CGGCCATGTTCTAACCTGTACCACTGGGTGGAGGGAGAGCAAATCCTTTA |
| | | | | | | 863 | |
| | | | | | 682 | 80545 | CTGGTGTTGACCTAGAGGTGCCTGTTGTACTATGGTCACCGTCTGGACCG |
| | | | | | | 020 | |
| | | | | | 683 | 32115 | CGCCGCCAGCCAGCCAAAGCTCTCTCTACACTGGATCCCGGCCCCGTTTC |
| | | | | | | 19 | |
| | | | | | 684 | 73636 | CTGACAAACTTGTTCTGCGGGCTGCGGATGGGTGCGAGGGTGGAATCTCG |
| | | | | | | 184 | |
| | | | | | 685 | 31020 | GGAGAAGGCGGAGCGGGGGCGGCCTGGAGCGCTGATCGGGCGCCGAAAC |
| | | | | | | 806 | G |
| | | | | | 686 | 11372 | CTGAGGTCGGGTGTCAGGGCGCGTTGCGGCCTCGCGGGTGGGCAGAGGCG |
| | | | | | | 7753 | |
| | | | | | 687 | 17074 | CGCTCCCGCCGTCACCTCCACCTCCAAGCCCAGCTAGCCTGGGATCTGCG |
| | | | | | | 0673 | |
| | | | | | 688 | 27765 | CGGCCCCGCTCTTGAATCTATCTGAGGAAACGCAGCGAAGAAACAAGCAG |
| | | | | | | 232 | |
| | | | | | 689 | 24270 | CGGCGGCCTGCACCCGGAACTCACGCTGCCTCGGGTCCCAAAGGCTGAGA |
| | | | | | | 2553 | |
| | | | | | 690 | 69136 | GGAGGACAGGCGGCTCTGCGCTGCGGCTGTGGTGGGCTCCGTGCACACCG |
| | | | | | | 313 | |
| | | | | | 691 | 10184 | GAGCAGGTCTGACATCTTGCTGGGCTTTTTCAAAGAGAGCCACTGAAACG |
| | | | | | | 1416 | |
| | | | | | 692 | 32120 | CGGCTTCACACCAGTCGTTCTGGTCACTTAGGCGTTCGCGTGAGCGCTCA |
| | | | | | | 863 | |
| | | | | | 693 | 18101 | CGGCCAGACTGTTTTCAGAGTAGCTGATCCTTAAGTCACTGTATGCTTGC |
| | | | | | | 0312 | |
| | | | | | 694 | 50219 | CGCGTCATTACTGAGGTGGGGCAGAAGCAACAGTTGGTTACTCACCAGAT |
| | | | | | | 754 | |
| | | | | | 695 | 48690 | CGCTCGCGCTCTGCTCCGCGCCCGCCTTGACCCTGCCGCCCTCGCAGCCC |
| | | | | | | 457 | |
| | | | | | 696 | 14756 | CGGTGCCCATCTCGCACCCTTCCGCGTTGGCGGGCACGCACCACCACCAC |
| | | | | | | 1203 | |
| | | | | | 697 | 47695 | TGCAGGTTGGGGGCGGAAAGCTGTTTGCGATTCCCTGGACTGGTTGGTCG |
| | | | | | | 138 | |
| | | | | | 698 | 22904 | AGGATAAGTCTGTGGTGCTAGGACCCAGCTCCCAGAGTGCCAGACTGGCG |
| | | | | | | 6406 | |
| | | | | | 699 | 50817 | GCTCCAGGGAGACTCACCTTCCTTCTGAGAGGGCCGCACCCCTTGAGGCG |
| | | | | | | 213 | |
| | | | | | 700 | 22201 | CTCAGTATTCCTGCTGGCATTGTGACATGATTGAGCATGGCTCTTGTTCG |
| | | | | | | 921 | |
| | | | | | 701 | 10546 | GCGCAGCTCTCCGTGCCGGCGTCCCGGGATTTGTATTGCATGCATGTACG |
| | | | | | | 1368 | |
| | | | | | 702 | 17442 | AAAGTGGGGAAACCCAGAGGCGGGAGGCTCCCAGTGGCTGGGCAAGAGC |
| | | | | | | 2191 | G |
| | | | | | 703 | 11380 | TCAGGGGCGCTGGGGGCGCCACACCAGGTAAGGGGTCCAGCTTGGGAGCG |
| | | | | | | 0909 | |
| | | | | | 704 | 23287 | GCAGTCGGGAGACTGAGGGTTAGGGCGCGGCCGCGGGGTCTCCTGGGCCG |
| | | | | | | 352 | |
| | | | | | 705 | 47034 | CGCGTGTGCCCACACCTGTTTTCCCAGGCAGTCCCCTGAAAGGGGTGGTG |
| | | | | | | 267 | |
| | | | | | 706 | 35396 | CGGATCCATCCTCAATGTCCAGGCAGGACTCTCACATGAAGCCGGTGCTG |
| | | | | | | 475 | |
| | | | | | 707 | 76540 | GGGTCTGTGGCGGCGCAGCTTACACCGCGGTCCCCTTATTTGGATCTGCG |
| | | | | | | 438 | |
| | | | | | 708 | 34392 | CGCTGTGTGGAAACTCCGGTAAAACTCTTTGGGCAACAGTCTTATCACCA |
| | | | | | | 851 | |
| | | | | | 709 | 10094 | CGAAGTGTTCAGAATCCCCGTGCATTCCAGCGCGTTGACACTGAAGTCAC |
| | | | | | | 247 | |
| | | | | | 710 | 13908 | CGGGGCCCTGTCTGGGGTCTCTGGGAGAGTCACCCCCAACTTCCTGCCCC |
| | | | | | | 9315 | |
| | | | | | 711 | 12589 | CGCTCCCGCCATAGTCCCACCACCGGTGAGCGCCGCCCCCACTCGGCTGA |
| | | | | | | 9547 | |
| | | | | | 712 | 14937 | ACTTTAAATAAAAGGCTGATCTGACCTTCTTTTTGCAGAAGATGAATCCG |
| | | | | | | 5085 | |
| | | | | | 713 | 26250 | CTTAAACCAAAGGCTCTTTTCAGAGCCACCAACAATTTCAGTTGAAAGCG |
| | | | | | | 350 | |
| | | | | | 714 | 77870 | CGCCCTGGATAGCGACTTCAGAGAAGGAGCAGAACACGTATTCAGAGATA |
| | | | | | | 9 | |
| | | | | | 715 | 33297 | AATTGAGTGCGTGCACACTCAGTAAATGCGAAATGGTTTCATCACAACCG |
| | | | | | | 65 | |
| | | | | | 716 | 96889 | TTGATGGCGGCCGCGAACTTGGCTGCAGCGGCAGATCTCCGACCAAATCG |
| | | | | | | 169 | |
| | | | | | 717 | 18891 | CGCGGTCTAATCAGGCTACAGCCACCCATCAGCTCCGCCAGGGTTAGGGA |
| | | | | | | 6867 | |
| | | | | | 718 | 13959 | CGCGGCCCAGGAACAGCTAACGTGCCGCTTGCTGCTTGCCAGGTATGGAG |
| | | | | | | 3371 | |
| | | | | | 719 | 38724 | TTACTGAATTGTCTTGTTCAGGGACAAGTGGTGTCTCTCCCAGCTTCTCG |
| | | | | | | 255 | |
| | | | | | 720 | 64008 | CTTTGTCACCTATCCGGCAGAGGTACTCGGCCTTTGCCATCATTGCATCG |
| | | | | | | 041 | |
| | | | | | 721 | 65158 | CCAGATCATCCTATTACCATCACCATGCCTCATTAGAGTGGGGACTTTCG |
| | | | | | | 052 | |
| | | | | | 722 | 13421 | CGCCTGAAGGAAGCCCGGCGGACTCTGAAGAATGCTGCTGGCAACCAGCG |
| | | | | | | 7401 | |
| | | | | | 723 | 17330 | CGGCCGAAGCACCAGCGATTTCTACTTGGGCTTTTCCGCTCTACATGCAG |
| | | | | | | 3193 | |
| | | | | | 724 | 16491 | CGGTCCAAGTCTTTCAGGCTGTCTGAGCTCTTATTCGGGACACCCAGGCA |
| | | | | | | 1348 | |
| | | | | | 725 | 27173 | GACGTATAAGGCAGTCCAGGCCCTTCAGGCAAAGCCCTCTTAGTTAAGCG |
| | | | | | | 991 | |
| | | | | | 726 | 21323 | TCCTCAGGTCTGCCCAAGCAGCTTGTAGCTAGCACTGGGCCCCGTGCGCG |
| | | | | | | 15 | |
| | | | | | 727 | 38035 | CGCGGAAGCCCCTGCCTAGCGTCTCCCCATGGCCCTTGGTACATCCTCCC |
| | | | | | | 642 | |
| | | | | | 728 | 11949 | CGCTGACCCCAGCAGAGACCCTGGCTCTGAGCGAGTTCCCAGCTCTGTGA |
| | | | | | | 4494 | |
| | | | | | 729 | 19787 | GCCGTCCATCCATTCGATGGCCTCGCTGATGAGCATAGAAGATGAACCCG |
| | | | | | | 9766 | |
| | | | | | 730 | 44098 | CGCGGGCGGGCTGCCACATGAGGTCATTTCACAATCCCCCAGGATCAGGT |
| | | | | | | 287 | |
| | | | | | 731 | 35297 | AGCCAGCACCCCTGAGTGTCACTGAAGTTCCTGGATGGGGTGTGAGTGCG |
| | | | | | | 699 | |
| | | | | | 732 | 55443 | CGCCCCAGGCTCCGCCATGGGGAATGTGCCATCCGCGGTGAAGCACTGCC |
| | | | | | | 868 | |
| | | | | | 733 | 30526 | CGGGGCCGCAGGGGAGGCACTGCGGGGACGGGCGGGGTGAGCGGGAGCC |
| | | | | | | 92 | G |
| | | | | | 734 | 25907 | GGGTGACTTTCACGTGAAGCCCCTGCTGGGGGCCTTGCTGGGGTAAATCG |
| | | | | | | 218 | |
| | | | | | 735 | 15725 | AAAAGCGACAGCTCAGGTAAGGGCGCGCCAGGCACCCTCGAGAGTGCGCG |
| | | | | | | 477 | |
| | | | | | 736 | 62476 | CGCCGCGTCCACGATGGCTTCTTGCTCTGGAGCCCACCCTGCTTTGGCTC |
| | | | | | | 40 | |
| | | | | | 737 | 12924 | CGCAGACCTATGATGTGGAGAACTGGATCGCCAAAATAGAGACCTCTTGG |
| | | | | | | 3872 | |
| | | | | | 738 | 15306 | CGGCCCTGCCTCATAGCACGGATCAGCTGGGGACCCATGCGCTCTTCCTG |
| | | | | | | 1818 | |
| | | | | | 739 | 20669 | AGGACCCGGGAAGCAGGTGGGCGGACCACGCGGGGCTGAGAGGATCACC |
| | | | | | | 975 | G |
| | | | | | 740 | 84503 | TGGGATTTACTACAGGGGACCAAATGATACATTCAACCATATTATCAACG |
| | | | | | | 836 | |
| | | | | | 741 | 15090 | GCCTGCCATCGCCATGGTGACAGCTTCAGAGGCTGGAACAAGCTTAGGCG |
| | | | | | | 5602 | |
| | | | | | 742 | 28175 | GGCAGCTTTCCTGGGATATCACACCATATAGACTGAGAATTTCAGAAACG |
| | | | | | | 283 | |
| | | | | | 743 | 51147 | CGGCCCTTTGTGGCCCAGATCAAAGGACCCGAGCGCCACAAAGGCGGCCC |
| | | | | | | 902 | |
| | | | | | 744 | 15358 | TCTTTCTTTATTGGTAGCGGCCAAAAAGAGTTGATTGCTATTGGGATCCG |
| | | | | | | 4582 | |
| | | | | | 745 | 10886 | CGTCATTTAAACGTTCCTTATTTGCGAAATTCCAAACAGATAGGCGTTCG |
| | | | | | | 999 | |
| | | | | | 746 | 10299 | CGGGTGGAAGATCCCCCGCCCACCAGTATCCTGGATTTACCCAGGTCGAG |
| | | | | | | 6310 | |
| | | | | | 747 | 13290 | CAGTTGCCCTTTGTGTGCCTCAGTTTCCTCACATGGAACGTTAGATGACG |
| | | | | | | 8454 | |
| | | | | | 748 | 46387 | CGGCCCCTGCGGCTCTCTGTGGCCCACCCCCGCGAGCCCCATCCCGGAAC |
| | | | | | | 962 | |
| | | | | | 749 | 59287 | CCAAAGACTGTATGGTCTGTTATCTAACTGGTAGCCTTATTTGACCAACG |
| | | | | | | 786 | |
| | | | | | 750 | 61472 | AAGCAAGTGACAAGGACGCCCGAAGCACAGTGGACGGTCATGAAGGAGC |
| | | | | | | 147 | G |
| | | | | | 751 | 17513 | CGCTCACACGGTGTCTACCGGATCTGGCACGGTCAAGACACAGGAACAGC |
| | | | | | | 2842 | |
| | | | | | 752 | 10044 | CGCTCCTAGAAAAGAGCACGACGCACCCGATGCTCGGATTGGATGAAGTG |
| | | | | | | 2105 | |
| | | | | | 753 | 37395 | CGCTGCGGAGCGTTTTCGACTTTGCAAAGCTGTTGATGTTGGTGGGTCCA |
| | | | | | | 115 | |
| | | | | | 754 | 57261 | CTCCTCCGGTAGTCTCTCTGGACTCTCCAAGTGACTTGCTGGGGGCACCG |
| | | | | | | 601 | |
| | | | | | 755 | 50890 | CGCCTGGCACGACTACTGGGCCGCGTCTTGTCAGTCTATCAGCTTCTGAG |
| | | | | | | 678 | |
| | | | | | 756 | 13663 | CGCCATGCCCGAGCTACTCGCACTGCCTTGGTCCCATTCGGTTCCCCGGG |
| | | | | | | 3004 | |
| | | | | | 757 | 21689 | GAAATTTAGAGGCCAGGCCTCGCATGGGGATAATGGATGGGCACAGGACG |
| | | | | | | 759 | |
| | | | | | 758 | 47009 | CGCCTGGAAACCCAGCCGCGTGGGAGAGGGCGACCCCACCTCCAACCCCC |
| | | | | | | 707 | |
| | | | | | 759 | 19788 | CGGCACCAATAGCCCTGAAAATTAGGGAACAGCAGGGGCTGCCAAATATA |
| | | | | | | 8512 | |
| | | | | | 760 | 31819 | TGGGTGCCGTTCACCTTCCAGCCTAATCCGTGAGAAAGCGAGTGAAAGCG |
| | | | | | | 464 | |
| | | | | | 761 | 72275 | CGGAATGGTGCTTTCATTTACATATTAGCAATATAAAGTTATGATTGTGT |
| | | | | | | 551 | |
| | | | | | 762 | 11565 | CGGAAGCTGATGTAGGAGCAGCTGAATTCACCCCAGGGCCTGAAGGTCCG |
| | | | | | | 530 | |
| | | | | | 763 | 37123 | AACAAGGGCGAGGTTCCTGGAGCCCAAACTCAGGCCTCAGCCACCCTTCG |
| | | | | | | 949 | |
| | | | | | 764 | 66715 | CGCCCGCGAGACCAGCCTCGGCCACTGGCCTCCCAGGGCCAAGACCCACG |
| | | | | | | 7 | |
| | | | | | 765 | 10886 | TCTCGCTGCAGTTCATGGCTTTCGGGGAGTGACACGGCGGCTCCAGGACG |
| | | | | | | 8282 | |
| | | | | | 766 | 14376 | CGCCCTCGGAGGACGCATCCTAATCACCTCGGCAAAGAGCCGAGCCAGGC |
| | | | | | | 770 | |
| | | | | | 767 | 27958 | CGGCCCGGCCTGGGGACACACTCCCGCGCCCTCCAGGCCCCTGAGCTGTT |
| | | | | | | 421 | |
| | | | | | 768 | 46916 | CGCCCCCAGGCAGGCCGCCACCCAGCTCCAGGCTGACCTTGACGCGCCGC |
| | | | | | | 741 | |
| | | | | | 769 | 96959 | GCAAAGTCTCCAAAAAGGTTACAAGGGTTCCAGCGCCTCCCCAAGTCACG |
| | | | | | | 633 | |
| | | | | | 770 | 25090 | AGTCATTAACTGGCTGTCAGGTTCCTCAGATGATGGAGCTAAAAATAGCG |
| | | | | | | 198 | |
| | | | | | 771 | 11828 | CGGCTCCCTCAGCAGCACCATGGCTAGCTTTCATGCTTGCCTCAAGATTT |
| | | | | | | 4952 | |
| | | | | | 772 | 56258 | CGGGGTACCTCGCGCCCCACGCGAGTCAGAACCTAAAGTGCTCTCTGGGC |
| | | | | | | 973 | |
| | | | | | 773 | 52535 | CACATCTGGTTCTCGTTAACTTTTCTAAGGCAGCGGCCGCTGGAGCAGCG |
| | | | | | | 964 | |
| | | | | | 774 | 85482 | CGCTTTGGCTGGGTTAATTTTTGTCGATTGTTCCTCATCACACTCAGATA |
| | | | | | | 570 | |
| | | | | | 775 | 73641 | GCGCGTTGGAGGGGCGCCTCGGACCGGGAGGTATGGGGTCGGTGGCAGCG |
| | | | | | | 955 | |
| | | | | | 776 | 15429 | CGCCATCAGGAGTCAGGAGTCTGGATATGGACCAGGAAAGTTGTTCTACC |
| | | | | | | 955 | |
| | | | | | 777 | 55888 | TCACTGAGGTGGCGCTGAAACAGCTGACGGAGCGAAGCCTGACCCAGTCG |
| | | | | | | 548 | |
| | | | | | 778 | 66673 | CTAGGCGTCGAGGCGAGGGTTGAGCCTGTTTGGCTTCAGAGAACGATGCG |
| | | | | | | 316 | |
| | | | | | 779 | 84140 | CGGCCGGAGCCCTTGGCTCCATAGGGCCTCTGCCCACCATGGGCTTATGA |
| | | | | | | 993 | |
| | | | | | 780 | 32461 | CGTCTGTTCTGATGCCACCGCGAGATGGTCCCCGAGCTCCCCGAGAGTCC |
| | | | | | | 240 | |
| | | | | | 781 | 13664 | TCCTTGGACTGATAAAGTCAATCACTCACTCCTCGCACATAAACCGAGCG |
| | | | | | | 8249 | |
| | | | | | 782 | 13702 | AGTGGCACAGGGTGAAGGCAAACTCGATCGAAGAGCCCTCCGAGAAATCG |
| | | | | | | 8165 | |
| | | | | | 783 | 22316 | CGCAATTTGTCCATTCGAAATTGTCTCGAAGTTGTCTCGAAATTGTCCCG |
| | | | | | | 2881 | |
| | | | | | 784 | 10511 | CGGCCACCTACCACCCCTGTCCCCTGAAGATAAATGATCGCAGCTGCAAA |
| | | | | | | 4327 | |
| | | | | | 785 | 15666 | CCGGTCCTGATTGGGGGCGGGGGCGGGGAGCTCGGAGACCCAAAGGTTCG |
| | | | | | | 99 | |
| | | | | | 786 | 10715 | AGGGTGGGCTGGCTGGGAAGGATGGCAATGCCCATTGCTGACATCCGCCG |
| | | | | | | 612 | |
| | | | | | 787 | 25964 | CGGCACTCCCACTCCCACACCTGTGCAAACATCCGCCCGCTGGCCGGGAA |
| | | | | | | 16 | |
| | | | | | 788 | 13176 | CCATTTTTTCACTGGGCCTAATTCTTGACAATGGTTCTGATTCTCATTCG |
| | | | | | | 1763 | |
| | | | | | 789 | 90357 | CGCCCCACTTGCCTGGGCCGCCGGGCGCTGGAACCTGGACCCTGGACCCT |
| | | | | | | 992 | |
| | | | | | 790 | 28838 | TTTAAGTTTTTCCTTCATGATATGCCTGGAGCCCCAGGGGTGACAGCTCG |
| | | | | | | 631 | |
| | | | | | 791 | 13865 | CGGGGAAAGGTTTTTTAAAACTCGGTTTCATACTATTATTATTACTAAGG |
| | | | | | | 8243 | |
| | | | | | 792 | 79093 | GCTCCCGGGTGGGGCTTTCCTGGAGGAATCAGAGAGCACCAGGTAGCTCG |
| | | | | | | 913 | |
| | | | | | 793 | 97004 | CGGACTGGCAGTGTGCTGCTCTTCAAAGTAAGAGCTAGTGCTATAATTAT |
| | | | | | | 302 | |
| | | | | | 794 | 87138 | GAGGGAACGTGTGGGTCTGCCAGGGTCCTGCAGGGGGGATGAGACCCTCG |
| | | | | | | 510 | |
| | | | | | 795 | 27199 | TTGCTTTTGCGTTTTGCCTCTGGAACCATTCGACATCCGTGGCTGATGCG |
| | | | | | | 815 | |
| | | | | | 796 | 11540 | CGGAGTAGTTCTAAGAGGGTAACTGTTAGATTCTGTGTCAACCTCTGAGA |
| | | | | | | 407 | |
| | | | | | 797 | 29855 | GAGAATCTGAGTCCCGGTGGGTGCGTGGGGACTTTAGAACCGGGACCGCG |
| | | | | | | 465 | |
| | | | | | 798 | 34419 | CGGAGTCCGTTGTATGTCGACTTCCCAGGGCCGTAAGCGAACTTTGTTTT |
| | | | | | | 546 | |
| | | | | | 799 | 10456 | CGCCCCCATGCGGCCTGCAAGACACAGCCGGGCCTCCATCTCTGCAGGAC |
| | | | | | | 9519 | |
| | | | | | 800 | 12589 | CGCTGCGCTCCCGCCATAGTCCCACCACCGGTGAGCGCCGCCCCCACTCG |
| | | | | | | 9552 | |
| | | | | | 801 | 51410 | CGCCGAACTCATGTCTCCGAGGTGCCACCCGCCAGTGTCCCGCCCAAACC |
| | | | | | | 689 | |
| | | | | | 802 | 15950 | AGTTAAGCCAGTTGGGCTACAGATAGCTGCAGATTTCTCTCTCAACTCCG |
| | | | | | | 6621 | |
| | | | | | 803 | 11270 | CGGCTCGGGCAATCCTGCGCTAAAGCCCGCTCCCCCCGCCGCCAGCAACG |
| | | | | | | 9384 | |
| | | | | | 804 | 12853 | CGCCCGCCACCAGGAGTACTTCTCCCCGCCCGGCGATCCCTGCCGCCGCT |
| | | | | | | 1106 | |
| | | | | | 805 | 10128 | CGGGAACCATGACTCCACGGCCATCCGGCCCCAGGGAGGCGTTTTTCCAA |
| | | | | | | 2883 | |
| | | | | | 806 | 62364 | CGCCCCTGGCATTCAGGGAATGGTTTGCAAATCTCAATGCACCACAGACT |
| | | | | | | 315 | |
| | | | | | 807 | 10299 | CGTGCAGTTTATTTATTATGAGAAGCCTGACTCAGGCCTTTCCTGTGTCC |
| | | | | | | 6197 | |
| | | | | | 808 | 44187 | CGCCTCCCTGAGACCTGGGAGCACGTGCACTGGCTTTGAAGACTCCCGAA |
| | | | | | | 132 | |
| | | | | | 809 | 17702 | AGGAAGGGCGCAGGAGGCTTGGGACCTGGGCCCCATTCCTGGGGCAGACG |
| | | | | | | 5198 | |
| | | | | | 810 | 18385 | GGGCCAGTCAGGGTTCTCACTGATGTCTTGGGTGTGGCTCTGAGACTGCG |
| | | | | | | 672 | |
| | | | | | 811 | 75315 | AGAGAGGGCGAACGGGACGGGGACTTCCTGGAAGATTCCTACCACACACG |
| | | | | | | 527 | |
| | | | | | 812 | 49057 | ACCCTGCTAAGCAGTTGTCTCCAGGTCATGCACCTTCAGAATGGAAAGCG |
| | | | | | | 661 | |
| | | | | | 813 | 42992 | GGGATTCGGAGCTTCCCCAGCTGGGGTGAGCCTGGCGTTCCCGGGCGGCG |
| | | | | | | 774 | |
| | | | | | 814 | 11513 | TTTCAGGGCCCAATTAATTCGCCGCGGGCGGTGGGGCGGAGGCTGTGCCG |
| | | | | | | 6072 | |
| | | | | | 815 | 97148 | CGGTTCAGCATTAAAGTTCAAGTGGAGCGGAGGGGGCTGCATATTTTTAT |
| | | | | | | 0 | |
| | | | | | 816 | 17513 | GAACTGAAGAACCGCGCAGGAAAAGGCTGCCTGGACCAGCGGGACATTCG |
| | | | | | | 3151 | |
| | | | | | 817 | 62798 | TGAGAGCGAGGGACACGGGTGAGTCACGCGGCAGAAACTCACGGCAAGC |
| | | | | | | 365 | G |
| | | | | | 818 | 59189 | GGCGAGAGCAGGGATTGTCAGCAACTCGGGCTGCAGAAGCAGCAGCAACG |
| | | | | | | 843 | |
| | | | | | 819 | 11181 | CGGAGCGACCAAGGACAGGCTCTCGCTGGCACTCAGGTTGGGTTCTCTCT |
| | | | | | | 3690 | |
| | | | | | 820 | 16554 | CACTTCGGAGCAGCCTGTTGGGGGTCGTAGGCTCTGCAGGCTCAGAAGCG |
| | | | | | | 619 | |
| | | | | | 821 | 32455 | CGCCCAAGGCTCGGGAGAAGGGCTCTGCCCTTGTTGCCCGCCTTCACCCG |
| | | | | | | 025 | |
| | | | | | 822 | 11021 | CACAGCAACCAGCACCATGCCCATGACACTGGGGTACTGGAACATCCGCG |
| | | | | | | 0770 | |
| | | | | | 823 | 39453 | AGGGGTTGCTGGGGTGAAGCTCGGTGGGGCAGCCCTGCCGCCTGGCTCCG |
| | | | | | | 625 | |
| | | | | | 824 | 13159 | AGATGAGGCCAGCGGGGAGGCGGGTGCGTGAACTCTTGGGAGATTCTTCG |
| | | | | | | 3730 | |
| | | | | | 825 | 31940 | CGCCTCAGTGTAAGATGGAAACAACACTGATGTACAAGGCCTGTCATGAT |
| | | | | | | 855 | |
| | | | | | 826 | 86060 | TGCAAAGCATTATCTGTGGCTGAGCCCATCACAATTCAACAAAGATTACG |
| | | | | | | 076 | |
| | | | | | 827 | 39015 | CGGGCAAGTTTGGCTCAGTCCTTGTCGATGCTCCAGGCTCGGCTTCCCTG |
| | | | | | | 928 | |
| | | | | | 828 | 27196 | CGCCGCAGCACGTGACCGCCCACATGACCGCCTCCGCCAATCTCAGCAGT |
| | | | | | | 286 | |
| | | | | | 829 | 84417 | CGGCCCCCACATAGGGCCAGACTCACCCCCTGGGGACCCCCCTGTCAATC |
| | | | | | | 933 | |
| | | | | | 830 | 11153 | CGCGGCCTTGCTGCTGCGCCTCCAAAGATCGCGCACCAGTCTGGCCCACA |
| | | | | | | 2679 | |
| | | | | | 831 | 89120 | TTTAGAGACTCTCCTGGTGTTCACAGCTCATCGTTGTAAATGAGGGGTCG |
| | | | | | | 7 | |
| | | | | | 832 | 43800 | CGCTCCTCTACAAGGAGCACTCGAAATTTTGCCGTGGGCCGCCTGCGGAG |
| | | | | | | 133 | |
| | | | | | 833 | 32741 | CGATCCTGAATGAATGAATGGATGGACAAAGCGACGAAAGAACGCAGTGA |
| | | | | | | 518 | |
| | | | | | 834 | 16937 | CGCTTCATCTCAAAACCGTCTGGTGCTGAGGAAGTGGCCCAAGATGGACG |
| | | | | | | 6470 | |
| | | | | | 835 | 15727 | CTAGAAATGCGCGTGCAATCTACACATCTCATATGACCGGGACACAATCG |
| | | | | | | 127 | |
| | | | | | 836 | 57090 | TGAATGGCAGGAGCTGCACCAGGGGGCTGTGCGCGCTGCGCGGGTGTGCG |
| | | | | | | 015 | |
| | | | | | 837 | 10289 | GCAGGTCTATTCCGCCGCTTGGGCAAACAGGCGGGTTAGTGCACTCCACG |
| | | | | | | 4441 | |
| | | | | | 838 | 37390 | GGATGGACGGAGTAGGGGTTCCCGCTTCCATGTACGGAGACCCTCACGCG |
| | | | | | | 326 | |
| | | | | | 839 | 17073 | CGGACCAGCACTCCACTGTGGGTCCAAGGATGAGCTCCAAAGAGCCCAGT |
| | | | | | | 594 | |
| | | | | | 840 | 79083 | CCTGCCCCGGGGAATGCGAGGCTGCTTCCCTTCTGCGTAATCAGGGTACG |
| | | | | | | 753 | |
| | | | | | 841 | 55589 | AAGCTTTCTTTGGGTGAGGCAGGGCCGTCCTGCCCAAAGCCCCTCCTCCG |
| | | | | | | 803 | |
| | | | | | 842 | 21915 | CGGAGCCTTAACCCTTCAAATACCCCCAGGAGAGCCCCAGCCGGGGCCTC |
| | | | | | | 1444 | |
| | | | | | 843 | 24803 | TGCTCGCAGTGGCCTGGGCCAGCGGCAGGGTGAGCCGAGCCTCTGCTGCG |
| | | | | | | 679 | |
| | | | | | 844 | 11421 | CGGAGGGCCCCCGTCTCCAGAGAGAGCCAAAGAGTCAGGCTGACTGCTTT |
| | | | | | | 3914 | |
| | | | | | 845 | 22931 | ATGTAGTGTTAACGGAAGAGTTTAGGTCCTGCCTCACTGTCCGGGAAACG |
| | | | | | | 71 | |
| | | | | | 846 | 15825 | GCTAGCTCTTCTTCTCCCAGGTGGTGACAATGCAGACGGTAAGAACATCG |
| | | | | | | 9926 | |
| | | | | | 847 | 18771 | CAAGACGACGGCGTGCAGGCTGGTTGCCTTGGTGACGGTGGTTGGGCTCG |
| | | | | | | 3964 | |
| | | | | | 848 | 39466 | TCCCTTTCCCACTTTGTTCTGGGAGGCAGTCGAGGGGCTCCCCGTGATCG |
| | | | | | | 313 | |
| | | | | | 849 | 86383 | TGCTTCCAGGGCTTGGTTTCGCTTTGCCTGGCTCTTGGCTAGGCCTGGCG |
| | | | | | | 182 | |
| | | | | | 850 | 13907 | GAGGCAGCTGGGGCGGGGGTGGCCGCGGGAGCGTCTTGCCCGTGTGATCG |
| | | | | | | 6670 | |
| | | | | | 851 | 89676 | TCATGGCCACGAAGGCATCGTGGTAGTGCCGCTTGGAGTTGTGGGCCACG |
| | | | | | | 3 | |
| | | | | | 852 | 11139 | CATTCCACCCCCCTTGTTTCCGCATTCATCCTGAGTGGCTGGTGGGAACG |
| | | | | | | 7401 | |
| | | | | | 853 | 91957 | TCGGCTGTGATGGGGAGCCCGGAGCAGCCAGGCGGCGAGCAGTAGCGGCG |
| | | | | | | 992 | |
| | | | | | 854 | 51430 | GCTGGTACTGCTGGGGGTACCGAGTCTCCTCTGCTTTGGCAAAATCCACG |
| | | | | | | 288 | |
| | | | | | 855 | 11553 | CGCAAAGCTGCTCTCTCACTCCGTGGAGTTGGATCTCTACCCCCGCAGCC |
| | | | | | | 0590 | |
| | | | | | 856 | 12190 | GAGGGGCACACGCCGGAGCAGCTCCAGCCTGGGAGCATTGGGCGTGAACG |
| | | | | | | 3520 | |
| | | | | | 857 | 11708 | GACGGGGATAGCGAGCTGAGTGTGCGAGCGCCAGGGGTTCCAGCTGCACG |
| | | | | | | 6850 | |
| | | | | | 858 | 11515 | ACAAAACAGGCACTCTCTGGCCTAGGAGTGGAATCCATCCTCAATTTTCG |
| | | | | | | 2785 | |
| | | | | | 859 | 29293 | CGGGATCCTGCTGGGGGCTACAGCCCAAACATGTTACTTTTCCAACTGTT |
| | | | | | | 450 | |
| | | | | | 860 | 64008 | CGGCAGAGGTACTCGGCCTTTGCCATCATTGCATCGCGAATTTCGCTCTC |
| | | | | | | 007 | |
| | | | | | 861 | 12869 | CTTGAAGCCTGCTCGGAGCTTGGGATTGTATTTTCTTTGGCTTTCTGGCG |
| | | | | | | 4303 | |
| | | | | | 862 | 21251 | CACAGACGGCTGCTGCTCCACCCTGGGGGCCCTGCAGGGATCAGGTGCCG |
| | | | | | | 82 | |
| | | | | | 863 | 11696 | CGCCCACAGCCCAATTCCCCAGGCGCAGCGAGCGCCTGCAGCCCGAAAGC |
| | | | | | | 3259 | |
| | | | | | 864 | 28998 | CGCAGACTTGGGTCCCTTCCGGACGCAGCCCGCGTGCTCCCCGAGCTATT |
| | | | | | | 214 | |
| | | | | | 865 | 10381 | AGTTCACACCGTACGGGTCGGCTCAGAGATGAAGCCAAAACCTTGGGTCG |
| | | | | | | 3150 | |
| | | | | | 866 | 16097 | GGATGGCGGCGGGTGGGGAGTGCGCCTGCACGTGTGTGCGCGCGGGTGCG |
| | | | | | | 4837 | |
| | | | | | 867 | 10191 | ACCGGAATTACAGCATTTCTTGGTAGAGGAAACTATCCCTCAGCCAACCG |
| | | | | | | 5091 | |
| | | | | | 868 | 14020 | CTGAGGCACCCTGGAAAAGTTCCCTACTTCCAAAGTCTGCAGGAGCTTCG |
| | | | | | | 1065 | |
| | | | | | 869 | 27015 | CGGGGAACCCCTCCTCCGGGGCCCTGGCAAGATGCTGGCTCTGTTGCCCT |
| | | | | | | 808 | |
| | | | | | 870 | 65290 | CGGCCGACTGGGGTCCTGTCCTCTTGTCACCTTGTATCCCTCGCATGCTC |
| | | | | | | 484 | |
| | | | | | 871 | 66664 | CGGGCATGTCTCTGGAGGAAGAAATTCAAAATCACAATAATAAAACGTGC |
| | | | | | | 535 | |
| | | | | | 872 | 13588 | TGCAGGCACCCCCGCGTGTATGGGTGTGAGTGTGCACGTGTGCCTGTGCG |
| | | | | | | 174 | |
| | | | | | 873 | 15852 | CCTTCTCCACATGCTTTTTGGTGGTGGGGGGATGTGGCTTCCCAAGGCCG |
| | | | | | | 4649 | |
| | | | | | 874 | 97319 | TTCTGACAGGCTTTTCCTTCATCGGTGGCCTTTGACTCCAGCTGACTTCG |
| | | | | | | 911 | |
| | | | | | 875 | 85307 | TTTCATAACCTGGCGAGAATCACTGAGCGCTATGAGGCCACCGCCTGACG |
| | | | | | | 131 | |
| | | | | | 876 | 11531 | CGGAGCCCCCGATCCCTCCGGGTCTAGCGCAGGCCCCACGGGGCCCCTTC |
| | | | | | | 469 | |
| | | | | | 877 | 37959 | GGATCGCTTCAGAACTTCAGCAAACATTCCACGAGGACTTCTCGGTGCCG |
| | | | | | | 465 | |
| | | | | | 878 | 75315 | CTGTCTTATACCAGCCCTGTGCGGCCAGGCAGGAAGGCTTCCGTGGTGCG |
| | | | | | | 108 | |
| | | | | | 879 | 49297 | ATCGCTAACATGGAGAGGCAACAGGCTAGAAGAGTCCCTGTCTTGGAGCG |
| | | | | | | 846 | |
| | | | | | 880 | 43741 | CGCATTTGTTGCTCATGGCGCTCGCCCCGTTCCAGGCCTCCCTGGTGCCC |
| | | | | | | 530 | |
| | | | | | 881 | 32213 | CGCTGGATCCTCTCCACATCCAGGCCGCGTCCACTGGCTTTCCAACCTGC |
| | | | | | | 28 | |
| | | | | | 882 | 22106 | CGGCTTCTTGAGAGCAGGGCTGGACTCTGCTGCATGGTTGGAGAAGCTCT |
| | | | | | | 4388 | |
| | | | | | 883 | 41749 | CGCGGAGACTCACTACCCCGACATCTACACTCGGGAGGAGCTGGCCCTGA |
| | | | | | | 443 | |
| | | | | | 884 | 63 | TCCTGCCCAAGACTGTGACTTCTGACCACCTGTACTTTAAAACCAGAACG |
| | | | | | | 0437 | |
| | | | | | 885 | 10289 | CGAGGCCGATAGCTGGGATGGGGCTGAAGAGCCCTGGCTCTGTTTTACCG |
| | | | | | | 4342 | |
| | | | | | 886 | 13494 | CGGCCCCAGGATGGCCCGCCCGTCTGCCCTCCGCCTTGCCCCGCCCACCC |
| | | | | | | 1229 | |
| | | | | | 887 | 27154 | TGAGCTGCGTGTCTGCACTCTGAAATGGGCACATGAGCCCTGGAGATCCG |
| | | | | | | 264 | |
| | | | | | 888 | 12925 | TCTTACGGGGTAACCTGTCAAGAACTGTCAAATACAATTTTAGGGAGTCG |
| | | | | | | 3169 | |
| | | | | | 889 | 78935 | CCCTGCAGAAGCGTCCCGACGGCCACATCGTGAGTGTGAGGTGAGGAGCG |
| | | | | | | 289 | |
| | | | | | 890 | 21414 | CGGAGGAACGTTAGGAGACTAGGGAAAGACCTCTGCCATTGGACTTTAAG |
| | | | | | | 9735 | |
| | | | | | 891 | 11513 | GGCTCTCCCCTTGGGCGGAGTGGGGCGCGGTGGGGAACACAGCTTGGACG |
| | | | | | | 6279 | |
| | | | | | 892 | 10090 | CGACGCCTCAGCCCGGGGAAGACTTGCACAGTACACATTTAGCGAGGATG |
| | | | | | | 5458 | |
| | | | | | 893 | 12043 | CAAGCGGCGCCACCGGATTCCATTTGTAAACTTATTAGTGTGCGCTCGCG |
| | | | | | | 5056 | |
| | | | | | 894 | 19788 | CGCACTGGCCCAAGGGGCAGAGAACATCAGAAGGAACCTGGCACATAACT |
| | | | | | | 1741 | |
| | | | | | 895 | 73847 | CGCCTAGAAGGCTGCTGGGACCCCAGACCCAGAGCCCCGGTCACACTCCC |
| | | | | | | 318 | |
| | | | | | 896 | 10280 | CCCGCTCACTGGGGAGCAGCCAGGGCAGGCCTACCTGGCAAGGGCCAGCG |
| | | | | | | 3787 | |
| | | | | | 897 | 22873 | TCGTAGCCTTGGAAACGGCTAAACAGCAGCGGGACGCGGGTGCGCATGCG |
| | | | | | | 6324 | |
| | | | | | 898 | 13536 | CGCCATCAGCCTTCCTGGGCCTGAGCGGTCAGCTGTTTCGGAAAGTCCAG |
| | | | | | | 991 | |
| | | | | | 899 | 54349 | CTGCCTGCGGCTGGAGGCGGCAGGGATTCCGGAGTTGGGGGATCCTGACG |
| | | | | | | 571 | |
| | | | | | 900 | 10325 | TAAGTCTTTAAGTTATTAATACAACGACAACTATATAACTCCAGTTACCG |
| | | | | | | 0866 | |
| | | | | | 901 | 13970 | CGTGCTTAATGCTTGGCTGTCTGGTAAGGAAAATAAAGTGGTGCATTTTG |
| | | | | | | 4416 | |
| | | | | | 902 | 13112 | TGGGGTAAGTTGGTATACGGTTCAGCTAGCACACTGGCATTTTCTCTACG |
| | | | | | | 2594 | |
| | | | | | 903 | 32915 | CGCCTAAATCTTGGCCACCAGCGCAAACATATGTCACCCGGCCCTGGAGC |
| | | | | | | 497 | |
| | | | | | 904 | 31799 | CAACATAGCGAAAACCCATCTCTACAAAAAACAATAGAATTAGCTGGCCG |
| | | | | | | 444 | |
| | | | | | 905 | 28502 | CTGGCCAGTTGGCTGAGGGTGGCCGCTCAGAGTTTGAAGTACACATATCG |
| | | | | | | 630 | |
| | | | | | 906 | 27153 | ACTTGGGTATGTTTCTTATGAATATTACACGCGGAGCAGCGTCTGGTCCG |
| | | | | | | 655 | |
| | | | | | 907 | 19789 | CGCTTCCGGCCCCGCCCTCAGCAGACAGCGGAGCTCTCACTCCACCCGGC |
| | | | | | | 8217 | |
| | | | | | 908 | 70710 | CGGCGGTCACATTCTAAGTCTAGGAGTCGGCGACGATCCAAAAGCCCAAG |
| | | | | | | 389 | |
| | | | | | 909 | 31739 | TGGGGTGGGGCCTGCTCTCTAGGGGACTCTCAAGCTCTTGGAAGGTCACG |
| | | | | | | 521 | |
| | | | | | 910 | 11021 | GTCATGGGCATGGTGCTGGTTGCTGTGGATTCTGCAGACAGGCCTCAGCG |
| | | | | | | 0699 | |
| | | | | | 911 | 75602 | CGGCCAGTCTTGCCAGCACGCGAGCCCCTCCGCACTCACCAGTCCCTCTT |
| | | | | | | 412 | |
| | | | | | 912 | 48938 | GCACCCTGGATTCAGGCCTGGGTGGCTTGAATTGAGCCTCAGAAGAGCCG |
| | | | | | | 576 | |
| | | | | | 913 | 11271 | CGCCCCTCACTCGCTCTGGCAGCCAGAGGCCTTGTTCGAGGGCCTGGAGC |
| | | | | | | 7207 | |
| | | | | | 914 | 18399 | GGGACGAGGCAGAGGGGTCGGCCGCGGAGGGGCAGGCGGTGCCCGGCTC |
| | | | | | | 4073 | G |
| | | | | | 915 | 20001 | AGAATTTCTTTTAAATCCCTATGAAGTTAAATTCCAAAATGTCACCTTCG |
| | | | | | | 2526 | |
| | | | | | 916 | 74734 | GAGGTGGGATCTTGGGTGCCTGGTGATCCCAGTGTGTGGGGCTGGGAACG |
| | | | | | | 762 | |
| | | | | | 917 | 54422 | CGGGGATCGCAACCTCCAGCCCGTGGGCAACGCGTTCAGCCCAAGACCCC |
| | | | | | | 925 | |
| | | | | | 918 | 37394 | CGGTACTAGGAAGAGGGTTAACAGAACAGGACACTAAAAACAAAGTACTA |
| | | | | | | 589 | |
| | | | | | 919 | 95025 | GGGGAAGCTCGTGGCGCTGGTCCTGCTGGGGGTCGGCCTGTCCTTAGTCG |
| | | | | | | 611 | |
| | | | | | 920 | 50276 | CTGCGTTCTGGAGCTTATCAACACAGTGGATAATTAAAATCATAAACACG |
| | | | | | | 772 | |
| | | | | | 921 | 11828 | CGCAGCCCCAGACCCACAGGCCGAAGCAATAACCAGCCAGATAAAGTTTC |
| | | | | | | 5064 | |
| | | | | | 922 | 70563 | CGGCTCGCCCTTTGGGATCCACAGCTAAGAACTGGCCAGGAACGATGCGA |
| | | | | | | 092 | |
| | | | | | 923 | 57309 | GTAACTCCCCAAATGACTTCAGTTAACTGGGCCTTGAATCACAAGGATCG |
| | | | | | | 394 | |
| | | | | | 924 | 54321 | AAATCGCCTTTGGCGTGTGCAGCTCGAGCGTTACAACGATGCAAAGTGCG |
| | | | | | | 346 | |
| | | | | | 925 | 95239 | CGCAGAAGTCTATGAAAATGTGTTTGTTGAGGAGTCACTCGCCCCAGTAA |
| | | | | | | 751 | |
| | | | | | 926 | 13210 | CGCACCCCACCTCCGTCTCCACAGTGTAGCCCTCAGCCTGAGATGCTGAG |
| | | | | | | 7534 | |
| | | | | | 927 | 48145 | GCGGCAGAGGTGCCCTGGTTGGGCGGCTGGGCGCCTGGGGTTCGACGCCG |
| | | | | | | 108 | |
| | | | | | 928 | 15666 | CGGCCGCCCCGTCGAACCTTTGGGTCTCCGAGCTCCCCGCCCCCGCCCCC |
| | | | | | | 87 | |
| | | | | | 929 | 21667 | CGCACAATATAAAAACGCTGCAACAATGGAAGGAAGAAAGGTTTGGGTTG |
| | | | | | | 333 | |
| | | | | | 930 | 24147 | CGAGATGAAAGGATCCTGGCTAATTTCTTTGACCACTGTCTTGCAAATGT |
| | | | | | | 4238 | |
| | | | | | 931 | 27887 | CGAAAGTGAAGGAATCAAGTCTGGCCTATCATCTTCAGACGGCTGTACAC |
| | | | | | | 547 | |
| | | | | | 932 | 14852 | CTTTTGCGAAAGGGGCGTCTTTCTTCGTCGTGGAAGGGACCCTCACTCCG |
| | | | | | | 659 | |
| | | | | | 933 | 20145 | GCACGTGTGTGGGCGTCACTGGCGCCCTCTATGTGGGCGGAAATCATCCG |
| | | | | | | 0575 | |
| | | | | | 934 | 75602 | CGCGCTGCCCCGCACTCTGAGGGCCTTCATTAGCTCGCTCCCCGCGCCGA |
| | | | | | | 167 | |
| | | | | | 935 | 26088 | CTTCCCTGAGTGCTTGCCGAGAAGGCTGAGCAAACCCACAGCAGGATCCG |
| | | | | | | 202 | |
| | | | | | 936 | 77595 | ATTTCTTTTGTTACCTGTGATCCGGGCGGGGGGCTCCTTTCGGCCGCTCG |
| | | | | | | 321 | |
| | | | | | 937 | 51189 | GCAGTTCAATACCAGCAGCAAAACGCAGGTTTTTGGGGGAACTCCCGCCG |
| | | | | | | 941 | |
| | | | | | 938 | 41339 | CGCCCCGATGGCTGAGCCGCGGCAACACCGCGTCCTTAAGCCCCATCCTC |
| | | | | | | 785 | |
| | | | | | 939 | 11944 | GCCCGGGGCGCTGTGGCTTGATTTGCAGGTGGTTAGGTGGTTGTCGTCCG |
| | | | | | | 3387 | |
| | | | | | 940 | 19104 | CGCCCCCCGCAACTACCAAGCAGCTGCCGGCGCCGCAGCGGGAACAGACC |
| | | | | | | 5309 | |
| | | | | | 941 | 13421 | CGCCTTTGCGCTCAGCTCCCCGCTGGCCGCGAGCTCTCCAGGCACTCCTG |
| | | | | | | 7040 | |
| | | | | | 942 | 13788 | CGGGGTCGCCACTGCGCCCAGCTGCAGAAAGCACTCCGCACCCCCAGCAA |
| | | | | | | 68 | |
| | | | | | 943 | 19754 | GGCGGCCGCGGCGGCTGGACTCACGGGGTGGTGGTGGTGGTGCGGATGCG |
| | | | | | | 251 | |
| | | | | | 944 | | |
| | | | | | 945 | 46388 | CGCCCCCCCAGGCTGAGCCACGTTGAGTAAATGCATGTGCACGGTCAGGT |
| | | | | | | 390 | |
| | | | | | 946 | 24872 | AATTAATTTATAAATCTGCGTCCAATTAGGCAAGATGCTTTTAAATCTCG |
| | | | | | | 319 | |
| | | | | | 947 | 85482 | CGCGACGCTGATTTATGGTCCAGTCGCTCGCTTGAACCCCAACGCTTTGG |
| | | | | | | 612 | |
| | | | | | 948 | 52327 | CGGGATGGCCCTCCGCCTACACCGAAAAGAGAACAGACCACAGCCTTACT |
| | | | | | | 649 | |
| | | | | | 949 | 87975 | ATACCACCTTGTACACACATGAACACAGAAGTGCCGGGTAACCGCGATCG |
| | | | | | | 798 | |
| | | | | | 950 | 14708 | TATTCGCTGAACGTAGGGAAGTGTCCCAAAGAAACGCGGCTCCCAGTGCG |
| | | | | | | 7225 | |
| | | | | | 951 | 49315 | TGCTGGGGGCTGCGTGGCTGGCGGGCCCGGCCCGCGGGCAGAATGAGACG |
| | | | | | | 302 | |
| | | | | | 952 | 85414 | CGCCGAGAGGTGCTGTGGAAGGGTTCACGCTCACCGCATGGGGGACCTCG |
| | | | | | | 016 | |
| | | | | | 953 | 86383 | CGCGGTCCACCCATTGGCGGGAGAGGACTCACAGACCGCAGCTCCTCACC |
| | | | | | | 466 | |
| | | | | | 954 | 11389 | ACCCTAAAATTTTCTTCTGATTTCTGGAGACCGAGGAGTTTACCACTTCG |
| | | | | | | 7550 | |
| | | | | | 955 | 10289 | CGGCCTTTGCGCAGCCGGTCCCTACCCTGGAAGGAGAAAATCAATCCGCG |
| | | | | | | 4639 | |
| | | | | | 956 | 15032 | ACTAAAGGTGGATACCTCCTTCACATGGTGTTTGCTATAAGGGTCAAACG |
| | | | | | | 3558 | |
| | | | | | 957 | 11515 | CGCGCACATCCCCGGCTTCCCCGGGCTCCGCGCCTTCCCAAGAGCCCCGT |
| | | | | | | 2492 | |
| | | | | | 958 | 16237 | ACGTGGGCATGTCTTGGCCCCTCTTGGGCTATTAACTTGATTGACTCACG |
| | | | | | | 3963 | |
| | | | | | 959 | 11731 | GACCATGTGGCAGGAAGGCTGCCTGGTTCTGCACATCTGGCCCCTTCCCG |
| | | | | | | 4052 | |
| | | | | | 960 | 22094 | CGCTTCCTTACGTGGCTAATAAAGGGGTGCTTACAGAGCATGGAAAAACA |
| | | | | | | 563 | |
| | | | | | 961 | 10286 | CGGGGAAATGGAAGACAAAGGAAATGGGGGGACATAGCAACCCCTATTCT |
| | | | | | | 7826 | |
| | | | | | 962 | 30095 | GAGGGGCTGCGGCTCCGGGGCTCGGGTCGTTTTGAGTTCCTATCTTTGCG |
| | | | | | | 549 | |
| | | | | | 963 | 49309 | CGCTCTTAATGAGAACACAATTACCCAGATGGAGAGGGAGCCTCACCCCT |
| | | | | | | 124 | |
| | | | | | 964 | 12268 | GAGGATCTCGGCGAAGCGGAAGGCTTTGGTGCCAGCTGGGAGAGTCTGCG |
| | | | | | | 6456 | |
| | | | | | 965 | 59189 | CGGGCGTGCCAGCCTGGAGCTGTCCAACTTTTCAGCAGCTCCGGGATCAG |
| | | | | | | 934 | |
| | | | | | 966 | 29759 | CTCACTCCCATTGAGTGTCGGGTTTCTAGAGAAGCCAATCAATGTAGCCG |
| | | | | | | 947 | |
| | | | | | 967 | 53313 | CGCCCCAGGACCTGCACCCTCTACCGGCCACGGGACGTCCCTCCGCACCC |
| | | | | | | 529 | |
| | | | | | 968 | 10031 | CTGAGGGACCCCGGCCAGGCGCGGAGATGGGGGTGCACGGTGAGTACTCG |
| | | | | | | 8626 | |
| | | | | | 969 | 75680 | CGCTCACGCAGCACTTCTGGCAGTCCCTGAAGGACCACTACCTCAAGTCT |
| | | | | | | 535 | |
| | | | | | 970 | 31516 | CGGTGCAGTGGTGGGGGCTGCCCAGCATCTACATCGAGGCCTGGGTGTCG |
| | | | | | | 045 | |
| | | | | | 971 | 84808 | CCGAGAGCGGTTGGGTCAGCGGCTTCATGGTGCAGCCGAGTCTCAAAGCG |
| | | | | | | 65 | |
| | | | | | 972 | 73642 | CGCCTGGGGTCTTGCGTCCACTGGCCTGGAAAGCGAGGGCTATGCCCCCC |
| | | | | | | 885 | |
| | | | | | 973 | 27228 | CGGCATTGTGGATGAGGATGTCCAACCGTGGCTCAGAGCTCAGAAAGGCA |
| | | | | | | 511 | |
| | | | | | 974 | 78173 | CAGGTTTGTCCCCAGTTGATAGTTCCGTGAGTTCCTCAATTATTCCTCCG |
| | | | | | | 250 | |
| | | | | | 975 | 87980 | GCTAGGGTAGAAGCCCATGAAGGGGGTCCCGCGGCGCTAGCTTTTGTACG |
| | | | | | | 976 | |
| | | | | | 976 | 19243 | TCTGGATGATGTCTGTCCACCCACCGTCTCTGAGTGTTCTCCTCACCTCG |
| | | | | | | 756 | |
| | | | | | 977 | 14852 | ATTAATTTAGGGCTTTTTAGTCTCATCTGGAGAGTAAGCTCCAAGCCTCG |
| | | | | | | 905 | |
| | | | | | 978 | 53183 | CGGCAGCAGCGGTTTCAGCGGTGGCAGCGGCTTTGGCTCCATCTCTGGCG |
| | | | | | | 930 | |
| | | | | | 979 | 66360 | CACCTTGAATGATAGAACTTGAACAGCTCATTCATACAGAGTAGAACACG |
| | | | | | | 46 | |
| | | | | | 980 | 10823 | CGGTGCCCCTGACCTGCAGTCGTCACCTGCACTCCAGGCGGGCAACAACA |
| | | | | | | 7937 | |
| | | | | | 981 | 12510 | CGTCCCCGTTGCCATGGCGAGAGGCGTCCTCTCCTATGGAGAGTGACAGT |
| | | | | | | 2346 | |
| | | | | | 982 | 21682 | CTTCTTCCCATAGTTTCTTTGCAGCTAGCAGCTCCTCATTTCTCTTTTCG |
| | | | | | | 505 | |
| | | | | | 983 | 11944 | GGGAGTAGCTCCTAAGTAGCCTAGACGCTGATTCAGGCCCGGACTGTGCG |
| | | | | | | 2971 | |
| | | | | | 984 | 92940 | ATCTCTCCGGCTCTTACTTGAAAAAGCACTTGGAAGAAACTGTGTGTGCG |
| | | | | | | 163 | |
| | | | | | 985 | 14034 | AACATCCCTACAATAATTCTGTCTCAGTTTGCTGTGGTAATTTGATCTCG |
| | | | | | | 4290 | |
| | | | | | 986 | 15114 | TGGAAGAACTTTGGACAACATTTCCGCGGAGACCGGCGCGACCACCTGCG |
| | | | | | | 3125 | |
| | | | | | 987 | 37571 | CCCCGGGGTTTGCGAAGGTCCGGGGTAGGAGGGCTGCAGCCTGCGCGACG |
| | | | | | | 732 | |
| | | | | | 988 | 10173 | CGACCCTGGGGGTTCCTGCCATAAAACAGTGAGGGATTCTGGCCAGGCTG |
| | | | | | | 38 | |
| | | | | | 989 | 14711 | GGGCGCTTTAAACAAATGGCTGCCTCCCAGCGCTCTGAGTTAAGGGACCG |
| | | | | | | 0367 | |
| | | | | | 990 | 11840 | CGCCCTGAACTTGCAGGCAAGACCAGTGGCAATGATGGACTCTTCCCGCC |
| | | | | | | 7645 | |
| | | | | | 991 | 38965 | CAGTTCTTAAGGTCCAGCAACACAAAGCGAAAAGTTAGTGGAGGACTACG |
| | | | | | | 014 | |
| | | | | | 992 | 66461 | CGGAGTTTGGAGTTTGAACCCACCCGCACCCCTTTGAGCTAGGAAGTTTG |
| | | | | | | 802 | |
| | | | | | 993 | 13920 | TGCTGCGGCGATCCGGGTCGGGCTACGAGGGCAGCACCAGCTGGAAGGCG |
| | | | | | | 8852 | |
| | | | | | 994 | 11918 | CGGGCATGTCCAGGAACCAGTCTGCTTCCAGTGCCACCTGTAGTTGCAAC |
| | | | | | | 0915 | |
| | | | | | 995 | 33169 | CGCTCTCTACTTCAGATCTTGCTCAGTTTTGCTTCCGGTGGGCTCCTGGG |
| | | | | | | 606 | |
| | | | | | 996 | 65730 | CGGTTCCATGCCATGTTTTGGAATTTGTGTTTACGTCTGCCTTCATCCCC |
| | | | | | | 351 | |
| | | | | | 997 | 71955 | CGGCGCTCGTGTGAAGAAGTTAATTCAATTCGCTCTGATAACCGAGTTAT |
| | | | | | | 547 | |
| | | | | | 998 | 19788 | TTTTAAGCGAGGCTCCGGATGGCTGGGATGTCTTTAGGTTAGTAGTCCCG |
| | | | | | | 8880 | |
| | | | | | 999 | 31505 | CGAACCTTTCAAAACAGAACAGGCGTTTGGTGCCAAACAGGGCAGGGTGT |
| | | | | | | 024 | |
| | | | | | 1000 | 12725 | ACCAGGCATTGAGGCTCTGGTCCTACACACTGGGCATAGAGATGAACACG |
| | | | | | | 6676 | |
| | | | | | 1001 | 19743 | GGGATGGAGGGACGATTGGGGAGCGCCCGAGGCTGCTGTGTTGGCGCCCG |
| | | | | | | 029 | |
| | | | | | 1002 | 15402 | CAGGCACAGTGTGGGAAGAGTGCCATTCAATCCTCCGCGTAACCCTGGCG |
| | | | | | | 7129 | |
| | | | | | 1003 | 52832 | CGGCTTGCAGAGTCCAGTTAACAAGAGATTGAGCTCAGGAGTCCGAGACC |
| | | | | | | 660 | |
| | | | | | 1004 | 95026 | CGCGTCTCGGGAGCAATTCAGTGTCGCCTCACGTCTGGCCTTGCAGAAGT |
| | | | | | | 097 | |
| | | | | | 1005 | 42798 | GATCCTGCTGAATAAAGGGGGTTCCGAGCTGGGCCGAGATGGGGCATGCG |
| | | | | | | 747 | |
| | | | | | 1006 | 56642 | CGAACTTGGGAAGTGACTGAAAAACTTGCACTGATGAATTGGAGGGGTAA |
| | | | | | | 61 | |
| | | | | | 1007 | 17383 | TTATCATCATTGTATCGGCTTTACAACACTTAAAAACCAGACTTGAGGCG |
| | | | | | | 4077 | |
| | | | | | 1008 | 20145 | CGCCCACACACGTGCTGGTGTCCCGGGAAGAGTTCCTGGCAAAGAGCTCA |
| | | | | | | 0610 | |
| | | | | | 1009 | 15356 | CGCGTCTGCGATGGGAGCTCGATAAGGTCACCGTCAACAGCATCCACACG |
| | | | | | | 73 | |
| | | | | | 1010 | 14042 | TGTGCAAGCCGAGGTATTGGCCGCCATAACGCACACTGACTTTTGTACCG |
| | | | | | | 0502 | |
| | | | | | 1011 | 49297 | CGAGGATATCGCTAACATGGAGAGGCAACAGGCTAGAAGAGTCCCTGTCT |
| | | | | | | 901 | |
| | | | | | 1012 | 14635 | CGGCGCAGAGCCCCGCCAACTCGCCCGCTCCTCCAACCTTAAGTCGTTTC |
| | | | | | | 0131 | |
| | | | | | 1013 | 44327 | TATTTAACCACTGAGTCTAATTCGAAAAATCGGGACTGGGCCCCTAGGCG |
| | | | | | | 267 | |
| | | | | | 1014 | 65778 | CGCCAAACTTTCAATAGTTTGGAGTGTAGATGTGAGACTTCAGCTGCCCT |
| | | | | | | 481 | |
| | | | | | 1015 | 27196 | CGCCAATCTCAGCAGTCCTCACAGGTGGTCTCGCTCCGCAGGGCCCGCAG |
| | | | | | | 320 | |
| | | | | | 1016 | 16228 | CGCCGTCTAGTGGCGGAATTGTCCAGGGAAGGCAAGTTAGTTTTATCCTC |
| | | | | | | 3434 | |
| | | | | | 1017 | 13958 | TGCAGGTTTGCTCCAATCACAGCAGCAAGGATTGAAGCACCAACCAGCCG |
| | | | | | | 3634 | |
| | | | | | 1018 | 87417 | CGCCTTACAGCCTCCAAATACAGAGGCAGCAGCCAGAACCCCAAAGGGCG |
| | | | | | | 818 | |
| | | | | | 1019 | 45285 | ACACAGATGCGTGTAAGAGGCATCATGCTCTAAAACAGTGCAGAAATGCG |
| | | | | | | 821 | |
| | | | | | 1020 | 15793 | CGGAGTTGTCACTGTGCCACTTCCTGGACGCGGTGAGGAGGCTTCAGCTC |
| | | | | | | 2951 | |
| | | | | | 1021 | 36919 | CGGGATGCTACGGAGGGAGGTGTATACAGGTGTTAGACAAAGCCCCCTCC |
| | | | | | | 344 | |
| | | | | | 1022 | 19788 | CGGACAGGTACTATCTGCTGGCTGTGGACAAACAGTGGCATCTGAGATGC |
| | | | | | | 9163 | |
| | | | | | 1023 | 25891 | CGCAAAACGAACCCGATATTTGCAAAACTTAGGAAAAGACACACCGGCGT |
| | | | | | | 005 | |
| | | | | | 1024 | 11913 | CGGTCAGTGCCTGGCTGGGGACCCGGACCGGTGATTTGTTCAACTCTAGG |
| | | | | | | 3965 | |
| | | | | | 1025 | 20460 | CGCACCTTTACACCACGCCGCGCTGCCCCAGCCTGCGTGCTGCAAACGAC |
| | | | | | | 45 | |
| | | | | | 1026 | 16012 | ACGTGCTGCTAGAGTGGAACAAGTATGTCAGTTCATCCAAACATAAAACG |
| | | | | | | 2503 | |
| | | | | | 1027 | 21022 | AGACTCCCTACAGGTGGGCTACACTTAGGGGTTCAAATGCTGCATTACCG |
| | | | | | | 565 | |
| | | | | | 1028 | 81233 | CGGCCGTTGAGCTCATTTTCAAAGAAAAAGGAAGGAGGACACTGGAAAAT |
| | | | | | | 028 | |
| | | | | | 1029 | 11667 | TTTGGGAGAGCAGCTGCGAGGGGACAGAATGCTTCGCTGTGGTGGTTCCG |
| | | | | | | 735 | |
| | | | | | 1030 | 28457 | CAAAGCCTGCGGGCGGGGCTCCTTGCAGGTCCCCGAAAGAATGAGAACCG |
| | | | | | | 624 | |
| | | | | | 1031 | 48495 | CGCACAGCACCTCTCCAGCTCCATGCACGCCAGGGGCCTGGGACCCTGGG |
| | | | | | | 22 | |
| | | | | | 1032 | 75874 | CGGGTTTCTCCATATATGGTGTGCAGCTCACTTTGTGTTCTTAACCCTGT |
| | | | | | | 499 | |
| | | | | | 1033 | 19004 | CGCTTGAAAAAAGTTTTCGCAGACTGTGCTGGAGCTGGTGCTGAAAAAGG |
| | | | | | | 4504 | |
| | | | | | 1034 | 54261 | CGTCTTAAAACTTTGCACTTTAAGCTGGGCATAGTGATGCATGCCTGTAG |
| | | | | | | 853 | |
| | | | | | 1035 | 54421 | GTATGTGCGTGGAGAGTGGGGCGGCAGGCAACCCGAGTTCTTGAGCTCCG |
| | | | | | | 578 | |
| | | | | | 1036 | 53344 | CGCTGACTCCTCTTACTGGTTCTTGGGTAGCAGCCCCCAACTTGGTCTGA |
| | | | | | | 30 | |
| | | | | | 1037 | 10848 | TTTCCTGTCACCAGTCCCCGATCAAGCATGGTGTCAAGAAGATGCCTACG |
| | | | | | | 5152 | |
| | | | | | 1038 | 74179 | GCATTTTAGCCAGCAAGACCCGCTTGCATTGCAGTCGTTCACGGTGCTCG |
| | | | | | | 101 | |
| | | | | | 1039 | 11952 | GGTGCTCCTGCTGAACAGCCAGAGCTGGGGAGTAGACCCCGCTTCACACG |
| | | | | | | 9266 | |
| | | | | | 1040 | 50896 | CGCCAGCCCTCTCTGGGCTCGCCTCCCGGCTACCCCGGAGTCAGACTGCC |
| | | | | | | 251 | |
| | | | | | 1041 | 80657 | CGGCCCGCTAAAAGCATGGACTGTTTTGGCAGTCACTGTCAGACCTCCCG |
| | | | | | | 784 | |
| | | | | | 1042 | 91316 | CGGCGGTTCTTAGCTTTTTAGTCTCTGTCACGCTAATACATGCTGTTAGT |
| | | | | | | 253 | |
| | | | | | 1043 | 23791 | CGACTGAAAAGCCATAACATTGTTTCATGACTAGGATGTAGGCTAAAGGC |
| | | | | | | 473 | |
| | | | | | 1044 | 78176 | CGGAGAAAAAACAAAGCAAAATTTGTTCCCCACACGGTTTTTGGCTTTAG |
| | | | | | | 085 | |
| | | | | | 1045 | 12439 | AAGAATCAACATGGCGAGAAACTCTGTGGGTGACGTCCGTTTACAAACCG |
| | | | | | | 7892 | |
| | | | | | 1046 | 10044 | CCGAGCATCGGGTGCGTCGTGCTCTTTTCTAGGAGCGTGGGGTGCCTTCG |
| | | | | | | 2118 | |
| | | | | | 1047 | 63615 | GCAAGATGGCGGGAACGGCTACGGTCAAAGTGTCGCGATAGGAGCTTTCG |
| | | | | | | 383 | |
| | | | | | 1048 | 27513 | CTTCGATGACATTCAACTGACTTACTCTTCCGAAGGAACGGCTAAAACCG |
| | | | | | | 009 | |
| | | | | | 1049 | 54369 | GAGGCCGAGTCCAGGAGTGGAAGCCGCGCGAGTGGCGAAGGGAGCAGTC |
| | | | | | | 447 | G |
| | | | | | 1050 | 14675 | CGGCGACCACTCCGCCTCTGCCGTCCCACCTGACCGCAGAGGAGACCCCC |
| | | | | | | 5301 | |
| | | | | | 1051 | 10547 | TCAACTCAGGGCGCAGAACACTTGGGGTCTGAGTGTGCAAGGACCATGCG |
| | | | | | | 9383 | |
| | | | | | 1052 | 21525 | CACACAAAGACATGCGAAGAGGGGCTGAACGAGGCTCGCGCACAAAGAC |
| | | | | | | 6254 | G |
| | | | | | 1053 | 37004 | CGGGACACTGATCTGGGACGCTGGGACTCAGCATACACACACCTGGCGGT |
| | | | | | | 582 | |
| | | | | | 1054 | 20212 | CGGGTTACAACTAAACTGGAGAACATGGCCAAGGATGGGAACTCAGCCTG |
| | | | | | | 2309 | |
| | | | | | 1055 | 11271 | CGAGTGAGGGGCGCAGAGGCGGCAGAGAGCGGAGAGCCCCGGTGTCTCCG |
| | | | | | | 7244 | |
| | | | | | 1056 | 78935 | AGCCTGGGTGGTGAAGGCCTCCCTGCAGAAGCGTCCCGACGGCCACATCG |
| | | | | | | 269 | |
| | | | | | 1057 | 89745 | GTAATAATTATTCCTGACAGTGACCAGTCACTGAACATTCCAGGCTTACG |
| | | | | | | 356 | |
| | | | | | 1058 | 22437 | CGGTCCCCGTTTCCTCAACAGAGGTTAAAAAGCTCGTGCTCTGGCCGGGC |
| | | | | | | 2558 | |
| | | | | | 1059 | 87081 | CGACCCCCCAGTGGGGTTTCTTTCCGAGGGTCCCGCCCCTCGCAGCTGCT |
| | | | | | | 978 | |
| | | | | | 1060 | 39786 | CGCTGAGCTTTTTTGGTCAACTCTGGGATGTGTGCACCATCTAATCGGTC |
| | | | | | | 04 | |
| | | | | | 1061 | 15071 | CGTCCAGGGCCAGCCAGCAGCCCCGGGCTGCGCCCCCGCGCCCACCCCCA |
| | | | | | | 0730 | |
| | | | | | 1062 | 10072 | GAGTCCCTTCCAGCACTCTCCCCTTTGTTCCTTTTTCTGGGCAAGAACCG |
| | | | | | | 2408 | |
| | | | | | 1063 | 31826 | TGTTTTCCTATTAGTTGCTTCGCGGTCGAGTTCTAGGCGCATAATCATCG |
| | | | | | | 574 | |
| | | | | | 1064 | 14358 | CGCTCCCACCGCGGCACTCCGCCTCTGGGCCTTTGAGGCGCCTGGGGCCT |
| | | | | | | 0965 | |
| | | | | | 1065 | 60150 | GGACGTCGGCGCCAGGGCTTGGGGTGGGAACAGGTGCGGGGCTGCGTCCG |
| | | | | | | 3 | |
| | | | | | 1066 | 14635 | GAGGGGCACCACTCCGGGAGAGGCGGCGCTGGGCGTCTTGGGGGTGCGCG |
| | | | | | | 0585 | |
| | | | | | 1067 | 27526 | CCAGCGGTACTTGCTCCGTTAGGGGCGGTCAATTAAGTGGCTTCTTGTCG |
| | | | | | | 256 | |
| | | | | | 1068 | 68908 | TTCCTCTGCCTGAGGTGCTGACCTTGCCTCTTTCCCTGACAGTAAGAACG |
| | | | | | | 60 | |
| | | | | | 1069 | 15046 | CTGCCCTCTCATAGCAGGGAGTAATTGCCTTCAGGTAAAAGTTGATTCCG |
| | | | | | | 1119 | |
| | | | | | 1070 | 14402 | CGCAGCCTGGGATATCACACTGGGCCATCTCAGAGAGGAGAGGCCTCTGT |
| | | | | | | 157 | |
| | | | | | 1071 | 14489 | CGGCTCACTGGCCGCGTCGCCACCGCGTCCCTGAGTGCTCAGAACTTACC |
| | | | | | | 9592 | |
| | | | | | 1072 | 11694 | CGCCCGGGCAGCCTGGGAGGGGCCCTTTGACCTTGACCCCGCTCCTTGAA |
| | | | | | | 6163 | |
| | | | | | 1073 | 31696 | TCCAAATGGACCAATCACCGAGGAGCTGAGATCGTGGCGGACACGTTCCG |
| | | | | | | 229 | |
| | | | | | 1074 | 19573 | CGCCTCCGCCCCGCACCTCCTGACCCCCGCCCACACACGGCGTGGAGCGG |
| | | | | | | 65 | |
| | | | | | 1075 | 13110 | TGCAGGAGTTCCTCTGAGCTCCGTGGGGAACAGTGCTGGCCCCGAGTGCG |
| | | | | | | 1440 | |
| | | | | | 1076 | 86383 | AGTCTCAGGCGGGCATTCTGAGCTGCTTCTTGTCACTGGGGAAAACCACG |
| | | | | | | 585 | |
| | | | | | 1077 | 22094 | TGAAACTTGGAATATGATTCCATGATTCTCCGGAGTCGGGTTTTCAAACG |
| | | | | | | 479 | |
| | | | | | 1078 | 52627 | CGCCCGCCGCTAGGTCCATCCCGGCCCAGCCACCATGTCCATCCACTTCA |
| | | | | | | 047 | |
| | | | | | 1079 | 19548 | CGCCAGTAGTGTTGTGGATTGGGGAGGAAAGTGAACAGTAGAGAAACAAA |
| | | | | | | 878 | |
| | | | | | 1080 | 11403 | TTTTCATGGACCCCAGATAAAGACTTTGACAGCCAGATATCCAAGATCCG |
| | | | | | | 1854 | |
| | | | | | 1081 | 32223 | TCTCCCAGAACAACAGTTGTGATATGATCTGTTTTGGGGCCCTTCCTGCG |
| | | | | | | 336 | |
| | | | | | 1082 | 20145 | CGCCCTCTATGTGGGCGGAAATCATCCGGCGAAGATGAAGACGACCCCTT |
| | | | | | | 0601 | |
| | | | | | 1083 | 72756 | CAAGAGGAAGCGGCCCCGTGTGGCTGGGGGCGGCGGCGCAGGTGGTAGCG |
| | | | | | | 155 | |
| | | | | | 1084 | 15468 | CGGACGCATGACCTTCTGGAAGATGCCACCGTGCGGGGTGCCAGTCCCTC |
| | | | | | | 0808 | |
| | | | | | 1085 | 19749 | CGGCCACTACTGCTGTGCCATCATGTGGGGGTAGCCCATGGCCTACACCC |
| | | | | | | 961 | |
| | | | | | 1086 | 24119 | CGAGGCCATAGGAAGTGAGAGCGCCATCATACTAATGTTAAATAGACTGA |
| | | | | | | 8066 | |
| | | | | | 1087 | 46800 | CTGCAGTCCCTAAGGGTGGAGAGTGCGGTGGGGATGTGCCTCCAACTGCG |
| | | | | | | 479 | |
| | | | | | 1088 | 45914 | GGGCAGGCAGAGGCCTAGGGGGTGGGCTCTGAGGGCAGCCACTCCCACCG |
| | | | | | | 949 | |
| | | | | | 1089 | 10882 | GAAGCTGGGGACAATGGTTATGGACCCGGGCGGGGCCTTGTCCTTGGCCG |
| | | | | | | 238 | |
| | | | | | 1090 | 37571 | CGGGGCTGGGCGGCCCTGGGTGACAGACACAAGCTTCTGAACCCAAGCTA |
| | | | | | | 687 | |
| | | | | | 1091 | 66612 | CACATTTAGGCTGGAATGGGTGACAGCATGCGAGCTCGGGGGTGGGCTCG |
| | | | | | | 955 | |
| | | | | | 1092 | 35706 | CCTGCAACTTCAAGTCCCCAGGTGGCTCTGCCAGAGCGTTCACGATGACG |
| | | | | | | 038 | |
| | | | | | 1093 | 87800 | CGGCTGCCTTGCTGCAGCGAATCCGCACAATTAAAAGCTTTAATTAGTGG |
| | | | | | | 511 | |
| | | | | | 1094 | 87974 | CAGGTTACCGGCCAGAGTGGTGGCTCTTGGTGCAGGGCATGCTGCTCTCG |
| | | | | | | 464 | |
| | | | | | 1095 | 75315 | CGGGTTTGAATGAACCCTTGAGGTGAAGCTTAAAGACAGAGAGGATGCCC |
| | | | | | | 244 | |
| | | | | | 1096 | 49311 | CGCTCCGGCTCCCGACCTCCCCGCTGCCGAAGCAACTGGGTCAGCCTTTC |
| | | | | | | 782 | |
| | | | | | 1097 | 54391 | CGGGCTGCACAATGCCACGGCTGCTCCGCCAGCTCGCCCCTGCCCCGTCC |
| | | | | | | 325 | |
| | | | | | 1098 | 14572 | CGGCCCCCAGGCCTCTCACCGTGCACGTCTCTGTTCCGCTGCCACAACAG |
| | | | | | | 8526 | |
| | | | | | 1099 | 10909 | CGGTGGAGGCTTCTGTTCTCAATGAAATTCACTTGCGGCTTTTTAACTGC |
| | | | | | | 4032 | |
| | | | | | 1100 | 29139 | GGACGTCGGAAACATGCCCATGGGGTGGGCAGAGCTGCTCTGAGGTGCCG |
| | | | | | | 522 | |
| | | | | | 1101 | 28415 | GCATGCCCACGGCGACAGCGGCCACGTTAAGTGAGGGTCGTCTTGTTCCG |
| | | | | | | 023 | |
| | | | | | 1102 | 23171 | CGGCCCGCGACGGAGTTCCCAGGAAGAAGGCTGGCTGCAAAAACATTCCC |
| | | | | | | 2971 | |
| | | | | | 1103 | 17305 | CGGGCGCCTTGTAAGCGTCACATTTAGTTTTGCTGTGAAGCGAGTCAGGT |
| | | | | | | 79 | |
| | | | | | 1104 | 70482 | CGCCATGGGCTACTCAGTTTACAGCCACTGCAGTATCACAGCCAGGTCAG |
| | | | | | | 290 | |
| | | | | | 1105 | 25562 | GTAAGACCTAAAGTTCTTAGGAGATTGTCCAAAACAAAGAAACATGTCCG |
| | | | | | | 840 | |
| | | | | | 1106 | 57274 | CGCCCGAGGTCCCGCTCCACTGCCCGCGCCCCCCGCGCAGCCTTATATCT |
| | | | | | | 739 | |
| | | | | | 1107 | 38821 | TGGCTAACTGAACCTTATCGATAGGATGAGTCGCTGCGGCAGTCTGCTCG |
| | | | | | | 420 | |
| | | | | | 1108 | 42688 | CGCCCCGAGGCGGGGGCGCACTCACCACCAGCCAATGGGCGCATCTTCCA |
| | | | | | | 849 | |
| | | | | | 1109 | 23997 | TGCGGGACCCGCCAGAGTGTGCTTGGCTTCCGCGTGTCCGTGTGTCTGCG |
| | | | | | | 4554 | |
| | | | | | 1110 | 45914 | CGCCCGGCCTCCCTGCCTCTGCGCCTTCACCTGACCGCAAAGGCGGCCAG |
| | | | | | | 688 | |
| | | | | | 1111 | 35258 | GAGGTGGGGACGATGGCCGGAGTGACGCCCGCGGGAGCCCTGCTGGACCG |
| | | | | | | 594 | |
| | | | | | 1112 | 40657 | TCCAGAAAGGGTTAAACCCGGGCAAGCACCAGCTCCACGCAGTTAATCCG |
| | | | | | | 083 | |
| | | | | | 1113 | 13414 | CCCTCCCCAGGGATGTAGCTCTCAAGCAAACCTAGGCTGGGACGGATGCG |
| | | | | | | 7327 | |
| | | | | | 1114 | 12733 | CGGGGATCTATTGTGTCCCATTGAGGACTTAAATGAGCTGAATAATTGGC |
| | | | | | | 2027 | |
| | | | | | 1115 | 16757 | CCTACACCGCCGAGGACCGACTATTGTGAAGCCACTTTGGGAGCGGGTCG |
| | | | | | | 985 | |
| | | | | | 1116 | 86383 | GGGGTGAGGAGCTGCGGTCTGTGAGTCCTCTCCCGCCAATGGGTGGACCG |
| | | | | | | 464 | |
| | | | | | 1117 | 11633 | TACCAGCTGAGCCTGATTACACAGTCTGTGTGCCTGTCCCAGGACAGACG |
| | | | | | | 6029 | |
| | | | | | 1118 | 12331 | CGCCCCGCTGCGCCCACCTCCCCGGCTGCTCCCGGAGGGCTCACAAAGGC |
| | | | | | | 7487 | |
| | | | | | 1119 | 60075 | GCAGGTTGAAACAATCGAGTGGGCGCGCCAGGACTCCCCAGCTCCTGGCG |
| | | | | | | 23 | |
| | | | | | 1120 | 89744 | CGGACATCGAGTCTGACCTTGACCGAGACCCCAATAGTGCAACAGACTCG |
| | | | | | | 621 | |
| | | | | | 1121 | 32421 | GCTTGTCTGAGCGAGACCTAGACCGTGACGGTGCACGCCTACGAGAATCG |
| | | | | | | 808 | |
| | | | | | 1122 | 63795 | CGGTTTAATTAAGCCACAGCCCTAGCACGAGGACCCCGGCGACCCATCCG |
| | | | | | | 934 | |
| | | | | | 1123 | 11067 | CGGGAGGGGGCTTCTGGTCCCAGGCCCAAGACCGACCGCTTTGCAACTGA |
| | | | | | | 9107 | |
| | | | | | 1124 | 18141 | CGCCCCCTCGGACCTGGCGCCGCTGCGCCTGCTGGAGCCTCGCTTCCCCA |
| | | | | | | 3847 | |
| | | | | | 1125 | 51147 | CGCTGCAGATTAATTTTGTCATAAAATTGACTGGTGTGCTAAGGGACGTT |
| | | | | | | 461 | |
| | | | | | 1126 | 31516 | CGGCTGGTCCGGGCCCAGGCCCTCCTCCAGCGACACCCAGGCCTCGATGT |
| | | | | | | 015 | |
| | | | | | 1127 | 12331 | GCCTCTCTTTAAATATCAAGCTCACAAATATGTTTAATGAACTTGATACG |
| | | | | | | 3831 | |
| | | | | | 1128 | 27118 | CGCATTCTTTTGGTTTGAGGAATGTAGTATTGTCAGGTTGTTGCCTGCAC |
| | | | | | | 5 | |
| | | | | | 1129 | 10547 | CGCCGTCCTCCACCAGCACGTCGGCCACCTCCGAGAAGTCATCGTTGACC |
| | | | | | | 9058 | |
| | | | | | 1130 | 26897 | CGGCCAACACAGGAGGCACTGACCACGGGGCAGGCGCTATTTAAAAATCG |
| | | | | | | 6 | |
| | | | | | 1131 | 21251 | TGGCCTACTCACCTGGGGGCGAACTCCTTCTGGCACATGGGGCAGCTGCG |
| | | | | | | 14 | |
| | | | | | 1132 | 64343 | CGCAACACTCCCGCAGAAACACACAGCCCGGCAGCTTCTGAGGCAGCGCG |
| | | | | | | 315 | |
| | | | | | 1133 | 11860 | ATTTGAAATACAGACAGGATCCCAACCTGCACTTGCATGACCTTGTCTCG |
| | | | | | | 1891 | |
| | | | | | 1134 | 42075 | AAGGGAGCCCAACAGTCTTCATAAGCCAGACCTAGTCTCTAAGGTCACCG |
| | | | | | | 406 | |
| | | | | | 1135 | 20953 | GGGAGAGCATTTGCCCTTCTGTTTTTCTCCCTTTGTTACTGATTGGTTCG |
| | | | | | | 6324 | |
| | | | | | 1136 | 66122 | CGCCCAGACACCTCTCGAGGAAGGGAGATTGGACTTTCCCAGATTGGAGG |
| | | | | | | 804 | |
| | | | | | 1137 | 22624 | GCCACCAAATGGCGGCTCCGGCAGCCTGCTGCACTGATAAAGAGGACTCG |
| | | | | | | 374 | |
| | | | | | 1138 | 50892 | CGGCCTCACCCCAACGCAGTCCCTTTGGACGCCACCAGTTCCTGGGCGTG |
| | | | | | | 511 | |
| | | | | | 1139 | 10129 | CGGAATTTTTGTGCCCGAGCGCTCGGGATCCACGGCAAAACCAGGCACAT |
| | | | | | | 0699 | |
| | | | | | 1140 | 66041 | CGAAGACAGGTCTTGAATATAGACCCTGACTTTTTCACTGTTTGGTTTCT |
| | | | | | | 90 | |
| | | | | | 1141 | 87862 | ACAGATGTGCTTGCATCCGGGCCGTGCGTGCCTGCTTCACGGGTTTTCCG |
| | | | | | | 264 | |
| | | | | | 1142 | 32977 | CGCCCTCGCACTCACCCTTGGTGGCCCCTGCCTCCTGCGGGCTCAGGAGG |
| | | | | | | 217 | |
| | | | | | 1143 | 15718 | TGGCTCTCGGGTGTCAAAGGCTCTCCCAAATCTACATGGGGTGTTCCTCG |
| | | | | | | 5402 | |
| | | | | | 1144 | 85418 | GTGTCTCGTCCGAGGCCCTGGCTGTTCCTGAAACGGCCTGGCCAGGACCG |
| | | | | | | 371 | |
| | | | | | 1145 | 14079 | CGGCTGCGCAGACCTTGCCCAGCACACCAGATTGCCAGCTCCGAGACCCG |
| | | | | | | 7280 | |
| | | | | | 1146 | 58099 | CTCCTTCCCACTGTGTACCATGTACAGCATTGCTATTACAGGGAACTTCG |
| | | | | | | 11 | |
| | | | | | 1147 | 84815 | TAATTACCTCCTCTGCAAAGAGCTTTCCCTAGTATATTTTATTGATTGCG |
| | | | | | | 37 | |
| | | | | | 1148 | 12175 | CGCCCCGCCTCATTCTCGGGAAGTTTCAGGAACTTCCAGGGGAGTTGCAA |
| | | | | | | 8653 | |
| | | | | | 1149 | 16216 | GCTGGTGGGTTCTCAGTCAAGCAGGGCTGACAGCCGTCCCTCTTTGGACG |
| | | | | | | 215 | |
| | | | | | 1150 | 14966 | GGCGTTTTGGAGGTGGTTGCCATGGGGACCTGGATGCTGACGAAGGCTCG |
| | | | | | | 9368 | |
| | | | | | 1151 | 12617 | CGAAACCCTGGGAGACGAATCATACTGCAAATCATATCCCTGGGTAGCAG |
| | | | | | | 3246 | |
| | | | | | 1152 | 11004 | CGATTCAATTAGGCCGCCCCGCTCGGAAGCTTGTCTCCTCTGATATCCAG |
| | | | | | | 4639 | |
| | | | | | 1153 | 50884 | CGGCTTCCGCCCACCCATGGACTACGCCTTTAGCGATCTCATGCGTGACC |
| | | | | | | 480 | |
| | | | | | 1154 | 20000 | CGCGCCACGCCAGATCGAGATACTCTCATAGTGATTGTGAATCAATCGCC |
| | | | | | | 3800 | |
| | | | | | 1155 | 14023 | ATCTTATCCTTCTCTTGTTTTACAAATCGACGAATCAGCGGTCTAGAACG |
| | | | | | | 5586 | |
| | | | | | 1156 | 15400 | CGCCCGCAGCCACAGCCTCGGTCACCGCATCCATCAGGGCCCCGCGCCGC |
| | | | | | | 2064 | |
| | | | | | 1157 | 36919 | CGGCTTCACCTCTGGCACTCTCTGAAACGTCGTTGTCTATGGTTTCCCTA |
| | | | | | | 409 | |
| | | | | | 1158 | 15669 | CGGTAAAAGGCCCTGGTAGAACACTACATGACCCCTCTGATAAATCATGC |
| | | | | | | 5410 | |
| | | | | | 1159 | 32583 | CGTAGACGAACAAGGAAAAGTTTACTATGTGAGTAATAAGGCATTTGTAA |
| | | | | | | 696 | |
| | | | | | 1160 | 10675 | GGGGCTGGGTTTCTAAGGCAGGTTTCTAAGGTGTCTTCCTACAGACACCG |
| | | | | | | 0120 | |
| | | | | | 1161 | 37123 | CCTTCCCGCTAGGGCTTCTGGGAAGATCTACAATATTCATGAGGGAGGCG |
| | | | | | | 767 | |
| | | | | | 1162 | 14154 | ATGCTTGCTCAGGTTTATGCCCACATAAAACTTCTGAGCTGTTGTTTTCG |
| | | | | | | 788 | |
| | | | | | 1163 | 11138 | GAGGGCAGCCGGCAGGGGCTGGTACCAACTTGGCGGTTCTCGGAGGGGCG |
| | | | | | | 5450 | |
| | | | | | 1164 | 65498 | CGGGTAACTAGAGAGCTCTATCGAAGATGATCATTTGCTGGTCCTGCAGT |
| | | | | | | 537 | |
| | | | | | 1165 | 47696 | TTCCCGCTGCACTCCGGCTTCATCCATTTCTGGTCAGTTTCGTGGGTGCG |
| | | | | | | 701 | |
| | | | | | 1166 | 33592 | CGCCCTCCGCTCCCGCCATGGGGCCAACGTGGGGCTCCTAAGAGTTGCCG |
| | | | | | | 642 | |
| | | | | | 1167 | 49617 | CGGCAGGATAGTTTATGCCTGGGCTCCTGCGTTGGCATATGCGCCTTGAG |
| | | | | | | 042 | |
| | | | | | 1168 | 25486 | CCTTTCCTGGTGCCCCAGTCTTCTGCACTAAGCTGTGGTGAGCACATCCG |
| | | | | | | 849 | |
| | | | | | 1169 | 20871 | CGAGCCCACCAACCATGCCATCATTCAGACGCTGCTCAACTCCATGGCAC |
| | | | | | | 002 | |
| | | | | | 1170 | 50444 | GAGGTGCTGGGGAGCAGAAGCTGGGATCTTTCCGATTTCAGACTCCAGCG |
| | | | | | | 522 | |
| | | | | | 1171 | 11153 | GCCTGTCTCCCTTTACCCTGACGGTATTTTCAGAGGCACTAGGAGAAGCG |
| | | | | | | 2996 | |
| | | | | | 1172 | 11953 | TTGCAAAAGGATAGAACCGTCTCGACCAGGGCACTAGGACTGGAAGATCG |
| | | | | | | 2116 | |
| | | | | | 1173 | 11913 | CACGGCAGGAAGGTGGCGGTAAAGTCAGGGGCGAGCTCCTGGTCAGGGCG |
| | | | | | | 4323 | |
| | | | | | 1174 | 15731 | AATAGGGGGTGAGGACATTCAGATGCCCAGGGACCACTCGGCCCGTCACG |
| | | | | | | 4735 | |
| | | | | | 1175 | 10090 | TACTCTTAAAGTGCTAAAACAGTGTCCACTGCAGAGTAAACTTCCTTCCG |
| | | | | | | 6685 | |
| | | | | | 1176 | 18424 | CGCGCCCAGCTCCCTGTGAATACCCACTGATTGCCCCCGGAGTCTCCCCG |
| | | | | | | 3718 | |
| | | | | | 1177 | 81185 | TGAGATGTCCCCAGCAGGGGGCTTTGGGCGATTAACTTTTCGGAAAGTCG |
| | | | | | | 022 | |
| | | | | | 1178 | 41974 | GATGGAGTCACCCCCACCCTGGGAAATCGGGCACCTGACATGGACAGTCG |
| | | | | | | 087 | |
| | | | | | 1179 | 50889 | CGCAGAGAGGCGCACCGGACACGGTAATTAGGAGCAATTCACACGCTCTC |
| | | | | | | 442 | |
| | | | | | 1180 | 69452 | CGGCTGGTACCCTGAGCAATATGAACCCCCAAATCCACCAGAGATTCCCT |
| | | | | | | 398 | |
| | | | | | 1181 | 85403 | GGCCGGACTTGACCATTTTTAAAATGTTATGAATGACACATTTAGAGACG |
| | | | | | | 409 | |
| | | | | | 1182 | 11912 | TTGCTCTGGAGCTTCCCAGTTCCCAGGACTCTGGGGCTCTTCCCTGGACG |
| | | | | | | 5722 | |
| | | | | | 1183 | 27725 | GACGCAGCGCCTTTGTGTGGCGGCGGGTGTCGGGGGCGGGAAGAATCGCG |
| | | | | | | 188 | |
| | | | | | 1184 | 70150 | CGGCCCCGTTGATGCAACTGCATGCCATGGTTGCCAGAGAATCCTAGGTA |
| | | | | | | 722 | |
| | | | | | 1185 | 27647 | CGCCCTCCGAGTGTATGTCTATCCCAGAAGAAAACCTGTCTTAAAGTTCG |
| | | | | | | 713 | |
| | | | | | 1186 | 49468 | CGCGGCCGACCCCCTGTCCCCACCCGAGCAGGATTTCCTGATGTAAACAC |
| | | | | | | 988 | |
| | | | | | 1187 | 17700 | CGCATGTGCAGTGAAATGGCCTAGCCCGGGAATTCGATCGGCAAAGCACG |
| | | | | | | 4604 | |
| | | | | | 1188 | 13534 | CGGCGATGGCCAAATAATAAACTAACAGTAATATTATAGTAACAGCATCC |
| | | | | | | 2218 | |
| | | | | | 1189 | 10031 | GGACGCCTGGGTTGAGGCTCGCCGGGGCGTGAGCGGGGCTGCTGCAGACG |
| | | | | | | 8194 | |
| | | | | | 1190 | 76375 | AGCACACACCTTCGTCCGCATTTCAGGGACAAAAGTCGTCCAAGGAATCG |
| | | | | | | 33 | |
| | | | | | 1191 | 29387 | CGTGGCGGGCAGGGACATCACGGAGCTCCTCATGCAGCTGCTCCTGGCCA |
| | | | | | | 99 | |
| | | | | | 1192 | 85478 | GCCCATTTGTGCGGCGCTGCCTGGTGACTCAGCGTAGGCTGATGTTCACG |
| | | | | | | 296 | |
| | | | | | 1193 | 33700 | CCTAGAAGCTCCCACCCTTCTGACTGTTGCCACGTAGTTCTTAGTTTCCG |
| | | | | | | 817 | |
| | | | | | 1194 | 12024 | GTGCTGATCCCAGTGACAGAGCAGCATGAGTCACTGCCCGCCAGGGCTCG |
| | | | | | | 2330 | |
| | | | | | 1195 | 84140 | CGGCGCCAGCCATGGAGCCCGAAGCCCCCCGTCGCCGCCACACCCATCAG |
| | | | | | | 683 | |
| | | | | | 1196 | 11023 | CGGGCAGCCCTGAAGAGGAACGGTCCCTAGAGTCCGTCCCGCAGCTGCTT |
| | | | | | | 0633 | |
| | | | | | 1197 | 15350 | TGTGCATGCACGTGGGAGCCCAGCCCTGTAAGCACCATTCGTTATTTACG |
| | | | | | | 9096 | |
| | | | | | 1198 | 51891 | CGCTGTTTCAGACAACAACAAAGCCACAGGGAGGAGTTTAGCTGCAGTGA |
| | | | | | | 409 | |
| | | | | | 1199 | 10149 | TATTTGTGACGTAGGACACATGGTCTACTTCTTCTCAATATCACATCTCG |
| | | | | | | 1994 | |
| | | | | | 1200 | 29186 | CGAGGACCCTGCAGAACTCGAGCCTTTAAGCGCTCCTCTAGGCTCACATG |
| | | | | | | 153 | |
| | | | | | 1201 | 58055 | AACAGGGAGGCGAAGACAACTTTGTTTTTTCCCGGGAGAGCCCTCGCTCG |
| | | | | | | 876 | |
| | | | | | 1202 | 13616 | ATGAATTAGGCTGGAAACTGCCCCCATACCTCTAAGCTATGGGAAGACCG |
| | | | | | | 537 | |
| | | | | | 1203 | 11638 | AGTGGGTTAAAACCACAACCCTTGGGGAGCCCCAGAAGTGGCTCATCTCG |
| | | | | | | 0316 | |
| | | | | | 1204 | 62304 | ATATGCAGGTCTCACTTTAAAAGGTCTTGAAATGGAGTTCCTACCAGACG |
| | | | | | | 470 | |
| | | | | | 1205 | 12371 | CGAGAAAAGGAAATTTTAAAAGGCACAGGAAATGGTCCTACAGTAATCTG |
| | | | | | | 3553 | |
| | | | | | 1206 | 10374 | CGAGGCCCCAAGATCCATGTCGCCCGGAATCCCGGTGCCTAGGCCTTGTC |
| | | | | | | 0065 | |
| | | | | | 1207 | 17013 | CACAATCCTGGGTCGTCGGGGGACTGTACCGAGCGCCCTTGGAGTGCTCG |
| | | | | | | 6278 | |
| | | | | | 1208 | 56355 | TGTCAAAGGGCAGCAGGGCCCGGCCGTTGTCTTGGAAGCGCTGGTTGACG |
| | | | | | | 299 | |
| | | | | | 1209 | 15156 | CGGATATGGTTGGTGATTAAAAATCATTATTTTCTTTAGTCTGGGAGAAG |
| | | | | | | 1584 | |
| | | | | | 1210 | 12490 | GAACAACTAGGCGGGATGTACTTTTGAGCCCTGCCGGGTGTCTCCGATCG |
| | | | | | | 2844 | |
| | | | | | 1211 | 10506 | CGCGCACCCAATTCAGTCGCCCGCTCCCGTTCGGCTCCTCGAAGCCATGG |
| | | | | | | 6793 | |
| | | | | | 1212 | 12431 | AAGGGTAGAGTGGACTTCCCAGAGCTTTAGTACCTGAGACTGGGGACTCG |
| | | | | | | 1399 | |
| | | | | | 1213 | 80255 | CGGAGGGCTCCCGATGAACCGAGTCACGTTTTGTAACCACATTAACGGAC |
| | | | | | | 93 | |
| | | | | | 1214 | 47934 | CGCCCCCAACTCAAGGCAGGCTACACAAGATCTGTCTCCGTTGGCGTCCC |
| | | | | | | 802 | |
| | | | | | 1215 | 70984 | AGGAACATCTACCCGGCTAAGAGACGCCGCCAGAGTCCCTAAAGCTGGCG |
| | | | | | | 559 | |
| | | | | | 1216 | 80531 | GTCGTTTCCAATGACGGACATTAACCAGACTGTCAGATCCTGGGGAGTCG |
| | | | | | | 599 | |
| | | | | | 1217 | 17848 | CGGGGTAGCATAAGAACCTGCTCTAATTCCAAGGGAATTCAAGGAGTTAA |
| | | | | | | 4058 | |
| | | | | | 1218 | 19185 | CCAAGCTGATAACAGGATTAGTCGCACCGATAAGGAATGCTCTGACAACG |
| | | | | | | 165 | |
| | | | | | 1219 | 59189 | AGAGGTTGGTCCTTCTTCCTCCCTTTCTTCTTCCCTTCCTTTGGGCGCCG |
| | | | | | | 791 | |
| | | | | | 1220 | 22482 | ACAAGGGACATCTTTTTCACAGACTTGCCTTACACAGTTTCTTGAAGACG |
| | | | | | | 4500 | |
| | | | | | 1221 | 69762 | CGGAAATGAGGACAGGTGAAAGGAAGAGACCCAATCTTAATTAGAACGGT |
| | | | | | | 23 | |
| | | | | | 1222 | 24248 | TTGGCGTCGCGAGCGAGCGGTGGACTTGCCGGCTGGGTGCAAGCAGGGCG |
| | | | | | | 0504 | |
| | | | | | 1223 | 18067 | GGAAGGGCCGCCTCACTCACCTCGGGCGCCAGTAGGTGCAGGCTGGGTCG |
| | | | | | | 816 | |
| | | | | | 1224 | 33701 | CGGCTGAGGCAGGCTCCGCAGACCCCGAGCCAGAGTGGGATTTAACGGCG |
| | | | | | | 353 | |
| | | | | | 1225 | 58207 | CGACAAGGAGATCTGCATGATCAAGGTAGAGATCCAGGACATCAACGACA |
| | | | | | | 013 | |
| | | | | | 1226 | 62069 | CGGATATCGCGAAAGACACAGCTTAGCCCAGAGCCTATGCTAAGAACCCT |
| | | | | | | 806 | |
| | | | | | 1227 | 96294 | CGGGTAGTAGACATGCTTCTGGAAGGGCTGCGGGGGGCACAAAGAGAGCG |
| | | | | | | 131 | |
| | | | | | 1228 | 40719 | CGGAAACTTGAAGGCCTAAAAAATTAGCTGACCTTGTTAAAAATGTTGGC |
| | | | | | | 088 | |
| | | | | | 1229 | 16850 | GACCCCTGGGACAGGCAACTCTGGGGCCGTGTGACCTCCAGGACGGTTCG |
| | | | | | | 2341 | |
| | | | | | 1230 | 11004 | TGATTCCTGCTCCCTAAGCCTGGCTCCCTGTGGTGGGGGCTGGAATGCCG |
| | | | | | | 4531 | |
| | | | | | 1231 | 72754 | CGGTTAGAACGCGCTCTACATCCGCTTTAAAGACAGAGGTCTAGACGTGA |
| | | | | | | 469 | |
| | | | | | 1232 | 41284 | CGGCCCCGCCCCCTGATCCGGTCCGGACTCCTAGGCCCGCCCCTACCTAA |
| | | | | | | 582 | |
| | | | | | 1233 | 26348 | CGCAAGTCATCACTGCAAAGCCTGGCCCAGCCATGGGAACAACTGCATTC |
| | | | | | | 74 | |
| | | | | | 1234 | 10162 | CTTCCCCAGCGTGGGGCGGAGCTCGTGGTGCTGGCCTGGAGGGACACACG |
| | | | | | | 79 | |
| | | | | | 1235 | 44318 | CGGTATTGGGAGAAAGTGAGGCACCTACAGGCTAAACCTGTGGCCAAACA |
| | | | | | | 844 | |
| | | | | | 1236 | 46999 | ACGCAGACCCCAGCTCAAGGCTGCACCGTTGCCATGGAGACGGTTGGGCG |
| | | | | | | 224 | |
| | | | | | 1237 | 88928 | CGAGCAGCCACTCTTGGAGAAATGGGGAATGGCGCTGTAAAAGCGCAGGC |
| | | | | | | 839 | |
| | | | | | 1238 | 11512 | TCATCTCTGAACCAGATACTAAAAATCCTCCAGGCGAGTGGAGAGGACCG |
| | | | | | | 9801 | |
| | | | | | 1239 | 37246 | CGCCAACCCTGAAGGTACGTCCCTCGCTCGGGTTTGCCCGTCCGTCCTTC |
| | | | | | | 827 | |
| | | | | | 1240 | 38035 | TCAGGGATGTGGACGATGGCCATCCCAAGACTGCCCCAGCCAAGGCACCG |
| | | | | | | 999 | |
| | | | | | 1241 | 95354 | GCTGCCTCCACTCTAGGATACAGATTCAGATTTTGGGAACCTGGGGTGCG |
| | | | | | | 741 | |
| | | | | | 1242 | 19617 | AAGCGCTTGCTTACGTAGCTGCGGAGCGAGCCTCTGCTTCATTACGAGCG |
| | | | | | | 873 | |
| | | | | | 1243 | 95026 | CGCCTCACGTCTGGCCTTGCAGAAGTCCGGAGTTCCGGCTCTGAACTGGA |
| | | | | | | 073 | |
| | | | | | 1244 | 19313 | TCCGGCAGGGAGCGGACCGTGGTGGAGGCCTCTGAGCCGAGCAGGCCACG |
| | | | | | | 76 | |
| | | | | | 1245 | 85824 | CGCGTATCTGAGAGCAACGTCTTTATCATCAGCACGGTGCGTTCCCTGCA |
| | | | | | | 216 | |
| | | | | | 1246 | 13412 | CGCCTTGCAGCGCAGCTCGGATCAGCAGCAGCCCAGGAGGCCTCCCGCCC |
| | | | | | | 5022 | |
| | | | | | 1247 | 26273 | TTACTTGGAGCTGGTGTACTTGGTGACCGCCTTGGTGCCCTCCGACACCG |
| | | | | | | 535 | |
| | | | | | 1248 | 63463 | TGAACCCCTCAAGTTTATAAATCACTTGGTAATCTTGGCCCTGCTTTTCG |
| | | | | | | 421 | |
| | | | | | 1249 | 10091 | AGAGCAAACGTCAGGGATTATTATGTCGATTTTCCGAGGACCAGGGTTCG |
| | | | | | | 1687 | |
| | | | | | 1250 | 31716 | CACTTAGCTAAAGCCATTTCCTATGCTCCTTCCAGACAGGTTTTGCAACG |
| | | | | | | 64 | |
| | | | | | 1251 | 32713 | CCATGGGTCTTTGGGTGTTGGAATAAAACCCAAACTGGACAAAGAGCTCG |
| | | | | | | 464 | |
| | | | | | 1252 | 27513 | TGAATGTCATCGAAGAAGCTCCTGATTTAGATTTTAACTGTACGTAAACG |
| | | | | | | 092 | |
| | | | | | 1253 | 21251 | CGGCCCTGCGCAGCTGCCCCATGTGCCAGAAGGAGTTCGCCCCCAGGTGA |
| | | | | | | 22 | |
| | | | | | 1254 | 11353 | TCAAATGGTGAAATCTGAGCTTCCAAAAATACCTACTGTCGAGCACCTCG |
| | | | | | | 7807 | |
| | | | | | 1255 | 20735 | CGCCCCGCAAGCGGGTCGGGCCCGCCCCAACAGCCCTCGGCTCTCCTCTC |
| | | | | | | 532 | |
| | | | | | 1256 | 13702 | ACAAGGTACCATCTCATGGCCTCTTGGTTCAAATCCGTTTTCTTGCTCCG |
| | | | | | | 8604 | |
| | | | | | 1257 | 26624 | CGCCTCTCCCGCCCGTGGGATGTCAGGTCTGTTGTCAGGCCCACGAAGGC |
| | | | | | | 667 | |
| | | | | | 1258 | 77734 | ATTAATCTATATCAAAAGGTCAGTCCTTAAGGGGTCTGCTCCATTTCTCG |
| | | | | | | 490 | |
| | | | | | 1259 | 60076 | GTAGGCAAGCGCAGGGGGCGGGCGGCCGAGGAGCGCTTTCGAATTAATCG |
| | | | | | | 70 | |
| | | | | | 1260 | 26758 | CTGGAGAATGCAGACGTGGATCCTGCAGGCCGCTGCCTCCCAAGTGAGCG |
| | | | | | | 166 | |
| | | | | | 1261 | 11205 | CGGCGCCAGGGCCCCCTCAGCGCTGCTCCTGGGCAGTCTCTGTGTCCTCC |
| | | | | | | 8304 | |
| | | | | | 1262 | 45098 | GCAGGGCTTCTATACCAGCAAGATTCATAAGCATGAGCAGATTTGATTCG |
| | | | | | | 028 | |
| | | | | | 1263 | 81188 | CGCCGGGACACAGGCTCACCTTCCTGAGCCCAGGCCACTGGGACCAAGCT |
| | | | | | | 405 | |
| | | | | | 1264 | 91036 | AGTGTGGCGACCTGGCAGCTGCAGCGGGACTCAGCTTTTGGGAACTGTCG |
| | | | | | | 888 | |
| | | | | | 1265 | 13662 | GGATTTGGTCTAGGCTTTCTGGCTTGGCCCTGGAACTCTCAGCAAGGACG |
| | | | | | | 9694 | |
| | | | | | 1266 | 68122 | CGTCCGGGGTTCGGCTGCCTCGACCTGCCCGATTTTCCGCTGCCCCCAAC |
| | | | | | | 139 | |
| | | | | | 1267 | 78176 | CGCAGCAAAACTTAGTTGAGTAGAAATAAGCTCCCGTATGAACACCGGGC |
| | | | | | | 622 | |
| | | | | | 1268 | 22518 | CTGATGAGCAGCTTGCCCGCGTAGGCGGTGGGCACGCAGCAGGGTGGGCG |
| | | | | | | 37 | |
| | | | | | 1269 | 46925 | AGTTCGAACTACTGGGGGAACTCAAGACTGCGTGCCTTAGACCTATCCCG |
| | | | | | | 181 | |
| | | | | | 1270 | 38821 | CGCGCAGACCGAGCAGACTGCCGCAGCGACTCATCCTATCGATAAGGTTC |
| | | | | | | 429 | |
| | | | | | 1271 | 27772 | CGCTGCGTCTTCTGCGGCGCAAATCTTTCGACCCTCTGCGCTCTGCCGGG |
| | | | | | | 805 | |
| | | | | | 1272 | 55243 | CGCTGAGTAATAAGTGTGAAGTTTAGAACACAAACCTTGCAAGAGCTTTC |
| | | | | | | 181 | |
| | | | | | 1273 | 15668 | AGCCGGGAAGGGGCGGGCCCTGGCTGCGGGAAGCCCCGTGGAGGGTAGCG |
| | | | | | | 60 | |
| | | | | | 1274 | 11030 | CGCCACGCGCACCAGATCGCGCTGCAGCAGCACTGCCTGGCGCCACCCCA |
| | | | | | | 1075 | |
| | | | | | 1275 | 36872 | CGCCCCACCCCCTGCCGCCTGCGGGCATCGCTTTATGACGTCACCACGTG |
| | | | | | | 048 | |
| | | | | | 1276 | 11023 | CGGGGCTCGGCCTCGCCAGCTTCCCTACCCAGCCCTGAGGCTCCACGAGT |
| | | | | | | 0252 | |
| | | | | | 1277 | 40618 | CGGAAGGTCTGGGAACTGGGAGCAGGAAAAATGAAAGCCGCCTATCTCCA |
| | | | | | | 356 | |
| | | | | | 1278 | 14713 | GTGTCAACAGGGCCAAGAGGATCCAGAAGAGTGGGCCTGCACTCTTTTCG |
| | | | | | | 9563 | |
| | | | | | 1279 | 15847 | CGGCCCTCACCCTGTTTTTGGTGTGGAATTCATCAGCCAGAGTGAAAGAG |
| | | | | | | 5854 | |
| | | | | | 1280 | 15169 | CGGCCAGAGAAATGGGCCGAAATTACTGCCAGACACATCCGACCTTAGTA |
| | | | | | | 4320 | |
| | | | | | 1281 | 15018 | CATTCGTGCTATTATAAATCAAACAGCAACAGGGCATGATTAAACACACG |
| | | | | | | 3516 | |
| | | | | | 1282 | 87967 | AATTGATCAGAAGTTAGACCGGAGGCTCTGTCAAGTCACGGCCACGGCCG |
| | | | | | | 845 | |
| | | | | | 1283 | 32223 | TTCTTCCCTGTGACTCAGCCCTGGAGGAATTTCCCTTTTGAAATCTTGCG |
| | | | | | | 114 | |
| | | | | | 1284 | 26271 | CGCAGACTAACCGAGAAGAGAGGATTCAAAAATGAAGTGATTCATCCAAT |
| | | | | | | 827 | |
| | | | | | 1285 | 30039 | ATCCCTCAGTCTCCGTCTCCTTTGCACACAGGAGTTGGGAGGCTTCAGCG |
| | | | | | | 113 | |
| | | | | | 1286 | 87038 | CGCCCTTGGACATTACTCTCTGGATACAACGGAGAGTGTGCCAAGGCTTA |
| | | | | | | 972 | |
| | | | | | 1287 | 10485 | CTTTGCCTGAGCCACAAGGGACCGGAGGAATGGGGTTCATTCCCACGCCG |
| | | | | | | 2151 | |
| | | | | | 1288 | 11471 | AATACAAAGTAAGCGAACTGTGCTTCCCCTACAGCTGCAAGCCCAGAGCG |
| | | | | | | 3108 | |
| | | | | | 1289 | 32410 | CATCTTTCACGTGGGGCAGCGGAGGCCTGGCGGGGACGGGGAGCCGCTCG |
| | | | | | | 614 | |
| | | | | | 1290 | 13910 | CGGGCCCTGGCAAAATGGGCTTTTATTCTGCTCAACAACAGGAGGGAAAG |
| | | | | | | 138 | |
| | | | | | 1291 | 15717 | CGCGCGATGCCTAGAAGTGCTCTGGTGTCACTCCCCTGTATAGGACCTGG |
| | | | | | | 8294 | |
| | | | | | 1292 | 18546 | CGCCCTAGAAGCGACCTGAATGGAAAAATCTGCACACGAACTAATGGGTT |
| | | | | | | 2408 | |
| | | | | | 1293 | 91295 | GCGGGCGGGGCATGGGGGTGGTCTGCGCGCTCTGGGCGGGCTGCAGAGCG |
| | | | | | | 346 | |
| | | | | | 1294 | 14852 | GGCCCCGTGGGTCAGGTGTTGCCCCTGAGAGATGAGCAAGGACCGGTCCG |
| | | | | | | 756 | |
| | | | | | 1295 | 23834 | CGCTCTCGGGGGCCTTTGGCTCCAGGCAACTTGGGGCAAGCGTCTCAGTT |
| | | | | | | 860 | |
| | | | | | 1296 | 15717 | CGCCTCTCTCTGTGGAAGACGCGCTTGTCTCAAGCCCCGATGCTCTCAAA |
| | | | | | | 8165 | |
| | | | | | 1297 | 47998 | CGGTCTCAGCTACAACGGCGTGCATGCCAATTACGCCTGCAGGCTACCGC |
| | | | | | | 999 | |
| | | | | | 1298 | 37509 | CCCAAGCAGGTGAGGCGCGGCACAGGGCGGAGACAGGTGAGGGGGCAAC |
| | | | | | | 52 | G |
| | | | | | 1299 | 70150 | CGGCCGAACTCGGCAGCTCCAACCCAACTCGGCTTAACTCCGCCTCACCG |
| | | | | | | 922 | |
| | | | | | 1300 | 11706 | CGGCTGCTCCTACTTCACTGTTCTCTGGCCCCGAGTTTGAGAAGAGGGGG |
| | | | | | | 9780 | |
| | | | | | 1301 | 13702 | AGCCCTCCGAGAAATCGTACGTTCCTCTCACTTACTTTGAGTTGGAAACG |
| | | | | | | 8198 | |
| | | | | | 1302 | 77591 | CGGCTGCGGACCACAAAGACCACAGAATCCCGAAGCCGATTCCTCAGACC |
| | | | | | | 661 | |
| | | | | | 1303 | 13091 | CGGGAAGAAACACAGAATCAAAGTGTTGCTCAAGGGTGCTTTCTGATCCA |
| | | | | | | 949 | |
| | | | | | 1304 | 31375 | CGCGGGTGCTGAACGCGCCCTTCTAAGCTGGTGTGTTAGCACCTGTTCAT |
| | | | | | | 861 | |
| | | | | | 1305 | 44324 | GCATCCCAGTGATTCTTGGGTTCCGCGTGTAGTTTCGGAAGGAGACATCG |
| | | | | | | 747 | |
| | | | | | 1306 | 21666 | TCATCAGAGTTCCCCCTTTTGTTCCAAGTCCCGGAGCAGACAAAGAGGCG |
| | | | | | | 666 | |
| | | | | | 1307 | 22093 | CGGGCCTCGTCCCTTAATTCTAGAAATAGCGATCGCGAAAGCTAAGTGCC |
| | | | | | | 960 | |
| | | | | | 1308 | 30923 | TAATTATCCCTAATGAGGGCCCAGCCCCGGAGCTTGTTAAAGGATTTCCG |
| | | | | | | 306 | |
| | | | | | 1309 | 18232 | CGCCCCAAGATAAGTTAGTTTCCCTGGCAGATTTCCAGCGAGCTGGCTCT |
| | | | | | | 2749 | |
| | | | | | 1310 | 86383 | CGCCGGCTGTCACCCGCACCGGATCTGCTTCCAGGGCTTGGTTTCGCTTT |
| | | | | | | 255 | |
| | | | | | 1311 | 19152 | CGAGGTTGGGCCTTTGCCTGAATCTGTTGCTATTGCTCCCCTTGCTACCG |
| | | | | | | 073 | |
| | | | | | 1312 | 79815 | ATCTGCGTGGGACACAGTGACTCCTAGAAGGGTATCCCAGGATTTTAGCG |
| | | | | | | 639 | |
| | | | | | 1313 | 43276 | CGCCGCTGCGGCTCAGTTCCGCCCCCTCCGCAGGCTCCACCCACGGCTCA |
| | | | | | | 570 | |
| | | | | | 1314 | 91295 | CGCTCCAGCGCTCTGCAGCCCGCCCAGAGCGCGCAGACCACCCCCATGCC |
| | | | | | | 338 | |
| | | | | | 1315 | 23997 | CGGGAAGCCTTTCTGCCGCCCAGGCCCACAGGTCTCGAGACGCACATGCA |
| | | | | | | 4654 | |
| | | | | | 1316 | 20692 | GATGACGCTTGATTGTCTGAAGTCCCCACTTCGTTCTGCCTCCGGAGACG |
| | | | | | | 092 | |
| | | | | | 1317 | 68084 | AATGCGCTCTCCAAATGGGGAGTCTGTTGGGGAGGCCCTGGCGGGAGCCG |
| | | | | | | 453 | |
| | | | | | 1318 | 11353 | CGACGCCTGCAGCTTCTCTGGCTTCCGTCCAGCCACGCTAACTGTCACAA |
| | | | | | | 996 | |
| | | | | | 1319 | 38821 | ACAGTTCCATTCTTTTCCCATCCTCCACCTTGGCTAACTGAACCTTATCG |
| | | | | | | 390 | |
| | | | | | 1320 | 86383 | GGGGATTTGCGTTTGGGGGTCAGCATATCCTCGCACTCTCCCAACATTCG |
| | | | | | | 713 | |
| | | | | | 1321 | 92525 | TTGCTGTTATTACACACACAGGCCTTCCTGTCTGGTCGTTAGAAAAGCCG |
| | | | | | | 005 | |
| | | | | | 1322 | 50816 | AGGGGAGCCTTCCCATTTTTCTGGCCTCAAAAGTAAAGCAGCATTAGACG |
| | | | | | | 963 | |
| | | | | | 1323 | 17694 | CGGGGAGCCCCGCAGAGCAAAACCAAGGAAGATGTCTTCTCTGCTCGTCT |
| | | | | | | 80 | |
| | | | | | 1324 | 19146 | CGGCAAAGCTACAGAGGGAAACTAAGTCACTCGCCTGCAAAACGAGGTTT |
| | | | | | | 441 | |
| | | | | | 1325 | 78173 | CGCAGGTTTGTCCCCAGTTGATAGTTCCGTGAGTTCCTCAATTATTCCTC |
| | | | | | | 200 | |
| | | | | | 1326 | 11513 | CGCGCCCCACTCCGCCCAAGGGGAGAGCCCCACGGACCATTGTGGCTTGC |
| | | | | | | 6258 | |
| | | | | | 1298 | 37509 | CCCAAGCAGGTGAGGCGCGGCACAGGGCGGAGACAGGTGAGGGGGCAAC |
| | | | | | | 52 | G |
| | | | | | 1299 | 70150 | CGGCCGAACTCGGCAGCTCCAACCCAACTCGGCTTAACTCCGCCTCACCG |
| | | | | | | 922 | |
| | | | | | 1300 | 11706 | CGGCTGCTCCTACTTCACTGTTCTCTGGCCCCGAGTTTGAGAAGAGGGGG |
| | | | | | | 9780 | |
| | | | | | 1301 | 8198 | AGCCCTCCGAGAAATCGTACGTTCCTCTCACTTACTTTGAGTTGGAAACG |
| | | | | | | 13702 | |
| | | | | | 1302 | 77591 | CGGCTGCGGACCACAAAGACCACAGAATCCCGAAGCCGATTCCTCAGACC |
| | | | | | | 661 | |
| | | | | | 1303 | 13091 | CGGGAAGAAACACAGAATCAAAGTGTTGCTCAAGGGTGCTTTCTGATCCA |
| | | | | | | 949 | |
| | | | | | 1304 | 31375 | CGCGGGTGCTGAACGCGCCCTTCTAAGCTGGTGTGTTAGCACCTGTTCAT |
| | | | | | | 861 | |
| | | | | | 1305 | 44324 | GCATCCCAGTGATTCTTGGGTTCCGCGTGTAGTTTCGGAAGGAGACATCG |
| | | | | | | 747 | |
| | | | | | 1306 | 21666 | TCATCAGAGTTCCCCCTTTTGTTCCAAGTCCCGGAGCAGACAAAGAGGCG |
| | | | | | | 666 | |
| | | | | | 1307 | 22093 | CGGGCCTCGTCCCTTAATTCTAGAAATAGCGATCGCGAAAGCTAAGTGCC |
| | | | | | | 960 | |
| | | | | | 1308 | 30923 | TAATTATCCCTAATGAGGGCCCAGCCCCGGAGCTTGTTAAAGGATTTCCG |
| | | | | | | 306 | |
| | | | | | 1309 | 18232 | CGCCCCAAGATAAGTTAGTTTCCCTGGCAGATTTCCAGCGAGCTGGCTCT |
| | | | | | | 2749 | |
| | | | | | 1310 | 86383 | CGCCGGCTGTCACCCGCACCGGATCTGCTTCCAGGGCTTGGTTTCGCTTT |
| | | | | | | 255 | |
| | | | | | 1311 | 19152 | CGAGGTTGGGCCTTTGCCTGAATCTGTTGCTATTGCTCCCCTTGCTACCG |
| | | | | | | 073 | |
| | | | | | 1312 | 79815 | ATCTGCGTGGGACACAGTGACTCCTAGAAGGGTATCCCAGGATTTTAGCG |
| | | | | | | 639 | |
| | | | | | 1313 | 43276 | CGCCGCTGCGGCTCAGTTCCGCCCCCTCCGCAGGCTCCACCCACGGCTCA |
| | | | | | | 570 | |
| | | | | | 1314 | 91295 | CGCTCCAGCGCTCTGCAGCCCGCCCAGAGCGCGCAGACCACCCCCATGCC |
| | | | | | | 338 | |
| | | | | | 1315 | 23997 | CGGGAAGCCTTTCTGCCGCCCAGGCCCACAGGTCTCGAGACGCACATGCA |
| | | | | | | 4654 | |
| | | | | | 1316 | 20692 | GATGACGCTTGATTGTCTGAAGTCCCCACTTCGTTCTGCCTCCGGAGACG |
| | | | | | | 092 | |
| | | | | | 1317 | 68084 | AATGCGCTCTCCAAATGGGGAGTCTGTTGGGGAGGCCCTGGCGGGAGCCG |
| | | | | | | 453 | |
| | | | | | 1318 | 11353 | CGACGCCTGCAGCTTCTCTGGCTTCCGTCCAGCCACGCTAACTGTCACAA |
| | | | | | | 996 | |
| | | | | | 1319 | 38821 | ACAGTTCCATTCTTTTCCCATCCTCCACCTTGGCTAACTGAACCTTATCG |
| | | | | | | 390 | |
| | | | | | 1320 | 86383 | GGGGATTTGCGTTTGGGGGTCAGCATATCCTCGCACTCTCCCAACATTCG |
| | | | | | | 713 | |
| | | | | | 1321 | 92525 | TTGCTGTTATTACACACACAGGCCTTCCTGTCTGGTCGTTAGAAAAGCCG |
| | | | | | | 005 | |
| | | | | | 1322 | 50816 | AGGGGAGCCTTCCCATTTTTCTGGCCTCAAAAGTAAAGCAGCATTAGACG |
| | | | | | | 963 | |
| | | | | | 1323 | 17694 | CGGGGAGCCCCGCAGAGCAAAACCAAGGAAGATGTCTTCTCTGCTCGTCT |
| | | | | | | 80 | |
| | | | | | 1324 | 19146 | CGGCAAAGCTACAGAGGGAAACTAAGTCACTCGCCTGCAAAACGAGGTTT |
| | | | | | | 441 | |
| | | | | | 1325 | 78173 | CGCAGGTTTGTCCCCAGTTGATAGTTCCGTGAGTTCCTCAATTATTCCTC |
| | | | | | | 200 | |
| | | | | | 1326 | 11513 | CGCGCCCCACTCCGCCCAAGGGGAGAGCCCCACGGACCATTGTGGCTTGC |
| | | | | | | 6258 | |
| | | | | | 1298 | 37509 | CCCAAGCAGGTGAGGCGCGGCACAGGGCGGAGACAGGTGAGGGGGCAAC |
| | | | | | | 52 | G |
| | | | | | 1299 | 70150 | CGGCCGAACTCGGCAGCTCCAACCCAACTCGGCTTAACTCCGCCTCACCG |
| | | | | | | 922 | |
| | | | | | 1300 | 11706 | CGGCTGCTCCTACTTCACTGTTCTCTGGCCCCGAGTTTGAGAAGAGGGGG |
| | | | | | | 9780 | |
| | | | | | 1301 | 13702 | AGCCCTCCGAGAAATCGTACGTTCCTCTCACTTACTTTGAGTTGGAAACG |
| | | | | | | 8198 | |
| | | | | | 1302 | 77591 | CGGCTGCGGACCACAAAGACCACAGAATCCCGAAGCCGATTCCTCAGACC |
| | | | | | | 661 | |
| | | | | | 1303 | 13091 | CGGGAAGAAACACAGAATCAAAGTGTTGCTCAAGGGTGCTTTCTGATCCA |
| | | | | | | 949 | |
| | | | | | 1304 | 31375 | CGCGGGTGCTGAACGCGCCCTTCTAAGCTGGTGTGTTAGCACCTGTTCAT |
| | | | | | | 861 | |
| | | | | | 1305 | 44324 | GCATCCCAGTGATTCTTGGGTTCCGCGTGTAGTTTCGGAAGGAGACATCG |
| | | | | | | 747 | |
| | | | | | 1306 | 21666 | TCATCAGAGTTCCCCCTTTTGTTCCAAGTCCCGGAGCAGACAAAGAGGCG |
| | | | | | | 666 | |
| | | | | | 1307 | 22093 | CGGGCCTCGTCCCTTAATTCTAGAAATAGCGATCGCGAAAGCTAAGTGCC |
| | | | | | | 960 | |
| | | | | | 1308 | 30923 | TAATTATCCCTAATGAGGGCCCAGCCCCGGAGCTTGTTAAAGGATTTCCG |
| | | | | | | 306 | |
| | | | | | 1309 | 18232 | CGCCCCAAGATAAGTTAGTTTCCCTGGCAGATTTCCAGCGAGCTGGCTCT |
| | | | | | | 2749 | |
| | | | | | 1310 | 86383 | CGCCGGCTGTCACCCGCACCGGATCTGCTTCCAGGGCTTGGTTTCGCTTT |
| | | | | | | 255 | |
| | | | | | 1311 | 19152 | CGAGGTTGGGCCTTTGCCTGAATCTGTTGCTATTGCTCCCCTTGCTACCG |
| | | | | | | 073 | |
| | | | | | 1312 | 79815 | ATCTGCGTGGGACACAGTGACTCCTAGAAGGGTATCCCAGGATTTTAGCG |
| | | | | | | 639 | |
| | | | | | 1313 | 43276 | CGCCGCTGCGGCTCAGTTCCGCCCCCTCCGCAGGCTCCACCCACGGCTCA |
| | | | | | | 570 | |
| | | | | | 1314 | 91295 | CGCTCCAGCGCTCTGCAGCCCGCCCAGAGCGCGCAGACCACCCCCATGCC |
| | | | | | | 338 | |
| | | | | | 1315 | 23997 | CGGGAAGCCTTTCTGCCGCCCAGGCCCACAGGTCTCGAGACGCACATGCA |
| | | | | | | 4654 | |
| | | | | | 1316 | 20692 | GATGACGCTTGATTGTCTGAAGTCCCCACTTCGTTCTGCCTCCGGAGACG |
| | | | | | | 092 | |
| | | | | | 1317 | 68084 | AATGCGCTCTCCAAATGGGGAGTCTGTTGGGGAGGCCCTGGCGGGAGCCG |
| | | | | | | 453 | |
| | | | | | 1318 | 11353 | CGACGCCTGCAGCTTCTCTGGCTTCCGTCCAGCCACGCTAACTGTCACAA |
| | | | | | | 996 | |
| | | | | | 1319 | 38821 | ACAGTTCCATTCTTTTCCCATCCTCCACCTTGGCTAACTGAACCTTATCG |
| | | | | | | 390 | |
| | | | | | 1320 | 86383 | GGGGATTTGCGTTTGGGGGTCAGCATATCCTCGCACTCTCCCAACATTCG |
| | | | | | | 713 | |
| | | | | | 1321 | 92525 | TTGCTGTTATTACACACACAGGCCTTCCTGTCTGGTCGTTAGAAAAGCCG |
| | | | | | | 005 | |
| | | | | | 1322 | 50816 | AGGGGAGCCTTCCCATTTTTCTGGCCTCAAAAGTAAAGCAGCATTAGACG |
| | | | | | | 963 | |
| | | | | | 1323 | 17694 | CGGGGAGCCCCGCAGAGCAAAACCAAGGAAGATGTCTTCTCTGCTCGTCT |
| | | | | | | 80 | |
| | | | | | 1324 | 19146 | CGGCAAAGCTACAGAGGGAAACTAAGTCACTCGCCTGCAAAACGAGGTTT |
| | | | | | | 441 | |
| | | | | | 1325 | 78173 | CGCAGGTTTGTCCCCAGTTGATAGTTCCGTGAGTTCCTCAATTATTCCTC |
| | | | | | | 200 | |
| | | | | | 1326 | 11513 | CGCGCCCCACTCCGCCCAAGGGGAGAGCCCCACGGACCATTGTGGCTTGC |
| | | | | | | 6258 | |
| | | | | | 1298 | 37509 | CCCAAGCAGGTGAGGCGCGGCACAGGGCGGAGACAGGTGAGGGGGCAAC |
| | | | | | | 52 | G |
| | | | | | 1299 | 70150 | CGGCCGAACTCGGCAGCTCCAACCCAACTCGGCTTAACTCCGCCTCACCG |
| | | | | | | 922 | |
| | | | | | 1300 | 11706 | CGGCTGCTCCTACTTCACTGTTCTCTGGCCCCGAGTTTGAGAAGAGGGGG |
| | | | | | | 9780 | |
| | | | | | 1301 | 13702 | AGCCCTCCGAGAAATCGTACGTTCCTCTCACTTACTTTGAGTTGGAAACG |
| | | | | | | 19 | |
| | | | | | 1302 | 77591 | CGGCTGCGGACCACAAAGACCACAGAATCCCGAAGCCGATTCCTCAGACC |
| | | | | | | 661 | |
| | | | | | 1303 | 13091 | CGGGAAGAAACACAGAATCAAAGTGTTGCTCAAGGGTGCTTTCTGATCCA |
| | | | | | | 949 | |
| | | | | | 1304 | 31375 | CGCGGGTGCTGAACGCGCCCTTCTAAGCTGGTGTGTTAGCACCTGTTCAT |
| | | | | | | 861 | |
| | | | | | 1305 | 44324 | GCATCCCAGTGATTCTTGGGTTCCGCGTGTAGTTTCGGAAGGAGACATCG |
| | | | | | | 747 | |
| | | | | | 1306 | 21666 | TCATCAGAGTTCCCCCTTTTGTTCCAAGTCCCGGAGCAGACAAAGAGGCG |
| | | | | | | 666 | |
| | | | | | 1307 | 22093 | CGGGCCTCGTCCCTTAATTCTAGAAATAGCGATCGCGAAAGCTAAGTGCC |
| | | | | | | 960 | |
| | | | | | 1308 | 30923 | TAATTATCCCTAATGAGGGCCCAGCCCCGGAGCTTGTTAAAGGATTTCCG |
| | | | | | | 306 | |
| | | | | | 1309 | 18232 | CGCCCCAAGATAAGTTAGTTTCCCTGGCAGATTTCCAGCGAGCTGGCTCT |
| | | | | | | 2749 | |
| | | | | | 1310 | 86383 | CGCCGGCTGTCACCCGCACCGGATCTGCTTCCAGGGCTTGGTTTCGCTTT |
| | | | | | | 255 | |
| | | | | | 1311 | 19152 | CGAGGTTGGGCCTTTGCCTGAATCTGTTGCTATTGCTCCCCTTGCTACCG |
| | | | | | | 073 | |
| | | | | | 1312 | 79815 | ATCTGCGTGGGACACAGTGACTCCTAGAAGGGTATCCCAGGATTTTAGCG |
| | | | | | | 639 | |
| | | | | | 1313 | 43276 | CGCCGCTGCGGCTCAGTTCCGCCCCCTCCGCAGGCTCCACCCACGGCTCA |
| | | | | | | 570 | |
| | | | | | 1314 | 91295 | CGCTCCAGCGCTCTGCAGCCCGCCCAGAGCGCGCAGACCACCCCCATGCC |
| | | | | | | 338 | |
| | | | | | 1315 | 23997 | CGGGAAGCCTTTCTGCCGCCCAGGCCCACAGGTCTCGAGACGCACATGCA |
| | | | | | | 4654 | |
| | | | | | 1316 | 20692 | GATGACGCTTGATTGTCTGAAGTCCCCACTTCGTTCTGCCTCCGGAGACG |
| | | | | | | 092 | |
| | | | | | 1317 | 68084 | AATGCGCTCTCCAAATGGGGAGTCTGTTGGGGAGGCCCTGGCGGGAGCCG |
| | | | | | | 453 | |
| | | | | | 1318 | 11353 | CGACGCCTGCAGCTTCTCTGGCTTCCGTCCAGCCACGCTAACTGTCACAA |
| | | | | | | 996 | |
| | | | | | 1319 | 38821 | ACAGTTCCATTCTTTTCCCATCCTCCACCTTGGCTAACTGAACCTTATCG |
| | | | | | | 390 | |
| | | | | | 1320 | 86383 | GGGGATTTGCGTTTGGGGGTCAGCATATCCTCGCACTCTCCCAACATTCG |
| | | | | | | 713 | |
| | | | | | 1321 | 92525 | TTGCTGTTATTACACACACAGGCCTTCCTGTCTGGTCGTTAGAAAAGCCG |
| | | | | | | 005 | |
| | | | | | 1322 | 50816 | AGGGGAGCCTTCCCATTTTTCTGGCCTCAAAAGTAAAGCAGCATTAGACG |
| | | | | | | 963 | |
| | | | | | 1323 | 17694 | CGGGGAGCCCCGCAGAGCAAAACCAAGGAAGATGTCTTCTCTGCTCGTCT |
| | | | | | | 80 | |
| | | | | | 1324 | 19146 | CGGCAAAGCTACAGAGGGAAACTAAGTCACTCGCCTGCAAAACGAGGTTT |
| | | | | | | 441 | |
| | | | | | 1325 | 78173 | CGCAGGTTTGTCCCCAGTTGATAGTTCCGTGAGTTCCTCAATTATTCCTC |
| | | | | | | 200 | |
| | | | | | 1326 | 11513 | CGCGCCCCACTCCGCCCAAGGGGAGAGCCCCACGGACCATTGTGGCTTGC |
| | | | | | | 6258 | |
| | | | | | 1298 | 37509 | CCCAAGCAGGTGAGGCGCGGCACAGGGCGGAGACAGGTGAGGGGGCAAC |
| | | | | | | 52 | G |
| | | | | | 1299 | 70150 | CGGCCGAACTCGGCAGCTCCAACCCAACTCGGCTTAACTCCGCCTCACCG |
| | | | | | | 922 | |
| | | | | | 1300 | 11706 | CGGCTGCTCCTACTTCACTGTTCTCTGGCCCCGAGTTTGAGAAGAGGGGG |
| | | | | | | 9780 | |
| | | | | | 1301 | 13702 | AGCCCTCCGAGAAATCGTACGTTCCTCTCACTTACTTTGAGTTGGAAACG |
| | | | | | | 8198 | |
| | | | | | 1302 | 77591 | CGGCTGCGGACCACAAAGACCACAGAATCCCGAAGCCGATTCCTCAGACC |
| | | | | | | 661 | |
| | | | | | 1303 | 13091 | CGGGAAGAAACACAGAATCAAAGTGTTGCTCAAGGGTGCTTTCTGATCCA |
| | | | | | | 949 | |
| | | | | | 1304 | 31375 | CGCGGGTGCTGAACGCGCCCTTCTAAGCTGGTGTGTTAGCACCTGTTCAT |
| | | | | | | 861 | |
| | | | | | 1305 | 44324 | GCATCCCAGTGATTCTTGGGTTCCGCGTGTAGTTTCGGAAGGAGACATCG |
| | | | | | | 747 | |
| | | | | | 1306 | 21666 | TCATCAGAGTTCCCCCTTTTGTTCCAAGTCCCGGAGCAGACAAAGAGGCG |
| | | | | | | 666 | |
| | | | | | 1307 | 22093 | CGGGCCTCGTCCCTTAATTCTAGAAATAGCGATCGCGAAAGCTAAGTGCC |
| | | | | | | 960 | |
| | | | | | 1308 | 30923 | TAATTATCCCTAATGAGGGCCCAGCCCCGGAGCTTGTTAAAGGATTTCCG |
| | | | | | | 306 | |
| | | | | | 1309 | 18232 | CGCCCCAAGATAAGTTAGTTTCCCTGGCAGATTTCCAGCGAGCTGGCTCT |
| | | | | | | 2749 | |
| | | | | | 1310 | 86383 | CGCCGGCTGTCACCCGCACCGGATCTGCTTCCAGGGCTTGGTTTCGCTTT |
| | | | | | | 255 | |
| | | | | | 1311 | 19152 | CGAGGTTGGGCCTTTGCCTGAATCTGTTGCTATTGCTCCCCTTGCTACCG |
| | | | | | | 073 | |
| | | | | | 1312 | 79815 | ATCTGCGTGGGACACAGTGACTCCTAGAAGGGTATCCCAGGATTTTAGCG |
| | | | | | | 639 | |
| | | | | | 1313 | 43276 | CGCCGCTGCGGCTCAGTTCCGCCCCCTCCGCAGGCTCCACCCACGGCTCA |
| | | | | | | 570 | |
| | | | | | 1314 | 91295 | CGCTCCAGCGCTCTGCAGCCCGCCCAGAGCGCGCAGACCACCCCCATGCC |
| | | | | | | 338 | |
| | | | | | 1315 | 23997 | CGGGAAGCCTTTCTGCCGCCCAGGCCCACAGGTCTCGAGACGCACATGCA |
| | | | | | | 4654 | |
| | | | | | 1316 | 20692 | GATGACGCTTGATTGTCTGAAGTCCCCACTTCGTTCTGCCTCCGGAGACG |
| | | | | | | 092 | |
| | | | | | 1317 | 68084 | AATGCGCTCTCCAAATGGGGAGTCTGTTGGGGAGGCCCTGGCGGGAGCCG |
| | | | | | | 453 | |
| | | | | | 1318 | 11353 | CGACGCCTGCAGCTTCTCTGGCTTCCGTCCAGCCACGCTAACTGTCACAA |
| | | | | | | 996 | |
| | | | | | 1319 | 38821 | ACAGTTCCATTCTTTTCCCATCCTCCACCTTGGCTAACTGAACCTTATCG |
| | | | | | | 390 | |
| | | | | | 1320 | 86383 | GGGGATTTGCGTTTGGGGGTCAGCATATCCTCGCACTCTCCCAACATTCG |
| | | | | | | 713 | |
| | | | | | 1321 | 92525 | TTGCTGTTATTACACACACAGGCCTTCCTGTCTGGTCGTTAGAAAAGCCG |
| | | | | | | 005 | |
| | | | | | 1322 | 50816 | AGGGGAGCCTTCCCATTTTTCTGGCCTCAAAAGTAAAGCAGCATTAGACG |
| | | | | | | 963 | |
| | | | | | 1323 | 17694 | CGGGGAGCCCCGCAGAGCAAAACCAAGGAAGATGTCTTCTCTGCTCGTCT |
| | | | | | | 80 | |
| | | | | | 1324 | 19146 | CGGCAAAGCTACAGAGGGAAACTAAGTCACTCGCCTGCAAAACGAGGTTT |
| | | | | | | 441 | |
| | | | | | 1325 | 78173 | CGCAGGTTTGTCCCCAGTTGATAGTTCCGTGAGTTCCTCAATTATTCCTC |
| | | | | | | 200 | |
| | | | | | 1326 | 11513 | CGCGCCCCACTCCGCCCAAGGGGAGAGCCCCACGGACCATTGTGGCTTGC |
| | | | | | | 6258 | |
| | | | | | 1327 | 10823 | CGGGCCTCAAGTCCCTGCCCGCCATGCCCCAGGGCCCTCAGCTCCACCAC |
| | | | | | | 8461 | |
| | | | | | 1328 | 40441 | AATAGCGTTACTTGTGCAACACAACATATATAATGGAAAAGGCCGATGCG |
| | | | | | | 216 | |
| | | | | | 1329 | 35384 | GTCGTTGCCATGACACCGCGGGCAGGTGGGCGGCTCTGGCGGCTGTTTCG |
| | | | | | | 669 | |
| | | | | | 1330 | 61881 | CGCGCACCACGTACTGCGCCACCTTGAAGGAGCTCAGGTACTCGTTCATG |
| | | | | | | 20 | |
| | | | | | 1331 | 30037 | CGCCTCCAGACCTCGGTTCCCGCTCCAGCACGTTAAGCAGGGCGCACTCA |
| | | | | | | 833 | |
| | | | | | 1332 | 31498 | CAAGAAGGAGGTCAGCCGGGTGCTGTGCAAGCGGTGCGGTAAGTTCCTCG |
| | | | | | | 256 | |
| | | | | | 1333 | 92979 | GCCCTGGCTCTGGTCTGAGGTTGCCCTTGAACGATTTCGAGGCTCCCTCG |
| | | | | | | 709 | |
| | | | | | 1334 | 15136 | CTTACTTAGCGTGCGAGACAGGCTGGTAGTGCGAGCGGCCAGTGGTGGCG |
| | | | | | | 505 | |
| | | | | | 1335 | 54154 | CGCGGCCTGACCCCCGAGGCAAGGGGTCCCAGGAGTTGACCCTCAAATTG |
| | | | | | | 746 | |
| | | | | | 1336 | 24837 | CGGCCCAGCCGGGAGGCGGCGCACAGCCAACAGCACCCCCACCCCCACCC |
| | | | | | | 892 | |
| | | | | | 1337 | 52300 | CGGCCGCCAGACGTTGCCTACAGTCTCGGCTCTGTCTCCCACGGCTGTGG |
| | | | | | | 911 | |
| | | | | | 1338 | 41071 | GAGCATGCTCAGTTCATATGACTGATTTTCACTAAGTGTAACAATCAACG |
| | | | | | | 405 | |
| | | | | | 1339 | 13012 | CTCAGACCCAGCAGGAAGGGCGAGCGCACTGAGGCTGGGAAACGCAGGC |
| | | | | | | 3952 | G |
| | | | | | 1340 | 89952 | CGCCATGAGTCATGCAAGGACTGGGCGACAGAAGCTCTGAGCTTCTATTT |
| | | | | | | 272 | |
| | | | | | 1341 | 33292 | CGGCGCGGATGCCCCAGCAGCCCCCGCTCCACTACGCCAAGCCCTCGCCC |
| | | | | | | 92 | |
| | | | | | 1342 | 18006 | CGGGCGGGCCGCTGGAGTTTATCAGCTCCAGGACAAATCTGCCTCTGACC |
| | | | | | | 06 | |
| | | | | | 1343 | 10190 | TACCGAGAGACGGCTGTGGGGGAGGCTCCTGCGGGAGAAATGGCCAAACG |
| | | | | | | 6886 | |
| | | | | | 1344 | 39471 | CGGTGACGAAGCTTCGAGCAGCGCCGAACAGCCCCAGCCCCAGCTCCCGC |
| | | | | | | 028 | |
| | | | | | 1345 | 11897 | CGAAGCCTTCTAGATAAATACTGTGTAGAATATGGTGTGGGTGTCATTGG |
| | | | | | | 5504 | |
| | | | | | 1346 | 13665 | CGTGCGCGCTAAAAACCCACTTCAAAGTCTGTTCCCGGCACGGGGGCGGC |
| | | | | | | 6581 | |
| | | | | | 1347 | 81185 | AGTGGTAGGCAGGATGTAGACCAGGGCGGGCGCAGCCCTGGCGCATAGCG |
| | | | | | | 228 | |
| | | | | | 1348 | 81188 | CCTTCCTGAGCCCAGGCCACTGGGACCAAGCTGATACTCAGAAACAGCCG |
| | | | | | | 471 | |
| | | | | | 1349 | 21666 | TCTGCTGTCAGGCCGGGAAAAAGTGTCCGAACGCCTCGTGGACTGCAGCG |
| | | | | | | 834 | |
| | | | | | 1350 | 47468 | CGCTCTGTCTGTTCGGAACAGATGTCGGCCCTCGGAGGGGTTTCTCTGCC |
| | | | | | | 154 | |
| | | | | | 1351 | 67326 | CGCTCCGCGGCGGAGGCGACCCAGTTTCCCAGCTCTCGTCCTCGCCACTT |
| | | | | | | 082 | |
| | | | | | 1352 | 14060 | CGAAGCTCACCGTGCCAGCCTATGTACTGTTTTCGTCTTATCCTGTAGCG |
| | | | | | | 2851 | |
| | | | | | 1353 | 26598 | ACCTGGCGCTCAAAGGCGAGGTGCTCGCTGAGGTGGGACCAGGTGAAACG |
| | | | | | | 502 | |
| | | | | | 1354 | 41158 | CGTAGTTACTTGTATTGCTTTACATTTGCTCGTCCCAGTGTGTGAGGGCC |
| | | | | | | 986 | |
| | | | | | 1355 | 19788 | CTGTGGTACCAAAGGATCTACACAGCTATGTTTGGCCGAAATAATTTCCG |
| | | | | | | 7955 | |
| | | | | | 1356 | 67134 | AATTTGGTGCACAAGAGGTGCACAGCAAACGCCTGTGGAATGAGTGAACG |
| | | | | | | 587 | |
| | | | | | 1357 | 29644 | CGAACCTGTACAGAAGACGCTCCCATGGGTGGGCGAGGTAGCTGCCACCC |
| | | | | | | 756 | |
| | | | | | 1358 | 17520 | CGCGAGGAGCAAATGGAACAGCACAAGCTGCCCGCAGCCCGCACCATCGC |
| | | | | | | 0071 | |
| | | | | | 1359 | 13675 | ATATCAAAGCTGGGAGAGACTCCGGCTCTGCATATGAGCGACTCTGTTCG |
| | | | | | | 1656 | |
| | | | | | 1360 | 76101 | GCACATGACCAGAGGCGGCCGGTAATCAGGGCAGAGTCTGGTGTGGACCG |
| | | | | | | 391 | |
| | | | | | 1361 | 10451 | CGGCTGGGTTAATTCATGATGTTTGCCCTTTTAATAAAATATAAGGGTAG |
| | | | | | | 3155 | |
| | | | | | 1362 | 22931 | CGCGTTTCCCGGACAGTGAGGCAGGACCTAAACTCTTCCGTTAACACTAC |
| | | | | | | 73 | |
| | | | | | 1363 | 57345 | CGGAGCTTGTAGCGGGTAGGGACTCCACACACCAGGGAACCCTACAGAAC |
| | | | | | | 71 | |
| | | | | | 1364 | 11513 | CGGGCTCAGGACGCCTAGGCGTCCAAGCTGTGTTCCCCACCGCGCCCCAC |
| | | | | | | 6298 | |
| | | | | | 1365 | 11207 | CAGCCCGCCAGGGTGTCACTGGAGACAGAATGGAGGTGCTGCCGGACTCG |
| | | | | | | 3613 | |
| | | | | | 1366 | 91580 | GTGCTTCCCTCAAAAGGGCATAAAGGCCCCCTTTCTTCTGCTCTCCTGCG |
| | | | | | | 507 | |
| | | | | | 1367 | 14194 | TCATCCCCCCGGGCGCTGAGTCCTTCATGTCCTATGGGCTGTGTAGGACG |
| | | | | | | 3158 | |
| | | | | | 1368 | 32325 | CATAGCATTGCCTGGCTGGGAAGTTCAATGTGTTCACAGCCAATACCACG |
| | | | | | | 334 | |
| | | | | | 1369 | 15600 | CGTGAATACTCTGTGACTGGCTTGCTGAAACACAGCTTGAAATCCCTGTT |
| | | | | | | 8770 | |
| | | | | | 1370 | 53208 | CGGGGTCTGGTGGTGTGCACTCTGCTGCTCCATTTGCTGTCCAGTTGTTT |
| | | | | | | 762 | |
| | | | | | 1371 | 46387 | ACAGAGAGCCGCAGGGGCCGGCTCTGGGTACTGCTGGGGCAGGGCCTCCG |
| | | | | | | 992 | |
| | | | | | 1372 | 73247 | CGGGCATACGCTGTTTTCTGTATATGGGTTTACCCTCCAACCCATATACA |
| | | | | | | 613 | |
| | | | | | 1373 | 28549 | CTGTCCTTCCCTGAGCAGCCGGGCCTAGAGTCAAGGCGCGATGATTGCCG |
| | | | | | | 922 | |
| | | | | | 1374 | 50556 | CGGCTCACAGTGTTCCAGACCCCCCTTAAGGGTGAAGCCCCAGGGTGCCC |
| | | | | | | 917 | |
| | | | | | 1375 | 11513 | GTCTAGAATCAATACGGCCCAGACAGCCCTTCACAAAGGGGCTCCAGGCG |
| | | | | | | 6060 | |
| | | | | | 1376 | 14953 | CGCCAGCCGCCAGCTGCTGAGTCACTTTTGTCAAAGAGTGGCCTCGGCCC |
| | | | | | | 5695 | |
| | | | | | 1377 | 18592 | GTGAAGCATCTCATAAACCACTGCCTTTCTGATATTGGTCTCTTTGTTCG |
| | | | | | | 5146 | |
| | | | | | 1378 | 17013 | GACTCAGTTCCTATTAGGTGTTCCGGTTTGTTCATTCTCAAAGGGACCCG |
| | | | | | | 7321 | |
| | | | | | 1379 | 11356 | CGTTGAGAGGACGTCAACGAATCTATTTGTTGGGCTGAATCCTTCCCCAG |
| | | | | | | 8090 | |
| | | | | | 1380 | 13781 | GTTTCTGGCGAGCAGGAGTGTGGCGATCTTGGAGAGCTTGCGCACCGACG |
| | | | | | | 4960 | |
| | | | | | 1381 | 24769 | CTCGGCGCGGTCTTGGTCTGGGTCGGTGGGGTTGGGCCGGGAGACCCGCG |
| | | | | | | 4271 | |
| | | | | | 1382 | 44324 | CGCCTACGCACTCGGAGCACAGGTTACAACTTCCAGAAACCCTTGGGACG |
| | | | | | | 951 | |
| | | | | | 1383 | 36021 | ACAATTTCAAGTGTTGACAGCATCTGCTAAGTCTTCAGAATCTCAGCTCG |
| | | | | | | 943 | |
| | | | | | 1384 | 42955 | GAGGCAGAGCTGCCTCCACACCCGGTGCCAGGGGGTTGGGGTTGGCAGCG |
| | | | | | | 658 | |
| | | | | | 1385 | 74020 | TCCGGGAACTGGTGCACGCAAGAGCTTAACTGATTTCGCCGCGGTCTGCG |
| | | | | | | 976 | |
| | | | | | 1386 | 11913 | GCATGAGACTCTCTGGTTGTGTTCCGTTCGCACTCAAAGTCCTCGAATCG |
| | | | | | | 4684 | |
| | | | | | 1387 | 32138 | CCAGCAGCCCAAGACAGCATGAGAGGTGCTAGCCCAAATATTGACCTACG |
| | | | | | | 937 | |
| | | | | | 1388 | 35479 | CGCAGCCCACACCCTGGGGGTCCACCCGGGCTCTGCACCCCGCCCACCTC |
| | | | | | | 910 | |
| | | | | | 1389 | 10111 | GACGGATTTGCAAAAGCAGGCGAGTCGTGCAGAGGCTCCTAAAATGCCCG |
| | | | | | | 1261 | |
| | | | | | 1390 | 14034 | GGGAGGGCCTACGGAGGGAGCGGCCCCAGGCGCTTTCTAGAGCGTGAGCG |
| | | | | | | 6236 | |
| | | | | | 1391 | 36977 | GAGATTTCAAGTGTCCACACTTCTAGTTTAGACATTTGGCAACCAATTCG |
| | | | | | | 887 | |
| | | | | | 1392 | 14709 | CGCGTTCAAAGATTTTGTTTTCACGGCTGAGGGGGCTTTTTTCCAATGTA |
| | | | | | | 8568 | |
| | | | | | 1393 | 10090 | CGCAGAAAGGGAGCCCACTGAGACCGCAGGGGCTGACAAATCACTGCTAA |
| | | | | | | 9443 | |
| | | | | | 1394 | 54935 | GACCTGAGCCACTGCACCCGGCTAAGCGTTCCTTTTTTTAAAAAAAATCG |
| | | | | | | 605 | |
| | | | | | 1395 | 24583 | CGATGTTAGGGGCGTTTTTCATTTTGATGTTGGTTTTTGATATTACCACC |
| | | | | | | 375 | |
| | | | | | 1396 | 92946 | CGCAGGCCACTATAGGAGGCAGAACAGTAAACAGATCATTGAAAAGACCT |
| | | | | | | 825 | |
| | | | | | 1397 | 13867 | TGTTCCAGGCCTGACGGGTGTTCAGGGTGTAGGACTCAGGGTGTAGGACG |
| | | | | | | 9347 | |
| | | | | | 1398 | 32120 | GCCTCTGTACTGCTCTGTAGTCTCTGCTAGGACATGGACGAAAAGGGACG |
| | | | | | | 773 | |
| | | | | | 1399 | 16100 | CTAGCTGGGAGGTGATGGGGACTGCCTGTCATTCCTGTCAGTCTCTCACG |
| | | | | | | 7634 | |
| | | | | | 1400 | 26674 | ATCATTCTCCGGACTTTAAAGGACAAACTAAATGGAGACCCACCAATGCG |
| | | | | | | 046 | |
| | | | | | 1401 | 28549 | TCCACAAAGCTGCCCCCACTTCGTAAGGGCCTGTGCCTGGAGGGTCGTCG |
| | | | | | | 698 | |
| | | | | | 1402 | 67600 | CGGTTGCCGAGCTCTGCCTACCTCTGAAAGGCCTAGAGAGCAGAGTTCTC |
| | | | | | | 707 | |
| | | | | | 1403 | 11381 | CGCTCTTGGGCCAAAAGCGGGATGAGGGGAGGAGCCAAGAGCAGCCAATC |
| | | | | | | 8537 | |
| | | | | | 1404 | 44083 | CTTGACTGAACTAGCTTCTTATCTGTATGATAGAGAAACATGCCAAAGCG |
| | | | | | | 518 | |
| | | | | | 1405 | 20145 | TCCCAGGACTTACAATGTTTGCTAAGACGCAAGAGGGTGTGACACAGACG |
| | | | | | | 0527 | |
| | | | | | 1406 | 17266 | CGCCAATTATTGCAGCGCCGCAGCCCCTTTGTTGATACAGTAGTCCGAAA |
| | | | | | | 5707 | |
| | | | | | 1407 | 12425 | GTTTCACCTCCACTGCCCTGGAGGTAGAACTGGTGGTGAAGGTATCTACG |
| | | | | | | 4360 | |
| | | | | | 1408 | 31883 | CGGCCGCAGTGAGCCAGCTGCAGCCTGGGAGCACAGTGTGAACCTGCCAG |
| | | | | | | 12 | |
| | | | | | 1409 | 20081 | CGGCGCCCAGCCCATCCGACTTTTGTAACACTCAGAATTGTAGTTTTGTT |
| | | | | | | 9113 | |
| | | | | | 1410 | 29254 | CGGGGCAGTTCAGCAACCCCGACCCCACCCGCGTGGCTCCAGGCCCAGGG |
| | | | | | | 680 | |
| | | | | | 1411 | 11328 | CAGAGGGGTGCGGTGGGGCCTAGGAGGCTCGTGGGCGATTCCGAGCACCG |
| | | | | | | 6610 | |
| | | | | | 1412 | 22369 | GAGATTAACGTCACCTCTCCTGCAAGAGCGGTTAAGAGGCAGAATCACCG |
| | | | | | | 485 | |
| | | | | | 1413 | 43276 | CCTTTGTCTCCCAAGTGGAGTGGTTCTAAATTTCTGATAACCAATGAGCG |
| | | | | | | 658 | |
| | | | | | 1414 | 15472 | CGCGGTCCTCCCGCAACACCTGCAGCCCCGCAAGCGAGCGCACTCGGAGC |
| | | | | | | 9059 | |
| | | | | | 1415 | 18546 | TTTCCGTGACAAACCCATTAGTTCGTGTGCAGATTTTTCCATTCAGGTCG |
| | | | | | | 2397 | |
| | | | | | 1416 | 16085 | CTGAGCGCACTCCTTCCACTGTACTGGGGGTGTACAGTGAGGAGTGGACG |
| | | | | | | 180 | |
| | | | | | 1417 | 46906 | GTTCTTAGTCTCCTGTCCCCAAACTTCTTAGAGTTATTCCTGTTGGACCG |
| | | | | | | 32 | |
| | | | | | 1418 | 14627 | CGCAGAAGAGCAGGCTATTGGAGCAATCCGAACCCTGCGCAGCAAAAGAA |
| | | | | | | 8678 | |
| | | | | | 1419 | 17702 | CGGGAATCCCGCACCACGGGGAACTCTCCACCCTGTCCCCGAAGTTTTGG |
| | | | | | | 4294 | |
| | | | | | 1420 | 37388 | GCAGTGGCCGGGGTCTTCTCTGGCCAGATCTGAGGGACCTCTGAGCTGCG |
| | | | | | | 828 | |
| | | | | | 1421 | 91194 | CGACCAAGACTTTTGCCCTTTTGAAATGAAATAGAAATAGGGGAGCTGCA |
| | | | | | | 708 | |
| | | | | | 1422 | 10031 | TCTGCGGCTCTGGCCGGGGGTCGGGGCTGTTATCTGCATGTGTGCGTGCG |
| | | | | | | 8321 | |
| | | | | | 1423 | 29902 | CGGAGTCTGCGAACGGAGTCTCCGCTGCCGCCGCTCAGCCCTCAGTCCTT |
| | | | | | | 2 | |
| | | | | | 1424 | 97171 | CGGCCCGCCCAACTGCTGCCGCCGCATCTGCAGAAACCACTCGTGTTGAG |
| | | | | | | 827 | |
| | | | | | 1425 | 60885 | GGTCTGCCCTTCCTCCCCTGCCGGGGCTACAGGGGTCGAGGGGCTGGGCG |
| | | | | | | 491 | |
| | | | | | 1426 | 13662 | AGGCTGCTCGGGGGTCAGACCTGGCGCTGGCGTTTCTTTCTGCTCTTCCG |
| | | | | | | 9816 | |
| | | | | | 1427 | 43741 | CGCACCCAAAATTGCCCCCAAAGGAGGGTTCAGTGCACGGCGCTCTGGAC |
| | | | | | | 310 | |
| | | | | | 1428 | 14748 | CGGGAGAAATTCTGAGCAGAGCCGTTAGCTGCTCTAACGTTAGAGCGTTA |
| | | | | | | 220 | |
| | | | | | 1429 | 10887 | CGCCTATCTGTTTGGAATTTCGCAAATAAGGAACGTTTAAATGACGTAGT |
| | | | | | | 003 | |
| | | | | | 1430 | 21123 | GCTGAACAACTTAACTCCTTGTCTCATAAAATTTAAGCTGCTATTGATCG |
| | | | | | | 51 | |
| | | | | | 1431 | 15847 | CGGCACCAGGGCTGGATTTATAGCAAGGAGCCCCGACTGTAAGAAGTAAT |
| | | | | | | 8629 | |
| | | | | | 1432 | 20084 | CGCTGCGCATCATCTTCGCCATCGAGAGCACGTTTGTGGGCTCCTGGCTG |
| | | | | | | 2890 | |
| | | | | | 1433 | 78756 | TCTAGGAAGCATCCCATTAGTAGCAATTAAGTCAGGCAAAAGACAGCACG |
| | | | | | | 422 | |
| | | | | | 1434 | 50203 | TGAGTACTCAGGTTGGCGTGGGACAGGGGAGCCGGCTCCCGGGGGCCACG |
| | | | | | | 4 | |
| | | | | | 1435 | 13472 | CACTCAGGCTGGCGTGGGGAGGGACACCCTTTGCATTCGTAAGGACACCG |
| | | | | | | 9942 | |
| | | | | | 1436 | 63504 | CGTGGATGTCTTCATCTGCCAGAGTCAAAGAAAAGGTCAGTGATACTATG |
| | | | | | | 673 | |
| | | | | | 1437 | 89952 | TTCTAAAATGTGGGCTTTCTGTGTACATGGGCGCGCATTCCCAGGACTCG |
| | | | | | | 052 | |
| | | | | | 1438 | 15811 | CGGCTGTGCTTATGTGCGTGTATGAGTTAGCAGCTGTGCACGTGTTTCTG |
| | | | | | | 4558 | |
| | | | | | 1439 | 21199 | CGGCTCCGCGCAGCAGCATCACCCGCACCACCTCGCTGTGGCCCATCTGG |
| | | | | | | 515 | |
| | | | | | 1440 | 68723 | GATTATTGAATAATTGCATAATTCCGAGCGCGGGTTGGGAGGCAATGCCG |
| | | | | | | 690 | |
| | | | | | 1441 | 13858 | CGGGTGCCCACCCTAGAATTAACCCTAGGATGGATGCAATCAGGATTCAG |
| | | | | | | 4567 | |
| | | | | | 1442 | 13735 | CGTAGCCAGCAGAGACCTCCGAGCACCGGCGCCCTCTAAAGCCGAGGTCA |
| | | | | | | 5128 | |
| | | | | | 1443 | 12726 | TTCTCCCAGTGCTTTGTTGACAAATGAACAAGATTTGACACTAACTAGCG |
| | | | | | | 5357 | |
| | | | | | 1444 | 11181 | CTACCTCCAGAGTGTGCCTTACTCCATCCAGGGACATGGATGAAGATCCG |
| | | | | | | 3852 | |
| | | | | | 1445 | 11372 | TAGGGAGGGCTTCTCACTTGGCTGTTACCTGGAAGTCCACAGTGGCCCCG |
| | | | | | | 7612 | |
| | | | | | 1446 | 13056 | CGGGGAACTCCAGAATTCGAATACCCCAGCCTTCTGTAGTTCTGGCCCCG |
| | | | | | | 6157 | |
| | | | | | 1447 | 24025 | CGGTGAAATGACTTGGACATACTTGTTGGTTTTCTGTGTTCTTACAAGAT |
| | | | | | | 7185 | |
| | | | | | 1448 | 55031 | ACCCTAAACCCTAAAAACCTCTGTACCTCCTGATATGGGTTGAATATTCG |
| | | | | | | 733 | |
| | | | | | 1449 | 15928 | TTGTACCTGCTGACTCTCTCTGGCAATGTGATTATCATGACCATTATTCG |
| | | | | | | 4298 | |
| | | | | | 1450 | 22424 | CGTGACAGGCCGAAGAAGTCAGGCTACAAACCGGATGACAACCGTAAGGA |
| | | | | | | 68 | |
| | | | | | 1451 | 60973 | CGCGGACCCCACCGCCTCTTCTGCGCGCTAAGGGTGAGCGCCAAGCAATT |
| | | | | | | 823 | |
| | | | | | 1452 | 24361 | TCACCATGGCCCAGAAGTAGGTGGCCATCTCGTGGCGGTTCTGCAGCACG |
| | | | | | | 81 | |
| | | | | | 1453 | 72301 | ATTTTAGTCCAACGGCCTGAGGAAACAGATACCCCTTGATCTGGAAAACG |
| | | | | | | 257 | |
| | | | | | 1454 | 10061 | CGGCTACCTCTACCACACAGTTGGGAAGCGCAGTCCTAAAGGAGACGCAG |
| | | | | | | 4879 | |
| | | | | | 1455 | 13425 | TTGCGGAAGGGCAGTGGCCAACTCTGCTTCCTTCTGGCTTCAGGCAATCG |
| | | | | | | 5144 | |
| | | | | | 1456 | 26835 | CGGCGCCTCACTCACTCCCAGAGCCAGAAACCTGTTCTCCCGGGAATGCC |
| | | | | | | 115 | |
| | | | | | 1457 | 17330 | TTCCAGGATTCAAGACGCTTCCTTTTCTCTTTTTTTGCTTTAAGCATCCG |
| | | | | | | 2602 | |
| | | | | | 1458 | 86088 | GGATGGCCGTCAGGGAATTAAATATGAATCTTTTGTCCTGTCCCACCACG |
| | | | | | | 946 | |
| | | | | | 1459 | 31739 | CGCGAGGGGGAGCAGTGCTCCCAACCCGCGACCCTCCGCCTCAGACATCA |
| | | | | | | 207 | |
| | | | | | 1460 | 24803 | CGGGTGAGAAAAGCTCAGGTGGGGCCCGCCGGGCCGAAGGAGGTAACCCG |
| | | | | | | 903 | |
| | | | | | 1461 | 11708 | CAAACAGAGGCAGGCTGCTTCCTGAGGAACCAGCCCCTCGGGGCTTGACG |
| | | | | | | 6931 | |
| | | | | | 1462 | 60921 | CGGACAAAGTGCAGCGACCACGGCCCCTCAGGTCCCGCCGCCTCCTCGAG |
| | | | | | | 891 | |
| | | | | | 1463 | 89188 | CGGCCACGACTCCAGTTACTATGATGACAGGTCAGACCATGAGCGAGAGA |
| | | | | | | 6 | |
| | | | | | 1464 | 11952 | CTCCAGGATGTAATTAGAGCTAAGAACAGCCGCCATCCCTCAGGGTTCCG |
| | | | | | | 2386 | |
| | | | | | 1465 | 13662 | CGGGCGGGACGCTCCTCTGGCGCTGTAGTGCTCCCAACTACCCTGCAGTT |
| | | | | | | 8604 | |
| | | | | | 1466 | 50513 | GCGGGTGTCCGTAACAGAGGGCGGGCAGAGTCGGGGGTGCGGGGCCTGCG |
| | | | | | | 870 | |
| | | | | | 1467 | 96623 | CGCGGTCTCTTGAGAGCCCTGCAGAGCCCCAGGCACTTTCCGCGGGACCA |
| | | | | | | 674 | |
| | | | | | 1468 | 85672 | TTGGCCGGGCGAGCAAGGCTGCTGCTGCCAGCTGATCTGCTCGGGATCCG |
| | | | | | | 285 | |
| | | | | | 1469 | 31739 | TCTGAGGCGGAGGGTCGCGGGTTGGGAGCACTGCTCCCCCTCGCGGAGCG |
| | | | | | | 202 | |
| | | | | | 1470 | 47483 | TGGCTACTTGTCCTGGCAGGCACTTCTTGATTGGCCGGCGTGCTTGTTCG |
| | | | | | | 268 | |
| | | | | | 1471 | 47483 | GAGGCTCTAAGGGGGCTGCGCAGGGCCCAGACATTGGGGTTATGGCAGCG |
| | | | | | | 418 | |
| | | | | | 1472 | 13545 | TACTGACAACCCGCTAATAGTCTTGGGCTCCAAGAAGCCCGGCAAAACCG |
| | | | | | | 5174 | |
| | | | | | 1473 | 28218 | GGAGACCAGTTCGAGCACTAACGCGGGGGCGCGCGAGTGTGAGGGTTGCG |
| | | | | | | 959 | |
| | | | | | 1474 | 13968 | CCACAGTTGCAGGCTCTGTGGTCAGCTACGGGGTGACGAGAGTGGAGTCG |
| | | | | | | 6745 | |
| | | | | | 1475 | 37390 | ATTGGGGTGCGGGGCGTGCGCGCCGTAGTGCTGTGTGGCGTGGAGCGGCG |
| | | | | | | 284 | |
| | | | | | 1476 | 21736 | CGCGCCCGGCCCCACAAATGTAAGATTTATGCACTTCTCCAAGCCAAGTG |
| | | | | | | 3253 | |
| | | | | | 1477 | 43185 | CGGCCCCATCACTCAATCAGCCGAGCATTCGAAACCAGGTCTGCCAGGCA |
| | | | | | | 803 | |
| | | | | | 1478 | 12644 | CGTGAGCCCTGAGATCCTCAACACCACTGGGACCTACTCCGGAAAGGCTG |
| | | | | | | 8338 | |
| | | | | | 1479 | 14348 | CGGCACGATTCTCTAAGTTCGGAAACCACGAAGCTTGCGCCTTCATGCGT |
| | | | | | | 844 | |
| | | | | | 1480 | 61616 | AGTGAATGGGGAGGCGGGCGGAGTCGGGCCCAGCCAGGCACCACCTAGCG |
| | | | | | | 492 | |
| | | | | | 1481 | 20145 | CGCCGAGTGGGGCGGGGCTTGCTGGAGCCTAAAGTCCAACCCATCCACCC |
| | | | | | | 0743 | |
| | | | | | 1482 | 13546 | AGCGGCGGGGGCGGCTCGCTGGCGCCCTGGAGTCCGTGGATGAGGATGCG |
| | | | | | | 4814 | |
| | | | | | 1483 | 53083 | ACTCCCGAAGCTGAATTGATTTTCAAATCTGGAGGCGTCTCTCGGGGACG |
| | | | | | | 532 | |
| | | | | | 1484 | 13060 | CGCAAGTGGAAAACTATGCGCTGTCCGATCGCAACCCTCCAATCTGTCCC |
| | | | | | | 915 | |
| | | | | | 1485 | 17519 | CTCCAGCTCTGTTGTAATTTTGTCTTCGCCAGATAGATTAGCTGGAACCG |
| | | | | | | 3377 | |
| | | | | | 1486 | 49378 | CGGGAGCAGACTCAGCTGTCCCCAAACCTGAATGCAGGTAACAAGGGCAG |
| | | | | | | 497 | |
| | | | | | 1487 | 15414 | AAGGTCCGGTGGCCGGAGCGTGCGGGAAGCCTGCCCAGAGTCCATTTCCG |
| | | | | | | 4286 | |
| | | | | | 1488 | 13205 | CGCGGCTCATCTGTCATTAGCACCGGCACTAAGCTCCCACCGCTCAGCGA |
| | | | | | | 4555 | |
| | | | | | 1489 | 35078 | GGCTCTGAGGCTGGACCCGGGAACTTCTGTGGCTGGGAGTGTTGCACTCG |
| | | | | | | 9 | |
| | | | | | 1490 | 14079 | GAGAAAAGTCAAGTTGCAGTCCCACACAGAGCCTCTGGGCGCCGCCGTCG |
| | | | | | | 7172 | |
| | | | | | 1491 | 50891 | GAAATCTCACGGTGACCTGGCCTCAAGCGAGAAGGATACCTGGGCCACCG |
| | | | | | | 341 | |
| | | | | | 1492 | 17974 | CGCGAGAGATGGAGCTGCCCACGGTGTTGGTGACGCCCACGGTGAGAGCG |
| | | | | | | 0914 | |
| | | | | | 1493 | 98221 | CTCCATTCGTAGCTTTAATGCTAAGACGTTCATTAGGCGTTGCTCTCACG |
| | | | | | | 619 | |
| | | | | | 1494 | 97646 | CGCCTCGCCCACTTCCCAGTAGACACAGGGACAGCGGCTTCCACTCCCGG |
| | | | | | | 731 | |
| | | | | | 1495 | 15130 | CGGGGCTCTTCCCAAGCCACAAGTAGACTTGGCAAACTTTTTGTCCTAAA |
| | | | | | | 7730 | |
| | | | | | 1496 | 31739 | CATGAGCTTGGGTCTCTCGGAGGAGCAGGAGACGCGGGCTCGAAGCCGCG |
| | | | | | | 423 | |
| | | | | | 1497 | 10547 | CGGCCAGGTCATAAACCCGTCAGGCCGCCGGGCTCCGGATTTGCAGCCTG |
| | | | | | | 9318 | |
| | | | | | 1498 | 10428 | CGCCGAAAATTTATTGACGCGGCGAGGCCAGGCTAACAACACTGCAATCC |
| | | | | | | 69 | |
| | | | | | 1499 | 23820 | TTTGGAGACTTAACACCGGCCGCGTTGTGTGCGGAGGTGGTGTAGGGCCG |
| | | | | | | 979 | |
| | | | | | 1500 | 61655 | CGCCGGTGTTTGGGAATCCCAGCGGGGTCCCCAGCTTGCCGATCTCCCAG |
| | | | | | | 919 | |
| | | | | | 1501 | 19483 | CGCCTGGGGACACCGCTCAAATCCTAACCCTTGGCCGCCCGTCGGCTCCT |
| | | | | | | 721 | |
| | | | | | 1502 | 14993 | CCCAGGTCGCTGTTTCGCGCTCTGTAGGGGATTGGACCAGGAAAGTAGCG |
| | | | | | | 584 | |
| | | | | | 1503 | 64549 | AGTCGGTGGGCCAGTGCAGTTATCCTAGCAAGAGCGGGCAGAGGATGCCG |
| | | | | | | 818 | |
| | | | | | 1504 | 26273 | GCTTTTACCCAGGTAACTAGTTTACTTGGAGCTGGTGTACTTGGTGACCG |
| | | | | | | 556 | |
| | | | | | 1505 | 10091 | CGAGCGGAGGACGCTGTGTGTGCATAAATTTCAGCAACGCTGAATACAGG |
| | | | | | | 6959 | |
| | | | | | 1506 | 22094 | CGAGTTTCTAAATTGCACAGACTGCTCTGGGCCGGGGCAGACACCGCGGC |
| | | | | | | 226 | |
| | | | | | 1507 | 23412 | CGCTACCGCTACCGCTTCTGCTCTCGGTTATGGCAAAAGAGCCACCATCA |
| | | | | | | 409 | |
| | | | | | 1508 | 13094 | GGCGTCGCGGATGCGCCTCAAATTCAGTGCAAAAACGTGCTGAAGATCCG |
| | | | | | | 77 | |
| | | | | | 1509 | 84820 | GTGGGATGTGTGAGCCGGATGCCCTCTCCTTCCGGTGTGGCGAGGACGCG |
| | | | | | | 99 | |
| | | | | | 1510 | 61149 | CCCCGGGCACAGGAGCCAGCTCGGCTCACGGTGGGGTCTGCTTGGAGGCG |
| | | | | | | 009 | |
| | | | | | 1511 | 10460 | CGACCTTGAAGAAGATACACGGGCTATGACTTCGCTGTTGTCACCGAGCG |
| | | | | | | 9432 | |
| | | | | | 1512 | 18771 | CCAACTGGGGGCGGTAGGGGCGGTAGTTACTCGGAAATCCATGTATTCCG |
| | | | | | | 3711 | |
| | | | | | 1513 | 13991 | CGCAAAACTTGTCGCTGCCCAATAGCACGGCCCAAGCACTCTTGGCCGCC |
| | | | | | | 6869 | |
| | | | | | 1514 | 89952 | CGCTTTCATTTTCAGAGCTGCCTGTGGCCTTACGCAGGTTCAGAAAATCG |
| | | | | | | 135 | |
| | | | | | 1515 | 92947 | CGCGGCTTGGTCTTCACAGGCGCAGATTGGGGGCCTGTTTCATTTTTCGT |
| | | | | | | 961 | |
| | | | | | 1516 | 10426 | CCGCTGGAGAAGCGGGCGCGACAGGGCGATGAGTTAACGCGGAGGGAGC |
| | | | | | | 05 | G |
| | | | | | 1517 | 12605 | CGTGCCTACAAGAATTCAGACGTTAGGGAATTCAAGACCATGCCTTAGAC |
| | | | | | | 8 | |
| | | | | | 1518 | 99276 | CGGCCGAGTCATAGGCCTGATCATATTAACGCCCTAGGTCAGAGGGAGAG |
| | | | | | | 357 | |
| | | | | | 1519 | 11537 | CGCCTTCAGCTCCCACCCGCAGCAGCAACCGCAGCCTGCTTCCGGGGACT |
| | | | | | | 7744 | |
| | | | | | 1520 | 39466 | CGGTTGGTAAACCGCTTAGCTGTGAGCCCGACCCAGGGCCTGATTCGAAA |
| | | | | | | 757 | |
| | | | | | 1521 | 20898 | ATGCTCCTGAGCGGGATGAGGGAATCCCAGGGCACAGAGGTATCTCTGCG |
| | | | | | | 785 | |
| | | | | | 1522 | 43276 | CTGTCCAGGTGTCTTCGTTACACATGTGCCTTCCTAGTCTGGAGTCCACG |
| | | | | | | 781 | |
| | | | | | 1523 | 98356 | TGTCTCCACACACAGCTGGGCTGTGGTAGGGGGTGTCTCAGGCCACACCG |
| | | | | | | 101 | |
| | | | | | 1524 | 13611 | GGCTCCAACACGGAGTGCGCGTGCGCTCAAGGACCAGCTGGCTAGGAGCG |
| | | | | | | 4331 | |
| | | | | | 1525 | 10882 | CGCGGCCAAGGACAAGGCCCCGCCCGGGTCCATAACCATTGTCCCCAGCT |
| | | | | | | 240 | |
| | | | | | 1526 | 90097 | GAATCTGCCCCTTAGCCCGTATTGCAGGGGCGGTGCAGTTGGGGATTCCG |
| | | | | | | 981 | |
| | | | | | 1527 | 62360 | CGCAGGTGGAATGGTCTGCAAACAGATGAATTCAGCCCCTCCAAACCAAG |
| | | | | | | 417 | |
| | | | | | 1528 | 21084 | TGCAAGATGAGTCGATCAGTGACGCGTGGGGTGGCATTGTTCGCTCGACG |
| | | | | | | 420 | |
| | | | | | 1529 | 26271 | CGCGCAACAAAACCATTGGCTGAAGAGTCCAACGATGTGATTGGTGAATT |
| | | | | | | 716 | |
| | | | | | 1530 | 51416 | CGCCGGCTCCGCGGTCTACACCCACCCACTCCTTGGATGATCTCACCCGG |
| | | | | | | 213 | |
| | | | | | 1531 | 29937 | CGCTCAGAATTTGAGTGACAGAACTTGCCACCACACAGTGCACTTAATGA |
| | | | | | | 0 | |
| | | | | | 1532 | 13168 | AATAAGGCCTCGGAACCCACCAGTTCAGCCCTGGGCGGGAGTCGGCAACG |
| | | | | | | 4923 | |
| | | | | | 1533 | 13481 | TATGGGAGGTGGTCTCTGGCAGTACTCTCTGAGTGCTGCCTCCCGAGACG |
| | | | | | | 8 | |
| | | | | | 1534 | 60705 | GGCGTTGATATTTTCTTTTCCACACTTAGTCTTCAAGGAGGCTTGTGGCG |
| | | | | | | 839 | |
| | | | | | 1535 | 77585 | CGGGAGGGGGACGCAGCCTAAAGACCTCGTACTCTTGCTTCTTGTTTCTA |
| | | | | | | 318 | |
| | | | | | 1536 | 13356 | AGTGGCCTGCACAGGGGAACTGGGTAGCGAGAGAGTTCCAGGCAATTCCG |
| | | | | | | 2056 | |
| | | | | | 1537 | 13988 | CGCACAGCGCCACAGGCATTCAGGGTGTTTATTGCGCTTGGGGCCAGGGT |
| | | | | | | 0223 | |
| | | | | | 1538 | 81189 | CCACCGGCGGTCTGAAGATTTGGCAGCCAGCAGTCTCCAGAGGGGGGTCG |
| | | | | | | 706 | |
| | | | | | 1539 | 13358 | GTAATAATAGGTACCCCTTTCCAGAAGAGGGTTCAAGATCTTCCAGAACG |
| | | | | | | 9654 | |
| | | | | | 1540 | 40120 | TGGCACGGGTCATCGTGGACAAGTACCGTGATGGCACCAAGATGGTGTCG |
| | | | | | | 327 | |
| | | | | | 1541 | 33700 | CGCGGGCTTAGCAGACCCGCCCATTGGCTCTTGCATCTCCTGCGTGGGAT |
| | | | | | | 747 | |
| | | | | | 1542 | 23412 | CGGCTAAGTGCCCCTCTCGTATCAGGTGGCCGGACACATGAACGGGAAGG |
| | | | | | | 250 | |
| | | | | | 1543 | 37123 | CGCGGCCTCTAGAATATTGTAGAATCTTCCCAAGAGCCCCCGCGGGAAGG |
| | | | | | | 669 | |
| | | | | | 1544 | 10157 | CGCAGTGCCTCCGCGAAACTGACAACTCCTCCGGGCGGTGACCGGCTCCA |
| | | | | | | 0703 | |
| | | | | | 1545 | 57283 | GCCAGGGCCGGCTGTGGTCAGGCGGGCTATTTTCTGGCAAGTGTGGCGCG |
| | | | | | | 436 | |
| | | | | | 1546 | 23563 | CGCCGCCGCCACCAGCGTTGTGAACCTCTGACCCTCGCGGCTCTGCGTCC |
| | | | | | | 925 | |
| | | | | | 1547 | 79627 | CGACCCGCTAGCCTAGGCCGAGACACAGTCTGTATTCTGAAAGCCGCCAG |
| | | | | | | 216 | |
| | | | | | 1548 | 24203 | CGCGCCGTTGTCTAAAAAAGTCTGAACTCCACCTTAGTGTTAACTAGTCC |
| | | | | | | 1694 | |
| | | | | | 1549 | 17702 | GTCACGGCCGCTGACAAATAGTTCCCTTTGCTTCCTGATTTGGAACTGCG |
| | | | | | | 2285 | |
| | | | | | 1550 | 10834 | CGGATGAAGCCCAAGGAACCACAGGGAGATAATTTGGTTTGGACAGGGCT |
| | | | | | | 29 | |
| | | | | | 1551 | 65594 | CGTTAACACTGAGCCATGAAGCCAGGGCTTGTGTCCTCAGAGAATGCATA |
| | | | | | | 890 | |
| | | | | | 1552 | 52626 | CGCCCCGCCGAAGCCCAGGCTTTCAGTCCAAGCAGGGATGGTCCGGAGTA |
| | | | | | | 889 | |
| | | | | | 1553 | 96674 | TAAATAAATGTCAAACACACACAAAATCCACTCTGAGGCGGGTGCTGACG |
| | | | | | | 333 | |
| | | | | | 1554 | 11207 | CCTTGCTTGCTGGGGACTGGGGCCGCGAGGGCATACCCCCGAGGGGTACG |
| | | | | | | 3373 | |
| | | | | | 1555 | 25034 | GGCCTGTGGGCGCGGCGCAAAGCCCGGACTGGATTCCAGAGGGGCAGGCG |
| | | | | | | 220 | |
| | | | | | 1556 | 26226 | AGAAACCTACAAAAAATTAAATTTGCCCTGTAAAACACTAGTCTGAATCG |
| | | | | | | 964 | |
| | | | | | 1557 | 27258 | AATCTTGGAAAACAGAAACACCCCGAAAAATTGCAAGTTGTTTCCGCTCG |
| | | | | | | 466 | |
| | | | | | 1558 | 22094 | AAACAATGCTTATCTCAAGCTGACTCAAAACTGGCTCTTGAGAAAATCCG |
| | | | | | | 963 | |
| | | | | | 1559 | 14937 | CGCGCACCTCCGCCAGCAGTCCTACGACGTGACCGACGAGCTGCCACTGC |
| | | | | | | 4763 | |
| | | | | | 1560 | 61616 | CGGGAGTTTTCAGTCAAGAAGCGATAATGCGCTTTGGCTATTTGGGGTTT |
| | | | | | | 390 | |
| | | | | | 1561 | 35046 | ATGCACAGCGATGGGGGAATGGACCATCTTGGAGAGGCTGCTAGAAGCCG |
| | | | | | | 650 | |
| | | | | | 1562 | 10090 | TGTCTGGGTTAATGTTTTATAAGGGACTGGCTACTCTCGTCTACCTGCCG |
| | | | | | | 4055 | |
| | | | | | 1563 | 32484 | GCAGCGCGCGGCGCGGAGAGGCGAGGACCGGTATCTGGAGACTGGGGAC |
| | | | | | | 259 | G |
| | | | | | 1564 | 32110 | AGGGGCGCGAAGGCAGGCGCGGCCAGGGAGTGAGCGGGGCGCTGGGCGC |
| | | | | | | 523 | G |
| | | | | | 1565 | 10091 | GATCGCATTCTGGGTGCCTGAGGGCCAGAGGCTAGGTTTTAAGGCCACCG |
| | | | | | | 5395 | |
| | | | | | 1566 | 87138 | CCGGGCAGGAGATCTGGGCTGCGGGCGCCAGGCAGAGCTGGCATCTTCCG |
| | | | | | | 203 | |
| | | | | | 1567 | 61616 | TAATTTGTATGGAACCACGAAAAACCCCAAATAGCCAAAGCGCATTATCG |
| | | | | | | 369 | |
| | | | | | 1568 | 24648 | GGGGCTTGGCACACAGTAGGTCTAAGGCACCGAGGCAGGAGCGTCCCCCG |
| | | | | | | 696 | |
| | | | | | 1569 | 38964 | CGCTGACTGTGAATAAGTGTGTCAGTTCTTAAGGTCCAGCAACACAAAGC |
| | | | | | | 944 | |
| | | | | | 1570 | 11581 | GGTCAGTCCTGTAGTGCGTAACGGAACTCGGTTCCGGCTGGCTCTGATCG |
| | | | | | | 9158 | |
| | | | | | 1571 | 65331 | CGCCCAATGTTTTTGCAAACTCTTGCAGCTGGGGGAAGGCAGTGGATACT |
| | | | | | | 63 | |
| | | | | | 1572 | 19384 | CGGTTGGGTTTATAGAGTTGTGATCACCAAAGCCTGCATTTAAAATAGAC |
| | | | | | | 8133 | |
| | | | | | 1573 | 19577 | AACGGCTTAAGTCAATTATAACCTTCCTTTCAGAACCATCTTGTGGGACG |
| | | | | | | 822 | |
| | | | | | 1574 | 10847 | TGAGGGGACAATAGCTGGTCTTACAAAGGCACCACCTGTGATTTAGCACG |
| | | | | | | 9584 | |
| | | | | | 1575 | 18489 | ATAACCGTCTCTCTCTCAAAACGTTCTTTATTAAAAGGCACTGGCGGCCG |
| | | | | | | 660 | |
| | | | | | 1576 | 13546 | CGCCCCAGCCCAGTGCCCCCCGAAGGGCCAAATGCCAAGTCCACTGAGGC |
| | | | | | | 5030 | |
| | | | | | 1577 | 14492 | GGAGCCGCCCGTTTGCCTGGAGAGCAGCAAGGACAAAGCAATGGGCTCCG |
| | | | | | | 02 | |
| | | | | | 1578 | 91192 | CCATGCCATGAGAGAGAACCTGGCAGTGTCAGATTCACTCCAGCGGGTCG |
| | | | | | | 798 | |
| | | | | | 1579 | 17703 | CTCAGAGAATACCTTCCTTCCGGCAGGAGACCGTTTGGCCCTGTATTCCG |
| | | | | | | 0228 | |
| | | | | | 1580 | 15112 | CGAGACTAAGAAGTGACCCAACCTAGGTGACCCTTCAGCTAGGTCACTCT |
| | | | | | | 9081 | |
| | | | | | 1581 | 26273 | CGGTTTCTCATTGGTTCTTTTAGTGTCTGAATATATGATAGGCCATTTCT |
| | | | | | | 096 | |
| | | | | | 1582 | 98982 | GTCCCTTCCCTGGCTCAGGGCTGGCTGGAAAGACAGAGACGTTTCATGCG |
| | | | | | | 28 | |
| | | | | | 1583 | 34392 | CGCGGTCCTGGCGCCCCCACACCCGCGACCCCGCGAGGCCCCCACCTCCT |
| | | | | | | 7 | |
| | | | | | 1584 | 10089 | AGATTCGCTTTTCTAACTACGGAAGTAAGGCTCTGGGCAGCTGAGCTCCG |
| | | | | | | 5618 | |
| | | | | | 1585 | 38080 | CGCTGGAGACAAGCCCCAGCAGAGCGAAATATCCTGAAGTAGCTTTCTTT |
| | | | | | | 303 | |
| | | | | | 1586 | 32096 | CTAGGGCAGCCTCAGTTGGCAGCCCAGAGCTGTGACCGGGTGTTGGAGCG |
| | | | | | | 716 | |
| | | | | | 1587 | 18771 | CGCCCTTGACTGAGGAGTGTTTTTAATAATCATCTGGCCGACTTGGAAGC |
| | | | | | | 4068 | |
| | | | | | 1588 | 10642 | CGCTCCCACCGCTACGCGGGGCCGCCTGGCTCCTGGTTCTCATCGTTCTT |
| | | | | | | 9316 | |
| | | | | | 1589 | 26251 | TGTATTCTTACTCCTTTATCTTGTTGCAATGCCTGATCCAGCTAAGTCCG |
| | | | | | | 899 | |
| | | | | | 1590 | 16369 | CGCGCGAGCCGCTCTTTGTGGCAGAGCATCCTCTTTTGAAACCAGAATTC |
| | | | | | | 5111 | |
| | | | | | 1591 | 26250 | AACTGTTTGTTCAGTTGTGACCATTGCTTGAAACCCATTCCTATGGCACG |
| | | | | | | 826 | |
| | | | | | 1592 | 18891 | GGGTTTGCGTGCGGGCCGGGTGCCTGGGGGCCCCGGGCTGAGGGTGAGCG |
| | | | | | | 6724 | |
| | | | | | 1593 | 74928 | TTGGAGATCTCGGAGTTGTGCATCTTGGGGTTCTGCTGCGCCATCTGGCG |
| | | | | | | 01 | |
| | | | | | 1594 | 13611 | CGCTGCAAATAACCCCCACCACAAGCACTGCCGGGAGCCCAGAATAGCCT |
| | | | | | | 370 | |
| | | | | | 1595 | 40120 | CGGCCATTTCAAAGAGCTGCTCCAGCCGTTCCCGTTCGTGGTTGGTGTCA |
| | | | | | | 496 | |
| | | | | | 1596 | 81897 | TCACCACACAATTGGGCTTTTTACCTCTCTTTAAACAGAATGAAATGCCG |
| | | | | | | 627 | |
| | | | | | 1597 | 13611 | CGGATGCCCCCAGGCTGTGTGAAGAAAATTCAGGGCAGGCTGCGAGTCTG |
| | | | | | | 481 | |
| | | | | | 1598 | 57037 | TTAATAACATACCAATTTTGCCACCCTTGGCATAGGGAGCTAGCAGATCG |
| | | | | | | 657 | |
| | | | | | 1599 | 44185 | CGGGCGACCGCAAGCAGATCTACAACATCCTGAGCACGCTGGGGCTGCGA |
| | | | | | | 323 | |
| | | | | | 1600 | 86383 | CGGTGCTCAGTGTTGCCTTGTGCTGTCCAATATAATAGCCATTCGCCACG |
| | | | | | | 761 | |
| | | | | | 1601 | 12721 | CGGGCTCCAGGACCGTCCCCTGTGCGCCCTCATCTGCACCGTCGCCGTCC |
| | | | | | | 1262 | |
| | | | | | 1602 | 60985 | GAAGTCGTCGCCGGCCTGGCGGAGGGTCAGGTGGACCACAGGTGGCACCG |
| | | | | | | 636 | |
| | | | | | 1603 | 21023 | TCTTGGAGGGTGTCGGGGTTTTCCTTTCTTTTTCATTTGAATTTTATCCG |
| | | | | | | 042 | |
| | | | | | 1604 | 32223 | AGCCCTGGAGGAATTTCCCTTTTGAAATCTTGCGGTGTTGAGTAGGACCG |
| | | | | | | 098 | |
| | | | | | 1605 | 62583 | CGGCGTTCCAGCTCTCAACCTTGCTCCCAGCAGCCCCCGCCGGCGCACTC |
| | | | | | | 919 | |
| | | | | | 1606 | 10843 | TCTAATGAGAGATGACGGCCAGGTGTTCTCAGCAGCCCCTGGCCCTCACG |
| | | | | | | 6768 | |
| | | | | | 1607 | 95240 | CGGAGATGTCCTCCAGAAAGCAGCAGAGCCATGGAACGGGGTTTGAGCAC |
| | | | | | | 444 | |
| | | | | | 1608 | 10506 | TGGAGTTTGGTGCGGTCCCGGTGCCCAGGGGGCGCCTTTTCCGCATGCCG |
| | | | | | | 7038 | |
| | | | | | 1609 | 99178 | GCATTACAGGGTTCCTGGTGTGTTCCTCTAACCACTTAGTTGGGAAATCG |
| | | | | | | 39 | |
| | | | | | 1610 | 63614 | CGCGAATGGCGGTTACAAGTGCAGCTAGTATAAAAAGCTGAGATAAGGGT |
| | | | | | | 857 | |
| | | | | | 1611 | 20212 | TAAATGCTTTCCAATAAAGCATGTCCAGCGCTCGGGCTTTAGTTTGCACG |
| | | | | | | 2669 | |
| | | | | | 1612 | 99986 | CGGGTCACTCAACTAAGAGTAGCGTCCAAAGGCAGGATGCGGTAACACCT |
| | | | | | | 964 | |
| | | | | | 1613 | 10531 | TCAGAGGAGTATCAGAGCCAGCGCTGTGTGTGCAGGGCTGGGCCTTTCCG |
| | | | | | | 9819 | |
| | | | | | 1614 | 85673 | CGGCCTCGTAAAGCTCCCAGCTGGCCCGGACCCCGAGCTCCTCCCGGTTA |
| | | | | | | 270 | |
| | | | | | 1615 | 22040 | AAATCTAAGTAAGCTGGTCCACTGATGAAAAACCTCCTTCCTTGCATGCG |
| | | | | | | 307 | |
| | | | | | 1616 | 32322 | CGCCGCGGAACTCACAGAGGTTCCCCCGCCATAGCCTCCCGCGTGCGCTC |
| | | | | | | 46 | |
| | | | | | 1617 | 73976 | TCCAGTCCCGAGCATTTCGGATGAATGAATGATGAACAAACGACAGAACG |
| | | | | | | 790 | |
| | | | | | 1618 | 41363 | CGCAGCAGCCTGAGACGCTGCCAGCTCTTCAAACGTTACCCGAGGGCCCG |
| | | | | | | 789 | |
| | | | | | 1619 | 11219 | CTGTCGGGTGCAGGTGGACGTCCCCTCACGGTAAGGTCAGAGTGCTCCCG |
| | | | | | | 29 | |
| | | | | | 1620 | 63005 | GCTTGCGAAGTCCGGCTTCTCTGAGGCCCCGTAGCTGGCGCGAGTTGTCG |
| | | | | | | 077 | |
| | | | | | 1621 | 14937 | GGGCAGTGGCAGCTCGTCGGTCACGTCGTAGGACTGCTGGCGGAGGTGCG |
| | | | | | | 4761 | |
| | | | | | 1622 | 22317 | CGCCCTGCGGGGAGTCGCCTTCGTTTAGTCCCAGGTTTCTCCGGCGCTTT |
| | | | | | | 6606 | |
| | | | | | 1623 | 38821 | GGAGCCTCGCAGAGTTTTAAAGATGCAGAGCTCCTTTTTCGGAGGCCACG |
| | | | | | | 487 | |
| | | | | | 1624 | 62305 | AAGTGCTGATAGTGGGCCTCACAGAAAAGTTACCATGAACCGACTTTCCG |
| | | | | | | 003 | |
| | | | | | 1625 | 13974 | GAGAGAACGGAGGTGCGGATGCGGCGCAAGCAGAGGCGCAGGCGAAGGC |
| | | | | | | 467 | G |
| | | | | | 1626 | 10280 | AAGTCCTTCATCACCAGGAGTGTCTGCTTAAGTCTATTCCAAGTTGAACG |
| | | | | | | 3290 | |
| | | | | | 1627 | 26252 | CGAGGGCACTAAGGCCGTCACCAAGTACACCAGCTCCAAATAAATGGACG |
| | | | | | | 265 | |
| | | | | | 1628 | 53446 | CGTGAAGTGGGTGCTTCAGCTGTCCCCCAGCTTTAGGATTTTAACCCAGC |
| | | | | | | 784 | |
| | | | | | 1629 | 18005 | CGCGGAGTGGACGCAGCCACGTCTGCAACTCACCCGGACACACACAAAAA |
| | | | | | | 42 | |
| | | | | | 1630 | 69451 | ATGCGCTCCGGAACCGTAATGCGCGCTTTTTCTAAGCCTTACGGTAAACG |
| | | | | | | 412 | |
| | | | | | 1631 | 52858 | GCACATGCGGATGCGGAGAGAGGCCCATGGCGAGTTCGGCGGCGTGGCCG |
| | | | | | | 0 | |
| | | | | | 1632 | 21635 | CGGGGCCTCCCAGGCCACTCCTTCTAGGACAAGTAGGGTCTGGGCACTGT |
| | | | | | | 29 | |
| | | | | | 1633 | 56932 | GCGAACCAGGTTGGGCGGGGCCTGGGCGGGGCTCTCAGATAGCGAAGGCG |
| | | | | | | 872 | |
| | | | | | 1634 | 77184 | CTGTCCAAGCCTGGAAGGGGTCATTTATTCCATGCAGAAGGAACTGGGCG |
| | | | | | | 194 | |
| | | | | | 1635 | 15503 | CGCCTCAGACGAGGGCGGGCTGTGTGGGGCGGGAGTACCAGAAAGGGTCG |
| | | | | | | 5375 | |
| | | | | | 1636 | 76923 | CGGGACCTCCCAAGTAAGGGACAAACGCGCTTCTCCAGAAGAGGCGTGTC |
| | | | | | | 943 | |
| | | | | | 1637 | 37571 | CGCGCAGGCTGCAGCCCTCCTACCCCGGACCTTCGCAAACCCCGGGGCTG |
| | | | | | | 729 | |
| | | | | | 1638 | 36604 | CGCATGGAATCTGGTGTTTATTACACTCGGGGGAGAGAGGAGGTCACTCG |
| | | | | | | 614 | |
| | | | | | 1639 | 39466 | GCAGCGGCGGGCTAAGCTTCCCTTTCCCACTTTGTTCTGGGAGGCAGTCG |
| | | | | | | 295 | |
| | | | | | 1640 | 10291 | AGCTGGGGGCTCTGCGAGGGGGCGGGGCTCAGACACGGGTGGGGTCACCG |
| | | | | | | 88 | |
| | | | | | 1641 | 51231 | CGCAGTAACCGGATGACTCCTACGGGTCACCTCGCAAGCCCCGCCCCCTC |
| | | | | | | 736 | |
| | | | | | 1642 | 35479 | CGCGAGGCAGCGCCTCTACCCGCTACCCCCAAGCCTGGGCGCACCCCCTC |
| | | | | | | 628 | |
| | | | | | 1643 | 44923 | CGAACCTCAGTTGAGAAACAACAGAAGCTGAGACAATTTTACGCAGGTGA |
| | | | | | | 719 | |
| | | | | | 1644 | 41797 | CGCCAGCATCCGTCAGGAGGATGCCTTTGATAACAAAATTGACATTGCTG |
| | | | | | | 729 | |
| | | | | | 1645 | 11513 | AACTGGAAGCTATGTCGCCCCTTCTTTCAGGGCCCAATTAATTCGCCGCG |
| | | | | | | 6096 | |
| | | | | | 1646 | 80976 | CTGCGCCGCGTCCATGTAGGAGTGGCTGAGGCCCGGGTGGTGCGTGTCCG |
| | | | | | | 89 | |
| | | | | | 1647 | 84821 | CGCCACACCGGAAGGAGAGGGCATCCGGCTCACACATCCCACCCTATGTC |
| | | | | | | 07 | |
| | | | | | 1648 | 35298 | GGCCTGGGTGCCCAAAGTGGCAGTGTGGGCCTCTGTGGGATGGAGAGGCG |
| | | | | | | 199 | |
| | | | | | 1649 | 10914 | ACTGCTTTTCTGTAAATGACAGAAATACTAAGAGAACTTGACGGAAATCG |
| | | | | | | 9011 | |
| | | | | | 1650 | 16225 | GTCTGCATGTGTCTCCTTAGCCTTAACCCCCTTGCCTAGGACAGAAAGCG |
| | | | | | | 9067 | |
| | | | | | 1651 | 62620 | CTGAACCACCAGTGATGAATTGCTGCTCACCAGTGATGAGTTGAATACCG |
| | | | | | | 843 | |
| | | | | | 1652 | 78172 | GCACGTCGGGAACCCGCGAGTTAATAGAGGTGACCGAGGACGTGGACCCG |
| | | | | | | 337 | |
| | | | | | 1653 | 29186 | GCTGACTTGATGCGGCCAGGTTTCTGGGACCAGGAGGTTGGGACACGACG |
| | | | | | | 231 | |
| | | | | | 1654 | 27282 | CGGACACCGAGTCGGATTTCTATGAAGAAATCGAGGTGAGCTGCACCCCG |
| | | | | | | 987 | |
| | | | | | 1655 | 12898 | AGCAGCAGCGTGGCCAGCACGTAGCCCAGCTGCTGGAAGAAGTGGGCTCG |
| | | | | | | 35 | |
| | | | | | 1656 | 26250 | AGCTTGCGAATCAGTAGCTCAGTCGATTTCTGATAGCGGCGGATTTCACG |
| | | | | | | 685 | |
| | | | | | 1657 | 10421 | CGGGCCTCTAAAACATGAGCTCTGCCTTCTGGGATCAGTGCACATAGACC |
| | | | | | | 6442 | |
| | | | | | 1658 | 66275 | GCGGGCTGGCCTTTCTGCTGCTGGTAGGAGGATCATGTGCTGCTATTTCG |
| | | | | | | 988 | |
| | | | | | 1659 | 32016 | CGCTTGTTTTAGAATCTGTGCCCTGCATTGCTGTAAGCAGCTCACAAACA |
| | | | | | | 535 | |
| | | | | | 1660 | 10799 | CGGGCAGAGGAACAGGAGGAGAAACTGCCGACAATCCCTCATTTCCAATG |
| | | | | | | 5 | |
| | | | | | 1661 | 75316 | CGCCGCTGCTAAATATATCCGTAGGAATGGAGAGGGACCGGATCTCAGGT |
| | | | | | | 383 | |
| | | | | | 1662 | 50683 | GGGTGCATTCCATACTGAGAGCCATGGCTCATGAGGGACAGGTCATGGCG |
| | | | | | | 213 | |
| | | | | | 1663 | 17275 | CGGATTATTGCCTCTAAAAGGCTGGCCTGGCACCCTGCCTGGCAGCATCT |
| | | | | | | 1061 | |
| | | | | | 1664 | 25149 | CGTGACGTCCCGCAGAGGCCTCGGAATTACTCAGCATTCACATGCCGTTT |
| | | | | | | 977 | |
| | | | | | 1665 | 33903 | CGCCCAGCCCCATGGTCCTGGAGAGTCCCCCGCTCCAGCCCTGGACCCTC |
| | | | | | | 32 | |
| | | | | | 1666 | 91580 | TCTGTCACCCAGGCATCAGGATTTTTCAAAATTACCCCTCTGTTGAGCCG |
| | | | | | | 280 | |
| | | | | | 1667 | 37608 | CGGCCATGACTAGATTTTCAAAGGCTATTGGTATCAGGAGTGTCTTATGA |
| | | | | | | 096 | |
| | | | | | 1668 | 29967 | CGCTGCGTGGGAGTTCCCATCCCGCGGTCCCAGGACCCCCGGCAGCAGCT |
| | | | | | | 531 | |
| | | | | | 1669 | 11104 | GGAGCTCTGACCTGCCCTGGCTTGGCTGTGCCTGGGTTTGGGACAGTTCG |
| | | | | | | 0242 | |
| | | | | | 1670 | 11062 | AATTCACCTGAGGCACTTGTTAAACCCAACTTTCCCAGGCTCCTCGTTCG |
| | | | | | | 7980 | |
| | | | | | 1671 | 31214 | CTGGGCTGGCTCTCACTGGGTTTATTGGAGCACCTAGGCTTAGAACCTCG |
| | | | | | | 401 | |
| | | | | | 1672 | 50265 | CCATTTGCTTATTCTCACTATTGTTTTAATTTATTGTGCAGCCTGTTGCG |
| | | | | | | 600 | |
| | | | | | 1673 | 10220 | TTGCTGAACCCTGCGGGGGGCCCGGCTGAGTTTGTTCATTTATGGAAACG |
| | | | | | | 802 | |
| | | | | | 1674 | 11931 | TGTAACTGCGCATTTTTCTAGACCAGTCTTTATTATTCCAGCAATCAGCG |
| | | | | | | 2767 | |
| | | | | | 1675 | 14714 | GAAACTGCAGCGTTTCGGCCTCACAGATGGGGCTGGAGTTGGAGACGTCG |
| | | | | | | 0880 | |
| | | | | | 1676 | 11495 | GAAGGTCCTCTGCACCAGAAAGGCTGCCCATGGCACAAACCCTGATCTCG |
| | | | | | | 7471 | |
| | | | | | 1677 | 89520 | CGCAGACCACAGAATCCAAGAGTGCTAGGAATCAGAGGAGAGAGAGGAA |
| | | | | | | 29 | G |
| | | | | | 1678 | 14042 | CGCATTTTGTCGGAATAAACCAACACACGGAGGTTGGGTAATCTAGCCCC |
| | | | | | | 0518 | |
| | | | | | 1679 | 12987 | AGCAAAGTTCCTTTCTAAATACCCCATATTGAATAAACACTGTTTAAACG |
| | | | | | | 1059 | |
| | | | | | 1680 | 13494 | ATCTCCAATGCAAACCCAGTGTGTGGGTGGCTGGAAAACACACTACTACG |
| | | | | | | 4446 | |
| | | | | | 1681 | 32173 | GGTTTCACATTCCATTGAAGTCCCACTGGCAGATGCCTTTGGTAAGCTCG |
| | | | | | | 227 | |
| | | | | | 1682 | 12130 | AAAAAGGTTTACATGACCACTAACGCTAACGGAGCACTTAGTACTTGCCG |
| | | | | | | 0857 | |
| | | | | | 1683 | 27015 | AACAGAGCCAGCATCTTGCCAGGGCCCCGGAGGAGGGGTTCCCCGCTACG |
| | | | | | | 813 | |
| | | | | | 1684 | 11637 | TTTTCAGCAGAACAGACGCAGCCCCCGAGTCAGTGCCGTCAGAAGACACG |
| | | | | | | 1187 | |
| | | | | | 1685 | 11270 | TATGTTGCTGGTGCGGCGGCTCCGCGGCGTTGCTGGCGGCGGGGGGAGCG |
| | | | | | | 9411 | |
| | | | | | 1686 | 17363 | CGCCACCCAGAGACTGAAATTATAGATAATTCCTGGCTGTTTTCCACTCT |
| | | | | | | 9130 | |
| | | | | | 1687 | 16877 | TATTGATAAGGACATTTCATACCTAACAAGGGCCAGGTGACATTTCCTCG |
| | | | | | | 3765 | |
| | | | | | 1688 | 15583 | TGACCAGAACTGCTTTTATTGACACCCCCTTTCCTAAACATAGCTTTTCG |
| | | | | | | 1789 | |
| | | | | | 1689 | 20359 | CGCCCCGCCAGCCTCTCCGCCGCTGGGCTCCACCCTCCCGGGCTCTGCTG |
| | | | | | | 8761 | |
| | | | | | 1690 | 43865 | TATCGACGGGTAAAAAGTGTTGTCCTTTAAAAAGTGCCCCACTAACTTCG |
| | | | | | | 055 | |
| | | | | | 1691 | 87229 | CGCGCTGGAGGTGAGACTAACCTCTAGTCCCCCGTCGAAGCCAGAGAGCA |
| | | | | | | 552 | |
| | | | | | 1692 | 77734 | CCTGCCACATGCAGATCACTTGGGCCAGCGGAAGTGCCCCTGGCTGCCCG |
| | | | | | | 334 | |
| | | | | | 1693 | 48918 | CCCTTGCAGCAGCGCTTTTCCGGGCGGGGGGCATCCGGTGGAGGGCTGCG |
| | | | | | | 116 | |
| | | | | | 1694 | 37321 | GCTGGGTGGGAAGCAGACGCGGTACGGTGGGCAGAGGTCCCCAGCCTGCG |
| | | | | | | 490 | |
| | | | | | 1695 | 50694 | CGCCTTGTTTGGATTAGCCCTCCAGATCTGCCAACCTGATCAACTCCTGA |
| | | | | | | 985 | |
| | | | | | 1696 | 10352 | CGCGCCCCAGAAGCTCTCAGATACCCCCACGATTTCGGTGACCTTCCGCA |
| | | | | | | 4899 | |
| | | | | | 1697 | 26223 | GGGCGGGAAGCCGGGCGCCTCCTTATTTCTTTACGGGGGCGCTGGCTACG |
| | | | | | | 206 | |
| | | | | | 1698 | 72668 | CGCCCACTGGCTTGCTGGTTAGGCTGTGATCGCGATCGCTTTGACTTCTC |
| | | | | | | 487 | |
| | | | | | 1699 | 79919 | TGGCCGCGGTGAGAGCGGGGCCCCAGGGCTGCGCGAGGTGGGGCGATGCG |
| | | | | | | 578 | |
| | | | | | 1700 | 43741 | GGATCGAGGCCCAGAAAACGGAGCAGCGGGCACCAGGGAGGCCTGGAAC |
| | | | | | | 557 | G |
| | | | | | 1701 | 10456 | GGCTTCACCCTTGAGACTGTGTTGCCCATTGCTCGGCCATCGGTCTCTCG |
| | | | | | | 9653 | |
| | | | | | 1702 | 51522 | GGGAGATTCGGGCTGGAACAGCGGTAATGGGCACAATTACCCTAATGACG |
| | | | | | | 588 | |
| | | | | | 1703 | 63025 | TTTCTCAGCCTGGCTTTTATTTACACTCTTATTAAGGAGGCTTTCATTCG |
| | | | | | | 948 | |
| | | | | | 1704 | 61835 | GTGGCAGTGCCACCTGGGCCCGGACTCTGGGACTGCGGGAGGCGGGAGCG |
| | | | | | | 708 | |
| | | | | | 1705 | 21666 | AGTTCCCCCTTTTGTTCCAAGTCCCGGAGCAGACAAAGAGGCGGCGAGCG |
| | | | | | | 659 | |
| | | | | | 1706 | 12419 | CGAAGTCAGGGAATAAGAAATCTCCAGTTCAGATCTCACAGCTTTGAAAG |
| | | | | | | 0997 | |
| | | | | | 1707 | 51139 | CGCCAGGCTCCTGACCCCAGCTTGGACAGCAGGCCGCCGAGCGCCCCTGA |
| | | | | | | 025 | |
| | | | | | 1708 | 26506 | CGCTCCCGCCTACCCGCAGCCAGGAAACCCCGACTCCGCTGACTGGGATG |
| | | | | | | 713 | |
| | | | | | 1709 | 99594 | CTTGGTGGAGCGAAGTTGCTCTCCCGGGCTTACCTGAACTGCCTGCGCCG |
| | | | | | | 931 | |
| | | | | | 1710 | 31938 | CGGTGTCTCCACTTCACTCAGGTATAGTGTTCCCTGGAACCGGGAGGCTG |
| | | | | | | 777 | |
| | | | | | 1711 | 10256 | CGAGCCTTCCGCGGCTTGGTTGGGGCTTCTCGGTCTTAACGTGGTTACCG |
| | | | | | | 9894 | |
| | | | | | 1712 | 23309 | AAATCCTGACATAGATTTCAACACACCTTATGATTACCAGAGCACAGACG |
| | | | | | | 774 | |
| | | | | | 1713 | 50260 | ATGCCATAAAACAACAAGCCGGTCCCTGGGAGCAAGTAAAAATTTATTCG |
| | | | | | | 129 | |
| | | | | | 1714 | 46916 | ACACCACGTTTCCAGCTTGGAGTGGGCCTTGAGCCTTGGGACTGACCTCG |
| | | | | | | 520 | |
| | | | | | 1715 | 13922 | CGGGTGCCGCAGTAACGGCTGCTGCTCGGCCAGGCGGTAACTGATGGGCG |
| | | | | | | 8150 | |
| | | | | | 1716 | 15780 | ATCCTGAGATGAGGTGGGTTGGCGACTAAGGCGCACCGGTGGGCACTGCG |
| | | | | | | 306 | |
| | | | | | 1717 | 39377 | CCTGGGCAGTGAGCCTGTTTATAGACACCCTTCTCCCTTCCTTGGTGTCG |
| | | | | | | 790 | |
| | | | | | 1718 | 15385 | CGGGGGGTTTCCAGCCCGCCTCGGGCAGGAAACCAGGACCTCCCTGCACC |
| | | | | | | 9646 | |
| | | | | | 1719 | 59990 | CGCCACCTGACCGGGCCTGGCGCCCCCAATGTGGTGCAGTGGCCCAGCAA |
| | | | | | | 794 | |
| | | | | | 1720 | 78982 | TGCCCCCTAAACTGTTGCAAGTCATAGCCCAAACTCTGTAAGCTAAATCG |
| | | | | | | 418 | |
| | | | | | 1721 | 81106 | GTCCCCTTGCGAGGGCACTAGGACAACAGGAGGGGCGCACCTGCAATACG |
| | | | | | | 857 | |
| | | | | | 1722 | 13414 | CGCCCTTTCTGCCGACCTCACGGGCTATTTAAAGGTACGCGCCGCGGCCA |
| | | | | | | 3906 | |
| | | | | | 1723 | 38624 | CGCCCCGCCCTCGCCACCCAGCCTCCCTGGCGGCCTCAGGTCGCGCCTGC |
| | | | | | | 28 | |
| | | | | | 1724 | 41174 | ACATGATGCCCTAGTGCAGCTGCTATGTGCATTTGCCAGACATTTCCACG |
| | | | | | | 793 | |
| | | | | | 1725 | 13060 | TCTGTCCAGGATACAGCCTGCAGCCAGGATCGTCCCTCTCAGAGTCCTCG |
| | | | | | | 741 | |
| | | | | | 1726 | 76227 | CGAGGCCAGTGCGCGGGCAGGAGCGGGGACGTGCTCAGAAGAGCCGGGC |
| | | | | | | 996 | G |
| | | | | | 1727 | 28544 | TTTGACAGCGCTGCCAGATTCGGGAATGAGGATCACTGCGGCGAGGCCCG |
| | | | | | | 760 | |
| | | | | | 1728 | 79495 | GCCGGCTCACGCTCAGCAGGTGGGCCTGCGGGTGGATGGCCAGGTGCACG |
| | | | | | | 953 | |
| | | | | | 1729 | 24803 | CGGCCCGGCGGGCCCCACCTGAGCTTTTCTCACCCGCTCAAAGCCGCTAC |
| | | | | | | 917 | |
| | | | | | 1730 | 12409 | CGGAGTGACGTGAGGTGAGATTTACAGGCTGTTCAGTACATATCACAAGT |
| | | | | | | 2424 | |
| | | | | | 1731 | 41881 | CGTCTTCACTCAGGGCTTTGGGGATCCCCTCGTCGCCCCAGGTCAGTGGG |
| | | | | | | 829 | |
| | | | | | 1732 | 14042 | CGGCCAATACCTCGGCTTGCACAAAGCTGCCTGTCAGCTTTCCTAAGACA |
| | | | | | | 0475 | |
| | | | | | 1733 | 14572 | CGGGAGTTTGTAGTATTCATAACCGTTGAGGACTCTCTGACCCCAGCTCG |
| | | | | | | 0805 | |
| | | | | | 1734 | 79448 | CGGGCCTATCATTGGCTTAGGTTTTAGGAAGGTCCCAAGCTCATTTGCCA |
| | | | | | | 940 | |
| | | | | | 1735 | 28501 | GCGAGCGCAGATTTGCCCCACCCCAGAGCCCAGGCGGGTGTGGAGAACCG |
| | | | | | | 366 | |
| | | | | | 1736 | 21984 | CAATCGTCTGGCAGAGTTGGCCAGCAGAGATCAGCCGGCGTGAGTCAGCG |
| | | | | | | 8528 | |
| | | | | | 1737 | 12589 | CGGGTCTGCACCAACCTCAGTTTCGGGGCTGCGGCTCACAGAAGTCTAAC |
| | | | | | | 9401 | |
| | | | | | 1738 | 44422 | CGGGGAACATCAGCACCCACGGAGACACCTGCGCCAGGGAGACACCTCTG |
| | | | | | | 8 | |
| | | | | | 1739 | 53359 | CAAGCCGAAAACCAGGAGACTCTTGCAGTCAGGGGGCAACTGGGGAAGCG |
| | | | | | | 506 | |
| | | | | | 1740 | 49576 | TGAGGCCTGACGTCCAGCAACAGGTGGAATGCCCCAGAATCCAGCAGTCG |
| | | | | | | 406 | |
| | | | | | 1741 | 15267 | GACAGGTCCTGGTTGCAAGTTTCTCCATAAGGGTTGCTCTGTGATGTTCG |
| | | | | | | 1707 | |
| | | | | | 1742 | 56562 | GACCTAGATGTGAGCCACACCTTCACAAGCCATGCCATGTTCAGAGAGCG |
| | | | | | | 731 | |
| | | | | | 1743 | 75315 | CGGGACAGGGAAAATGAAAGACTTTGGAAGTCGTCAGGAATTTGACTCTG |
| | | | | | | 837 | |
| | | | | | 1744 | 10862 | CGCTCCAATTTGAGATCCCAAAATGAGATGCTGCCCGGGACCAGAAGTCA |
| | | | | | | 20 | |
| | | | | | 1745 | 29847 | CGCCACCCTCCAAAGGCGCTGTTCCTGAGAGCCGGTGCCTGCCTGCCCAC |
| | | | | | | 171 | |
| | | | | | 1746 | 77935 | TGTCAGCTTTTGCAATTATTGGAATGATGTTCACCTGTAAAACAGACACG |
| | | | | | | 993 | |
| | | | | | 1747 | 90689 | CGGGAAGTTACTTAGCACGGTTCCGGGTTTCTCGCGCCCCGCCTGTCCCC |
| | | | | | | 603 | |
| | | | | | 1748 | 60386 | CGAGACCTGCGGTTGCTTTGGTTTGCCTTTTACTAAGTTCATTTCTCAGC |
| | | | | | | 050 | |
| | | | | | 1749 | 85672 | CGTCCTGACACAGCCTTTGTCAACACTGGCACTCACAGCGGCCGTCGCGT |
| | | | | | | 623 | |
| | | | | | 1750 | 17629 | ACAGGGTGGCGCAGGCTGTTTTCTGGACATTCTGCCCCTCACAGAAAGCG |
| | | | | | | 6727 | |
| | | | | | 1751 | 25033 | CGGCGATCCGCTCTCTGCGCCCTGGGAAAAAGCCCCAGCCATGAGCAATC |
| | | | | | | 893 | |
| | | | | | 1752 | 10288 | CGTTTCATCGGTAGTACCTTGCCCCAGTCGCTGGCTGCCCTTTGCCCTGT |
| | | | | | | 0841 | |
| | | | | | 1753 | 45025 | CCGTTTCAAAACTGTTCTAGCAAGCACTGAAGGAATGCATAGGGTCACCG |
| | | | | | | 080 | |
| | | | | | 1754 | 95155 | CCCCTGCTTAATTTCCTGGGTGTTCGTATTTTAGGCTCTTTGCTTCCTCG |
| | | | | | | 784 | |
| | | | | | 1755 | 19018 | TTTTCCTAGTAATTTATTCTTCAAGGCCCATCAATGTAGTTATAAAAACG |
| | | | | | | 055 | |
| | | | | | 1756 | 24641 | AGCTCTGCATCTCCAACCTGGAACCCAACCCAGAAGTCTCAAGTTTGACG |
| | | | | | | 501 | |
| | | | | | 1757 | 18329 | CGTCAGAGCCAGGGCCCCCAGTTGTGGAAGTTTCTATATCCGGTAAATTG |
| | | | | | | 562 | |
| | | | | | 1758 | 14286 | TTCCCGGGCTTCAGCACAGGTGACTTGGCTGAGGTAGGTGGTGGCTGACG |
| | | | | | | 39 | |
| | | | | | 1759 | 25034 | TGGAGTCTCTCTCCTCCACGTGCTGAGAGGCGCCCTCTGATTGGCTCTCG |
| | | | | | | 151 | |
| | | | | | 1760 | 17443 | CGCTGCGGCCCCCGCTACCGACACCGTCTCGACCCCGCGCATTCTATCCG |
| | | | | | | 0614 | |
| | | | | | 1761 | 11944 | CGCACGTCGCTCCCGCTGCAGCCAGGCCGCTCCCCTGGGACTCTGGTCCA |
| | | | | | | 4909 | |
| | | | | | 1762 | 11707 | CGGCGGCAGCCCTTTAAACCCCTCACCCAGCCAGCGCCCCATCCTGTCTG |
| | | | | | | 0046 | |
| | | | | | 1763 | 37123 | CTCCTTCCCGCGGGGGCTCTTGGGAAGATTCTACAATATTCTAGAGGCCG |
| | | | | | | 671 | |
| | | | | | 1764 | 38066 | CTTCTTCAAAGCCCAAGACCGGTGATGCAGAATGGACTCTGCCAGCTTCG |
| | | | | | | 873 | |
| | | | | | 1765 | 62321 | CCAGGACTACGGAGCTGTCTTCCTCTGTGACCACAGGTGCGTGCAGTCCG |
| | | | | | | 231 | |
| | | | | | 1766 | 10089 | CGGGCGTGGTCAGCACTGCTTTGGGGCTAAGCAGAGTTTCAACATGTTTC |
| | | | | | | 6674 | |
| | | | | | 1767 | 82978 | CGGCACTGCCCAGGGTACACACACCTCACCGACCACGCACGCCGGGCAGG |
| | | | | | | 3 | |
| | | | | | 1768 | 44063 | CCCGAGGTGGCAGTGGGCTCCTGGTACAGCGGATGCGAGTCCGCACTCCG |
| | | | | | | 404 | |
| | | | | | 1769 | 11810 | GGCAGCGCTCGCTGGACAGTGAGGAACTCGCGGAGAAGATGCGCTTGCCG |
| | | | | | | 7876 | |
| | | | | | 1770 | 41260 | CGCACCCGGCCAGAACCCTGGTTCTTTAGGACAGTGACAGCCCAGAGTAG |
| | | | | | | 294 | |
| | | | | | 1771 | 15112 | CGGGATGACTGCAAAGCATTTGTATGGATGGCCATCTCCAATCATCCCTA |
| | | | | | | 8222 | |
| | | | | | 1772 | 43211 | CGCGCCGCCAGCATCAGCACCAGCCCTGACATCACCGCCCGCCGCCCGCT |
| | | | | | | 213 | |
| | | | | | 1773 | 18254 | CGCGGCGCTAGACAACCTGCGCAAGGTGGTGCCTTGCTATTCTAAGACGC |
| | | | | | | 3233 | |
| | | | | | 1774 | 39188 | CGCCCTACAGCCCGCACAGCCCGCACGGCCCTGGTATGGTGGCTCTGCTT |
| | | | | | | 743 | |
| | | | | | 1775 | 16228 | CGCACTGACAGTGTCATTTCACCAGACACCCAAGACTTCTTGGAAGCGAG |
| | | | | | | 4135 | |
| | | | | | 1776 | 11121 | GCATCGGGTCTGTCCGCAGCCGGCAGCGGTGCAGAGCGGTGGGGAGAGCG |
| | | | | | | 6962 | |
| | | | | | 1777 | 26757 | CAGTTACAAGAAGCCGAGAGGTCTTCTTGTCATAAAATTAACTTCCCTCG |
| | | | | | | 706 | |
| | | | | | 1778 | 21000 | CAGAGGGAAGCCGGGGGGTCTGTGCTGGGATCTGAAGGTTTGCAGCCTCG |
| | | | | | | 992 | |
| | | | | | 1779 | 11944 | CGCCCCCGGCTCTGGGCGCCCCGCAGAGCATCCTACTCCGCGGTTGCCTC |
| | | | | | | 5209 | |
| | | | | | 1780 | 52536 | CGGCCGCTAGGGAAGACTTGGCCACCCTCTGGAAGTACCTTCCCCCGGGA |
| | | | | | | 436 | |
| | | | | | 1781 | 13473 | TTTCCGCCCGTGTTGCAAATGCCCGTGGTTGGGTTGGGGGCGGGACAGCG |
| | | | | | | 1585 | |
| | | | | | 1782 | 70235 | TACCGTCTATCATTTCGAGGTCTGCGACTACTAAAACGGTCAGAGTATCG |
| | | | | | | 567 | |
| | | | | | 1783 | 21251 | CGCCCGGCACCTGATCCCTGCAGGGCCCCCAGGGTGGAGCAGCAGCCGTC |
| | | | | | | 86 | |
| | | | | | 1784 | 77089 | CGCGCTGGCAAGTTTGTGGAACACTTTCTAGGAATTAGGTCTTTTCCTCC |
| | | | | | | 518 | |
| | | | | | 1785 | 50886 | CGCCGCCAACGGCATCATCCCCCCGAGGCCCGCCTACGAGGTCTTCGGTT |
| | | | | | | 782 | |
| | | | | | 1786 | 85667 | CGGTGTCCCCGATCGCGCTGAACCACTTCCCAAAGCCTGCGCCCTCGCCC |
| | | | | | | 616 | |
| | | | | | 1787 | 21926 | CGGGATGATACTCTTTCTGGCCAACATCGTCCTGTCACCATAGCCAGCTT |
| | | | | | | 524 | |
| | | | | | 1788 | 73822 | TGGTGCATGAAAGCAAAACCAGAGAGGCGCCTTTTCCAAATGCCAATGCG |
| | | | | | | 8 | |
| | | | | | 1789 | 11154 | GCTGGGCAGGAGCTGAATTCCCGGGAGCTTGCCTGTGTAGACCCAGTGCG |
| | | | | | | 4463 | |
| | | | | | 1790 | 17357 | AAAGGGCTTGGCCTGGCGGCCGGCTGGACTGCAGATGTCTCTTCCAGGCG |
| | | | | | | 619 | |
| | | | | | 1791 | 54852 | GCTGTGCGTTTGACAGGCAGTTGGGAGACCCTGGTGGCCGGTTTTGGTCG |
| | | | | | | 84 | |
| | | | | | 1792 | 14710 | GGGCGACACGAGGGCAGACGGTGTAGCCGAATCGTAGCCAGAGCTGGGCG |
| | | | | | | 8843 | |
| | | | | | 1793 | 62795 | CATTAGCAGCGGTGGCACGGGCTGGCGGAGGAGGCTCCTTTGTAAATACG |
| | | | | | | 807 | |
| | | | | | 1794 | 80992 | CGGCACTCAACTGAAGGCTGATATCAGGCGATTAGACAGCCATGCATTCT |
| | | | | | | 750 | |
| | | | | | 1795 | 83680 | TCTCTTTAGTCACCACTCTCGCCCTCTCCAAGAATTTGTTTAACAAAGCG |
| | | | | | | 326 | |
| | | | | | 1796 | 85996 | ACTAACTTAACCCTGTGTGCGCCTGACACTCCCTAGAGAAGCTGGGATCG |
| | | | | | | 364 | |
| | | | | | 1797 | 11561 | AGGCTCTGGGGTCTCCGCGGAAAATGCCCAAGTGCTACCGCCAGGGAGCG |
| | | | | | | 498 | |
| | | | | | 1798 | 29237 | CGGCCTACCCCAGCCACCCCATGCCCTACAGCTCCGTGTTGACTCAGAAC |
| | | | | | | 480 | |
| | | | | | 1799 | 20125 | CGTGGTGTTTTACCGGGGTATACTCATGAACAACAAATAACCACCGGTTT |
| | | | | | | 1982 | |
| | | | | | 1800 | 67506 | TCTGGTTCAGGAACTTGGGACCCTTCATTATCCCAAACGTTTGAGCTTCG |
| | | | | | | 17 | |
| | | | | | 1801 | 69420 | CGGCACTGTGGAGAGAGGACTTGGAAAATAACTAAGTTGGTTTAAATTGC |
| | | | | | | 45 | |
| | | | | | 1802 | 30833 | CGGGGCGCCTCCTCCACGCAGGCCTCCGGCTTCCTCCAGGCGACCAGGCC |
| | | | | | | 33 | |
| | | | | | 1803 | 12991 | CGCCTCAACCCAGCAAGGCTCAGGACTCTTCCATGCGTGGGCACACGAAG |
| | | | | | | 665 | |
| | | | | | 1804 | 10184 | CGCCCGGCCCAGAGCATTCTTTATAAGGCCGAATAGTTTGCATTTGAAGG |
| | | | | | | 319 | |
| | | | | | 1805 | 62626 | GCTCAGTTCCCCCTCTCTGCTCATGGTTACCTAGTGTGGTCCTCAGCTCG |
| | | | | | | 20 | |
| | | | | | 1806 | 66664 | CGTCTGGGATGAATGATCTTGCCCTAACTTGTGTCCCTATCAACTAGTTT |
| | | | | | | 667 | |
| | | | | | 1807 | 15137 | CGGGACTATGCAAGCAAGGAGGGAGAGGAGCCATTTGCAGGTTTCACACA |
| | | | | | | 4946 | |
| | | | | | 1808 | 88271 | CCCTGAAAATCAAATGGCAGAACCTAAACGGAAGACTCACTGCCAAAACG |
| | | | | | | 572 | |
| | | | | | 1809 | 30734 | CGGCCCCGACGCCCTCCAGGGCTGAGTACCTACACTCACACACACGGCGG |
| | | | | | | 41 | |
| | | | | | 1810 | 11069 | CTTAGCTTCTTGAGGCCCCTGGTCAGGGGATGCAGACCCTGGAGCCCTCG |
| | | | | | | 2918 | |
| | | | | | 1811 | 30095 | CGGTTGCTTTTCAAATACAGCTTTTCTAAAACGCCGCCAGGCACCCGGGC |
| | | | | | | 326 | |
| | | | | | 1812 | 29186 | CGCTCCTCTAGGCTCACATGACCCAGACCCACAGGGCAAAAAGTGCGTCG |
| | | | | | | 183 | |
| | | | | | 1813 | 85402 | CTTCATACTCCCAGACCCAGACAGAGAAGTTTCCCGAGTCATCCACATCG |
| | | | | | | 497 | |
| | | | | | 1814 | 92191 | TTGCTCATCCCAGTGCAAGATCTGTTTATGTTCCTCTCCTTGGCTGTACG |
| | | | | | | 625 | |
| | | | | | 1815 | 69535 | CCTCTGAGCAAGTTGATGGCTATCTCAAAACCTCGAAACCTGTCTCTACG |
| | | | | | | 58 | |
| | | | | | 1816 | 49815 | CGTCCACGCAGCCCTTGCCACGGTAGTCCGGGTACACGTACTCCTCGGGT |
| | | | | | | 502 | |
| | | | | | 1817 | 15975 | TGCTCTCACCAGGCTGCGCGCTGGCGGGTCGGTGCACAGGGACGGGGTCG |
| | | | | | | 6929 | |
| | | | | | 1818 | 34492 | CGGGGCCCTGCACCTGTGCAGCGAAGCTTAGCGTTCATCCGTGTAACCCG |
| | | | | | | 984 | |
| | | | | | 1819 | 65284 | CATTTAGTTCCATCGAAAGGGAATGTAACAATATAACTCACCACTTCCCG |
| | | | | | | 438 | |
| | | | | | 1820 | 10031 | CGGGAGCCCAGCCTTTCCCAGATAGCACGCTCCGCCAGTCCCAAGGGTGC |
| | | | | | | 8080 | |
| | | | | | 1821 | 11513 | CGGACCATTGTGGCTTGCACAAAACCCGCGCCCCAAGAGCCTCTGCCAGG |
| | | | | | | 6226 | |
| | | | | | 1822 | 17713 | CGTCCACCACGACCATTTCTCCCGGGAAGTCGTTGCGCACCGAGAAGAGG |
| | | | | | | 3566 | |
| | | | | | 1823 | 18707 | GGCTCTATCTCCTTGTGGTCATCATAGCCCATTTGGCCCTGGTCAAGTCG |
| | | | | | | 479 | |
| | | | | | 1824 | 70528 | GTGTGCGTGGGGGGTATGCAGCATCCAGTTACATTCCTGACCCACTTTCG |
| | | | | | | 60 | |
| | | | | | 1825 | 12175 | CGACAAGAAAGATACTGAATATCGCCCTCCGAAAATCGGGCGCATTTGAA |
| | | | | | | 8980 | |
| | | | | | 1826 | 49518 | CGGGTGGAGGATTATCAGGTAACACAAGAAGTGTTGCAGACCTCCTGGGC |
| | | | | | | 986 | |
| | | | | | 1827 | 29186 | ACCCTGGGGCTGCTTGCTTCTTCCAGCATGGGAAGCGCTGACTTGATGCG |
| | | | | | | 267 | |
| | | | | | 1828 | 17696 | CGGTGATTTTGAGTCACCCAAGATAAGACACTAACTTGACCTTAACTTTG |
| | | | | | | 9218 | |
| | | | | | 1829 | 20656 | CGGCTTCAGGGATGTCGTAGAGTCTTAGCTACGAATCATCCAATACCCGC |
| | | | | | | 2 | |
| | | | | | 1830 | 18144 | CGCGGATTGCCGAGGGCTTGCAGGCACTCAGGCAGACGCAGACAGAAACC |
| | | | | | | 4989 | |
| | | | | | 1831 | 18891 | CGGGGCCCCCTGACCCCCGCGTTCACCCCTCAGCCCGGCCGCCGGGCACT |
| | | | | | | 6668 | |
| | | | | | 1832 | 28862 | TGGAAGAAACGTCTTCTTCCTGCCAAAACAGACACGCACTGGCTGTCTCG |
| | | | | | | 95 | |
| | | | | | 1833 | 54956 | TCCTTCAGTTAAATAAGAAATCAGGTCCAGAGCCGAGTTCAAGGCATTCG |
| | | | | | | 419 | |
| | | | | | 1834 | 29323 | CATCCAGTTCCCTGCCTCGGAGATAAAGATTCCAGCTACATGGGCAAACG |
| | | | | | | 182 | |
| | | | | | 1835 | 51417 | CGGGGGAAGGGCAGCAACTCGGCGCCCCCCTCAGCGCCTCGCACTACCTC |
| | | | | | | 469 | |
| | | | | | 1836 | 35086 | GGGTCCCGAGTGGGACGGGGGGCTCGGCGGGAAGTTTACCTGGTGCCGCG |
| | | | | | | 890 | |
| | | | | | 1837 | 74799 | AATCATCTGGCTTCTGCAAAGCAGTCGCGCAGCGCTGTAGGGTGAGGCCG |
| | | | | | | 572 | |
| | | | | | 1838 | 11930 | TTACAGACTGTCCTGGCGGTGTCTGCAAGTCGGTAACCCCCCGGGTTTCG |
| | | | | | | 4487 | |
| | | | | | 1839 | 14712 | CTGCCCTTCCCGAAACCAACTTTTCGCTGAGCGATTAGAGACTTCAGACG |
| | | | | | | 6703 | |
| | | | | | 1840 | 67218 | GTCCGGCCGGGGAGGGCCGAGGGCTCTGTTTGAGGTCACCCTGGAGACCG |
| | | | | | | 135 | |
| | | | | | 1841 | 63743 | CGGCCCATCCTGGTCATGTTACTGCATTTGTGGCCGGCCTCCCCTGGATC |
| | | | | | | 837 | |
| | | | | | 1842 | 10006 | CGCGCCTTCCCAGGCAGGAACTTTGCGACCCCACCCCCTGGGCCCGCCAC |
| | | | | | | 1111 | |
| | | | | | 1843 | 67090 | CAGGCGCTTCGCAGGTGAGCCGGGTGCTGCCGCCTCTCTGCAGAGAGACG |
| | | | | | | 581 | |
| | | | | | 1844 | 10335 | CGCAGCCAGCCCCACCGCCTCCGGACTCGACCCCACCATAGCCCCGCCGT |
| | | | | | | 9572 | |
| | | | | | 1845 | 26250 | CTATTTATAAAGGCAAAGTCCTTCTGATTGGTCCGAATCTTCCAGTTTCG |
| | | | | | | 934 | |
| | | | | | 1846 | 14646 | CGGGTTATCTGATGGAAGTAGTATAGAGTCATCATTAAAAGTAGAGACTT |
| | | | | | | 1855 | |
| | | | | | 1847 | 15402 | CGTGGCTGATCACCCCAAGAGAAGTCAACCCCAGGCGGGCGGTTACGACG |
| | | | | | | 6930 | |
| | | | | | 1848 | 10100 | GAGGTCTGGCTTTCGGGGTGTTAGGCTCCAGGGACCTCCTGCGGAGCTCG |
| | | | | | | 6930 | |
| | | | | | 1849 | 13561 | AACATACAGGCACAAAAGGGCACATACACACTCTCAAAACCAGAATGCCG |
| | | | | | | 5376 | |
| | | | | | 1850 | 10091 | CGGGGCCGACTCGTGTCACTCAGCCCTATTGGCCAATGAGCGCCTCGCCC |
| | | | | | | 2946 | |
| | | | | | 1851 | 23350 | GCTTTTCCCTCCGCGATCATGGTGAATCAGCAAAGCGCTGCAGAGCAGCG |
| | | | | | | 552 | |
| | | | | | 1852 | 27259 | CGCCACAAAGACGTCAGGGATTAGGGTCGCAGGCGCCTTGTTTAATGGCC |
| | | | | | | 010 | |
| | | | | | 1853 | 29760 | TGGGGAGTAGCTAGGGGCCTGCCCGGCGGGGGCGCAGGAACCCGGTTGCG |
| | | | | | | 164 | |
| | | | | | 1854 | 38080 | CCCCAGCAGAGCGAAATATCCTGAAGTAGCTTTCTTTGCCCCAATTTGCG |
| | | | | | | 364 | |
| | | | | | 1855 | 85996 | CGTTCGAAACCTGTCGCCGTCACTTGCGCGTTTGGCATTATCCATTGTCA |
| | | | | | | 495 | |
| | | | | | 1856 | 33169 | AGCCCACCGGAAGCAAAACTGAGCAAGATCTGAAGTAGAGAGCGATGCCG |
| | | | | | | 600 | |
| | | | | | 1857 | 12405 | CGGCTCAGCCCCATAAGGTAGAGCAGCATTTTGGGACAAGTCACTGAGGA |
| | | | | | | 6244 | |
| | | | | | 1858 | 14714 | CGGCGCATGCTTGGGACTGAGTCTACAGAACTACTTCTGGAAAAAATCCT |
| | | | | | | 1588 | |
| | | | | | 1859 | 87975 | CGGCACTTCTGTGTTCATGTGTGTACAAGGTGGTATGTAGCCCACGCATG |
| | | | | | | 812 | |
| | | | | | 1860 | 11381 | GAGAGAAAGTTTGCTAGCCACGCGGCTTGCTGAAGCACGTCTGGGCTTCG |
| | | | | | | 6304 | |
| | | | | | 1861 | 15725 | GGACAAGTGGCTGTGAAATCGATTGTGAAGCGTGACCTTCCATCCCAGCG |
| | | | | | | 196 | |
| | | | | | 1862 | 14712 | CCCTCGATTAACCTGTCTTTGCCTAGCGCAAAGTCTATTCAACAACTGCG |
| | | | | | | 5714 | |
| | | | | | 1863 | 76924 | CGAGAGTTCCTCAGATGAACGGTGTCCCTGGGGCAGTGGCGCGGAGGGCG |
| | | | | | | 190 | |
| | | | | | 1864 | 57269 | CGCTTTACTCAATGCTCAGTGAGTCATGAGGTGGCTGCCCCCAGGCCTCC |
| | | | | | | 634 | |
| | | | | | 1865 | 68449 | CGACGAGGAAATACTAACAAGGGACAGCTCTGTGGTTTCTGCTATCGAAA |
| | | | | | | 0 | |
| | | | | | 1866 | 11944 | CGCCAAGTAGCGACTCTTTAAGCCCTGGGCCACCAACCAGCCCACTCCCC |
| | | | | | | 3277 | |
| | | | | | 1867 | 33715 | CGGGATCTGGCACACGCCTAAAGCCCTCTCAGTGCTATCTGTATTCCGTG |
| | | | | | | 66 | |
| | | | | | 1868 | 60992 | CGCCCGGCGGCATCTCCCGTTTTTTTGTGGCATAACAGTTGACCAAAATA |
| | | | | | | 50 | |
| | | | | | 1869 | 19615 | GCAAGTGCAGAAGGTCCTAGTAGCCGAGCGTGGAGTCGAGCTCGTGGGCG |
| | | | | | | 358 | |
| | | | | | 1870 | 45008 | CGGGACCTCATGCAGCTTGGCAGATATCTGAGAAATGGTTTAATTCATGC |
| | | | | | | 526 | |
| | | | | | 1871 | 60985 | GCCGGCCTGGCGGAGGGTCAGGTGGACCACAGGTGGCACCGGGCTGAGCG |
| | | | | | | 645 | |
| | | | | | 1872 | 21984 | CGGGCTTCCCAATCACTAGACCCTGGCTACTGCCACCTGCTTCACCCTCC |
| | | | | | | 8580 | |
| | | | | | 1873 | 45148 | AGTGAGAGAGCCGGATAAGTACCATTGCTGCTTGATTGCTGTCCAAGTCG |
| | | | | | | 557 | |
| | | | | | 1874 | 21439 | TTCGCGGAGGGGCGGGGCTGTGGGGGCTGTCTTCCTGTGTTTTCTCTCCG |
| | | | | | | 42 | |
| | | | | | 1875 | 32712 | GTCCATCTACAGTTTCGACGAGACCAAAGACTTGGATCTGGAAGACATCG |
| | | | | | | 623 | |
| | | | | | 1876 | 53312 | GGCTTTCCGCCTTAGGTCAACGTTGTAGAAAAGCAAGTGAAGGTTTCACG |
| | | | | | | 966 | |
| | | | | | 1877 | 11192 | GGGAGGCTGCGGTGCTGGAGCGAGGATGCGCAGACCGAGGCAGGGAGGC |
| | | | | | | 9547 | G |
| | | | | | 1878 | 11377 | CGGCAAGTGACATGACATCATTCATAAAGAAATGGGTATAAACAGGATTG |
| | | | | | | 8957 | |
| | | | | | 1879 | 25282 | CGGTTGCACCTCTGGGGTGGAACATCTATAAAATTTAGATAATGATACCC |
| | | | | | | 664 | |
| | | | | | 1880 | 11421 | CGGGATCCCAGTTCGTTTAATTGCACCGCTTAGTTTACATTTCAATGGCT |
| | | | | | | 4093 | |
| | | | | | 1881 | 16626 | CTCTGAAGTTTGGGCTCATTGCCAGTTTAAGTATATGCTGCCACCTTTCG |
| | | | | | | 2886 | |
| | | | | | 1882 | 49311 | CGCTCATTCACAGGACTTTATTCAAGAGGTTCGTCCAGGAGTTTTGATTG |
| | | | | | | 934 | |
| | | | | | 1883 | 16374 | CGTACAGATCTTTTCCAGAATAAAGCATTATTCGGCCAGGACGCCTCAGC |
| | | | | | | 6145 | |
| | | | | | 1884 | 10886 | CGGGGCAAAAACTTGGAACAGCTACCACAAAGCGAACGCCTATCTGTTTG |
| | | | | | | 967 | |
| | | | | | 1885 | 37136 | CGGCCCTTCAGAAGCGCCTTCTTTGGTTAACTCTACCCCCAACGAGAGCC |
| | | | | | | 192 | |
| | | | | | 1886 | 35870 | TCGGTGAGGCGGGTGGGCTGGCGGGAGCTCTTCCCGAAGTGCGTTCTACG |
| | | | | | | 38 | |
| | | | | | 1887 | 44325 | AACGAAGTATGCCATCTGTTTCCGTTATCAGTTTATTGCTCTCACCTCCG |
| | | | | | | 008 | |
| | | | | | 1888 | 96180 | CGGGACTCTGGGCCTCTGCTGGGAACTCACAGTCGACACCAGGATGCCCA |
| | | | | | | 847 | |
| | | | | | 1889 | 27858 | CGATGATCTGATCCTGATTGGTCAGAACTGTTGTAGGAAAAACCTGGTTG |
| | | | | | | 637 | |
| | | | | | 1890 | 46544 | CGTGACTCTGAAAAATTATAGACAGTGCAGGTCTGCAGGTGAACTGAGAC |
| | | | | | | 711 | |
| | | | | | 1891 | 20359 | CCTATTTTCAGTAATCTGATTAGGGGTCGATGTTGGTGGGCTCGGGGACG |
| | | | | | | 8594 | |
| | | | | | 1892 | 12194 | GCCCAGGTTCTACAGAGTCCAGGGATCTCCTTTCTGCACCCACCGTAGCG |
| | | | | | | 0381 | |
| | | | | | 1893 | 19574 | CGTCACCCACACAAACCTGACCTTCACGTGGGGCGCGGGACCTTGCGGGG |
| | | | | | | 96 | |
| | | | | | 1894 | 39659 | CGAACAACCCTGTTGAAGGAGAAGTTACCTTCTGTAGTGTGCTGGGAATT |
| | | | | | | 249 | |
| | | | | | 1895 | 20001 | CGGGTTCCCATAGCAGCCAGACCCACAGGTCAGCCTCCGGGGCTTCAGCT |
| | | | | | | 0137 | |
| | | | | | 1896 | 11568 | AACTTCTCTCTTTTCCTTAACCAAACACCAAATTCCCTCCAGGATTGTCG |
| | | | | | | 257 | |
| | | | | | 1897 | 11052 | CGGGTGAGGCATCGCTCCTGAAGAATTAATCAGAAACTCAGAAAAATTCC |
| | | | | | | 2297 | |
| | | | | | 1898 | 30908 | CGGTGATCTAAGCCCTGAGCCCAGGTCCCTGAAACCTCAGCCCCAGGCCT |
| | | | | | | 233 | |
| | | | | | 1899 | 20000 | TGTTGAGAGCAGAGTTCAGGGCTGGTGCACTCTGCGGTGCTGAGTGGGCG |
| | | | | | | 9927 | |
| | | | | | 1900 | 23581 | CCTGCGGTGCCACAGGGACGAGGCCTGGAGAGCAGTCGCTCCTAGAACCG |
| | | | | | | 4009 | |
| | | | | | 1901 | 48537 | CGGCCCCAGCCCATCAATGAATGGGGCTGAATCAACAGTGAACAAAAATG |
| | | | | | | 06 | |
| | | | | | 1902 | 15847 | CGGGGTGTTCCTGCTCCGTGGCCTCCATCCCAGCCGATCCCGCTTTGCAG |
| | | | | | | 8734 | |
| | | | | | 1903 | 23633 | CGGGAAACCACACGTCTAAAGCGTCCCATCTGTAAACTCTTGCTTACTCT |
| | | | | | | 680 | |
| | | | | | 1904 | 29912 | CGGAGTCTCAGCTACGAATCATCCAATACCCGCAGGTCACAGAGCGACGG |
| | | | | | | 1 | |
| | | | | | 1905 | 13780 | ACAAGATTGGATTCGCCCCTGCCACTTTAAGAACTCGTGAGTCTACCACG |
| | | | | | | 6993 | |
| | | | | | 1906 | 13530 | CTTTTGGGCGCGGCTCGGGGGTGGAAGCCCTGGGCGACAGCACAGCTTCG |
| | | | | | | 923 | |
| | | | | | 1907 | 10540 | GAGGCGGGTTGCGATGGCAGCGCTGGGCTACAGACCCCGCCTTATTTGCG |
| | | | | | | 1673 | |
| | | | | | 1908 | 11671 | AAGTGGGAGCCCTTCCCCCATCCCGGGCGTTGTTAAAACCCAGTGGGGCG |
| | | | | | | 1077 | |
| | | | | | 1909 | 16029 | AGCTCACCTGAGAATCTGCACACCCAGATGCAGGTGAGCACCGGGTCTCG |
| | | | | | | 14 | |
| | | | | | 1910 | 63784 | GTGGCTTGAATAAGTCGTTGGCGCGATGTGAGATAGAATCATCATGCCCG |
| | | | | | | 454 | |
| | | | | | 1911 | 79085 | CGCAGTTGTTCTGAGGAGAAATTAGGAAAAGGTATGCAGTTACACAATGC |
| | | | | | | 713 | |
| | | | | | 1912 | 32223 | CGTGGTATCTCGCAGTTTGGCTGAAACCTGAATTAAATGCAATGCTTTTT |
| | | | | | | 226 | |
| | | | | | 1913 | 25034 | GCCCGGACTGGATTCCAGAGGGGCAGGCGGGGGTGGGGGCAGCGGGCAC |
| | | | | | | 241 | G |
| | | | | | 1914 | 99275 | CGGGTCCCTCTGCTAGGACCGCGCCTTCAACGCGGAATCGCTGACATGAG |
| | | | | | | 335 | |
| | | | | | 1915 | 11913 | CGTGCTGTGAAACTGGACTTTGATTTAACCCCTGCGAGAGGAAATTCTGA |
| | | | | | | 0048 | |
| | | | | | 1916 | 56951 | CGTCCTGCCCAACTATGTAGCGTGCAGGCCTCCATCTTCCACATGACTGC |
| | | | | | | 337 | |
| | | | | | 1917 | 18891 | CCTCCCTAACCCTGGCGGAGCTGATGGGTGGCTGTAGCCTGATTAGACCG |
| | | | | | | 6865 | |
| | | | | | 1918 | 59001 | CGTCGAGGTGGCAGGGAGTTGCTGGGAGAGCACAGCGCGGAGAGGGACC |
| | | | | | | 158 | G |
| | | | | | 1919 | 11643 | CGCACCGGCCCTCCCCCAATCAGCCCCAGCAAGAGGAAGTTCAGCATGGA |
| | | | | | | 427 | |
| | | | | | 1920 | 50265 | TGACCCGGAGGACGCGCCTACGGAAGAGACGTCTGGTCGAGGGGGTGCCG |
| | | | | | | 443 | |
| | | | | | 1921 | 14020 | GATGGTGAGGACTACAAAGGTCTCCCCACCTCCCGGGGGCCGCGGAGGCG |
| | | | | | | 1433 | |
| | | | | | 1922 | 85997 | CGCAGGCGCTCTAGAAAGGCGTGATGGCCCCAGCTCAGTGCCCGCCAGCC |
| | | | | | | 519 | |
| | | | | | 1923 | 87240 | CTTGAACATGACCCTGCGAGGCGGGCCGGGAGGGGGCGTGGCATGCTGCG |
| | | | | | | 60 | |
| | | | | | 1924 | 11960 | TCAAAAGCTCTGTCACATCGCGCTGGCAAGACGCTTAATCAAAGTGAGCG |
| | | | | | | 7603 | |
| | | | | | 1925 | 70947 | CGGAGCTCTACATCAGAGGGCATTTAATTGGCCTTGAAATTTTCGATCGA |
| | | | | | | 218 | |
| | | | | | 1926 | 25034 | CGCCCGCTGCTCACTACACTTGTTACCGCTTGTCCTGAGCGCGGAGAGGG |
| | | | | | | 093 | |
| | | | | | 1927 | 33701 | CGGAGCCTGCCTCAGCCGCAGGCTCCATAAACCCAGCCAGGAACCAAGCG |
| | | | | | | 321 | |
| | | | | | 1928 | 53097 | CGGCTGCGGATCAATACCCGCTCACTGTAATCCCAGTAATTGTAGGGCTC |
| | | | | | | 649 | |
| | | | | | 1929 | 51128 | AGAACGTGGGGCTCAGCATCGCCGTGGTAGACTACGACTGGTGAGGGGCG |
| | | | | | | 640 | |
| | | | | | 1930 | 15472 | GGGCTTGCTCCCCTAACCAAGGCGCTTCGGGATCGCGGGGCTGGTGTTCG |
| | | | | | | 9622 | |
| | | | | | 1931 | 16051 | CGGATACTTTCCAGGGAAGGAAATAAGACAGGAACATGTGCCCTAGTGGA |
| | | | | | | 4445 | |
| | | | | | 1932 | 22369 | AGTGTTTCCTCTACATCTCGACCCATTGCTGTTGGGAGCGGCACCTTACG |
| | | | | | | 908 | |
| | | | | | 1933 | 21682 | CGCGGAGTTTCGTTGTGTTGTTACACAAGCCAGCTTTTGGTTTGTTAGTT |
| | | | | | | 362 | |
| | | | | | 1934 | 79620 | CGCCCTCCGCGCCGAGAATGCGCCCTTCTGCCCGGGCTTGATATCACTTT |
| | | | | | | 363 | |
| | | | | | 1935 | 17695 | CGAGCAGAGAAGACATCTTCCTTGGTTTTGCTCTGCGGGGCTCCCCGCAG |
| | | | | | | 25 | |
| | | | | | 1936 | 79611 | CGAGTATGAAGTCGTAATCCCATAGCTGTGAGTTCAAGTCTGGAATTCTT |
| | | | | | | 11 | |
| | | | | | 1937 | 11098 | CGCGGGACCAAAGCTTCCGGCGTGTCCCCAACTTTGTGGCGCCCTCAGGC |
| | | | | | | 6685 | |
| | | | | | 1938 | 14938 | AGCGGCTCCGAAGTGGACAGGTGGCTCCCCAGGGTGCGGAGGCGAGGGCG |
| | | | | | | 9929 | |
| | | | | | 1939 | 10131 | GTTGCATGAACGAGTGGTGTGAAGCCTGTTGGGTAGCTTGGCCACTCCCG |
| | | | | | | 7620 | |
| | | | | | 1940 | 25454 | TGATGGTGTTGCAGGCTGGCCCAGGGACTTGTGGGCTTTGGGCCAGGTCG |
| | | | | | | 202 | |
| | | | | | 1941 | 85416 | CGAGCCCTCTCGGGCCCCGCCAAGTATTTTGTTTGTTCGAAAGTCTTCTC |
| | | | | | | 865 | |
| | | | | | 1942 | 38676 | CGTTGCTTTGCTCGGTAAGATCCCTGCAAGCCTTTATTTGAAACTAGCCT |
| | | | | | | 865 | |
| | | | | | 1943 | 13991 | CGCTGGGTACCTCCTACAAGGCCTTTAGTCCAGGCATGAAGGCAGTTCAG |
| | | | | | | 8866 | |
| | | | | | 1944 | 11138 | CGCTAGGGGGATACATCGACGCCGAGATCCAGAACTGCTTAGCCAAGTTG |
| | | | | | | 5659 | |
| | | | | | 1945 | 10887 | AAACTTGGAACAGCTACCACAAAGCGAACGCCTATCTGTTTGGAATTTCG |
| | | | | | | 023 | |
| | | | | | 1946 | 29975 | CGCGGGTACCAGGGGCCATGGGGAGCCTGCTCGATCTCCTGTAGATCTCC |
| | | | | | | 366 | |
| | | | | | 1947 | 41026 | CGTGTGCTGTAATTCTAAGGAAAACATTAGTAGCCTATTTTTAGCAAGTA |
| | | | | | | 197 | |
| | | | | | 1948 | 12195 | CGCCTCTCGGCTCTCAGCAGCTCCAAGCCCCCGCCACAGCCCTCCGCCAC |
| | | | | | | 6890 | |
| | | | | | 1949 | 28677 | TTGTTTAGAAGAGTGAAACCTTCTGTCTTGCCGTGGGCCTGGTGAAAACG |
| | | | | | | 272 | |
| | | | | | 1950 | 87908 | GGAAACTCTAAAGCGCCGGATAAATAAGCGCTGCAACAGGCAACCACACG |
| | | | | | | 785 | |
| | | | | | 1951 | 91036 | GTATCTTTAGACCTTAGCGCATCCATCTCTCGGTTCTTTCGTCAAGACCG |
| | | | | | | 807 | |
| | | | | | 1952 | 21252 | CGGCTGCTTCACGGGGCAGGAGGGCACCTGGAATCCCCCGCCAGGTCCCT |
| | | | | | | 43 | |
| | | | | | 1953 | 17610 | CGGGGTTCGAGTCGCAGCCTCCAGCCTCAACCCGCACCTCCCGCCCTGCA |
| | | | | | | 7201 | |
| | | | | | 1954 | 55322 | CGCCATTTTGTCAAGGGAAAAAGCAGATGGAGGTCGTGTATATACAAATA |
| | | | | | | 753 | |
| | | | | | 1955 | 32173 | CCCACTGGCAGATGCCTTTGGTAAGCTCGCCAATGGCAGGCTTTCATGCG |
| | | | | | | 206 | |
| | | | | | 1956 | 37268 | GGGATCGCATTTGCTCCTTCTTGGACTCTATGAATCTTATCCTAAGTACG |
| | | | | | | 58 | |
| | | | | | 1957 | 38821 | CGATGCAGCATCTATTGGGGAAGGAATTTAGCTTCTACTGCAGTGTGACT |
| | | | | | | 155 | |
| | | | | | 1958 | 51522 | CGGCGACTCCCAGGTGAAGCTACCTGCACCCCACCCGGGTTGGGGTGGAT |
| | | | | | | 894 | |
| | | | | | 1959 | 49629 | CAAACGCCGCATAGGTGGGAGGGCGGCGGTTTGGAACATTCTGCCATCCG |
| | | | | | | 9 | |
| | | | | | 1960 | 36132 | CCCTACTGGGCCTGGTGGAGGATTGGGTGGGCCTTCACTCCTGCTCCACG |
| | | | | | | 561 | |
| | | | | | 1961 | 55304 | CGGAAAAAACTATCCTTGAGTGAGGCCTGACACTGTGCCTGTGATGATGT |
| | | | | | | 314 | |
| | | | | | 1962 | 26273 | CGGTATCACTTCGGCTGCGAACATGCCTGAACCAGCTAAGTCAGCTCCCG |
| | | | | | | 182 | |
| | | | | | 1963 | 33842 | CGCCGGCGGGACAGGCACTTGGAAAGCAAGATCCAGGGTTCTGAAGATTA |
| | | | | | | 301 | |
| | | | | | 1964 | 31881 | GCGGTCTAAAGTGTTTACTCTGCTTCTTCTCAGAAATGGCTTTGATAGCG |
| | | | | | | 72 | |
| | | | | | 1965 | 57265 | CGGGCCTGGGGGGTCACCCACAGGAAAGGTGAGATTAGCAGCACCATGTG |
| | | | | | | 560 | |
| | | | | | 1966 | 22369 | AGCCTCAGCTGTGTCCTTAGAGGCCTGAAGTCTCTCCTGGCCTCACTCCG |
| | | | | | | 802 | |
| | | | | | 1967 | 69915 | CCCCTAGTGACACAGCCAGTGCCAGAACTAACTTTGACTTTCACTCTTCG |
| | | | | | | 406 | |
| | | | | | 1968 | 96897 | CGCACTACGAGAAGGGGAGTTTGATCTTGTCTCATTGGCGTCTCGGGTCT |
| | | | | | | 414 | |
| | | | | | 1969 | 25890 | CGGAGACAGCGGCAGGGATGCGGCTCTGCTCTACAGGCAGACAGAAACTT |
| | | | | | | 808 | |
| | | | | | 1970 | 10531 | TGGCCAGGGCCCTCATCCAGGAGCCAGGAAACATCTACTACCACGAGTCG |
| | | | | | | 9558 | |
| | | | | | 1971 | 17267 | GGATTCCGAGGATCCGAGGGAGTCGCTTTCAGACTTCCGGTGTCCTTTCG |
| | | | | | | 0879 | |
| | | | | | 1972 | 10190 | CGGTTTGGTATCCATCCGCTGCTCCCTCCAGCGCGGCTCTTCGCGACCTC |
| | | | | | | 6035 | |
| | | | | | 1973 | 61128 | TTGTATCCTAAATCTGGTCCTCAAGATTCTTGAAGTCCCCCAGTCCATCG |
| | | | | | | 572 | |
| | | | | | 1974 | 33700 | CGCGGCAGAGTTCGCGGGCTTAGCAGACCCGCCCATTGGCTCTTGCATCT |
| | | | | | | 759 | |
| | | | | | 1975 | 83961 | TGGAACCGGGAGTCGTGTCAGCACATCCTTGAGAGGCAGGGGTGTGGACG |
| | | | | | | 63 | |
| | | | | | 1976 | 13345 | CGCAGATGAGTTAGAGACACAGAGAAGTGTACATCCTTCAGCCTTTCCCG |
| | | | | | | 3019 | |
| | | | | | 1977 | 91580 | GCTGTGCTCTGTTCCCTTCTGACTTACTGTTTGTTTGGGGCGTCTGGACG |
| | | | | | | 605 | |
| | | | | | 1978 | 46917 | GGAACCGCTGCCTTTCAGGAAAGGTGCTGACGTGGGCCACTGCCGCTTCG |
| | | | | | | 018 | |
| | | | | | 1979 | 14062 | CGGACCAGTGTCTCCGGAGAATGCTATTCTCCTACATTTCCGAACAGGTT |
| | | | | | | 5004 | |
| | | | | | 1980 | 10503 | CGGATGGGAAACCTCAGTCGTTTAGGCACCCCTCCGCTCGAGTCACTTCC |
| | | | | | | 6611 | |
| | | | | | 1981 | 29293 | AGTGGCTAGTGGATCGTGCACCCACCCTCGGAGGCCCGGAGTGGATCCCG |
| | | | | | | 279 | |
| | | | | | 1982 | 14240 | CGGAAGTGAGCTGGATGACAGGGATCAGTTTGCAGCCCACCTTGCCAAAC |
| | | | | | | 9417 | |
| | | | | | 1983 | 79817 | GGCATTACAATACCAGAGGTAACGAATCAATTAAATCCATTTTCCCCTCG |
| | | | | | | 278 | |
| | | | | | 1984 | 46999 | CGCTGTGGCCTGAGACGCTTTTCTTAAAGGTCCCGATGACAAGGACTTGG |
| | | | | | | 274 | |
| | | | | | 1985 | 89951 | AACTCTGAAACCTGAGGGCCTCTGCCGGAAGCTGCTGTTCGGCCCATCCG |
| | | | | | | 787 | |
| | | | | | 1986 | 10542 | AGAGAGAACGGTTAGGCAGGCAGGAGGTCCACAGTGAGTGGCTGCAGGCG |
| | | | | | | 8506 | |
| | | | | | 1987 | 76102 | CGACAGAGTCCACGACGATGGCCGAGGGAACAACACTGTCCCGTCCCAGA |
| | | | | | | 450 | |
| | | | | | 1988 | 36919 | TAAAGAGTAAAATCACGCGCAATCCATCCCAATATAACCGCAGTTGTTCG |
| | | | | | | 905 | |
| | | | | | 1989 | 15302 | AAACCCCTGGTACCTGAGCACTGATCTGCCTTGGAGAACCTGGTGAGTCG |
| | | | | | | 9939 | |
| | | | | | 1990 | 13702 | CGGATTTGAACCAAGAGGCCATGAGATGGTACCTTGTTTTGTTTTTAGCA |
| | | | | | | 8617 | |
| | | | | | 1991 | 10089 | GCTCTCTGCGAGAGTGCGGGTGGCGGCACGGCCGGGTTAGACAAGGCTCG |
| | | | | | | 5050 | |
| | | | | | 1992 | 15717 | AGAGAGATATGTAAGGCCCAGGGAGCCGATGGCGCCCTGCAGTGACAGCG |
| | | | | | | 8731 | |
| | | | | | 1993 | 11061 | GCTCACTCGTGTGGACTGGAAACGCTCCGAGCCGGTTATTTTAAAAACCG |
| | | | | | | 0899 | |
| | | | | | 1994 | 16216 | TGCAAAGCCTGGGAACAGTGAGGGCATTAGGACCAGGAGTGACGCGCACG |
| | | | | | | 100 | |
| | | | | | 1995 | 17783 | TGGGGGGTTTGCGGTGGGCGGGCACTGCGCTCTTGGACACGTGGGTGGCG |
| | | | | | | 331 | |
| | | | | | 1996 | 19146 | ACCCAACGGAGGGTTTATTCTGCCCAGCTTCACATCTGGGGGTCAAATCG |
| | | | | | | 324 | |
| | | | | | 1997 | 21525 | CGCCCCGGCGTCTTTGTGCGCGAGCCTCGTTCAGCCCCTCTTCGCATGTC |
| | | | | | | 6262 | |
| | | | | | 1998 | 10510 | CGCCTGGGGCCTGAAGCTCGTGGAATCACCCGCCTCACGTGTCTTTGCGG |
| | | | | | | 3075 | |
| | | | | | 1999 | 23716 | ACGAGAAGGTGCCACTCAAACCTGAGTCAAGGAACACAGCTAAGTCCTCG |
| | | | | | | 8540 | |
| | | | | | 2000 | 23834 | AGGGGTGCCTGCGTGCTGGTTGCTGCTGCCCGGGGCGAGGCGCCGTAGCG |
| | | | | | | 977 | |
| | | | | | 2001 | 91957 | GCGTCGGCTGTGATGGGGAGCCCGGAGCAGCCAGGCGGCGAGCAGTAGCG |
| | | | | | | 989 | |
| | | | | | 2002 | 48145 | AAGGGGTTCCCCGCTTGAGCTAACGTAGTGGTGGCATCCAGAGGTGAACG |
| | | | | | | 000 | |
| | | | | | 2003 | 43276 | CGGGCCTCACCGAAGAAAACAGGTTGCTGGCAACGCGGACAGGTCCAGCT |
| | | | | | | 478 | |
| | | | | | 2004 | 11052 | CGGCCAGGAGACCCTCCCTGTGCAGGCAATGCCGGGTGAGGCATCGCTCC |
| | | | | | | 2265 | |
| | | | | | 2005 | 52276 | TTACTCGAGAATCAAAATAAAGAGGCGAGCCAGCGTTCCAGAAAATCACG |
| | | | | | | 947 | |
| | | | | | 2006 | 50792 | CGGAATTACCTGATTTGTGCCTGGATTTACTGATAGTAATAAGAGTGATG |
| | | | | | | 868 | |
| | | | | | 2007 | 76059 | CGGGGTCAGCCTCCTGCCAGCTCTCATAAGTAGATCTAATTATGACCGCT |
| | | | | | | 576 | |
| | | | | | 2008 | 87974 | GGTGGCTCTTGGTGCAGGGCATGCTGCTCTCGGAGGAGTTGGGGTCTGCG |
| | | | | | | 446 | |
| | | | | | 2009 | 97499 | CGGGCCTGGGTTTAGGGTCCACAGCTCTTGCCAAATTCCAGAGGCTGGAA |
| | | | | | | 987 | |
| | | | | | 2010 | 29795 | CGCCCCCAGGCTCCCACTCCATGAGGTATTTCAGCGCCGCCGTGTCCCGG |
| | | | | | | 815 | |
| | | | | | 2011 | 14143 | CGAAGACCTGTATTACAGGTAGTCACTTGTCTGTATTAATACTGAGATGT |
| | | | | | | 8973 | |
| | | | | | 2012 | 32455 | CGCTGTGCTTAGTCAGGATACAAGGAGGGAACTCGGCACACGATAAGTAT |
| | | | | | | 362 | |
| | | | | | 2013 | 34819 | CTCACTGGAGGGGACGGGTCTGGAGCAGGCAGAGTGAGCCGCTGCTTGCG |
| | | | | | | 740 | |
| | | | | | 2014 | 97646 | TTTGGAACCTCTGCAAACACTTTCTCCTGGGTCAGTTTCCCGCCAAACCG |
| | | | | | | 803 | |
| | | | | | 2015 | 13325 | TGTTTTCCCTCTGCGATGTCCTCTTTTAATTCACTAGGTCAGGAAAAGCG |
| | | | | | | 6591 | |
| | | | | | 2016 | 13650 | CAGGATCCGGAGGCAGCGCCCTGTGCAACTTTGGCAACTCGGAGGACGCG |
| | | | | | | 9451 | |
| | | | | | 2017 | 27339 | CAGTATTCGCACACTGATAAGAACACCTCCCCATAATCCCCTGGCCAGCG |
| | | | | | | 4 | |
| | | | | | 2018 | 39864 | CGGCGTGTGAGCTTTGTCCTTTGGTGGGGCTCCTGGCCACGGGCCGAACG |
| | | | | | | 984 | |
| | | | | | 2019 | 57976 | CAGGGGACTGGGGACCGAGCAGGAGCTGGCGGGGTGAGCTCTGCGAAGCG |
| | | | | | | 756 | |
| | | | | | 2020 | 22316 | CGCTCCACTCAGGCCTGGACAGCCAGCCCGCTGTTATTTTCACCTGTAGT |
| | | | | | | 6909 | |
| | | | | | 2021 | 47425 | ATGGAGGGAAGTCACTGGTCACACAATACCCTTTGGGTCGGTGTGAATCG |
| | | | | | | 198 | |
| | | | | | 2022 | 17825 | TGGGCGGAAGGCATCCCGGTCCCTGAAAGTAGGGACCCACCTCTCTTTCG |
| | | | | | | 7923 | |
| | | | | | 2023 | 10184 | ACCTCGCAGGAGGTGACAGGAGGACGCTGGGTCGAATCACAGTCCTAGCG |
| | | | | | | 9938 | |
| | | | | | 2024 | 96898 | TTGTGACATAGTTGTAATACAAACTCAGCCGCCTTGCACTGCATCTTGCG |
| | | | | | | 476 | |
| | | | | | 2025 | 30292 | CGCAAGTAGTATGACATATAACAGGGCTTGGCAAATCTTTGCTGTAGAGA |
| | | | | | | 899 | |
| | | | | | 2026 | 12844 | CAAGAGCTCCTAATTCGGTGACACTACACCACAATATGTGTGATAAGTCG |
| | | | | | | 6370 | |
| | | | | | 2027 | 11489 | CGAAAACACAAATAGTAAGTGGTAAGAAGGGCTGAAGTTACTACCTACTG |
| | | | | | | 1274 | |
| | | | | | 2028 | 26250 | CACAAGTCCTATCACACTACATAATTCAGGGCGCGAAACTGGAAGATTCG |
| | | | | | | 919 | |
| | | | | | 2029 | 10831 | CGTGGAGGTGGAGCAGGACAACAAGATGACCCCCGGGAACCTGGGCATCG |
| | | | | | | 93 | |
| | | | | | 2030 | 91580 | CGGCAGCTAGCCCGCCCCAGTTCTTAAAGCTGCAGAGCTGCTGGCAGCTG |
| | | | | | | 380 | |
| | | | | | 2031 | 57646 | TTGAAGGTAGCGAGGACTTATTATCACAGTATCCTTTGAAAACCCATTCG |
| | | | | | | 138 | |
| | | | | | 2032 | 10091 | CGCTTTGGGGACTGCGTTTTATCCACGATAAACCATTTCCCGAGAAAGGG |
| | | | | | | 3948 | |
| | | | | | 2033 | 20033 | GCAGGAGTAGTGAGCGATGCCGGGGCTGGCTTTCCTAGATTTAGGAACCG |
| | | | | | | 4137 | |
| | | | | | 2034 | 36105 | GGCTCTCACCTGAACCCCGAAGCGTAGTGTCTTCTCTCTGGACTAAAGCG |
| | | | | | | 364 | |
| | | | | | 2035 | 50889 | AGTTTCCGTGCGCCCGAGAGCGTGTGAATTGCTCCTAATTACCGTGTCCG |
| | | | | | | 427 | |
| | | | | | 2036 | 16024 | CGCGTGGCAGGCTCCCAGAGGAGAGGGCCCTGTTATTTACGTCTTTGTCA |
| | | | | | | 81 | |
| | | | | | 2037 | 38892 | TTCTTACCCCCTGTATCAAAGAGTTAAAATCTAATTTTTTCCCAAGATCG |
| | | | | | | 846 | |
| | | | | | 2038 | 48397 | CGATAACATTCCAACCAGACCGTGCTTTTCAAATGCCCCCGAAAATAGCG |
| | | | | | | 225 | |
| | | | | | 2039 | 13959 | CGGGTCTGCCTGAGGATAGGCATCCTCCATACCTGGCAAGCAGCAAGCGG |
| | | | | | | 3443 | |
| | | | | | 2040 | 20865 | TTTCCTTTAAAATTGCATTTTCTGCAGAACTCAAAATCTAGACTGGGTCG |
| | | | | | | 674 | |
| | | | | | 2041 | 14266 | CCGATGCAGGCTCAGATATGCTGCCTGCGGGATGTCGTGCTTTCCAGGCG |
| | | | | | | 6476 | |
| | | | | | 2042 | 67070 | GTAGAAGAAAGCGCCTCATTCTTTTCCAGGCATATAAGGACGAACCCACG |
| | | | | | | 120 | |
| | | | | | 2043 | 59612 | CGACTTCTTTCTACAGTTACTAAGCGCCCACTGTGTGCCCTGGAAGGGAC |
| | | | | | | 408 | |
| | | | | | 2044 | 18771 | TTCAGAGGCCTTATAGCCTCGGCGAAGCATGCTGGGTGCGGGCGGAGGCG |
| | | | | | | 3876 | |
| | | | | | 2045 | 58145 | GTGGCTTTACTGAGGCGACTGGAGGCTTTTGAGCATCCCAATGTTGTCCG |
| | | | | | | 283 | |
| | | | | | 2046 | 12404 | CGAGGGACTGTTAACCATGACCGTCCTCATAGATTAAATTAGGTACTTGC |
| | | | | | | 3067 | |
| | | | | | 2047 | 22370 | CGCCGCGACAGAGGGGAAGCAATTGTACCCTAAAGTCTACACTGTCTGTC |
| | | | | | | 046 | |
| | | | | | 2048 | 38067 | CAAGTGGGGGATCCGCGGCAGTGAGGACTGTAGGGTGCGCGGGTACTCCG |
| | | | | | | 622 | |
| | | | | | 2049 | 28129 | TGAGGTAGGAAGTCTGAGCATTGCCGACGGGCCGGAGCAAATCGCTCCCG |
| | | | | | | 416 | |
| | | | | | 2050 | 81106 | GGTTGTGATTAATTTTTCCGAGGGGATTTGGGCCGGGAGCTTACGGAACG |
| | | | | | | 312 | |
| | | | | | 2051 | 36728 | CGCCCCCCGCCCAACCCGAGGCGGCGATCCCGGCCCCCACAGTCGCTCCC |
| | | | | | | 859 | |
| | | | | | 2052 | 50444 | AGTGCCGGTGAATAATTCATGGGGCTGGGAGGCCCTTATCTCCCTGGCCG |
| | | | | | | 723 | |
| | | | | | 2053 | 19499 | CGCTGCCAGAGTATTTGTGGGAAGAGAAAGAGGAGGTTGGATGAATCACT |
| | | | | | | 118 | |
| | | | | | 2054 | 11637 | CGCTCCCCCCACGAGCCGCCGCACTCCCTAAATGAATGAACTGGCTGCTC |
| | | | | | | 1330 | |
| | | | | | 2055 | 14541 | CGGCCTCTGTCGAGCCCTCCGCTCCTATGCGCTCTGCACTCGGCGCACCG |
| | | | | | | 5412 | |
| | | | | | 2056 | 42145 | CGTCTCTTGGGGCTTGCCGAGATGGGCGGAGCCTTGGGTTATGATGGGCG |
| | | | | | | 954 | |
| | | | | | 2057 | 30405 | ACTGACTGGTTGCATCTTGGCCAACTGCTCCTAGGCAGGGCTGCATTTCG |
| | | | | | | 328 | |
| | | | | | 2058 | 48144 | CGGGGACCTTCGCGAGTTACTTTGTAAAGGCGAATCCAGGCAAGAGAGCC |
| | | | | | | 369 | |
| | | | | | 2059 | 20512 | CGGCCTCCGCCCTCCTGGCCCAGCGGCCCCCACCCGACCCCACCCGACCC |
| | | | | | | 463 | |
| | | | | | 2060 | 10848 | ATGCTTAAATTTCCACTGTTGGACGAATTCTGAGCGCCCAGGGAGCAGCG |
| | | | | | | 7820 | |
| | | | | | 2061 | 11840 | CGCCCTCCGGCACACACTGCTGCTCCTGCCAGCCCTTCTGAGCTCAGGTA |
| | | | | | | 2540 | |
| | | | | | 2062 | 10542 | AGGGAGGATCGTGTGGGATTCTCTGTGCTCCGAAGGAGTCCTGCATTTCG |
| | | | | | | 8651 | |
| | | | | | 2063 | 20134 | CGCTCGAGGACCTTTGCCCTGGGCCGCCGGGCCCGCCCCCCCACAGGAAC |
| | | | | | | 778 | |
| | | | | | 2064 | 13273 | GCTGACCACCAGGAATAAACAGATTCCTGGCAATAGCCATTATTAGATCG |
| | | | | | | 5814 | |
| | | | | | 2065 | 17444 | CGAAGGGCCCCGAAGGCAACCCGGTGGGTAGGGGCGCGGCGGACAGGAC |
| | | | | | | 0103 | G |
| | | | | | 2066 | 13546 | GACCTCTGGGGCCCCGGGAACAGGACATGAACCCTGAGGGGAACAGGACG |
| | | | | | | 2522 | |
| | | | | | 2067 | 46951 | CAGGAGATGGCTCGTGGCAAATTGCTTTGTTAGTCTTGTCAGCGCGGCCG |
| | | | | | | 354 | |
| | | | | | 2068 | 50892 | GAGGCATTGACTTTTGCGCTGTTTGCCCGCGGCCTCGGAGCAAGCAGACG |
| | | | | | | 674 | |
| | | | | | 2069 | 91184 | TACACTCCAACGAGGCGTTACAGTCATGCTCCGGGTCCCAAGTGGATCCG |
| | | | | | | 501 | |
| | | | | | 2070 | 50373 | CGCCAAAATCTTAGCCCAGAGCTTGGGTGCAAGGGTTGAGTTGAGTGTAG |
| | | | | | | 641 | |
| | | | | | 2071 | 11930 | AACAAATAATTTAAAAATAAATCTCCATGAAAACTCTGGAGAGCAGCCCG |
| | | | | | | 4705 | |
| | | | | | 2072 | 26273 | CGAAGTGATACCGTCAAATGACCACAGGAAAATGCTGGTGAGCCCAAACG |
| | | | | | | 145 | |
| | | | | | 2073 | 13663 | GCGGGATGAGCTGCTTTCCAAAAAGGATCTCGGTGATTGGACCGAGCTCG |
| | | | | | | 2433 | |
| | | | | | 2074 | 54609 | TGGCTCGGCGGATCAGATCGCTCGGTGCCGAGGAGGACAATACAGTGACG |
| | | | | | | 776 | |
| | | | | | 2075 | 95239 | CGCAAATTGTCCACCTCGTGTTGCTAAGCGATTGTTTGTCGGCCCCGCAC |
| | | | | | | 570 | |
| | | | | | 2076 | 73846 | CGGGCCCATCCCCCACAGCACCCCCAACGATCTCGGGGTCCAGCCACACC |
| | | | | | | 858 | |
| | | | | | 2077 | 11513 | CGCGCCCCAAGAGCCTCTGCCAGGCCGCTGGGACTTGATTCATTTCTTGA |
| | | | | | | 6200 | |
| | | | | | 2078 | 26047 | CAGCCTGTAGAAATCCGTGCTTTGCTTCCTGCACGGCGGGTAGAGCAGCG |
| | | | | | | 996 | |
| | | | | | 2079 | 28998 | CGGACGCAGCCCGCGTGCTCCCCGAGCTATTGCACTGGAGCCGCCCTCCC |
| | | | | | | 195 | |
| | | | | | 2080 | 15112 | CGGCTTTGCAACAGGCATTACTCGCCATTGATTGGTGCCCACCCAGGGCA |
| | | | | | | 2266 | |
| | | | | | 2081 | 10091 | GGCCGAGGCCGCGGCTGTCGGGGTGGCCTGGACGGGTGCAGGGCTGGGCG |
| | | | | | | 3842 | |
| | | | | | 2082 | 48546 | AGAGCGGGGGGCTACAGCCGGGGCTCAGGGCTCGGGGCTCGGGGGCCACG |
| | | | | | | 431 | |
| | | | | | 2083 | 88877 | TCTCCCGATCCAACTCACGAGAGATAAAGATGTCTAAATAACCACGACCG |
| | | | | | | 216 | |
| | | | | | 2084 | 12230 | CGCGGGAGCAGTTCATCGCTCTGTACCGGCTGCGACCGCTCGTCTACACC |
| | | | | | | 1740 | |
| | | | | | 2085 | 33718 | AACCCTGGCAAGCCACAGCTTCCTGCAGTGTCCTGTTGCCATGGGTTACG |
| | | | | | | 76 | |
| | | | | | 2086 | 38807 | TAAAATCCGCCTGCCCGCAGTGAGTGGGAACTGCTGCAGATCATTAAACG |
| | | | | | | 712 | |
| | | | | | 2087 | 24641 | CGGAGCCCATTGGTCGGAACACCTCACAATGGACCCCAGCGGCGCGCAAA |
| | | | | | | 077 | |
| | | | | | 2088 | 55610 | CGGCCCACTCCTCTTGTTAAAAGTAAAACCAGGCCGGGCGCGGTGGCTCA |
| | | | | | | 624 | |
| | | | | | 2089 | 20849 | CCTCGAAGCCACAGGCCCCACCTTCCGATGGAAGATTAATCTGGAGGCCG |
| | | | | | | 2057 | |
| | | | | | 2090 | 36978 | GCCATTTGAGCGTGTTTCAGCCAGATAATTACGTTGCCATTGTGTTACCG |
| | | | | | | 633 | |
| | | | | | 2091 | 16283 | CGCTTTTCAGCTCCTCAGGTGATAACAGAAGCCAAACTCCAAGATAGTGG |
| | | | | | | 1837 | |
| | | | | | 2092 | 91192 | TCACTCCATGCCATGAGAGAGAACCTGGCAGTGTCAGATTCACTCCAGCG |
| | | | | | | 803 | |
| | | | | | 2093 | 12721 | CCTGGCACAAAGGGCCACGGGCGCGAGGGACTCGATGAGCTGGGAGATCG |
| | | | | | | 1421 | |
| | | | | | 2094 | 10068 | CGTGGTTGCGTAAGCACATCTAAAGGTTAAACTCCAAGCAGCTAATCTGC |
| | | | | | | 6693 | |
| | | | | | 2095 | 62356 | CGCATATCCGCATCCACGCGGGCTACAAGCCCTTCGTCTGCGAATTTTGC |
| | | | | | | 962 | |
| | | | | | 2096 | 51138 | AGCCATGAACATAAATCCGGCCAAGTCGTGGCCCCGCAGCGTGGGCTGCG |
| | | | | | | 790 | |
| | | | | | 2097 | 29186 | AGCGCTGACTTGATGCGGCCAGGTTTCTGGGACCAGGAGGTTGGGACACG |
| | | | | | | 234 | |
| | | | | | 2098 | 67070 | CGGCCAGGTTTCAAGGGCTTTTCTTGGGTAGAAGAAAGCGCCTCATTCTT |
| | | | | | | 045 | |
| | | | | | 2099 | 53092 | CGCCTTCTAAAGCCTCGCCCAGCGCCGCCGAAGCAGCTTCACCTCTCCAA |
| | | | | | | 334 | |
| | | | | | 2100 | 10289 | CGCCCCCCTCCCAGAGGCGTCCCGCACCTGTCTGAACTGTTAAGAAATCT |
| | | | | | | 6869 | |
| | | | | | 2101 | 13972 | AATTACACTTTACCGTAGTCTCTATCAAATGGGAGCCCAGAGGAACTCCG |
| | | | | | | 099 | |
| | | | | | 2102 | 13536 | TGTTTCGGAAAGTCCAGGTTTTCCTCGGGGAGGACTTCAAACTCACTGCG |
| | | | | | | 910 | |
| | | | | | 2103 | 11840 | CGGATCCTGGAGTAGAGACGAACGGTTCCTGAAGCTCCCGGTAAATGCCC |
| | | | | | | 7296 | |
| | | | | | 2104 | 35395 | ACAGGGAAGGTGCCTGGGGAGGGGCCGGGCGGTGCTGTCTCCTGACTCCG |
| | | | | | | 800 | |
| | | | | | 2105 | 21022 | ACTTAGGGGTTCAAATGCTGCATTACCGCGCTCGGGTGAGCGACTGCCCG |
| | | | | | | 587 | |
| | | | | | 2106 | 15880 | CGCTAACAAGCATTGGAATCGCAGGCTCAGAGCCAGATGGCCTTAGAGAC |
| | | | | | | 0888 | |
| | | | | | 2107 | 28367 | AGGGATTTCCCTGTCTTAAGATCAGTTCTACAAGCCTCGACCACCCATCG |
| | | | | | | 749 | |
| | | | | | 2108 | 43423 | CGCCACCCCGCTCAGTCTCAGCGGGACACCTCCACAGCCAGTAGGAAGGG |
| | | | | | | 072 | |
| | | | | | 2109 | 72897 | GCGAAAAGTAGCTGGAGCCGGAGCAGGGACAGAACCTGTTGCTGCAGACG |
| | | | | | | 868 | |
| | | | | | 2110 | 86048 | CGGCCGTCCACGCAGGAACCGCAGTACTTGGGCCGGTATTTCTTCACACT |
| | | | | | | 540 | |
| | | | | | 2111 | 10844 | CGGGGCCACTTTGACCGCCAATAGTGGGGAAGTCAGAGCTAATCCTCAGA |
| | | | | | | 0280 | |
| | | | | | 2112 | 54422 | AGCAGGTTCTGCGGTAGAGAGCGCAGTCCTGCTGGCCTCTGGAGAGTGCG |
| | | | | | | 775 | |
| | | | | | 2113 | 39170 | CGGGAATCTGAGCCGTGCCAATCGACGTGGGGAAAATTTCACAGCTGCTG |
| | | | | | | 763 | |
| | | | | | 2114 | 13356 | CGTGAAATAAAAACCAACCCAGACCAGACTGGGGCGCTTTTTCTGACGGG |
| | | | | | | 1932 | |
| | | | | | 2115 | 39015 | CGCCGGGGTGCGCTCCAGGCTTGGCTGCAAACCCCCCAAGAATTAGAAAG |
| | | | | | | 705 | |
| | | | | | 2116 | 54391 | CACTTGGGCGGCTGCTGGCAGCGATGAGCAGAGACGTTTAGCCCTCAGCG |
| | | | | | | 631 | |
| | | | | | 2117 | 16986 | CTAACCACTCTGCCTAAGCAGCTATGACAGTGCATCCCACAGTTAAAACG |
| | | | | | | 186 | |
| | | | | | 2118 | 58216 | GGGCCTTGAAAGCGAACTCAGGAGATGGGGCGTTGTGTATAGGCAAGTCG |
| | | | | | | 297 | |
| | | | | | 2119 | 97646 | CGGGACCTCTGGACATCCTCCATCCCGGTTTGGCGGGAAACTGACCCAGG |
| | | | | | | 778 | |
| | | | | | 2120 | 47703 | CGACAGGACCATTCTGGAGAACCATTTTGGCAAAATTGCTAAAGTGCAGC |
| | | | | | | 630 | |
| | | | | | 2121 | 49736 | AGTTGGTTATGATGTTTTTTCCCCTACTCAGCTCTAAAAAAACATCCTCG |
| | | | | | | 085 | |
| | | | | | 2122 | 26190 | CTTCCTCGCCTGCGGGTGGTGCGTTGGGAAGGCATGGGGTGCGGAACCCG |
| | | | | | | 347 | |
| | | | | | 2123 | 47717 | GGGCCTCTTCGGAGGGAGCTGAGGGCCGCGTTCCTTCTGAAAGCGGGACG |
| | | | | | | 575 | |
| | | | | | 2124 | 25055 | CCATGGGGAGGCTGCGGGGACGCGGGGCTGCAGAGAGCGGCAGTGGCAC |
| | | | | | | 967 | G |
| | | | | | 2125 | 26527 | CGACCCGCTCGTCGACGAGCGCCTGTTCAACACCTCTGACTCCTGCTCCT |
| | | | | | | 928 | |
| | | | | | 2126 | 20212 | CGCCGGACTGGGTAAAGCCAGCCCTGTAGCTGAAATGTAGGTAGATTGTT |
| | | | | | | 2474 | |
| | | | | | 2127 | 84763 | CGGACTTCCTTGAAATGACAAACAAGTAAATATTTAAGGGAATAAGTAAA |
| | | | | | | 338 | |
| | | | | | 2128 | 17828 | TAGTTTGACAAGTAAGATACCACAAGTCTGTCTGTCACTAAGAGCAAACG |
| | | | | | | 8359 | |
| | | | | | 2129 | 10710 | CGCGGTCCCCAACACCAGACTCCCGCAGCCACCGCGCCCGGTCCCGCCCT |
| | | | | | | 3716 | |
| | | | | | 2130 | 15079 | ATAAAGCACATTCAGAGACTGGCTAGAAGTCTTAAGCTTGGTCGTGCACG |
| | | | | | | 7951 | |
| | | | | | 2131 | 17013 | GCAGGTGGTGGGCTTCGTCACGAGCTTCGTGGGCTGGATCGGGGTCATCG |
| | | | | | | 6920 | |
| | | | | | 2132 | 68178 | CGAAAACAATTCCTCATGAGGAGTGAGCTGAGAGCAGAAATCCAGTGGAT |
| | | | | | | 729 | |
| | | | | | 2133 | 19692 | TCATTCAGCCTTAGGGGGGCATTTCTGCATTCTTTTCCAACAAGATGACG |
| | | | | | | 011 | |
| | | | | | 2134 | 31496 | TCTACGGGCTCGGCAAGAGGTGACGTAAGCGGAATGGGCTCCCAGGAGCG |
| | | | | | | 73 | |
| | | | | | 2135 | 37321 | CGCCCCGGGCCTTTCAGACACTTTTCTGGAATGTGAAGGGAGGTGGGGGC |
| | | | | | | 631 | |
| | | | | | 2136 | 79710 | TACAATTACGTGCAGTACATCATAAAATTCCAACAATATGTAACTCTTCG |
| | | | | | | 803 | |
| | | | | | 2137 | 57090 | CGCGCACACCCGCGCAGCGCGCACAGCCCCCTGGTGCAGCTCCTGCCATT |
| | | | | | | 013 | |
| | | | | | 2138 | 31020 | AGGAGTGGAGACGAGTCAACGCCCCTTCGTACAGCAGTGAGGTTAGAACG |
| | | | | | | 973 | |
| | | | | | 2139 | 70151 | CGCAGTCTCAGGCCCCACGCAATCCTTTTGCAAAGCCAAGAAGTGTGTGT |
| | | | | | | 134 | |
| | | | | | 2140 | 46526 | CGCAGGAGGAGCTGACACTTCACACATTTGTCAAGATTACTGTCAGGCCC |
| | | | | | | 843 | |
| | | | | | 2141 | 12920 | CGCCTTCCATGGGCACGCGGCCCCAGGACGGCCCTCTATTAAAGGATCCT |
| | | | | | | 4206 | |
| | | | | | 2142 | 22873 | CGCCACCAACCGCCCGCGGAGCCAGACCTCCCAGCTCTGCGCCCCAGGAC |
| | | | | | | 6258 | |
| | | | | | 2143 | 74707 | ACGGGGGACCTGGCCGGAGTTGACAAGGGGCAACTTTGTACGTCCACCCG |
| | | | | | | 145 | |
| | | | | | 2144 | 15319 | TTATTTCCAACTCTTCCAAGATGCAGACTCCCTAATGGTGGGTCACATCG |
| | | | | | | 2843 | |
| | | | | | 2145 | 64883 | CGGCTCCACCACCCCAGGTGGGGGCATGTGCCCACTCATCCACCAGCACA |
| | | | | | | 528 | |
| | | | | | 2146 | 36919 | CGCAAATTCGTTAAATTATGATACAATAAGGCCAACCTCGAGGATCCTCG |
| | | | | | | 674 | |
| | | | | | 2147 | 14589 | CGCTCTAGCATGCGTCCGAGCTCTGTTGCTTTTTACAGCCGCCTGGTCCC |
| | | | | | | 5604 | |
| | | | | | 2148 | 12920 | CGGTCTCAGAGGGAAGGAGCGCTCTAGGAAAGGAGGATCCTTTAATAGAG |
| | | | | | | 4125 | |
| | | | | | 2149 | 19788 | CGCCAGGACCCGGCGGGTGTCATGTGTAGGGGGAAATCAAATAAAACATA |
| | | | | | | 7714 | |
| | | | | | 2150 | 14572 | CGCTGCGCCCTGCCGCAATCAGTGGACAGCCGAATACTTCATTCGTTTCT |
| | | | | | | 0024 | |
| | | | | | 2151 | 10547 | CGCCCTGAGTTGAGGTATCCACCCAGGCTTCGGAGTCATTGGAAGGGAAA |
| | | | | | | 9420 | |
| | | | | | 2152 | 83032 | GAGGTGCGCGCTCTGTACGAGGAGACGGAGCAGCTTCGGGAGCAGAGCCG |
| | | | | | | 0 | |
| | | | | | 2153 | 19692 | CAGAAATGCCCCCCTAAGGCTGAATGAGCACCACTTCCACACTCAGGGCG |
| | | | | | | 082 | |
| | | | | | 2154 | 15747 | CCGGTCTTGACTGGAAAAGCTGGTCATTAATTAGCCTCCTTTTGCCTTCG |
| | | | | | | 6887 | |
| | | | | | 2155 | 76859 | GGCGAGGTGGGCGGGGCGGGGCCTGAGGGGGCCTGGACGGGCACCGGGC |
| | | | | | | 010 | G |
| | | | | | 2156 | 15065 | GAGGGGGCCGCTGGCTGGGCACCGGCGGGGCGGGGGAGCCTAGTGTGGCG |
| | | | | | | 2948 | |
| | | | | | 2157 | 11220 | AGCAATGGAAGCAGCCGAGCGTGGCCTCGCCCATGGTGAACTGGAGAACG |
| | | | | | | 5368 | |
| | | | | | 2158 | 38035 | CGTCCAGCGACCTGCACTACTGGGTCGGGAAGCAGGCGGGTGCGGAAGCG |
| | | | | | | 831 | |
| | | | | | 2159 | 41875 | CGGCCACTGAAAGCCTCCCTGTTCCTGGGCAGGGCAGCCAGAGTGCCCTC |
| | | | | | | 606 | |
| | | | | | 2160 | 13100 | CGGACACCTGTAAGTGTGATTTACAAGACTGAGGGCAAATGCCAGGAGCC |
| | | | | | | 2896 | |
| | | | | | 2161 | 36979 | ACATAGGCATATCCAGTTCCTTAGACATTGACTGGGTCTCCTGGGTTGCG |
| | | | | | | 758 | |
| | | | | | 2162 | 11551 | GCTGGCGTGGCCCTCAGGGATCCTTTCTTTTCTTCCCCCAGAACATTGCG |
| | | | | | | 964 | |
| | | | | | 2163 | 10184 | CGCCACACACGGTCGGCTCTTCCGGCGCCAACATGACCCTGGCTTGTTCC |
| | | | | | | 584 | |
| | | | | | 2164 | 36991 | TGCTTAGCCACCACAGTGATCAATGCTCAGTTCGTCGGGGCCTGAGAGCG |
| | | | | | | 034 | |
| | | | | | 2165 | 60386 | CGGGGTTCGTCCCGGCTGTCACGCCACCTTGTGACATCACAGGGCCATGC |
| | | | | | | 396 | |
| | | | | | 2166 | 14710 | CGCTCGCCAGTAATAATATTTCATTACGCTGCTCCAGAGGCTTCCTGGAG |
| | | | | | | 9784 | |
| | | | | | 2167 | 20898 | CGTCCGCAGAGATACCTCTGTGCCCTGGGATTCCCTCATCCCGCTCAGGA |
| | | | | | | 781 | |
| | | | | | 2168 | 17357 | TTTTAATTTCCTTTTCTTTCTGTGCTTTGCAGAAAGGGCTTGGCCTGGCG |
| | | | | | | 587 | |
| | | | | | 2169 | 65409 | CGCGGGATCAGGACGTGGGTTGCCTCCCCGACACCTTGTATGCATCCTGA |
| | | | | | | 861 | |
| | | | | | 2170 | 18067 | CGCCTGCTCCCAGAGACTTGCACGCCTCAATCTCTGCGTGTATCTGACTG |
| | | | | | | 927 | |
| | | | | | 2171 | 67250 | CGGAAATGAGTCATAGTTGTGCCCAATTAATATTAGGGAATAATCGATGA |
| | | | | | | 046 | |
| | | | | | 2172 | 44340 | TCCCTGCCAGTCACAAGCCTCGGGATTATGCGATCAGAATGTGCGCTCCG |
| | | | | | | 839 | |
| | | | | | 2173 | 39649 | CGGGGCTCTCAAGCCCAGTAGGCTCCAATCCGTTGAGTTCCTGTTTTGAT |
| | | | | | | 254 | |
| | | | | | 2174 | 43276 | CAAGTGGAGTGGTTCTAAATTTCTGATAACCAATGAGCGCCGAGGAACCG |
| | | | | | | 647 | |
| | | | | | 2175 | 22032 | AAAGGCAGATCCCGCGGCTTGGGAGTGAGCAGAGTGGCTAAATCCAGACG |
| | | | | | | 5369 | |
| | | | | | 2176 | 17330 | ATGAATTCCCGGTGCTGATGTCCAGAAGAACAATTTCATAACCCTGTTCG |
| | | | | | | 2303 | |
| | | | | | 2177 | 64035 | CGGTAGTCAAGGGAAAATCCTGACTCAGGGTGTGGGGTTCATGAATGAGA |
| | | | | | | 529 | |
| | | | | | 2178 | 12645 | TGCCAGCCCAGAGTCCTTAGTCGGATAATGTATCACAGATACAACAGTCG |
| | | | | | | 0475 | |
| | | | | | 2179 | 46262 | GGAGCTCGGCCCCGCGCGGGGCAGAGGGAGTGGTCCAGGGCTCGGGAGCG |
| | | | | | | 436 | |
| | | | | | 2180 | 58216 | CAAGCGCGGCCTCGGAACCGAATGGACAAAGACGACGGGGGCTGGAGAC |
| | | | | | | 415 | G |
| | | | | | 2181 | 13839 | CGGTGGCAGCAGAGACAGGCAGAGCGCAGTTCCCGCCCACCCCTGCTCCC |
| | | | | | | 3651 | |
| | | | | | 2182 | 73163 | ATCTGGTGCATCTGCTCCCCACGAGCGCGGGCACCTGGGGCGAGAGAGCG |
| | | | | | | 868 | |
| | | | | | 2183 | 22929 | GGAAGAGGTTTGTCCTGGTACTTCGCTCAGCGTTACTTTCAAGAAAGCCG |
| | | | | | | 205 | |
| | | | | | 2184 | 27521 | CGGGTCAATTCAGAAACCAGCTCCCCTGGGGGCAAGTTTGGCTCTGTGCT |
| | | | | | | 385 | |
| | | | | | 2185 | 36044 | GCTGTCATCACTGATTGTTCTGAAACTGCTTTCCTCCACAGCATCTTACG |
| | | | | | | 151 | |
| | | | | | 2186 | 41363 | CGCTGCCAGCTCTTCAAACGTTACCCGAGGGCCCGGACGCTGCTTCCGCA |
| | | | | | | 774 | |
| | | | | | 2187 | 13929 | CATGTGACGAGATGAGACGTGGATGCTCCCCTGTCCTCGGCAGCCAGTCG |
| | | | | | | 0292 | |
| | | | | | 2188 | 60288 | AAGGTAGGCTGTCTTTTCTTCCCTCGCCGGCAGTAAAGGAAACCTTCTCG |
| | | | | | | 404 | |
| | | | | | 2189 | 38677 | CGTGCCCAAACCATTAGGAGATATCAATGTAGGCAAGCCTGAAGCTCACT |
| | | | | | | 095 | |
| | | | | | 2190 | 42620 | TTTTGCGTCTGGATTTCCTCCGTAGAGAGGCGCTGGGCCTGAGCCAAGCG |
| | | | | | | 091 | |
| | | | | | 2191 | 10061 | TGACCTTCCCTCTAAAAGGTAACACGTAGAGATTCCTAAAGGAACCATCG |
| | | | | | | 5670 | |
| | | | | | 2192 | 15955 | CGAGAGCACTTGAAAATGAAATGACTGTCTAAGAGATCTGGTCAAAGCAA |
| | | | | | | 8512 | |
| | | | | | 2193 | 14748 | CGGCCTCTGTCGGGAGAAATTCTGAGCAGAGCCGTTAGCTGCTCTAACGT |
| | | | | | | 230 | |
| | | | | | 2194 | 18831 | CGCAGAAGAGATTTACCCACAAGCAAGGTGGGAAAGAGTGAAAGCTTTGC |
| | | | | | | 2833 | |
| | | | | | 2195 | 53208 | ACAGCAAATGGAGCAGCAGAGTGCACACCACCAGACCCCGCTGGAGAACG |
| | | | | | | 752 | |
| | | | | | 2196 | 94083 | CTCAGCACCATAGCAGACAGGGTGGCAATACCATTACCAAAATAATACCG |
| | | | | | | 470 | |
| | | | | | 2197 | 14490 | CGGGCAGCTCGCCCGTCTTGAGCTCCCAGCAGAAATCGCAATCGCTTTAG |
| | | | | | | 3565 | |
| | | | | | 2198 | 22624 | GAATCTCAAGATCTGGGCAAAGGCTCGCGTCTCGGGACGCGAAGTCGACG |
| | | | | | | 094 | |
| | | | | | 2199 | 11953 | CGCCCGAGTCCTGCTTCCCACCCACCGGGGCAGGCGCTCACAGACTGTGT |
| | | | | | | 0677 | |
| | | | | | 2200 | 20359 | CGGGCTGTGCTCCTTTGACTTGACCTTGAATCTAGAGACTTGGTGGTAAA |
| | | | | | | 8954 | |
| | | | | | 2201 | 11711 | GGTGGAGAAAGCCGCTAGAGCAAATTTGGGGCCGGACCAGGCAGCACTCG |
| | | | | | | 9938 | |
| | | | | | 2202 | 65506 | TGTTAGAGATACTACTCACATCCCACTTCCACTTTATTACAGAGATTACG |
| | | | | | | 902 | |
| | | | | | 2203 | 46691 | AGGCCTGGCAGGCCGGGGCGCATTTCCTCCAGCTTCTGGAAGACTTGCCG |
| | | | | | | 543 | |
| | | | | | 2204 | 22094 | AGTGCACGAATGTCTCACAGGAGCAAGGCTGGCTCAAGTCTAATTTTCCG |
| | | | | | | 699 | |
| | | | | | 2205 | 19754 | CTGGGGCTGGGGGAACGTATTCCTTGCTTGCCCTTGGGGCAGGGTGGCCG |
| | | | | | | 448 | |
| | | | | | 2206 | 76540 | GGTGGCAGGAGGGCCGGCGGAGCGCCATGGCCTGCATCCTGAAGGTAACG |
| | | | | | | 574 | |
| | | | | | 2207 | 15110 | CGCCGGGAACTCATTTGGGGCGCAGCTCCACCGACCCCTCGGCTACTCGC |
| | | | | | | 7400 | |
| | | | | | 2208 | 53208 | CGGGGCAGCATGGGTACCCAGGGCTGAGAAAGACAGTCACGGTTTTCTTT |
| | | | | | | 709 | |
| | | | | | 2209 | 47730 | CGCCTGCCGGCAGGGATGGGGCTCCACGGGCACCACAGGGTTAACAGCCC |
| | | | | | | 821 | |
| | | | | | 2210 | 13865 | CGCCCACAGGCTGACCCTGCAACCTCTGGCCCGGTGGCTACCTTCTGCTT |
| | | | | | | 8704 | |
| | | | | | 2211 | 26600 | TCTATCACTTCATGCAACAGCCTGGAAAATGATCAGAATTTTGGTCTACG |
| | | | | | | 049 | |
| | | | | | 2212 | 12145 | TTGGTAACACCTGGGAGCTCGTTAGAAATGCAGAATCCAGGCTGGGCACG |
| | | | | | | 4837 | |
| | | | | | 2213 | 15039 | TGGGTGGGTTTAACTCAGCTAGCAGCTGCGGCGGTCTTGGCGCGCTCTCG |
| | | | | | | 6241 | |
| | | | | | 2214 | 17554 | TTCTTGCGGCTGCTGGCGGGGGGATCGGGTGGGGGTGTTCCCTCTGTTCG |
| | | | | | | 8020 | |
| | | | | | 2215 | 20184 | CGACGGACAGGAAACGGTGAAAACTACACCCCGGCAAATGCCCCAACAGG |
| | | | | | | 350 | |
| | | | | | 2216 | 17699 | CCAGGTCTTGAGGGGCGGGTCAGGGGCGTGACCAGAGCGGCCCGGAACCG |
| | | | | | | 740 | |
| | | | | | 2217 | 13484 | TCTGTCTCGGTGATGCTAGGGACTTTTCCTACTGAAATGACCTGAGCCCG |
| | | | | | | 7724 | |
| | | | | | 2218 | 50794 | GGGCTGTGGCCCTGAGTGTGGTGCATCTGGGTCACACAACAAAACCCTCG |
| | | | | | | 057 | |
| | | | | | 2219 | 85641 | CGGCGACCGGACCCCGAAGCGCACTACTTGCCTGCCAACGCCTTGCTCCG |
| | | | | | | 076 | |
| | | | | | 2220 | 26250 | CGCCCTGAATTATGTAGTGTGATAGGACTTGTGATCAGCTACTAGGTCTC |
| | | | | | | 936 | |
| | | | | | 2221 | 55444 | GCGGGGAGTGGGGCGTGGCAGGTGGCCGCGCCTGGGGCAGCGGGTGCACG |
| | | | | | | 061 | |
| | | | | | 2222 | 54345 | ATTTTTGTCCGAGCCCGGTTAAAAAACGGGTGCCTGAGTTGCGTGTGTCG |
| | | | | | | 862 | |
| | | | | | 2223 | 38067 | CGCTTGGCTGCCTCAGCTTACACTTCCCAGCAGGATCTGGCTGGGAGCCC |
| | | | | | | 742 | |
| | | | | | 2224 | 14249 | CGCTCTCCCAGCACCCAGAACCAGGAGTCCTCCGCCCAGACGCCCAGCAG |
| | | | | | | 4439 | |
| | | | | | 2225 | 13092 | TGGCCCCTGTCGAGTGCAGGGCCAAGGCTTGTGTTCAATCGTGTGAATCG |
| | | | | | | 9940 | |
| | | | | | 2226 | 89867 | CGGGCAACTTCCATTCTGTGGATCCCGCAGTAACCCGGACATAGTAGGTG |
| | | | | | | 704 | |
| | | | | | 2227 | 13910 | CGCGCTTCCCAAAACGAAGAGCAGAGCTGACACCTTCCACATCGTTCCCG |
| | | | | | | 569 | |
| | | | | | 2228 | 17696 | TTAAGGTCAAGTTAGTGTCTTATCTTGGGTGACTCAAAATCACCGCATCG |
| | | | | | | 9223 | |
| | | | | | 2229 | 23413 | CCAGCTCAGCATGTAGCAAGGACCACCGAGTGCCAGCCTGGTTCCCTACG |
| | | | | | | 37 | |
| | | | | | 2230 | 10912 | CTGGGCGCCAGGTGGGCTCCTGGGAGGGTGATGAAGCAGGTAGGGCCTCG |
| | | | | | | 331 | |
| | | | | | 2231 | 85424 | CGTACCTGGGAGTTAAGTGCGCTCCGAAGGGTGGCTTCTGAAAGGTCCAA |
| | | | | | | 469 | |
| | | | | | 2232 | 45792 | CTCCTGGCTGGGGTGGCGCTGCAGGGAGAACCGCGAGCTCTCAGGGGTCG |
| | | | | | | 660 | |
| | | | | | 2233 | 11138 | CGCCCTACCATGGCTGGGCGCAAGCGGCCCCCGAGGACACCCCCGGGCCC |
| | | | | | | 3576 | |
| | | | | | 2234 | 45404 | CGGGCATGGGGCGCCAATACGTGACAAACCGTCCGTATTGCGCCCTCCCC |
| | | | | | | 157 | |
| | | | | | 2235 | 92413 | CGCTCGGCCCCTCTGGAATAAAACACCCGCGAGCCCCGAGGGCCCAGAGG |
| | | | | | | 706 | |
| | | | | | 2236 | 17030 | CGTCGCCTCCAGCCCCGACCCCGGTAAGTGAACGGCCCCCTTCGCTGCCG |
| | | | | | | 3721 | |
| | | | | | 2237 | 13650 | AACCTTGGAGACCCTGGCCCGCACGCGGAGTGGCGTAGTGCTAGCTACCG |
| | | | | | | 9643 | |
| | | | | | 2238 | 68260 | CGCGAGTTCCACCAGTCGGGCTGCGTTTTGTGAACATGGAAACCAGATTT |
| | | | | | | 574 | |
| | | | | | 2239 | 15077 | CGGCCCTCCCTAGCCTGGCGCCTCAAGCTCGCCGCCCGGCTTCCCCGTGA |
| | | | | | | 562 | |
| | | | | | 2240 | 32015 | CGGCACCACCTGGAGCCGACCATCCTTATCCTTGTACTGGACCACGAAGG |
| | | | | | | 635 | |
| | | | | | 2241 | 45914 | ATGGAGCCGCCCTCTCCCCAGGACCACAGGGGCGCTTTCCAGTGAAGGCG |
| | | | | | | 525 | |
| | | | | | 2242 | 17513 | ACTTGGACAGAAAGATTCGAGGCAAATATTCTAGGCTGGGAAATTTCGCG |
| | | | | | | 8377 | |
| | | | | | 2243 | 32173 | CGGAAACACTTGGAAGCTGAACATCAGCCCGAGGACTCACTATGCTTCCC |
| | | | | | | 084 | |
| | | | | | 2244 | 12433 | AAAGCTTCCAACTGTTCCCTTCCCTGGGTGGCGGCAGCTGCCCAAGGTCG |
| | | | | | | 9995 | |
| | | | | | 2245 | 71702 | CGCCTGTGTGCCCACTCAATAGCAAGCCCAAAGCATGCATCCCAGAACAG |
| | | | | | | 722 | |
| | | | | | 2246 | 11207 | CGCACAGCCTGCCTAGCCCTAGCCCCGTACCCCTCGGGGGTATGCCCTCG |
| | | | | | | 3398 | |
| | | | | | 2247 | 27064 | TTCCCCCTCGAAGCGTTTCCACTCAGTTCTTGATCATTCATGTGAAAACG |
| | | | | | | 968 | |
| | | | | | 2248 | 49576 | TCTTACAGTGGCAGCTTGTTGCTCTTTGATGCCCTGAGAGGGCATTTCCG |
| | | | | | | 235 | |
| | | | | | 2249 | 56303 | AAGGAAGGCTTCGGTGACTCAGGTGAGCGCCTTTCTCTTCTTGGCAGCCG |
| | | | | | | 48 | |
| | | | | | 2250 | 12405 | ACATCAGTCTTTCCCCCGGGTCTGGATGGATCCATCTGTGATAGATCACG |
| | | | | | | 5251 | |
| | | | | | 2251 | 20185 | CGGCCAATCTTTCCAAAATGCGCTCTCACTCTGTTGGCTGCTCCGGCAGG |
| | | | | | | 382 | |
| | | | | | 2252 | 56534 | TTTTTGGTTTCTCTTCCTCATCTTCTTTGTTCTTTGATATTGCAGTCTCG |
| | | | | | | 05 | |
| | | | | | 2253 | 17068 | CGGAGTACTTGTAAGGTTACTGTTACTCCAAAATGTTTGTACCTACTATG |
| | | | | | | 5661 | |
| | | | | | 2254 | 22904 | GGGGCGGTGGTGCTGGGCCTGCTCCCCCAGCCGGGCAGGTGGCCTTTGCG |
| | | | | | | 5417 | |
| | | | | | 2255 | 15067 | CGGCCATGTCTTCCATCTCCAGAATCTTCCCAGCAATCAGGAACAGGATG |
| | | | | | | 457 | |
| | | | | | 2256 | 13952 | TAGTAAGGAGCCCACCCTTGGATCTTGGAGAGAGCTCCCTTTTAATATCG |
| | | | | | | 329 | |
| | | | | | 2257 | 75552 | CGGCTCTCACTAGCTCCGCAGCCCCGTCTATTTTTAGCTCGTGCCCACCC |
| | | | | | | 98 | |
| | | | | | 2258 | 26757 | TTTATGACAAGAAGACCTCTCGGCTTCTTGTAACTGGGGATTCAACCTCG |
| | | | | | | 644 | |
| | | | | | 2259 | 56484 | TGGCATAAACTGGCCACTTTCCTCCATCCTGGCATAAAATGGAGGCACCG |
| | | | | | | 117 | |
| | | | | | 2260 | 50791 | CCGAGTGGTAGTCCCTCCGGGGGTCAAGGCCCGAGAGCTGGGGCAAGGCG |
| | | | | | | 419 | |
| | | | | | 2261 | 10485 | CGCTGAGAAAGGCAACTCGGAGGCGTAGAAAATGGATTTCTGAGCCAACG |
| | | | | | | 2446 | |
| | | | | | 2262 | 97323 | CGGCCCCCTCCAGGAACCGCCTCCCCCGCCAGCCAAGACTCCATTGACCA |
| | | | | | | 948 | |
| | | | | | 2263 | 45451 | CGCCAGGACACCGCCTTTTGGACTGGGGCTGAATTCTGCCCCTTGAAGCT |
| | | | | | | 57 | |
| | | | | | 2264 | 39694 | CGGGCGCAGACCTGCCCTACCTGCCCTCCAACTGGGCCAACACCGCCTCC |
| | | | | | | 709 | |
| | | | | | 2265 | 22949 | CGAAGGCCAGTGGGAACCGTGAGGAGACGCACAACCTGGTTTGGGCCACG |
| | | | | | | 61 | |
| | | | | | 2266 | 10091 | GTTTTTCAACCCGAGTTTCCAATCCAGTGTTCATCTCCAGAGTTAGAACG |
| | | | | | | 7022 | |
| | | | | | 2267 | 13191 | CGTGCTAAAGATGGCACAGATGGGTTACATCCAGGCAAAACTTGGGCATT |
| | | | | | | 4037 | |
| | | | | | 2268 | 11112 | CGGGCCACCCCTGCCTCTGGGTCTCCTGATTCCTTGGCCTCGTCCCCTGC |
| | | | | | | 7346 | |
| | | | | | 2269 | 61822 | CGCCCAGGCCAGCTCAGGGTGTTCCCGTGGGGGTCTCCGGAAACAATGGT |
| | | | | | | 740 | |
| | | | | | 2270 | 18254 | CGCCATATGGTCTTCCCGGTCCAGCCAAGAGCCTGGAACCACGTGACCTG |
| | | | | | | 5492 | |
| | | | | | 2271 | 37123 | CAATATTCTAGAGGCCGCGGGGACGCCTGGTGGGTAGGGCTGCAGCTCCG |
| | | | | | | 638 | |
| | | | | | 2272 | 32223 | CGGCCTGGCGGTCCTACTCAACACCGCAAGATTTCAAAAGGGAAATTCCT |
| | | | | | | 090 | |
| | | | | | 2273 | 25058 | CGGCCATCACGCTGCTGCCGCCCCAGCGCTTCTCCCGCTTGCGCCATTTG |
| | | | | | | 429 | |
| | | | | | 2274 | 16169 | AAGGTCCACGTCGGGTTTCAGGGCTCCAGCCGATTGTTCCAAGGGCGTCG |
| | | | | | | 7241 | |
| | | | | | 2275 | 88877 | TTGGATCGGGAGAGGATCGCACGGTTTCCACCTGACGGAGCCACGTCACG |
| | | | | | | 301 | |
| | | | | | 2276 | 11067 | CCACGGGTCGCATCTTTCAGTGCCCAGATAGACTGAAGTGCGGCCAAGCG |
| | | | | | | 8956 | |
| | | | | | 2277 | 19789 | AGCTCAGCTGCGGTGGATACTCTCCTTGCAAGAGGGTCTCGAAGTGGGCG |
| | | | | | | 0608 | |
| | | | | | 2278 | 41712 | TAAAGACCCCCAAGCAGCACTGACAGTGAACTTGTGCTAAAGGTCTTTCG |
| | | | | | | 591 | |
| | | | | | 2279 | 13611 | CGCAGCCTGCCCTGAATTTTCTTCACACAGCCTGGGGGCATCCGAATGCA |
| | | | | | | 439 | |
| | | | | | 2280 | 68121 | CGCACTCCCGCCTTGGGAACCACAGGGTTCGTTGCTGGGAAGCTGTCTCG |
| | | | | | | 028 | |
| | | | | | 2281 | 13356 | GGGCCCCTTCCAGTGCTGCTGGGTGCAGGTATAGGCATCCGTCCCAGACG |
| | | | | | | 1756 | |
| | | | | | 2282 | 10044 | CGCGAGAGCAGAGATGCCTTCAGATTTTATTCGCGAAGGCACCCCACGCT |
| | | | | | | 2151 | |
| | | | | | 2283 | 11126 | ACTTCTGGAGTGTGTGTGTGCCAGGTGCGGGCGCGAGTGGCCGTGTGGCG |
| | | | | | | 79 | |
| | | | | | 2284 | 11513 | GGCGACATAGCTTCCAGTTAATTAGTCAAGAAATGAATCAAGTCCCAGCG |
| | | | | | | 6175 | |
| | | | | | 2285 | 13617 | CAAAGAGCAAAGGGCTCCGGGTTACGCTGCGGCGAACGATGCGGAAGACG |
| | | | | | | 091 | |
| | | | | | 2286 | 85472 | CGCCGCAGACGCTTATGGCTTAAGGAGGCCCAGGCTCTTGGGACCAGAGT |
| | | | | | | 949 | |
| | | | | | 2287 | 25962 | CGGGACAGAGAAGAATAGAAAGCTGACTCAGATGAATTTTGTGATGCCAA |
| | | | | | | 567 | |
| | | | | | 2288 | 55444 | CGGCATTTCTGTCGATTCCCGCAGGTGCCCCGTGCACCCGCTGCCCCAGG |
| | | | | | | 091 | |
| | | | | | 2289 | 12969 | TTATATCTGCGCCAGGCGGCGGCTCGAGTGAGGTCAGCGGGGAGGGGTCG |
| | | | | | | 3489 | |
| | | | | | 2290 | 85997 | TGGGGCTTTTTCCAAAACCAAGTCCCTTCCAGCCCTGCTTGTCCTCTTCG |
| | | | | | | 469 | |
| | | | | | 2291 | 47999 | CGCGGCGTGCATCACATTATTTCATTTCAAAGAATCCGCAGAGAAACCCG |
| | | | | | | 163 | |
| | | | | | 2292 | 89949 | CGGCCGCCCGCACATCTGGCCCCTAGACCTGCTCGGTAGAGGACTGGCTG |
| | | | | | | 776 | |
| | | | | | 2293 | 25506 | GGACCGTCGCCAGAAGGCGCCCAGAATTGGGTCCCTCCAGGTGGGGAGCG |
| | | | | | | 131 | |
| | | | | | 2294 | 32713 | GGAAACAACAGTCAGAGAAACCCCAAGAGCGGGCGAGCTAAGTGGCGAC |
| | | | | | | 112 | G |
| | | | | | 2295 | 42733 | CGGGCCAATCCCGAACACATTTACTTTGAGCGGGAAAAGCTCAGAAGCGG |
| | | | | | | 542 | |
| | | | | | 2296 | 42688 | CTGTGCCCGGGAGCTCTGAGGTCGCTGGCGGAGGGTGGACCCGTTCCCCG |
| | | | | | | 540 | |
| | | | | | 2297 | 86038 | CGCCGAGCCTCAGCTCACACACTCCGCAGACCCTCCTGCGCCCACACCCA |
| | | | | | | 802 | |
| | | | | | 2298 | 15358 | CGCGCAGTCAGAGCTGCCTCTCGCCCTCGCTAGCTGGGCTCGCAGCCTCT |
| | | | | | | 4416 | |
| | | | | | 2299 | 37571 | CGGCCCTGGGTGACAGACACAAGCTTCTGAACCCAAGCTAACAAAGGCGG |
| | | | | | | 677 | |
| | | | | | 2300 | 10274 | CGGGGTTGCTGGTCTCCTGGTCGCACCAAGAAGCATCGTTTGGGGTTCAG |
| | | | | | | 8988 | |
| | | | | | 2301 | 30037 | GGGGGGCCAACAGTTGTTATTTTCCCAAACCCGGGGATGCGGTGAGTGCG |
| | | | | | | 875 | |
| | | | | | 2302 | 11913 | CGCGGCCTGAGAAAGGTGGCACAGCAGCTTCCCGCCCACGCGCCTCTCTC |
| | | | | | | 3852 | |
| | | | | | 2303 | 75595 | GCGACCAAGAGTCTGTACGAGTGACTCGGTGTTTCTTTCCAGCCGGTTCG |
| | | | | | | 970 | |
| | | | | | 2304 | 50883 | CGCCCTGACTGAAGGGCCCGCAGCGGAAAACGCTGCTGCAGAGCAGCACT |
| | | | | | | 329 | |
| | | | | | 2305 | 60288 | CGCGGGGAAGCGGACGCGAAGGAAAACCTAAGTTCCTAACAACTATGGAA |
| | | | | | | 082 | |
| | | | | | 2306 | 14062 | CGCATGGTGGCGCTGCAGGCTAAGGCTTCAAAAAAAGGAAAAGGAAAAA |
| | | | | | | 4888 | G |
| | | | | | 2307 | 62305 | TATCTGTAATTTAGACCCCGGAGGGCGGGCCTGTGAGCGATTGCTGTCCG |
| | | | | | | 147 | |
| | | | | | 2308 | 27787 | TACAAATACAGAGGGCGGCCCAGCCACGAGGCCCAGCTGAGGGCACGGCG |
| | | | | | | 026 | |
| | | | | | 2309 | 24902 | GTGCCCAAGGGCCACAAGAGTATGACGGGGCTGTACGAGCTGCTGTGACG |
| | | | | | | 376 | |
| | | | | | 2310 | 10090 | CGGCCTCATTAGTATGCGCCTACGAGGGAAAATGAGACTTCCCCACAGGT |
| | | | | | | 3817 | |
| | | | | | 2311 | 11193 | GTAGGCGGGCGTCAGCGGCGCGGGCTTGCTGTCTGCGTGCTTGCTGGACG |
| | | | | | | 5508 | |
| | | | | | 2312 | 15640 | CGCGTGGCCCCCACCCACTCAGCTCCCAGCGCGGAGTCCCGGGACCACCT |
| | | | | | | 0281 | |
| | | | | | 2313 | 10280 | TCAAAGACTTTCAAGCCACTTCCTAGTGTGATGTCACATTCCTTCCTACG |
| | | | | | | 3407 | |
| | | | | | 2314 | 23822 | CGAGAGCACCTGTAGTTGGGATTTTTTTCATTGCAAAGGCATCGATTTTA |
| | | | | | | 666 | |
| | | | | | 2315 | 10257 | AGCCAGAATAGATGCCCGGAGACTCCGGAGTCCGCTGTTCAGGCGCTGCG |
| | | | | | | 2229 | |
| | | | | | 2316 | 72298 | CCAGAGGCTAAAAAGCTCAGCTGGAAAGCTACAGTGGGGCGGTGCCAGCG |
| | | | | | | 424 | |
| | | | | | 2317 | 13651 | CTGGGCACTTAGTGTCCAGTTTCCACCCAGAACAAGGTGGCATTCTGGCG |
| | | | | | | 0760 | |
| | | | | | 2318 | 38965 | TCCAGCAACACAAAGCGAAAAGTTAGTGGAGGACTACGAGCGCGATCTCG |
| | | | | | | 026 | |
| | | | | | 2319 | 11328 | CTTGCGAGAAGACCCCTCCCAGCCCAACCAGGGTTAGGGACGGGTGACCG |
| | | | | | | 6757 | |
| | | | | | 2320 | 99277 | AAGCTCTTGCTAGAGCTCCTGGCTTCTCTCATCCTCTGGAGCAATTTTCG |
| | | | | | | 015 | |
| | | | | | 2321 | 13356 | CGCGCAAAACTTCCATCCTGTCCACGTGAAGTTGTCGCTGCCTTAGAGAG |
| | | | | | | 2492 | |
| | | | | | 2322 | 13532 | AAGCATTTGAATGGACCTCAGCCCTAGGACCCTGGCGCCCTCGGAAACCG |
| | | | | | | 951 | |
| | | | | | 2323 | 29886 | CGCCCCGCCCGACCTCGGGTCGCGGGCTCCAAACCCAGCCCTCCAAGGGT |
| | | | | | | 890 | |
| | | | | | 2324 | 81107 | TAATGGAGGGTCGGGCCAATAAATTAGCTTTAGGAACAGACGGACGGACG |
| | | | | | | 087 | |
| | | | | | 2325 | 22959 | CTCAGGCATGCCTGGCCCAAAGCAGGGGTGCAGGCAGCTTAGGGATGACG |
| | | | | | | 875 | |
| | | | | | 2326 | 15319 | TTGTGTGATGGCTCATGACCTCCGGGCTGAGTGGCTTGGTCTCCCCCTCG |
| | | | | | | 9588 | |
| | | | | | 2327 | 18254 | CGCCCTGCAGCTAGCGCTTTTGTACAAGGTGGAAATTTAGCGGTGGGCCA |
| | | | | | | 4321 | |
| | | | | | 2328 | 13702 | CGGGGAACCTGATCCTGCCTTTGACTCAATTACGCCCTGACAGGGAGGGA |
| | | | | | | 8410 | |
| | | | | | 2329 | 42543 | GTCACCCGGGAGCAGGGAACACCAAGGAACCCAGAGTCGGGCCTCTTTCG |
| | | | | | | 530 | |
| | | | | | 2330 | 14937 | CTCCCTGCTTCCAGCTCTCCATATCTGTGAAATAACCGTGGAAGGACACG |
| | | | | | | 4535 | |
| | | | | | 2331 | 14030 | CCGGGCTTGCTAGGCTGGAGGCTGGGCAGCCGTCTGGGAAAGCGGAGCCG |
| | | | | | | 6231 | |
| | | | | | 2332 | 13382 | CGGCACTGGGAGGGCTGGTCATTTGTTTTTCAAGCTGTGGAGATTCCACA |
| | | | | | | 4178 | |
| | | | | | 2333 | 97646 | GCTGGTTTTTTGGAACCTCTGCAAACACTTTCTCCTGGGTCAGTTTCCCG |
| | | | | | | 811 | |
| | | | | | 2334 | 11334 | GAGGCTGGCGGTCACTGGTCAATCACAGGTTCGAGAAGTCGCATGCACCG |
| | | | | | | 5944 | |
| | | | | | 2335 | 62660 | CGCCGGGCCCCATGCACCCGGGCCAGCCTTACCTGGCGAAAAAGGAATCC |
| | | | | | | 624 | |
| | | | | | 2336 | 13160 | TCCACAGGAACCGCGGAGGACAGAGCATGCACATAGGTATGCCAACGGCG |
| | | | | | | 9432 | |
| | | | | | 2337 | 16216 | GAGTTGTGCAAAGCCTGGGAACAGTGAGGGCATTAGGACCAGGAGTGACG |
| | | | | | | 094 | |
| | | | | | 2338 | 84814 | CGCGCTCATCAGAAGTTTAAACAACAGCCACCAACCCGCGAGGGCAGGGC |
| | | | | | | 60 | |
| | | | | | 2339 | 59000 | CTACCCCCTCTGTCAGAAGTGTCACAGCTTTTCTGCCTTCGGATGTTTCG |
| | | | | | | 860 | |
| | | | | | 2340 | 13617 | CGGTGCTGCTTGTTCATGAAGACTTGTGCTGCAGAATCCAGCAACTTCAT |
| | | | | | | 3085 | |
| | | | | | 2341 | 14613 | GGTACACTGCAAAAGCGGCTTCAAGTCCCCTCCCCAGCTAATTACCTTCG |
| | | | | | | 6563 | |
| | | | | | 2342 | 50883 | CGCTCTGTTCCTGCTTCAGTGCGCACCTTGACGGCTCCTTAGGGACAGAC |
| | | | | | | 394 | |
| | | | | | 2343 | 23313 | CTGTACTGGTGAGCGCGATGCTTCGCCTAGGTGAACGGGACAGGTACACG |
| | | | | | | 293 | |
| | | | | | 2344 | 65360 | CGGCGCCACTCACCAGACTTGCCAGCCCCGCGGCGCCCCAGGATGGCCAG |
| | | | | | | 058 | |
| | | | | | 2345 | 20806 | CCGTGGCGGGGGCGCAGGGTGGGGGACCCCTAAGAAGTGCACAGTGCGCG |
| | | | | | | 732 | |
| | | | | | 2346 | 44063 | CGCAGCCCGGTGACCACCCCGAGGAGCGCGGCTCCGTTACTTACCCTGTC |
| | | | | | | 593 | |
| | | | | | 2347 | 12113 | GACTTTCTTTTCACGCGCGCGCCCAGAATGGACTGGGGACCTTTGGGACG |
| | | | | | | 7734 | |
| | | | | | 2348 | 16228 | CGCTTCTCCACGAGGTAAAGAGTTCTGCGGACTTCACTAGACGCAGCGCC |
| | | | | | | 3189 | |
| | | | | | 2349 | 14224 | GCTGGAGGTGAAACGGGCAGCCTGAGGCCTCCAGCTCACTTGGAGATACG |
| | | | | | | 7880 | |
| | | | | | 2350 | 24803 | CGGGGCTGGGGAGATCATGGCCCGCCTCTTCAGCCCCCGGCCGCCCCCCA |
| | | | | | | 873 | |
| | | | | | 2351 | 11900 | CGCAGTAAATTCCTAAAGGCTTGCAAGAGAAAGTGGGGAATAAAAAAGTC |
| | | | | | | 6837 | |
| | | | | | 2352 | 14712 | CGCGCAGTTGTTGAATAGACTTTGCGCTAGGCAAAGACAGGTTAATCGAG |
| | | | | | | 5712 | |
| | | | | | 2353 | 29235 | CTGGGCACCTCTCATTTCGGAAGGCCATCAGAGGCGCCCACTACTGAGCG |
| | | | | | | 123 | |
| | | | | | 2354 | 15262 | CGGGCAGCCCCACAGTGGGCGATTTTCCCGCACTCCGCACGAGTCCATCC |
| | | | | | | 3043 | |
| | | | | | 2355 | 15162 | CGTGAAATTGTCATTCTAGGGGTGATATATTACTACTTCATGGATTATCA |
| | | | | | | 821 | |
| | | | | | 2356 | 42074 | CGGAGCTGCGCTCCTCTGGAACAAAGCAGCCAGACGTGGCTTGAGTGGAT |
| | | | | | | 635 | |
| | | | | | 2357 | 22509 | GGGCGGCGGGAAGGGCACGGTGTCCAGCTGGCCGTGCGCAGGCAGCGGCG |
| | | | | | | 56 | |
| | | | | | 2358 | 10887 | TGCGGGGAGACCAGGCAGAATAGAAACCTCGGATCCCTAGCTAAATAGCG |
| | | | | | | 587 | |
| | | | | | 2359 | 22865 | CTAACTCCGTCAGAGCGCAGGCTTATAGAAGGCACTGTCTCCGAGCTGCG |
| | | | | | | 3136 | |
| | | | | | 2360 | 14081 | CGCAGCTGCTTGTAGTCAACGCTACCGACCCTGACGAAGGAGTCAATGCG |
| | | | | | | 1102 | |
| | | | | | 2361 | 91580 | CGCCCCAAACAAACAGTAAGTCAGAAGGGAACAGAGCACAGCACAGTCCC |
| | | | | | | 613 | |
| | | | | | 2362 | 10886 | GCAGCTCGAGCAACAGGCGGCTCAGAAGGGTTCGCAGCCGCTGGCTCTCG |
| | | | | | | 8237 | |
| | | | | | 2363 | 13164 | CGGGGACTTCTGGTCTCCTCTGTCAGGGGACAGGCAAATCGAGTGACTAG |
| | | | | | | 0135 | |
| | | | | | 2364 | 35991 | ACAATATCTACACTGGGTGAGTGCTTGGCCGGAGAATTCCCAGACAGGCG |
| | | | | | | 215 | |
| | | | | | 2365 | 66123 | CTGGGCGCGCAGGAGGTATAGGACTCTGGGATTTTTTGGCCAAAACTCCG |
| | | | | | | 127 | |
| | | | | | 2366 | 26687 | CGAACAAGGGAGTCTTGATTAGTACATTTGGGATTCTGGAGATGGCAGAA |
| | | | | | | 962 | |
| | | | | | 2367 | 16906 | CGGCTTTCCCGCCCCGAGGCCCCGCCGCAGGCTGCGCCCTCTGACCTGCA |
| | | | | | | 4530 | |
| | | | | | 2368 | 56415 | CGCAGACATGCATACACCACCCGCACTCCCTCGCCGTTTCCAAGGCGGCG |
| | | | | | | 271 | |
| | | | | | 2369 | 11513 | CGCCTAGGCGTCCAAGCTGTGTTCCCCACCGCGCCCCACTCCGCCCAAGG |
| | | | | | | 6287 | |
| | | | | | 2370 | 11708 | CGGGGCCCCCAGCAATTCTGGGCCCCAAGGTCACGGTGAGCCAGGGCTCC |
| | | | | | | 6601 | |
| | | | | | 2371 | 11421 | CTTCCAAAACAAGGCACAGCTTAGGAAAATCACTAGTTTTTACCATTACG |
| | | | | | | 4449 | |
| | | | | | 2372 | 66664 | CGGGTACTGATGCGAGTGCAGAGGAGGCCCGTGGTTCAGGTGGTGGACCG |
| | | | | | | 960 | |
| | | | | | 2373 | 10100 | CGCCACTGCCGCCTCCTCGGCCCCGGACCGCCGAGGGTTAACAAAAAAGC |
| | | | | | | 6035 | |
| | | | | | 2374 | 78173 | CGGAACTATCAACTGGGGACAAACCTGCGATCGCCACGGTCCTTCCGCCC |
| | | | | | | 227 | |
| | | | | | 2375 | 26674 | CCCTGAAGCCGCATTGGTGGGTCTCCATTTAGTTTGTCCTTTAAAGTCCG |
| | | | | | | 085 | |
| | | | | | 2376 | 72740 | CGCATCTGAAATTTAGATCTGAAAATAATTACGAAGAGAGCCAAGAACAA |
| | | | | | | 693 | |
| | | | | | 2377 | 14712 | GTCACCCTTGTCACAGTTATTGAAATTCCGCAACTCGCCACACCAGGACG |
| | | | | | | 5942 | |
| | | | | | 2378 | 12669 | CGCTTTGGTGGAACAAGTGATTACAGGAACAGTTCCGGCAATTTAGAGAC |
| | | | | | | 584 | |
| | | | | | 2379 | 25041 | CGCCCCTTGTCAAATCGCCTCCAGGGTCTCGGGGACCCGGTGAACAAAGC |
| | | | | | | 134 | |
| | | | | | 2380 | 18841 | CGCAATCTGATCTTACTAGCAGTGAAAGAGCGAGGTAAGAATTTGACTAT |
| | | | | | | 9087 | |
| | | | | | 2381 | 31938 | GACCCCTCTGGGGAGGTTAACACCAACGTGGCCTTCTGCTCTGTGCTACG |
| | | | | | | 533 | |
| | | | | | 2382 | 72530 | ACAATACTTTTTAACCCGATGGTTACCCCTCTGACTCCGATGACCTTTCG |
| | | | | | | 472 | |
| | | | | | 2383 | 11217 | CGCAGCTTTCAGACCTGCAGTCCATGTTAAAAGGATCTTGTTTCCAATTT |
| | | | | | | 38 | |
| | | | | | 2384 | 48565 | TTGGGGCCGGTCCTGGGTGGGAGGTGGGCTGCGGCGAAGCTGACTTTGCG |
| | | | | | | 189 | |
| | | | | | 2385 | 11803 | CGCTGCAGCCGAGGGGCGCGCCTGGCTGATGTGTGGTTGAATGGAGAGCG |
| | | | | | | 4031 | |
| | | | | | 2386 | 19184 | CGCCTGGCCATCGTCTATATCTCCTTCATGACCGAGCTCTTGGAGAGCTG |
| | | | | | | 555 | |
| | | | | | 2387 | 51841 | CGCCGTTCCTCCCACACGTGTCTGAGGTGTCAGTCGCCATGGGAACAGCC |
| | | | | | | 55 | |
| | | | | | 2388 | 10335 | CGGCTGAGCAGAGCTCCAATCTAGAAGTGTCCGGGGTTTTGTTAATTGTA |
| | | | | | | 9836 | |
| | | | | | 2389 | 44313 | CGCTTCTCACAGACCTTGATTCCTGAAATGATGCAGGAGCAGGGTCAATG |
| | | | | | | 578 | |
| | | | | | 2390 | 79470 | GGTCCTTTCGGGGACTGCGGCTGCTTCCCTGTTCGTAGACTCAGCAGCCG |
| | | | | | | 966 | |
| | | | | | 2391 | 81471 | CGGCGACTCTAGTGTGGCTTCCAAACTTCTGGGCTCTGGGCTCACTTCGG |
| | | | | | | 754 | |
| | | | | | 2392 | 70035 | TTCCCCATCAAGTCATAAAGCGAGCGGGTTGGGCGAGAGGGCACAGCTCG |
| | | | | | | 479 | |
| | | | | | 2393 | 23453 | CGCGGGTGCACCCGTGTCTCCTGGACACAGAGGCTGCCCTCTGATTGGGT |
| | | | | | | 98 | |
| | | | | | 2394 | 23453 | CGCCCCCGCGCCCCTTTGGAGTTCTGACCCCGCGGGTGCACCCGTGTCTC |
| | | | | | | 68 | |
| | | | | | 2395 | 16780 | CCCCAGAAGCCTGTTCCTGAGCTGATTGGGGTTCTGGGCCAGGGGTCTCG |
| | | | | | | 9876 | |
| | | | | | 2396 | 17330 | CGTTGTTTGATCATTTGTATTTCTTGCCTGATGGTCGGATTTACATGGAG |
| | | | | | | 2427 | |
| | | | | | 2397 | 13387 | CTCCTTCCTGTGCAGGTATCTCTGCTCTTTCGAGTACAGCAGGGGTGTCG |
| | | | | | | 1212 | |
| | | | | | 2398 | 22094 | CGCCCGCCCCAAGTCAAAGCTGAGCAGAGCAGGGATGGGGAAGGTCTCCG |
| | | | | | | 371 | |
| | | | | | 2399 | 11062 | TTTATTAAATGGTCCACACCTGAAAGTAATTGAGTCTCGGCGTGGGTGCG |
| | | | | | | 6540 | |
| | | | | | 2400 | 40618 | CAATGTATTCCTAAAAGGGGCCATCTGGGCGGTGTCCTCTTTTCCCAGCG |
| | | | | | | 284 | |
| | | | | | 2401 | 13974 | TCGGCCAAGGCCGAGCAGCACGGGAAGGTCGGGGAATCCTGGGAAGCTCG |
| | | | | | | 614 | |
| | | | | | 2402 | 10089 | ACACTAAAGATGAACAAAAACCAAGGCTGGCGCTCACTTGCGTTGACTCG |
| | | | | | | 5679 | |
| | | | | | 2403 | 13141 | CGAACTACAGAAGTATGAATTGACTCAAGGGGGCTGGCAGTGATTTATGT |
| | | | | | | 0868 | |
| | | | | | 2404 | 26223 | GTGTCGGGACAGCGCAGGGGACTGGCTTGGGGCGGCGGTGGGTAGCGGCG |
| | | | | | | 310 | |
| | | | | | 2405 | 38821 | TTTCCCATCCTCCACCTTGGCTAACTGAACCTTATCGATAGGATGAGTCG |
| | | | | | | 403 | |
| | | | | | 2406 | 47225 | CGCCACTCATGGACCAAGTCTCCGCCGATCCGTAACAGCGCTGTGTTCAG |
| | | | | | | 852 | |
| | | | | | 2407 | 61616 | AGAGATGTCTGAGCGGCGCCGGGTGTTAGTTCCCAGTGAATGGGGAGGCG |
| | | | | | | 526 | |
| | | | | | 2408 | 25056 | CGCGAGCATCCTTGTTTATTAAACAACCTCTAGGTGAATGGCCGGGAAGC |
| | | | | | | 243 | |
| | | | | | 2409 | 22873 | TTTTCTTGCTCTCTTATCCCCGGCCGATGGGCTTCGTAGCCTTGGAAACG |
| | | | | | | 6357 | |
| | | | | | 2410 | 10256 | CGGTCTTAACGTGGTTACCGGTGCACTGAAACTCTCAGCAGCGGTTGGAG |
| | | | | | | 9876 | |
| | | | | | 2411 | 99226 | CGGTACCCAAATGTCAGCTGACTGTGAATAGGAATATGAAACTGGAAATT |
| | | | | | | 761 | |
| | | | | | 2412 | 10485 | CGGGCCACGTTGGCTCAGAAATCCATTTTCTACGCCTCCGAGTTGCCTTT |
| | | | | | | 2439 | |
| | | | | | 2413 | 17299 | CGCCTATGAATGTCAAAGTTCTGCTAGGTTTCTCTGAAGCCTGCTTCTGC |
| | | | | | | 350 | |
| | | | | | 2414 | 11945 | CTAATCAAAGACACGTCAACCATCCCCCGGGTTGAAATAATGGGTGTTCG |
| | | | | | | 7146 | |
| | | | | | 2415 | 20212 | CTAAATGGAGTCAGTATAAATGCTTTCCAATAAAGCATGTCCAGCGCTCG |
| | | | | | | 2653 | |
| | | | | | 2416 | 21490 | CAGCAACAAGGGTGGGACGGAATCCGTTCCCCGCCGGAAAGATGGATGCG |
| | | | | | | 665 | |
| | | | | | 2417 | 11529 | GTTGCAGAGCTAAGAAAAACCATCCCGCCAGGCCCCTTCTTGTGGTAACG |
| | | | | | | 7488 | |
| | | | | | 2418 | 27433 | TTCTGAGAACAGATCTGTAGTTTTCTAAACACCCTACATTGATCCAAACG |
| | | | | | | 191 | |
| | | | | | 2419 | 75601 | GGCGAAAGCGGAGCTGTTCTGTTTAAACGCCCTTTGTGTGTGAGGACGCG |
| | | | | | | 120 | |
| | | | | | 2420 | 37123 | CGCCCTTGTTAGTTATTCATGAGACGGAGGGCACCATGGGAGCTGGCTCT |
| | | | | | | 909 | |
| | | | | | 2421 | 20134 | GGGGCACGGCGCAGAGCTGAGATAAACGCCCTTTCCCTGGCCCAGGATCG |
| | | | | | | 719 | |
| | | | | | 2422 | 83678 | CGCCCAAGAGTCCACAATTTTCTTGTGACTCAAATAGATGGATAGGTGCA |
| | | | | | | 796 | |
| | | | | | 2423 | 44333 | TACGTTCTCGCTTAACGATTTTCTCAGCGGTTTAAGGAAGGCTGCTCTCG |
| | | | | | | 192 | |
| | | | | | 2424 | 70682 | CGCGGGCATCGAGGATTCTCAGCTTTTGGCGTCCCAGTCCTGCCTCTGCT |
| | | | | | | 127 | |
| | | | | | 2425 | 18143 | CTGCTCTAATCCAGAGGGGCCCAATATTCCAGGGATCCCAGATTGCTCCG |
| | | | | | | 127 | |
| | | | | | 2426 | 11138 | CGCGGTTTCTCCAGATACAGTTAAACTGTTAGCTCTCTCTAGGAGTCACA |
| | | | | | | 3889 | |
| | | | | | 2427 | 88717 | CGGGGAGTTCCTGCGCTTCCCCAGGGCTTCCCAAACTTCAGCTCTCAAAC |
| | | | | | | 646 | |
| | | | | | 2428 | 46526 | GGAACTTGTGTATTTTACAACTGCGATGCCTATCGGGAATGTGGCAATCG |
| | | | | | | 785 | |
| | | | | | 2429 | 49087 | GTGAAAATCACCAGGAATACGTACTCCACCTGCTCCTGGGGGTGGGACCG |
| | | | | | | 465 | |
| | | | | | 2430 | 14748 | CGCCAAGCTCTTGAACACTCCGGCCTCTGTCGGGAGAAATTCTGAGCAGA |
| | | | | | | 250 | |
| | | | | | 2431 | 13310 | CGGCCACTTCCGTCGGTTGGAATTCTCTTAAGCATTGCCCTGAGTCTATT |
| | | | | | | 9029 | |
| | | | | | 2432 | 47572 | AAAGCCCATCTAGGGAATCTCTCATTTAGGATCCATGGTCATATGAATCG |
| | | | | | | 921 | |
| | | | | | 2433 | 22956 | CGAGACCACCTACAACAGCATCATGAAGTGTGACATCGACATCAGGAAGG |
| | | | | | | 7626 | |
| | | | | | 2434 | 11638 | CGGGGCGAGAAGTGCACCCCGCGTCTCCCCGCCCCCGTGAAAACCCAGAT |
| | | | | | | 1475 | |
| | | | | | 2435 | 13904 | CGCCTCCCAGACCTCTGGGGCCATGAATCATTTATGAGGCAAAAATGAAA |
| | | | | | | 7739 | |
| | | | | | 2436 | 15427 | CGATTCATTGGCCTGACCCTTTGAGCCCAGAGCAATGAAGTCTCCTGGGG |
| | | | | | | 69 | |
| | | | | | 2437 | 11051 | ACTTTCCACATTTTCCTCATGACTCAGGATCAGTTTCTGACTGCTGGGCG |
| | | | | | | 3817 | |
| | | | | | 2438 | 12197 | CTATAGAAGTGTAAAGACGAATAGCGTTCTGAACTCACCCATTCGAAGCG |
| | | | | | | 1092 | |
| | | | | | 2439 | 67218 | CGGAGGTCTGGGAGGCTCCGGGCGAAGCCTCCCTGCTGCAGGTAGGGTCG |
| | | | | | | 275 | |
| | | | | | 2440 | 10090 | GGGGCCTACGGAGGCCTTAAACCCCAAAGATTGGCGGGGACGAAGGGCCG |
| | | | | | | 3575 | |
| | | | | | 2441 | 79170 | CGTGGATTCGACTGTGCCGGGGAGAACCCGAGGTCAACGGCCTTAGGCCG |
| | | | | | | 283 | |
| | | | | | 2442 | 52156 | TAGAGGCTCCCCTTCCCCAGTGTGGGACTTAGCAGAGGGCGGGTCCAGCG |
| | | | | | | 170 | |
| | | | | | 2443 | 27772 | CGGCAACCATTTGTTCCTGGTGAGCCCCACCAAGGCCGGGCGCCTGGCTC |
| | | | | | | 44 | |
| | | | | | 2444 | 72756 | TGAAGCCCGACGGCCCTTCAACATGTCTGCGCAGGATAAGTGAGCTTTCG |
| | | | | | | 901 | |
| | | | | | 2445 | 13974 | GGGTCGGGGCGTCCCTGCGCACATTTAAAGGGGCCGTGGCCATGGGCACG |
| | | | | | | 286 | |
| | | | | | 2446 | 35479 | ATCCCTAGGGTGAGGAGCCCGAGGGGGTGCGCCCAGGCTTGGGGGTAGCG |
| | | | | | | 648 | |
| | | | | | 2447 | 17074 | CGCTTTCCTGCAGAATGTCATGTCACTGGCAAATGCCATGGAACCGCTCC |
| | | | | | | 6073 | |
| | | | | | 2448 | 64937 | CGGACATGATAAACAATCTGGAGAAATGGGAACCTTATTCACTTTGCTCC |
| | | | | | | 451 | |
| | | | | | 2449 | 10663 | CGGGTTTAGCTGAAGGCATTATGACTCCATGGCAACAGATGATTCCCTGA |
| | | | | | | 1934 | |
| | | | | | 2450 | 15371 | TTGGAGAAGCCCTTCTCCATTGTCTCAGTCACTTCAGGGTGCAGGAAGCG |
| | | | | | | 3930 | |
| | | | | | 2451 | 72520 | CGGGCTCTGCCAGTCTACACTTAAGCTCTTAAGGCAGTACAGAGAACCAG |
| | | | | | | 632 | |
| | | | | | 2452 | 73156 | CGTAGAGTTGCCACAGCAGCCGGTGCCATGACATCGGCAGAGGGCAGCTT |
| | | | | | | 377 | |
| | | | | | 2453 | 17991 | CAGGCACTTAGTGTCACCATACTAGGGGCGTGGCAATTCTCTCTCGTTCG |
| | | | | | | 4864 | |
| | | | | | 2454 | 91775 | TGTTTGTAGCTCAGGCACCCGGATGATTCATCCCATGAAAGCCTTGAGCG |
| | | | | | | 501 | |
| | | | | | 2455 | 10802 | CGAGCTCCTGCACTGCCAGAACGGAGGGACGTGCCACAACAACGTGCGCT |
| | | | | | | 3249 | |
| | | | | | 2456 | 18329 | CGGATATAGAAACTTCCACAACTGGGGGCCCTGGCTCTGACGACTTTCTT |
| | | | | | | 602 | |
| | | | | | 2457 | 10360 | GCCGGGCTGGGAAGACCTGAGGGATTAGACAGGGGTCCGGGCGGGGCTCG |
| | | | | | | 0846 | |
| | | | | | 2458 | 12426 | GGTCTACATTGTCACCATGGTAGGCAACCTTGGCTTGATCATTCTTTTCG |
| | | | | | | 7100 | |
| | | | | | 2459 | 40739 | ACATGGTTTGCATAGCTGGATCTCAGCTTATCTGTGCCTTTCGGCTTTCG |
| | | | | | | 661 | |
| | | | | | 2460 | 13779 | CGGGAGGGGGCTGCAAGGAGGCTGTCGCGCTGTTCCGGAGGGTGCCTGCG |
| | | | | | | 3853 | |
| | | | | | 2461 | 53919 | CAAGAATTTACATCCCACACTGAGTCATGCAGTAAGTTTAGTGCTTCCCG |
| | | | | | | 041 | |
| | | | | | 2462 | 54466 | CGCACCAGAGGCCCCGCTCTTCGTTCCAGCGCTTCAGCGACTCCAGTTTG |
| | | | | | | 441 | |
| | | | | | 2463 | 76834 | GGGCTGAGTTTGGTTGGGCAGGTGCTACGCGGAGCTCCTGAATGTAAGCG |
| | | | | | | 778 | |
| | | | | | 2464 | 23453 | GTCCAGGAGACACGGGTGCACCCGCGGGGTCAGAACTCCAAAGGGGCGCG |
| | | | | | | 74 | |
| | | | | | 2465 | 29186 | GTCCTCGTGACTGGGTGGGGCTGCCTCACTTCTGCCTGATTTGGGAAGCG |
| | | | | | | 110 | |
| | | | | | 2466 | 15814 | TCGCGAGCTCCATGTTCTCCTCTTTGGGACAAGTTGTTGAAATGGTTCCG |
| | | | | | | 3538 | |
| | | | | | 2467 | 10848 | TTACAGCCGGGCGCACAGAACACAGGATGCATATGCTGCGGGTGTGTGCG |
| | | | | | | 5102 | |
| | | | | | 2468 | 17513 | TCAAGGCCATGATTGCATCGCTCTCTGGAACGAAGATGGCCAGGAATTCG |
| | | | | | | 9486 | |
| | | | | | 2469 | 31070 | AGGAATAATGCTAATTAGCGAGGCTTAATGAGAACTTCCCCCAGGAAGCG |
| | | | | | | 190 | |
| | | | | | 2470 | 85667 | TGGAGGGAGCCATGAGCGACTGCCGTGCGGGACGTCTGGAATGAGGACCG |
| | | | | | | 353 | |
| | | | | | 2471 | 10304 | GGCCCCGGGCTTCCAGAGAAGTGTGTCATTATTTGTTTTCTGCAAAGCCG |
| | | | | | | 7225 | |
| | | | | | 2472 | 12457 | CGCACACCGTGCCCCACACGCCCTGGTGCCGCAGCTCTACCCGGCCCTCA |
| | | | | | | 8209 | |
| | | | | | 2473 | 80549 | ACGTAGACTTAATCCCGAGAGGCAATTGTTCCCTTGCTTGGGCGGCTACG |
| | | | | | | 634 | |
| | | | | | 2474 | 51522 | ACGGGAAGATGCCGCGAGGGGCGTCATTAGGGTAATTGTGCCCATTACCG |
| | | | | | | 561 | |
| | | | | | 2475 | 10053 | AGTGAGGTGTGTGTGACATATTCATGTGACCACCCGTGCAGCGTCACGCG |
| | | | | | | 98 | |
| | | | | | 2476 | 10009 | CGCCCGCGGCACAGCCCCACCCCGCGCTGCCGCTGCCTCTGCCCCTGCCG |
| | | | | | | 1255 | |
| | | | | | 2477 | 50602 | AATTGAGCTGCCCAGAATCCAGGGCGGAAGAGGCCCCTGCAGTGTTCTCG |
| | | | | | | 990 | |
| | | | | | 2478 | 20145 | TCACTGGCGCCCTCTATGTGGGCGGAAATCATCCGGCGAAGATGAAGACG |
| | | | | | | 0560 | |
| | | | | | 2479 | 17330 | TTACAAGACAACATGTGTGGCCAGCCACCCACTTCTACTGTCTCTATCCG |
| | | | | | | 3347 | |
| | | | | | 2480 | 11184 | CGGGGCTCCCCAGCACTCAGACCACTAGAGCCCTCCTCGTGATTCTGCCG |
| | | | | | | 7747 | |
| | | | | | 2481 | 39703 | CGGGAATCCCAAGTGAAGTTTAAGCCAATTGTTCAGGTTGTAGAGATGTC |
| | | | | | | 302 | |
| | | | | | 2482 | 11478 | ACATGACGGTCGGGCAGCTCACCCCAGACTCAGTGATGGCGTGGAAGACG |
| | | | | | | 0781 | |
| | | | | | 2483 | 13865 | CGAGGCGCTCCAGGCCGGGGTCTTCCCGGCTGCTGGCCCCTCTCGCTCCC |
| | | | | | | 8554 | |
| | | | | | 2484 | 13475 | CGCCGAGTGCAAAAGAGGCTGTTACTCGGAATTCGGCATGAATGTGGCCT |
| | | | | | | 4286 | |
| | | | | | 2485 | 27512 | TAAGATATTTGGAAACCTGTGTTCTGAGTTCTTCCTTTTCCCCGGGCTCG |
| | | | | | | 849 | |
| | | | | | 2486 | 13910 | CGCGCAGCTGCGTCTTTGGGACCTGGCACCGACTAAGCCGCTGCCTCTCC |
| | | | | | | 796 | |
| | | | | | 2487 | 46659 | CGAACAGCAACACGCATAATAGTAAAATAAGAGCAGGGGCTGCGCGCGAG |
| | | | | | | 318 | |
| | | | | | 2488 | 10249 | CGGGTTTGCGAAGGATGCGGGTTTTACTTAGTCTCGCTGAGCCTGTTCCG |
| | | | | | | 7666 | |
| | | | | | 2489 | 14712 | CGCTTGTTTTCGCGATGCGGCCCTCGATTAACCTGTCTTTGCCTAGCGCA |
| | | | | | | 5782 | |
| | | | | | 2490 | 12384 | GACTAACATATCATTTTTCCCTCTCTCCCCAGGGAGTACATGTTGTGCCG |
| | | | | | | 2309 | |
| | | | | | 2491 | 41746 | CGGCCCTGAAAGATGCCCTATCCATGAGATGCCTTTTCATCTGCAAACTC |
| | | | | | | 947 | |
| | | | | | 2492 | 72368 | CCTGGGCCTGGGGGCTTCAAGGAGATGCTCAGATTGACCCTTCCTGGTCG |
| | | | | | | 071 | |
| | | | | | 2493 | 31845 | TGACATATTGGGGCCTCGACAGCAAGGTTGGCCTCTGTCCCACAAGGACG |
| | | | | | | 389 | |
| | | | | | 2494 | 19295 | CGGTCAAAGCGCCGCGCAACCAGTTATTCCGCCCCCGGCTCCGCACCCGC |
| | | | | | | 8832 | |
| | | | | | 2495 | 38172 | TGAGCTCTCCAAAGCCCACTGCCAGTTCTCTTCGGGGACTAACTGCAACG |
| | | | | | | 876 | |
| | | | | | 2496 | 41882 | CGCCCTGCCGCGGAAACCCTTCCCGTGGCCTCGAACTGGTCCCCGACGAC |
| | | | | | | 163 | |
| | | | | | 2497 | 29974 | CAGTTCGTGCGGGTCGACAGTGACGCCGTGAGTCTGAGGATGAAGACGCG |
| | | | | | | 717 | |
| | | | | | 2498 | 29234 | GTATACTGAAGCCAGCTCTTGCCAACCCTGAGTGCTGCTGGAAAGCACCG |
| | | | | | | 671 | |
| | | | | | 2499 | 17759 | CGGCCCCGGCCCCACACCAGCCCTGGCCTCTTGTCTGGTTTAAATGATCG |
| | | | | | | 91 | |
| | | | | | 2500 | 12230 | CGCCGCTTCCCCGCACCCTCAGGGCGAGGGAAGACTTCCCTGGCTCACCA |
| | | | | | | 2265 | |
| | | | | | 2501 | 12331 | CGGGGTCGCTAAGCAATGCGGCACTCCCTGGCATCCATCAGCCGCGGCTT |
| | | | | | | 7875 | |
| | | | | | 2502 | 14753 | AGGACCTCAAATGTCTGCAGGTGTCCTCAAGGCTGGGGTTCATGGTGTCG |
| | | | | | | 722 | |
| | | | | | 2503 | 10333 | CAGGAGTGGGAGCCGCACGGCTTGGGGAACGCGGGAGATCGCGGATTGCG |
| | | | | | | 868 | |
| | | | | | 2504 | 60985 | CGCCCCATCCAGCCGCATCCCGGGACCCGGTCGCCAGGACCTCGCCGCTG |
| | | | | | | 732 | |
| | | | | | 2505 | 55107 | CGGCCCTCTCCTGGCAGCAAGCTCCCAGCGGCCAGTCTGAAGCCAATTCT |
| | | | | | | 755 | |
| | | | | | 2506 | 33402 | AGCAGGTGCCTCTCCGGGCGAGGCGCTATTGGCCGAGGGCTGGGGCGGCG |
| | | | | | | 512 | |
| | | | | | 2507 | 11708 | GAGCCTGTTTCAAACAGAGGCAGGCTGCTTCCTGAGGAACCAGCCCCTCG |
| | | | | | | 6941 | |
| | | | | | 2508 | 10289 | CGCCGGTTCTGGAACCAGGTTTTGACCTGCGCATCGGTCATTTTGAGCGC |
| | | | | | | 4120 | |
| | | | | | 2509 | 14263 | CGCAGGCCATGGGGAGCCCCGCTACCGTGAGCACCACCCACGCCGTCCCC |
| | | | | | | 2451 | |
| | | | | | 2510 | 17836 | GGGAGGTTAGTCCAGGGGAGCGACGCCCGATCGCTGACGAGAGAGGATCG |
| | | | | | | 7827 | |
| | | | | | 2511 | 24152 | ACAGTCACGTGGGACAGGGGCGTCCGAAGGTGCCGGCTCGGATGGGCTCG |
| | | | | | | 0598 | |
| | | | | | 2512 | 10111 | ACCTGAGCAGCGGTCACCACAGTAACGAATTCGCTGCCAGTGGAGTCTCG |
| | | | | | | 8525 | |
| | | | | | 2513 | 10834 | GTCCAAACCAAATTATCTCCCTGTGGTTCCTTGGGCTTCATCCGACAACG |
| | | | | | | 23 | |
| | | | | | 2514 | 14507 | CGCAGAAAGGACACAGTGACATGTAGTGTAAGTGACTGGCACCTTTCTGA |
| | | | | | | 8200 | |
| | | | | | 2515 | 10795 | ATTGCATCTTGCAACATTCGGAGCGTGGCCCCAGATACAGAGAACAGGCG |
| | | | | | | 6405 | |
| | | | | | 2516 | 84140 | GTGATCATAAGCCCATGGTGGGCAGAGGCCCTATGGAGCCAAGGGCTCCG |
| | | | | | | 997 | |
| | | | | | 2517 | 11953 | CGGTCCTAGCCAAGCTGAAAAAACGTCTCGGATTTCGCGGACAGCGGCCT |
| | | | | | | 2056 | |
| | | | | | 2518 | 91185 | CGCGCCAAAGGCATTTGATCCAACCCCGCTCCAGCCTACAGTGGCCCGAC |
| | | | | | | 422 | |
| | | | | | 2519 | 54723 | ATTTTTATTGCTGCCAGGGACCTCACCGGGGGCAGTGCTGGCACTGCTCG |
| | | | | | | 931 | |
| | | | | | 2520 | 27526 | CGATCTCGCCGTGGAGGCATGGATTCCAACCCTCCAGTGACGGTCGTATT |
| | | | | | | 023 | |
| | | | | | 2521 | 14034 | TCCAGATGAAAACCTACCTAATCCATCAAACAGTGTCTACTCTAAACACG |
| | | | | | | 2795 | |
| | | | | | 2522 | 14240 | TTTAATTTAAATTCAGAGTTAGCCGGAGCCATTTCCCCCCTCTAGAAGCG |
| | | | | | | 9993 | |
| | | | | | 2523 | 55370 | GAGCCCAGCTCCGGCTAGTTTTCCCGGGGGCAGGTGTAGCCTTGGGCGCG |
| | | | | | | 336 | |
| | | | | | 2524 | 12666 | GCTTGCCGAGTCCCAAGCAGGCAGGTGAGCTGGAGGGCGGGGATGTCCCG |
| | | | | | | 317 | |
| | | | | | 2525 | 11062 | ACCAGCACGTATGTTTTATCAGAATGGCCTTGAGAAAACGAAAGCACACG |
| | | | | | | 7473 | |
| | | | | | 2526 | 62362 | TGGGGCTGAGGCCTCGGGCTAGGGAAGGCGCCAAGGTGGATTCGGGCCCG |
| | | | | | | 783 | |
| | | | | | 2527 | 37173 | ATAGATGTGGACAAGAGAACATCTTTTACATCTCCACAGCTTTCAACTCG |
| | | | | | | 638 | |
| | | | | | 2528 | 21367 | CGGATGCGCGCCTTAACAGTTGTCCGTTTAGCAGGAGGTTTTGAGCTCCC |
| | | | | | | 368 | |
| | | | | | 2529 | 11638 | AAAGGAATCAGGGTATTTTGCTGGAAGGATGGCTGCCGAGGCCTACCACG |
| | | | | | | 1079 | |
| | | | | | 2530 | 81426 | CGAGTACCCCTTGCTTGTCGAGGCAACAAAGAGGAGCTTTTAAATGGAAG |
| | | | | | | 347 | |
| | | | | | 2531 | 60386 | AGTCTCGCTCTCTCCCATTCATTATCTTTAGCTCTCGGGTCCTGGAAGCG |
| | | | | | | 475 | |
| | | | | | 2532 | 28367 | GTGACCCACAAAGGCCGGAAGCGGCCACGGGGGGTCTAAGAACCAGCCCG |
| | | | | | | 571 | |
| | | | | | 2533 | 37249 | AGGAGAAGGCACCTAGGAGAAACGTCTTAGGGAGTAGTCCCGCGGGTCCG |
| | | | | | | 481 | |
| | | | | | 2534 | 27561 | GGTTTTCAAATGGGCCCCCGAGGCCTGGGAGATCCCCGAGTAGCCACGCG |
| | | | | | | 13 | |
| | | | | | 2535 | 15879 | CGACAGGAGTTACCATGTTTCAGGACAGCTTTCTGCCCTGATATGGTTTG |
| | | | | | | 9935 | |
| | | | | | 2536 | 46527 | ACTGTGGGGACTGCCTGCGAAACGCCTTGGAGGCCGCTGTGTGAGCTGCG |
| | | | | | | 098 | |
| | | | | | 2537 | 42727 | CGCCTGTGCCTCTCCAACTGCTTGGCCGTCTTCCGTCTCGGCCTCCTGCT |
| | | | | | | 489 | |
| | | | | | 2538 | 64887 | TTTGCTGATGGCTGAGGGCTTGGCCAGCCACCGGCCCGCCTGGGACGCCG |
| | | | | | | 827 | |
| | | | | | 2539 | 64875 | CGCACAGCTCCTCCGCAGGCTCGCCCAGGGCCAGCAGCAGCTCCACGCAC |
| | | | | | | 750 | |
| | | | | | 2540 | 17074 | CGGGGCACCCTGATCAGTTTCCTCAGGTTCAGCCAAGGCCTCTGTGGTTC |
| | | | | | | 6291 | |
| | | | | | 2541 | 65015 | CGGGATCCTAGGGAAGGCAGCCACAATGGAGATCCCTATCCACGGGAACG |
| | | | | | | 94 | |
| | | | | | 2542 | 19152 | CGCCACGGAAACCCAAACAGAACTCCGTGGGGAGCGAGGGTCTCACCTGC |
| | | | | | | 335 | |
| | | | | | 2543 | 36604 | CGCCTTACCCCAAAAGAACAGGCAAGAGAACTTCACCAATAGGTAAGGGC |
| | | | | | | 764 | |
| | | | | | 2544 | 51139 | AGGATTCACGGGCCTGTAGGGGCTCAGACTTCAAATTGAACCGCGCCTCG |
| | | | | | | 247 | |
| | | | | | 2545 | 11403 | TCATTTTAACGTGCGCAGGTCAGCGATGGGCTGAACGTTCCTTAAAATCG |
| | | | | | | 3360 | |
| | | | | | 2546 | 10289 | AAACCTGGTTCCAGAACCGGCGGACAAAGTGGAGGTGAGCAAGCGGGGCG |
| | | | | | | 4148 | |
| | | | | | 2547 | 97316 | AGTAGCCACCATTTGGTTAACAGACATGAAAACAAACCTTCTGACACTCG |
| | | | | | | 699 | |
| | | | | | 2548 | 73628 | CGCCTCCCCCACCTGCGCGAAGGCGAGGAGGTCCCACCTGCCTCAGCTGT |
| | | | | | | 634 | |
| | | | | | 2549 | 10487 | GGAGTGGGTGGCGGCGTCCGTGGGCGGCCGGGAGCCTGGCGTGGGTGGCG |
| | | | | | | 65 | |
| | | | | | 2550 | 11953 | CGCCCCCGGGACCCAGCTCTTTCCGGCTTCTTTGCAAAAGGATAGAACCG |
| | | | | | | 2195 | |
| | | | | | 2551 | 44326 | TGACAGCAGTGTTTCTTGAATAGGGTGACACTTCAGCCAGGGCATTTGCG |
| | | | | | | 516 | |
| | | | | | 2552 | 93516 | TATTACCTTAGCCATCTCCTTATTCCATGCTGACTGCTGAAGCCAAATCG |
| | | | | | | 589 | |
| | | | | | 2553 | 13611 | CGGCCACCCCGTGATTTTCTCTGAAAACTGCGACAGACTCGCAGCCTGCC |
| | | | | | | 400 | |
| | | | | | 2554 | 30383 | AACTTTGGACCAGCCTGTTTCACTCCAAAGCCTTTGGGGCTCAAGCTTCG |
| | | | | | | 960 | |
| | | | | | 2555 | 15557 | CGTGCTGAGAAAGGCTGTGACTAAATTAGTAAGATTTTCTTTAATTACTA |
| | | | | | | 0275 | |
| | | | | | 2556 | 19436 | CGCCAGGACTCACCCCGGCTCTCCACCTCCGCTGGGGGTTTCAGGTTCTG |
| | | | | | | 896 | |
| | | | | | 2557 | 75005 | CGGGTTCCACTAACTTTATCTCCTGAGGAATGCCTCAGGAATGCTACAAG |
| | | | | | | 362 | |
| | | | | | 2558 | 67391 | GTCTCCTTGCTACTGAGGCTCTCTTTCCTTTGATCCAGATGGTGTGGTCG |
| | | | | | | 835 | |
| | | | | | 2559 | 75315 | GGCAGGAGGAACGGCCGTGCCCCAGGCCAGCGTGCTGAGAAAAGGTTCCG |
| | | | | | | 667 | |
| | | | | | 2560 | 63446 | GGGATGGCAACTTGACACCACAAAGTATGGTTCTATTGGCTTAGCCAGCG |
| | | | | | | 044 | |
| | | | | | 2561 | 13428 | CGGTTGGTCCTTCACTGAGATCCCAAAGATCGAAGTCCTGGGCCTACTGC |
| | | | | | | 8122 | |
| | | | | | 2562 | 59729 | TTGTGCTTCCTCTCGGTGGGCAGGATTTCAGCGGAGTTGCGGGGGACACG |
| | | | | | | 553 | |
| | | | | | 2563 | | |
| | | | | | 2564 | 22818 | CGGAATATCCATAGAAAAAAATTCCAAGAATGTTTGGAGAGCAACGCATG |
| | | | | | | 572 | |
| | | | | | 2565 | 86233 | CGCTTCCTGGCATGGGGGTCGAATCATGTCGATAAAATGGGCTCGGTTTC |
| | | | | | | 236 | |
| | | | | | 2566 | 70681 | CGATGTGAAGCACATTCTGCGACTCCGCTGGAAACTCTTCAGCCACCCGT |
| | | | | | | 804 | |
| | | | | | 2567 | 13357 | CGACAGGTGAGAACAATGACAAGGGATCCACACTGTCAGGAATCTCATTA |
| | | | | | | 4073 | |
| | | | | | 2568 | 15114 | AAATCTCCGACGACGTGACGGCTGCTGCGGTGGGGAGCCGGGGAGGCTCG |
| | | | | | | 3302 | |
| | | | | | 2569 | 24803 | GCGGGGTGGTAGCGGCTTTGAGCGGGTGAGAAAAGCTCAGGTGGGGCCCG |
| | | | | | | 925 | |
| | | | | | 2570 | 17699 | CGCCCCTCAAGACCTGGCCTTAGCCTAATCTCAGGGTTTGCCCCACCCCG |
| | | | | | | 773 | |
| | | | | | 2571 | 15811 | CGGCATCCTTGGGCACGCACAGTAAGTTTGGCAGTTAAACACGTTAGCGG |
| | | | | | | 4605 | |
| | | | | | 2572 | 15402 | ATTTTAGCACCCTGCAAACAAGTCCTGAGAGGGCAAGCAAGCTGCTGACG |
| | | | | | | 7256 | |
| | | | | | 2573 | 11947 | CGTGATGCAGTATGGCCCCCGGGAGGCCGCCCCCAGATTCTAGAGGGGCT |
| | | | | | | 571 | |
| | | | | | 2574 | 77766 | CGTGGCCCCCGGCCCCTGCCCACCCACCCCCCAGGTCAGCGTCGCAAGAG |
| | | | | | | 120 | |
| | | | | | 2575 | 83960 | CGTGGATTTTGCAAAACCGTCATGTTCTCAAAAGGAGACCAACAGCTGAA |
| | | | | | | 56 | |
| | | | | | 2576 | 27999 | CGGGCATCTTACACTGTGAGTAGCTGTGGTTTTTTTCAAGATCGAAAGTT |
| | | | | | | 728 | |
| | | | | | 2577 | 21022 | CGGCCCAGAGTTTGACCCCTCCTAGCGAGTTCTTCCCGGCCAGCTGCCCA |
| | | | | | | 431 | |
| | | | | | 2578 | 43276 | GGGAGACAAAGGGCTATTTCGCTGTTACTTCAAAGAGCATTAAAGCCTCG |
| | | | | | | 744 | |
| | | | | | 2579 | 14709 | CATTCCTTTGTATGCGCGGGGCGCTGCGTGTCTGAGCTGGCGTCCTTGCG |
| | | | | | | 6130 | |
| | | | | | 2580 | 58554 | CGGGCTGCCCCGCGCAGAGCAGCTCCACCTGTCCCTCACCTACGCTCCCG |
| | | | | | | 527 | |
| | | | | | 2581 | 77595 | CTAAAACCTCACCAGAACCGCGCGGAAGTGAGAAGTCGCGGCCAAAGGCG |
| | | | | | | 359 | |
| | | | | | 2582 | 38035 | CGTGGAGGATGACATAGCAGTGTTCCTCAAAAAAGTTCCCGTAAGCCCCC |
| | | | | | | 463 | |
| | | | | | 2583 | 75315 | GACTCATGCGTGTGTGGTAGGAATCTTCCAGGAAGTCCCCGTCCCGTTCG |
| | | | | | | 567 | |
| | | | | | 2584 | 19184 | CGCAGCTCTCCGGATAGGCCGCCATCGCTTCGGCTTGGCCCTGCCTCTCA |
| | | | | | | 961 | |
| | | | | | 2585 | 74421 | CCTTTCCTGGTTATGGCCGGTAGCTGTCTCCAGGGCTAACGTGGGCAGCG |
| | | | | | | 918 | |
| | | | | | 2586 | 17702 | CGCAAAGGGCTCTCTCCGACTTGGAAAGTGCAGGGATCCCAAGAATATCA |
| | | | | | | 2056 | |
| | | | | | 2587 | 42277 | CGCCAGGACACCCCCCAAATGCGCGCCCTGGCACTCGTGCTGCGTCCTGT |
| | | | | | | 807 | |
| | | | | | 2588 | 13021 | TCACTCTTATCAGACTCCAAGAATTTAACCAAACCAGACAATCTAATGCG |
| | | | | | | 6901 | |
| | | | | | 2589 | 15128 | CAGGGAAGCCCTAGGGGGCCGGATGTGATGCTGCTGACTTGTGCATTGCG |
| | | | | | | 6477 | |
| | | | | | 2590 | 10539 | TTCGTCCTGATCACTCCATCTAAAAAACTGGTGTTCCCATAAGAATTACG |
| | | | | | | 8579 | |
| | | | | | 2591 | 62363 | CGCCGCGCCCTGGCCAAGCAACCCGCACACCACTGACTGGGGATCCGGGC |
| | | | | | | 076 | |
| | | | | | 2592 | 11953 | CGCCAGTCGTCCCCTGGATGCTCCTGGGCCGACTTTAACCCCTGGCTTCC |
| | | | | | | 1093 | |
| | | | | | 2593 | 18905 | AACTCCGAGATTGGTAACAAATGCCACAAATTCACTGTGTCCCCTGCTCG |
| | | | | | | 9677 | |
| | | | | | 2594 | 13540 | GGCGCCCGGGTCCGCAGGGTAGGTGGAGTCTGCGGTGGAAGATGGAGCCG |
| | | | | | | 946 | |
| | | | | | 2595 | 99947 | TAGTATTCTTTTATATTTCTTTCATTGGGCACTTTCCCATCTCCAACTCG |
| | | | | | | 171 | |
| | | | | | 2596 | 10849 | CTGGAGAGGGCCAGGCAAGGGCACAGCGGGTCTTGTCCTCCACTCCTTCG |
| | | | | | | 0895 | |
| | | | | | 2597 | 11138 | GCAGGGGCTGGTACCAACTTGGCGGTTCTCGGAGGGGCGGGGCAGGGGCG |
| | | | | | | 5461 | |
| | | | | | 2598 | 85673 | AATCCGAAGCCCAAGGAACCAGATGCATTGGCAACTGTTAATCGCAGCCG |
| | | | | | | 347 | |
| | | | | | 2599 | 75279 | CGGCATCTGTGCAGGAGGGTCTCCGCAGAAAAAACCCAGGACCCACGACC |
| | | | | | | 120 | |
| | | | | | 2600 | 85417 | GGTGTCCGCCACAGACTGAGCTTTCTTTCCCCACAAAACATTGTTTTTCG |
| | | | | | | 077 | |
| | | | | | 2601 | 37920 | CTCCCGCCTGGATCAAATTCAGGTGACTAAGCGCGTCTGCGAGGGAGGCG |
| | | | | | | 74 | |
| | | | | | 2602 | 17519 | CGCACCCTGGTCTGCTTGAGGACGCCTCTTTTCACACTGACAAGGTCCAG |
| | | | | | | 8280 | |
| | | | | | 2603 | 15726 | CGGATAATCCCGGTCCTGAGCCCCAGCGGCCAGTCTCCTTTACATATGAA |
| | | | | | | 166 | |
| | | | | | 2604 | 81166 | CGAAAGCCCGTCCACATCAACTCACCCTGGGCAATGACGTCTTCAATGTT |
| | | | | | | 9 | |
| | | | | | 2605 | 24323 | CGCTCGCCCGACAGCATAGTACTTGCCGCCCAGCCACGCCCGCGCGCCAG |
| | | | | | | 840 | |
| | | | | | 2606 | 53029 | CGGAGCCTGGGCTTTAAGGACTGGATGACCAGTGGGATCCAGTCTTCAAG |
| | | | | | | 709 | |
| | | | | | 2607 | 11637 | CGCGCCCCACCCTCAGCCGCCCCCAGGCAGCCTGCACGACCCAGCTCCTG |
| | | | | | | 1487 | |
| | | | | | 2608 | 13524 | CACCTGCGGAGGCCTGTGGCGAGTGCCGAGCTGAACCCACACCTGGAACG |
| | | | | | | 041 | |
| | | | | | 2609 | 41306 | CGGGGGATGAAGACACTGCTGCCATGGACAGCCCGTGCCAGCCGCAGCCC |
| | | | | | | 455 | |
| | | | | | 2610 | 13348 | CGCCCCAGCCCGTCCTGGCTGCCACCCGCCGTCCCTGAGAAACGCTCAGA |
| | | | | | | 4026 | |
| | | | | | 2611 | 86233 | TATCGACATGATTCGACCCCCATGCCAGGAAGCGTGCTCTCACAGAAGCG |
| | | | | | | 220 | |
| | | | | | 2612 | 46995 | TCCCCGGAGTTACAGTCGGAACAAACTTGCCGAGTCTTGCAGCGCAAGCG |
| | | | | | | 743 | |
| | | | | | 2613 | 12197 | CCTTATGAACCTGGAGGTATTGTGCTTCCCACATCACAGCAGCAGACACG |
| | | | | | | 2775 | |
| | | | | | 2614 | 81471 | CGGCCCATCTCTCAATTTTCAATCCAGCAGTTTGTACACGGCTGGTTTAA |
| | | | | | | 533 | |
| | | | | | 2615 | 23347 | ACTAGACCGACCATTAAATCTCCAGAAAAGGCCCAAGTCTAAAGTCTGCG |
| | | | | | | 412 | |
| | | | | | 2616 | 36990 | CTTAGCTGTTTTAGTCTCCTCTGCGCGCCAGGGGCTGGGAGGGCGGAGCG |
| | | | | | | 534 | |
| | | | | | 2617 | 22510 | CTCGAGTTCCGCGGATGGCCTGGAACCCGCAATTGGAGGAGTTGAGAGCG |
| | | | | | | 67 | |
| | | | | | 2618 | 15780 | CTTAGTCGCCAACCCACCTCATCTCAGGATGAATTTCAGTGTACTTGACG |
| | | | | | | 238 | |
| | | | | | 2619 | 15950 | CGCACACACAAGGGCTAAGGATGTACCAGAGAAGAGCATACGTAATTATG |
| | | | | | | 7162 | |
| | | | | | 2620 | 11103 | CCCCGACTGCTGGGCTGCCTCCTCGGGCAGGTGTGAGACTGCAGGGGACG |
| | | | | | | 9873 | |
| | | | | | 2621 | 11952 | GGTACCTCACAGACCCTTCTCCAAAGTCTGGCGGGCCTTGGGGTTTTTCG |
| | | | | | | 7377 | |
| | | | | | 2622 | 27153 | CGAAGTTTCTCAAACGAATTCCTAAGAAATAGAGCCCAGCAAGAGAGCCC |
| | | | | | | 944 | |
| | | | | | 2623 | 44640 | CGGGACAAGCTCTTCGGCTTCTGCCCGACCCGAGGTACCTCCACCCTGTC |
| | | | | | | 353 | |
| | | | | | 2624 | 23907 | CCGACAACGTCACTGTGATGGTGGTGCGGATAGGGCACTGAGGGGTGGCG |
| | | | | | | 9168 | |
| | | | | | 2625 | 21667 | CCTCTCAATTTCTGCATATTCATCCCTACATGAGTGCAGCTGGAAGAACG |
| | | | | | | 166 | |
| | | | | | 2626 | 77765 | GGGTCTCAAGACAGCTACGAGGGGCTGGCACCGGGAGGCTTTGGCTAACG |
| | | | | | | 514 | |
| | | | | | 2627 | 74024 | GGACCCAAGTTCAAGTTCCCTCCTCTCCAAACTCATCTGTTCTGGTCCCG |
| | | | | | | 572 | |
| | | | | | 2628 | 90357 | TCTCTGGCCAACACTGGGCTCAGAGGCCCTTTCTTTAACCTTAGATCTCG |
| | | | | | | 521 | |
| | | | | | 2629 | 19915 | TCCGAGGCAATTGTGTCAATGAGCTATTCAAAGACAAAAATAGTATGACG |
| | | | | | | 716 | |
| | | | | | 2630 | 10887 | CGCCCCCTGAAGTTTACTAGAGCTCTGTGTTTTGAGACTCAAGAAGCGCG |
| | | | | | | 185 | |
| | | | | | 2631 | 10152 | CGTGGTGGATTCGCTTTACTTATGACGAATCATTCACGGACAACACTTTT |
| | | | | | | 0662 | |
| | | | | | 2632 | 15890 | CGACACCAACTGGCAGATGATGTTGTGTTAACCCAGCAGCAGATCTGCAA |
| | | | | | | 0549 | |
| | | | | | 2633 | 13131 | CGGCCTCCATGCTTCTGGACACACACACGCTTCTCTGAAGGGCCAGCTGG |
| | | | | | | 9616 | |
| | | | | | 2634 | 20359 | CGCGCTAAATAACGACCTCACGGAGAAGCCGTCCCCGAGCCCACCAACAT |
| | | | | | | 8623 | |
| | | | | | 2635 | 31846 | CGGGATCCCCGGAGCCTGCCGTCCTTTGTTGAAATTACCCTCTGCCCAAT |
| | | | | | | 533 | |
| | | | | | 2636 | 64937 | CGCAAACTACAGCAGGATATGCGGAAAGCAACAAAAAGAGTCCCCCAGCA |
| | | | | | | 345 | |
| | | | | | 2637 | 26666 | TTGGAGGAGAGCGCAGGAGCGGAGGCTGCGGTGGGAGCCACTTTCCCCCG |
| | | | | | | 060 | |
| | | | | | 2638 | 81471 | CGCGGTCCCCGAGTGTGCGACAAATGGTACTCTTGCAACTTCCTGGGGCC |
| | | | | | | 884 | |
| | | | | | 2639 | 54507 | CGTGAGGTGACCCTCACCCAAGCCTTTCATGTGTTTGGCATGCTTAATTT |
| | | | | | | 223 | |
| | | | | | 2640 | 58554 | GCTAACACTGCCTGAGGCACCTCTGCTTTTGAAGTCGCCGGGGATTAGCG |
| | | | | | | 479 | |
| | | | | | 2641 | 97831 | CGCTGAGGGCGGTGAGCGAGGGCGCCAAGGCACAGGTGGGGCGGGAGTC |
| | | | | | | 92 | G |
| | | | | | 2642 | 85478 | CGGGCTCAGACCTGATTCTGATTCTTCTACCTGAGAACTGCGGAGTTGTT |
| | | | | | | 807 | |
| | | | | | 2643 | 13588 | CGCAGCCAGGTGGGGCACAGCTGATGCAAAAGGAGCTAACTAGGTGCAAA |
| | | | | | | 301 | |
| | | | | | 2644 | 28129 | GCTGGTTGCTGCTCACAGGCAGAGAAACGCTGGTATGCATTTGGGTGTCG |
| | | | | | | 616 | |
| | | | | | 2645 | 58716 | TATACATCACTACCGCATTCCCCCGAATAGGGGAGCCCCTCAGCATTTCG |
| | | | | | | 257 | |
| | | | | | 2646 | 93897 | GCCCGTGTCGCTACCATTCGGACAAAACGCACATCAAATTGCAGAATTCG |
| | | | | | | 195 | |
| | | | | | 2647 | 13092 | CGAAACCCTACGCCATTACAGAGTTCTAGTGTGGGCTACCTGTTGGGACA |
| | | | | | | 6603 | |
| | | | | | 2648 | 93779 | CGCGGAGCGAATGGTGTGCCAAGGAAGCAAGAAACTCCCACCAAAGCGAC |
| | | | | | | 84 | |
| | | | | | 2649 | 11381 | CGGAGGTAGCTGGACCGAAGCAAAAGATCGAGCCTCCGGTTTGGCTACTT |
| | | | | | | 7409 | |
| | | | | | 2650 | 13110 | CGCAGAGCACAGCGCTCCAGGTGCGCACTCGGGGCCAGCACTGTTCCCCA |
| | | | | | | 1463 | |
| | | | | | 2651 | 38964 | CGCTTTGTGTTGCTGGACCTTAAGAACTGACACACTTATTCACAGTCAGC |
| | | | | | | 993 | |
| | | | | | 2652 | 19915 | GACGTCATACTATTTTTGTCTTTGAATAGCTCATTGACACAATTGCCTCG |
| | | | | | | 762 | |
| | | | | | 2653 | 14708 | CGGCTCCCAGTGCGCGCGCATACCACATTTCCACAAACGTGTGTTACAAA |
| | | | | | | 7237 | |
| | | | | | 2654 | 22106 | CGGAAGACCCAGGACCTTCCTATGCTACACCCAAAGCCATTGAGAACGGG |
| | | | | | | 5480 | |
| | | | | | 2655 | 49297 | CGCGGTCCTCCACTGGGTAGGGGGCGCTCCAAGACAGGGACTCTTCTAGC |
| | | | | | | 822 | |
| | | | | | 2656 | 74707 | GTGGAAGTAGTTGGCCCCCCGGCCTCGAGCGGGGTGGATGAATAGCTCCG |
| | | | | | | 400 | |
| | | | | | 2657 | 66320 | CGGTCAGAAAGTTGAAGTTCCACAGCCACTGAGTTGGTAGGTACCACAGT |
| | | | | | | 20 | |
| | | | | | 2658 | 97326 | AATTTCCTGGTAGAGACAGGAGTTGTCCTGGGCCCCTGACATCCCAGCCG |
| | | | | | | 893 | |
| | | | | | 2659 | 89952 | CGGACCCCTGCGGGAAAACCAGACACTTCTGTTTCGTGGCTTTCGGGCTG |
| | | | | | | 904 | |
| | | | | | 2660 | 58207 | CGCCTAGGACCCGACACTGCAGCTGTCCGTTCTTGCCAGAGTCCCGGTCA |
| | | | | | | 859 | |
| | | | | | 2661 | 89560 | CGGGGCGCCTGGACCCCACTCGCCTCCATCTTGCTGCTGCTGCTTGGGCC |
| | | | | | | 715 | |
| | | | | | 2662 | 13356 | AGAGTTCCAGGCAATTCCGGGGGCTGCCCCACAGAAGCAGGTGGGGATCG |
| | | | | | | 2087 | |
| | | | | | 2663 | 45613 | GTCACCTGGGGAGTCGAAAATACTGAAATCCTGCGAAATTCCCTGTTGCG |
| | | | | | | 676 | |
| | | | | | 2664 | 10988 | CGTGCAGGCCCCGTCCACCCTCGGCACTGCCCGCACCCACGGCCCACCTG |
| | | | | | | 47 | |
| | | | | | 2665 | 10044 | CGCGAAGGCACCCCACGCTCCTAGAAAAGAGCACGACGCACCCGATGCTC |
| | | | | | | 2120 | |
| | | | | | 2666 | 10090 | GCCCGGGGAGTCGGCCGGCCAGGGAAGCTAGTCGGCTTGGTGTAGACCCG |
| | | | | | | 9238 | |
| | | | | | 2667 | 16906 | GGTTTGTTCAGTCCCTAGGCACGGCAATCAGGAAACTCTCTGCACTTGCG |
| | | | | | | 4024 | |
| | | | | | 2668 | 17073 | CTCGCGGCTCGGCGGGTCTAGGGACTTCGGGTTCCTGAATGTCAAAGGCG |
| | | | | | | 5347 | |
| | | | | | 2669 | 27265 | CGGGCCTATTCTCTACACACAAGTAGGGGCCGACAGGCTTGAGCGCCCTC |
| | | | | | | 522 | |
| | | | | | 2670 | 23083 | CGGTGGAGTTGAGGTGACATTTCACTGGCATGGAGACCTGAAACCAAAAG |
| | | | | | | 353 | |
| | | | | | 2671 | 32173 | CGGATATTACGGAAACACTTGGAAGCTGAACATCAGCCCGAGGACTCACT |
| | | | | | | 093 | |
| | | | | | 2672 | 50888 | CGCGCCTCCCGGCTCCACTGGCGACGCTGGAGCTCTTGGCCGCTTCCCTT |
| | | | | | | 826 | |
| | | | | | 2673 | 66638 | GGTGGGAGCTCCGAAGGGGTGCCGGGGCGGTGATCGGGAGGTCTCCAGCG |
| | | | | | | 433 | |
| | | | | | 2674 | 95939 | CGCCAAGGGTCAAGCACAGTGCTTGGTTCACAGAAAGGGGTTCAATATTT |
| | | | | | | 442 | |
| | | | | | 2675 | 54500 | GCGGAGGGTGGTGGGCTCCGAGTCCCGGGCGAGGGCGCGGAAGATGCTCG |
| | | | | | | 619 | |
| | | | | | 2676 | 33892 | ATTCAGCTGACAGTGTCCGCTTTCGGCGGGGTGTGGAATGGGCCAGAGCG |
| | | | | | | 223 | |
| | | | | | 2677 | 36253 | GAGGGGTACACACATTTCTGAAACTACTAAAATACTCACCTCCTGGGACG |
| | | | | | | 54 | |
| | | | | | 2678 | 10249 | GGCGGCGGGCGGCTCGGGTTATCTGAGCCGCTTGGTGTTGGGTGGCTGCG |
| | | | | | | 5354 | |
| | | | | | 2679 | 40680 | ATCAATAAAATGCTCACTGAAAACAAGTTACAAAAGCAATTTGGAGAGCG |
| | | | | | | 670 | |
| | | | | | 2680 | 80201 | AGGAACTCTTGAGGCCCTCGGAGAGCAGTTAGGGACCGCAGGCTGCCGCG |
| | | | | | | 887 | |
| | | | | | 2681 | 17275 | ACAATGCCTCCGGGCTGCGCGAGGTTGATAAGGGAGCAGCACCGAGCTCG |
| | | | | | | 1269 | |
| | | | | | 2682 | 65731 | CGGGGAACACGTGTAGCCTCCGGAGACCTAGGGGTCCATCGCTCCTCCCC |
| | | | | | | 432 | |
| | | | | | 2683 | 67874 | CGGAGTTCCGCAACTCTCCCCAGACGTCCGGAAGCAAATCGCGTCCCAGG |
| | | | | | | 206 | |
| | | | | | 2684 | 31376 | GCTCTCGGCTGCACTTGGGCAAGAGATAGCTGGAGCTATCGCCGCCTGCG |
| | | | | | | 092 | |
| | | | | | 2685 | 21022 | CGTCACCTGAGGGGGGCACTTCCGGTCTCGTGCCGCCTTCCCAGGACCCT |
| | | | | | | 877 | |
| | | | | | 2686 | 10231 | GGGAGCTGCAGAGCTACGCAGCCTTCGGTGCAGTCGTCACTCGTGTCTCG |
| | | | | | | 9088 | |
| | | | | | 2687 | 17719 | CGGGCGCTCCTGTGGTAGAGGGACTGGAACACTGTCCCATGTTGAGTGAA |
| | | | | | | 375 | |
| | | | | | 2688 | 86320 | CGCCCCTCGCCAGCGCGCTCGCAGCCCATCGGCTCCTGCCGGGGCCCCTC |
| | | | | | | 857 | |
| | | | | | 2689 | 92580 | CTTCCAAATCAAAGTTAACCAAAACAGCTTCAGAAGATGCCCATGGATCG |
| | | | | | | 100 | |
| | | | | | 2690 | 79171 | CGCCCCGGCCTCTCCTGATAGCACCTGGCACCTGCGTGAAGTATCCCTAC |
| | | | | | | 469 | |
| | | | | | 2691 | 13857 | CGTCAGTTTTTTTGAAAAGCCACTTTAGGAATAGACCAAAGGGGCTGGTG |
| | | | | | | 594 | |
| | | | | | 2692 | 10299 | TTTTCAAGTGCCTTAATTGGCTACTCCCCTCCGCGGGGAATTGGGCGCCG |
| | | | | | | 6254 | |
| | | | | | 2693 | 37387 | CGCGTTCATCCTGCTCCTCGCTCGCCAGCTCCCGTGCGGGGGCTCTAAGC |
| | | | | | | 577 | |
| | | | | | 2694 | 28832 | CGGACGTCTTTTGACTGGGTTCAGGTCACACTACTCCCAAAATACGACAC |
| | | | | | | 567 | |
| | | | | | 2695 | 43945 | CGGCCTTCTGCAGCGACTGGCGCTGGCTGCGCGCTCCCATCACAATCCCC |
| | | | | | | 364 | |
| | | | | | 2696 | 10090 | CGAGGGAAAATGAGACTTCCCCACAGGTTAATAAACAACCTGTCCGGCAC |
| | | | | | | 3839 | |
| | | | | | 2697 | 87682 | CAGGCCGCGTGAAGTGAGAAACGGTTTGCGGTGTCCTCCGCGGAGTGCCG |
| | | | | | | 142 | |
| | | | | | 2698 | 31637 | TGTCCCCTCTTACTTGTTCTGTCTTTCTCTCTCTCGCTTTAAAGGTGTCG |
| | | | | | | 348 | |
| | | | | | 2699 | 12195 | CGGTTCTTGCTCACGAGTTCCAAACAGTTAAAGTGACGTTGGCAAGGCCG |
| | | | | | | 0506 | |
| | | | | | 2700 | 15975 | CGGGGCTCCAGGCTGGTGCTTGTGCTGAGAATGACTGCGGGAGGGGCTCG |
| | | | | | | 6793 | |
| | | | | | 2701 | 56250 | GCTGCTGTGATCTTCCTGTCAAGCTGGTGACTGAGACTTCCCATCACTCG |
| | | | | | | 46 | |
| | | | | | 2702 | 20209 | TCTAGACACCAGTGGGCCGAGTGGAGCAGATGCCAGTTGGGACCTTCCCG |
| | | | | | | 8951 | |
| | | | | | 2703 | 17273 | CGCCTGCAGCCGGCGACTTTTCCCCAGGAATCTCTGGCCTGGCGCCCTTT |
| | | | | | | 4266 | |
| | | | | | 2704 | 21199 | CGGGAAGGCACTCCCTGACCGCAGATCACACTCTGCAGCTGATCCACACC |
| | | | | | | 691 | |
| | | | | | 2705 | 13626 | GGGCCTTGTCTCCCTAACGAGTGCCAGGTGAGGCTGCGGCGGCTCCGACG |
| | | | | | | 26 | |
| | | | | | 2706 | 13283 | CGCAGAGGGAGGAGAAGAGAAGGGGCAGAAACTAAACAGCCATAGTAGG |
| | | | | | | 794 | |
| | | | | | 2707 | 14572 | CTGGGGGAGATCCGCAAGTTAGCTTATGCGGTAGGGGGTGTCGCACTGCG |
| | | | | | | 2201 | |
| | | | | | 2708 | 43149 | TGGTAACGTGCAGTAGACGCACTGTATTCCATGGGCTCCCTTGTAAGCCG |
| | | | | | | 980 | |
| | | | | | 2709 | 32454 | CCTGGTTAGGCTAAGGTAGGAGCGGCCTGAAGACGCGCGTTTAGAAGGCG |
| | | | | | | 975 | |
| | | | | | 2710 | 79668 | GGCAGTTAGAGACGAGGCGTTTCAGGTGAACTGTATGTAGTGTTACTCCG |
| | | | | | | 668 | |
| | | | | | 2711 | 13724 | CGCCTGAAAACCAAACTGTCTGGCGCTTAACGGTTACCTCGGCTGAGCGA |
| | | | | | | 2062 | |
| | | | | | 2712 | 12619 | CAGAAACTGGAATGTGTGCCTAGCATGCCCCTAACCTGTATATGCACACG |
| | | | | | | 2611 | |
| | | | | | 2713 | 26156 | CTTTGCCTCTGTGGACCCACTGTGGAGGCAAGCAGGACATGAGATAATCG |
| | | | | | | 362 | |
| | | | | | 2714 | 47696 | CGGACACAGAGACTTGGTCATAAGGCAAACATTAACCCCCAGCGAACACG |
| | | | | | | 260 | |
| | | | | | 2715 | 62304 | AGACGGCCTTTCCCTAGGGTTCATAAAGACCCTTTCAAGCCACCCTCCCG |
| | | | | | | 515 | |
| | | | | | 2716 | 91184 | ACATTTTACTCCAAATTACCATTTTAAGAACCCTATGAATCTGCGAGGCG |
| | | | | | | 770 | |
| | | | | | 2717 | 12920 | GCCGCAGCAGCAGCGGCAGTCACAGGCACATCAGCAAAGAGGTACAGTCG |
| | | | | | | 4510 | |
| | | | | | 2718 | 21415 | CGCTATGGGTAGGGAAGCGGGGCTGCCTTGACTCTCAGTGCGGGTCCCCG |
| | | | | | | 3294 | |
| | | | | | 2719 | 17579 | ATATCGGGCGTCTTGCTTCAATTTCCTCCCGAGGTCTTGAGGGATGACCG |
| | | | | | | 2123 | |
| | | | | | 2720 | 30833 | CGGAGGGCCTGGTCGCCTGGAGGAAGCCGGAGGCCTGCGTGGAGGAGGCG |
| | | | | | | 38 | |
| | | | | | 2721 | 59085 | CGAAGACCTAGAGAAAGATGACAGCACGAAGAGAGACAGGGAGAATCCG |
| | | | | | | 561 | |
| | | | | | 2722 | 10090 | CCCTTGGGCGCTCTGGGGCCTACGGAGGCCTTAAACCCCAAAGATTGGCG |
| | | | | | | 3561 | |
| | | | | | 2723 | 10091 | AGCCAAGGTCCAGATCTGCCAAGGTAGGGAGCTGAAAGGCCCCTGCCTCG |
| | | | | | | 2168 | |
| | | | | | 2724 | 13702 | TTGAGTCAAAGGCAGGATCAGGTTCCCCGCCTTCCAGTCCAAAAATCCCG |
| | | | | | | 8389 | |
| | | | | | 2725 | 35395 | GGAGAATGTTCAGGACTGTCGAGGCGCGCTCCGCACCTGGCCTGAAAGCG |
| | | | | | | 973 | |
| | | | | | 2726 | 89951 | CGCGCACCCGTGGCAGCACCACAGAGGGAATAAAAGCAGGGTAAACAATT |
| | | | | | | 870 | |
| | | | | | 2727 | 15385 | CGGCGGCTCTGCGGGGTCCCAAGCCTCCCTGGCCTGGAGGCATTCTAGAA |
| | | | | | | 9740 | |
| | | | | | 2728 | 57244 | CGGACACTGGCCTCTTCGATCTCAGCACCGCAGTCAGAGGTGGTGGGGCA |
| | | | | | | 416 | |
| | | | | | 2729 | 13339 | GCGCGAGAGTGGGGTGGGTGACGCGCGCCAGCCACGGAGAATGAGGCTCG |
| | | | | | | 3454 | |
| | | | | | 2730 | 15358 | GAGTTGATTGCTATTGGGATCCGCTGAGTAAAGACACGGGCAGGGGTGCG |
| | | | | | | 4609 | |
| | | | | | 2731 | 20620 | ATGCAGGACCCATGCCTGCTTCAGTCATCGCAGAGCTGTGGGGCTATGCG |
| | | | | | | 680 | |
| | | | | | 2732 | 17305 | CTTCTGCTTGGGGTCCACCAGACGCTGCACATTTGCCACTCATCTGAACG |
| | | | | | | 61 | |
| | | | | | 2733 | 73163 | CGGTTCCTTGCAGTCCTCCGTGTCGTCCCGCACATCCTCATAGACAACGC |
| | | | | | | 696 | |
| | | | | | 2734 | 10111 | CCGCGAACTCCTGGCAGGTGAGCGAAGCTGCGGCCTTGGTGGGGGCAGCG |
| | | | | | | 1427 | |
| | | | | | 2735 | 39649 | TGCTGCAGGGTCGCGGTGGGGTTTGCGTCTGAAACGCCGGCGGTGGCTCG |
| | | | | | | 443 | |
| | | | | | 2736 | 13959 | CGGCCAGACCTCTCGACAGATCTCATTTTGTCGCGCCGTCTGGCCGCCAG |
| | | | | | | 3505 | |
| | | | | | 2737 | 62353 | CGCTCTGGGGCAGGATGGAGGTTGTGTCCTGGTCAGCCAACGGTTCTGTC |
| | | | | | | 894 | |
| | | | | | 2738 | 11342 | CGGCAGAACAAGAAGTGGCTGTGTCACTGGATACCTGTACATTTGAATAC |
| | | | | | | 5199 | |
| | | | | | 2739 | 20033 | TCCCCCTTTTTCGCCACGCGGAAACTGGCCGGTCTGGACAAGGGATTGCG |
| | | | | | | 2025 | |
| | | | | | 2740 | 10005 | CTAAATGTGCCCAACAGCAAGGAAAATCGAATTGAGTGCGGGCTCATTCG |
| | | | | | | 0791 | |
| | | | | | 2741 | 76746 | CGGCTCTGACCACACACTCCCGCGCGTCGACGCCATGGCCCACACCTCAA |
| | | | | | | 337 | |
| | | | | | 2742 | 62070 | CGCCCGCCCTGCGTCTCCGCACCAAGCCCTGTAGCTCCGAGAGGCATGAA |
| | | | | | | 164 | |
| | | | | | 2743 | 15848 | CGGCTCGGAAGCCGAAGAATCGAAACATGGCTCAAGAAGCTACTTGTCAC |
| | | | | | | 8659 | |
| | | | | | 2744 | 12463 | ACACTGGAATGTTGGCCCAGTGTATGACTCAGGCTAGTTCCCAGCAGTCG |
| | | | | | | 3001 | |
| | | | | | 2745 | 12903 | CGGAAGCAGGTTCAGGATGACAGAGCCAACCCCATGATTACAGACTGAAT |
| | | | | | | 5348 | |
| | | | | | 2746 | 84140 | AGGGGCCCGAGCGAAGCCGGTGTCCCCAGCAGGGCAGCCTGCGAGAAGCG |
| | | | | | | 898 | |
| | | | | | 2747 | 10271 | CGCCCTGGCCTCAGTGCCCGAGTGCAGTTGTCTAACGATGGCCCGTGGTC |
| | | | | | | 2216 | |
| | | | | | 2748 | 10090 | CACTTTGAGAGGGATCCCCTATGGTATCCAGTCAAAGGGGCCGGCACTCG |
| | | | | | | 1804 | |
| | | | | | 2749 | 17714 | CGCACGGCGGCGACACACGGGAGAAGCGAGCATGTTCCCAACATATATAC |
| | | | | | | 293 | |
| | | | | | 2750 | 13136 | TCTCGGAGCCTGGAAGACATTATGACCACTAAGCGGGTGTCCATAATTCG |
| | | | | | | 9037 | |
| | | | | | 2751 | 19788 | CGCCTTCGTTGGGTGGAACGAAGAGGAGTGTGTGTTTGTCTTAAAAATTA |
| | | | | | | 1774 | |
| | | | | | 2752 | 57004 | AGCAACAGTGATTCTTAGAGAAGTAACATTAACACATCACAGAGCATCCG |
| | | | | | | 1 | |
| | | | | | 2753 | 19585 | CGCCCCTGGACACCGCCCAGAAAGGACTCACAGCCAAGTGGCAGGCCAGG |
| | | | | | | 665 | |
| | | | | | 2754 | 10091 | GGCCAGGGGCGAGGCGCTCATTGGCCAATAGGGCTGAGTGACACGAGTCG |
| | | | | | | 2940 | |
| | | | | | 2755 | 11953 | CGGGGCAGGCGCTCACAGACTGTGTCCACTGAACTTCATCTTGTTTTTGT |
| | | | | | | 0702 | |
| | | | | | 2756 | 11954 | CGCCCCCACGGGACTCAGAACCCTGAGGCTCTTCTAGAGACACCAACTTG |
| | | | | | | 2629 | |
| | | | | | 2757 | 22106 | GTGGTCACCATCCCTCTGTGAGCCTCTGTGCACCTTGGTAGCCTTTTTCG |
| | | | | | | 1070 | |
| | | | | | 2758 | 45285 | TCTTGAGGGTATCTTTTCGTGGTGCACCATGGGTATGCAAATCACAGGCG |
| | | | | | | 892 | |
| | | | | | 2759 | 79574 | ACTCGCTCAGCAACTTAGCAGGGGGCTCCTAGACTCCCCGAGGGTGAGCG |
| | | | | | | 893 | |
| | | | | | 2760 | 10823 | CGGGGCCTGCACGGAAGGACAGGGGCGCCTCCCAGACCCACCCTTCCCGC |
| | | | | | | 7150 | |
| | | | | | 2761 | 22363 | ATCACAAAAGAATTAATGTTTTCACAGCCCTTTGAAGACGTGGTGTCACG |
| | | | | | | 174 | |
| | | | | | 2762 | 13663 | CGGCGCGAGGGGTTTCATTTGTCTCAGTGTTGCAAGTTTACAGGTGCTGA |
| | | | | | | 0265 | |
| | | | | | 2763 | 65331 | GCAAGAGTTTGCAAAAACATTGGGCGTCTCATTTGGCGAGCGTTTTGGCG |
| | | | | | | 87 | |
| | | | | | 2764 | 69706 | AGAATGCCCTGCCGGAGATATAGGATAGCAGTATCCCGTGGGGTACGCCG |
| | | | | | | 527 | |
| | | | | | 2765 | 16228 | CGCAGCAGATGGTGGCGGAGCTGCAAGGGAAACGACCTCCCCAATTTTTT |
| | | | | | | 3994 | |
| | | | | | 2766 | 85417 | CGAAAGCCAAGTGGAGGTTCTCTTGGGAAGCCCGGGGACTGTGGCAGCCG |
| | | | | | | 612 | |
| | | | | | 2767 | 64008 | CGAGGGATTTGCACAAGGCTTCATAGTAAGGAGCCATGTCTAACATGCAA |
| | | | | | | 161 | |
| | | | | | 2768 | 67803 | CGGCATGTCCGGATGCTCGTTTCCCAGTAAGATTGTATTAGAAGGCAGTT |
| | | | | | | 251 | |
| | | | | | 2769 | 23350 | GTGTGGCAGGGGAATTTCTAGCCCCTTCCAATGTGTCCCTCCCAGGGACG |
| | | | | | | 651 | |
| | | | | | 2770 | 26797 | GAGGTCGTCGTCGTCGCCCAGCAGCAAGGGCTGCAAAATAGTAGAACTCG |
| | | | | | | 619 | |
| | | | | | 2771 | 14304 | CTCAGGCAGGGCGTGGGTTTCCCTCAGATTCAATGATCCTGCTGGGCTCG |
| | | | | | | 2635 | |
| | | | | | 2772 | 12667 | AATTCGCTCCTGCAGTCCCTAAACTTGACTGTAACCACCTCTGGTTTTCG |
| | | | | | | 5667 | |
| | | | | | 2773 | 42543 | CGCCCCTTGGAAGCTCAGCACCAAAGGCAGGCGGCCAGGGTCCTGGCCCC |
| | | | | | | 161 | |
| | | | | | 2774 | 65360 | CGGGCGGGGCCACCCCAAGCGCCACCAAATTCCTTGTAAGGGCTGCGGAG |
| | | | | | | 366 | |
| | | | | | 2775 | 11671 | CGGGAGCACCCCGCCAGGGGACCGGGAAGGGTCGAGGAAAACTGCTGCCG |
| | | | | | | 1075 | |
| | | | | | 2776 | 10100 | TAAGCCGTCAAAGGGGGCGTGGGGCTTTTTTGTTAACCCTCGGCGGTCCG |
| | | | | | | 6058 | |
| | | | | | 2777 | 29438 | GGCAGTTTTTGTGTCTAATCACTGCCATAGATGGAGCTTCTGGGCTCTCG |
| | | | | | | 91 | |
| | | | | | 2778 | 22298 | CGGAAAATCAGGAGTCATATGAGGAAGGCTGGGAAATGAATTCTCTTGTA |
| | | | | | | 146 | |
| | | | | | 2779 | 74425 | CAGATGCACGCTGGGCGGTGCACAGGGCAGGGCGGGCAGGCGGTACACCG |
| | | | | | | 757 | |
| | | | | | 2780 | 22094 | CGGCACTTAGCTTTCGCGATCGCTATTTCTAGAATTAAGGGACGAGGCCC |
| | | | | | | 009 | |
| | | | | | 2781 | 57125 | CGCCTAATTTTCCGCACGGATGAGTAGACCCAGGCCCAGTTCGGATCCGT |
| | | | | | | 836 | |
| | | | | | 2782 | 62360 | CGCGGTGCTTTAGTGGCTAAAAGTGAAAGAGCTGCCTTGGCAGAATTCAG |
| | | | | | | 162 | |
| | | | | | 2783 | 55125 | CGGGCAGGCTGTGAAGCATCCAACACAAACCGAAGCCGCCCGCCTGGCGT |
| | | | | | | 644 | |
| | | | | | 2784 | 38677 | TTGGGCGGGCGGGGACGCCAGCTCTTCTAAATCCAGGTGAGGAGCTGCCG |
| | | | | | | 218 | |
| | | | | | 2785 | 20692 | CGCTGAGAGTCCTCACAACTGCGTCTCCGGAGGCAGAACGAAGTGGGGAC |
| | | | | | | 113 | |
| | | | | | 2786 | 16344 | CGGCAGATTGTGTAGTGGTGAACCTATGGTTTTCTGAAGATATCTCTTAC |
| | | | | | | 574 | |
| | | | | | 2787 | 14539 | CGGAAAAAATTACAATGGATAAGGACACAGTAGGCTTTTTGAGTATCCAG |
| | | | | | | 5986 | |
| | | | | | 2788 | 15725 | CGAGAGCAATGCGTATACAGCCAGCAGCTCTAAGCCCTCCAAAGTCCTAA |
| | | | | | | 322 | |
| | | | | | 2789 | 21666 | GAGCTTAGTGTGGGGCTGCCCGGGCTGGTCCCCAGCTCACCGCAGCTGCG |
| | | | | | | 098 | |
| | | | | | 2789 | | CGAAGGGCCTGAGGGGTCCAACTGCGCATGTGTATTCCTCGGTTTTCCCG |
| | | | | | 2789 | | TAGTTTCAGAAGAATGTCCAGGGCCTTTCCACCACCCTGGGGGCAGCACG |
| | | | | | 2789 | | TCTGAGTAAGGAAACAGCCTCCAAGCATCAGCAGAGCCCAGATGAGCACG |
| | | | | | 2789 | | GTTGGGAAGGACTTAACTTTTCACCGCAGGGCCTGAGTCCTGGCTTTCCG |
| | | | | | 2789 | | GAGGGGCTCAGGCTGCGGGAGCGCCGCTGCAGGAGCCTGGGCGGGCTGCG |
| | | | | | 2789 | | CGGGGCACAATGATCTGTCCTTGAGGACAAAGAGTGATTCCGGAGAGAGC |
| | | | | | 2789 | | CGGGCCTGGCCCAATTACTTTCTTCAGTCTGAGCATGAGGGCTGGAAGAA |
| | | | | | 2789 | | CGCTACCCTTGTGCTTGCGTTGCCCACAAGCTTGTAGTCAGAAGAACTCG |
| | | | | | 2748 | 10090 | CACTTTGAGAGGGATCCCCTATGGTATCCAGTCAAAGGGGCCGGCACTCG |
| | | | | | | 1804 | |
| | | | | | 2749 | 17714 | CGCACGGCGGCGACACACGGGAGAAGCGAGCATGTTCCCAACATATATAC |
| | | | | | | 293 | |
| | | | | | 2750 | 13136 | TCTCGGAGCCTGGAAGACATTATGACCACTAAGCGGGTGTCCATAATTCG |
| | | | | | | 9037 | |
| | | | | | 2751 | 19788 | CGCCTTCGTTGGGTGGAACGAAGAGGAGTGTGTGTTTGTCTTAAAAATTA |
| | | | | | | 1774 | |
| | | | | | 2752 | 57004 | AGCAACAGTGATTCTTAGAGAAGTAACATTAACACATCACAGAGCATCCG |
| | | | | | | 1 | |
| | | | | | 2753 | 19585 | CGCCCCTGGACACCGCCCAGAAAGGACTCACAGCCAAGTGGCAGGCCAGG |
| | | | | | | 665 | |
| | | | | | 2754 | 10091 | GGCCAGGGGCGAGGCGCTCATTGGCCAATAGGGCTGAGTGACACGAGTCG |
| | | | | | | 2940 | |
| | | | | | 2755 | 11953 | CGGGGCAGGCGCTCACAGACTGTGTCCACTGAACTTCATCTTGTTTTTGT |
| | | | | | | 0702 | |
| | | | | | 2756 | 11954 | CGCCCCCACGGGACTCAGAACCCTGAGGCTCTTCTAGAGACACCAACTTG |
| | | | | | | 2629 | |
| | | | | | 2757 | 22106 | GTGGTCACCATCCCTCTGTGAGCCTCTGTGCACCTTGGTAGCCTTTTTCG |
| | | | | | | 1070 | |
| | | | | | 2758 | 45285 | TCTTGAGGGTATCTTTTCGTGGTGCACCATGGGTATGCAAATCACAGGCG |
| | | | | | | 892 | |
| | | | | | 2759 | 79574 | ACTCGCTCAGCAACTTAGCAGGGGGCTCCTAGACTCCCCGAGGGTGAGCG |
| | | | | | | 893 | |
| | | | | | 2760 | 10823 | CGGGGCCTGCACGGAAGGACAGGGGCGCCTCCCAGACCCACCCTTCCCGC |
| | | | | | | 7150 | |
| | | | | | 2761 | 22363 | ATCACAAAAGAATTAATGTTTTCACAGCCCTTTGAAGACGTGGTGTCACG |
| | | | | | | 174 | |
| | | | | | 2762 | 13663 | CGGCGCGAGGGGTTTCATTTGTCTCAGTGTTGCAAGTTTACAGGTGCTGA |
| | | | | | | 0265 | |
| | | | | | 2763 | 65331 | GCAAGAGTTTGCAAAAACATTGGGCGTCTCATTTGGCGAGCGTTTTGGCG |
| | | | | | | 87 | |
| | | | | | 2764 | 69706 | AGAATGCCCTGCCGGAGATATAGGATAGCAGTATCCCGTGGGGTACGCCG |
| | | | | | | 527 | |
| | | | | | 2765 | 16228 | CGCAGCAGATGGTGGCGGAGCTGCAAGGGAAACGACCTCCCCAATTTTTT |
| | | | | | | 3994 | |
| | | | | | 2766 | 85417 | CGAAAGCCAAGTGGAGGTTCTCTTGGGAAGCCCGGGGACTGTGGCAGCCG |
| | | | | | | 612 | |
| | | | | | 2767 | 64008 | CGAGGGATTTGCACAAGGCTTCATAGTAAGGAGCCATGTCTAACATGCAA |
| | | | | | | 161 | |
| | | | | | 2768 | 67803 | CGGCATGTCCGGATGCTCGTTTCCCAGTAAGATTGTATTAGAAGGCAGTT |
| | | | | | | 251 | |
| | | | | | 2769 | 23350 | GTGTGGCAGGGGAATTTCTAGCCCCTTCCAATGTGTCCCTCCCAGGGACG |
| | | | | | | 651 | |
| | | | | | 2770 | 26797 | GAGGTCGTCGTCGTCGCCCAGCAGCAAGGGCTGCAAAATAGTAGAACTCG |
| | | | | | | 619 | |
| | | | | | 2771 | 14304 | CTCAGGCAGGGCGTGGGTTTCCCTCAGATTCAATGATCCTGCTGGGCTCG |
| | | | | | | 2635 | |
| | | | | | 2772 | 12667 | AATTCGCTCCTGCAGTCCCTAAACTTGACTGTAACCACCTCTGGTTTTCG |
| | | | | | | 5667 | |
| | | | | | 2773 | 42543 | CGCCCCTTGGAAGCTCAGCACCAAAGGCAGGCGGCCAGGGTCCTGGCCCC |
| | | | | | | 161 | |
| | | | | | 2774 | 65360 | CGGGCGGGGCCACCCCAAGCGCCACCAAATTCCTTGTAAGGGCTGCGGAG |
| | | | | | | 366 | |
| | | | | | 2775 | 11671 | CGGGAGCACCCCGCCAGGGGACCGGGAAGGGTCGAGGAAAACTGCTGCCG |
| | | | | | | 1075 | |
| | | | | | 2776 | 10100 | TAAGCCGTCAAAGGGGGCGTGGGGCTTTTTTGTTAACCCTCGGCGGTCCG |
| | | | | | | 6058 | |
| | | | | | 2777 | 29438 | GGCAGTTTTTGTGTCTAATCACTGCCATAGATGGAGCTTCTGGGCTCTCG |
| | | | | | | 91 | |
| | | | | | 2778 | 22298 | CGGAAAATCAGGAGTCATATGAGGAAGGCTGGGAAATGAATTCTCTTGTA |
| | | | | | | 146 | |
| | | | | | 2779 | 74425 | CAGATGCACGCTGGGCGGTGCACAGGGCAGGGCGGGCAGGCGGTACACCG |
| | | | | | | 757 | |
| | | | | | 2780 | 22094 | CGGCACTTAGCTTTCGCGATCGCTATTTCTAGAATTAAGGGACGAGGCCC |
| | | | | | | 009 | |
| | | | | | 2781 | 57125 | CGCCTAATTTTCCGCACGGATGAGTAGACCCAGGCCCAGTTCGGATCCGT |
| | | | | | | 836 | |
| | | | | | 2782 | 62360 | CGCGGTGCTTTAGTGGCTAAAAGTGAAAGAGCTGCCTTGGCAGAATTCAG |
| | | | | | | 162 | |
| | | | | | 2783 | 55125 | CGGGCAGGCTGTGAAGCATCCAACACAAACCGAAGCCGCCCGCCTGGCGT |
| | | | | | | 644 | |
| | | | | | 2784 | 38677 | TTGGGCGGGCGGGGACGCCAGCTCTTCTAAATCCAGGTGAGGAGCTGCCG |
| | | | | | | 218 | |
| | | | | | 2785 | 20692 | CGCTGAGAGTCCTCACAACTGCGTCTCCGGAGGCAGAACGAAGTGGGGAC |
| | | | | | | 113 | |
| | | | | | 2786 | 16344 | CGGCAGATTGTGTAGTGGTGAACCTATGGTTTTCTGAAGATATCTCTTAC |
| | | | | | | 574 | |
| | | | | | 2787 | 14539 | CGGAAAAAATTACAATGGATAAGGACACAGTAGGCTTTTTGAGTATCCAG |
| | | | | | | 5986 | |
| | | | | | 2788 | 15725 | CGAGAGCAATGCGTATACAGCCAGCAGCTCTAAGCCCTCCAAAGTCCTAA |
| | | | | | | 322 | |
| | | | | | 2789 | 21666 | GAGCTTAGTGTGGGGCTGCCCGGGCTGGTCCCCAGCTCACCGCAGCTGCG |
| | | | | | | 098 | |
| | | | | | 2789 | | CGAAGGGCCTGAGGGGTCCAACTGCGCATGTGTATTCCTCGGTTTTCCCG |
| | | | | | 2789 | | TAGTTTCAGAAGAATGTCCAGGGCCTTTCCACCACCCTGGGGGCAGCACG |
| | | | | | 2789 | | TCTGAGTAAGGAAACAGCCTCCAAGCATCAGCAGAGCCCAGATGAGCACG |
| | | | | | 2789 | | GTTGGGAAGGACTTAACTTTTCACCGCAGGGCCTGAGTCCTGGCTTTCCG |
| | | | | | 2789 | | |
| | | | | | NM_1 | | |
| | | | | | 72056; | | |
| | | | | | NM_ | | |
| | | | | | 000238 | | |
| | | | | | 2789 | | CGGGGCACAATGATCTGTCCTTGAGGACAAAGAGTGATTCCGGAGAGAGC |
| | | | | | 2789 | | CGGGCCTGGCCCAATTACTTTCTTCAGTCTGAGCATGAGGGCTGGAAGAA |
| | | | | | 2789 | | CGCTACCCTTGTGCTTGCGTTGCCCACAAGCTTGTAGTCAGAAGAACTCG |
| | | | | | 2787 | 14539 | CGGAAAAAATTACAATGGATAAGGACACAGTAGGCTTTTTGAGTATCCAG |
| | | | | | | 5986 | |
| | | | | | 2788 | 15725 | CGAGAGCAATGCGTATACAGCCAGCAGCTCTAAGCCCTCCAAAGTCCTAA |
| | | | | | | 322 | |
| | | | | | 2789 | 21666 | GAGCTTAGTGTGGGGCTGCCCGGGCTGGTCCCCAGCTCACCGCAGCTGCG |
| | | | | | | 098 | |
| | | | | | 2789 | | CGAAGGGCCTGAGGGGTCCAACTGCGCATGTGTATTCCTCGGTTTTCCCG |
| | | | | | 2789 | | TAGTTTCAGAAGAATGTCCAGGGCCTTTCCACCACCCTGGGGGCAGCACG |
| | | | | | 2789 | | TCTGAGTAAGGAAACAGCCTCCAAGCATCAGCAGAGCCCAGATGAGCACG |
| | | | | | 2789 | | GTTGGGAAGGACTTAACTTTTCACCGCAGGGCCTGAGTCCTGGCTTTCCG |
| | | | | | 2789 | | GAGGGGCTCAGGCTGCGGGAGCGCCGCTGCAGGAGCCTGGGCGGGCTGCG |
| | | | | | 2789 | | CGGGGCACAATGATCTGTCCTTGAGGACAAAGAGTGATTCCGGAGAGAGC |
| | | | | | 2789 | | CGGGCCTGGCCCAATTACTTTCTTCAGTCTGAGCATGAGGGCTGGAAGAA |
| | | | | | 2789 | | CGCTACCCTTGTGCTTGCGTTGCCCACAAGCTTGTAGTCAGAAGAACTCG |

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are each relied upon and incorporated herein in their entirety.
1. Prensner, J. R., Rubin, M. A., Wei, J. T. & Chinnaiyan, A. M. Beyond PSA: the next generation of prostate cancer biomarkers. Sci. Transl. Med. 4, 127rv3 (2012).
2. Ahmed, H. U. et al. Focal therapy for localised unifocal and multifocal prostate cancer: a prospective development study. Lancet Oncol. 13, 622-32 (2012).
3. Gilbert, S. M. et al. Quality of life and satisfaction among prostate cancer patients followed in a dedicated survivorship clinic. Cancer 121, 1484-91 (2015).
4. Johnson, L. M., Turkbey, B., Figg, W. D. & Choyke, P. L. Multiparametric MRI in prostate cancer management. Nat. Rev. Clin. Oncol. 11, 346-53 (2014).
5. Gleason, D. F. Histologic grading of prostate cancer: A perspective. Hum. Pathol. 23, 273-279 (1992).
6. Humphrey, P. A. Gleason grading and prognostic factors in carcinoma of the prostate. Mod. Pathol. 17, 292-306 (2004).
7. Tomlins, S. A. et al. Integrative molecular concept modeling of prostate cancer progression. Nat. Genet. 39, 41-51 (2007).
8. Andreoiu, M. & Cheng, L. Multifocal prostate cancer: biologic, prognostic, and therapeutic implications. Hum. Pathol. 41, 781-93 (2010).
9. Barry, M., Perner, S., Demichelis, F. & Rubin, M. A. TMPRSS2-ERG fusion heterogeneity in multifocal prostate cancer: clinical and biologic implications. Urology 70, 630-3 (2007).
10. Boyd, L. K. et al. High-Resolution Genome-Wide Copy-Number Analysis Suggests a Monoclonal Origin of Multifocal Prostate Cancer. Genes Chromosom. cancer 51, 579-589 (2012).
11. Brocks, D. et al. Intratumor DNA methylation heterogeneity reflects clonal evolution in aggressive prostate cancer. Cell Rep. 8, 798-806 (2014).
12. Cooper, C. S. et al. Analysis of the genetic phylogeny of multifocal prostate cancer identifies multiple independent clonal expansions in neoplastic and morphologically normal prostate tissue. Nat. Genet. 47, (2015).
13. Haffner, M. C. et al. Tracking the clonal origin of lethal prostate cancer. J. Clin. Invest. 123, 4918-22 (2013).
14. Konishi, N. et al. Heterogeneous methylation and deletion patterns of the INK4a/ARF locus within prostate carcinomas. Am. J. Pathol. 160, 1207-14 (2002).
15. Kovtun, I. V et al. Lineage relationship of Gleason patterns in Gleason score 7 prostate cancer. Cancer Res. 73, 3275-84 (2013).
16. Lindberg, J. et al. Exome sequencing of prostate cancer supports the hypothesis of independent tumour origins. Eur. Urol. 63, 347-53 (2013).
17. Woodson, K. et al. Heterogeneous gene methylation patterns among pre-invasive and cancerous lesions of the prostate: a histopathologic study of whole mount prostate specimens. Prostate 60, 25-31 (2004).
18. Schmidt, H. et al. Asynchronous growth of prostate cancer is reflected by circulating tumor cells delivered from distinct, even small foci, harboring loss of heterozygosity of the PTEN gene. Cancer Res. 66, 8959-65 (2006).
19. Arora, R. et al. Heterogeneity of Gleason grade in multifocal adenocarcinoma of the prostate. Cancer 100, 2362-6 (2004).
20. Ruijter, E. T., van de Kaa, C. A., Schalken, J. A., Debruyne, F. M. & Ruiter, D. J. Histological grade heterogeneity in multifocal prostate cancer. Biological and clinical implications. J. Pathol. 180, 295-9 (1996).
21. Jones, P. A. & Baylin, S. B. The epigenomics of cancer. Cell 128, 683-92 (2007).
22. Chiam, K., Ricciardelli, C. & Bianco-Miotto, T. Epigenetic biomarkers in prostate cancer: Current and future uses. Cancer Lett. 342, 248-56 (2014).
23. Strand, S., Orntoft, T. & Sorensen, K. Prognostic DNA Methylation Markers for Prostate Cancer. Int. J. Mol. Sci. 15, 16544-16576 (2014).
24. Hemminki, K. Familial risk and familial survival in prostate cancer. World J. Urol. 30, 143-148 (2012).
25. Lindstrom, L. S. et al. Familial concordance in cancer survival: a Swedish population-based study. Lancet Oncol. 8, 1001-1006 (2007).
26. Al Olama, A. A. et al. A meta-analysis of 87,040 individuals identifies 23 new susceptibility loci for prostate cancer. Nat. Genet. 46, 1103-1109 (2014).
27. Liu, W. et al. Copy number analysis indicates monoclonal origin of lethal metastatic prostate cancer. Nat. Med. 15, 559-65 (2009).
28. Aryee, M. J. et al. DNA Methylation Alterations Exhibit Intraindividual Stability and Interindividual Heterogeneity in Prostate Cancer Metastases. Sci. Transl. Med. 5, 169ra10 (2013).
29. Mazor, T. et al. DNA Methylation and Somatic Mutations Converge on the Cell Cycle and Define Similar Evolutionary Histories in Brain Tumors Article DNA Methylation and Somatic Mutations Converge on the Cell Cycle and Define Similar Evolutionary Histories in Brain. Cancer Cell 28, 307-317 (2015).
30. Sturm, D. et al. Hotspot Mutations in H3F3A and IDH1 Define Distinct Epigenetic and Biological Subgroups of Glioblastoma. Cancer Cell 22, 425-437 (2012).
31. Feber, A. et al. Using high-density DNA methylation arrays to profile copy number alterations. Genome Biol. 15, R30 (2014).
32. Kim, T. et al. Functional genomic analysis of chromosomal aberrations in a compendium of 8000 cancer genomes. Genome Res. 23, 217-227 (2013).
33. Boutros, P. C. et al. Spatial genomic heterogeneity within localized, multifocal prostate cancer. Nat. Genet. 47, 736-745 (2015).
34. Pereira, V., Waxman, D. & Eyre-Walker, A. A problem with the correlation coefficient as a measure of gene expression divergence. Genetics 183, 1597-1600 (2009).
35. Friedman, J., Hastie, T. & Tibshirani, R. Regularization Paths for Generelized Linear Models via Coordinate Descent. J. Stat. Softw. 33, 1-22 (2010).
36. Hambrock, T. et al. Prospective assessment of prostate cancer aggressiveness using 3-T diffusion-weighted magnetic resonance imaging-guided biopsies versus a systematic 10-core transrectal ultrasound prostate biopsy cohort. Eur. Urol. 61, 177-184 (2012).
37. Pokorny, M. R. et al. Prospective study of diagnostic accuracy comparing prostate cancer detection by transrectal ultrasound-guided biopsy versus magnetic resonance (MR) imaging with subsequent mr-guided biopsy in men without previous prostate biopsies. Eur. Urol. 66, 22-29 (2014).
38. Ukimura, O. & Gill, I. S. Targeted prostate biopsies for a histogram of the index lesion. Curr. Opin. Urol. 23, 118-22 (2013).
39. Baco, E. et al. Magnetic Resonance Imaging—Transectal Ultrasound Image-fusion Biopsies Accurately Characterize the Index Tumor: Correlation with Step-sectioned Radical Prostatectomy Specimens in 135 Patients. Eur. Urol. 67, 787-794 (2015).
40. Thompson, I. et al. Guideline for the management of clinically localized prostate cancer: 2007 update. J. Urol. 177, 2106-2131 (2007).
41. Washington, S. L., Bonham, M., Whitson, J. M., Cowan, J. E. & Carroll, P. R. Transrectal ultrasonography-guided biopsy does not reliably identify dominant cancer location in men with low-risk prostate cancer. BJU Int 110, 50-55 (2012).
42. Campan, M., Weisenberger, D. J., Trinh, B. & Laird, P. W. MethyLight. Methods Mol. Biol. 507, 325-337 (2009).
43. Bibikova, M. et al. High density DNA methylation array with single CpG site resolution. Genomics 98, 288-295 (2011).
44. Triche, T. J., Weisenberger, D. J., Van Den Berg, D., Laird, P. W. & Siegmund, K. D. Low-level processing of Illumina Infinium DNA Methylation BeadArrays. Nucleic Acids Res. 41, 1-11 (2013).
45. Lowe, R. & Rakyan, V. K. Marmal-aid—a database for Infinium HumanMethylation450. BMC Bioinformatics 14, 359 (2013).
46. Morris, T. J. et al. ChAMP: 450k Chip Analysis Methylation Pipeline. Bioinformatics 30, 428-30 (2014).
47. Desper, R. & Gascuel, O. Fast and Accurate Phylogeny Reconstruction Algorithms Based on the Minimum-Evolution Principle. J. Comput. Biol. 9, 687-705 (2002).
48. Quail D F, Joyce J A: Microenvironmental regulation of tumor progression and metastasis. Nat Med 2013, 19:1423-1437.
49. Damiano R, Di Lorenzo G, Cantiello F, De Sio M, Perdona S, D'Armiento M, Autorino R: Clinicopathologic features of prostate adenocarcinoma incidentally discovered at the time of radical cystectomy: an evidence-based analysis. Eur Urol 2007, 52:648-657.
50. Gakis G, Schilling D, Bedke J, Sievert K D, Stenzl A: Incidental prostate cancer at radical cystoprostatectomy: implications for apex-sparing surgery. BJU Int 2010, 105:468-471.
51. Cancer Genome Atlas Research Network. Electronic address scmo, Cancer Genome Atlas Research N: The Molecular Taxonomy of Primary Prostate Cancer. Cell 2015, 163:1011-1025.
52. Hernandez H G, Tse M Y, Pang S C, Arboleda H, Forero D A: Optimizing methodologies for PCR-based DNA methylation analysis. Biotechniques 2013, 55:181-197.
53. Haldrup C, Mundbjerg K, Vestergaard E M, Lamy P, Wild P, Schulz W A, Arsov C, Visakorpi T, Bone M, Hoyer S, et al: DNA methylation signatures for prediction of biochemical recurrence after radical prostatectomy of clinically localized prostate cancer. J Clin Oncol 2013, 31:3250-3258.
54. Kumar A, Coleman I, Morrissey C, Zhang X, True L D, Gulati R, Etzioni R, Bolouri H, Montgomery B, White T, et al: Substantial interindividual and limited intraindividual genomic diversity among tumors from men with metastatic prostate cancer. Nat Med 2016, 22:369-378.

1. A method of identifying subjects having prostate cancer comprising:
obtaining a sample from a subject;
isolating DNA from the sample;
determining the methylation status of the DNA; and
comparing the methylation status of the DNA to one or more methylated probes selected from the group consisting of cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013,
wherein the methylated probe comprises a sequence region that extends up to 250 base pairs upstream and downstream from the methylated probe, and
wherein the comparison indicates whether the sample is normal, a non-aggressive prostate cancer, or an aggressive prostate cancer.

2. The method of claim 1, wherein the sample is a biopsy sample.

3. The method of claim 2, wherein the biopsy is from a prostate.

4. The method of claim 2, wherein the biopsy is from a pelvic lymph node.

5. The method of claim 1, wherein two or more methylated probes are selected.

6. The method of claim 1, wherein the sample is selected from the group consisting of blood, plasma and urine.

7. The method of claim 1, wherein the sequence region extends up to 100 base pairs upstream and downstream from the methylated probe.

8. The method of claim 1, wherein the sequence region extends 0 base pairs upstream and downstream from the methylated probe.

9. The method of claim 1, wherein five or more methylated probes are selected.

10. The method of claim 1, wherein fifteen or more methylated probes are selected.

11. A method of classifying prostate cancer comprising:
obtaining a sample from a subject;
isolating DNA from the sample;
determining the methylation status of the DNA; and
comparing the methylation status of the DNA to one or more methylated probes selected from the group consisting of cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013,
wherein the methylated probe comprises a sequence region that extends up to 250 base pairs upstream and downstream from the methylated probe, and
wherein the comparison indicates whether the prostate cancer is non-aggressive or aggressive.

12. The method of claim 11, wherein the sample is a biopsy sample.

13. The method of claim 12, wherein the biopsy is from a prostate.

14. The method of claim 12, wherein the biopsy is from a pelvic lymph node.

15. The method of claim 11, wherein two or more methylated probes are selected.

16. The method of claim 11, wherein the sample is selected from the group consisting of blood, plasma and urine.

17. The method of claim 11, wherein the sequence region extends up to 100 base pairs upstream and downstream from the methylated probe.

18. The method of claim 11, wherein the sequence region extends 0 base pairs upstream and downstream from the methylated probe.

19-20. (canceled)

21. A method of identifying subjects having prostate cancer comprising:
obtaining a sample from a subject;
isolating DNA from the sample;
determining the methylation status of the DNA; and
comparing the methylation status of the DNA to one or more methylated probes selected from the group consisting of cg20399616, cg12737160, cg23624008, cg09789590, cg23095615, cg14273822, cg04294888, cg12098872, cg21981270, cg21526205, cg09806262, cg01940855, cg23855505, cg18423852, cg22260952, cg21883802, cg00036011, cg26573704, cg12727795 and cg01404317,
wherein the methylated probe comprises a sequence region that extends up to 250 base pairs upstream and downstream from the methylated probe, and
wherein the comparison indicates whether the sample is normal or prostate cancer.

22-40. (canceled)
